(19) United States
(12) Patent Application Publication
(10) Pub. No.: US 2024/0383898 A1
(43) Pub. Date: Nov. 21, 2024

(54) PYRIDO RING COMPOUND, PREPARATION METHOD THEREFOR, INTERMEDIATE, COMPOSITION, AND APPLICATION

(71) Applicant: YIYOU BIOTECH (SHANGHAI) CO., LTD, Shanghai (CN)

(72) Inventors: Zhiyu Li, Nanjing, Jiangsu (CN); Jinlei Bian, Nanjing, Jiangsu (CN); Tian Jing, Nanjing, Jiangsu (CN); Pengfei Xu, Nanjing, Jiangsu (CN); Pei Shen, Nanjing, Jiangsu (CN); Jubo Wang, Nanjing, Jiangsu (CN); Zhixia Qiu, Nanjing, Jiangsu (CN); Xi Xu, Nanjing, Jiangsu (CN)

(21) Appl. No.: 18/689,067

(22) PCT Filed: Dec. 21, 2021
(86) PCT No.: PCT/CN2021/140062

§ 371 Date: Mar. 4, 2024

(51) Int. Cl. C07D 471/14; A61K 31/437; A61K 31/444

(52) U.S. Cl. CPC C07D 471/14; A61K 31/437; A61K 31/444

(57) ABSTRACT

Disclosed are a pyrido ring compound, a preparation method therefor, an intermediate, a composition, and an application. The pyrido ring compound has a structure as shown in formula I, has JAK inhibitory activity, and can be used for treating JAK-related diseases, such as autoimmune diseases or cancers.

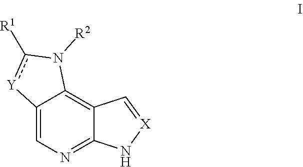

### TECHNICAL FIELD

The present invention relates to a pyridocyclic compound and its preparation method, intermediates, compositions and uses thereof.

### BACKGROUND

Janus kinases (JAK) belong to a family of tyrosine kinases involved in inflammation, autoimmune diseases, proliferative diseases, graft rejection, diseases associated with impaired cartilage renewal, congenital cartilage malformations and/or diseases associated with excessive IL6 secretion.

JAK kinases are cytoplasmic tyrosine kinases that transduce cytokine signals from membrane receptors to STAT transcription factors. The prior art has described four JAK kinase family members: JAK1, JAK2, JAK3, and TYK2. When cytokines bind to their receptors, JAK family members autophosphorylate and/or transphosphorylate each other, followed by phosphorylation of STATs, which then migrate into the nucleus to regulate transcription. JAK-STAT intracellular signaling applies to interferons, most interleukins, and a variety of cytokines and endocrine factors, such as EPO, TPO, GH, OSM, LIF, CNTF, GM-CSF, and PRL.

Combinatorial studies using genetic models and small molecule JAK inhibitors have revealed the therapeutic potential of several JAKs. JAK3 was confirmed as an immunosuppressive target by mouse and human genetics. JAK3 inhibitors were successfully used in clinical development, initially for organ transplant rejection but were subsequently used for other immunoinflammatory indications such as rheumatoid arthritis (RA), psoriasis, and Crohn's disease. TYK2 is a potential target for immunoinflammatory diseases and has been confirmed by human genetics and gene knockout studies in mice. JAK2 is currently an effective target in the treatment of myeloproliferative diseases, with two drugs for the treatment of myelofibrosis already on the market. JAK1 is a novel target in the field of immunoinflammatory diseases as the heterodimerization of JAK1 with other JAKs is known to transduce cytokine-driven pro-inflammatory signaling. Thus, the inhibition of JAK1 and/or other JAKs is expected to be therapeutically beneficial for a range of inflammatory conditions and other diseases driven by JAK-mediated signaling.

Tofacitinib was developed by Pfizer Inc. and successfully launched in the United States on Nov. 7, 2012 for the treatment of rheumatoid arthritis under the trade name Xeljanz.

Ruxolitinib, jointly developed by incyte and Novartis, was launched in the United States in 2011 for the treatment of myelofibrosis under the trade name Jakafi.

Baricitinib, jointly developed by Incyte and Eli Lilly, was launched in the United States in 2018 for the treatment of rheumatoid arthritis under the trade name Olumiant.

Upadacitinib, a selective JAK1 inhibitor developed by AbbVie and marketed in 2019 for the treatment of rheumatoid arthritis, has been reported in the literature to have activity of JAK1 IC₅₀=43 nM and JAK2 IC₅₀=200 nM.

Fedratinib is a selective JAK2 inhibitor developed by Sanofi and launched in 2019 for the treatment of myelofibrosis, has been reported in the literature to have an activity of JAK2 IC₅₀=3 nM.

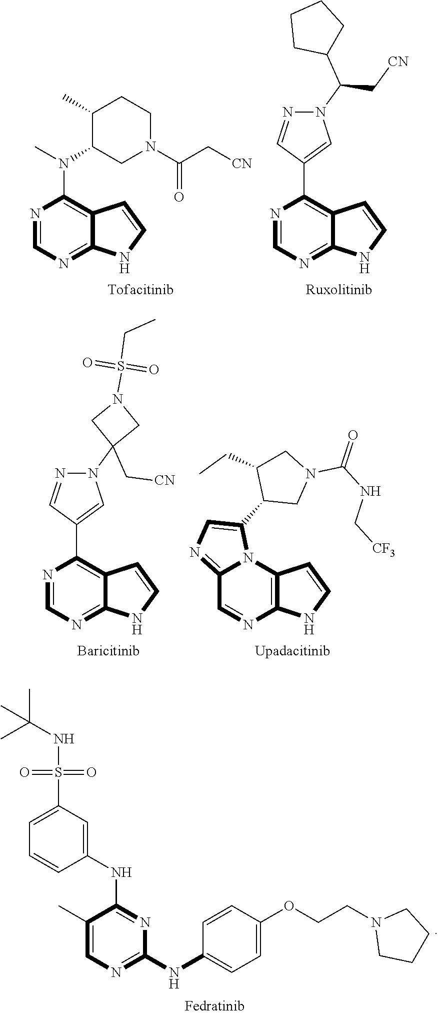

### CONTENT OF THE PRESENT INVENTION

The present invention provides a pyridocyclic compound and its preparation method, intermediates, compositions and uses.

On one hand, the present invention provides a compound shown in formula I.

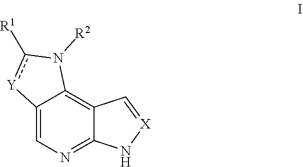

or a tautomer, stereoisomer, racemate or isotopic derivative thereof, or a pharmaceutically acceptable salt of any of the foregoing (referring to the foregoing compound as shown in Formula I, its tautomer, stereoisomer, racemate or isotopic derivative), or a crystalline or solvated form of any of the foregoing (referring to the foregoing compound as shown in Formula I, its tautomer, stereoisomer, racemate, isotopic derivative or pharmaceutically acceptable salt as shown in Formula I).
wherein X is CH or N.
Y is NH or N; when Y is N, the [symbol] connected to Y is a double bond; when Y is NH, the [symbol] connected to Y is a single bond.
R¹ and R² are defined as (i), (ii) or (iii) as follows.
(i) R¹ is

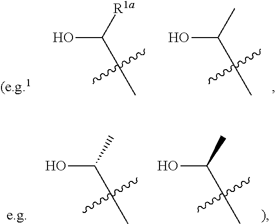

R² is

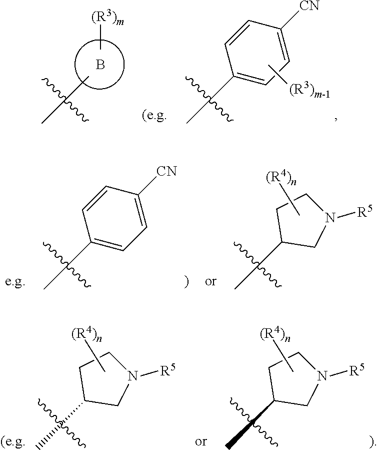

Ring B is a benzene ring or a 5-6-membered heteroaromatic ring.
R^{1a} is a C₁₋₃ alkyl group (e.g., methyl group).

Each R³ is independently a halogen (e.g., fluorine), a cyano group, a C₁₋₄ alkyl group (e.g., methyl group), a CIA haloalkyl group (e.g., C₁₋₄ fluoroalkyl group, e.g., —CF₃), a —O—C₁₋₄ alkyl group (e.g., —O—CH₃), or a —O—C₁₋₄ haloalkyl group (e.g. a —O—C₁₋₄ fluoroalkyl group, e.g. —OCF₃).

Each R⁴ is independently a halogen (e.g., fluorine), a hydroxyl group, a C₁₋₄ alkyl group (e.g., methyl group), a C₁₋₄ haloalkyl group (e.g., C₁₋₄ fluoroalkyl group, e.g., —CF₃), an —O—C₁₋₄ alkyl group (e.g., —O—CH₃), a —O—C₁₋₄ haloalkyl group (e.g. —O—C₁₋₄ fluoroalkyl group, e.g. —OCF₃) or a C₁₋₄ hydroxyalkyl group (e.g. —CH₂OH).

Alternatively, two R⁴ located on the same carbon atom or on different carbon atoms are interconnected to form —CH₂— or —(CH₂)₂— (e.g., to form

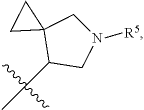

for example

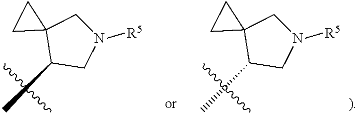

R⁵ is —S(O)₂R^{5a}, —C(O)R^{5b}, —C(O)NR^{5c}R^{5d}, —C(O)OR^{5c}. —C(O)NHR^{5k} or -L¹-R^{5f};
R^{5a} is a C₂₋₆ alkyl group (e.g. ethyl, n-propyl or n-butyl group), a C₂₋₆ haloalkyl group, -L¹-R^{5f}, an unsubstituted or substituted 6-10-membered aryl group (said 6-10-membered aryl group e.g. phenyl or naphthyl group) or an unsubstituted or substituted 5-10-membered heteroaryl group (said 5-10-membered heteroaryl group e.g. 5-6-membered heteroaryl group), said substituted 6-10-membered aryl group and substituted 5-10-membered heteroaryl group are defined as structures where 1, 2, 3 or 4 hydrogen atoms in the 6-10-membered aryl group and the 5-10-membered heteroaryl group are independently substituted by R^{5g}.

R^{5b}, R^{5c}, R^{5d}, R^{5e} and R^{5k} are each independently a C₁₋₆ alkyl group, a C₁₋₆ haloalkyl group, -L¹-R^{5f}, an unsubstituted or substituted 6-10-membered aryl group (said 6-10-membered aryl group e.g. phenyl or naphthyl group) or an unsubstituted or substituted 5-10 membered heteroaryl group (said 5-10-membered heteroaryl group e.g. 5-6-membered heteroaryl group), said substituted 6-10-membered aryl group and substituted 5-10-membered heteroaryl group are defined as structures where 1, 2, 3 or 4 hydrogen atoms in the 6-10-membered-aryl group and the 5-10-membered-heteroaryl group are independently substituted by R^{5h}.

Each R^{5g} and R^{5h} is independently a halogen (e.g., fluorine), a cyano group, a C₁₋₄ alkyl group (e.g., methyl group), a C₁₋₄ haloalkyl group (e.g., a C₁₋₄ fluoroalkyl group, e.g., —CF₃), a —O—C₁₋₄ alkvl group (e.g., —O—CH₃), a —O—C₁₋₄ haloalkyl group (e.g. —OCF₃) or a phenyl group.

Each L¹ is independently —[C(R^{a}R^{b})]₁₋₅— (e.g. —CH₂—, —(CH₂)₂—, —(CH₂)₃—, —(CH₂)₄—, —(CH₂)₅— or

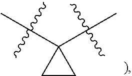 [SMILES: *C1(*)CC1]

—[C(R^{a}R^{b})]₁₋₂—C(O)—[C(R^{a}R^{b})]₁₋₂—, —[C(R^{a}R^{b})]₁₋₂—C(O)NH—[C(R^{a}R^{b})]₁₋₂— (e.g. —C(R^{a}R^{b})—C(O)NH—C(R^{a}R^{b})—R^{5f}), —[C(R^{a}R^{b})]₁₋₂—NHC(O)—[C(R^{a}R^{b})]₁₋₂—, —[C(R^{a}R^{b})]₁₋₂—S(O)₂—[C(R^{a}R^{b})]₁₋₂—, —[C(R^{a}R^{b}))]₁₋₂—NHS(O)₂—[C(R^{a}R^{b})]₁₋₂— or —[C(R^{a}R^{b})]₁₋₂—S(O)₂NH—[C(R^{a}R^{b})]₁₋₂—.

Each R^{5f} is independently H, F, CHF₂, CH₂F, CF₃ or CN.

Each R^{a} is independently H, a halogen (e.g., fluorine), or a C₁₋₃ alkyl group (e.g., methyl or ethyl group).

Each R^{b} is independently H, a halogen (e.g., fluorine), or a C₁₋₃ alkyl group (e.g., methyl or ethyl group).

Alternatively, R^{a} and R^{b} along with the carbon atom joining them, form a cyclopropyl group.

m is 0, 1, 2, 3 or 4.

n is 0, 1, 2, 3 or 4.

(ii) R¹ is

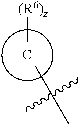

### and R² is

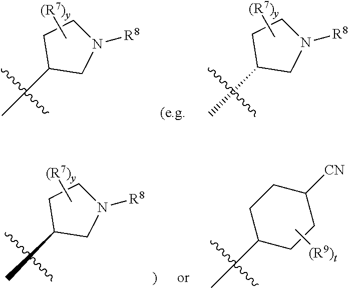

(e.g. trans or cis).

Ring C is a benzene ring or a 5-6-membered heteroaromatic ring (e.g., imidazole, thiazole, furan, thiophene, or pyridine, e.g.,

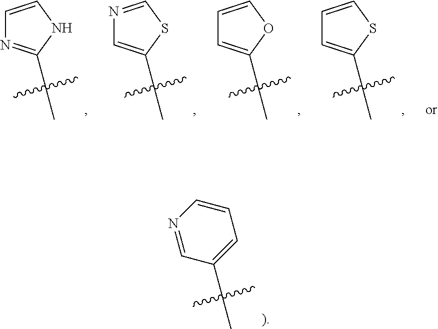 [SMILES: *c1ncc[nH]1 ; *c1cncs1 ; *c1ccco1 ; *c1cccs1 ; *c1cccnc1]

Each R⁶ is independently a halogen (e.g., fluorine or chlorine, e.g., fluorine), a hydroxyl group, an amino group, a cyano group, a C₁₋₄ alkyl group (e.g., methyl group), a C₁₋₄ haloalkyl group (e.g., C₁₋₄ fluoroalkyl group, e.g., CF₃), an —O—C₁₋₄ alkyl group (e.g., —O—CH₃), an O—C₁₋₄ haloalkyl group (e.g. —O—C₁₋₄ fluoroalkyl group, e.g. —O—CF₃), a —S—C₁₋₄ alkyl group (e.g. —S—CH₃), a —S(O)₂—C₁₋₄ alkyl group (e.g. —S(O)₂—CH₃) or a C₁₋₄ hydroxyalkyl group (e.g. —CH₂—OH).

Each R⁷ is independently R⁴ (i.e., R⁷ and R⁴ in the compound as shown in Formula I in any of the embodiments of the present invention have the same definition).

Alternatively, two R⁷ located on the same carbon atom or on different carbon atoms are interconnected to form —CH₂— or —(CH₂)₂—.

R⁸ is R⁵ (i.e., R⁸ and R⁵ in the compound as shown in Formula I in any of the embodiments of the present invention have the same definition), —S(O)₂R^{8a}, —C(O)R^{8b}, —C(O)NR^{8c}R^{8d}, —C(O)OR^{8e} or —C(O)NHR^{8k}.

R^{8a}, R^{8b}, R^{8c}, R^{8d}, R^{8e} and R^{8k} are each independently a methyl group, —CF₃, a C₂₋₆ alkenyl group (e.g. vinyl group) or a C₃₋₆, cycloalkyl group (e.g. cyclopropyl, cyclobutyl or cyclopentyl group).

Each R⁹ is independently R⁴.

Alternatively, two R⁹ located on the same carbon atom or on different carbon atoms are interconnected to form —CH₂— or —(CH₂)₂—.

z is 0, 1, 2, 3 or 4.

y is 0, 1, 2, 3 or 4.

t is 0, 1, 2, 3 or 4.

(iii) R¹ is H, CF₃ or

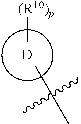

### and R² is

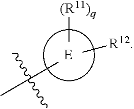

Ring D is a C₃₋₆ cycloalkyl group (e.g., cyclopropyl or cyclobutyl group), a benzene or a 5-6-membered heteroaromatic ring (e.g., furan or thiophene, e.g.,

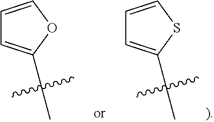 [SMILES: *c1ccco1 ; *c1cccs1]

Ring E is a benzene ring or a 5-6-membered heteroaromatic ring (e.g., furan or thiophene, e.g.,

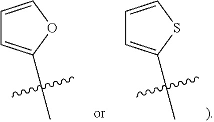 [SMILES: *c1ccco1 ; *c1cccs1]

Each R¹⁰ and R¹¹ is independently a halogen (e.g., fluorine or chlorine, e.g., fluorine), a hydroxyl group, an amino group, a cyano group, a C₁₋₄ alkyl group (e.g., methyl group), a C₁₋₄ haloalkyl group (e.g., C₁₋₄ fluoroalkyl group, e.g., CF₃), a —O—C₁₋₄ alkyl group (e.g., —O—CH₃), a —O—C₁₋₄ haloalkyl group (e.g. —O—C₁₋₄ fluoroalkyl group, e.g. —O—CF₃), a —S—C₁₋₄ alkyl group (e.g. —S—CH₃), a —S(O)₂—C₁₋₄ alkyl group (e.g. —S(O)₂—CH₃) or a C₁₋₄ hydroxyalkyl group (e.g. —CH₂—OH).

R¹² is a cyano group, -L³-R^{12f} or

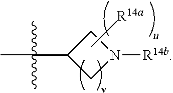

Each R^{14a} is independently a halogen (e.g., fluorine), a cyano group, a C₁₋₄ alkyl group (e.g., methyl group), a cyano-substituted C₁₋₄ alkyl group (e.g., —CH₂CN), a C₁₋₄ haloalkyl group (e.g., C₁₋₄ fluoroalkyl group, e.g., CF₃), a —O—C₁₋₄ alkyl group (e.g., —O—CH₃), a —O—C₁₋₄ haloalkyl group (e.g. —O—C₁₋₄ fluoroalkyl group, e.g. —O—CF₃), or a C₁₋₄ hydroxyalkyl group (e.g. —CH₂OH).

R^{14b} is R⁵ (i.e., R^{14b} and R⁵ in the compound in any of the embodiments of the present invention as shown in Formula I have the same definition) or —S(O)₂—C₁₋₄ alkyl (e.g., —S(O)₂—CH₂CH₃, —S(O)₂—CH₂CH₂CH₃ or —S(O)₂—CH₂CH₂CH₂CH₃).

L³ is —[C(R^{e}R^{f})]₁₋₅— (e.g. —CH₂—, —(CH₂)₂—, —(CH₂)₃—, —(CH₂)₄— or —(CH₂)₅—), —C(O)—, —C(O)NH—, —NHC(O)—, —S(O)₂—, —NHS(O)₂—, —S(O)₂NH—, —[C(R^{e}R^{f})]₁₋₂—C(O)—[C(R^{e}R^{f})]₁₋₂, —[C(R^{e}R^{f})]₁₋₂—C(O)NH—[C(R^{e}R^{f})]₁₋₂—, —[C(R^{e}R^{f})]₁₋₂—NHC(O)—[C(R^{e}R^{f})]₁₋₂—, —[C(R^{e}R^{f})]_{1- 2}—S(O)₂[C(R^{e}R^{f})]₁₋₂—, —[C(R^{e}R^{f})]₁₋₂—NHS(O)₂—[C(R^{e}R^{f})]₁₋₂— or —[C(R^{e}R^{f})]₁₋₂—S(O)₂NH—[C(R^{e}R^{f})]₁₋₂—.

R^{12f} is H, F, CHF₂, CH₂F, CF₃ or CN.

Each R^{e} and R^{f} is independently H, a halogen (e.g., fluorine), a C₁₋₃ alkyl group (e.g., methyl or ethyl group), or a C₃₋₆ cycloalkyl group (e.g., cyclopropyl, cyclobutyl, or cyclopentyl group).

Alternatively, R^{e} and R^{f} along with their connecting carbon atom, form a cyclopropyl group.

p is 0, 1, 2, 3 or 4.

q is 0, 1, 2, 3 or 4.

v is 0, 1, 2 or 3.

u is 0, 1 or 2.

The heteroatom in the above heteroaryl group is independently N, O or S, and the number of heteroatoms in the above heteroaryl group is independently 1, 2, 3 or 4.

In some embodiments, said compound shown in formula I has the structure shown in formula I-1 as follows.

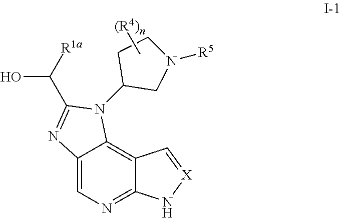

wherein R^{1a}, R⁴, R₅, X and n are defined as described in the compound shown in Formula I as defined in any of the embodiments of the present invention.

In some embodiments, a compound as shown in Formula I or I-1 has a structure as shown in Formula I-1C, I-1D, I-1E or I-1F as follows.

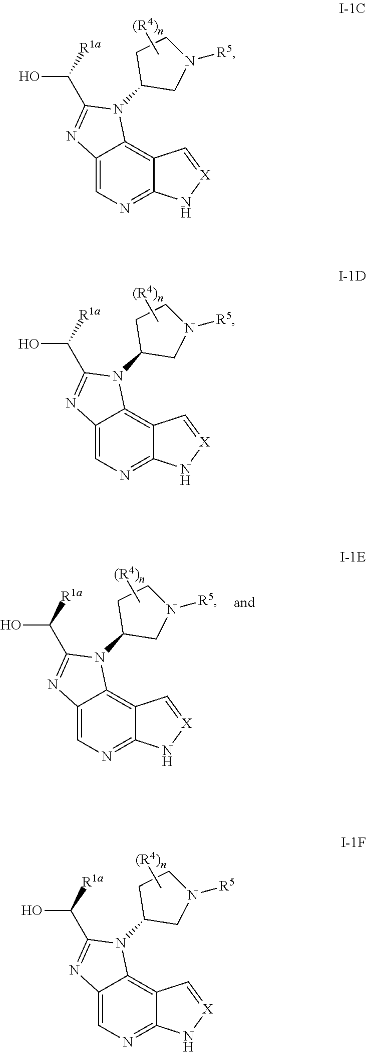

wherein R^{1a}, R⁴, R⁵, X and n are defined as described in the compound shown in Formula I as defined in any of the embodiments of the present invention.

In some embodiments, said compound shown in formula I or I-1 has the structure shown in formula I-1a, I1b, I-1c or I-1d as follows, preferably having the structure shown in I-1d.

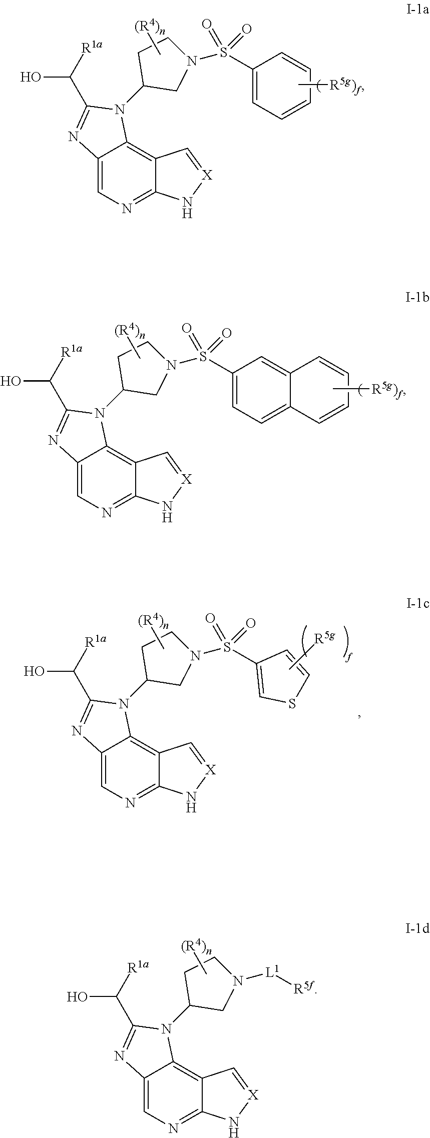

wherein R^{1a}, R⁴, R^{5g}, X, n, L¹ and R^{5f} are defined as described in the compound shown in Formula I as defined in any of the embodiments of the present invention.

Each f is independently 0, 1, 2, 3 or 4, e.g. 0 or 1.

In some embodiments, said compound shown in formula I has the structure shown in formula I-2, I-2A or I-2B as follows

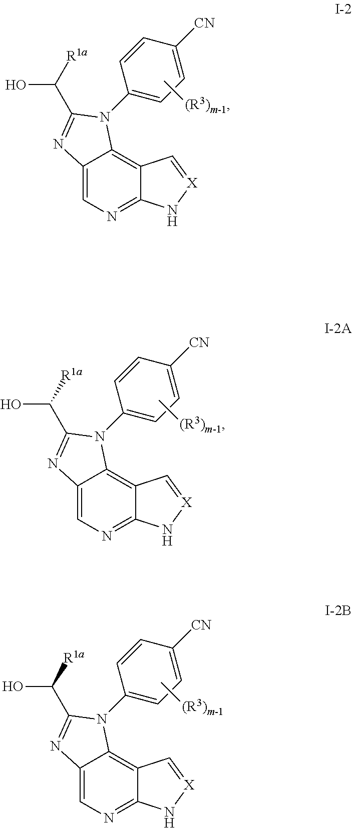

wherein R^{1a}, R³, X and m are defined as described in the compound shown in Formula I as defined in any of the embodiments of the present invention.

In some embodiments, as in the compounds shown in Formula I, I-1, I-1C, I-1D, I-1E, I-1F, 1-1a, I-1b, 1-1c, 1-1d, I-2, I-2A, or I-2B, X is CH.

In some embodiments, as in the compounds shown in Formula I, I-1, 1-1C, 1-1D, I-1E, I-1F, 1-1a, I-1b, I-1c, I-1d, I-2, I-2A, or I-2B, X is N.

In some embodiments, as in the compounds shown in Formula I, I-1, I-1C, I-1D, I-1E, I-1F, I-1a, I-1b, I-1c, I-1d, I-2, I-2A, or I-2B, R^{1a} is a methyl group.

In some embodiments, as in the compounds shown in Formula I, I-1, I-1a, I-1b, I-1c, I-1d, or I-2,

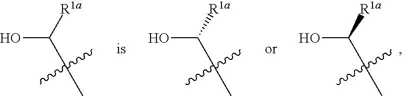

preferably

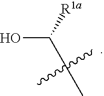

(i) In some embodiments, as in the compounds shown in Formula I, I-2, I-2A or I-2B, m is 1.

In some embodiments, as in the compounds shown in Formula I, I-1, I-1a, I-1b, I-1c, or I-1d,

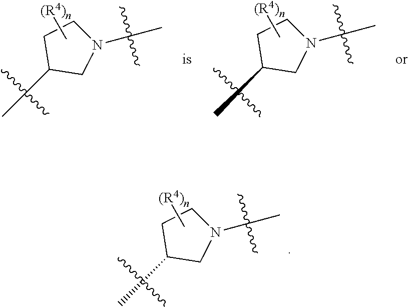

In some embodiments, is

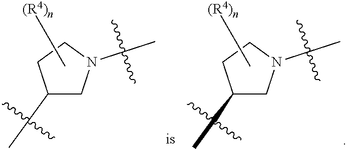

In some embodiments,

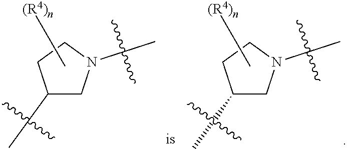

In some embodiments, as in the compounds shown in Formula I, I-1, I-1C, I-1D, I-1E, I-1F, I-1a, I-1b, I-1c or I-1d, each R⁴ is independently a halogen (e.g., fluorine) or a C₁₋₄ hydroxyalkyl group (e.g., —CH₂OH), or, alternatively, two R⁴ located at the same carbon atom or at different carbon atoms are interconnected to form —CH₂— or —(CH₂)₂—.

In some embodiments, as in the compounds shown in Formula I, I-1, I-1C, I-1D, I-1E, I-1F, I-1a, I-1b, I-1c, I-1d, I-2, I-2A, or I-2B, n is 0, 1, or 2.

In some embodiments, as in the compounds shown in Formula I, I-1, I-1C, I-1D, I-1E, I-1F, I-1a, I-1b, I-1c, or I-1d,

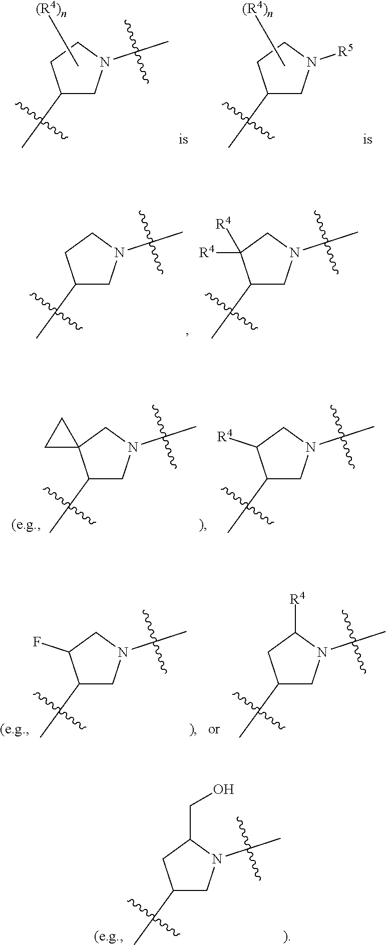

In some embodiments, as in the compounds shown in Formula I, I-1, I-1C, I-1D, I-1E, I-1F, I-1a, I-1b, I-1c, or I-1d,

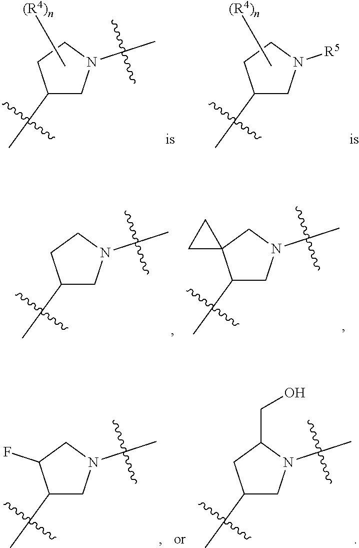

In some embodiments, the ring B is a benzene ring in the compound as shown in Formula 1.

In some embodiments, m is 1 in the compound as shown in Formula I.

In some embodiments, as in the compounds shown in Formula I, I-1, I-1C, I-1D, I-1E or I-1F, R^{5a} is a C₂₋₆ alkyl group (e.g., ethyl, n-propyl or n-butyl group), an unsubstituted or substituted 6-10-membered aryl group (said 6-10-membered aryl group e.g., phenyl or naphthyl group) or an unsubstituted or substituted 5-10-membered heteroaryl group (said 5-10-membered heteroaryl group e.g. 5-6-membered heteroaryl group).

In some embodiments, as in the compounds shown in Formula I, I-1, I-1C, I-1D, I-1E, or I-1F, the “unsubstituted or substituted 6-10-membered aryl group” in the definition of R^{5a} may be “unsubstituted or substituted phenyl group”, or “unsubstituted or substituted naphthyl group”, wherein said unsubstituted or substituted phenyl group may be, for example, an unsubstituted phenyl group, or a phenyl group substituted with 1, 2, 3 and 4 (e.g., 1) of the functional groups independently selected from the following: halogens (e.g., fluorine), C₁₋₄ alkyl groups (e.g., methyl), —O—C₁₋₄ haloalkyl groups (e.g., —OCF₃) and phenyl groups, such as

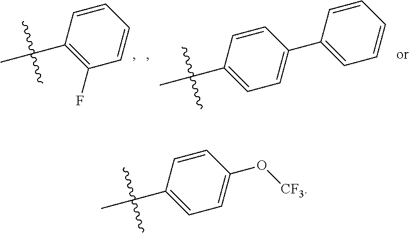

Wherein, said unsubstituted or substituted naphthyl group may be, for example, an unsubstituted naphthyl group, such as

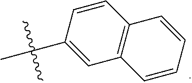 [SMILES: *c1ccc2ccccc2c1]

In some embodiments, the “unsubstituted or substituted 6-10-membered-aryl group” in R^{5a} is preferably a —OCF₃ substituted phenyl group

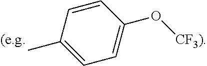

In some embodiments, as in the compounds shown in Formula I, I-1, I-1C, I-1D, I-1E or I-1F, “unsubstituted or substituted 5-10-membered-heteroaryl group” in the definition of R^{5a} may be “unsubstituted or substituted 5-6-membered heteroaryl group”, such as an unsubstituted or substituted thiophenyl group, preferably

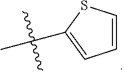 [SMILES: *c1cccs1]

In some embodiments, as in the compound shown in Formula I, I-1, I-1C, I-1D, I-1E, I-1F, I-1a, I-1b, or I-1c, each R^{5g} is independently a halogen (e.g., fluorine), a C₁₋₄ alkyl group (e.g., methyl group), a —O—C₁₋₄ haloalkyl group (e.g. —OCF₃) or a phenyl group, preferably —OCF₃.

In some embodiments, as in the compounds shown in Formula I, I-1, I-1C, I-1D, I-1E or I-1F, —S(O)₂R^{5f} in the definition of R⁵ is —S(O)₂(CH₂)₂—CF₃, —S(O)₂(CH₂)₂—CH₃, —S(O)₂CH₂—CH₃, —S(O)₂(CH₂)₃—CH₃, —S(O)₂(CH₂)₂—CF₃,

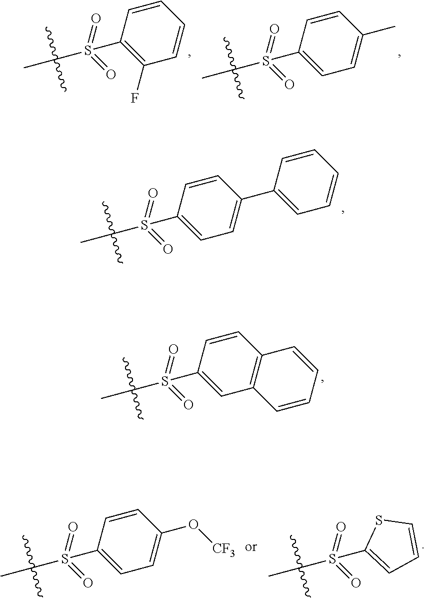

preferably —S(O)₂(CH₂)₂—CF₃, —S(O)₂(CH₂)₂—CH₃, —S(O)₂CH₂—CH₃, —S(O)₂(CH₂)₃—CH₃, —S(O)₂(CH₂)₂—CF₃, or

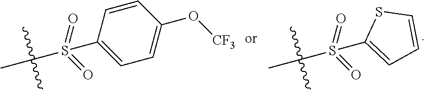 [SMILES: *S(=O)(=O)c1ccc(OC(F)(F)F)cc1 ; "or" ; *S(=O)(=O)c1cccs1 ; "."]

In some embodiments, as in the compounds shown in Formula I, I-1, I-1C, I-1D, I-1E, or I-1F, R^{5b} is -L¹-R^{5f}, or an unsubstituted or substituted phenyl group.

In some embodiments, as in the compounds shown in Formula I, I-1, I-1C, I-1D, I-1E, or I-1F, -L¹-R^{5f}, in the definition of R^{5b} is —C(R^{a}R^{b})—R^{5f}, for example

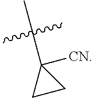 [SMILES: *C1(C#N)CC1]

In some embodiments, as in the compounds shown in Formula I, I-1, I-1C, I-1D, I-1E or I-1F, the “unsubstituted or substituted 6-10-membered aryl group” in the definition of R^{5b} may be “unsubstituted or substituted phenyl group”, or “unsubstituted or substituted naphthyl group”; wherein unsubstituted or substituted phenyl group may be, for example, an unsubstituted phenyl group, or a phenyl group substituted with 1, 2, 3 and 4 (e.g., 1) of the functional groups independently selected from the following: halogens (e.g., fluorine) and cyano-substituted phenyl groups such as

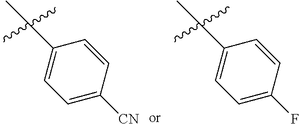 [SMILES: *c1ccc(C#N)cc1 ; "or" ; *c1ccc(F)cc1]

In some embodiments, as in the compounds shown in Formula I, I-1, I-1C, I-1D, I-1E, or I-1F, the —C(O)R^{5b} in the definition of R⁵ is

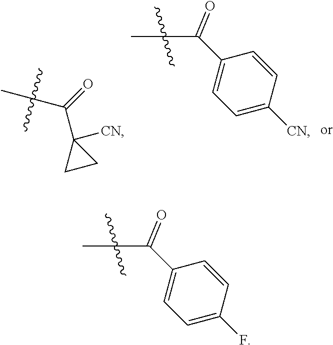 [SMILES: *C(=O)C1(C#N)CC1 ; *C(=O)c1ccc(C#N)cc1 ; "or" ; *C(=O)c1ccc(F)cc1]

In some embodiments, as in the compounds shown in Formula I, I-1, I-1C, I-1D, I-1E, or I-1F, R^{5e} is a C₁₋₆ alkyl group, such as tert-butyl group.

In some embodiments, as in the compounds shown in Formula I, I-1, I-1C, I-1D, I-1E, or I-1F, —C(O)OR^{5e} in the definition of R⁵ is —C(O)OC(CH₃)₂—CH₃.

In some embodiments, as in the compounds shown in Formula I, I-1, I-1C, I-1D, I-1E, or I-1F, R^{5c}, R^{5d}, and R^{5k} are each independently -L¹-R^{5f}.

In some embodiments, as in the compounds shown in Formula I, I-1, I-1C, I-1D, I-1E or I-1F, -L¹-R^{5f} in the definitions of R^{5c}, R^{5d} and R^{5k} are —C(R^{a}R^{b})—R^{5f}, e.g. —CH₂—R^{5f}, e.g. —CH₂—CF₃.

In some embodiments, as in the compounds shown in Formula I, I-1, I-1C, I-1D, I-1E, or I-1F, —C(O)NHR^{5k} in the definition of R⁵ is —C(O)NHC(R^{a}R^{b})—R^{5f}, e.g. —C(O)NHCH₂-R^{5f}, e.g. —C(O)NHCH₂—CF₃.

In some embodiments, as in the compounds shown in Formula I, I-1, I-1C, I-1D, I-1E or I-1F, -L¹-R^{5f} in the definition of R⁵ is —[C(R^{a}R^{b})]₁₋₅—R^{5f} or —[C(R^{a}R^{b})]₁₋₂—C(O)NH—[C(R^{a}R^{b})]₁₋₂—, preferably —C(R^{a}R^{b})—R_{5f}, —[C(R^{a}R^{b})]₂—R^{5f}, —[C(R^{a}R^{b})]₃—R^{5f}, —[C(R^{a}R^{b})]₄-R^{5f} or —[C(R^{a}R^{b})]₅-R^{5f}, further preferably —[C(R^{a}R^{b})]₂—R^{5f}, —[C(R^{a}R^{b})]₃-R^{5f}.

In some embodiments, as in the compounds shown in Formula I, I-1, I-1C, I-1D, I-1E, or I-1F, -L¹-R^{5f} in the definition of R⁵ is —C(R^{a}R^{b})—R^{5f}, —[C(R^{a}R^{b})]₂—R^{5f}, —[C(R^{a}R^{b})]₃—R^{5f}, —[C(R^{a}R^{b})]₄—R^{5f}, —[C(R^{a}R^{b})]₅-R^{5f} or —C(R^{a}R^{b})—C(O)NH—C(R^{a}R^{b})—R^{5f}, preferably —[C(R^{a}R^{b})]₂—R^{5f} or —[C(R^{a}R^{b})₃-R^{5f}.

In some embodiments, as in the compounds shown in Formula I, I-1, I-1C, I-1D, I-1E, or I-1F, each R^{5f} is independently F, CF₃, or CN. In some embodiments, each R^{5f} is independently F. In some embodiments, each R^{5f} is independently CF₃. In some embodiments, each R^{5f} is independently CN.

In some embodiments, as in the compounds shown in Formula I, I-1, I-1C, I-1D, I-1E, or I-1F, -L¹-R^{5f} in the definition of R⁵ is —C(R^{a}R^{b})—CN, —[C(R^{a}R^{b})]₂—CN, —[C(R^{a}R^{b})]₃—CN, —[C(R^{a}R^{b})]₄—CN, —[C(R^{a}R^{b})]—CF₃, —[C(R^{a}R^{b})]₂—CF₃, —[C(R^{a}R^{b})]₃—CF₃, —C(R^{a}R^{b})C(O)NH—C(R^{a}R^{b})CF₃ or —[C(R^{a}R^{b})]₅—F, preferably —C(R^{a}R^{b})—CN, —[C(R^{a}R^{b})]₂—CN, —[C(R^{a}R^{b})]₃—CN, —[C(R^{a}R^{b})]₄—CN, —[C(R^{a}R^{b})]—CF₃, —[C(R^{a}R^{b})]₂—CF₃, —[C(R^{a}R^{b})]₃—CF₃, or —[C(R^{a}R^{b})]₅—F.

In some embodiments, as in the compounds shown in Formula I, I-1, I-1C, I-1D, I-1E or I-1F, each R^{a} is independently H, fluorine, a methyl or ethyl group; each R^{b} is independently H, fluorine, a methyl or ethyl group; or, R^{a} and R^{b} along with the carbon atom connecting them, form a cyclopropyl group together.

In some embodiments, as in the compounds shown in Formula I, I-1, I-1C, I-1D, I-1E or I-1F, each R^{b} is independently H, fluorine, a methyl group or an ethyl group.

In some embodiments, as in the compounds shown in Formula I, I-1, I-1C, I-1D, I-1E or I-1F, each R^{b} is independently H or fluorine.

In some embodiments, as in the compounds shown in Formula I, I-1, I-1C, I-1D, I-1E or I-1F, each R^{b} is independently H, fluorine, a methyl group or an ethyl group.

In some embodiments, as in the compounds such as those shown in Formula I, I-1, I-1C, I-1D, I-1E or I-1F, each R^{b} is independently H, fluorine, a methyl group or an ethyl group.

In some embodiments, as in the compounds shown in Formula I, I-1, I-1C, I-1D, I-1E or I-1F, -L¹-R^{5f} in the definition of R⁵ is —CH₂—CN, —(CH₂)₂—CN, —(CH₂)₃—CN, —(CH₂)₄—CN, —(CH₂)₂—CF₃, —CH₂(CF₂)₂—CF₃, —(CF₂)₃—CF₃, —(CH₂)₂CF₂—CF₃, —(CH₂)₃—CF₃, —CH(CH₂CH₃)C(O)NH—CH₂CF₃, —CH₂C(O)NHCH₂—CF₃, —CH(CH₃)C(O)NHCH₂—CF₃ or —(CH₂)₅—F, preferably —(CH₂)₂—CN, —(CH₂)₃—CN, —(CH₂)₂—CF₃, —CH₂(CF₂)₂—CF₃, —(CF₂)₃—CF₃, —(CH₂)₂CF₂—CF₃ or —(CH₂)₃—CF₃.

In some embodiments, as in the compounds shown in Formula I, I-1, I-1C, I-1D, I-1E, or I-1F, R⁵ is —CH₂—CN, —(CH₂)₂—CN, —(CH₂)₃—CN, —(CH₂)₄—CN, —(CH₂)₂—CF₃, —CH₂(CF₂)₂—CF₃, —(CF₂)₃—CF₃, —(CH₂)CF₂—CF₃, —(CH₂)₃—CF₃, —CH(CH₂CH₃)C(O)NH—CH₂CF₃, —CH₂C(O)NHCH₂—CF₃, —CH(CH₃)C(O)NHCH₂—CF₃, —(CH₂)₅—F,

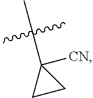 [SMILES: *C1(C#N)CC1]

—S(O)₂(CH₂)₂—CF₃, —C(O)NHCH₂—CF₃, —S(O)₂(CH₂)₂—CH₃, —S(O)₂CH₂—CH₃, —S(O)₂(CH₂)₃—CH₃, —S(O)₂(CH₂)₂—CF₃,

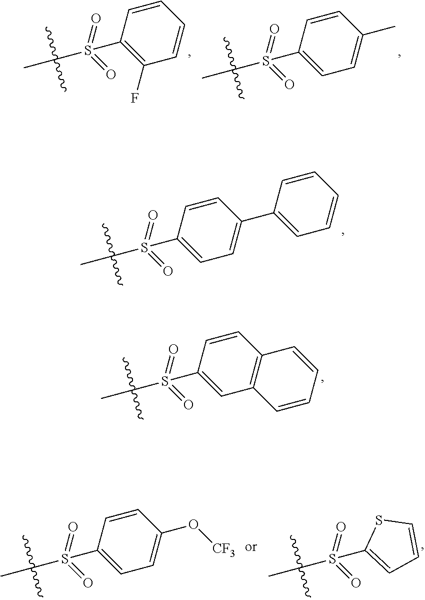

C(O)OC(CH₃)₂—CH₃, or

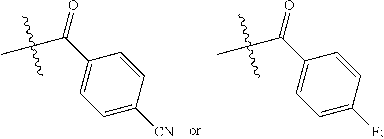 [SMILES: *C(=O)c1ccc(C#N)cc1 ; "or" ; *C(=O)c1ccc(F)cc1]

preferably —CH₂—CN, —(CH₂)₂—CN, —(CH₂)₃—CN, —(CH₂)₄—CN, —(CH₂)₂—CF₃, —CH₂(CF₂)₂—CF₃, —(CF₂)₃—CF₃, —(CH₂)₂CF₂—CF₃, —(CH₂)₃—CF₃, —CH(CH₂CH₃)C(O)NH—CH₂CF₃, —CH₂C(O)NHCH₂—CF₃, —CH(CH₃)C(O)NHCH₂—CF₃, —(CH₂)₅—F, —S(O)₂(CH₂)₂—CF₃, —S(O)₂CH₂—CH₃, —S(O)₂(CH₂)₃—CH₃, —S(O)₂(CH₂)₂—CF₃,

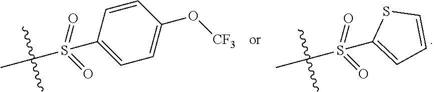 [SMILES: *S(=O)(=O)c1ccc(OC(F)(F)F)cc1 ; *S(=O)(=O)c1cccs1]

In some embodiments, said compound shown in formula I has the structure shown in formula I-3, I-3A or I-3B as follows.

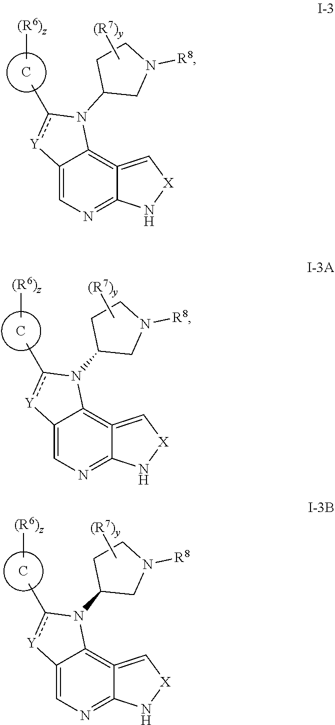

wherein X, Y, [symbol] , ring C, R⁶, R⁷, R⁹, z and y are defined as described in the [symbol] compound shown in Formula I as defined in any of the embodiments of the present invention.

In some embodiments, said compound shown in formula I has the structure shown in formula I-4 as follows.

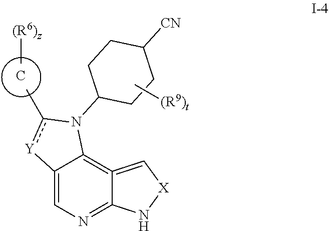

wherein X, Y, [symbol] , ring C, R⁶, R⁹, z and t are defined as described in the compound shown in Formula I as defined in any of the embodiments of the present invention.

In some embodiments, as in the compounds shown in Formula I, I-3, I-3A, I-3B, or I-4, [symbol] is a double bond and Y is N.

In some embodiments, as in the compounds shown in Formula I, I-3, I-3A, I-3B, or I-4, [symbol] is a single bond and Y is NH.

In some embodiments, as in the compounds shown in Formula I, I-3, I-3A, I-3B, or I-4, the 5-6-member e r aromatic ring in the definition of ring C is an imidazole

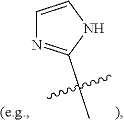 [SMILES: *c1ncc[nH]1]

thiazol

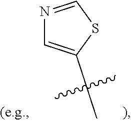 [SMILES: *c1cncs1]

furan

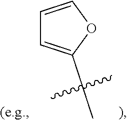 [SMILES: *c1ccco1]

thiophene

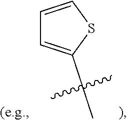 [SMILES: *c1cccs1]

or pyridine

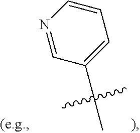 [SMILES: *c1cccnc1]

In some embodiments, as in the compounds shown in Formula I, I-3, I-3A, I-3B, I-4, I-4A, I-4, I-4C or I-4D, when R² is

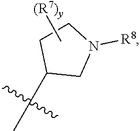

ring C is a benzene ring, an imidazole

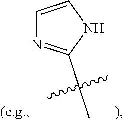 [SMILES: *c1ncc[nH]1]

a furan

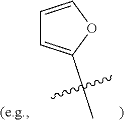 [SMILES: *c1ccco1]

or a pyridine

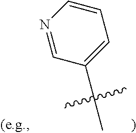 [SMILES: *c1cccnc1]

In some embodiments, as in the compounds shown in Formula I, I-3, I-3A, I-3B, I-4, I-4A, I-4B, I-4C or I4D, when R² is

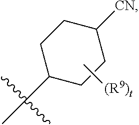

ring C is a benzene ring, a thiazole

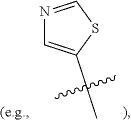 [SMILES: *c1cncs1]

a furan

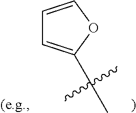 [SMILES: *c1ccco1]

or a thiophene

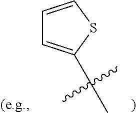 [SMILES: *c1cccs1]

In some embodiments, as in the compounds shown in Formula I, I-3, I-3A, I-3B, I-4 I-4A, I-4B, I-4C, or I-4D

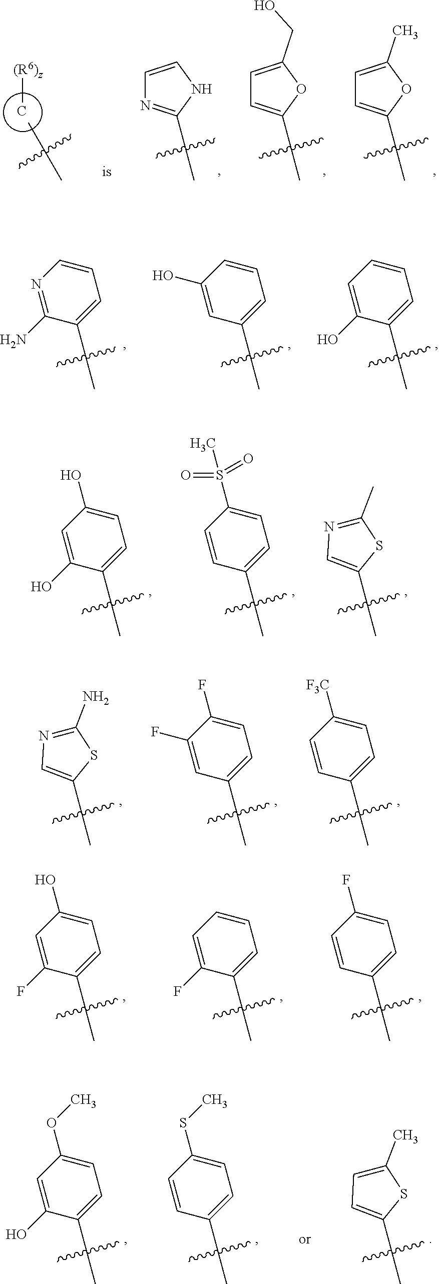

In some embodiments, said compound shown in formula I has the structure shown in formula I-3C, I-3D, I-3E, I-3F or I-3G as follows.

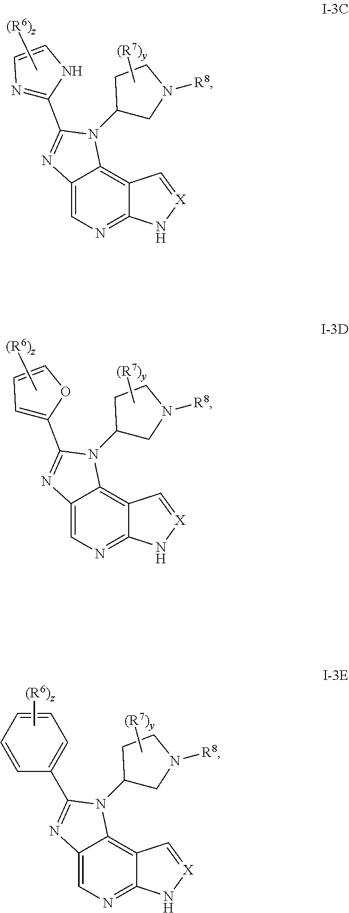

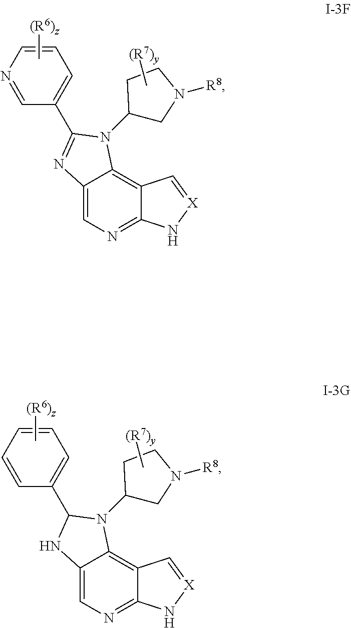

wherein X, R⁶, R⁷, R⁸, z and y are defined as described in any of the embodiments of the present invention.

In some embodiments, as in the compounds shown in Formula I, I-3, I-3C, I-3D, I-3E, I-3F, or I-3G,

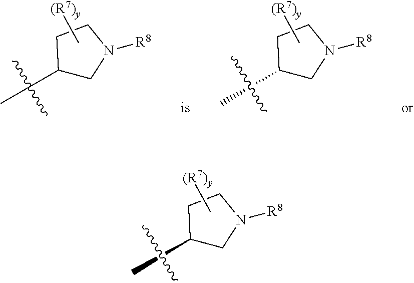

In some embodiments, as in the compounds shown in Formula I, I-3, I-3A, I-3B, I-3C, I-3D, I-3E, I-3F or I-3G, R⁷ is R⁴, which means that R⁷ and R⁴ in the compound as shown in Formula I in any of the embodiments of the present invention have the same definition.

In some embodiments, as in the compounds shown in Formula I, I-3, I-3A, I-3B, I-3C, I-3D, I-3E, I-3F or I-3G, R⁸ is R⁵, which means that R⁸ and R⁵ in the compound as shown in Formula I in any of the embodiments of the present invention have the same definition.

In some embodiments, as in the compound shown in Formula I, I-3, I-3A, I-3B, I-3C, I-3D, I-3E, I-3F, or I-3G, y is 0.

In some embodiments, as in the compounds shown in Formula I, I-3, I-3A, I-3B, I-3C, I-3D, I-3E, I-3F or I-3G, R⁸ is R⁵, —S(O)₂R^{8a} or —C(O)R^{8b}, wherein R⁵ is defined in the same way as R⁵ as defined in the compound shown in Formula I in any of the embodiments of the present invention.

In some embodiments, as in the compounds shown in Formula I, I-3, I-3A, I-3B, I-3C, I-3D, I-3E, I-3F, or I-3G, R⁸ is a —S(O)₂C(R^{a}R^{b})—C₁₋₆ alkyl group, —S(O)₂—CF₃, —S(O)₂C(R^{a}R^{b})—R^{5f}, —S(O)₂[C(R^{a}R^{b})₂]—R^{5f}, —S(O)₂[C(R^{a}R^{b})₃]—R^{5f}, —C(R^{a}R^{b})—R^{5f}, —[C(R^{a}R^{b})₂]—R_{5f}, —[C(R^{a}R^{b})₃]—R^{5f},

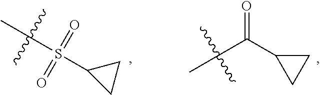 [SMILES: *S(=O)(=O)C1CC1 ; *C(=O)C1CC1]

—C(O)CH═CH₂ or —C(O)NHC(R^{a}R^{b})—R^{5f}; wherein R^{5f} is defined in the same way as R^{5f} as defined in the compound shown in Formula I in any of the embodiments of the present invention, e.g. R^{5f} is CF₃ or CN.

In some embodiments, as in the compounds shown in Formula I, I-3, I-3A, I-3D, I-3C, I-3D, I-3E, I-3F or I-3G, R⁸ is —S(O)₂C(R^{a}R^{b})—CH₃, —S(O)₂—CF₃, —C(O)NHC(R^{a}R^{b})—CF₃, —C(O)CH═CH₂,

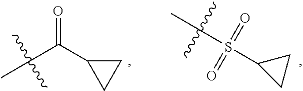 [SMILES: *C(=O)C1CC1 ; *S(=O)(=O)C1CC1]

—C(R^{a}R^{b})—CN, —[C(R^{a}R^{b})₂]—CN or —[C(R^{a}R^{b})₃]—CF₃, preferably —S(O)₂C(R^{a}R^{b})—CH₃, —S(O)₂—CF₃,

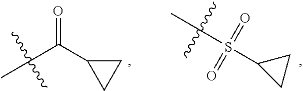 [SMILES: *C(=O)C1CC1 ; *S(=O)(=O)C1CC1]

—[C(R^{a}R^{b})₂]—CN or —[C(R^{a}R^{b})₃]—CF₃.

In some embodiments, as in the compounds shown in Formula I, I-3, I-3A, I-3B, I-3C, I-3D, I-3E, I-3F, or I-3G, R⁸ is —S(O)₂(CH₂)₂—CH₃, —S(O)₂—CF₃, —C(O)NHCH₂—CF₃, —C(O)CH═CH₂,

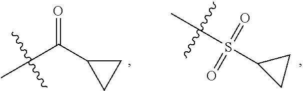 [SMILES: *C(=O)C1CC1 ; *S(=O)(=O)C1CC1]

—CH₂—CN, —(CH₂) —CN, or —(CH₂)₃—CF₃, preferably —S(O)₂(CH₂)₂—CH₃, —S(O)₂—CF₃,

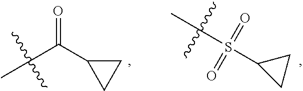 [SMILES: *C(=O)C1CC1 ; *S(=O)(=O)C1CC1]

—(CH₂)₂—CN or —(CH₂)₃—CF₃.

In some embodiments, said compound shown in Formula I has the structure shown in Formula I-4E, I-4F, I-4J, I-4L or I-4M as follows.

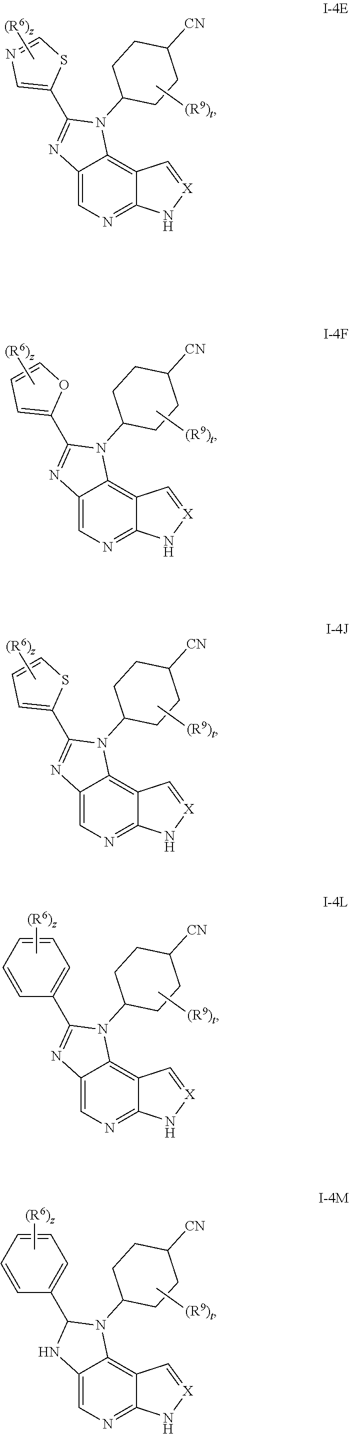

wherein X, R⁶, R⁹, z and t are defined as described in the compound shown in Formula I as defined in any of the embodiments of the present invention.

In some embodiments, compounds such as those shown in Formula I, I-4, I-4E, I-4F, I-4J, I-4L, or I-4M,

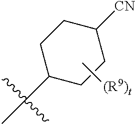

are in the cis or trans configuration, e.g., trans.

In some embodiments, as in the compounds shown in Formula I, I-4, I-4E, I-4F, I-4J, I-4L, or I-4M, t is 0.

In some embodiments, as in the compounds shown in Formula I, I-3, I-3A, I-3B, I-3C, I-3D, I-3E, I-3F, I-3G, I-4, I-4E, I-4F, I-4J, I-4L, or I-4M, X is CH.

In some embodiments, as in the compounds shown in Formula I, I-3, I-3A, I-3B, I-3C, I-3D, I-3E, I-3F, I-3G, I-4, I-4E, I-4F, I-4J, I-4L, or I-4M, X is N.

In some embodiments, as in the compounds shown in Formula I, I-3, I-3A, I-3D, I-3C, I-3D, I-3E, I-3F, I-3G, I-4, I-4E, I-4F, I-4J, I-4L, or I-4M, z is 0, 1, or 2.

In some embodiments, as in the compounds shown in Formula I, I-3, I-3A, I-3B, I-3C, I-3D, I-3E, I-3F, I-3G, I-4, I-4E, I-4F, I-4J, I-4L, or I-4M, each R⁶ is independently a halogen (e.g., fluorine), a hydroxyl group, an amino group, a cyano group, a C₁₋₄ alkyl group (e.g., methyl group), a C₁₋₄ haloalkyl group (e.g. C₁₋₄ fluoroalkyl group, e.g., CF₃), a —O—C₁₋₄ alkyl group (e.g., —O—CH₃), a —S—C₁₋₄ alkyl group (e.g., —S—CH₃), a —S(O)₂—C₁₋₄ alkyl group (e.g. —S(O)₂—CH₃), or a C₁₋₄ hydroxyalkyl group (e.g. —CH₂—OH).

In some embodiments, as in the compounds shown in Formula I, I-3, I-3A, I-3B, I-3C, I-3D, I-3E, I-3F, I-3G, I-4, I-4E, I-4F, I-4J, I-4L or I-4M, each R⁶ is independently fluorine, a hydroxyl group, an amino group, a cyano group, a methyl group, CF₃, —O—CH₃, —S—CH₃, —S(O)₂—CH₃ or —CH₂—OH.

In some embodiments, z is (in the compound as shown in Formulas I-3C.

In some embodiments, as in the compounds shown in Formula I-3D or I-4F,

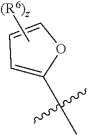

is independently

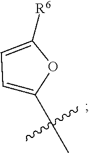

wherein R⁶ is defined as described in the compounds shown in Formula I as defined in any of the embodiments of the present invention, such as a C₁₋₄ alkyl group (e.g., methyl group) or a C₁₋₄ hydroxyalkyl group (e.g., —CH₂—OH). For example, in some embodiments,

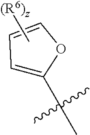

can be

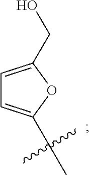 [SMILES: *c1ccc(CO)o1]

for example, in some embodiments,

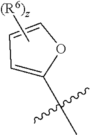

can be

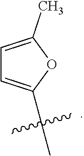 [SMILES: *c1ccc(C)o1]

In some embodiments, as in the compounds shown in Formula I-3E, I-3G, I-4L or I-4M,

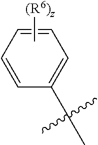

is independently

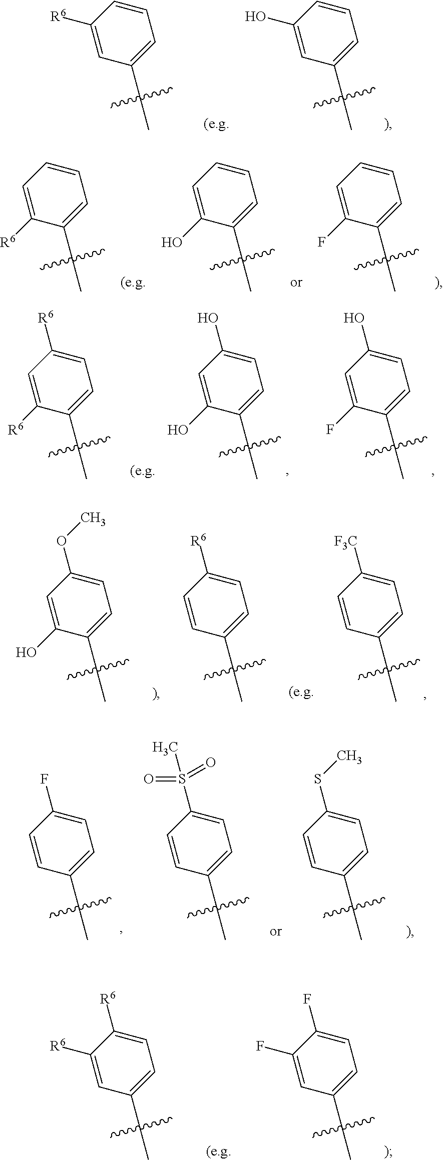

wherein each R⁶ is defined independently as described in the compounds shown in Formula I as defined in any of the embodiments of the present invention, e.g. halogen (e.g. fluorine), hydroxyl group, C₁₋₄ haloalkyl group (e.g. C₁₋₄ fluoroalkyl group, e.g. CF₃), —O—C₁₋₄ alkyl group (e.g. —O—CH₃), —S—C₁₋₄ alkyl group (e.g. —S—CH₃) or —S(O)₂—C₁₋₄ alkyl group (e.g. —S(O)₂—CH₃).

In some embodiments, as in the compound shown in Formula I-3F, is

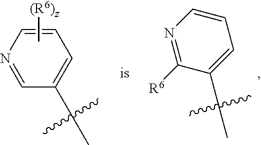

wherein R⁶ is defined as described in the compounds shown in Formula I as defined in any of the embodiments of the present invention, e.g., amino group.

In some embodiments, as in the compound shown in Formula I4E,

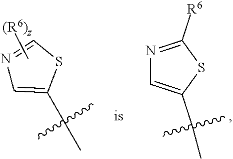

wherein R⁶ is defined as described in the compounds shown in Formula I as defined in any of the embodiments of the present invention, such as an amino group or a C₁₋₄ alkyl group (e.g., methyl group). For example, in some embodiments,

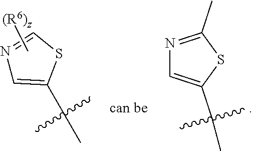

For example, in some embodiments,

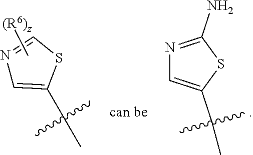

In some embodiments, as in the compound shown in Formula I-4J,

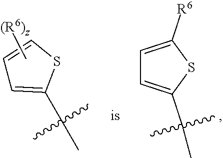

wherein R⁶ is defined as described in the compounds shown in Formula I as defined in any of the embodiments of the present invention, such as a C₁₋₄ alkyl group (e.g., methyl group). For example, in some embodiments,

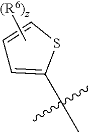

can be

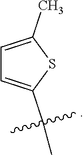 [SMILES: *c1ccc(C)s1]

In some embodiments, said compound shown in formula I has the structure shown in formula I-5 as follows.

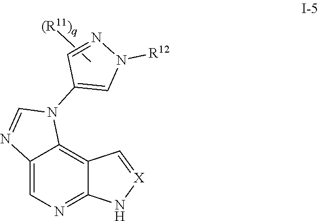

wherein X, R¹¹, R¹² and q are defined as described in the compound shown in Formula I as defined in any of the embodiments of the present invention.

In some embodiments, said compound shown in formula I has the structure shown in formula I-6 as follows.

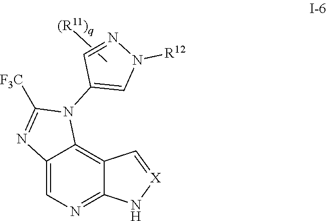

wherein X, R¹¹, R¹² and q are defined as described in the compound shown in Formula I as defined in any of the embodiments of the present invention.

In some embodiments, as in the compound shown in Formula I-5 or I-6, R¹² is independently —C(R^{e}R^{f})—CN, —[C(R^{e}R^{f})₂]—CN or —[C(R^{e}R^{f})₃]—CN, such as —CH₂—CN, —(CH₂)₂—CN, —(CH₂)₃—CN, or

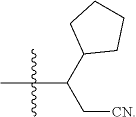 [SMILES: *C(CC#N)C1CCCC1]

In some embodiments, as in the compound shown in Formula I-5, R¹² is preferably —[C(R^{e}R^{f})₃]—CN, for example —(CH₂)₃—CN.

In some embodiments, as in the compound shown in Formula I-6, R¹² is preferably —CH₂—CN or

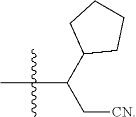 [SMILES: *C(CC#N)C1CCCC1]

In some embodiments, said compound shown in formula I has the structure shown in formula I-7 as follows.

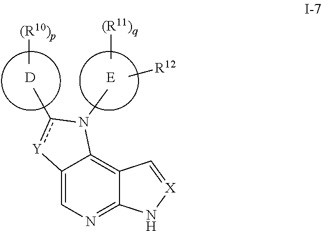

wherein X, Y, [symbol] , ring D, ring E, R¹⁰, R¹¹, R¹², p and q are defined as described in the compound shown in Formula I as defined in any of the embodiments of the present invention.

In some embodiments, as in the compound shown in Formula I or I-7, [symbol] is a double bond and Y is N.

In some embodiments, as in the compound shown in formula I or I-7 as described, [symbol] is a single bond and Y is NH.

In some embodiments, as in the compound shown in Formula I or I-7, “the 5-6-membered heteroaromatic ring” in the definition of ring D is a furan

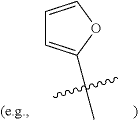 [SMILES: *c1ccco1]

or a thiophene

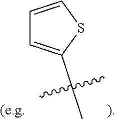 [SMILES: *c1cccs1]

In some embodiments, as in the compound shown in Formula I or I-7, ring D is a cyclopropyl group, a cyclobutyl group, a benzene ring,

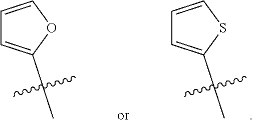 [SMILES: *c1ccco1 ; *c1cccs1]

In some embodiments, as in the compound shown in Formula I or I-7, is

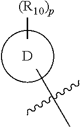

is independently

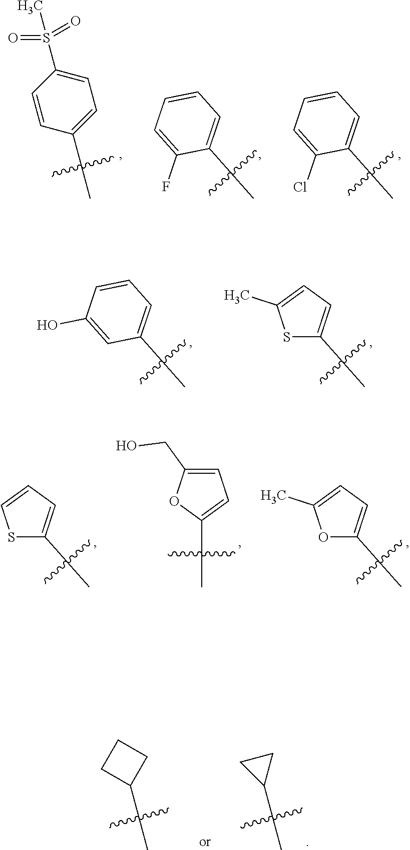

In some embodiments, said compound shown in Formula I has the structure shown in Formula I-7A, I-7B, I-7C, I-7D, I-7E or I-7F as follows.

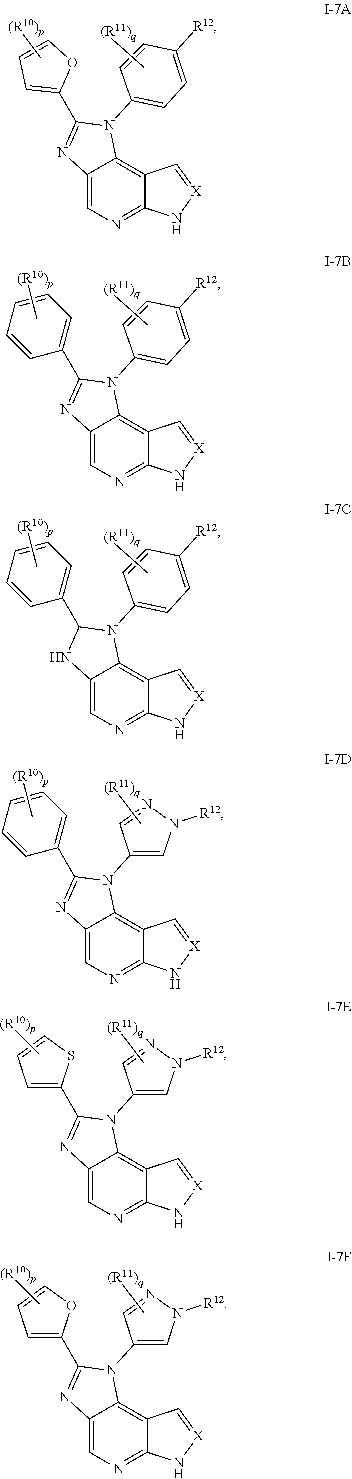

wherein X, R¹⁰, R¹¹, R¹², p and q are defined as described in any of the embodiments of the present invention.

In some embodiments, as in the compounds in Formula I, I-5, I-6, I-7, I-7A, I-7B, I-7C, I-7D, I-7E or I-7F, R¹² is a cyano group, —C(R^{e}R^{f})—R^{12f}, —[C(R^{e}R^{f})₂]—R^{12f}, —[C(R^{e}R^{f})₃]—R^{12f} or

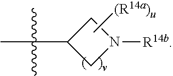

In some embodiments, as in the compounds shown in Formula I, I-5, I-6, I-7, I-7A, I-7B, I-7C, I-7D, I-7E, or I-7F, v is 0.

In some embodiments, as in the compounds shown in Formula I, I-5, I-6, I-7, I-7A, I-7B, I-7C, I-7D, I-7E, or I-7F,

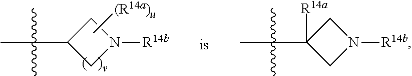

for example

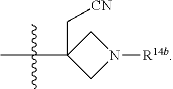

In some embodiments, as in the compounds shown in Formula I, I-5, I-6, I-7, I-7A, I-7B, I-7C, I-7D, I-7E, or I-7F, R^{14a} is a cyano-substituted C₁₋₄ alkyl group, such as —CH₂CN.

In some embodiments, as in the compounds shown in Formula I, I-5, I-6, I-7, I-7A, I-7B, I-7C, I-7D, I-7E, or I-7F, R¹² is —CN, —C(R^{e}R^{f})—CN, —[C(RR)₂]—CN, —[C(R^{e}R^{f})₃]—CN or

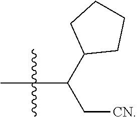 [SMILES: *C(CC#N)C1CCCC1]

In some embodiments, as in the compounds shown in Formula I, I-5, I-6, I-7, I-7A, I-7B, I-7C, I-7D, I-7E or I-7F, each R^{e} is independently H or a C₁₋₃ alkyl group.

In some embodiments, as in the compounds shown in Formula I, I-5, I-6, I-7, I-7A, I-7B, I-7C, I-7D, I-7E or I-7F, each R^{f} is independently H or a C₁₋₃ alkyl group.

In some embodiments, as in the compounds shown in Formula I, I-5, I-6, I-7, I-7A, I-7B, I-7C, I-7D, I-7E or I-7F, R¹² is —CN, —CH₂—CN, —(CH₂)₂—CN, —(CH₂)₃—CN,

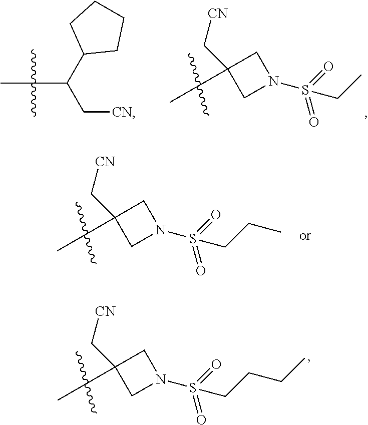 [SMILES: *C(CC#N)C1CCCC1 ; *C1(CC#N)CN(S(=O)(=O)CC)C1 ; *C1(CC#N)CN(S(=O)(=O)CCC)C1 ; *C1(CC#N)CN(S(=O)(=O)CCCC)C1]

preferably —CH₂—CN, —(CH₂)₂—CN, —(CH₂)₃—CN, further preferably —(CH₂)₂—CN.

In some embodiments, as in the compounds shown in formula I-7A, I-7B or I-7C, R¹² is a cyano group.

In some embodiments, as in the compounds shown in Formula I-7E or I-7F, R¹² is —C(R^{e}R^{f})—R^{12f}, —[C(R^{e}R^{f})₂]—R^{12f}, —C(R^{e}R^{f})₃]—R^{12f}, or

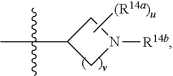

preferably —C(R^{e}R^{f})—R^{12f}, —[C(R^{e}R^{f})₂]—R^{12f} or —[C(R^{e}R^{f})₃]—R^{12f}, further preferably —[C(R^{e}R^{f})₂]—R^{12f}.

In some embodiments, said compound shown in Formula I, I-7E or I-7F has the structure shown in Formula I-7G or I-7H as follows.

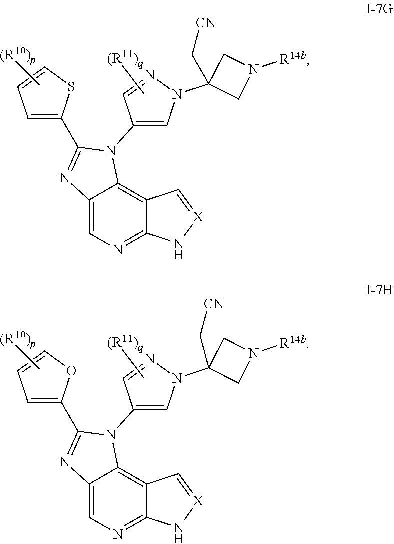

wherein X, R¹⁰, R¹¹, R^{14b}, p and q are defined as described in any of the embodiments of the present invention.

In some embodiments, as in the compounds shown in Formula I, I-5, I-6, I-7, I-7A, I-7B, I-7C, I-7D, I-7E, I-7F, I-7G or I-7H, R^{14b} is R⁵, which means that R^{14b} and R⁵ in the compound as shown in Formula I in any of the embodiments of the present invention have the same definition

In some embodiments, as in the compounds shown in Formula I, I-5, I-6, I-7, I-7A, I-7B, I-7C, I-7D, I-7E, I-7F, I-7G or I-7H, R^{14b} is a —S(O)₂—C₁₋₄ alkyl group, such as —S(O)₂—CH₂CH₃, —S(O)₂—CH₂CH₂CH₃ or —S(O)₂—CH₂CH₂CH₂CH₃.

In some embodiments, as in the compounds shown in Formula I, I-5, I-6, I-7, I-7A, I-7B, I-7C, I-7D, I-7E, I-7F, I-7G, or I-7H, X is N.

In some embodiments, as in the compounds shown in Formula I, I-5, I-6, I-7, I-7A, I-7B, I-7C, I-7D, I-7E, I-7F, 1-7G, or I-7H, X is CH.

In some embodiments, as in the compounds shown in Formula I, I-5, I-6, I-7, I-7A, I-7B, I-7C, I-7D, I-7E, I-7F, I-7G, or I-7H, q is 0.

In some embodiments, as in the compounds shown in Formula I, I-5, 1-6, I-7, I-7A, I-7B, I-7C, I-7D, I-7E, I-7F, I-7G, or I-7H, p is 0, 1, or 2.

In some embodiments, as in the compounds shown in Formula I, I-5, I-6, I-7, I-7A, I-7B, I-7C, I-7D, I-7E, I-7F, I-7G, or I-7H, each R¹⁰ is independently a halogen (e.g., fluorine or chlorine), a hydroxyl group, an amino group, a cyano group, a C₁₋₄ alkyl group (e.g., methyl group), a C₁₋₄ haloalkyl group (e.g., C₁₋₄ fluoroalkyl group, e.g., CF₃), a —O—C₁₋₄ alkyl group (e.g. —O—CH₃), a —S(O)₂—C₁₋₄ alkyl group (e.g. —S(O)₂—CH₃), or a C₁₋₄ hydroxyalkyl group (e.g. —CH₂—OH).

In some embodiments, as in the compounds shown in Formula I, I-5, I-6, I-7, I-7A, I-7B, I-7C, I-7D, I-7E, I-7F, I-7G or I-7H, each R¹⁰ is independently fluorine, chlorine, a hydroxy group, a methyl group, —S(O)₂—CH₃ or —CH₂—OH.

In some embodiments, as in the compounds shown in Formula I-7A, I-7F, or I-7H,

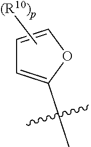

is independently,

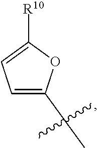

wherein each R¹⁰ is defined as described in any of the embodiments of the present invention, such as a C₁₋₄ alkyl group (e.g., methyl group) or a C₁₋₄ hydroxyalkyl group (e.g., —CH₂OH). For example, in some embodiments,

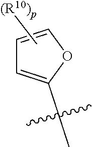

can be

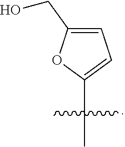 [SMILES: *c1ccc(CO)o1]

For example, in some embodiments,

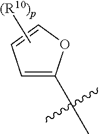

may be

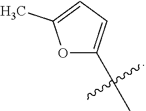 [SMILES: *c1ccc(C)o1]

In some embodiments, as in the compounds shown in Formula I-7E or I-7G,

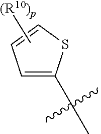

is independently

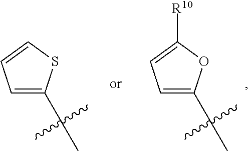

wherein each R¹⁰ is defined as described in any of the embodiments of the present invention, e.g., a C₁₋₄ alkyl group (e.g., methyl group).

In some embodiments, as in the compounds shown in Formula I-7B, I-7C or I-7D,

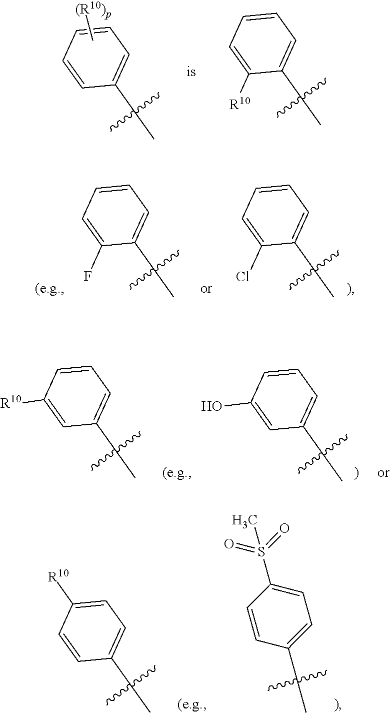

wherein each R¹⁰ is defined as described in any of the embodiments of the present invention, such as a hydroxyl group, a halogen (e.g., fluorine or chlorine) or a —S(O)₂—C₁₋₄ alkyl group (e.g. —S(O)₂—CH₃).

In some embodiments, said compound as shown in Formula I has the following structure.

| Number | Chemical name | Structure |
|---|---|---|
| LXS01 | Tert-butyl 3-(2-((R)-1- hydroxyethyl)imidazo[4,5- d]pyrrolo[2,3-b]pyridin-1(6H)- yl)pyrrolidine-1-carboxylate | 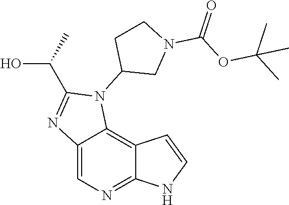 [SMILES: C[C@@H](O)c1nc2cnc3[nH]ccc3c2n1C1CCN(C(=O)OC(C)(C)C)C1] |
| LXS02 | 4-(3-(2-((R)-1- Hydroxyethyl)imidazo[4,5- d]pyrrolo[2,3-b]pyridin-1(6H)- yl)pyrrolidin-1- carbonyl)benzonitrile | 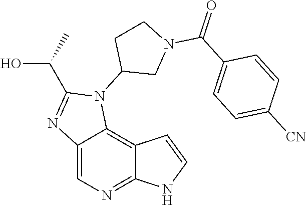 [SMILES: C[C@@H](O)c1nc2cnc3[nH]ccc3c2n1C1CCN(C(=O)c2ccc(C#N)cc2)C1] |
| LXS03 | (4-Fluorophenyl)(3-(2-((R)-1- Hydroxyethyl)imidazo[4,5- d]pyrrolo[2,3-b]pyridin-1(6H)- yl)pyrrolidin-1-yl)methanone | 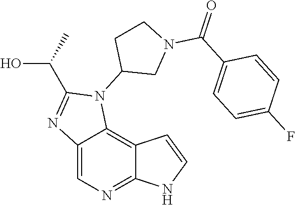 [SMILES: C[C@@H](O)c1nc2cnc3[nH]ccc3c2n1C1CCN(C(=O)c2ccc(F)cc2)C1] |
| LXS04 | (1R)-1-(1-(1- (etbylsulfonyl)pyrrolidin-3-yl)-1,6- dihydroimidazo[4,5-d]pyrrolo[2,3- b]pyridin-2-yl)ethan-1-ol | 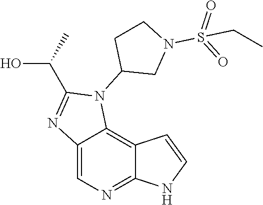 [SMILES: CCS(=O)(=O)N1CCC(n2c([C@@H](C)O)nc3cnc4[nH]ccc4c32)C1] |
| LXS05 | (1R)-1-(1-(1- (propylsulfonyl)pyrrolidin-3-yl)- 1,6-dihydroimidazo[4,5- d]pyrrolo[2,3-b]pyridin-2-yl)ethan- 1-ol | 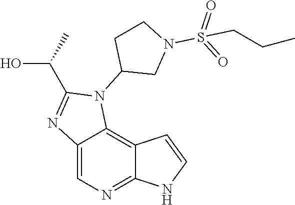 [SMILES: CCCS(=O)(=O)N1CCC(n2c([C@@H](C)O)nc3cnc4[nH]ccc4c32)C1] |
| LXS06 | (1R)-1-(1-(1- (butylsulfonyl)pyrrolidin-3-yl)-1,6- dihydroimidazo[4,5-d\|pyrrolo[2,3- b]pyridin-2-yl)ethan-1-ol | 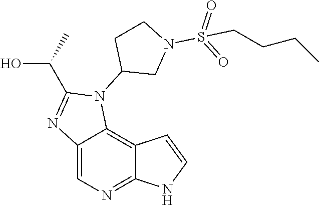 [SMILES: CCCCS(=O)(=O)N1CCC(n2c([C@@H](C)O)nc3cnc4[nH]ccc4c32)C1] |
| LXS07 | (1R)-1-(1-(1-((2- fluorophenyl)sulfonyl)pyrrolidin-3- yl)-1,6-dihydroimidazo[4,5- d]pyrrolo[2,3-b]pyridin-2-yl)ethan- 1-ol | 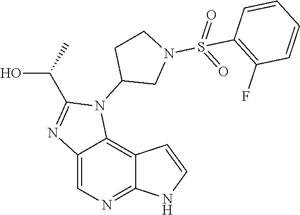 [SMILES: C[C@@H](O)c1nc2cnc3[nH]ccc3c2n1C1CCN(S(=O)(=O)c2ccccc2F)C1] |
| LXS08 | (1R)-1-(1-(1-tosylpyrrolidin-3-yl)- 1,6-dihydroimidazo[4,5- d]pyrrolo[2,3-b]pyridin-2-yl)ethan- 1-ol | 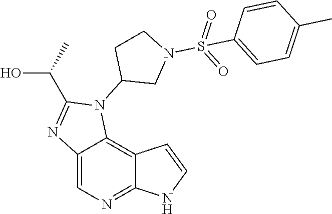 [SMILES: Cc1ccc(S(=O)(=O)N2CCC(n3c([C@@H](C)O)nc4cnc5[nH]ccc5c43)C2)cc1] |
| LXS09 | (1R)-1-(1-(1-([1,1′-biphenyl]-4- ylsulfonyl)pyrrolidin-3-yl)-1,6- dihydroimidazo[4,5-d]pyrrolo[2,3- b]pyridin-2-yl)ethan-1-ol | 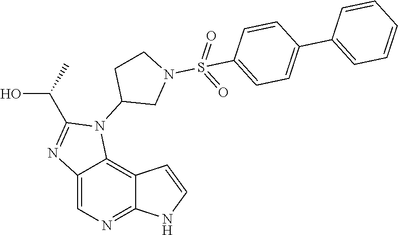 [SMILES: C[C@@H](O)c1nc2cnc3[nH]ccc3c2n1C1CCN(S(=O)(=O)c2ccc(-c3ccccc3)cc2)C1] |
| LXS10 | (1R)-1-(1-(1-(naphthalen-2- ylsulfonyl)pyrrolidin-3-yl)-1,6- dihydroimidazo[4,5-d]pyrrolo[2,3- b]pyridin-2-yl)ethan-1-ol | 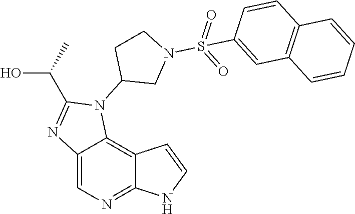 [SMILES: C[C@@H](O)c1nc2cnc3[nH]ccc3c2n1C1CCN(S(=O)(=O)c2ccc3ccccc3c2)C1] |
| LXS11 | (1R)-1-(1-(1-((4- (trifluoromethoxy)phenyl)sulfonyl) pyrrolidin-3-yl)-1,6- dihydroimidazo[4,5-d]pyrrolo[2,3- b]pyridin-2-yl)ethan-1-ol | 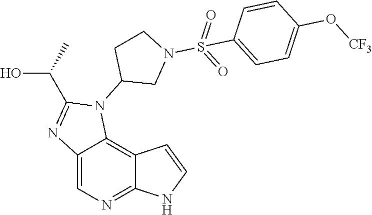 [SMILES: C[C@@H](O)c1nc2cnc3[nH]ccc3c2n1C1CCN(S(=O)(=O)c2ccc(OC(F)(F)F)cc2)C1] |
| LXS12 | (1R)-1-(1-(1-(thiophen-2- ylsulfonyl)pyrrolidin-3-yl)-1,6- dihydroimidazo[4,5-d]pyrrolo[2,3- b]pyridin-2-yl)ethan-1-ol | 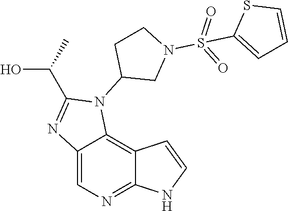 [SMILES: C[C@@H](O)c1nc2cnc3[nH]ccc3c2n1C1CCN(S(=O)(=O)c2cccs2)C1] |
| LXS13 | 2-(1-(Ethylsulfonyl)-3-(4-(2-(5- (hydroxymethyl)furan-2- yl)imidazo[4,5-d]pyrrolo[2,3- b]pyridin-1(6H)-yl)-1H-pyrazol-1- yl)azetidin-3-yl)acetonitrile | 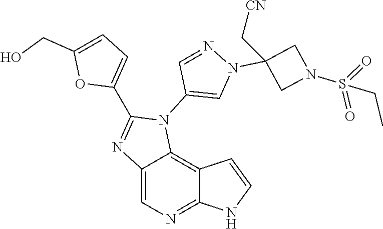 [SMILES: CCS(=O)(=O)N1CC(CC#N)(n2cc(-n3c(-c4ccc(CO)o4)nc4cnc5[nH]ccc5c43)cn2)C1] |
| LXS14 | 2-(1-(Propylsulfonyl)-3-(4-(2-(5- (hydroxymethyl)furan-2- yl)imidazo[4,5-d]pyrrolo[2,3- b]pyridin-1(6H)-yl)-1H-pyrazol-1- yl)azetidin-3-yl)acetonitrile | 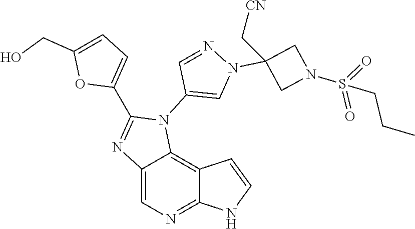 [SMILES: CCCS(=O)(=O)N1CC(CC#N)(n2cc(-n3c(-c4ccc(CO)o4)nc4cnc5[nH]ccc5c43)cn2)C1] |
| LXS15 | 2-(1-(Butylsulfonyl)-3-(4-(2-(5- (hydroxymethyl)furan-2- yl)imidazo[4,5-d]pyrrolo[2,3- b]pyridin-1(6H)-yl)-1H-pyrazol-1- yl)azetidin-3-yl)acetonitrile | 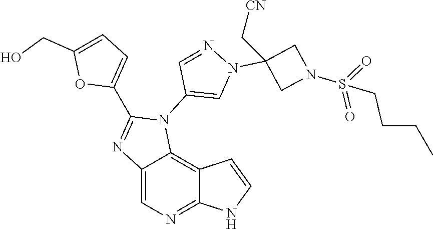 [SMILES: CCCCS(=O)(=O)N1CC(CC#N)(n2cc(-n3c(-c4ccc(CO)o4)nc4cnc5[nH]ccc5c43)cn2)C1] |
| LXS16 | (5-(1-(1-(thiophen-2- ylsulfonyl)pyrrolidin-3-yl)-1,6- dihydroimidazo[4,5-d]pyrrolo[2,3- b]pyridin-2-yl)furan-2-yl)methanol | 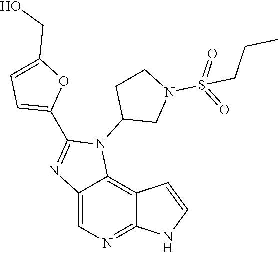 [SMILES: CCCS(=O)(=O)N1CCC(n2c(-c3ccc(CO)o3)nc3cnc4[nH]ccc4c32)C1] |
| LXS17 | Cyclopropyl(3-(2-(5- (hydroxymethyl)furan-2- yl)imidazo[4,5-d]pyrrolo[2,3- b]pyridin-1(6H)-yl)pyrrolidin-1-yl) methanone | 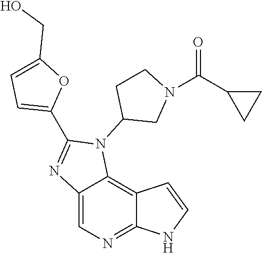 [SMILES: O=C(C1CC1)N1CCC(n2c(-c3ccc(CO)o3)nc3cnc4[nH]ccc4c32)C1] |
| LXS18 | 2-(1H-imidazol-2-yl)-1-(1- (propylsulfonyl)pyrrolidin-3-yl)- 1,6-dihydroimidazo[4,5- d]pyrrolo[2,3-b]pyridine | 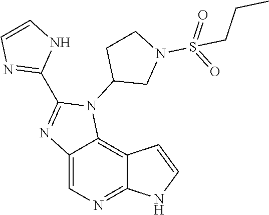 [SMILES: CCCS(=O)(=O)N1CCC(n2c(-c3ncc[nH]3)nc3cnc4[nH]ccc4c32)C1] |
| LXS19 | 2-(1H-imidazol-2-yl)-1-(1- ((trifluoromethyl)sulfonyl)pyrrolidin- 3-yl)-1,6-dihydroimidazo[4,5- d\|pyrrolo[2,3-b]pyridine | 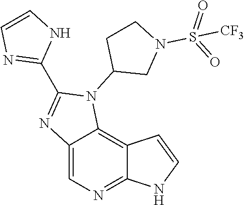 [SMILES: O=S(=O)(N1CCC(n2c(-c3ncc[nH]3)nc3cnc4[nH]ccc4c32)C1)C(F)(F)F] |
| LXS20 | (R)-3-(2-((R)-1- Hydroxyethyl)imidazo[4,5- d]pyrrolo[2,3-b]pyridin-1(6H)-yl)- N-(2,2,2-trifluoroethyl)pyrrolidine- 1-carboxamide | 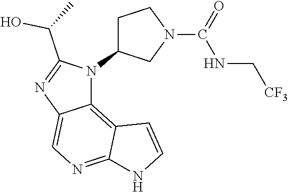 [SMILES: C[C@@H](O)c1nc2cnc3[nH]ccc3c2n1[C@H]1CCN(C(=O)NCC(F)(F)F)C1] |
| LXS21 | (R)-1-(1-((R)-1- (Propylsulfonyl)pyrrolidin-3-yl)- 1,6-dihydroimidazo[4,5- d]pyrrolo[2,3-b]pyridin-2- yl)ethanol | 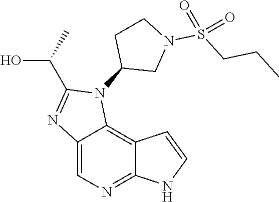 [SMILES: CCCS(=O)(=O)N1CC[C@H](n2c([C@@H](C)O)nc3cnc4[nH]ccc4c32)C1] |
| LXS22 | trans-4-(2-(5- (Hydroxymethyl)furan-2- yl)imidazo[4,5-d]pyrrolo[2,3- b]pyridin-1 (6H)- yl)cyclohexanecarbonitrile | 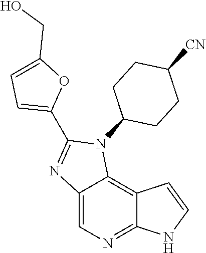 [SMILES: N#C[C@H]1CC[C@@H](n2c(-c3ccc(CO)o3)nc3cnc4[nH]ccc4c32)CC1] |
| LXS23 | (R)-3-(2-(5-(Hydroxymethyl)furan- 2-yl)imidazo[4,5-d]pyrrolo[2,3- b]pyridin-1(6H)-yl)-N-(2,2,2- trifluoroethyl)pyrrolidine-1- carboxamide | 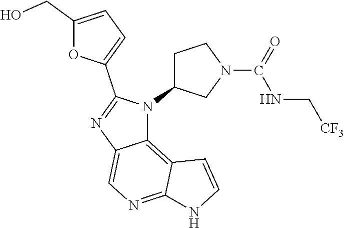 [SMILES: O=C(NCC(F)(F)F)N1CC[C@H](n2c(-c3ccc(CO)o3)nc3cnc4[nH]ccc4c32)C1] |
| LXS24 | (R)-3-(2-(1H-imidazol-2- yl)imidazo[4,5-d]pyrrolo[2,3- b]pyridin-1(6H)-yl)-N-(2,2,2- trifluoroethyl)pyrrolidine-1- carboxamide | 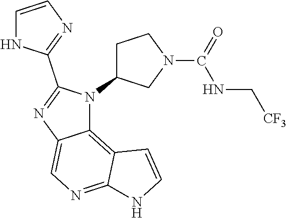 [SMILES: O=C(NCC(F)(F)F)N1CC[C@H](n2c(-c3ncc[nH]3)nc3cnc4[nH]ccc4c32)C1] |
| LXS25 | (R)-1-(3-(2-(1H-imidazol-2- yl)imidazo[4,5-d]pyrrolo[2,3- b]pyridin-1(6H)-yl)pyrrolidin-1- yl)prop-2-en-1-one | 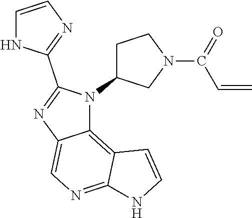 [SMILES: C=CC(=O)N1CC[C@H](n2c(-c3ncc[nH]3)nc3cnc4[nH]ccc4c32)C1] |
| LXS26 | trans-4-(2-(2-Methylthiazol-5- yl)imidazo[4,5-d]pyrrolo[2,3- b]pyridin-1(6H)- yl)cyclohexanecarbonitrile | 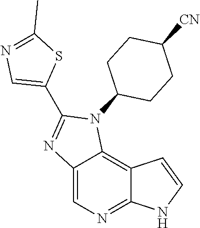 [SMILES: Cc1ncc(-c2nc3cnc4[nH]ccc4c3n2[C@H]2CC[C@@H](C#N)CC2)s1] |
| LXS27 | trans-4-(2-(2-Aminothiazol-5- yl)imidazo[4,5-d]pyrrolo[2,3- b]pyridin-1(6H)- yl)cyclohexanecarbonitrile | 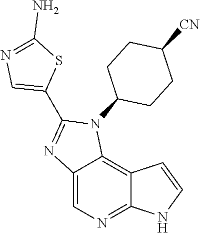 [SMILES: N#C[C@H]1CC[C@@H](n2c(-c3cnc(N)s3)nc3cnc4[nH]ccc4c32)CC1] |
| LXS28 | trans-4-(2-(2-Methylthiophen-5- yl)imidazo[4,5-d]pyrrolo[2,3- b]pyridin-1(6H)- yl)cyclohexanecarbonitrile | 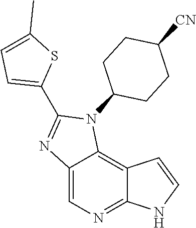 [SMILES: Cc1ccc(-c2nc3cnc4[nH]ccc4c3n2[C@H]2CC[C@@H](C#N)CC2)s1] |
| LXS29 | trans-4-(2-(2-Methylfuran-5- yl)imidazo[4,5-d]pyrrolo[2,3- b]pyridin-1(6H)- yl)cyclohexanecarbonitrile | 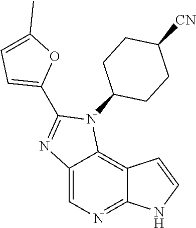 [SMILES: Cc1ccc(-c2nc3cnc4[nH]ccc4c3n2[C@H]2CC[C@@H](C#N)CC2)o1] |
| LXS30 | 2-(1-(1-(propylsulfonyl)pyrrolidin- 3-yl)-1,6-dihydroimidazo[4,5- d]pyrrolo[2,3-b]pyridin-2-yl)phenol | 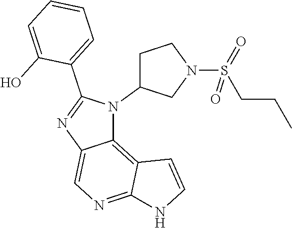 [SMILES: CCCS(=O)(=O)N1CCC(n2c(-c3ccccc3O)nc3cnc4[nH]ccc4c32)C1] |
| LXS31 | 4-(1-(1-(Propylsulfonyl)pyrrolidin- 3-yl)-1,6-dihydroimidazo[4,5- d\|pyrrolo[2,3-b]pyridin-2- yl)benzene-1,3-diol | 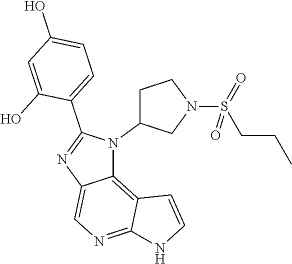 [SMILES: CCCS(=O)(=O)N1CCC(n2c(-c3ccc(O)cc3O)nc3cnc4[nH]ccc4c32)C1] |
| LXS32 | 3-(1-(1-(propylsulfonyl)pyrrolidin- 3-yl)-1,6-dihydroimidazo[4,5- d]pyrrolo[2,3-b]pyridin-2-yl)phenol | 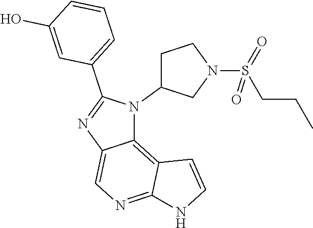 [SMILES: CCCS(=O)(=O)N1CCC(n2c(-c3cccc(O)c3)nc3cnc4[nH]ccc4c32)C1] |
| LXS33 | 1-(3-(2-(2,4- Dihydroxyphenyl)imidazo[4,5- d]pyrrolo[2,3-b]pyridin-1(6H)- yl)pyrrolidin-1-yl)prop-2-en-1-one | 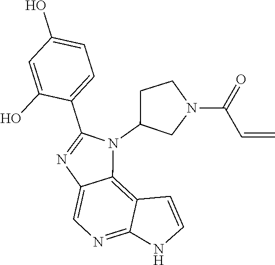 [SMILES: C=CC(=O)N1CCC(n2c(-c3ccc(O)cc3O)nc3cnc4[nH]ccc4c32)C1] |
| LXS34 | 3-(2-(3- Hydroxyphenyl)imidazo[4,5- d]pyrrolo[2,3-b]pyridin-1(6H)-yl)- N-(2,2,2-trifluoroethyl)pyrrolidine- 1-carboxamide | 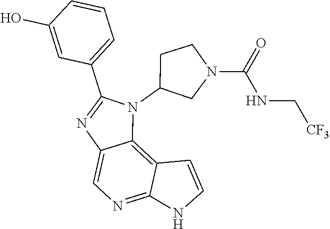 [SMILES: O=C(NCC(F)(F)F)N1CCC(n2c(-c3cccc(O)c3)nc3cnc4[nH]ccc4c32)C1] |
| LXS35 | trans-4-(2-(3- Hydroxyphenyl)imidazo[4,5- d]pyrrolo[2,3-b]pyridin-1(6H)- yl)cyclohexanecarbonitrile | 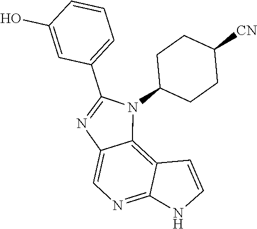 [SMILES: N#C[C@H]1CC[C@@H](n2c(-c3cccc(O)c3)nc3cnc4[nH]ccc4c32)CC1] |
| LXS36 | trans-4-(2-(2,4- Dihydroxyphenyl)imidazo[4,5- d\|pyrrolo[2,3-b]pyridin-1(6H)- yl)cyclohexanecarbonitrile | 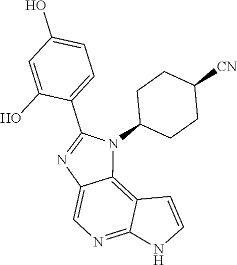 [SMILES: N#C[C@H]1CC[C@@H](n2c(-c3ccc(O)cc3O)nc3cnc4[nH]ccc4c32)CC1] |
| LXS37 | trans-4-(2-(4- (Trifluoromethyl)phenyl)imidazo[4, 5-d]pyrrolo[2,3-b]pyridin-1(6H)- yl)cyclohexanecarbonitrile | 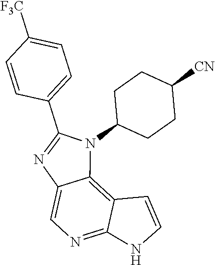 [SMILES: N#C[C@H]1CC[C@@H](n2c(-c3ccc(C(F)(F)F)cc3)nc3cnc4[nH]ccc4c32)CC1] |
| LXS38 | trans-4-(2-(2- Hydroxyphenyl)imidazo[4,5- d]pyrrolo[2,3-b]pyridin-1(6H)- yl)cyclohexanecarbonitrile | 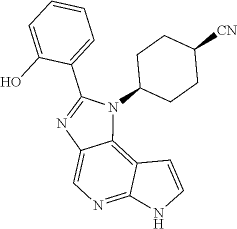 [SMILES: N#C[C@H]1CC[C@@H](n2c(-c3ccccc3O)nc3cnc4[nH]ccc4c32)CC1] |
| LXS39 | trans-4-(2-(2-Fluoro-4- hydroxyphenyl)imidazo[4,5- d]pyrrolo[2,3-b]pyridin-1(6H)- yl)cyclohexanecarbonitrile | 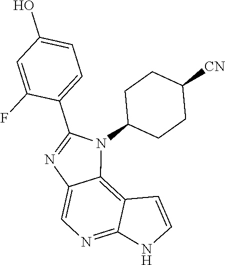 [SMILES: N#C[C@H]1CC[C@@H](n2c(-c3ccc(O)cc3F)nc3cnc4[nH]ccc4c32)CC1] |
| LXS40 | trans-4-(2-(2- Fluorophenyl)imidazo[4,5- d]pyrrolo[2,3-b]pyridin-1(6H)- yl)cyclohexanecarbonitrile | 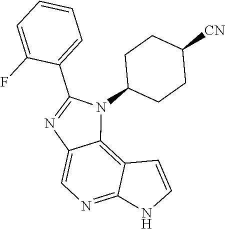 [SMILES: N#C[C@H]1CC[C@@H](n2c(-c3ccccc3F)nc3cnc4[nH]ccc4c32)CC1] |
| LXS41 | trans-4-(2-(2-Hydroxy-4- methoxyphenyl)imidazo[4,5- d\|pyrrolo[2,3-b]pyridin-1(6H)- yl)cyclohexanecarbonitrile | 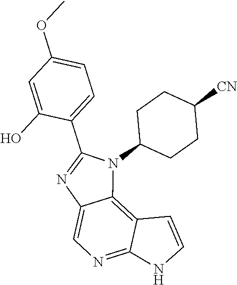 [SMILES: COc1ccc(-c2nc3cnc4[nH]ccc4c3n2[C@H]2CC[C@@H](C#N)CC2)c(O)c1] |
| LXS42 | trans-4-(2-(4- (Methylthio)phenyl)imidazo[4,5- d]pyrrolo[2,3-b]pyridin-1(6H)- yl)cyclohexanecarbonitrile | 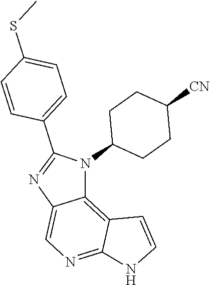 [SMILES: CSc1ccc(-c2nc3cnc4[nH]ccc4c3n2[C@H]2CC[C@@H](C#N)CC2)cc1] |
| LXS43 | trans-4-(2-(4- Fluorophenyl)imidazo[4,5- djpyrrolo[2,3-b]pyridin-1(6H)- yl)cyclohexanecarbonitrile | 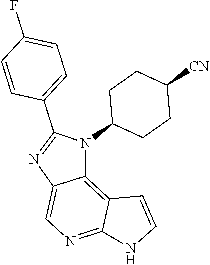 [SMILES: N#C[C@H]1CC[C@@H](n2c(-c3ccc(F)cc3)nc3cnc4[nH]ccc4c32)CC1] |
| LXS44 | trans-4-(2-(4- (Methylsulfonyl)phenyl)-2,3- dihydroimidazo[4,5-d\|pyrrolo[2,3- b]pyridin-1(6H)- yl)cyclohexanecarbonitrile | 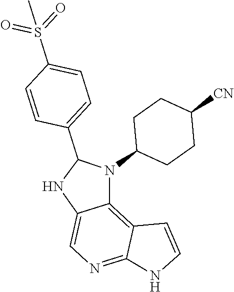 [SMILES: CS(=O)(=O)c1ccc(C2Nc3cnc4[nH]ccc4c3N2[C@H]2CC[C@@H](C#N)CC2)cc1] |
| LXS45 | (5-(1-(1- (cyclopropylsulfonyl)pyrrolidin-3- yl)-1,6-dihydroimidazo[4,5- d]pyrrolo[2,3-b]pyridin-2-yl)furan- 2-yl)methanol | 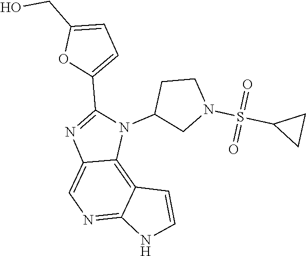 [SMILES: O=S(=O)(C1CC1)N1CCC(n2c(-c3ccc(CO)o3)nc3cnc4[nH]ccc4c32)C1] |
| LXS46 | 3-(4-(2-(5-(Hydroxymethyl)furan- 2-yl)imidazo[4,5-d]pyrrolo[2,3- b]pyridin-1(6H)-yl)-1H-pyrazol-1- yl)propanenitrile | 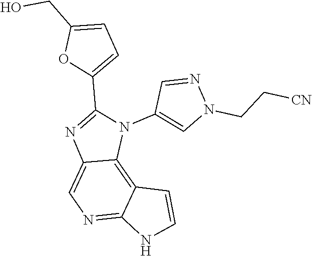 [SMILES: N#CCCn1cc(-n2c(-c3ccc(CO)o3)nc3cnc4[nH]ccc4c32)cn1] |
| LXS47 | 3-(1-(1-(propylsulfonyl)pyrrolidin- 3-yl)-1,6-dihydroimidazo[4,5- d]pyrrolo[2,3-b]pyridin-2- yl)pyridin-2-amine | 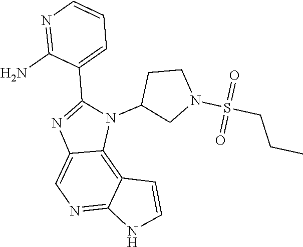 [SMILES: CCCS(=O)(=O)N1CCC(n2c(-c3cccnc3N)nc3cnc4[nH]ccc4c32)C1] |
| LXS48 | 2-(3-(2-((R)-1- Hydroxyethyl)imidazo[4,5- d]pyrrolo[2,3-b]pyridin-1(6H)- yl)pyrrolidin-1-yl)acetonitrile | 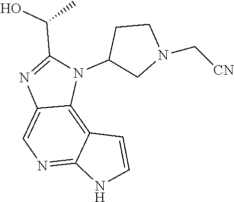 [SMILES: C[C@@H](O)c1nc2cnc3[nH]ccc3c2n1C1CCN(CC#N)C1] |
| LXS49 | 3-(3-(2-((R)-1- Hydroxyethyl)imidazo[4,5- d\|pyrrolo[2,3-b]pyridin-1(6H)- yl)pyrrolidin-1-yl)propanenitrile | 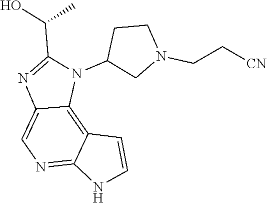 [SMILES: C[C@@H](O)c1nc2cnc3[nH]ccc3c2n1C1CCN(CCC#N)C1] |
| LXS50 | 3-(4-(2-(5-methylfuran-2- yl)imidazo[4,5-d]pyrrolo[2,3- b]pyridin-1(6H)-yl)-1H-pyrazol-1- yl)propanenitrile | 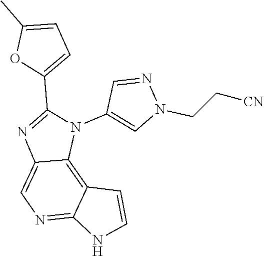 [SMILES: Cc1ccc(-c2nc3cnc4[nH]ccc4c3n2-c2cnn(CCC#N)c2)o1] |
| LXS51 | 3-(3-(2-(5-(Hydroxymethyl)furan- 2-yl)imidazo[4,5-d]pyrrolo[2,3- b]pyridin-1(6H)-yl)pyrrolidin-1- yl)propanenitrile | 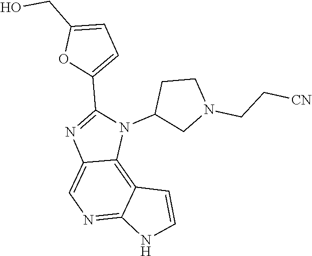 [SMILES: N#CCCN1CCC(n2c(-c3ccc(CO)o3)nc3cnc4[nH]ccc4c32)C1] |
| LXS52 | 2-(3-(2-(5-(hydroxymethyl)furan-2- yl)imidazo[4,5-d]pyrrolo[2,3- b]pyridin-1(6H)-yl)pyrrolidin-1- yl)acetonitrile | 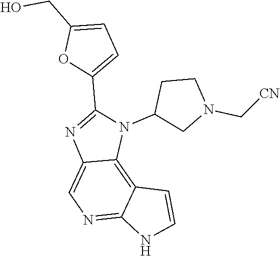 [SMILES: N#CCN1CCC(n2c(-c3ccc(CO)o3)nc3cnc4[nH]ccc4c32)C1] |
| LXS53 | 2-(3-(2-(3- Hydroxyphenyl)imidazo[4,5- d]pyrrolo[2,3-b]pyridin-1(6H)- yl)pyrrolidin-1-yl)acetonitrile | 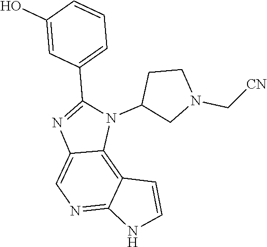 [SMILES: N#CCN1CCC(n2c(-c3cccc(O)c3)nc3cnc4[nH]ccc4c32)C1] |
| LXS54 | (R)-4-(2-(1- Hydroxyethyl)imidazo[4,5- d]pyrrolo[2,3-b]pyridin-1(6H)- yl)benzonitrile | 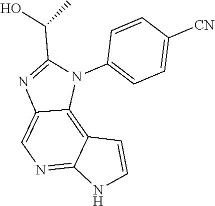 [SMILES: C[C@@H](O)c1nc2cnc3[nH]ccc3c2n1-c1ccc(C#N)cc1] |
| LXS55 | 4-(2-(5-(Hydroxymethyl)furan-2- yl)imidazo[4,5-d]pyrrolo[2,3- b]pyridin-1(6H)-yl)benzonitrile | 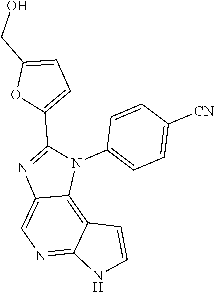 [SMILES: N#Cc1ccc(-n2c(-c3ccc(CO)o3)nc3cnc4[nH]ccc4c32)cc1] |
| LXS56 | 4-(2-(3- Hydroxyphenyl)imidazo[4,5- d]pyrrolo[2,3-b]pyridin-1(6H)- yl)benzonitrile | 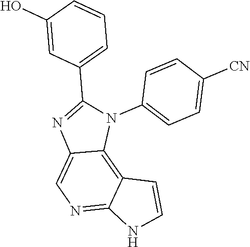 [SMILES: N#Cc1ccc(-n2c(-c3cccc(O)c3)nc3cnc4[nH]ccc4c32)cc1] |
| LXS57 | 4-(2-(4-(Methylsulfonyl)phenyl)- 2,3-dihydroimidazo[4,5- d]pyrrolo[2,3-b]pyridin-1(6H)- yl)benzonitrile | 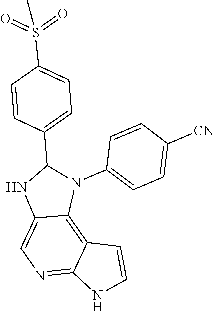 [SMILES: CS(=O)(=O)c1ccc(C2Nc3cnc4[nH]ccc4c3N2c2ccc(C#N)cc2)cc1] |
| LXS58 | 3-((R)-3-(2-((R)-1- Hydroxyethyl)imidazo[4,5- d]pyrrolo[2,3-b]pyridin-1(6H)- yl)pyrrolidin-1-yl)propanenitrile | 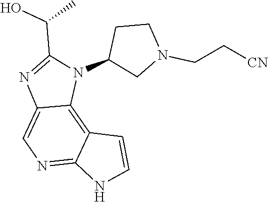 [SMILES: C[C@@H](O)c1nc2cnc3[nH]ccc3c2n1[C@H]1CCN(CCC#N)C1] |
| LXS59 | 3-((S)-3-(2-((R)-1- hydroxyethyl)imidazo[4,5- d]pyrrolo[2,3-b]pyridin-1(6H)- yl)pyrrolidin-1-yl)propanenitrile | 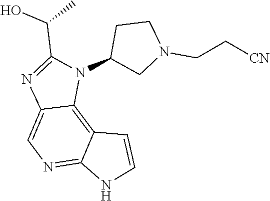 [SMILES: C[C@@H](O)c1nc2cnc3[nH]ccc3c2n1[C@H]1CCN(CCC#N)C1] |
| LXS60 | 3-(3-(2-(4- (Methylsulfonyl)phenyl)-2,3- dihydroimidazo[4,5-d]pyrrolo[2,3- b]pyridin-1(6H)-yl)pyrrolidin-1- yl)propanenitrile | 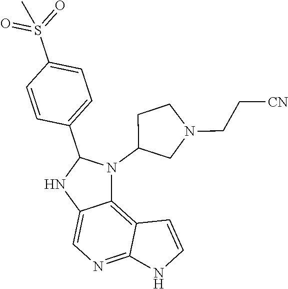 [SMILES: CS(=O)(=O)c1ccc(C2Nc3cnc4[nH]ccc4c3N2C2CCN(CCC#N)C2)cc1] |
| LXS61 | 1-(3-(2-((R)-1- Hydroxyethyl)imidazo[4,5- d]pyrrolo[2,3-b]pyridin-1(6H)- yl)pyrrolidin-1- carbonyl)cyclopropanecarbonitrile | 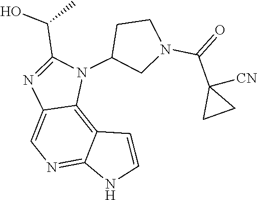 [SMILES: C[C@@H](O)c1nc2cnc3[nH]ccc3c2n1C1CCN(C(=O)C2(C#N)CC2)C1] |
| LXS62 | 2-(1-(Ethylsulfonyl)-3-(4-(2-(5- methylthien-2-yl)imidazo[4,5- d]pyrrolo[2,3-b]pyridin-1(6H)-yl)- 1H-pyrazol-1-yl)azetidin-3- yl)acetonitrile | 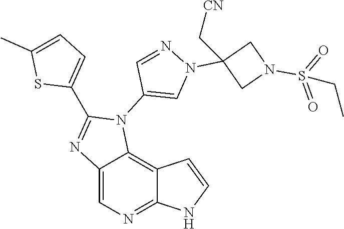 [SMILES: CCS(=O)(=O)N1CC(CC#N)(n2cc(-n3c(-c4ccc(C)s4)nc4cnc5[nH]ccc5c43)cn2)C1] |
| LXS63 | 2-(1-(Ethylsulfonyl)-3-(4-(2- (thiophen-2-yl)imidazo[4,5- d]pyrrolo[2,3-b]pyridin-1(6H)-yl)- 1H-pyrazol-1-yl)azetidin-3- yl)acetonitrile | 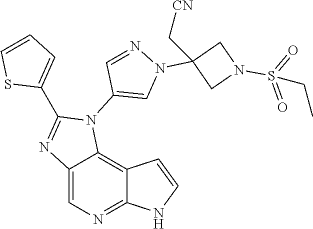 [SMILES: CCS(=O)(=O)N1CC(CC#N)(n2cc(-n3c(-c4cccs4)nc4cnc5[nH]ccc5c43)cn2)C1] |
| LXS64 | 3-(4-(2- (Trifluoromethyl)imidazo[4,5- d]pyrrolo[2,3-b]pyridin-1(6H)-yl)- 1H-pyrazol-1-yl)propanenitrile | 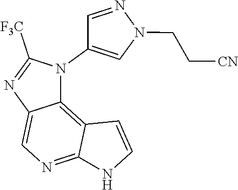 [SMILES: N#CCCn1cc(-n2c(C(F)(F)F)nc3cnc4[nH]ccc4c32)cn1] |
| LXS65 | 4-((S)-3-(2-((R)-1- Hydroxyethyl)imidazo[4,5- d]pyrrolo[2,3-b]pyridin-1(6H)- yl)pyrrolidin-1-yl)butanenitrile | 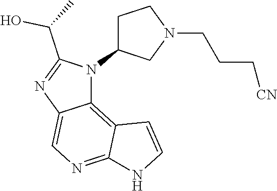 [SMILES: C[C@@H](O)c1nc2cnc3[nH]ccc3c2n1[C@H]1CCN(CCCC#N)C1] |
| LXS66 | 3-Cyclopentyl-3-(4-(2- (trifluoromethyl)imidazo[4,5- d]pyrrolo[2,3-b]pyridin-1(6H)-yl)- 1H-pyrazol-1-yl)propanenitrile | 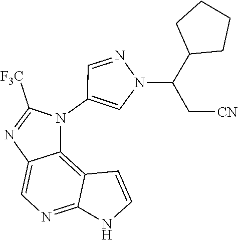 [SMILES: N#CCC(C1CCCC1)n1cc(-n2c(C(F)(F)F)nc3cnc4[nH]ccc4c32)cn1] |
| LXS67 | 3-(4-(2-(4- (Methylsulfonyl)phenyl)-2,3- dihydroimidazo[4,5-d]pyrrolo[2,3- b]pyridin-1(6H)-yl)-1H-pyrazol-1- yl)propanenitrile | 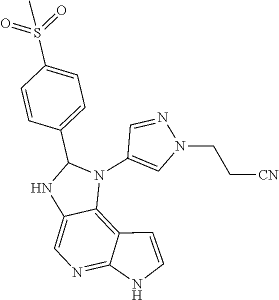 [SMILES: CS(=O)(=O)c1ccc(C2Nc3cnc4[nH]ccc4c3N2c2cnn(CCC#N)c2)cc1] |
| LXS68 | 5-((S)-3-(2-((R)-1- Hydroxyethyl)imidazo[4,5- d]pyrrolo[2,3-b]pyridin-1(6H)- yl)pyrrolidin-1-yl)pentanonitrile | 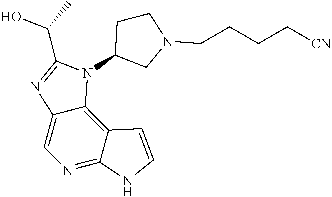 [SMILES: C[C@@H](O)c1nc2cnc3[nH]ccc3c2n1[C@H]1CCN(CCCCC#N)C1] |
| LXS69 | 4-((S)-7-(2-((R)-1- Hydroxyethyl)imidazo[4,5- d]pyrrolo[2,3-b]pyridin-1(6H)-yl)- 5-azaspiro[2.4]hept-5- yl)butanenitrile | 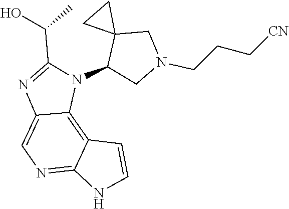 [SMILES: C[C@@H](O)c1nc2cnc3[nH]ccc3c2n1[C@@H]1CN(CCCC#N)CC12CC2] |
| LXS70 | (R)-1-(1-((S)-5-(4,4,4- Trifluorobutyl)-5- azaspiro[2.4]hept-7-yl)-1,6- dihydroimidazo[4,5-d ]pyrrolo[2,3- b]pyridin-2-yl)ethanol | 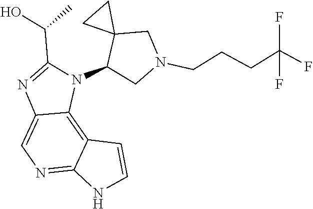 [SMILES: C[C@@H](O)c1nc2cnc3[nH]ccc3c2n1[C@@H]1CN(CCCC(F)(F)F)CC12CC2] |
| LXS71 | (R)-1-(1-((S)-1-(4,4,4- Trifluorobutyl)pyrrolidin-3-yl)-1,6- dihydroimidazo[4,5-d]pyrrolo[2,3- b]pyridin-2-yl)ethanol | 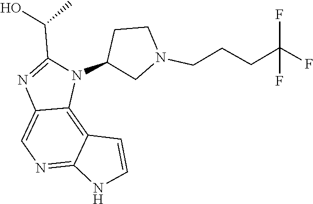 [SMILES: C[C@@H](O)c1nc2cnc3[nH]ccc3c2n1[C@H]1CCN(CCCC(F)(F)F)C1] |
| LXS72 | 5-((S)-7-(2-((R)-1- Hydroxyethyl)imidazo[4,5- d]pyrrolo[2,3-b]pyridin-1(6H)-yl)- 5-azaspiro[2.4]hept-5- yl)pentanonitrile | 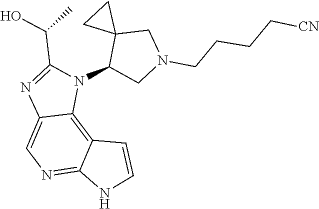 [SMILES: C[C@@H](O)c1nc2cnc3[nH]ccc3c2n1[C@@H]1CN(CCCCC#N)CC12CC2] |
| LXS73 | (R)-1-(1-((S)-1-(5- Fluoropentyl)pyrrolidin-3-yl)-1,6- dihydroimidazo[4,5-d]pyrrolo[2,3- b]pyridin-2-yl)ethanol | 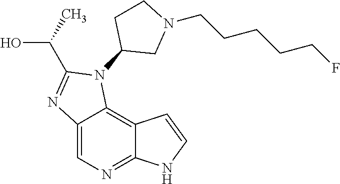 [SMILES: C[C@@H](O)c1nc2cnc3[nH]ccc3c2n1[C@H]1CCN(CCCCCF)C1] |
| LXS74 | 2-((S)-3-(2-((R)-1- Hydroxyethyl)imidazo[4,5- d]pyrrolo[2,3-b]pyridin-1(6H)- yl)pyrrolidin-1-yl)-N-(2,2,2- trifluoroethyl)acetamide | 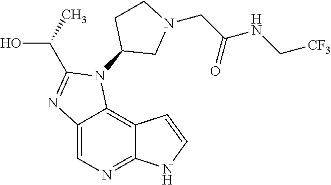 [SMILES: C[C@@H](O)c1nc2cnc3[nH]ccc3c2n1[C@H]1CCN(CC(=O)NCC(F)(F)F)C1] |
| LXS75 | 2-((S)-3-(2-((R)-1- Hydroxyethyl)imidazo[4,5- d]pyrrolo[2,3-b]pyridin-1(6H)- yl)pyrrolidin-1-yl)-N-(2,2,2- trifluoroethyl)propanamide | 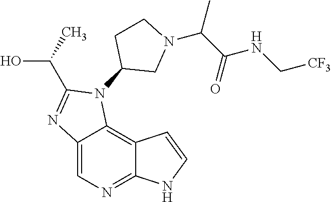 [SMILES: CC(C(=O)NCC(F)(F)F)N1CC[C@H](n2c([C@@H](C)O)nc3cnc4[nH]ccc4c32)C1] |
| LXS76 | 2-((S)-3-(2-((R)-1- Hydroxyethyl)imidazo[4,5- d]pyrrolo[2,3-b]pyridin-1(6H)- yl)pyrrolidin-1-yl)-N-(2,2,2- trifluoroethyl)butanamide | 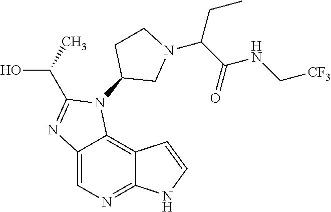 [SMILES: CCC(C(=O)NCC(F)(F)F)N1CC[C@H](n2c([C@@H](C)O)nc3cnc4[nH]ccc4c32)C1] |
| LXS77 | 2-(1-(ethylsulfonyl)-3-(4-(2-(5- methylfuran-2-yl)imidazo[4,5- d]pyrrolo[2,3-b]pyridin-1(6H)-yl)- 1H-pyrazol-1-yl)azetidin-3- yl)acetonitrile | 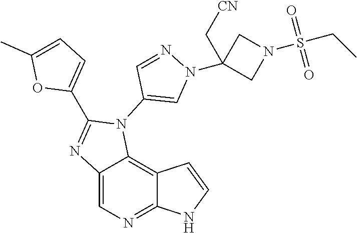 [SMILES: CCS(=O)(=O)N1CC(CC#N)(n2cc(-n3c(-c4ccc(C)o4)nc4cnc5[nH]ccc5c43)cn2)C1] |
| LXS78 | 2-(4-(2- (Trifluoromethyl)imidazo[4,5- d]pyrrolo[2,3-b]pyridin-1(6H)-yl)- 1H-pyrazol-1-yl)acetonitrile | 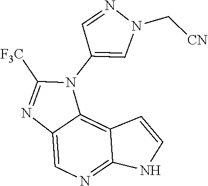 [SMILES: N#CCn1cc(-n2c(C(F)(F)F)nc3cnc4[nH]ccc4c32)cn1] |
| LXS79 | 3-(4-(Imidazo[4,5-d]pyrrolo[2,3- b]pyridin-1(6H)-yl)-1H-pyrazol-1- yl)propanenitrile | 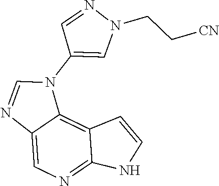 [SMILES: N#CCCn1cc(-n2cnc3cnc4[nH]ccc4c32)cn1] |
| LXS80 | 3-(4-(2-(2- Chlorophenyl)imidazo[4,5- d]pyrrolo[2,3-b]pyridin-1(6H)-yl)- 1H-pyrazol-1-yl)propanenitrile | 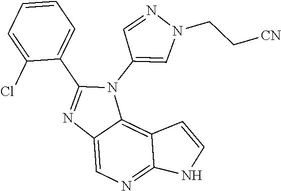 [SMILES: N#CCCn1cc(-n2c(-c3ccccc3Cl)nc3cnc4[nH]ccc4c32)cn1] |
| LXS81 | 2-(4-(2-(5-(Hydroxymethyl)furan- 2-yl)imidazo[4,5-d]pyrrolo[2,3- b]pyridin-1(6H)-yl)-1H-pyrazol-1- yl)acetonitrile | 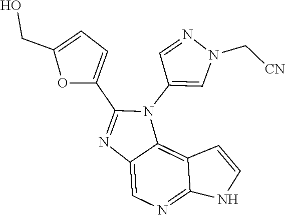 [SMILES: N#CCn1cc(-n2c(-c3ccc(CO)o3)nc3cnc4[nH]ccc4c32)cn1] |
| LXS82 | 3-(4-(2-(3- Hydroxyphenyl)imidazo[4,5- d]pyrrolo[2,3-b]pyridin-1(6H)-yl)- 1H-pyrazol-1-yl)propanenitrile | 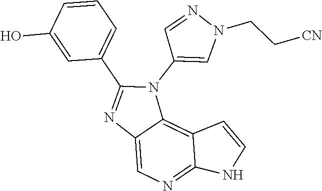 [SMILES: N#CCCn1cc(-n2c(-c3cccc(O)c3)nc3cnc4[nH]ccc4c32)cn1] |
| LXS83 | 3-(4-(2-(2- Fluorophenyl)imidazo[4,5- d]pyrrolo[2,3-b]pyridin-1(6H)-yl)- 1H-pyrazol-1-yl)propanenitrile | 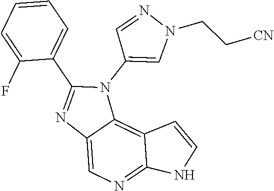 [SMILES: N#CCCn1cc(-n2c(-c3ccccc3F)nc3cnc4[nH]ccc4c32)cn1] |
| LXS84 | 3-(4-(2-(5-methylthiophen-2- yl)imidazo[4,5-d]pyrrolo[2,3- b]pyridin-1(6H)-yl)-1H-pyrazol-1- yl)propanenitrile | 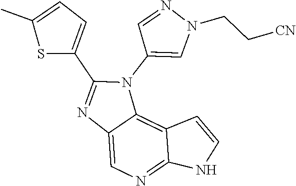 [SMILES: Cc1ccc(-c2nc3cnc4[nH]ccc4c3n2-c2cnn(CCC#N)c2)s1] |
| LXS85 | 4-((S)-3-(2-((R)-1- Hydroxyethyl)imidazo[4,5- d]pyrazolo[3,4-b]pyridin-1(6H)- yl)pyrrolidin-1-yl)butanenitrile | 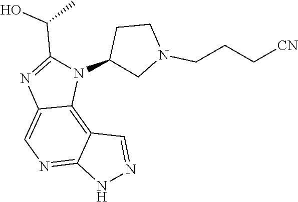 [SMILES: C[C@@H](O)c1nc2cnc3[nH]ncc3c2n1[C@H]1CCN(CCCC#N)C1] |
| LXS86 | (1R)-1-(1-((3R)-4-fluoro-1-(4,4,4- trifluorobutyl)pyrrolidin-3-yl)-1,6- dihydroimidazo[4,5-d]pyrrolo[2,3- b]pyridin-2-yl)ethan-1-ol | 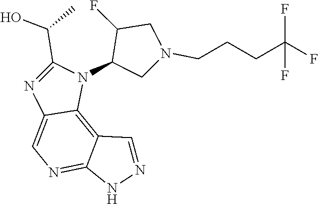 [SMILES: C[C@@H](O)c1nc2cnc3[nH]ncc3c2n1[C@@H]1CN(CCCC(F)(F)F)CC1F] |
| LXS87 | (R)-1-(1-((3R,4R)-4-fluoro-1- (4,4,4-trifluorobutyl)pyrrolidin-3- yl)-1,6-dihydroimidazo[4,5- d]pyrrolo[2,3-b]pyridin-2-yl)ethan- 1-ol | 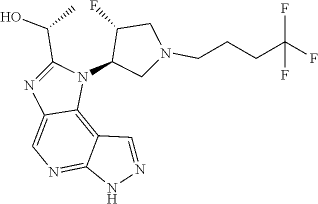 [SMILES: C[C@@H](O)c1nc2cnc3[nH]ncc3c2n1[C@@H]1CN(CCCC(F)(F)F)C[C@H]1F] |
| LXS88 | (R)-1-(1-((3R,4S)-4-fluoro-1- (4,4,4-trifluorobutyl)pyrrolidin-3- yl)-1,6-dihydroimidazo[4,5- d]pyrrolo[2,3-b]pyridin-2-yl)ethan- 1-ol | 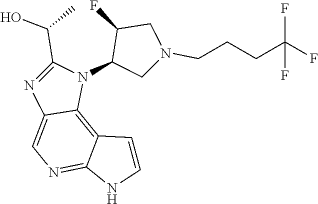 [SMILES: C[C@@H](O)c1nc2cnc3[nH]ccc3c2n1[C@@H]1CN(CCCC(F)(F)F)C[C@@H]1F] |
| LXS89 | (R)-1-(1-((38,5S)-5- (hydroxymethyl)-1-(4,4,4- trifluorobutyl)pyrrolidin-3-yl)-1,6- dihydroimidazo[4,5-d]pyrrolo[2,3- b]pyridin-2-yl)ethan-1-ol | 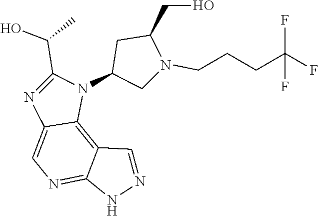 [SMILES: C[C@@H](O)c1nc2cnc3[nH]ncc3c2n1[C@H]1C[C@@H](CO)N(CCCC(F)(F)F)C1] |
| LXS90 | (R)-1-(1-((S)-1-(4,4,4- Trifluorobutyl)pyrrolidin-3-yl)-1,6- dihydroimidazo[4,5- d]pyrazolo[3,4-b]pyridin-2- yl)ethanol | 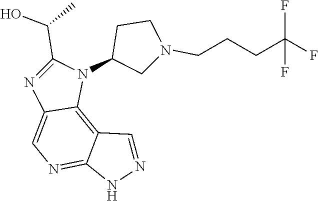 [SMILES: C[C@@H](O)c1nc2cnc3[nH]ncc3c2n1[C@H]1CCN(CCCC(F)(F)F)C1] |
| LXS91 | (R)-1-(1-((3S,5R)-5- (hydroxymethyl)-1-(4,4,4- trifluorobutyl)pyrrolidin-3-yl)-1,6- dihydroimidazo[4,5-d]pyrrolo[2,3- b]pyridin-2-yl)ethan-1-ol | 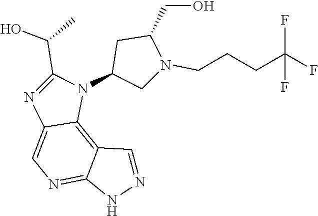 [SMILES: C[C@@H](O)c1nc2cnc3[nH]ncc3c2n1[C@H]1C[C@H](CO)N(CCCC(F)(F)F)C1] |
| LXS92 | (R)-1-(1-((38,5R)-5- (hydroxymethyl)-1-(4,4,4- trifluorobutyl)pyrrolidin-3-yl)-1,6- dihydroimidazo[4,5-d]pyrrolo[2,3- b]pyridin-2-yl)ethan-1-ol | 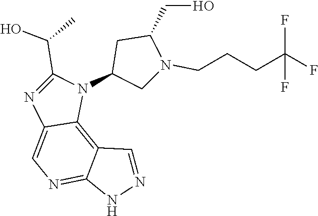 [SMILES: C[C@@H](O)c1nc2cnc3[nH]ncc3c2n1[C@H]1C[C@H](CO)N(CCCC(F)(F)F)C1] |
| LXS93 | 4-((4R)-3-Fluoro-4-(2-((R)-1- hydroxyethyl)imidazo[4,5- d]pyrazolo[3,4-b]pyridin-1(6H)-yl) pyrrolidin-1-yl)butanenitrile | 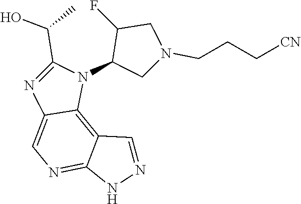 [SMILES: C[C@@H](O)c1nc2cnc3[nH]ncc3c2n1[C@@H]1CN(CCCC#N)CC1F] |
| LXS94 | 4-((2S,4S)-4-(2-((R)-1- Hydroxyethyl)imidazo[4,5- d]pyrazolo[3,4-b]pyridin-1(6H)-yl)- 2-(hydroxymethyl)pyrrolidin-1- yl)butanenitrile | 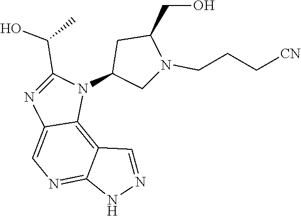 [SMILES: C[C@@H](O)c1nc2cnc3[nH]ncc3c2n1[C@H]1C[C@@H](CO)N(CCCC#N)C1] |
| LXS95 | trans-4-(2-(3,4- Difluorophenyl)imidazo[4,5- d]pyrrolo[2,3-b]pyridin-1(6H)- yl)cyclohexanecarbonitrile | 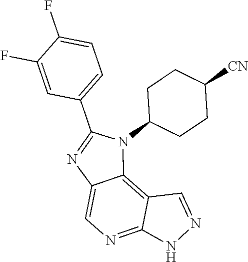 [SMILES: N#C[C@H]1CC[C@@H](n2c(-c3ccc(F)c(F)c3)nc3cnc4[nH]ncc4c32)CC1] |
| LXS96 | (R)-1-(1-((S)-1-(3,3,3- Trifluoropropyl)pyrrolidin-3-yl)- 1,6-dihydroimidazo[4,5- d]pyrrolo[2,3- b]pyridin-2- yl)ethanol | 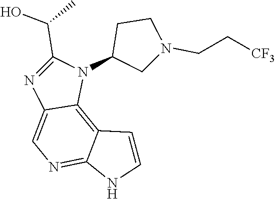 [SMILES: C[C@@H](O)c1nc2cnc3[nH]ccc3c2n1[C@H]1CCN(CCC(F)(F)F)C1] |
| LXS97 | (R)-1-(1-((S)-1-(3,3,4,4,4- Pentafluorobutyl)pyrrolidin-3-yl)- 1,6-dihydroimidazo[4,5- d]pyrrolo[2,3-b]pyridin-2- yl)ethanol | 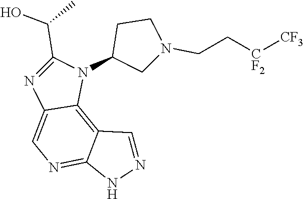 [SMILES: C[C@@H](O)c1nc2cnc3[nH]ncc3c2n1[C@H]1CCN(CCC(F)(F)C(F)(F)F)C1] |
| LXS98 | (S)-(5-(1-(1-(4,4,4- trifluorobutyl)pyrrolidin-3-yl)-1,6- dihydroimidazo[4,5-d]pyrrolo[2,3- b]pyridin-2-yl)furan-2-yl)methanol | 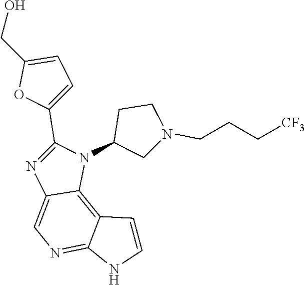 [SMILES: OCc1ccc(-c2nc3cnc4[nH]ccc4c3n2[C@H]2CCN(CCCC(F)(F)F)C2)o1] |
| LXS99 | (S)-3-(1-(1-(4,4,4- trifluorobutyl)pyrrolidin-3-yl)-1,6- dihydroimidazo[4,5-d]pyrrolo[2,3- b]pyridin-2-yl)phenol | 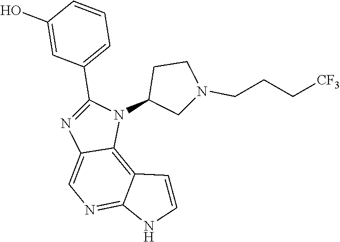 [SMILES: Oc1cccc(-c2nc3cnc4[nH]ccc4c3n2[C@H]2CCN(CCCC(F)(F)F)C2)c1] |
| LXS100 | (R)-1-(1-((S)-1-(2,2,3,3,4,4,4- heptafluorobutyl)pyrrolidin-3-yl)- 1,6-dihydroimidazo[4,5- d]pyrrolo[2,3-b]pyridin-2- yl)ethanol | 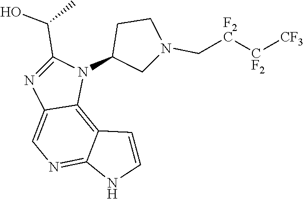 [SMILES: C[C@@H](O)c1nc2cnc3[nH]ccc3c2n1[C@H]1CCN(CC(F)(F)C(F)(F)C(F)(F)F)C1] |
| LXS101 | (R)-1-(1-((S)-1-(Perfluoro- butyl)pyrrolidin-3-yl)-1,6- dihydroimidazo[4,5-d]pyrrolo[2,3- b]pyridin-2-yl)ethanol | 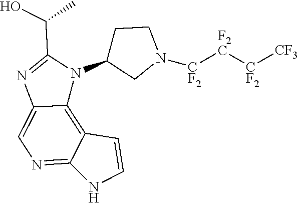 [SMILES: C[C@@H](O)c1nc2cnc3[nH]ccc3c2n1[C@H]1CCN(C(F)(F)C(F)(F)C(F)(F)C(F)(F)F)C1] |
| LXS102 | (R)-1-(1-((S)-1-((3,3,3- Trifluoropropyl)sulfonyl)pyrrolidin- 3-yl)-1,6-dihydroimidazo[4,5- d\|pyrrolo[2,3-b]pyridin-2- yl)ethanol | 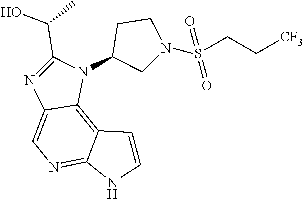 [SMILES: C[C@@H](O)c1nc2cnc3[nH]ccc3c2n1[C@H]1CCN(S(=O)(=O)CCC(F)(F)F)C1] |
| LXS103 | 4-(4-(Imidazo[4,5-d]pyrrolo[2,3- b]pyridin-1(6H)-yl)-1H-pyrazol-1- yl)butanenitrile | 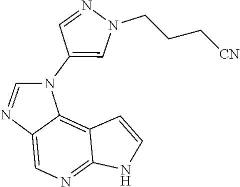 [SMILES: N#CCCCn1cc(-n2cnc3cnc4[nH]ccc4c32)cn1] |
| LXS104 | 4-(4-(2-(5-methylfuran-2- yl)imidazo[4,5-d]pyrrolo[2,3- b]pyridin-1(6H)-yl)-1H-pyrazol-1- yl)butanenitrile | 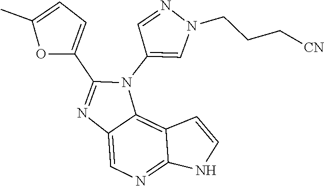 [SMILES: Cc1ccc(-c2nc3cnc4[nH]ccc4c3n2-c2cnn(CCCC#N)c2)o1] |
| LXS105 | 4-(4-(2-(5-Methylthiophen-2- yl)imidazo[4,5-d]pyrrolo[2,3- b]pyridin-1(6H)-yl)-1H-pyrazol-1- yl butanenitrile | 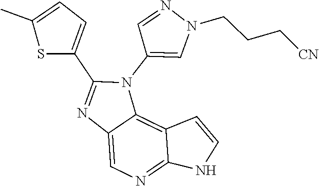 [SMILES: Cc1ccc(-c2nc3cnc4[nH]ccc4c3n2-c2cnn(CCCC#N)c2)s1] |
| LXS106 | 3-(4-(2-(Thiophen-2- yl)imidazo[4,5-d]pyrrolo[2,3- b]pyridin-1(6H)-yl)-1H-pyrazol-1- yl)propanenitrile | 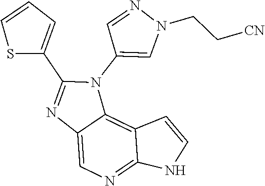 [SMILES: N#CCCn1cc(-n2c(-c3cccs3)nc3cnc4[nH]ccc4c32)cn1] |
| LXS107 | 4-(4-(2-Cyclobutylimidazo[4,5- d]pyrrolo[2,3-b]pyridin-1(6H)-yl)- 1H-pyrazol-1-yl)butanenitrile | 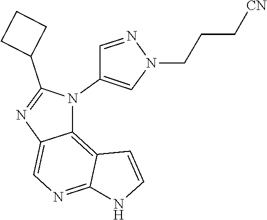 [SMILES: N#CCCCn1cc(-n2c(C3CCC3)nc3cnc4[nH]ccc4c32)cn1] |
| LXS108 | 4-(4-(2-Cyclopropylimidazo[4,5- d]pyrrolo[2,3-b]pyridin-1(6H)-yl)- 1H-pyrazol-1-yl)butanenitrile | 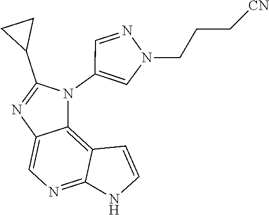 [SMILES: N#CCCCn1cc(-n2c(C3CC3)nc3cnc4[nH]ccc4c32)cn1] |

On the other hand, the present invention also provides a method for the preparation of a compound as shown in formula I, which includes the following steps: reacting a compound as shown in formula 1I with a base (e.g. sodium hydroxide) in an organic solvent (e.g. a mixture of tetrahydrofuran and methanol) to obtain said compound as shown in formula I.

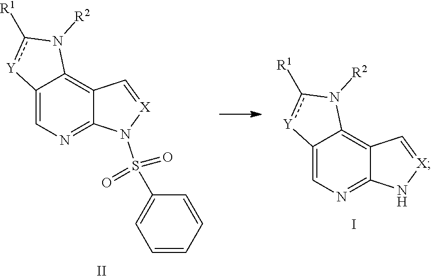

wherein X, Y, [symbol] , R¹ and R² are defined as described in any of the embodiments of the present invention.

On the other hand, the present invention also provides a compound as shown in Formula 1I.

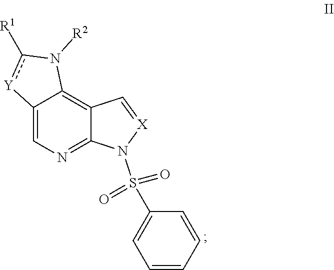

wherein X, Y, [symbol] , R¹ and R² are defined as described in any of the embodiments of the present invention.

On the other hand, the present invention also provides a pharmaceutical composition comprising:
said compound shown in Formula I, or a tautomer, stereoisomer, racemate or isotopic derivative thereof, or a pharmaceutically acceptable salt of any of the foregoing, or a crystalline or solvated form of any of the foregoing; and
(ii) a pharmaceutically acceptable vehicle.

On the other hand, the present invention also provides a compound as described in Formula I, or a tautomer, stereoisomer, racemate or isotopic derivative thereof, or a pharmaceutically acceptable salt of any of the foregoing, or a crystalline or solvated form of any of the foregoing, or an application of said pharmaceutical composition as a drug.

On the other hand, the present invention also provides a compound as described in Formula I, or a tautomer, stereoisomer, racemate or isotopic derivative thereof, or a pharmaceutically acceptable salt of any of the foregoing, or a crystalline or solvated form of any of the foregoing, or an application of said pharmaceutical composition which acts as an inhibitor of Janus kinase (e.g., JAK1 and/or JAK2).

On the other hand, the present invention also provides a method of inhibiting Janus kinase (e.g. JAK1 and/or JAK2) in vivo, in vitro or ex vivo, which includes the contact of said compound shown in Formula I, or a tautomer, stereoisomer, racemate or isotopic derivative thereof, or a pharmaceutically acceptable salt of any of the foregoing, or a crystalline or solvated form of any of the foregoing, or said pharmaceutical composition with said Janus kinase.

On the other hand, the present invention also provides a compound as described in Formula I, or a tautomer, stereoisomer, racemate or isotopic derivative thereof, or a pharmaceutically acceptable salt of any of the foregoing, or a crystalline or solvated form of any of the foregoing, or a pharmaceutical composition as described in the preparation of drugs for the treatment of diseases associated with Janus kinases (e.g. JAK1 and/or JAK2).

On the other hand, the present invention also provides a compound as shown in Formula I, or a tautomer, stereoisomer, racemate or isotopic derivative thereof, or a pharmaceutically acceptable salt of any of the foregoing, or a crystalline or solvated form of any of the foregoing, or an application of said pharmaceutical composition in the preparation of a drug for the treatment of an autoimmune disease or cancer.

On the other hand, the present invention also provides a method of treating a disease associated with a Janus kinase (e.g., JAK1 and/or JAK2) which includes administering a therapeutically effective amount of said compound shown in Formula I, or a tautomer, stereoisomer, racemate or isotopic derivative thereof, or a pharmaceutically acceptable salt of any of the foregoing, or a crystalline or solvated form of any of the foregoing, or a pharmaceutical composition as described to a subject in need of such treatment.

On the other hand, the present invention also provides a method of treating an autoimmune disease or cancer which includes administering a therapeutically effective amount of said compound shown in Formula I, or a tautomer, stereoisomer, racemate or isotopic derivative thereof, or a pharmaceutically acceptable salt of any of the foregoing, or a crystalline or solvated form of any of the foregoing, or a pharmaceutical composition as described to a subject in need of such treatment.

The diseases associated with Janus kinases (e.g., JAK1 and/or JAK2) described in the present invention can be autoimmune diseases or cancers.

The autoimmune diseases described herein or autoimmune diseases associated with Janus kinase can be, for example, psoriasis, rheumatoid arthritis, inflammatory bowel disease, Sjogren's syndrome, leukoaraiosis, multiple sclerosis, or systemic lupus erythematosus.

The cancer or cancers associated with Janus kinase described in the present invention may be, for example, Kapozi's sarcoma, giant lymph node hyperplasia, lymphoma, leukemia multiple myeloma, or myeloproliferative disorders. Myeloproliferative disorders described may be, for example, polycythemia vera (PV), essential thrombocytosis (ET), primary myelofibrosis (PMF), chronic myeloid leukemia (CML), chronic monocytic leukemia (CMML), eosinophilic leukocytosis syndrome (HES), idiopathic myelofibrosis (IMF), or systemic mastocytosis (SMCD).

Unless otherwise stated, terms used in the present invention have the following definitions, and terms not covered below are defined as common knowledge understood by those skilled in the art of the present invention.

The term “tautomer” refers to a functional group isomer that results from the rapid movement of an atom in two positions in a molecule. For example, acetone and 1-propen-2-ol can transform into each other through the rapid movement of the hydrogen atom between the oxygen atom and a -carbon.

The term “stereoisomers” refers to isomers of molecules in which atoms or groups of atoms are attached to each other in the same order but are in different spatial arrangements, such as cis-trans isomers (e.g., Z-isomer, E-isomer), optical isomers (e.g., enantiomeric isomers, diastereomeric isomers), and atropisomers. These stereoisomers can be separated, purified and enriched by asymmetric synthesis methods or chiral separation methods (including but not limited to thin-layer chromatography, rotational chromatography, column chromatography, gas chromatography, high-pressure liquid chromatography, etc.). Stereoisomers can also be obtained by bonding (chemical bonding, etc.) or salt formation (physical bonding, etc.) with other chiral compounds via chiral resolution. Optical isomers include enantiomers and diastereoisomers. All of these isomers, as well as their mixtures, are included in the scope of the present invention.

i. The term “isotopic derivative” refers to the substitution of one or more atoms in a compound by one or more atoms having a specific atomic mass or mass number. Examples of isotopes that can be incorporated into a compound include, but are not limited to, isotopes of hydrogen, carbon, nitrogen, oxygen, fluorine, sulfur, and chlorine (e.g., ²H, ³H, ¹³C, ¹⁴C, ¹⁵N, ¹⁸O, ¹⁷O, ¹⁸F, ³⁵S, and ³⁶Cl). Isotopic compounds can typically be prepared by replacing non-isotopically labeled reagents with isotopically labeled reagents according to the methods described herein. Typical examples of isotopic derivatives include deuterated compounds.

The term “pharmaceutically acceptable salt” refers to the salt of a compound prepared with a relatively non-toxic, pharmaceutically acceptable acid or base. When a compound contains a relatively acidic functional group, a base addition salt may be obtained via the exposure of the neutral form of such compound to a sufficient amount of a pharmaceutically acceptable base in a pure solution or a suitable inert solvent. The pharmaceutically acceptable base addition salts include, but are not limited to: lithium, sodium, potassium, calcium, aluminum, magnesium, zinc, bismuth, ammonium, and diethanolamine salts. When the compounds of the present invention contain relatively basic functional groups, the corresponding acid addition salts can be obtained by exposing the neutral form of such compounds to a sufficient amount of a pharmaceutically acceptable acid in a pure solution or a suitable inert solvent. Said pharmaceutically acceptable acids include inorganic acids, said inorganic acids including but not limited to: hydrochloric acid, hydrobromic acid, hydroiodic acid, nitric acid, carbonic acid, phosphoric acid, phosphorous acid, sulfuric acid, etc. Said pharmaceutically acceptable acids include organic acids, said organic acids including, but not limited to: acetic acid, propionic acid, oxalic acid, isobutyric acid, maleic acid, malonic acid, benzoic acid, succinic acid, octanedioic acid, fumaric acid, lactic acid, mandelic acid, phthalic acid, benzenesulfonic acid, p-toluenesulfonic acid, citric acid, salicylic acid, tartaric acid, methanesulfonic acid, isonicotinic acid, acid citric acid, oleic acid, tannic acid, pantothenic acid, tartaric acid hydrogen, ascorbic acid, gentianic acid, fumaric acid, gluconic acid, glycolic acid, formic acid, ethanesulfonic acid, dihydroxynaphthalic acid (i.e., 4,4′-methylene-bis(3-hydroxy-2-naphthoic acid)), amino acids (e.g., glutamic acid, arginine), etc. When the compounds contain relatively acidic and relatively basic functional groups, they can be converted into base addition salts or acid addition salts, respectively. For details, see Berge et al. “Pharmaceutical Salts”, Journal of Pharmaceutical Science 66: 1-19 (1977), or, Handbook of Pharmaceutical Salts: Properties, Selection, and Use (P. Heinrich Stahl and Camille G. Wermuth, ed., Wiley-VCH, 2002).

In some embodiments, a pharmaceutically acceptable salt of a compound with the structure shown in general formula (I) as described herein may be an acid addition salt formed by a compound of general formula (I) with a pharmaceutically acceptable acid, said acid including, but not limited to: hydrogen chloride, hydrogen bromide, sulfuric acid, carbonic acid, oxalic acid, citric acid, succinic acid, tartaric acid, phosphoric acid, lactic acid, pyruvic acid, acetic acid, maleic acid, methanesulfonic acid, benzenesulfonic acid, p-toluenesulfonic acid p-toluenesulfonic acid or ferulic acid. A pharmaceutically acceptable salt of said compound with the structure shown in general formula (I) may be prepared by reaction with an equivalent or excess amount of an acid (inorganic or organic) in a suitable solvent or solvent mixture. Said acids include, but are not limited to, hydrogen chloride, hydrogen bromide, sulfuric acid, carbonic acid, oxalic acid, citric acid, succinic acid, tartaric acid, phosphoric acid, lactic acid, pyruvic acid, acetic acid, maleic acid, methanesulfonic acid, benzenesulfonic acid, p-toluenesulfonic acid, or ferulic acid. Said solvents include, but are not limited to, methanol, ethanol, methylene chloride, acetone, ethyl acetate, toluene, or tetrahydrofuran, or a mixture of any of these acids.

The term “crystalline” is defined as a structure where the ions or molecules are arranged in a defined way, in a three-dimensional space, and in a strictly periodic manner, and have a regular pattern of periodic recurrence at a certain distance apart, because of the periodic arrangement mentioned above, there can be a variety of crystalline forms, that is, the phenomenon of polycrystalline forms, also known as polymorphism.

The term “solvate” refers to a substance formed by combining molecules with a stoichiometric or non-stoichiometric solvent. The solvent molecules in a solvate may exist in an ordered or unordered arrangement. Said solvents include, but are not limited to, water, methanol, ethanol, etc.

The term “halogen” refers to fluorine, chlorine, bromine or iodine, preferably fluorine or chlorine.

The term “amino group” refers to the —NH₂ group.

The term “hydroxyl group” refers to the —OH group.

The term “cyano group” refers to the —CN group.

The term “alkyl group” refers to a saturated straight or branched monovalent hydrocarbon group having a certain number of carbon atoms. A C₁₋₄ alkyl group refers to an alkyl group having 1-4 carbon atoms, including C₁ alkyl group, C₂ alkyl group, C₃ alkyl group, and C₄ alkyl group, such as methyl group, ethyl group, n-propyl group, isopropyl group, n-butyl group, isobutyl group, sec-butyl group, tert-butyl group butyl grouptert-butyl group. Examples of alkyl groups include, but are not limited to, methyl group, ethyl group, n-propyl group, isopropyl group, n-butylgroup, isobutyl group, sec-butyl group, tert-butyl group, and pentyl group.

The term “haloalkyl group” refers to an alkyl group substituted with one or more (e.g., 1, 2, 3 or 4)halogens (e.g., fluorine, chlorine, bromine or iodine, preferably fluorine), including but not limited to —CHF₂, —CH₂F, —CF₃, —CHF—CH₂Cl.

The term “hydroxyalkyl group” refers to an alkyl group substituted with one or more (e.g., 1, 2, 3 or 4)hydroxyl groups, including but not limited to —CH₂OH, —CHOH—CH₂OH. C₁₋₄ hydroxyalkyl groups include but are not limited to —CH₂OH, —CHOH—CH₂OH, —CH(CH₂OH)—CH₂OH.

The term “alkenyl group” refers to a straight or branched monovalent hydrocarbon group with a certain number of carbon atoms and contains at least one carbon-carbon double bond, where the carbon-carbon double bond can be located anywhere within the alkenyl group

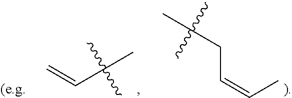

A C₂-C₆ alkenyl group refers to an alkenyl group with 2-6 carbon atoms. These include the C₂ alkenyl group, the C₃ alkenyl group, C₄ alkenyl group, C₅ alkenyl group, and C₆ alkenyl group. Examples of alkenyl groups include, but are not limited to, vinyl group, propenyl group, butenyl group, pentenyl group, hexenyl group, butadienyl group, pentadienyl group, hexadienyl group.

The term “alkoxy group” refers to —O—R^{X}, where R^{X} is an alkyl group as defined above. In some embodiments, a C₁₋₄ alkoxy group can be a methoxy group, ethoxy group, n-propoxy group, isopropoxy group, n-butoxy group, isobutoxy group, sec-butoxy group, or tert-butoxy group.

The term “cycloalkyl group” refers to a saturated monocyclic or polycyclic (e.g., concatenated, spiro, or bridged) hydrocarbon group formed by carbon atoms. In some embodiments, the cycloalkyl group is a monocyclic group. In some embodiments, a C₃₋₆ cycloalkyl group can be a cyclopropyl group, cyclobutyl group, cyclopentyl group, or cyclohexyl group. In some embodiments, a C₃₋₈ cycloalkyl group can be a C₃₋₆ cycloalkyl group, such as a cyclopropyl group, cyclobutyl group, cyclopentyl group, or cyclohexyl group.

i. The term “heteroaryl group” or “heteroaromatic ring” refers to an aromatic cyclic group formed by a carbon atom and at least one heteroatom, wherein the heteroatom may be N, O and S. A 5-membered heteroaryl group or heteroaromatic ring is, for example, a furan, thiophene, pyrrole, pyrazole, oxazole, thiazole, imidazole or triazole. 6-membered heteroaryl group or heteroaromatic ring or, for example, pyrazine, pyridazine, pyridine or pyrimidine.

The “x-y membered” in the cyclic group described in the present invitation means that the number of atoms in the ring is x-y. For example, cyclopropyl group is a 3-membered group, tetrahydropyrrolyl group is a 5-membered group, and piperidinyl group is a 6-membered group.

The term “substitution” or “substituent” refers to the substitution of one or more hydrogen atoms by a specified group. When no substitution position is specified, the substitution may be at any position, but only the formation of a stable or chemically viable chemical is permitted.

When any variable (e.g. R) appears more than once in the composition or structure of a compound, its definition in each case is independent. Thus, for example, if a group is substituted with 0-2 R's, said group may optionally be substituted with up to two R's, and there are independent options for R in each case. Furthermore, combinations of substituents and/or variables are only allowed if such combinations would yield stable compounds. For example.

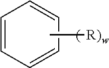

where w is 0, 1 or 2 and each R is independently a methyl group or fluorine, then

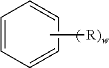

includes

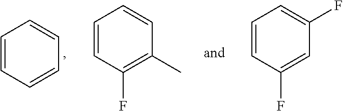

etc.

(i) The term “treatment” refers to curative therapy. In the context of a specific disease or condition, treatment means (1) alleviating one or more biological manifestations of the disease or condition, (2) interfering with (a) one or more points in the biological cascade causing or contributing to the disease or condition or (b) one or more biological manifestations of the disease or condition, (3) ameliorating one or more symptoms, effects or side effects associated with the disease or condition, or one or more symptoms, effects or side effects associated with the disease, condition or its treatment, or (4) slowing the progression of the disease, condition or one or more biological manifestations of the disease or condition.

The term “therapeutically effective amount” means an amount of a compound that, when given to a patient, is sufficient to effectively treat or prevent the disease or condition described herein. The “therapeutically effective amount” will vary depending on the compound, the disease or condition, and its severity, and the age of the patient to be treated, but may be adjusted by those skilled in the art as needed. In general, the compounds of the present invention are used in the therapeutic dose range of 1-1000 mg/day for human use. Doses beyond this range may also be used depending on the dosage form and the severity of the disease or condition.

The pharmaceutical compositions can be made into various types of dosing unit dosage forms depending on the therapeutic purpose, such as tablets, pills, powders, liquids, suspensions, emulsions, granules, pellets, capsules and injections (solutions or suspensions), preferably tablets, capsules, liquids, suspensions and injections (solutions or suspensions).

The compounds described in the present invention can be administered clinically by means of oral administration, injection, etc.

The term “subject” means any animal, preferably mammalian, preferably human, that is about to receive or has received the administration of a compound or composition. The term “mammal” includes any mammal. Examples of mammals include, but are not limited to, cattle, horses, sheep, pigs, cats, dogs, mice, rats, rabbits, guinea pigs, monkeys, humans, etc., with humans being preferred.

All patents and published publications covered herein are incorporated herein by reference in their entirety.

Each of the above preferred conditions can be combined in any way to obtain a better example of the invention without violating the common knowledge in the art.

The reagents and raw materials used in this invention are commercially available.

The positive progressive effect of the present invention is that the present invention provides a novel pyridocyclic compound that exhibits JAK inhibitory activity.

### DETAILED DESCRIPTION OF THE PREFERRED EMBODIMENT

In order to further illustrate the invention, a series of embodiments are given below, which are entirely illustrative and are used only to describe the invention specifically and should not be construed as a limitation of the invention.

### Synthesis of Key Intermediate 1

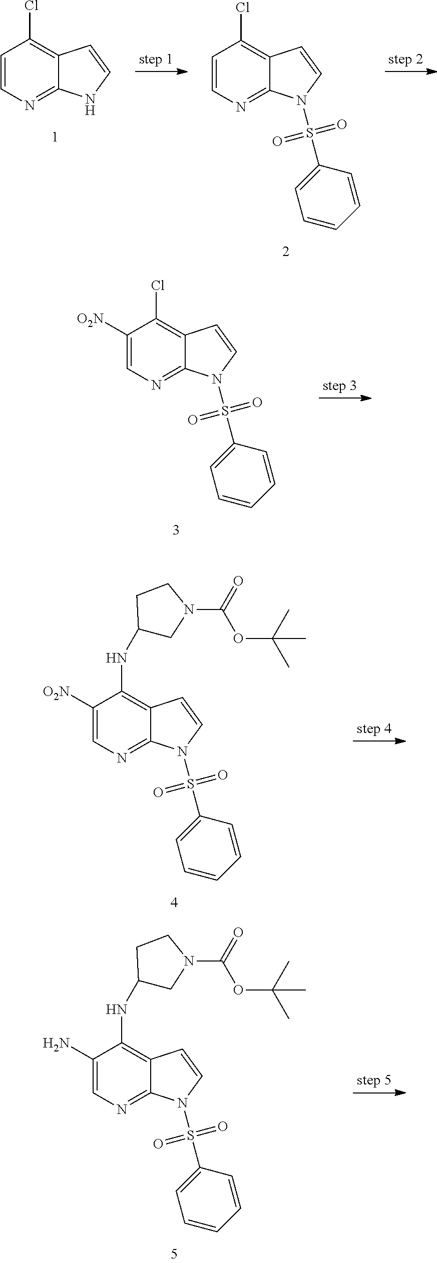 [SMILES: Clc1ccnc2[nH]ccc12 ; O=S(=O)(c1ccccc1)n1ccc2c(Cl)ccnc21 ; O=[N+]([O-])c1cnc2c(ccn2S(=O)(=O)c2ccccc2)c1Cl ; CC(C)(C)OC(=O)N1CCC(Nc2c([N+](=O)[O-])cnc3c2ccn3S(=O)(=O)c2ccccc2)C1 ; CC(C)(C)OC(=O)N1CCC(Nc2c(N)cnc3c2ccn3S(=O)(=O)c2ccccc2)C1]

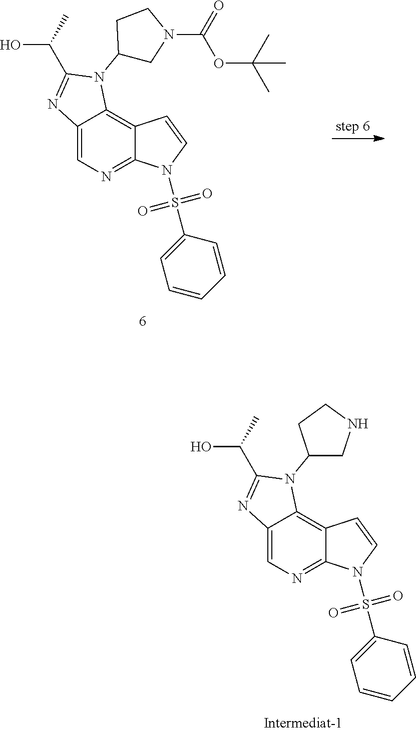 [SMILES: C[C@@H](O)c1nc2cnc3c(ccn3S(=O)(=O)c3ccccc3)c2n1C1CCN(C(=O)OC(C)(C)C)C1 ; C[C@@H](O)c1nc2cnc3c(ccn3S(=O)(=O)c3ccccc3)c2n1C1CCNC1]

Step 1: Dissolve 4-chloro-7-azaindole (25.0 g, 163.8 mmol) in 250 mL of dichloromethane, add DMAP (2.0 g, 16.5 mmol), triethylamine (34.0 mL, 245.8 mmol) and stir at room temperature for 30 min. Dissolve benzenesulfonyl chloride (23.3 mL, 180.3 mmol) in 50 mL of dichloromethane and slowly add dropwise to the above reaction solution, stir for about 4 h at room temperature and then filter, collect the filtrate and concentrate under vacuum to obtain a brown solid. 4-chloro-1-(phenylsulfonyl)-1H-pyrrolo[2,3-b]pyridine is obtained as an off-white solid (43.0 g, 90%) by triturating and curing with an appropriate amount of methanol. The product can be used directly in the next step without further purification. HRMS (ESI): m/z [M+H]⁺. C₁₃H₁₀ClNO₂₂S calculated value 293.0146, measured value 293.0139.

Step 2: Dissolve 4-chloro-1-(phenylsulfonyl)-1H-pyrrolo[2,3-b]pyridine (30.0 g, 102.7 mmol) in 300 mL of dichloromethane, add tetramethylammonium nitrate (28.0 g, 205.5 mmol) to the above solution at 25° C. and stir. Add trifluoroacetic anhydride (57.3 mL, 410.8 mmol) slowly dropwise while keeping the temperature of the reaction solution below 30° C. The mixture is stirred at room temperature for 5 h. The completion of the reaction is monitored by TLC. Saturated sodium bicarbonate is then added until the reaction solution was weakly basic, and the organic phase is separated. The aqueous phase is then extracted twice using dichloromethane. Combine the organic phases and wash with saturated NaCl solution, followed by drying over anhydrous sodium sulfate and concentrating under vacuum to yield a yellow solid. Triturating and curing with an appropriate amount of methanol yields 4-chloro-5-nitro-1-(phenylsulfonyl)-1H-pyrrolo[2,3-b]pyridine as a light yellow solid (25.9 g, 75%). The product can be used directly in the next step without further purification. ¹HNMR (300 MHz, DMSO-d₆): δ=9.09 (s, 1H), 8.29 (d, J=3.0 Hz, 1H), 8.17 (t, J=4.5 Hz, 2H), 7.79 (t, J=7.5 Hz, 1H), 7.68 (t, J=7.5 Hz, 2H), 7.11 (d, J=3.0 Hz, 1H) ppm: HRMS (ESI): m/z [M+Na]⁺. C₁₃H₈ClN₃NaO₄S calculated value 359.9816, measured value 359.9801.

Step 3: Dissolve 4-chloro-5-nitro-1-(phenylsulfonyl)-1H-pyrrolo[2,3-b]pyridine (10.0 g, 29.7 mmol) in 100 mL of tetrahydrofuran, add DIPEA (7.7 g, 59.3 mmol) and 1-Boc-3-aminopyrrolidine (8.3 g, 44.5 mmol). Stir the mixture under reflux for 4 h at an elevated temperature. The reaction was monitored for completion by TLC. The reaction is then concentrated under vacuum to yield a yellow oily liquid. Tert-butyl 3-((5-nitro-1-(phenylsulfonyl)-1H-pyrrolo[2,3-b]pyridin-4-yl)amino)pyrrolidine-1-carboxylate is obtained as a yellow solid (10.1 g, 70%) by triturating and curing with an appropriate amount of methanol. The product can be used directly in the next step without further purification. HRMS (ESI): m/z [M+H]⁺. C₂₂H₂₆N₅O₆S calculated value 488.1598, measured value 488.1607.

Step 4: Dissolve tert-butyl 3-(((5-nitro-1-(phenylsulfonyl)-1H-pyrrolo[2,3-b]pyridin-4-yl)amino)pyrrolidine-1-carboxylate (10.0 g, 20.5 mmol) in 100 mL of methanol, add palladium carbon (1 g, 10%) and keep the reaction under hydrogen atmosphere after replacing the air in the reaction flask with hydrogen gas more than three times. The reaction is carried out under hydrogen gas atmosphere at room temperature with stirring for 12 h. The completion of reaction is monitored by TLC. After extraction, the filtrate is then collected and concentrated under vacuum to yield 3-(((5-amino-1-(phenylsulfonyl)-1H-pyrrolo[2,3-b]pyridin-4-yl)amino)pyrrolidin-1-tert-butyl carboxylate as a light pink foamy solid (9.2 g, 98%). The product can be used directly in the next step without further purification. HRMS (ESI): m/z [M+H]+. C₂₂H₂₈N₅O₄S calculated value 458.1857, measured value 458.1862.

Step 5: Dissolve triethyloxonium tetrafluoroborate (11.2 g, 59.1 mmol) and (R)-lactamide (5.3 g, 59.1 mmol) in 100 mL of tetrahydrofuran, stir at room temperature for 3 h and concentrate under vacuum to obtain an oily mixture, followed by the addition of 100 mL of ethanol to dissolve and the addition of tert-butyl 3-(((5-amino-1-(phenylsulfonyl)-1H-pyrrolo[2,3-b]pyridin-4-yl)amino)pyrrolidine-1-carboxylate (9.0 g, 19.7 mmol) The reaction is then stirred while maintaining reflux for 3 h. The completion of reaction is monitored by TLC. Saturated sodium bicarbonate is then added until the reaction solution is weakly basic, and the organic phase is separated. The aqueous phase is then extracted twice using dichloromethane. Combine the organic phases and wash with saturated NaCl solution, followed by drying over anhydrous sodium sulfate and concentrating under vacuum. The residue is then purified using a silica gel column (petroleum ether:ethyl acetate=1:1) to yield tert-butyl 3-(2-((R)-1-hydroxyethyl)-6-(phenylsulfonyl)imidazo[4,5-d]pyrrolo[2,3-b]pyridin-1(6H)-yl)pyrrolidine-1-carboxylate as a light green oil (6.0 g, 60%). HRMS (ESI): m/z [M+H]⁺. C₂₅H₃₀N₅O₅S calculated value 512.1962, measured value 512.1983.

Step 6: Dissolve tert-butyl 3-(2-((R)-1-hydroxyethyl)-6-(phenylsulfonyl)imidazo[4,5-d]pyrrolo[2,3-b]pyridin-1(6H)-yl)pyrrolidine-1-carboxylate (6.0 g, 11.7 mmol) in 60 mL dichloromethane, slowly add trifluoroacetic acid (13.4 g, 117.4 mmol) and stir at room temperature for 12 h. The product is then concentrated under vacuum to yield Intermediate-1: (1R)-1-(6-(phenylsulfonyl)-1-(pyrrolidin-3-yl)-1,6-dihydroimidazo[4,5-d]pyrrolo[2,3-b]pyridin-2-yl) ethanol as a light brown oil (4.8 g, 100%). The product can be used directly in the next step without further purification. HRMS (ESI): m/z [M+H]⁺. C₂₀H₂₂N₅O₃S calculated value 412.1438, measured value 412.1448.

### Synthesis of Key Intermediate 2

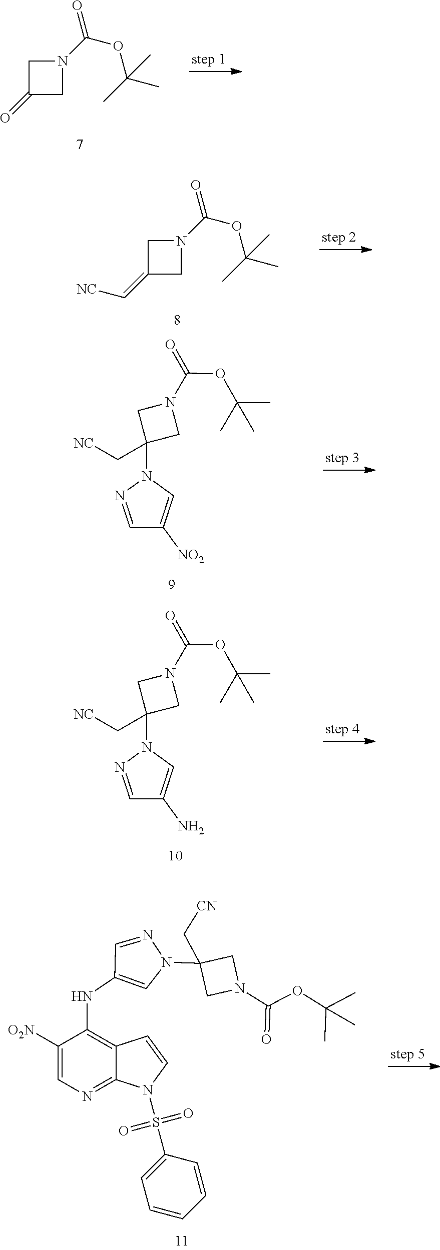 [SMILES: CC(C)(C)OC(=O)N1CC(=O)C1 ; CC(C)(C)OC(=O)N1CC(=CC#N)C1 ; CC(C)(C)OC(=O)N1CC(CC#N)(n2cc([N+](=O)[O-])cn2)C1 ; CC(C)(C)OC(=O)N1CC(CC#N)(n2cc(N)cn2)C1 ; CC(C)(C)OC(=O)N1CC(CC#N)(n2cc(Nc3c([N+](=O)[O-])cnc4c3ccn4S(=O)(=O)c3ccccc3)cn2)C1]

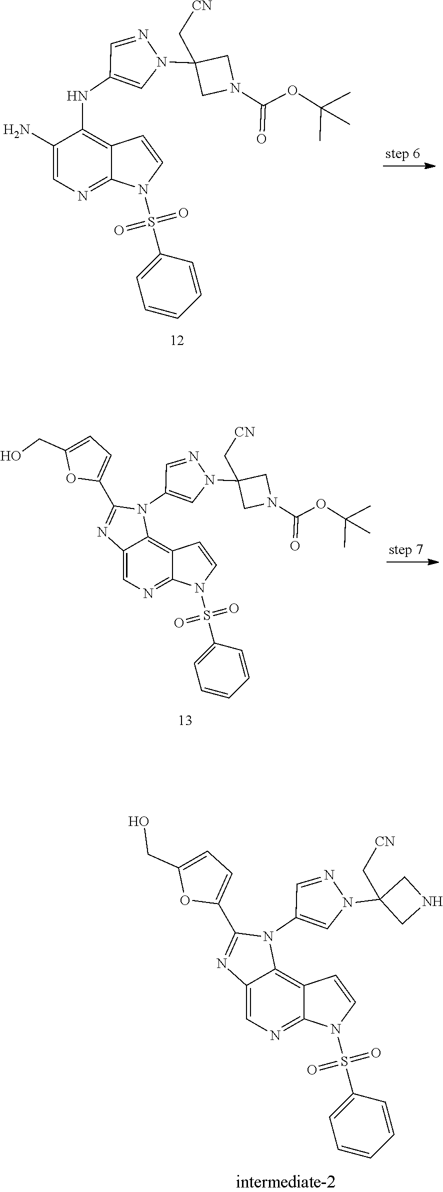 [SMILES: CC(C)(C)OC(=O)N1CC(CC#N)(n2cc(Nc3c(N)cnc4c3ccn4S(=O)(=O)c3ccccc3)cn2)C1 ; CC(C)(C)OC(=O)N1CC(CC#N)(n2cc(-n3c(-c4ccc(CO)o4)nc4cnc5c(ccn5S(=O)(=O)c5ccccc5)c43)cn2)C1 ; N#CCC1(n2cc(-n3c(-c4ccc(CO)o4)nc4cnc5c(ccn5S(=O)(=O)c5ccccc5)c43)cn2)CNC1]

Step 1: A solution of 2-diethoxyphosphoryl acetonitrile (5.7 g, 32.1 mmol) in THF (50 mL) is added to a solution of NaH (1.2 g, 30.7 mmol) in THF (50 mL) at 0° C. The mixture is then stirred at room temperature for 1 h. Then cool the mixture to 0° C., and add a solution of tert-butyl 3-oxazetidine-1-carboxylate (5.0 g, 29.2 mmol) in THF (50 mL) within 1 h. Stir the above reaction solution at room temperature for 16 hours. After the reaction is finished, add an appropriate amount of water and extract the aqueous phase twice with EA. Combine the organic layers and wash with saturated NaCl solution, dry over sodium sulfate and concentrate under vacuum to yield tert-butyl 3-(cyanomethyl)azetidine-1-carboxylate as a yellow solid (5.2 g, 78%). The product can be used directly in the next step without further purification. HRMS (EST): m/z [M+H]⁺. C₁₀H₁₅N₂O₂ calculated value 195.1128, measured value 195.1121.

Step 2: Add DBU (11.8 g, 77.4 mmol) to a solution of tert-butyl 3-(cyanomethyl)azetidine-1-carboxylate (5.0 g, 25.8 mmol) and 4-nitropyrazole (3.2 g, 28.7 mmol) in acetonitrile (30 mL). Stir the mixture at room temperature for 16 h. At the end of the reaction, add an appropriate amount of water and extract the aqueous phase twice with EA. Combine the organic layers and wash with saturated NaCl solution, dry over sodium sulfate and concentrate under vacuum. The residue is then purified using a silica gel column (petroleum ether:ethyl acetate=4:1) to yield tert-butyl 3-(cyanomethyl)-3-(4-nitro-1H-pyrazol-1-yl)azetidine-1-carboxylate as a white oil (4.0 g, 50%). HRMS (EST): m/z [M+H]⁺. C₁₃H₁₈N₅O₄ calculated value 308.1353, measured value 308.1346.

Step 3: Dissolve tert-butyl 3-(cyanomethyl)-3-(4-nitro-1H-pyrazol-1-yl)azetidine-1-carboxylate (4.0 g, 13.0 mmol) in 50 mL of methanol, add palladium carbon (0.4 g, 10%) and then use hydrogen gas to replace the air in the reaction flask more than three times. Keep the reaction under hydrogen gas atmosphere and stir for 12 h at room temperature. The completion of the reaction is monitored by TLC. The filtrate is then collected and concentrated under vacuum to yield tert-butyl 3-(4-amino-1H-pyrazol-1-yl)-3-(cyanomethyl)azetidine-1-carboxylate as a light brown foam solid (3.5 g, 98%). The product can be used directly in the next step without further purification. HRMS (ESI): m/z [M+H]⁺. C₁₃H₂₀N₅O₂ calculated value 278.1612, measured value 278.1610.

Step 4: Dissolve 4-chloro-5-nitro-1-(phenylsulfonyl)-1H-pyrrolo[2,3-b]pyridine (3.5 g, 10.5 mmol) in 100 mL of tetrahydrofuran. Add DIPEA (4.1 g, 31.5 mmol) and tert-butyl 3-(4-amino-1H-pyrazol-1-yl)-3-(cyanomethyl)azetidine-1-carboxylate (3.5 g, 12.6 mmol) and then stir under reflux at an elevated temperature for 4 h. The completion of the reaction is monitored by TLC. The reaction is then concentrated under vacuum to yield a yellow oily liquid. Tert-butyl 3-(cyanomethyl)-3-(4-((5-nitro-1-(phenylsulfonyl)-1H-pyrrolo[2,3-b]pyridin-4-yl)amino)-1H-pyrazol-1-yl)azetidine-1-carboxylate is obtained as a yellow solid (4.3 g, 70%) by triturating and curing using an appropriate amount of methanol. The product can be used directly in the next step without further purification. HRMS (ESI): m/z [M+H]⁺. C₂₆H₂₇N₅O₆S calculated value 579.1769, measured value 579.1782.

Step 5: Dissolve tert-butyl 3-(cyanomethyl)-3-(4-((5-nitro-1-(phenylsulfonyl)-1H-pyrrolo[2,3-b]pyridin-4-yl)amino)-1H-pyrazol-1-yl)azetidine-1-carboxylate ester (4.3 g, 7.4 mmol) in 50 mL of methanol, add palladium carbon (0.4 g, 10%) and replace the air in the reaction flask with hydrogen gas more than three times. The reaction is then carried out under hydrogen gas atmosphere with stirring at room temperature for 12 h. The completion of the reaction is monitored by TLC. After extraction, collect the filtrate and concentrate under vacuum to yield tert-butyl 3-(4-((5-amino-1-(phenylsulfonyl)-1H-pyrrolo[2,3-b]pyridin-4-yl)amino)-1H-pyrazol-1-yl)-3-(cyanomethyl)azetidine-1-carboxylate as a light brown foamy solid (4.0 g, 98%). The product can be used directly in the next step without further purification. HRMS (ESI): m/z [M+H]⁺. C₂₆H₂₉N₅O₄S calculated value 549.2027, measured value 549.2043.

Step 6: Dissolve tert-butyl 3-(4-((5-amino-1-(phenylsulfonyl)-1H-pyrrolo[2,3-b]pyridin-4-yl)amino)-1H-pyrazol-1-yl)-3-(cyanomethyl)azetidine-1-carboxylate (4.0 g, 7.3 mmol) in 50 mL of DMF. Then add Na₂S₂O₅ (6.9 g, 36.5 mmol) and 5-hydroxymethyl furfural (1.8 g, 14.6 mmol), and the reaction is stirred for 12 h at 90° C. The completion of the reaction is monitored by TLC. Saturated sodium bicarbonate is then added until the reaction solution is weakly basic, and the organic phase is separated. Extract the aqueous phase twice using dichloromethane. Combine the organic phases and wash with saturated NaCl solution, dry over anhydrous sodium sulfate and concentrate under vacuum. The residue is then purified using a silica gel column (petroleum ether:ethyl acetate=1:1) to yield 3-(cyanomethyl)-3-(4-(2-(5-(hydroxymethyl)furan-2-yl)-6-(phenylsulfonyl)imidazo[4,5-d]pyrrolo[2,3-b]pyridin-1(6H)-yl)-1H-pyrazol-1-yl)azetidine-1-carboxylate as a yellow solid (3.3 g, 69%). HRMS (ESI): m/z [M+H]⁺. C₃₂H₃₁N₈O₆S calculated value 655.2082, measured value 655.2063.

Step 7: Dissolve 3-(cyanomethyl)-3-(4-(2-(5-(hydroxymethyl)furan-2-yl)-6-(phenylsulfonyl)imidazo[4,5-d]pyrrolo[2,3-b]pyridin-1(6H)-yl)-1H-pyrazol-1-yl)azetidine-1-carboxylate (3.0 g, 4.6 mmol) in 60 mL of dichloromethane, slowly add trifluoroacetic acid (5.2 g, 46.0 mmol) and stir at room temperature for 12 h. Concentrate under vacuum to yield Intermediate 2: 2-(3-(4-(2-(5-(hydroxymethyl)furan-2-yl)-6-(phenylsulfonyl)imidazo[4,5-d]pyrrolo[2,3-b]pyridin-1(6H)-yl)-1H-pyrazol-1-yl)azetidin-3-yl) acetonitrile as a light yellow oil (2.3 g, 91%). The product can be used directly in the next step without further purification. HRMS (ESI): m/z [M+H]⁺. C₂₇H₂₃N₅O₄S calculated value 555.1557, measured value 555.1555.

### Synthesis of Key Intermediate 3 and Intermediate 4

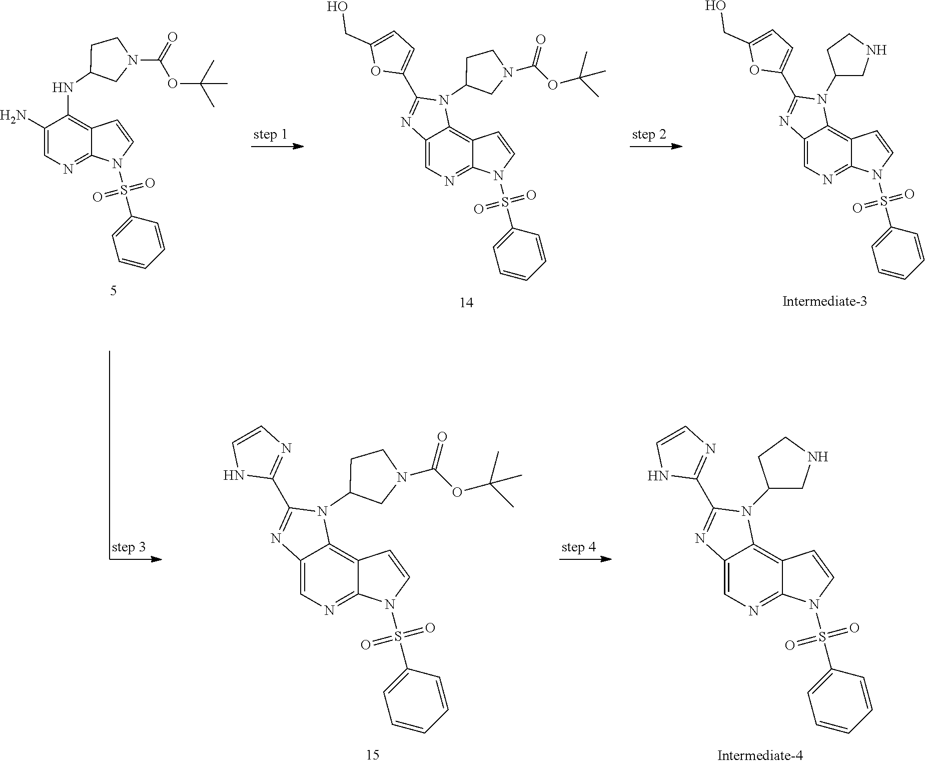 [SMILES: CC(C)(C)OC(=O)N1CCC(Nc2c(N)cnc3c2ccn3S(=O)(=O)c2ccccc2)C1 ; CC(C)(C)OC(=O)N1CCC(n2c(-c3ccc(CO)o3)nc3cnc4c(ccn4S(=O)(=O)c4ccccc4)c32)C1 ; O=S(=O)(c1ccccc1)n1ccc2c1ncc1nc(-c3ccc(CO)o3)n(C3CCNC3)c12 ; CC(C)(C)OC(=O)N1CCC(n2c(-c3ncc[nH]3)nc3cnc4c(ccn4S(=O)(=O)c4ccccc4)c32)C1 ; O=S(=O)(c1ccccc1)n1ccc2c1ncc1nc(-c3ncc[nH]3)n(C3CCNC3)c12]

Step 1: Dissolve tert-butyl 3-(((5-amino-1-(phenylsulfonyl)-1H-pyrrolo[2,3-b]pyridin-4-yl)amino)pyrrolidine-1-carboxylate (3.0 g, 6.6 mmol) in 50 mL of DMF and add Na₂S₂O₅ (6.2 g, 32.8 mmol) and 5-hydroxymethylfurfural (1.7 g, 13.2 mmol). The mixture is then stirred at 90° C. for 12 h. the completion of the reaction is monitored by TLC. Saturated sodium bicarbonate is then added until the reaction solution is weakly basic, and the organic phase is separated. Extract the aqueous phase twice with dichloromethane. Combine the organic phases and wash with saturated NaCl solution, dry over anhydrous sodium sulfate and concentrate under vacuum. The residue is then purified using a silica gel column (petroleum ether:ethyl acetate=1:1) to yield tert-butyl 3-(2-(5-(hydroxymethyl)furan-2-yl)-6-(phenylsulfonyl)imidazo[4,5-d]pyrrolo[2,3-b]pyridin-1(6H)-yl pyrrolidine-1-carboxylate as a yellow solid (2.4 g, 65%). HRMS (ESI): m/z [M+H]⁺. C₂₈H₃₀N₅O₆S calculated value 564.1911, measured value 564.1921.

Step 2: Dissolve tert-butyl 3-(2-(5-(hydroxymethyl)furan-2-yl)-6-(phenylsulfonyl)imidazo[4,5-d]pyrrolo[2,3-b]pyridin-1(6H)-yl pyrrolidine-1-carboxylate (2.0 g, 3.6 mmol) in 50 mL dichloromethane, slowly add trifluoroacetic acid (4.1 g, 36.0 mmol) at room temperature and then stir for 12 h. The product is then concentrated under vacuum to yield

Intermediate-3:(5-(6-(phenylsulfonyl)-1-(pyrrolidin-3-yl)-1,6-dihydroimidazo[4,5-d]pyrrolo[2,3-b]pyridin-2-yl)furan-2-methanol as a light yellow oil (1.5 g, 91%). The product can be used directly in the next step without further purification. HRMS (ESI): m/z [M+H]⁺. C₂₃H₂₂N₅O₄S calculated value 464.1387, measured value 464.1385.

Step 3: Dissolve tert-butyl 3-(((5-amino-1-(phenylsulfonyl)-1H-pyrrolo[2,3-b]pyridin-4-yl)amino)pyrrolidine-1-carboxylate (3.0 g, 6.6 mmol) in 50 mL of DMF and add Na₂S₂O₅ (6.2 g, 32.8 mmol) and 2-imidazolecarboxaldehyde (1.3 g, 13.2 mmol). The mixture is then stirred at 90° C. for 12 h. The completion of the reaction is monitored by TLC. Saturated sodium bicarbonate is then added until the reaction solution is weakly basic, and the organic phase is separated. Extract the aqueous phase twice using dichloromethane. Combine the organic phases and wash with saturated NaCl solution, dry over anhydrous sodium sulfate and concentrate under vacuum. The residue is then purified using a silica gel column (petroleum ether:ethyl acetate=1:1) to yield tert-butyl 3-(2-(1H-imidazol-2-yl)-6-(phenylsulfonyl)imidazo[4,5-d]pyrrolo[2,3-b]pyridin-1(6H)-yl)pyrrolidine-1-carboxylate as a yellow solid (2.2 g, 63%). HRMS (ESI): m/z [M+H]⁺. C₂₆H₂₈N₇O₄S calculated value 534.1918, measured value 534.1910.

Step 4: Dissolve tert-butyl 3-(2-(1H-imidazol-2-yl)-6-(phenylsulfonyl)imidazo[4,5-d]pyrrolo[2,3-b]pyridin-1(6H)-yl)pyrrolidine-1-carboxylate (2.0 g, 3.8 mmol) in 50 mL of dichloromethane, slowly add trifluoroacetic acid (4.3 g, 38.0 mmol) and stir at room temperature for 12 h. The product is then concentrated under vacuum to yield Intermediate 4: 2-(1H-imidazol-2-yl)-6-(phenylsulfonyl)-1-(pyrrolidin-3-yl)-1,6-dihydroimidazo[4,5-d]pyrrolo[2,3-b]pyridine as a light yellow oil (1.2 g, 74%). The product can be used directly in the next step without further purification. HRMS (ESI): m/z [M+H]⁺. C₂₁H₂₀N₇O₂S calculated value 434.1394, measured value 434.1390.

### Synthesis of Key Intermediates 5, Intermediate 6, Intermediate 7 and Intermediate 8

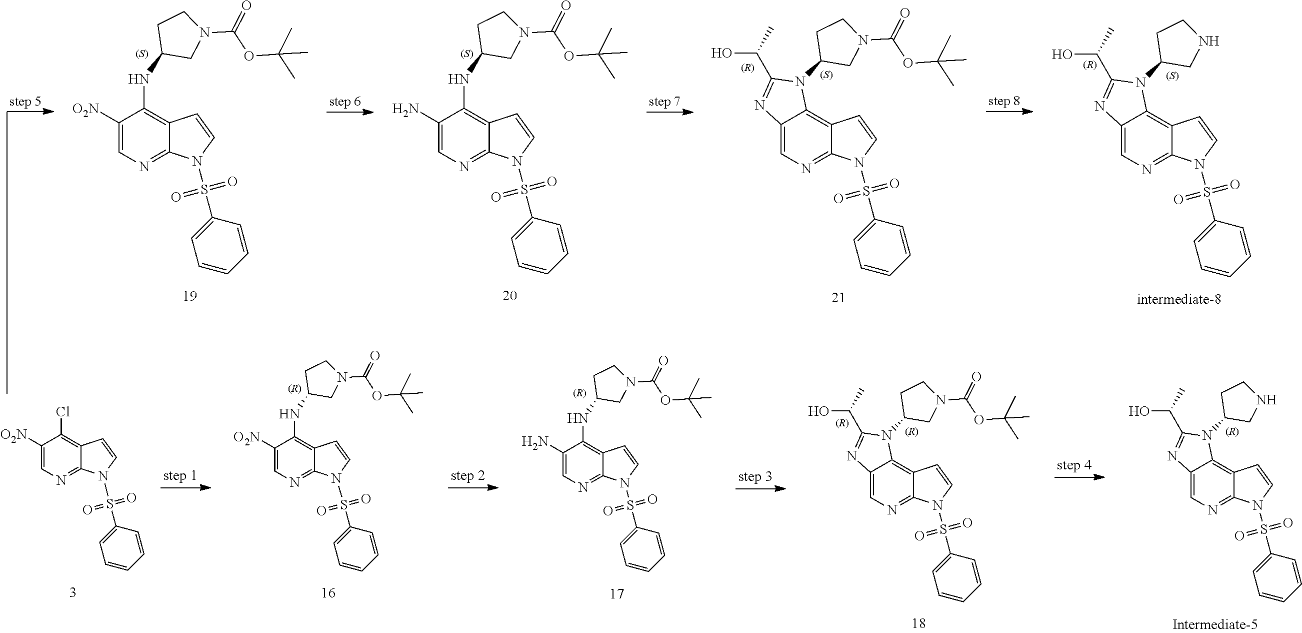

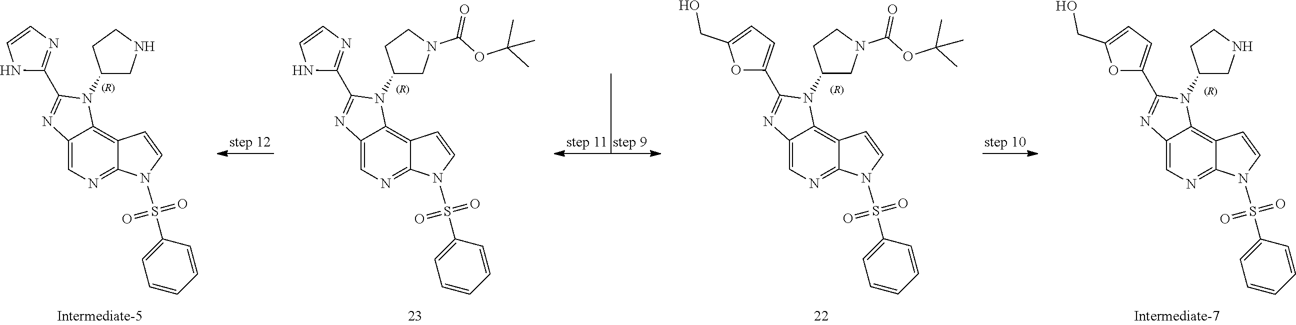

Step 1: Dissolve 4-chloro-5-nitro-1-(phenylsulfonyl)-1H-pyrrolo[2,3-b]pyridine (5.0 g, 14.8 mmol) in 50 mL of tetrahydrofuran, add DIPEA (3.8 g, 29.7 mmol) and (R)-(+)-1-Boc-3-aminopyrrolidine (4.1 g, 22.3 mmol). The mixture is then stirred while maintaining reflux at an elevated temperature for 4 hours. The completion of the reaction is monitored by TLC. Concentrate under vacuum to yield a yellow oily liquid. Triturating and curing with an appropriate amount of methanol yields tert-butyl 3-((5-nitro-1-(phenylsulfonyl)-1H-pyrrolo[2,3-b]pyridin-4-yl)amino)pyrrolidine-1-carboxylate (R) as a yellow solid (4.8 g, 66%). The product can be used directly in the next step without further purification. HRMS (ESI): m/z [M+H]⁺. C₂₂H₂₆N₅O₆S calculated value 488.1598, measured value 488.1605.

Step 2: Dissolve tert-butyl 3-((5-nitro-1-(phenylsulfonyl)-1H-pyrrolo[2,3-b]pyridin-4-yl)amino)pyrrolidine-1-carboxylate (R) (4.8 g, 9.9 mmol) in 50 mL of methanol, add palladium carbon (0.5 g, 10%) and replace the air in the reaction flask more than three times using hydrogen gas. The reaction is then carried out under hydrogen gas atmosphere with stirring at room temperature for 12 h. The completion of the reaction is monitored by TLC. The filtrate is then collected and concentrated under vacuum to yield (R) tert-butyl 3-(((5-amino-1-(phenylsulfonyl)-1H-pyrrolo[2,3-b]pyridin-4-yl)amino)pyrrolidine-1-carboxylate as a light pink foamy solid (4.2 g, 93%). The product can be used directly in the next step without further purification. HRMS (ESI): m/z. [M+H]⁺. C₂₂H₂₈N₅O₄S calculated value 458.1857, measured value 458.1850.

Step 3: Dissolve triethyloxonium tetrafluoroborate (5.0 g, 26.3 mmol) and (R)-lactamide (2.3 g, 26.3 mmol) in 80 mL of tetrahydrofuran, stir for 3 h at room temperature and then concentrate the mixture under vacuum to obtain an oil-like substance, followed by adding 80 mL of ethanol to dissolve and adding (R)tert-butyl 3-(((5-amino-1-(phenylsulfonyl)-1H-pyrrolo[2,3-b]pyridin-4-yl)amino)pyrrolidine-1-carboxylate (4.0 g, 8.8 mmol). The reaction is then stirred while maintaining reflux for 3 h and the completion of the reaction is monitored by TLC. Saturated sodium bicarbonate is then added until the reaction solution is weakly basic, and the organic phase is separated. Extract the aqueous phase twice using dichloromethane. Combine the organic phases and wash with saturated NaCl solution, dry over anhydrous sodium sulfate and concentrate under vacuum. The residue is then purified using a silica gel column (petroleum ether:ethyl acetate=1:1) to yield (R)-tert-butyl 3-(2-((R)-1-hydroxyethyl)-6-(phenylsulfonyl)imidazo[4,5-d]pyrrolo[2,3-b]pyridin-1(6H)-pyrrolidine-1-carboxylate as a light green oil (3.2 g, 72%). HRMS (ESI): m/z [M+H]⁺. C₂₅H₃₀N₅O₅S calculated value 512.1962, measured value 512.1974.

Step 4: Dissolve (R)-tert-butyl 3-(2-((R)-1-hydroxyethyl)-6-(phenylsulfonyl)imidazo[4,5-d]pyrrolo[2,3-b]pyridin-1(6H)-pyrrolidine-1-carboxylate (3.0 g, 5.9 mmol) in 50 mL of dichloromethane, slowly add trifluoroacetic acid (6.7 g, 58.6 mmol) and then stir for 12 h at room temperature. Concentrate under vacuum to yield Intermediate 5: (R)-1-(6-(phenylsulfonyl)-1-((R-pyrrolidin-3-yl)-1,6-dihydroimidazo[4,5-d]pyrrolo[2,3-b]pyridin-2-yl)ethanol as a light brown oil (2.2 g, 91%). The product can be used directly in the next step without further purification. HRMS (ESI): m/z [M+H]⁺. C₂₀H₂₂N₅O₃S calculated value 412.1438, measured value 412.1433.

Step 5: Dissolve 4-chloro-5-nitro-1-(phenylsulfonyl)-1H-pyrrolo[2,3-b]pyridine (8.0 g, 23.7 mmol) in 80 mL of tetrahydrofuran. Add DIPEA (6.1 g, 47.5 mmol) and (S)-(−)-1-Boc-3-aminopyrrolidine (6.6 g, 35.6 mmol). Then, stir the reaction while maintaining reflux at an elevated temperature for 4 hours and monitor the reaction for completion by TLC. Concentrated under vacuum to yield a yellow oily liquid. Triturate and cure with an appropriate amount of methanol to yield tert-butyl 3-((5-nitro-1-(phenylsulfonyl)-1H-pyrrolo[2,3-b]pyridin-4-yl)amino)pyrrolidine-1-carboxylate (S) as a yellow solid (8.1 g, 70%). The product can be used directly in the next step without further purification. HRMS (ESI): m/z [M+H]⁺. C₂₂H₂₆N₅O₆S calculated value 488.1598, measured value 488.1603.

Step 6: Dissolve tert-butyl 3-((5-nitro-1-(phenylsulfonyl)-1H-pyrrolo[2,3-b]pyridin-4-yl)amino)pyrrolidine-1-carboxylate (S) (8.0 g, 16.4 mmol) in 80 mL of methanol, add palladium carbon (0.8 g, 10%) and replace the air in the reaction vial more than three times using hydrogen gas. The reaction is then carried out under hydrogen gas atmosphere and stirred at room temperature for 12 h. The completion of the reaction is monitored by TLC. The filtrate is then collected and concentrated under vacuum to yield (S) tert-butyl 3-(((5-amino-1-(phenylsulfonyl)-1H-pyrrolo[2,3-b]pyridin-4-yl)amino)pyrrolidine-1-carboxylate as a light pink foamy solid (7.4 g, 99%). The product can be used directly in the next step without further purification. HRMS (ESI): m/z [M+H]⁺. C₂₂H₂₈N₅O₄S calculated value 458.1857, measured value 458.1850.

Step 7: Dissolve triethyloxonium tetrafluoroborate (8.7 g, 46.0 mmol) and (R)-lactamide (4.1 g, 46.0 mmol) in 100 mL of tetrahydrofuran, stir for 3 h at room temperature and then concentrate the mixture in vacuum to obtain an oily substance, followed by the addition of 100 mL of ethanol to dissolve, and the addition of (S) tert-butyl 3-(((5-amino-1-(phenylsulfonyl)-1H-pyrrolo[2,3-b]pyridin-4-yl)amino)pyrrolidine-1-carboxylate (7.0 g, 15.3 mmol) The mixture is then stirred while maintaining reflux at an elevated temperature for 3 h and the completion of the reaction is monitored by TLC. Saturated sodium bicarbonate is then added until the reaction solution is weakly basic, and the organic phase is separated. Extract the aqueous phase twice using dichloromethane. Combine the organic phases and wash with saturated NaCl solution, dry over anhydrous sodium sulfate and concentrate under vacuum. The residue is then purified using a silica gel column (petroleum ether:ethyl acetate =1:1) to yield (S)-tert-butyl 3-(2-((R)-1-hydroxyethyl)-6-(phenylsulfonyl)imidazo[4,5-d]pyrrolo[2,3-b]pyridin-1(6H)-pyrrolidine-1-carboxylate as a light green oil (5.3 g, 68%). HRMS (ESI): m/z [M+H]⁺. C₂₅H₃₀N₅O₅S calculated value 512.1962, measured value 512.1969.

Step 8: Dissolve (S)-tert-butyl 3-(2-((R)-1-hydroxyethyl)-6-(phenylsulfonyl)imidazo[4,5-d]pyrrolo[2,3-b]pyridin-1(6H)-pyrrolidine-1-carboxylate (5.0 g, 9.8 mmol) in 50 mL of dichloromethane, slowly add trifluoroacetic acid (11.2 g, 97.8 mmol) and then stir for 12 h at room temperature. Concentrate under vacuum to yield Intermediate 6: (R)-1-(6-(phenylsulfonyl)-1-((S-pyrrolidin-3-yl)-1,6-dihydroimidazo[4,5-d]pyrrolo[2,3-b]pyridin-2-yl)ethanol as a light brown oil (3.8 g, 94%). The product can be used directly in the next step without further purification. HRMS (ESI): m/z [M+H]⁺. C₂₀H₂₂N₅O₃S calculated value 412.1438, measured value 412.1433.

Step 9: Dissolve (R) tert-butyl 3-(((5-amino-1-(phenylsulfonyl)-1H-pyrrolo[2,3-b]pyridin-4-yl)amino)pyrrolidine-1-carboxylate (3.0 g, 6.6 mmol) in 50 mL of DMF. Then, add NaS₂O₅ (6.2 g, 32.8 mmol) and 5-hydroxymethylfurfural (1.7 g, 13.2 mmol). Stir the mixture at 90° C. for 12 h. The completion of the reaction is monitored by TLC. Saturated sodium bicarbonate is then added until the reaction solution is weakly basic, and the organic phase is separated. Extract the aqueous phase twice using dichloromethane. Combine the organic phases and wash with saturated NaCl solution, dry over anhydrous sodium sulfate and concentrate under vacuum. The residue is then purified using a silica gel column (petroleum ether:ethyl acetate=1:1) to yield (R)-tert-butyl 3-(2-(5-(hydroxymethyl)furan-2-yl)-6-(phenylsulfonyl)imidazo[4,5-d]pyrrolo[2,3-b]pyridin-1(6H)-yl)pyrrolidine-1-carboxylate as a yellow solid (2.0 g, 54%). HRMS (EST): m/z [M+H]⁺. C₂₈H₃₀N₅O₆S calculated value 564.1911, measured value 564.1922.

Step 10: Dissolve (R)-tert-butyl 3-(2-(5-(hydroxymethyl)furan-2-yl)-6-(phenylsulfonyl)imidazo[4,5-d]pyrrolo[2,3-b]pyridin-1(6H)-yl)pyrrolidine-1-carboxylate (2.0 g, 3.6 mmol) in 30 mL of dichloromethane, slowly add trifluoroacetic acid (4.1 g, 36.0 mmol) and stir for 12 h at room temperature. Intermediate 7, which is (R)-(5-(6-(phenylsulfonyl)-1-(pyrrolidin-3-yl)-1,6-dihydroimidazo[4,5-d]pyrrolo[2,3-b]pyridin-2-yl) furan-2-yl methanol is then obtained as a light yellow oil (1.6 g, 97%). The product can be used directly in the next step without further purification. HRMS (ESI): m/z [M+H]⁺. C₂₃H₂₂N₅O₄S calculated value 464.1387, measured value 464.1380.

Step 11: Dissolve (R) tert-butyl 3-(((5-amino-1-(phenylsulfonyl)-1H-pyrrolo[2,3-b]pyridin-4-yl)amino)pyrrolidine-1-carboxylate (3.0 g, 6.6 mmol) in 50 mL of DMF. Then, add Na₂S₂O₅ (6.2 g, 32.8 mmol) and 2-imidazolecarboxaldehyde (1.3 g, 13.2 mmol) and stir the mixture at 90° C. for 12 h. The completion of the reaction is monitored by TLC. Saturated sodium bicarbonate is then added until the reaction solution is weakly basic, and the organic phase is separated. Extract the aqueous phase twice using dichloromethane. Combine the organic phases and wash with saturated NaCl solution, dry over anhydrous sodium sulfate and concentrate under vacuum. The residue is then purified using a silica gel column (petroleum ether:ethyl acetate=1:1) to yield (R)-tert-butyl 3-(2-(l H-imidazol-2-yl)-6-(phenylsulfonyl)imidazo[4,5-d]pyrrolo[2,3-b]pyridin-1(6H)-pyrrolidine-1-carboxylate as a yellow solid (2.5 g, 71%). HRMS (ESI): m/z [M+H]⁺. C₂₆H₂₈N₇O₄S calculated value 534.1918, measured value 534.1910.

Step 12: Dissolve (R)-tert-butyl 3-(2-(1H-imidazol-2-yl)-6-(phenylsulfonyl)imidazo[4,5-d]pyrrolo[2,3-b]pyridin-1(6H)-pyrrolidine-1-carboxylate (2.5 g, 4.7 mmol) in 30 mL of dichloromethane, slowly add trifluoroacetic acid (5.3 g, 46.9 mmol) and then stir at room temperature for 12 h. Concentrate under vacuum to obtain Intermediate 8: (R)-2-(1H-imidazol-2-yl)-6-(phenylsulfonyl)-1-(pyrrolidin-3-yl)-1,6-dihydroimidazo[4,5-d]pyrrolo[2,3-b]pyridine as a light yellow oil (1.7 g, 84%). The product can be used directly in the next step without further purification. HRMS (ESI): m/z [M+H]⁺. C₂₁H₂₀N₇O₂S calculated value 434.1394, measured value 434.1390.

### Synthesis of Key Intermediate 9

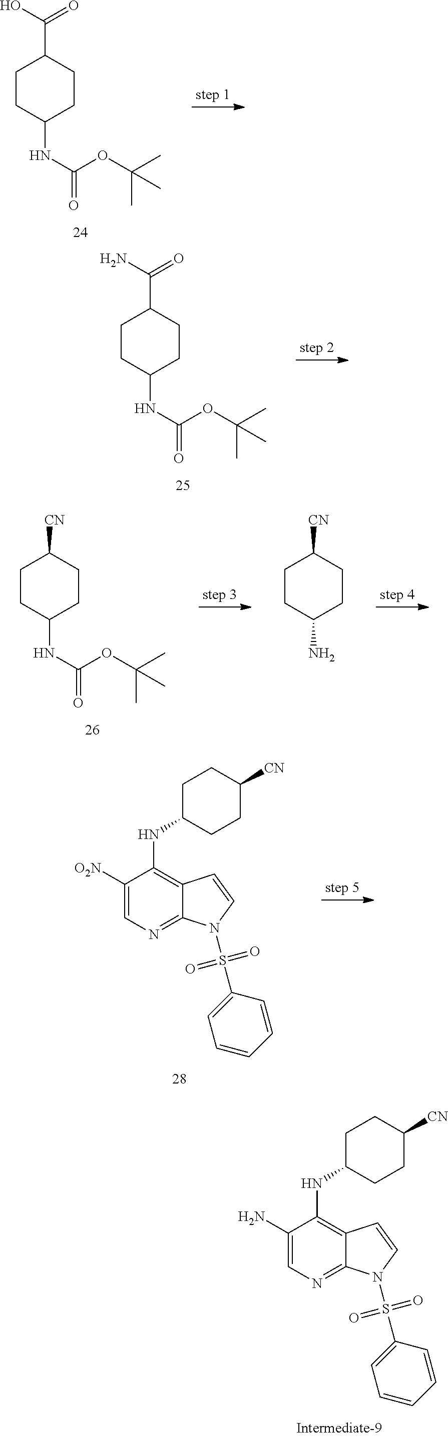

Step 1: Dissolve 4-((tert-butoxycarbonyl)amino)cyclohexanecarboxylic acid (10.0 g, 41.1 mmol) in 100 mL of tetrahydrofuran and cool down to −15° C. Add isobutyl chloroformate (11.3 g, 82.2 mmol) at low temperature and stir at low temperature for 1 h. Then, slowly add 100 mL of ammonia and stir at room temperature for 4 h. After the reaction is complete, filter the product and tert-butyl (4-carbamoylcyclohexyl)carbamate is obtained as a white solid (8.0 g, 80%). The product can be used directly in the next step without further purification. HRMS (ESI): m/z [M+H]⁺. C₁₂H₂₃N₂O₃ calculated value 243.1703, measured value 243.1698.

Step 2: Dissolve tert-butyl (4-carbamoylcyclohexyl)carbamate (8.0 g, 33.1 mmol) in 50 mL of pyridine and cool down to −15° C. Add phosphorus oxychloride (10 g, 66.2 mmol) slowly dropwise at low temperature and stir at low temperature for 3 h. After the reaction is complete, pour the reaction solution slowly into 200 mL of ice-water mixture. Extract the aqueous phase three times with EA. Combine the organic phases and wash with saturated NaCl solution, dry with anhydrous sodium sulfate and concentrate under vacuum to yield tert-butyl (4-cyanocyclohexyl)carbamate as a milky-white solid (5.5 g, 74%). HRMS (ESI): m/z [M+H]⁺. C₁₂H₂₁N₂O₂ calculated value 225.1598, measured value 225.1603.

Step 3: Dissolve tert-butyl (4-cyanocyclohexyl)carbamate (5.5 g, 24.6 mmol) in 50 mL of dichloromethane and slowly add trifluoroacetic acid (28.0 g, 245.5 mmol). Stir the mixture at room temperature for 12 h. The product is then concentrated under vacuum to yield trans-4-aminocyclohexanecarbonitrile as a light white solid (2.5 g, 82%). The product can be used directly in the next step without further purification. HRMS (ESI): m/z [M+H]⁺. C₇H₁₃N₂ calculated value 125.1073, measured value 125.1082.

Step 4: Dissolve 4-chloro-5-nitro-1-(phenylsulfonyl)-1H-pyrrolo[2,3-b]pyridine (4.5 g, 13.4 mmol) in 50 mL of tetrahydrofuran, add DIPEA (3.5 g, 26.9 mmol) and trans-4-aminocyclohexanecarbonitrile (2.5 g, 20.2 mmol) and stir for 4 h at an elevated temperature while maintaining reflux. The completion of the reaction is monitored by TLC. Concentrate under vacuum to obtain a yellow oily liquid. Triturate and cure with an appropriate amount of methanol to yield trans-4-((5-nitro-1-(phenylsulfonyl)-1H-pyrrolo[2,3-b]pyridin-4-yl)amino)cyclohexanecarbonitrile as a yellow solid (4.2 g, 74%). The product can be used directly in the next step without further purification. HRMS (ESI): m/z [M+H]⁺. C₂₀H₂₀N₅O₄S calculated value 426.1231, measured value 426.1237.

Step 5: Dissolve trans-4-((5-nitro-1-(phenylsulfonyl)-1H-pyrrolo[2,3-b]pyridin-4-yl)amino)cyclohexanecarbonitrile (4.2 g, 9.9 mmol) in 50 mL of methanol, add palladium carbon (0.4 g, 10%) and replace the air in the reaction flask with hydrogen gas more than three times. The reaction is then carried out under hydrogen gas atmosphere and stirred at room temperature for 12 h. The completion of the reaction is monitored by TLC. After filtration, collect the filtrate and concentrate under vacuum to yield Intermediate 9: trans-4-((5-amino-1-(phenylsulfonyl)-1H-pyrrolo[2,3-b]pyridin-4-yl)amino)cyclohexanecarbonitrile as a light brown foamy solid (3.5 g, 90%). The product can be used directly in the next step without further purification. HRMS (ESI): m/z [M+H]⁺. C₂₀H₂₂N₅O₂S calculated value 396.1489, measured value 396.1492.

### Synthesis of Key Intermediate 10 and Intermediate 11

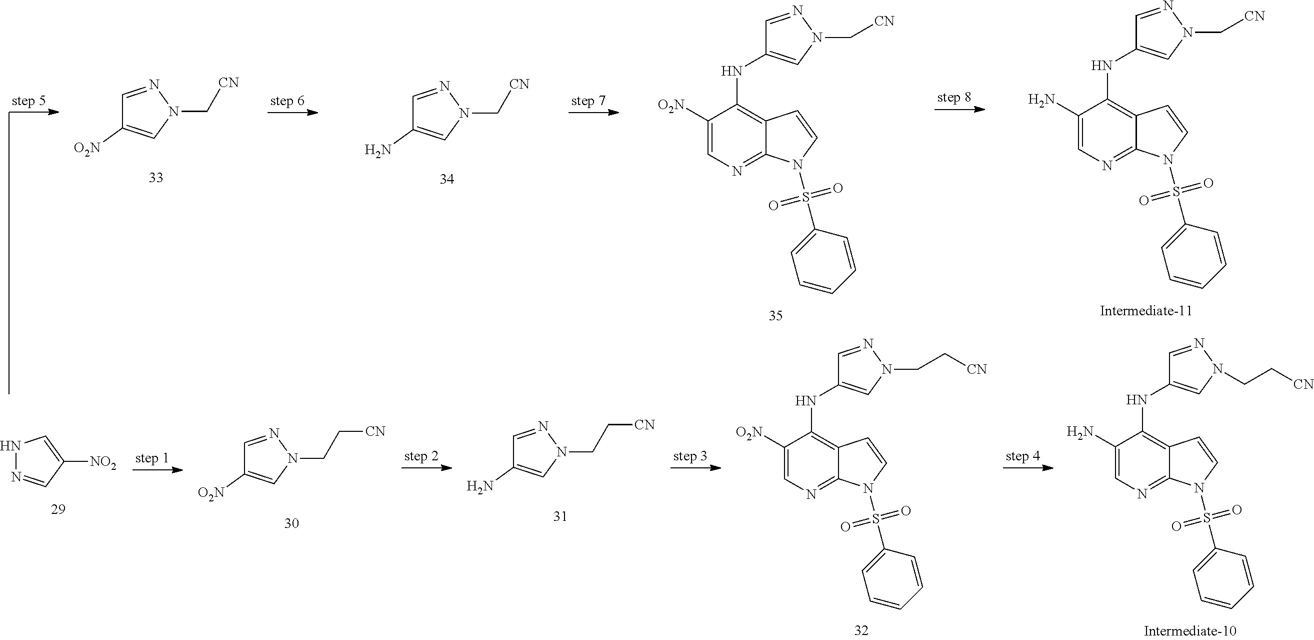

Step 1: Dissolve 4-nitropyrazole (5 g, 44.2 mmol) in 50 mL of DMF, add potassium carbonate (12.2 g, 88.4 mmol) and 3-bromopropionitrile (8.9 g, 66.3 mmol), Then, stir the mixture at room temperature for 12 h. The completion of the reaction is monitored by TLC. Saturated sodium bicarbonate is then added until the reaction solution is weakly basic, and the organic phase is separated. Extract the aqueous phase three times using dichloromethane. Combine the organic phases and wash with saturated NaCl solution, dry over anhydrous sodium sulfate and concentrate under vacuum to yield 3-(4-nitro-1H-pyrazol-1-yl)propanenitrile as a pale yellow solid (4.8 g, 65%) The product can be used directly in the next step without further purification. HRMS (ESI): m/z [M+H]⁺. C₆H₇N₄O₂ calculated value 167.0564, measured value 167.0569.

Step 2. Dissolve 3-(4-nitro-1H-pyrazol-1-yl)propanenitrile (4.8 g, 28.9 mmol) in 50 mL of methanol, add palladium carbon (0.5 g, 10%) and use hydrogen gas to replace the air in the reaction flask more than three times The reaction is then carried out under hydrogen atmosphere and stirred at room temperature for 12 h. The completion of the reaction is monitored by TLC. After filtration, the filtrate is then collected and concentrated under vacuum to yield 3-(4-amino-1H-pyrazol-1-yl)propanenitrile as a light brown foamy solid (3.5 g, 89%). The product can be used directly in the next step without further purification. HRMS (ESI): m/z [M+H]⁺. C₆H₉N₄ calculated value 137.0822, measured value 137.0833.

Step 3: Dissolve 4-chloro-5-nitro-1-(phenylsulfonyl)-1H-pyrrolo[2,3-b]pyridine (5.8 g, 17.2 mmol) in 60 mL of tetrahydrofuran. Add DIPEA (4.4 g, 34.4 mmol) and 3-(4-amino-1H-pyrazol-1-yl)propanenitrile (3.5 g, 25.7 mmol). The mixture is stirred while maintaining reflux for 4 hours at an elevated temperature and the completion of the reaction is monitored by TLC. Subsequent concentration under vacuum yields a yellow oily liquid. Triturating and curing with an appropriate amount of methanol yields 3-(4-((5-nitro-1-(phenylsulfonyl)-1H-pyrrolo[2,3-b]pyridin-4-yl)amino)-1H-pyrazol-1-yl)propanenitrile as a yellow solid (5.1 g, 68%). The product can be used directly in the next step without further purification. HRMS (ESI): m/z [M+H]⁺. C₁₉H₁₆N₇O₄S calculated value 438.0979, measured value 438.0985.

Step 4: Dissolve 3-(4-((5-nitro-1-(phenylsulfonyl)-1H-pyrrolo[2,3-b]pyridin-4-yl)amino)-1H-pyrazol-1-yl)propanenitrile (5.0 g, 11.4 mmol) in 50 mL of methanol, add palladium carbon (0.5 g, 10%) and replace the air in the reaction flask more than three times using hydrogen gas. The reaction is then carried out under hydrogen gas atmosphere and stirred at room temperature for 12 h. The completion of the reaction is monitored by TLC. After filtration, collect the filtrate and concentrate under vacuum to yield Intermediate 10: 3-(4-((5-amino-1-(phenylsulfonyl)-1H-pyrrolo[2,3-b]pyridin-4-yl)amino)-1H-pyrazol-1-yl)propanenitrile as a light yellow foamy solid (4.5 g, 97%). The product can be used directly in the next step without further purification. HRMS (ESI): m/z [M+H]⁺. C₁₉H₁₈N₇O₂S calculated value 408.1237, measured value 408.1241.

Step 5: Dissolve 4-nitropyrazole (2.5 g, 22.1 mmol) in 50 mL of DMF, add potassium carbonate (6.1 g, 44.2 mmol) and 2-bromoacetonitrile (4.0 g, 33.2 mmol) and stir at room temperature for 12 h. The completion of the reaction is monitored by TLC. Saturated sodium bicarbonate is then added until the reaction solution is weakly basic, and the organic phase is separated. Extract the aqueous phase three times using dichloromethane. Combine the organic phases and wash with saturated NaCl solution, dry over anhydrous sodium sulfate and concentrate under vacuum to yield 3-(4-nitro-1H-pyrazol-1-yl)acetonitrile as a pale yellow solid (2.2 g, 65%) The product can be used directly in the next step without further purification. HRMS (ESI): m/z [M+H]⁺. C₅H₅N₄O₂ calculated value 153.0407, measured value 153.0411.

Step 6: Dissolve 3-(4-nitro-1H-pyrazol-1-yl)acetonitrile (2.2 g, 14.5 mmol) in 30 mL of methanol, add palladium carbon (0.3 g, 10%) and then use hydrogen gas to replace the air in the reaction flask more than three times. The reaction is then carried out under hydrogen gas atmosphere and stirred at room temperature for 12 h. The completion of the reaction is monitored by TLC. After filtration, collect the filtrate and concentrate under vacuum to yield 3-(4-amino-1H-pyrazol-1-yl)acetonitrile as a light brown foamy solid (1.6 g, 91%). The product can be used directly in the next step without further purification. HRMS (ESI): m/z [M+H]⁺. C₅H₇N₄ calculated value 123.0665, measured value 123.0671.

Step 7: Dissolve 4-chloro-5-nitro-1-(phenylsulfonyl)-1H-pyrrolo[2,3-b]pyridine (2.8 g, 8.2 mmol) in 60 mL of tetrahydrofuran. Add DIPEA (2.1 g, 16.4 mmol) and 3-(4-amino-1H-pyrazol-1-yl)acetonitrile (1.5 g, 12.3 mmol) and then stir the mixture while maintaining reflux at an elevated temperature for 4 h. The completion of the reaction is monitored by TLC. Concentrate under vacuum to yield a yellow oily liquid. Then triturate and cure with an appropriate amount of methanol to yield 3-(4-((5-nitro-1-(phenylsulfonyl)-1H-pyrrolo[2,3-b]pyridin-4-yl)amino)-1H-pyrazol-1-yl)acetonitrile as a yellow solid (2.5 g, 72%). The product can be used directly in the next step without further purification. HRMS (EST): m/z [M+H]⁺. C₁₈H₁₄N₇O₄S calculated value 424.0822, measured value 424.0833.

Step 8: Dissolve 3-(4-((5-nitro-1-(phenylsulfonyl)-1H-pyrrolo[2,3-b]pyridin-4-yl)amino)-1H-pyrazol-1-yl)acetonitrile (2.5 g, 5.9 mmol) in 30 mL of methanol, add palladium carbon (0.3 g, 10%) and replace the air in the reaction flask more than three times using hydrogen gas. The reaction is then carried out under hydrogen gas atmosphere at room temperature with stirring for 12 h. The completion of the reaction is monitored by TLC. Collect the filtrate and concentrate under vacuum to yield Intermediate 11: 3-(4-((5-amino-1-(phenylsulfonyl)-1H-pyrrolo[2,3-b]pyridin-4-yl)amino)-1H-pyrazol-1-yl)acetonitrile as alight yellow foamy solid (2.0 g, 86%). The product can be used directly in the next step without further purification. HRMS (EST): m/z [M+H]⁺. C₁₈H₁₆N₇O₂S calculated value 394.1081, measured value 394.1084.

### Synthesis of Key Intermediate 12

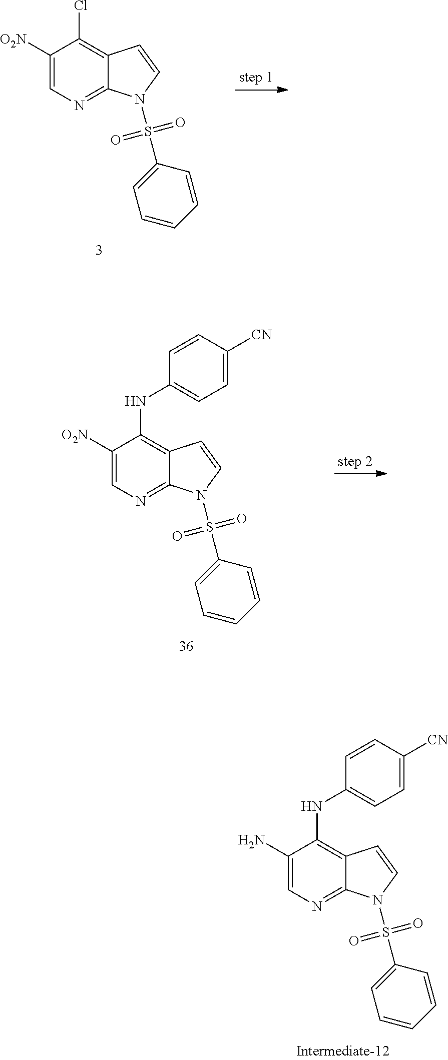 [SMILES: O=[N+]([O-])c1cnc2c(ccn2S(=O)(=O)c2ccccc2)c1Cl ; N#Cc1ccc(Nc2c([N+](=O)[O-])cnc3c2ccn3S(=O)(=O)c2ccccc2)cc1 ; N#Cc1ccc(Nc2c(N)cnc3c2ccn3S(=O)(=O)c2ccccc2)cc1]

Step 1: Dissolve 4-chloro-5-nitro-1-(phenylsulfonyl)-1H-pyrrolo[2,3-b]pyridine (2.0 g, 5.9 mmol) in 50 mL of toluene. Then, add tris(dibenzalacetone)dipalladium (0.2 g, 10%), 4,5-bis(diphenylphosphino)-9,9-dimethylxanthene (0.2 g, 10%) and p-cyanoaniline (1.4 g, 11.9 mmol). The reaction is then carried out under nitrogen gas protection and stirred while maintaining reflux at an elevated temperature for 5 h. The completion of the reaction is monitored by TLC. Saturated sodium bicarbonate is then added until the reaction solution is weakly basic, and the organic phase is separated. Extract the aqueous phase three times using dichloromethane. Combine the organic phases and wash with saturated NaCl solution, dry over anhydrous sodium sulfate and concentrate under vacuum. The residue is then purified using a silica gel column (petroleum ether:ethyl acetate=2:1) to yield 4-((5-nitro-1-(phenylsulfonyl)-1H-pyrrolo[2,3-b]pyridin-4-yl)amino)benzonitrile as a yellow solid (1.2 g, 48%). hRMS (ESI): m/z [M+H]⁺. C₂₀H₁₄N₅O₄S calculated value 420.0761, measured value 420.0771.

Step 2: Dissolve 4-((5-nitro-1-(phenylsulfonyl)-1H-pyrrolo[2,3-b]pyridin-4-yl)amino)benzonitrile (1.2 g, 2.9 mmol) in 30 mL of methanol, add palladium carbon (0.2 g, 10%) and then use hydrogen gas to replace the air in the reaction flask more than three times. The reaction is then carried out under hydrogen gas atmosphere and stirred at room temperature for 12 h. The completion of the reaction is monitored by TLC. Collect the filtrate and concentrate under vacuum to yield Intermediate 12: 4-((5-amino-1-(phenylsulfonyl)-1H-pyrrolo[2.3-b]pyridin-4-yl)amino)benzonitrile as a yellow foamy solid (1.1 g, 99%). The product can be used directly in the next step without further purification. HRMS (ESI): m/z [M+H]⁺. C₂₀H₁₆N₅O₂S calculated value 390.1019, measured value 390.1023.

### Synthesis of Key Intermediate 13

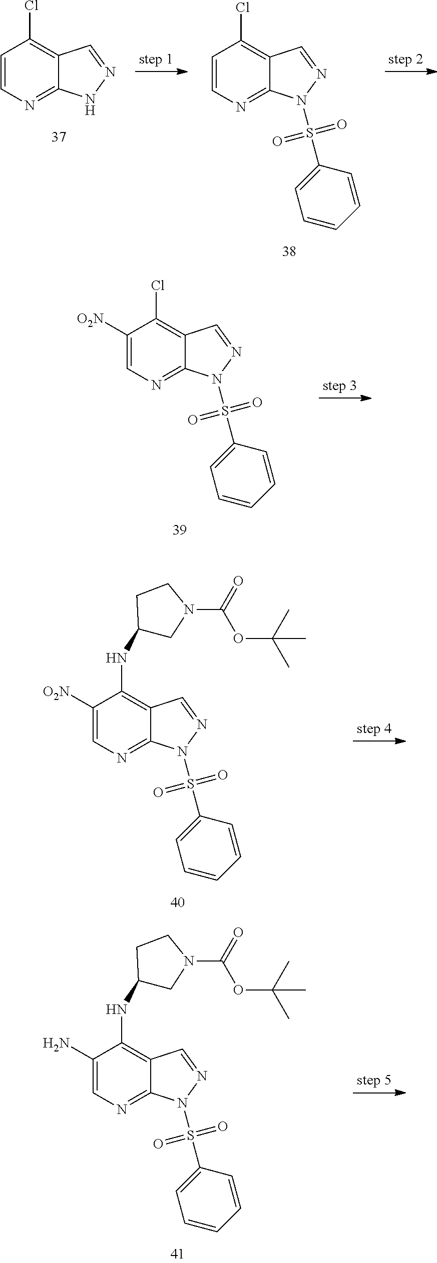 [SMILES: Clc1ccnc2[nH]ncc12 ; O=S(=O)(c1ccccc1)n1ncc2c(Cl)ccnc21 ; O=[N+]([O-])c1cnc2c(cnn2S(=O)(=O)c2ccccc2)c1Cl ; CC(C)(C)OC(=O)N1CC[C@H](Nc2c([N+](=O)[O-])cnc3c2cnn3S(=O)(=O)c2ccccc2)C1 ; CC(C)(C)OC(=O)N1CC[C@H](Nc2c(N)cnc3c2cnn3S(=O)(=O)c2ccccc2)C1]

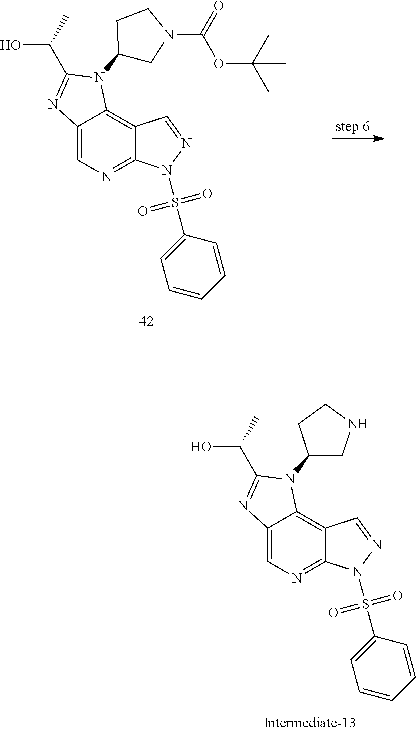 [SMILES: C[C@@H](O)c1nc2cnc3c(cnn3S(=O)(=O)c3ccccc3)c2n1[C@H]1CCN(C(=O)OC(C)(C)C)C1 ; C[C@@H](O)c1nc2cnc3c(cnn3S(=O)(=O)c3ccccc3)c2n1[C@H]1CCNC1]

Step 1; Dissolve 4-chloro-1H-pyrazolo[3,4-b]pyridine (10.0 g, 65.4 mmol) in 100 mL of dichloromethane. Add DMAP (0.8 g, 6.5 mmol), triethylamine (18.0 mL, 130.7 mmol) and stir at room temperature for 30 min. Dissolve benzenesulfonyl chloride (10.1 mL, 78.5 mmol) in 50 mL of dichloromethane and slowly add the solution dropwise to the above reaction solution, stir for about 4 h at room temperature and then filter, collect the filtrate and concentrate under vacuum to obtain a brown solid. 4-chloro-1-(phenylsulfonyl)-1H-pyrazolo[3,4-b]pyridine is then obtained as an off-white solid (17.0 g, 89%) by triturating and curing with an appropriate amount of methanol. The product can be used directly in the next step without further purification. HRMS (EST): m/z [M+H]⁺. C₁₂H₉ClN₃O₂S calculated value 294.0099, measured value 293.0089.

Step 2: Dissolve 4-chloro-1-(phenylsulfonyl)-1H-pyrazolo[3,4-b]pyridine (15.0 g, 51.2 mmol) in 150 mL of dichloromethane, add tetramethylammonium nitrate (13.9 g, 102.3 mmol) to the above solution at 25° C. and stir. Trifluoroacetic anhydride (28.6 mL, 204.8 mmol) is then added slowly dropwise while maintaining the temperature of the reaction solution below 30° C. Stir the mixture at room temperature for 5 h. The completion of the reaction is monitored by TLC. Saturated sodium bicarbonate is then added until the reaction solution is weakly basic, and the organic phase is separated. Extract the aqueous phase twice using dichloromethane. Combine the organic phases and wash with saturated NaCl solution, dry over anhydrous sodium sulfate and concentrate under vacuum to yield a yellow solid. Triturate and cure with an appropriate amount of methanol to yield 4-chloro-5-nitro-1-(phenylsulfonyl)-1H-pyrazolo[2,3-b]pyridine as alight yellow solid (12.3 g, 71%). The product can be used directly in the next step without further purification. HRMS (ESI): m/z [M+H]⁺. C₁₂H₈ClN₄O₄S calculated value 338.9949, measured value 338.9930.

Step 3: Dissolve 4-chloro-5-nitro-1-(phenylsulfonyl)-1H-pyrazolo[2,3-b]pyridine (5.0 g, 14.8 mmol) in 50 mL of tetrahydrofuran, followed by the addition of DIPEA (3.8 g, 29.6 mmol) and (S)-(−)-1-Boc-3-aminopyrrolidine (4.2 g, 22.2 mmol). The mixture is then stirred while maintaining reflux at an elevated temperature for 4 hours and the completion of the reaction is monitored by TLC. Concentrate under vacuum to yield a yellow oily liquid. Triturating and curing with an appropriate amount of methanol yields (S)tert-butyl 3-(((5-nitro-1-(phenylsulfonyl)-1H-pyrazolo[3,4-b]pyridin-4-yl)amino)pyrrolidine-1-carboxylate as a yellow solid (5.1 g, 71%). The product can be used directly in the next step without further purification. HRMS (ESI): m/z [M+H]⁺. C₂₁H₂₅N₆O₆S calculated value 489.1551, measured value 488.1559.

Step 4: Dissolve (S)tert-butyl 3-(((5-nitro-1-(phenylsulfonyl)-1H-pyrazolo[3,4-b]pyridin-4-yl)amino)pyrrolidine-1-carboxylate (5.0 g, 10.2 mmol) in 50 mL of methanol, add palladium carbon (0.5 g, 10%) and replace the air in the reaction flask with hydrogen gas more than three times. The reaction is then carried out under hydrogen gas atmosphere at room temperature with stirring for 12 h. The completion of the reaction is monitored by TLC. The filtrate is then collected and concentrated under vacuum to yield (S)tert-butyl 3-(((5-amino-1-(phenylsulfonyl)-1H-pyrazolo[3,4-b]pyridin-4-yl)amino)pyrrolidine-1-carboxylate as a pink foamy solid (4.5 g, 96%). The product can be used directly in the next step without further purification. HRMS (ESI): m/z [M+H]⁺. C₂₁H₂₇N₆O₄S calculated value 459.1809, measured value 459.1813.

Step 5: Dissolve triethyloxonium tetrafluoroborate (5.0 g, 26.1 mmol) and (R)-lactamide (2.3 g, 26.1 mmol) in 50 mL of tetrahydrofuran, stir at room temperature for 3 h and then concentrate the mixture under vacuum to obtain an oily mixture, followed by the addition of 50 mL of ethanol to dissolve and the addition of (S)tert-butyl 3-(((5-amino-1-(phenylsulfonyl)-1H-pyrazolo[3,4-b]pyridin-4-yl)amino)pyrrolidine-1-carboxylate (4.0 g, 8.7 mmol). The reaction is then stirred while maintaining reflux for 3 h at an elevated temperature. The completion of the reaction is monitored by TLC. Saturated sodium bicarbonate is then added until the reaction solution is weakly basic, and the organic phase is separated. Extract the aqueous phase twice using dichloromethane. Combine the organic phases and wash with saturated NaCl solution, dry over anhydrous sodium sulfate and concentrate under vacuum. The residue is then purified using a silica gel column (petroleum ether:ethyl acetate=1:1) to yield (S)-tert-butyl 3-(2-((R)-1-hydroxyethyl)-6-(phenylsulfonyl)imidazo[4,5-d]pyrazolo[3,4-b]pyridin-1(6H)-pyrrolidine-1-carboxylate as a green oil (2.2 g, 49%). HRMS (ESI): m/z [M+H]⁺. C₂₄H₂₉N₆O₅S calculated value 513.1915, measured value 513.1923.

Step 6: Dissolve (S)-tert-butyl 3-(2-((R)-1-hydroxyethyl)-6-(phenylsulfonyl)imidazo[4,5-d]pyrazolo[3,4-b]pyridin-1(6H)-pyrrolidine-1-carboxylate (2.0 g, 3.9 mmol) in 20 mL of dichloromethane, slowly add trifluoroacetic acid (4.4 g, 39.0 mmol) and then stir for 12 h at room temperature. Concentrate under vacuum to yield Intermediate 13: (R)-1-(6-(phenylsulfonyl)-1-((S)-pyrrolidin-3-yl)-1,6-dihydroimidazo[4,5-d]pyrazolo[3,4-b]pyridin-2-yl) ethanol as a light brown oil (1.5 g, 91%). The product can be used directly in the next step without further purification. HRMS (ESI): m/z [M+H]⁺. C₁₉H₂₁N₆O₃S calculated value 413.1390, measured value 413.1399.

### Example 1

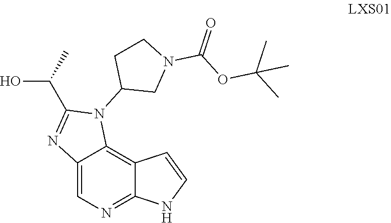 [SMILES: C[C@@H](O)c1nc2cnc3[nH]ccc3c2n1C1CCN(C(=O)OC(C)(C)C)C1]

Tert-butyl 3-(2-((R)-1-hydroxyethyl)imidazo[4,5-d]pyrrolo[2,3-b]pyridin-1(6H)-yl)pyrrolidine-1-carboxylate

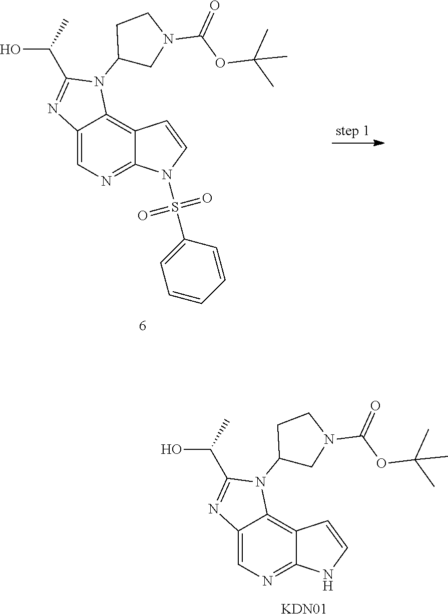

Step 1: Tert-butyl 3-(2-((R)-1-hydroxyethyl)-6-(phenylsulfonyl)imidazo[4,5-d]pyrrolo[2,3-b]pyridin-1(6H)-yl)pyrrolidine-1-carboxylate (0.4 g, 0.8 mmol) was dissolved in a solvent mixture of 5 mL tetrahydrofuran and 5 mL methanol, and stirred at room temperature for 5 h after adding 5 mL of 1 M sodium hydroxide. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate=1:1) to yield LXS01: tert-butyl 3-(2-((R)-1-hydroxyethyl)imidazo[4,5-d]pyrrolo[2,3-b]pyridin-1(6H)-yl)pyrrolidine-1-carboxylate (0.2 g, 69%), 69%). ¹ HNMR (300 MHz, DMSO-d₆): δ=11.95 (s, 1H), 8.59 (s, 1H), 7.51 (s, 1H), 6.35 (s, 1H), 5.75 (t, J=3.0 Hz, 2H), 5.17-5.21 (m, 1H), 3.82-3.93 (m, 2H), 3.76 (t, J=9.0 Hz, 1H), 3.40-3.47 (m, 1H), 2.58-2.74 (m, 1H), 2.28-2.41 (m, 1H), 1.65 (d, J 9.0 Hz, 3H), 1.45 (d, J=24.0 Hz, 9H) ppm; ¹³ C NMR (75 MHz, DMSO-d₆) δ 153.23, 148.61, 148.53, 142.1, 129.04, 127.15, 120.71, 115.63, 99.37, 79.85, 63.60, 58.31, 53.58, 48.13, 28.47, 27.05, 22.81 ppm: HRMS (ESI): m/z [M+H]⁺. C₁₉H₂₆N₅O₃ calculated value 372.2030, measured value 372.2018.

### Example 2

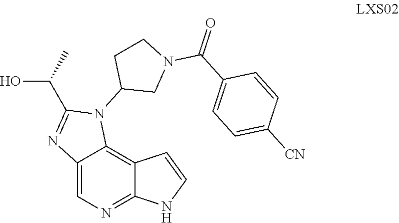 [SMILES: C[C@@H](O)c1nc2cnc3[nH]ccc3c2n1C1CCN(C(=O)c2ccc(C#N)cc2)C1]

### 4-(3-(2-((R)-1-Hydroxyethyl)imidazo[4,5-d]pyrrolo[2,3-b]pyridin-1(6H)-yl)pyrrolidine-1-carbonyl)benzonitrile

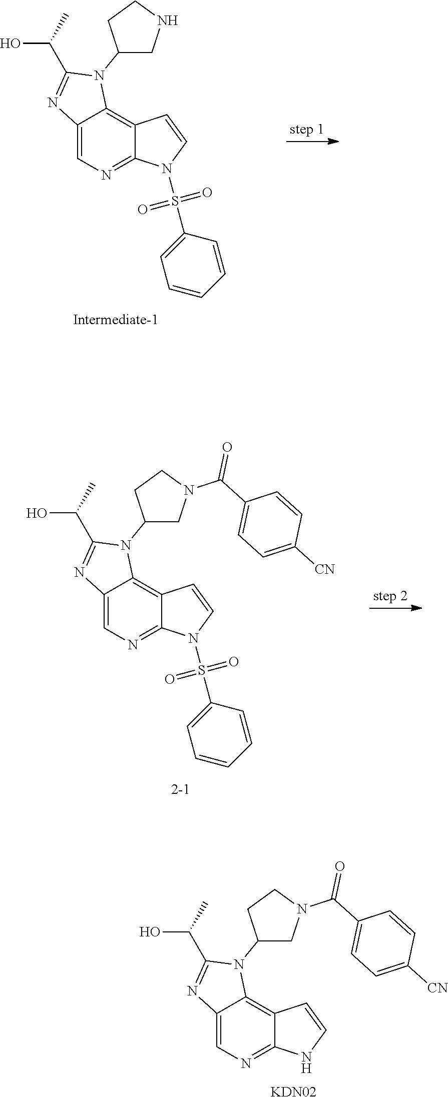

Step 1: Dissolve (1R)-1-(6-(phenylsulfonyl)-1-(pyrrolidin-3-yl)-1,6-dihydroimidazo[4,5-d]pyrrolo[2,3-b]pyridin-2-yl)ethanol (0.3 g, 0.7 mmol) in 10 mL of tetrahydrofuran, add DIPEA (0.2 g, 1.4 mmol) and then slowly add p-cyanobenzoyl chloride (0.2 g, 1.4 mmol) dropwise. Raise the temperature and stir under reflux for 3 h. Monitor the reaction for completion by TLC. Saturated sodium bicarbonate was added until the reaction solution was weakly basic, and the organic phase was separated. The aqueous phase was then extracted twice using dichloromethane. The organic phase was then combined and washed with saturated saline, dried over anhydrous sodium sulfate and concentrated under vacuum to yield 4-(3-(2-((R)-1-hydroxyethyl)-6-(phenylsulfonyl)imidazo[4,5-d]pyrrolo[2,3-b]pyridin-1(6H)-yl)pyrrolidin-1-carbonyl)benzonitrile as a brown oil (0.3 g, 76%). The product can be used directly in the next step without further purification. HRMS (ESI). m/z [M+H]⁺. C₂₈H₂₅N₆O₄S calculated value 541.1653, measured value 541.1643.

Step 2: 4-(3-(2-((R)-1-hydroxyethyl)-6-(phenylsulfonyl)imidazo[4,5-d]pyrrolo[2,3-b]pyridin-1(6H)-yl)pyrrolidin-1-carbonyl)benzonitrile (0.3 g, 0.6 mmol) was dissolved in a solvent mixture of 5 mL of tetrahydrofuran and 5 mL of methanol and stirred at room temperature for 5 h after the addition of 5 mL of 1 M sodium hydroxide. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate=1:1) to yield LXS02: 4-(3-(2-((R)-1-hydroxyethyl)imidazo[4,5-d]pyrrolo[2,3-b]pyridin-1(6H)-yl)pyrrolidin-1-carbonyl)benzonitrile (0.1 g, 45%). ¹ HNMR (300 MHz, DMSO-d₆): δ=11.96 (s, 1H), 8.87 (s, 1H), 8.21 (d, J=9.0 Hz, 2H), 8.10 (d, J=9.0 Hz, 2H), 7.66 (s, 1H), 6.88 (s, 1H), 4.28-4.69 (m, 1H), 3.81-3.92 (m, 2H), 3.74-3.79 (m, 1H), 3.30-3.45 (m, 2H), 1.93-2.15 (m, 2H), 1.49 (d, J=9.0 Hz, 3H) ppm; ¹³ C NMR (75 MHz, DMSO₆) δ 172.53, 148.69, 148.51, 142.16, 139.53, 132.09, 129.04, 127.91, 127.16, 120.75, 118.63, 115.61, 113.69, 99.39, 63.60, 58.45, 52.57, 47.13, 27.11, 22.82 ppm; HRMS (ESI): m/z [M+H]⁺. C₂H₂₁N₆O₂ calculated value 401.1721, measured value 401.1723.

### Example 3

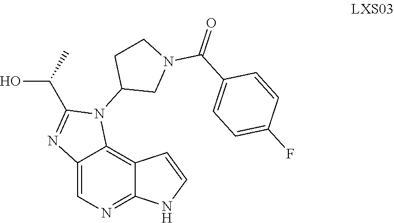 [SMILES: C[C@@H](O)c1nc2cnc3[nH]ccc3c2n1C1CCN(C(=O)c2ccc(F)cc2)C1]

### (4-Fluorophenyl)(3-(2-((R)-1-Hydroxyethyl)imidazo[4,5-d]pyrrolo[2,3-b]pyridin-1(6H)-yl)pyrrolidin-1-yl)methanone

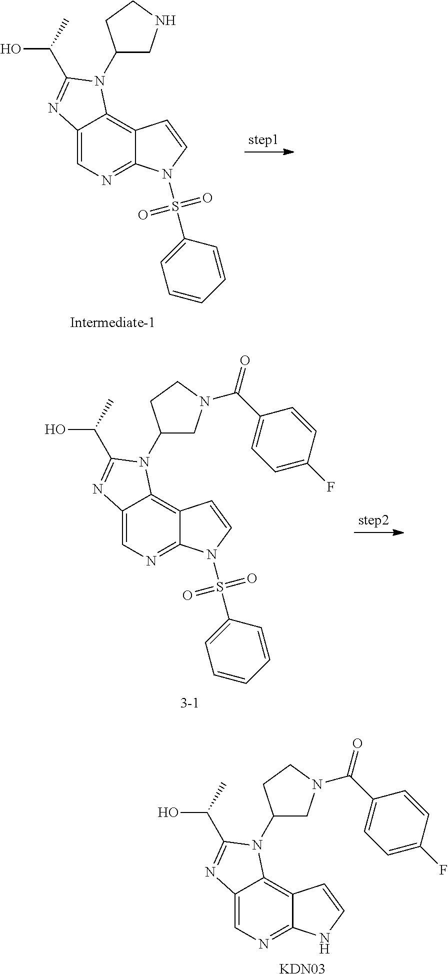

Step 1: Dissolve (1R)-1-(6-(phenylsulfonyl)-1-(pyrrolidin-3-yl)-1,6-dihydroimidazo[4,5-d]pyrrolo[2,3-b]pyridin-2-yl)ethanol (0.3 g, 0.7 mmol) in 10 mL of tetrahydrofuran, add DIPEA (0.2 g, 1.4 mmol) followed by slow dropwise addition of p-fluorobenzoyl chloride (0.2 g, 1.1 mmol). The reaction was monitored for completion by TLC. Saturated sodium bicarbonate was added until the reaction solution was weakly basic, and the organic phase was separated. The aqueous phase was then extracted twice using dichloromethane. The organic phase was then combined and washed with saturated saline, dried over anhydrous sodium sulfate and concentrated under vacuum to yield (4-fluorophenyl)(3-(2-((R)-1-hydroxyethyl)-6-(phenylsulfonyl)imidazo[4,5-d]pyrrolo[2,3-b]pyridin-1(6H)-yl)pyrrolidin-1-yl)methanone as a brown oil (0.3 g, 77%). The product can be used directly in the next step without further purification. HRMS (ESI): m/z [M+H]⁺. C₂₇H₂₅FN₅O₄S calculated value 534.1606, measured value 534.1619.

Step 2: (4-fluorophenyl)(3-(2-((R)-1-hydroxyethyl)-6-(phenylsulfonyl)imidazo[4,5-d]pyrrolo[2,3-b]pyridin-1(6H)-yl)pyrrolidin-1-yl)methanone (0.3 g, 0.6 mmol) was dissolved in a solvent mixture of 5 mL of tetrahydrofuran and 5 mL of methanol. After the addition of 5 mL of 1 M sodium hydroxide, the mixture was stirred at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate =1:1) to yield LXS03: (4-Fluorophenyl)(3-(2-((R)-1-Hydroxyethyl)imidazo[4,5-d]pyrrolo[2,3-b]pyridin-1(6H)-yl)pyrrolidin-1-yl)methanone (0.1 g, 45%). ¹ HNMR (3M) MHz, DMSO-d₆): δ=11.97 (s, 1H), 8.60 (d, J=6.0 Hz, 1H), 7.74-7-76 (m, 1H), 7.55 (d, J=15.0 Hz, 2H), 7.36 (t, J=9.0 Hz, 1H), 7.22 (t, J=9.0 Hz, 1H) Hz, 1H), 6.45 (d, J=48.0 Hz, 1H), 5.77 (s, 2H), 5.15-5.23 (m, 1H), 3.97-4.20 (m, 2H), 3.71-3.93 (m, 2H), 2.66-2.73 (m, 1H), 1.67 (d, J=6.0 Hz, 3H), 1.38 (t, J=37.5 Hz, 2H) ppm; ¹³ C NMR (75 MHz, DMSO-d₆) δ 172.58, 163.91, 148.83, 148.29, 142.54, 130.84, 129.03, 128.83, 127.29, 120.85, 115.61, 115.33, 99.57, 63.67, 58.49, 52.59, 47.83, 27.29, 22.89 ppm, HRMS (ESI): m/z [M+H]⁺. C₂₁H₂₁FNSO₂ calculated value 394.1674, measured value 394.1682.

### Example 4

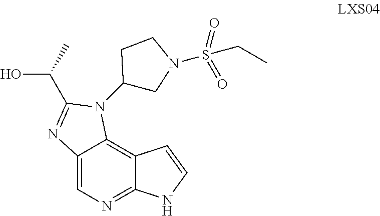 [SMILES: CCS(=O)(=O)N1CCC(n2c([C@@H](C)O)nc3cnc4[nH]ccc4c32)C1]

### (1R)-1-(1-(1-(ethylsulfonyl)pyrrolidin-3-yl)-1,6-dihydroimidazo[4,5-d]pyrrolo[2,3-b]pyridin-2-yl)ethan-1-ol

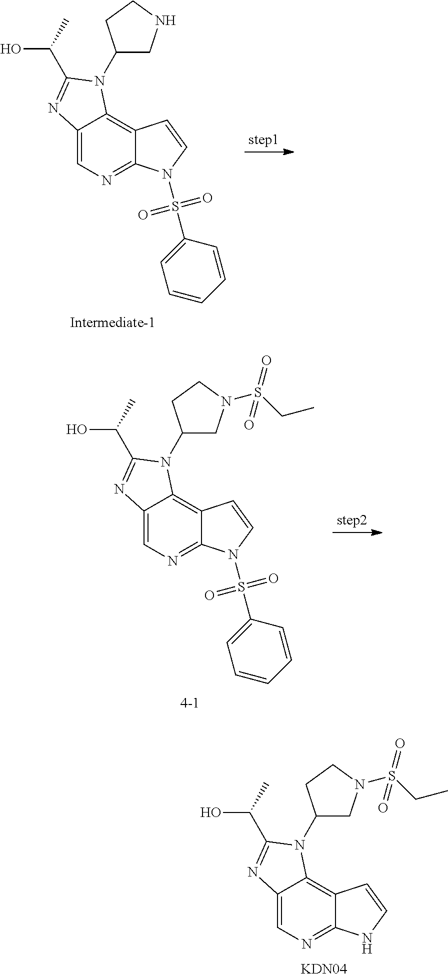

Step 1: Dissolve (1R)-1-(6-(phenylsulfonyl)-1-(pyrrolidin-3-yl)-1,6-dihydroimidazo[4,5-d]pyrrolo[2,3-b]pyridin-2-yl)ethanol (0.3 g, 0.7 mmol) in 10 mL of tetrahydrofuran, add DIPEA (0.2 g, 1.4 mmol) followed by the slow dropwise addition of ethyl sulfonyl chloride (0.2 g, 1.1 mmol). Then stir under reflux by heating for 3 h. The reaction was monitored for completion by TLC. Saturated sodium bicarbonate was then added until the reaction solution was weakly basic, and the organic phase was separated. The aqueous phase was then extracted twice using dichloromethane. The organic phase was then combined and washed with saturated saline, dried over anhydrous sodium sulfate and concentrated under vacuum to yield (1R)-1-(1-(1-(1-(ethylsulfonyl)pyrrolidin-3-yl)-6-(phenylsulfonyl)-1,6-dihydroimidazo[4,5-d]pyrrolo[2,3-b]pyridin-2-yl)ethanol as a yellow oil (0.3 g, 82%). The product can be used directly in the next step without further purification. HRMS (ESI): m/z [M+H]⁺. C₂₂H₂₆N₅O₅S₂ calculated value 504.1370, measured value 504.1389.

Step 2: (1R)-1-(1-(1-(1-(ethylsulfonyl)pyrrolidin-3-yl)-6-(phenylsulfonyl)-1,6-dihydroimidazo[4,5-d]pyrrolo[2,3-b]pyridin-2-yl) ethanol (0.3 g, 0.6 mmol) was dissolved in a mixture of 5 mL of tetrahydrofuran and 5 mL of methanol, and 5 mL of 1M sodium hydroxide was added and then stirred at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate=1:1) to yield LXS04: (1R)-1-(1-(1-(ethylsulfonyl)pyrrolidin-3-yl)-1,6-dihydroimidazo[4,5-d]pyrrolo[2,3-b]pyridin-2-yl)ethan-1-ol (0.2 g, 93%). ¹ HNMR (300 MHz, DMSO-d₆): δ=12.03 (s, 1H), 8.97 (s, 1H), 7.69 (s, 1H), 6.78 (s, 1H), 4.184.77 (m, 1H), 3.60-3.79 (m, 1H), 3.40-3.51 (m, 2H), 3.11-3.32 (m, 2H), 2.74-2.89 (m, 2H), 1.90-2.15 (m, 2H), 1.49 (d, J=6.0 Hz, 3H), 1.22 (t, J=9.0 Hz, 3H) ppm; ¹³ C NMR (75 MHz, DMSO-d₆) δ 148.77, 148.56, 142.77, 129.19, 127.31, 120.77, 115.68, 113.69, 99.48, 63.29, 57.53, 56.10, 51.11, 50.09, 26.20, 22.89, 2.69 ppm; HRMS (ESI): m/z [M+H]⁺. C₁₆H₂₂N₅O₃S calculated value 364.1438, measured value 364.1472.

### Example 5

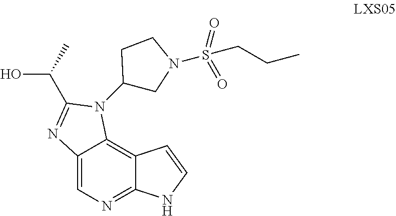 [SMILES: CCCS(=O)(=O)N1CCC(n2c([C@@H](C)O)nc3cnc4[nH]ccc4c32)C1]

### (1R)-1-(1-(1-(propylsulfonyl)pyrrolidin-3-yl)-1,6-dihydroimidazo[4,5-d]pyrrolo[2,3-b]pyridin-2-yl)ethan-1-ol

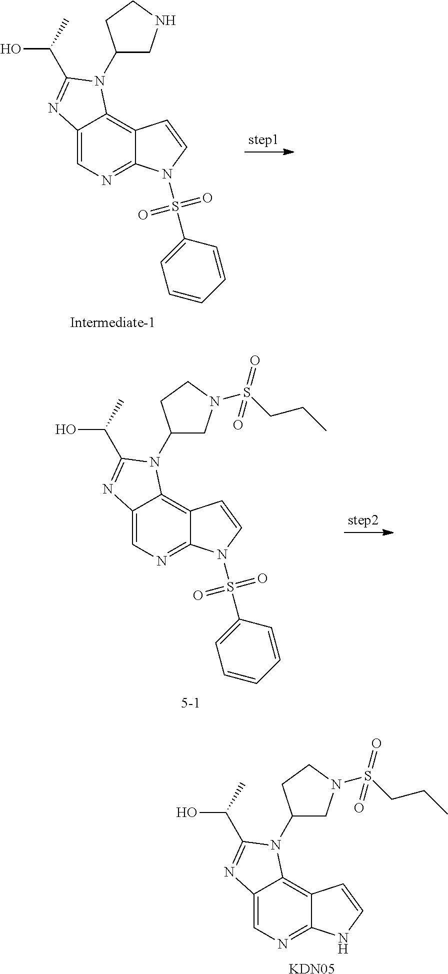

Step 1: Dissolve (1R)-1-6-(phenylsulfonyl)-1-(pyrrolidin-3-yl)-1,6-dihydroimidazo[4,5-d]pyrrolo[2,3-b]pyridin-2-yl)ethanol (0.3 g, 0.7 mmol) in 10 mL of tetrahydrofuran, add DIPEA (0.2 g, 1.4 mmol) followed by slow dropwise addition of propylsulfonyl chloride (0.2 g, 1.1 mmol). Then stir under reflux by heating for 3 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield (1R)-1-(1-(1-(1-(propylsulfonyl)pyrrolidin-3-yl)-6-(phenylsulfonyl)-1,6-dihydroimidazo[4,5-d]pyrrolo[2,3-b]pyridin-2-yl)ethanol as a yellow oil (0.3 g, 80%). The product can be used directly in the next step without further purification. HRMS (ESI): m/z [M+H]⁺. C₂₃H₂₈N₅O₅S₂ calculated value 518.1526, measured value 518.1536.

Step 2: (1R)-1-(1-(1-(1-(propylsulfonyl)pyrrolidin-3-yl)-6-(phenylsulfonyl)-1,6-dihydroimidazo[4,5-d]pyrrolo[2,3-b]pyridin-2-yl) ethanol (0.3 g, 0.6 mmol) was dissolved in a mixture of 5 mL of tetrahydrofuran and 5 mL of methanol, and 5 mL of 1M sodium hydroxide was added, and then stirred at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate=1:1) to yield LXS05: (1R)-1-(1-(1-(propylsulfonyl)pyrrolidin-3-yl)-1,6-dihydroimidazo[4,5-d]pyrrolo[2,3-b]pyridin-2-yl)ethan-1-ol (0.2 g, 91%). ¹ HNMR (300 MHz, DMSO-d₆): δ=11.93 (s, 1H), 8.77 (s, 1H), 7.58 (s, 1H), 6.67 (s, 1H), 4.21-4.68 (m, 1H), 3.51-3.73 (m, 1H), 3.21-3.28 (m, 2H), 3.18 (t, J=9.0 Hz, 2H), 2.79-2.99 (m, 2H), 1.83-2.25 (m, 2H), 1.52-1.68 (m, 2H), 1.49 (d, J=9.0 Hz, 3H), 0.90 (t, J=6.0 Hz, 3H) ppm; ¹³C NMR (75 MHz, DMSO-d₆) δ 148.66, 148.59, 142.19, 129.02, 127.15, 120.73, 115.61, 113.69, 99.31, 63.66, 60.40, 57.59, 56.19, 50.02, 26.28, 22.84, 13.33, 12.44 ppm; HRMS (ESI): m/z [M+H]⁺. C₁₇H₂₄NSO₃S calculated value 378.1594, measured value 378.1599.

### Example 6

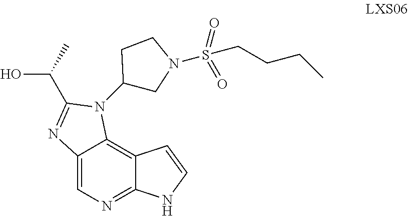 [SMILES: CCCCS(=O)(=O)N1CCC(n2c([C@@H](C)O)nc3cnc4[nH]ccc4c32)C1]

### (1R)-1-(1-(1-(butylsulfonyl)pyrrolidin-3-yl)-1,6-dihydroimidazo[4,5-d]pyrrolo[2,3-b]pyridin-2-yl)ethan-1-ol

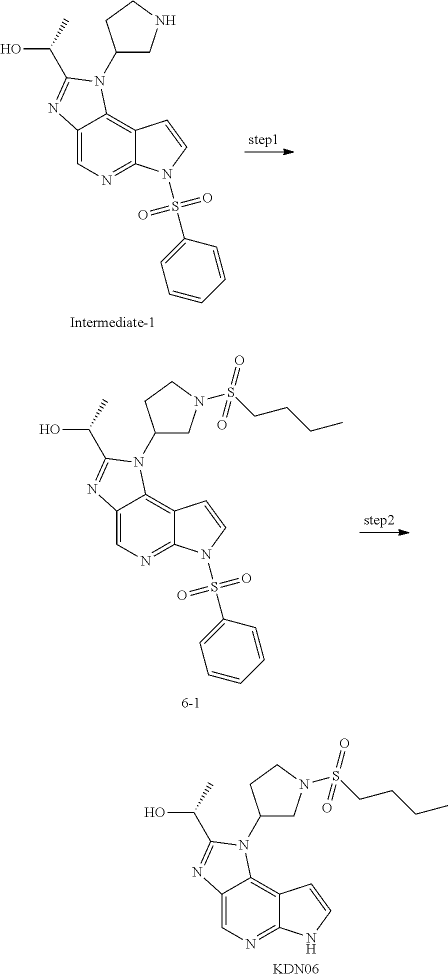

Step 1: Dissolve (1R)-1-(6-(phenylsulfonyl)-1-(pyrrolidin-3-yl)-1,6-dihydroimidazo[4,5-d]pyrrolo[2,3-b]pyridin-2-yl)ethanol (0.3 g, 0.7 mmol) in 10 mL of tetrahydrofuran, add DIPEA (0.2 g, 1.4 mmol) followed by the slow dropwise addition of butylsulfonyl chloride (0.2 g, 1.1 mmol). Then stir under reflux by heating for 3 h. The reaction was monitored for completion by TLC. Saturated sodium bicarbonate was then added until the reaction solution was weakly basic, and the organic phase was separated. The aqueous phase was then extracted twice using dichloromethane. The organic phases were then combined and washed with saturated saline, dried over anhydrous sodium sulfate and concentrated under vacuum to yield (1R)-1-(1-(1-(1-(butylsulfonyl)pyrrolidin-3-yl)-6-(phenylsulfonyl)-1,6-dihydroimidazo[4,5-d]pyrrolo[2,3-b]pyridin-2-yl)ethanol as a pale white oil (0.3 g, 77%). The product can be used directly in the next step without further purification. HRMS (ESI): m/z [M+H]⁺. C₂₄H₃₀N₅O₅S₂ calculated value 532.1683, measured value 532.1703.

Step 2: (1R)-1-(1-(1-(1-(butylsulfonyl)pyrrolidin-3-yl)-6-(phenylsulfonyl)-1,6-dihydroimidazo[4,5-d]pyrrolo[2,3-b]pyridin-2-yl) ethanol (0.3 g, 0.6 mmol) was dissolved in a mixture of 5 mL of tetrahydrofuran and 5 mL of methanol, and 5 mL of 1 M sodium hydroxide was added, and then stirred at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate=1:1) to yield LXS06: (1R)-1-(1-(1-(butylsulfonyl)pyrrolidin-3-yl)-1,6-dihydroimidazo[4,5-d]pyrrolo[2,3-b]pyridin-2-yl)ethan-1-ol (0.2 g, 91%). ¹ HNMR (300 MHz, DMSO-d₆): δ=12.03 (s, 1H), 8.47 (s, 1H), 7.68 (s, 1H), 6.87 (s, 1H), 4.41-4.62 (m, 1H), 3.50-3.79 (m, 1H), 3.11-3.22 (m, 2H), 3.10 (t, J=6.0 Hz, 2H), 2.77-2.80 (m, 2H), 1.93-2.15 (m, 2H), 1.51-1.61 (m, 2H), 1.48 (d, J=6.0 Hz, 3H), 1.29-1.31 (m, 2H), 0.93 (t, J=9.0 Hz, 3H) ppm; ¹³ C NMR (75 MHz, DMSO-d₆) δ 148.65, 148.51, 142.18, 129.04, 127.11, 120.79, 115.61, 99.39, 63.68, 57.93, 57.58, 56.17, 50.01, 26.28, 22.82, 21.93, 21.05, 13.88 ppm; HRMS (ESI): m/z [M+H]⁺. C₁₈H₂₆N₅O₃S calculated value 392.1751, measured value 392.1755.

### Example 7

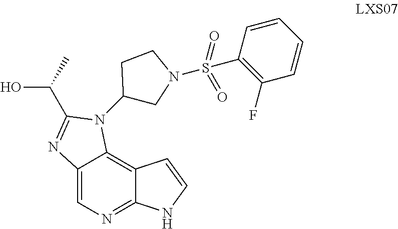 [SMILES: C[C@@H](O)c1nc2cnc3[nH]ccc3c2n1C1CCN(S(=O)(=O)c2ccccc2F)C1]

### (1R)-1-(1-(1-((2-fluorophenyl)sulfonyl)pyrrolidin-3-yl)-1,6-dihydroimidazo[4,5-d]pyrrolo[2,3-b]pyridin-2-yl)ethan-1-ol

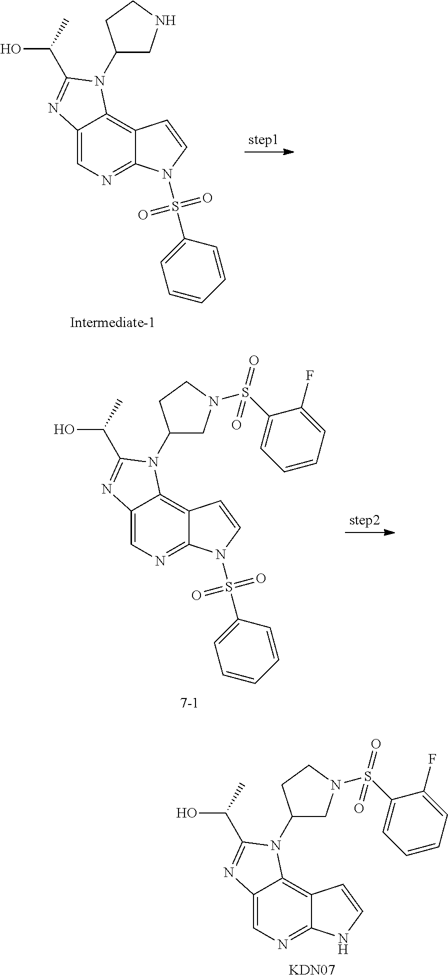

Step 1: Dissolve (1R)-1-(6-(phenylsulfonyl)-1-(pyrrolidin-3-yl)-1,6-dihydroimidazo[4,5-d]pyrrolo[2,3-b]pyridin-2-yl)ethanol (0.3 g, 0.7 mmol) in 10 mL of tetrahydrofuran, add DIPEA (0.2 g, 1.4 mmol) followed by the slow dropwise addition of o-fluorobenzenesulfonyl chloride (0.2 g, 1.1 mmol). Then stir under reflux by heating for 3 h. The reaction was monitored for completion by TLC. Saturated sodium bicarbonate was then added until the reaction solution was weakly basic, and the organic phase was separated. The aqueous phase was then extracted twice using dichloromethane. The organic phases were then combined and washed with saturated saline, dried over anhydrous sodium sulfate and concentrated under vacuum to yield (1R)-1-(1-(1-(1-(((2-fluorophenyl)sulfonyl)pyrrolidin-3-yl)-6-(phenylsulfonyl)-1,6-dihydroimidazo[4,5-d]pyrrolo[2,3-b]pyridin-2-yl)ethanol as a light yellow oil (0.3 g, 72%). The product can be used directly in the next step without further purification. HRMS (ESI): m/z [M+H]⁺. C₂₆H₂₅FN₅O₅S₂ calculated value 570.1276, measured value 570.1257.

Step 2: (1R)-1-(1-(1-(1-(((2-fluorophenyl)sulfonyl)pyrrolidin-3-yl)-6-(phenylsulfonyl)-1,6-dihydroimidazo[4,5-d]pyrrolo[2,3-b]pyridin-2-yl)ethanol (0.3 g, 0.5 mmol) was dissolved in a mixture of 5 mL of tetrahydrofuran and 5 mL of methanol, and 5 mL of 1 M sodium hydroxide was added, and then stirred at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate=1:1) to yield LXS07: (1R)-1-(1-(1-((2-fluorophenyl)sulfonyl)pyrrolidin-3-yl)-1,6-dihydroimidazo[4,5-d]pyrrolo[2,3-b]pyridin-2-yl)ethan-1-ol (0.1 g, 44%). ¹ HNMR (300 MHz, DMSO-d₆): δ=12.16 (s, 1H), 8.87 (s, 1H), 7.80-7.84 (m, 1H), 7.63-7.75 (m, 1H), 7.60 (s, 1H), 7.48-7.52 (m, 1H), 7.21-7.43 (m, 1H), 6.77 (s, 1H), 4.51-4.70 (m, 1H), 3.63-3.81 (m, 1H), 3.09-3.33 (m, 2H), 2.63-2.83 (m, 2H), 1.89-2.19 (m, 2H), 1.93 (d, J=9.0 Hz, 3H) ppm; ¹³ C NMR (75 MHz, DMSO-d₆) δ 158.22, 148.66, 148.57, 142.18, 133.53, 129.03, 128.91, 127.11, 126.33, 124.69, 120.74, 115.84, 115.64, 99.38, 63.69, 57.58, 55.70, 49.63, 26.24, 22.89 ppm; HRMS (ESI): m/z [M+H]⁺. C₂₀H₂₁FN₅O₃S calculated value 430.1344, measured value 430.1350.

### Example 8

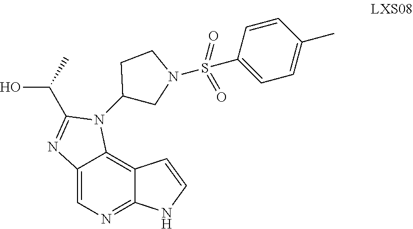 [SMILES: Cc1ccc(S(=O)(=O)N2CCC(n3c([C@@H](C)O)nc4cnc5[nH]ccc5c43)C2)cc1]

### (1R)-1-(1-(1-tosylpyrrolidin-3-yl)-1,6-dihydroimidazo[4,5-d]pyrrolo[2,3-b]pyridin-2-yl)ethan-1-ol

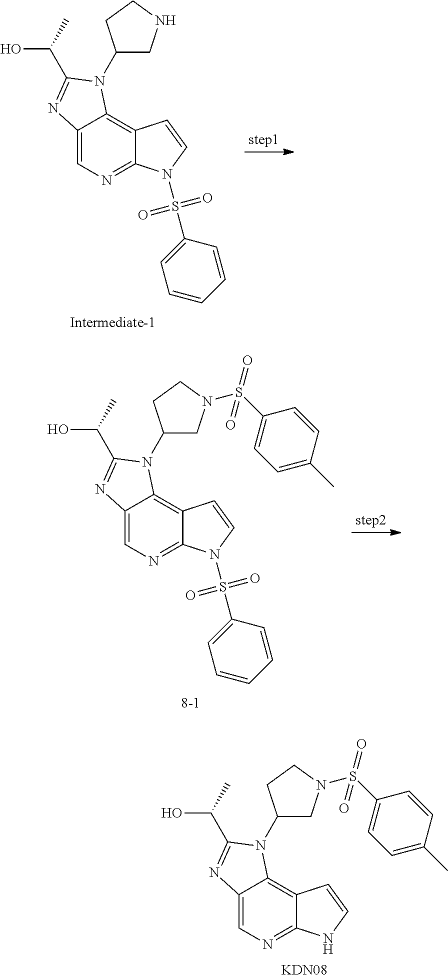

Step 1: Dissolve (1R)-1-(6-(phenylsulfonyl)-1-(pyrrolidin-3-yl)-1,6-dihydroimidazo[4,5-d]pyrrolo[2,3-b]pyridin-2-yl)ethanol (0.3 g, 0.7 mmol) in 10 mL of tetrahydrofuran, add DIPEA (0.2 g, 1.4 mmol) and slowly add p-toluenesulfonyl chloride (0.2 g, 1.4 mmol) dropwise, then raise the temperature while maintaining reflux and stir for 3 h. Monitor the reaction for completion by TLC. Saturated sodium bicarbonate was then added until the reaction solution was weakly basic, and the organic phase was separated. The aqueous phase was then extracted twice using dichloromethane. The organic phases were then combined and washed with saturated saline, dried over anhydrous sodium sulfate and concentrated under vacuum to yield (1R)-1-(6-(phenylsulfonyl)-1-(1-toluenesulfonylpyrrolidin-3-yl)-1,6-dihydroimidazo[4,5-d]pyrrolo[2,3-b]pyridin-2-yl)ethanol as a yellow oil (0.3 g, 73%). The product can be used directly in the next step without further purification. HRMS (EST): m/z [M+H]⁺. C₂₇H₂₈N₅O₅S₂ calculated value 566.1526, measured value 566.1523.

Step 2: (1R)-1-(6-(phenylsulfonyl)-1-(1-toluenesulfonylpyrrolidin-3-yl)-1,6-dihydroimidazo[4,5-d]pyrrolo[2,3-b]pyridin-2-yl) ethanol (0.3 g, 0.5 mmol) was dissolved in a solvent mixture of 5 mL tetrahydrofuran and 5 mL methanol. Then, 5 mL of 1 M sodium hydroxide was added and the mixture was stirred at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate=1:1) to yield LXS08: (1R)-1-(1-(1-tosylpyrrolidin-3-yl)-1,6-dihydroimidazo[4,5-d]pyrrolo[2,3-b]pyridin-2-yl)ethan-1-ol (0.2 g, 89%). ¹ HNMR (300 MHz, DMSO-d₆): δ=11.95 (s, 1H), 8.84 (s, 1H), 7.74 (d, J=7.5 Hz, 2H), 7.68 (s, 1H), 7.40 (d, J=7.5 Hz, 2H), 6.87 (s, 1H), 4.53-4.68 (m, 1H), 3.55-3.81 (m, 1H), 3.10-3.25 (m, 2H), 2.79-2.89 (m, 2H), 2.34 (s, 3H), 1.91-2.25 (m, 2H), 1.43 (d, J=4.5 Hz, 3H) ppm; ¹³ C NMR (75 MHz, DMSO-d₆) δ 148.63, 148.59, 143.33, 142.17, 137.66, 129.39, 129.00, 128.33, 127.16, 120.71, 115.65, 99.39, 63.64, 57.58, 55.74, 49.63, 26.23, 22.81, 21.39 ppm. HRMS (EST): m/z [M+H]. C₂₁H₂₄N₅O₃S calculated value 426.1594, measured value 426.1596.

### Example 9

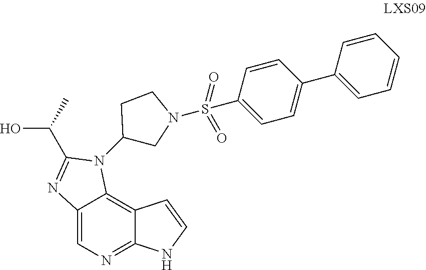 [SMILES: C[C@@H](O)c1nc2cnc3[nH]ccc3c2n1C1CCN(S(=O)(=O)c2ccc(-c3ccccc3)cc2)C1]

### (1R)-1-(1-(1-([1,1′-biphenyl]-4-ylsulfonyl)pyrrolidin-3-yl)-1,6-dihydroimidazo[4,5-d]pyrrolo[2,3-b]pyridin-2-yl)ethan-1-ol

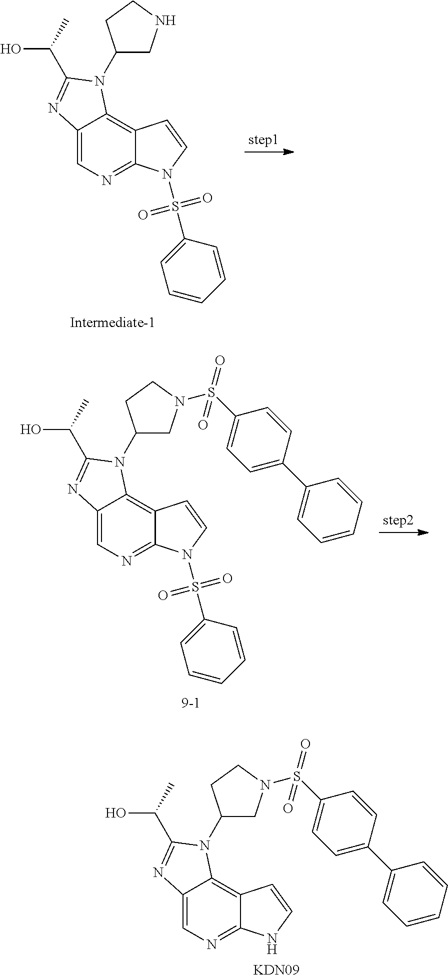

Step 1: Dissolve (1R)-1-6-(phenylsulfonyl)-1-(pyrrolidin-3-yl)-1,6-dihydroimidazo[4,5-d]pyrrolo[2,3-b]pyridin-2-yl)ethanol (0.3 g, 0.7 mmol) in 10 mL of tetrahydrofuran, add DIPEA (0.2 g, 1.4 mmol) and slowly add p-phenylbenzenesulfonyl chloride (0.3 g, 1.1 mmol) dropwise. Raise the temperature while maintaining reflux and stir for 3 h. Monitor the reaction for completion by TLC. Saturated sodium bicarbonate was then added until the reaction solution was weakly basic, and the organic phase was separated. The aqueous phase was then extracted twice using dichloromethane. The organic phases were then combined and washed with saturated saline, dried over anhydrous sodium sulfate and concentrated under vacuum to yield (1R)-1-(1-(1-(1-([[1,1′-biphenyl]-4-yl]sulfonyl)pyrrolidin-3-yl)-6-(phenylsulfonyl)-1,6-dihydroimidazo[4,5-d]pyrrolo[2,3-b]pyridin-2-yl)ethanol as a yellow oil (0.3 g, 66%), 66%). The product can be used directly in the next step without further purification. HRMS (ESI): m/z [M+H]⁺. C₃₂H₃₀N₅O₅S₂ calculated value 628.1683, measured value 628.1686.

Step 2: (1R)-1-(1-(1-(1-([[1,1′-Biphenyl]-4-yl sulfonyl)pyrrolidin-3-yl)-6-(phenylsulfonyl)-1,6-dihydroimidazo[4,5-d ]pyrrolo[2,3-b]pyridin-2-yl)ethanol (0.3 g, 0.5 mmol) was dissolved in a solvent mixture of 5 mL tetrahydrofuran and 5 mL methanol. After adding 5 mL of 1 M sodium hydroxide, the mixture was stirred at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate =1:1) to yield LXS09: (1R)-1-(1-(1-([1,1′-biphenyl]-4-ylsulfonyl)pyrrolidin-3-yl)-1,6-dihydroimidazo[4,5-d]pyrrolo[2,3-b]pyridin-2-yl)ethan-1-ol (0.2 g, 86%). ¹ HNMR (300 MHz, DMSO-d₆): δ=12.35 (s, 1H), 8.74 (s, 1H), 7.88-7.92 (m, 4H), 7.67 (s, 1H), 7.45-7.52 (m, 4H), 7.28-7.41 (m, 1H), 6.87 (s, 1H), 4.43-4.71 (m, 1H), 3.52-3.79 (m, 1H), 3.11-3.35 (m, 2H), 2.77-2.88 (m, 2H), 1.90-2.15 (m, 2H), 1.49 (d, J=6.0 Hz, 3H) ppm; ¹³ C NMR (75 MHz, DMSO-d₆) δ 148.69, 148.53, 142.19, 140.88, 138.62, 133.06, 129.21, 129.04, 127.93, 127.88, 127.11, 127.10, 120.73, 115.63, 99.38, 63.63, 57.58, 55.70, 49.62, 26.25, 22.81 ppm: HRMS (EST): m/z [M+H]⁺. C₂₆H₂₆N₅O₃S calculated value 488.1751, measured value 488.1757.

### Example 10

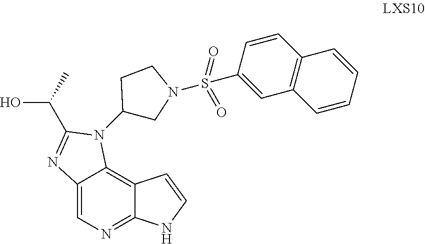 [SMILES: C[C@@H](O)c1nc2cnc3[nH]ccc3c2n1C1CCN(S(=O)(=O)c2ccc3ccccc3c2)C1]

### (1R)-1-(1-(1-(naphthalen-2-ylsulfonyl)pyrrolidin-3-yl)-1,6-dihydroimidazo[4,5-d]pyrrolo[2,3-b]pyridin-2-yl)ethan-1-ol

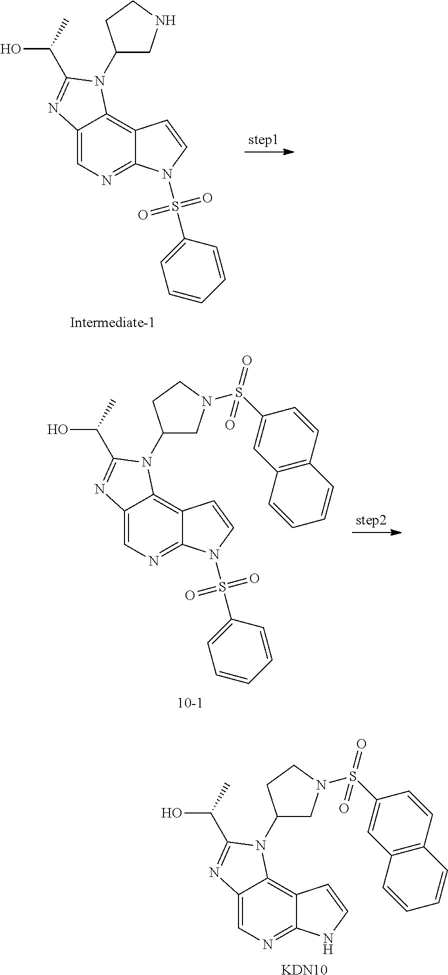

Step 1: Dissolve (1R)-1-(6-(phenylsulfonyl)-1-(pyrrolidin-3-yl)-1,6-dihydroimidazo[4,5-d]pyrrolo[2,3-b]pyridin-2-yl)ethanol (0.3 g, 0.7 mmol) in 10 mL of tetrahydrofuran, add DIPEA (0.2 g, 1.4 mmol) followed by slow dropwise addition of naphthalenesulfonyl chloride (0.3 g, 1.1 mmol). Raise the temperature to maintain reflux and stir for 3 h. Monitor the reaction for completion by TLC. Saturated sodium bicarbonate was then added until the reaction solution was weakly basic, and the organic phase was separated. The aqueous phase was then extracted twice using dichloromethane. The organic phases were then combined and washed with saturated saline, dried over anhydrous sodium sulfate and concentrated under vacuum to yield (1R)-1-(1-(1-(1-naphthalen-2-yl sulfonyl)pyrrolidin-3-yl)-6-(phenylsulfonyl)-1,6-dihydroimidazo[4,5-d]pyrrolo[2,3-b]pyridin-2-yl)ethanol as a pale yellow oil (0.3 g, 68%). The product can be used directly in the next step without further purification. HRMS (ESI); m/z [M+H]⁺. C₃₀H₂₈N₅O₅S₂ calculated value 602.1526, measured value 602.1540.

Step 2: (1R)-1-(1-(1-(1-naphthalen-2-yl sulfonyl)pyrrolidin-3-yl)-6-(benzenesulfonyl)-1,6-dihydroimidazo[4,5-d]pyrrolo[2,3-b]pyridin-2-yl) ethanol (0.3 g, 0.5 mmol) was dissolved in a mixture of 5 mL tetrahydrofuran and 5 mL methanol, and 5 mL of 1 M sodium hydroxide was added, and then stirred at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate=1:1) to yield LXS10: (1R)-1-(1-(1-(naphthalen-2-ylsulfonyl)pyrrolidin-3-yl)-1,6-dihydroimidazo[4,5-d]pyrrolo[2,3-b]pyridin-2-yl)ethan-1-ol (0.2 g, 87%). ¹ HNMR (300 MHz, DMSO-d₆): δ=12.04 (s, 1H), 8.87 (s, 1H), 8.80 (s, 1H), 8.30-8.41 (m, 1H), 8.03-8.15 (m, 1H), 8.00 (d, J=7.5 Hz, 2H), 7.59 (d, J=7.5 Hz, 2H=7.5 Hz, 2H), 7.45 (s, 1H), 6.87 (s, 1H), 4.55-4.69 (m, 1H), 3.45-3.91 (m, 1H), 3.15-3.35 (m, 2H), 2.69-2.74 (m, 2H), 1.96-2.28 (m, 2H), 1.49 (d, J=6.0 Hz, 3H) ppm; ¹³ C NMR (75 MHz, DMSO-d₆) δ 148.62, 148.51, 142.17, 137.03, 136.71, 134.11, 129.44, 129.09, 128.11, 127.11, 126.25, 126.04, 123.41, 120.74, 115.60, 99.38, 63.69, 57.57, 55.76, 49.60, 26.22, 22.81 ppm; HRMS (ESI): m/z [M+H]⁺. C₂₄H₂₄N₅O₃S calculated value 462.1594, measured value 462.1607.

### Example 11

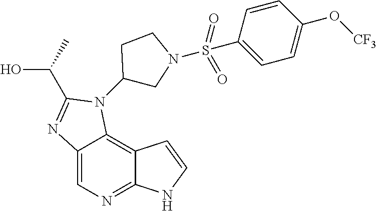 [SMILES: C[C@@H](O)c1nc2cnc3[nH]ccc3c2n1C1CCN(S(=O)(=O)c2ccc(OC(F)(F)F)cc2)C1]

### (1R)-1-(1-(1-((4-(trifluoromethoxy)phenyl)sulfonyl)pyrrolidin-3-yl)-1,6-dihydroimidazo[4,5-d]pyrrolo[2,3-b]pyridin-2-yl)ethan-1-ol

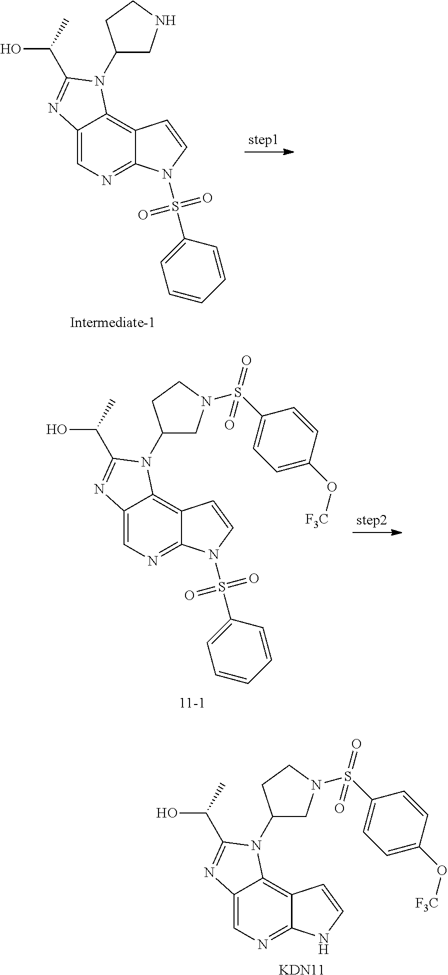

Step 1: Dissolve (1R)-1-(6-(phenylsulfonyl)-1-(pyrrolidin-3-yl)-1,6-dihydroimidazo[4,5-d]pyrrolo[2,3-b]pyridin-2-yl)ethanol (0.3 g, 0.7 mmol) in 10 mL of tetrahydrofuran, add DIPEA (0.2 g, 1.4 mmol) and then slowly add p-trifluoromethoxy-benzenesulfonyl chloride (0.3 g, 1.1 mmol) dropwise, Raise the temperature to maintain reflux and stir for 3 h. Monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield (1R)-1-(6-(phenylsulfonyl)-1-(1-((4-(trifluoromethoxy)phenyl)sulfonyl)pyrrolidin-3-yl)-1,6-dihydroimidazo[4,5-d]pyrrolo[2,3-b]pyridin-2-yl)ethanol as a yellow oil (0.3 g, 65%). The product can be used directly in the next step without further purification. HRMS (ESI): m/z [M+H]⁺. C₂₇H₂₅F₃N₅O₆S₂ calculated value 636.1193, measured value 636.1195.

Step 2: (1R)-1-(6-(phenylsulfonyl)-1-(1-((4-(trifluoromethoxy)phenyl)sulfonyl)pyrrolidin-3-yl)-1,6-dihydroimidazo[4,5-d]pyrrolo[2,3-b]pyridin-2-yl)ethanol (0.3 g, 0.5 mmol) was dissolved in a solvent mixture of 5 mL tetrahydrofuran and 5 mL methanol, and 5 mL of 1 M sodium hydroxide was added and then stirred at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS11: (1R)-1-(1-(1-((4-(trifluoromethoxy)phenyl)sulfonyl)pyrrolidin-3-yl)-1,6-dihydroimidazo[4,5-d]pyrrolo[2,3-b]pyridin-2-yl)ethan-1-ol (0.2 g, 86%). ¹ HNMR (300 MHz, DMSO-d₆): δ=12.15 (s, 1H), 8.79 (s, 1H), 7.64 (d, J=7.5 Hz, 2H), 7.59 (s, 1H), 7.12 (d, J=7.5 Hz, 2H), 6.74 (s, 1H), 4.33-4.58 (m, 1H), 3.65-3.83 (m, 1H), 3.15-3.31 (m, 2H), 2.74-2.86 (m, 2H), 1.95-2.35 (m, 2H), 1.46 (d, J=9.0 Hz, 3H) ppm; ¹³ C NMR (75 MHz, DMSO-d₆) δ 153.88, 148.61, 148.59, 142.10, 132.00, 129.79, 129.04, 127.17, 126.11, 120.73, 115.61, 114.67, 99.38, 63.69, 57.58, 55.70, 49.66, 26.25, 22.81 ppm: HRMS (ESI): m/z [M+H]⁺. C₂₁H₂₁F₃N₅O₄S calculated value 496.1261, measured value 496.1262.

### Example 12

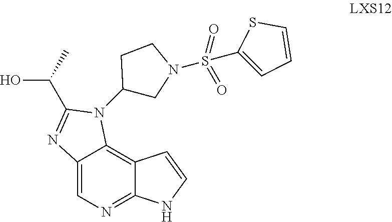 [SMILES: C[C@@H](O)c1nc2cnc3[nH]ccc3c2n1C1CCN(S(=O)(=O)c2cccs2)C1]

### (1R)-1-(1-(1-(thiophen-2-ylsulfonyl)pyrrolidin-3-yl)-1,6-dihydroimidazo[4,5-d]pyrrolo[2,3-b]pyridin-2-yl)ethan-1-ol

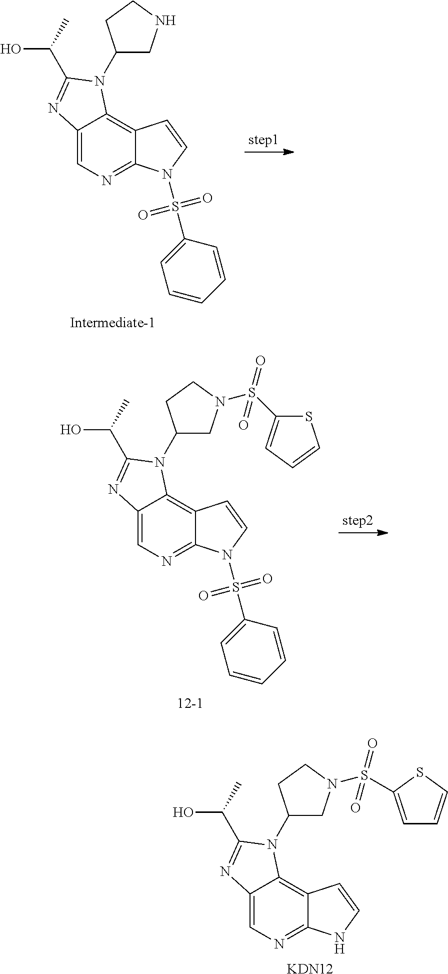

Step 1: Dissolve (1R)-1-(6-(phenylsulfonyl)-1-(pyrrolidin-3-yl)-1,6-dihydroimidazo[4,5-d]pyrrolo[2,3-b]pyridin-2-yl)ethanol (0.3 g, 0.7 mmol) in 10 mL of tetrahydrofuran, add DIPEA (0.2 g, 1.4 mmol) followed by the slow dropwise addition of thiophene sulfonyl chloride (0.2 g, 1.1 mmol). Raise the temperature to maintain reflux and stir for 3 h. Monitor the reaction for completion by TLC. Saturated sodium bicarbonate was then added until the reaction solution was weakly basic, and the organic phase was separated. The aqueous phase was then extracted twice using dichloromethane. The organic phases were then combined and washed with saturated saline, dried over anhydrous sodium sulfate and concentrated under vacuum to yield (1R)-1-(6-(phenylsulfonyl)-1-(1-(thiophen-2-yl sulfonyl)pyrrolidin-3-yl)-1,6-dihydroimidazo[4,5-d]pyrrolo[2,3-b]pyridin-2-yl)ethanol as a yellow oil (0.3 g, 74%). The product can be used directly in the next step without further purification. HRMS (ESI): m/z [M+H]⁺. C₂₄H₂₄N₅O₅S₃ calculated value 558.0934, measured value 558.0949.

Step 2: (1R)-1-(6-(phenylsulfonyl)-1-(1-(thiophen-2-yl sulfonyl)pyrrolidin-3-yl)-1,6-dihydroimidazo[4,5-d]pyrrolo[2,3-b]pyridin-2-yl) ethanol (0.3 g, 0.5 mmol) was dissolved in a mixture of 5 mL of tetrahydrofuran and 5 mL of methanol, and 5 mL of 1 M sodium hydroxide was added and then stirred at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS12: (1R)-1-(1-(1-(thiophen-2-ylsulfonyl)pyrrolidin-3-yl)-1,6-dihydroimidazo[4,5-d]pyrrolo[2,3-b]pyridin-2-yl)ethan-1-ol (0.1 g, 45%). ¹ HNMR (3AX MHz, DMSO-d₆): δ=12.02 (s, 1H), 8.84 (s, 1H), 7.70 (s, 1H), 7.20 (d, J=7.5 Hz, 1H), 6.96 (d, J=9.0 Hz, 2H), 6.77 (s, 1H), 4.51-4.69 (m, 1H), 3.57-3.83 (m, 1H), 3.17-3.31 (m, 2H), 2.72-2.85 (m, 2H), 1.93-2.31 (m, 2H), 1.07 (d, J=9.0 Hz, 3H) ppm; ¹³ C NMR (75 MHz, DMSO-d₆) δ 148.61, 148.57, 142.11, 129.09, 127.21, 127.11, 126.33, 120.73, 115.60, 99.31, 63.61, 57.59, 55.74, 49.62, 26.22, 22.81 ppm; HRMS (ESI): m/z [M+H]⁺. C₁₈H₂₀N₅O₃S₂ calculated value 418.1002, measured value 418.1012.

### Example 13

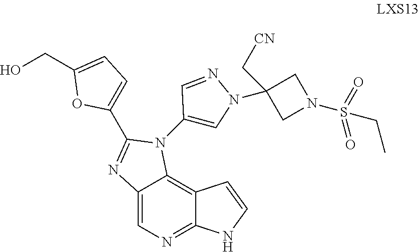 [SMILES: CCS(=O)(=O)N1CC(CC#N)(n2cc(-n3c(-c4ccc(CO)o4)nc4cnc5[nH]ccc5c43)cn2)C1]

### (1-(Ethylsulfonyl)-3-(4-(2-(5-(hydroxymethyl)furan-2-yl)imidazo[4,5-d]pyrrolo[2,3-b]pyridin-1(6H)-yl)-1H-pyrazol-1-yl)azetidin-3-yl)acetonitrile

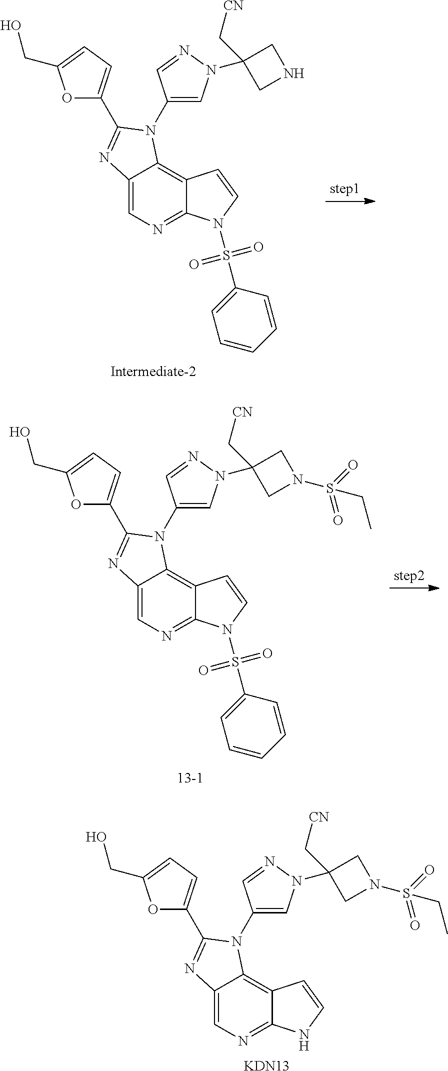

Step 1: Dissolve 2-(3-(4-(2-(5-(hydroxymethyl)furan-2-yl)-6-(phenylsulfonyl)imidazo[4,5-d]pyrrolo[2,3-b]pyridin-1(6H)-yl)-1H-pyrazol-1-yl)azetidin-3-yl)acetonitrile (0.3 g, 0.5 mmol) in 10 mL of tetrahydrofuran, add DIPEA (0.2 g, 1.0 mmol) followed by slow addition of ethylsulfonyl chloride (0.1 g, 0.8 mmol) dropwise. Raise the temperature to maintain reflux and stir for 3 h. Monitor the reaction for completion by TLC. Saturated sodium bicarbonate was then added until the reaction solution was weakly basic, and the organic phase was separated. The aqueous phase was then extracted twice using dichloromethane. The organic phases were then combined and washed with saturated saline, dried over anhydrous sodium sulfate and concentrated under vacuum to yield 2-(1-(ethylsulfonyl)-3-(4-(2-(5-(hydroxymethyl)furan-2-yl)-6-(phenylsulfonyl)imidazo[4,5-d]pyrrolo[2,3-b]pyridin-1(6H)-yl)-1H-pyrazol-1-yl)azetidin-3-yl)acetonitrile as a yellow oil (0.2 g, 57%). The product can be used directly in the next step without further purification. HRMS (ESI): m/z [M+H]⁺. C₂₉H₂₇N₈O₆S₂ calculated value 647.1489, measured value 647.1482.

Step 2: 2-(1-(Ethylsulfonyl)-3-(4-(2-(5-(hydroxymethyl)furan-2-yl)-6-(phenylsulfonyl)imidazo[4,5-d]pyrrolo[2,3-b]pyridin-1(6H)-yl)-1H-pyrazol-1-yl)azetidin-3-yl)acetonitrile (0.2 g, 0.3 mmol) was dissolved in a mixture of solvent of 5 mL tetrahydrofuran and 5 mL methanol, 5 mL of 1 M sodium hydroxide was added and then stirred at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS13: 2-(1-(ethylsulfonyl)-3-(4-(2-(5-(hydroxymethyl)furan-2-yl)imidazo[4,5-d]pyrrolo[2,3-b]pyridin-1(6H)-yl)-1H-pyrazol-1-yl)azetidin-3-yl)acetonitrile (0.1 g, 64%). ¹ HNMR (300 MHz, DMSO-d₆): δ=11.94 (s, 1H), 8.90 (s, 1H), 8.69 (s, 1H), 8.14 (s, 1H), 7.37 (s, 1H), 6.41 (s, 2H), 5.95 (s, 1H), 5.43 (t, J=6.0 Hz, 1H), 4.62 (d, J=9.0 Hz, 2H), 4.47 (d, J=6.0 Hz, 2H), 4.34 (d, J=9.0 Hz, 2H), 3.78 (s, 2H), 3.25-3.32 (m, 2H), 1.28 (t, J=7.5 Hz, 3H) ppm; ¹³ C NMR (75 MHz, DMSO-d₆) δ 153.88, 151.11, 148.67, 142.16, 141.57, 130.77, 129.79, 129.04, 127.12, 120.74, 117.77, 115.65, 107.93, 104.05, 100.53, 99.38, 59.23, 57.39, 51.60, 50.81, 23.20, 2.62 ppm: HRMS (ESI): m/z [M+H]⁺. C₂₃H₂₃N₅O₄S calculated value 507.1557, measured value 507.1549.

### Example 14

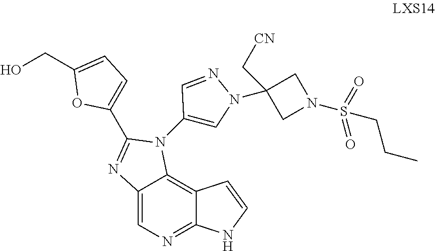 [SMILES: CCCS(=O)(=O)N1CC(CC#N)(n2cc(-n3c(-c4ccc(CO)o4)nc4cnc5[nH]ccc5c43)cn2)C1]

### 2-(1-(Propylsulfonyl)-3-(4-(2-(5-(hydroxymethyl)furan-2-yl)imidazo[4,5-d]pyrrolo[2,3-b]pyridin-1(6H)-yl)-1H-pyrazol-1-yl)azetidin-3-yl)acetonitrile

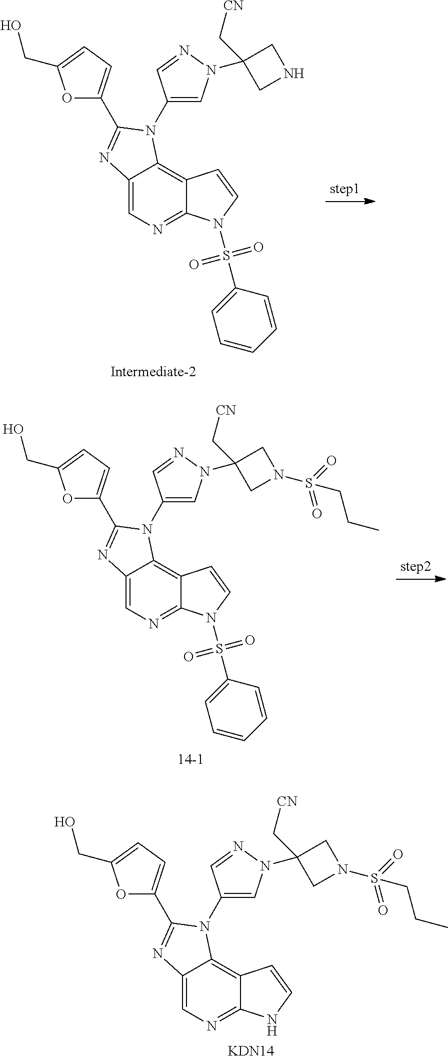

Step 1: Dissolve 2-(3-(4-(2-(5-(hydroxymethyl)furan-2-yl)-6-(phenylsulfonyl)imidazo[4,5-d]pyrrolo[2,3-b]pyridin-1(6H)-yl)-1H-pyrazol-1-yl)azetidin-3-yl)acetonitrile (0.3 g, 0.5 mmol) in 10 mL of tetrahydrofuran. After the addition of DIPEA (0.2 g, 1.0 mmol), propylsulfonyl chloride (0.1 g, 0.8 mmol) was added slowly dropwise. Raise the temperature to maintain reflux and stir for 3 h. Monitor the reaction for completion by TLC. Saturated sodium bicarbonate was then added until the reaction solution was weakly basic, and the organic phase was separated. The aqueous phase was then extracted twice using dichloromethane. The organic phases were then combined and washed with saturated saline, dried over anhydrous sodium sulfate and concentrated under vacuum to yield 2-(1-(propylsulfonyl)-3-(4-(2-(5-(hydroxymethyl)furan-2-yl)-6-(phenylsulfonyl)imidazo[4,5-d]pyrrolo[2,3-b]pyridin-1(6H)-yl)-1H-pyrazol-1-yl)azetidin-3-yl)acetonitrile as a yellow oil (0.3 g, 84%). The product can be used directly in the next step without further purification. HRMS (ESI): m/z [M+H]⁺. C₃₀H₂₉N₅O₆S₂ calculated value 661.1646, measured value 661.1649.

Step 2: 2-(1-(propylsulfonyl)-3-(4-(2-(5-(hydroxymethyl)furan-2-yl)-6-(phenylsulfonyl)imidazo[4,5-d]pyrrolo[2,3-b]pyridin-1(6H)-yl)-1H-pyrazol-1-yl)azetidin-3-yl)acetonitrile (0.3 g, 0.5 mmol) was dissolved in a solvent mixture of 5 mL of tetrahydrofuran and 5 mL of methanol. Then, 5 ml of 1 M sodium hydroxide was added and the mixture was stirred at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS14: 2-(1-(propylsulfonyl)-3-(4-(2-(5-(hydroxymethyl)furan-2-yl)imidazo[4,5-d]pyrrolo[2,3-b]pyridin-1(6H)-yl)-1H-pyrazol-1-yl)azetidin-3-yl)acetonitrile (0.1 g, 42%). ¹ HNMR (300 MHz, DMSO-d₆): δ=11.91 (s, 1H), 8.87 (s, 1H), 8.66 (s, 1H), 8.11 (s, 1H), 7.34 (s, 1H), 6.38 (s, 2H), 5.93 (s, 1H), 5.40 (t, J=6.0 Hz, 1H), 4.58 (d, J=9.0 Hz, 2H), 4.45 (d, J=3.0 Hz, 2H), 4.31 (d, J=9.0 Hz, 2H), 3.75 (s, 2H), 3.24 (t, J=7.5 Hz, 2H), 1.69-1.77 (m, 2H), 1.18-1.30 (m, 2H), 1.00 (t, J=7.5 Hz, 3H) ppm; ¹³ C NMR (75 MHz, DMSO-d₆) δ 157.85, 145.73, 143.25, 143.07, 139.10, 135.78, 135.68, 134.04, 128.63, 124.75, 119.74, 117.03, 113.11, 109.22, 104.52, 96.44, 58.75, 57.15, 56.11, 50.29, 27.30, 16.89, 13.16 ppm; HRMS (ESI): m/z [M+H]⁺. C₂₄H₂₅N₈O₄S calculated value 521.1714, measured value 521.1695.

### Example 15

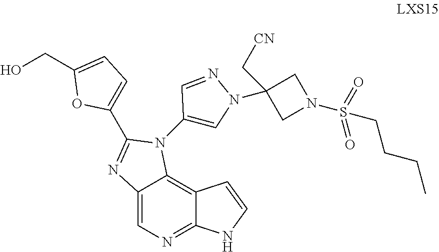 [SMILES: CCCCS(=O)(=O)N1CC(CC#N)(n2cc(-n3c(-c4ccc(CO)o4)nc4cnc5[nH]ccc5c43)cn2)C1]

### 2-(1-(Butylsulfonyl)-3-(4-(2-(5-(hydroxymethyl)furan-2-yl)imidazo[4,5-d]pyrrolo[2,3-b]pyridin-1(6H)-yl)-1H-pyrazol-1-yl)azetidin-3-yl)acetonitrile

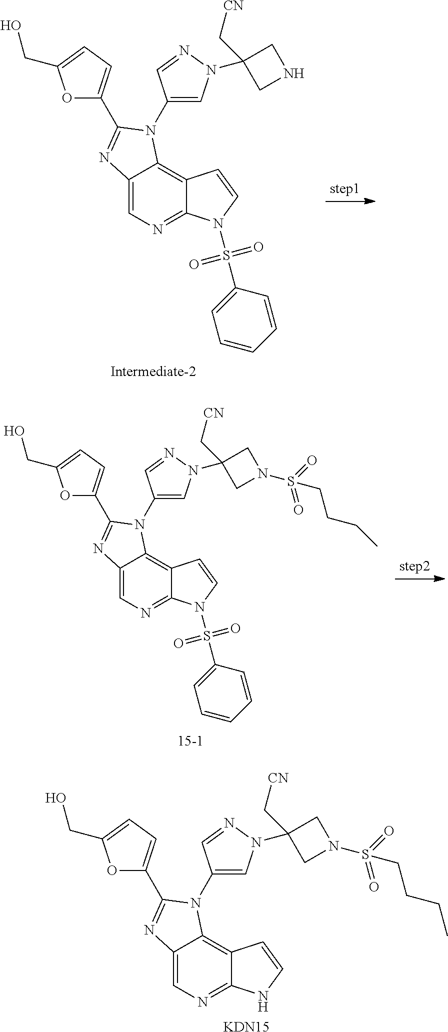

Step 1: Dissolve 2-(3-(4-(2-(5-(hydroxymethyl)furan-2-yl)-6-(phenylsulfonyl)imidazo[4,5-d]pyrrolo[2,3-b]pyridin-1(6H)-yl)-1H-pyrazol-1-yl)azetidin-3-yl)acetonitrile (0.3 g, 0.5 mmol) in 10 mL of tetrahydrofuran. After the addition of DIPEA (0.2 g, 1.0 mmol), butylsulfonyl chloride (0.1 g, 0.8 mmol) was added slowly dropwise. Raise the temperature to maintain reflux and stir for 3 h. Monitor the reaction for completion by TLC. Saturated sodium bicarbonate was then added until the reaction solution was weakly basic, and the organic phase was separated. The aqueous phase was then extracted twice using dichloromethane. The organic phases were then combined and washed with saturated saline, dried over anhydrous sodium sulfate and concentrated under vacuum to yield 2-(1-(butylsulfonyl)-3-(4-(2-(5-(hydroxymethyl)furan-2-yl)-6-(phenylsulfonyl)imidazo[4,5-d]pyrrolo[2,3-b ]pyridin-1(6H)-yl)-1H-pyrazol-1-yl)azetidin-3-yl)acetonitrile as a yellow oil (0.2 g, 55%). The product can be used directly in the next step without further purification. HRMS (ESI): m/z [M+H]⁺. C₃₁H₃₁N₈O₆S₂ calculated value 675.1802, measured value 675.1811.

Step 2: 2-(1-(Butylsulfonyl)-3-(4-(2-(5-(hydroxymethyl)furan-2-yl)-6-(phenylsulfonyl)imidazo[4,5-d]pyrrolo[2,3-b ]pyridin-1(6H)-yl)-1H-pyrazol-1-yl)azetidin-3-yl)acetonitrile (0.2 g, 0.3 mmol) was dissolved in a solvent mixture of 5 mL of tetrahydrofuran and 5 mL of methanol, 5 ml of 1 M sodium hydroxide was added and the mixture was then stirred at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS15: 2-(1-(butylsulfonyl)-3-(4-(2-(5-(hydroxymethyl)furan-2-yl)imidazo[4,5-d]pyrrolo[2,3-b]pyridin-1(6H)-yl)-1H-pyrazol-1-yl)azetidin-3-yl)acetonitrile (0.1 g, 63%). ¹ HNMR (300 MHz, DMSO-d₆): δ=11.95 (s, 1H), 8.90 (s, 1H), 8.69 (s, 1H), 8.14 (s, 1H), 7.37 (s, 1H), 6.41 (s, 2H), 5.96 (s, 1H), 5.44 (t, J=6.0 Hz, 1H), 4.62 (d, J=9.0 Hz, 2H), 4.48 (d, J=6.0 Hz, 2H), 4.35 (d, J=9.0 Hz, 2H), 3.78 (s, 2H), 3.29 (t, J=7.5 Hz, 2H), 2.53 (s, 2H), 1.66-1.76 (m, 2H), 1.38-1.50 (m, 2H), 0.92 (t, J=7.5 Hz, 3H) ppm: ¹ C NMR (75 MHz, DMSO-d₆) δ 157.86, 145.75, 143.27, 143.09, 139.10, 135.80, 135.69, 134.05, 128.65, 124.76, 119.77, 117.06, 113.13, 109.23, 104.54, 96.46, 58.78, 57.16, 56.13, 48.45, 27.33, 25.08, 21.36, 13.94 ppm: HRMS (ESI): m/z [M+H]⁺. C₂₅H₂₇N₅O₄S calculated value 535.1870, measured value 535.1870.

### Example 16

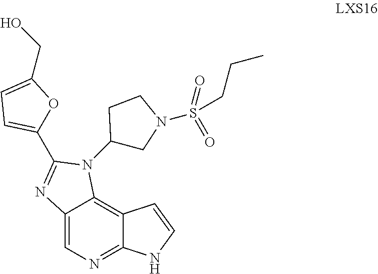 [SMILES: CCCS(=O)(=O)N1CCC(n2c(-c3ccc(CO)o3)nc3cnc4[nH]ccc4c32)C1]

### (5-(1-(1-(thiophen-2-ylsulfonyl)pyrrolidin-3-yl)-1,6-dihydroimidazo[4,5-d]pyrrolo[2,3-b]pyridin-2-yl)furan-2-yl)methanol

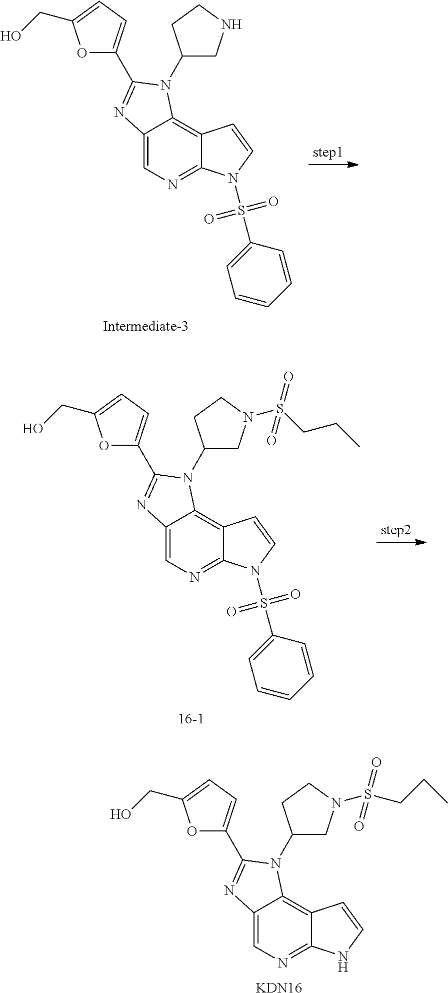

Step 1: Dissolve 5-(6-(phenylsulfonyl)-1-(pyrrolidin-3-yl)-1,6-dihydroimidazo[4,5-d]pyrrolo[2,3-b]pyridin-2-yl)furan-2-methanol (0.3 g, 0.6 mmol) in 10 mL of tetrahydrofuran, add DIPEA (0.2 g, 1.2 mmol) followed by slow dropwise addition of propylsulfonyl chloride (0.1 g, 0.9 mmol). Raise the temperature to maintain reflux and stir for 3 h. Monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield (5-(6-(phenylsulfonyl)-1-(1-(propylsulfonyl)pyrrolidin-3-yl)-1,6-dihydroimidazo[4,5-d]pyrrolo[2,3-b]pyridin-2-yl furan-2-yl) methanol as a yellow oil (0.2 g, 54%). The product can be used directly in the next step without further purification. HRMS (ESI): m/z [M+H]⁺. C₂₆H₂₈N₅O₆S₂ calculated value 570.1476, measured value 570.1488.

Step 2: Dissolve (5-(6-(phenylsulfonyl)-1-(1-(propylsulfonyl)pyrrolidin-3-yl)-1,6-dihydroimidazo[4,5-d]pyrrolo[2,3-b]pyridin-2-yl furan-2-yl) methanol (0.2 g, 0.4 mmol) in a solvent mixture of 5 mL of tetrahydrofuran and 5 mL of methanol. Then, add 5 mL of 1 M sodium hydroxide. Stir at room temperature for 5 h. The reaction was monitored for completion by TLC. Saturated sodium bicarbonate was then added until the reaction solution was weakly basic, and the organic phase was separated. The aqueous phase was then extracted twice using dichloromethane. The organic phases were then combined and washed with saturated saline, dried over anhydrous sodium sulfate and concentrated under vacuum, and the residue was purified using a silica gel column (petroleum ether:ethyl acetate=1:1) to yield LXS16: (5-(1-(1-(thiophen-2-ylsulfonyl)pyrrolidin-3-yl)-1,6-dihydroimidazo[4,5-d]pyrrolo[2,3-b]pyridin-2-yl)furan-2-yl)methanol (0.1 g, 66%). ¹HNMR (300 MHz, DMSO-d₆): δ=12.11 (s, 1H), 8.69 (s, 1H), 7.62 (s, 1H), 7.20 (d, J=3.0 Hz, 1H), 6.83 (s, 1H), 6.64 (d, J=3.0 Hz, 1H), 5.58-5.92 (m, 1H), 5.52 (t, J=6.64 (d, J=3.0 Hz, 1H), 5.58-5.92 (m, 1H), 5.52 (t, J=6.0 Hz, 1H), 4.59 (d, J=6.0 Hz, 2H), 4.04 (t, J=10.5 Hz, 1H), 3.79-3.87 (m, 2H), 3.53-3.62 (m, 1H), 3.24-3.29 (m, 2H), 2.69-2.83 (m, 1H), 1.76-1.88 (m, 2H), 1.07 (t, J=7.5 Hz, 3H) ppm; ¹³ C NMR (75 MHz, DMSO-d₆) δ 158.09, 144.98, 143.27, 143.04, 136.34, 135.19, 132.29, 125.05, 114.29, 109.37, 105.02, 98.97, 56.21, 54.67, 49.36, 48.97, 46.90, 30.36, 16.90, 13.35 ppm: HRMS (ESI): m/z. [M+H]⁺. C₂₀H₂₄N₅O₄S calculated value 430.1544, measured value 430.1550.

### Example 17

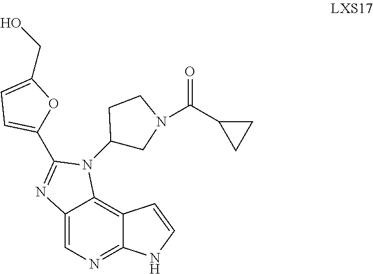 [SMILES: O=C(C1CC1)N1CCC(n2c(-c3ccc(CO)o3)nc3cnc4[nH]ccc4c32)C1]

### Cyclopropyl(3-(2-(5-(hydroxymethyl)furan-2-yl)imidazo[4,5-d]pyrrolo[2,3-b]pyridin-1(6H)-yl)pyrrolidin-1-yl) methanone

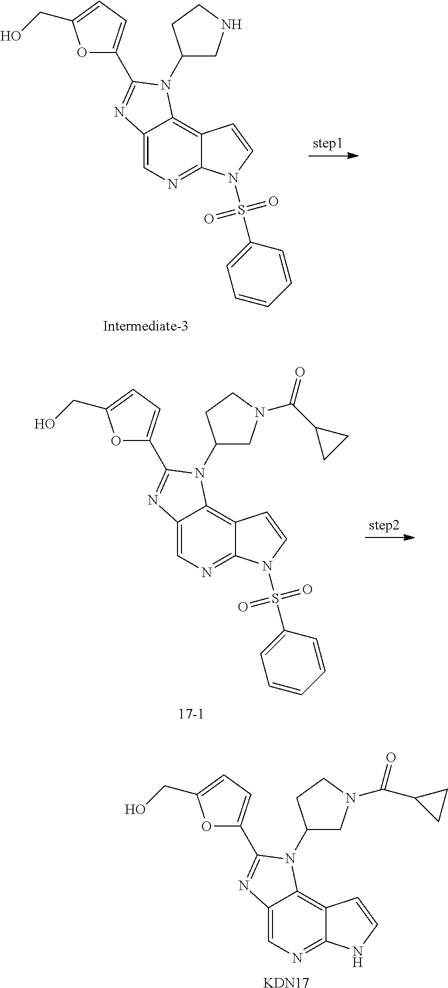

Step 1: Dissolve (5-(6-(phenylsulfonyl)-1-(pyrrolidin-3-yl)-1,6-dihydroimidazo[4,5-d]pyrrolo[2,3-b]pyridin-2-yl)furan-2-methanol (0.3 g, 0.6 mmol) in 10 mL of tetrahydrofuran, add DIPEA (0.2 g, 1.2 mmol) followed by slow dropwise addition of cyclopropanecarbonyl chloride (0.1 g, 0.9 mmol). Raise the temperature to maintain reflux and stir for 3 h. Monitor the reaction for completion by TLC. Saturated sodium bicarbonate was then added until the reaction solution was weakly basic, and the organic phase was separated. The aqueous phase was then extracted twice using dichloromethane. The organic phases were then combined and washed with saturated saline, dried over anhydrous sodium sulfate and concentrated under vacuum to yield cyclopropyl (3-(2-(5-(hydroxymethyl)furan-2-yl)-6-(phenylsulfonyl)imidazo[4,5-d]pyrrolo[2,3-b]pyridin-1(6H)-yl)pyrrolidin-1-yl)methanone as a brown oil (0.2 g, 58%). The product can be used directly in the next step without further purification. HRMS (ESI): m/z [M+H]⁺. C₂₇H₂₆N₅O₅S calculated value 532.1649, measured value 532.1661.

Step 2: Cyclopropyl (3-(2-(5-(hydroxymethyl)furan-2-yl)-6-(phenylsulfonyl)imidazo[4,5-d]pyrrolo[2,3-b]pyridin-1(6H)-yl)pyrrolidin-1-yl) methanone (0.2 g, 0.4 mmol) was dissolved in a mixture of 5 mL of tetrahydrofuran and 5 mL of methanol. Then, 5 mL of 1 M sodium hydroxide was added and the mixture was stirred for 5 h at room temperature. The reaction was monitored for completion by TLC. Saturated sodium bicarbonate was then added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS17: Cyclopropyl(3-(2-(5-(hydroxymethyl)furan-2-yl)imidazo[4,5-d]pyrrolo[2,3-b]pyridin-1(6H)-yl)pyrrolidin-1-yl) methanone (0.1 g, 68%). ¹ HNMR (300 MHz, DMSO-d₆): δ=12.10 (s, 1H), 8.69 (d, J=3.0 Hz, 1H), 7.60 (t, J=4.5 Hz, 1H), 7.22 (dd, J₁=9.0 Hz, J₂=3.0 Hz, 1H), 6.61 (t, J=4.5 Hz, 1H), 6.46 (d, J=9.0 Hz, 1H), 5.84-6.03 (m, 1H), 5.50 (t, J=4.5 Hz, 1H), 4.57 (d, J=6.0 Hz, 2H), 4.20-4.42 (m, 1H), 4.02-4.12 (m, 1H), 3.81-3.95 (m, 1H), 2.65-2.86 (m, 1H), 1.72-2.01 (m, 1H), 1.25 (s, 2H), 0.84 (t, J=7.5 Hz, 2H), 0.74 (d, J=9.0 Hz, 2H) ppm; ¹³ C NMR (75 MHz, DMSO-d₆) δ 171.92, 158.10, 158.05, 144.98, 143.47, 142.97, 136.41, 135.27, 132.40, 125.22, 114.32, 109.43, 105.09, 105.01, 98.67, 56.20, 45.58, 12.55, 7.68 ppm; HRMS (ESI): m/z [M+H]⁺. C₂₁H₂₂N₅O₃ calculated value 392.1717, measured value 392.1727.

### Example 18

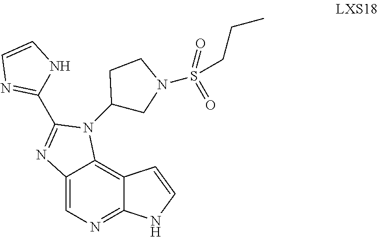 [SMILES: CCCS(=O)(=O)N1CCC(n2c(-c3ncc[nH]3)nc3cnc4[nH]ccc4c32)C1]

### 2-(1H-imidazol-2-yl)-1-(1-(propylsulfonyl)pyrrolidin-3-yl)-1,6-dihydroimidazo[4,5-d]pyrrolo[2,3-b]pyridine

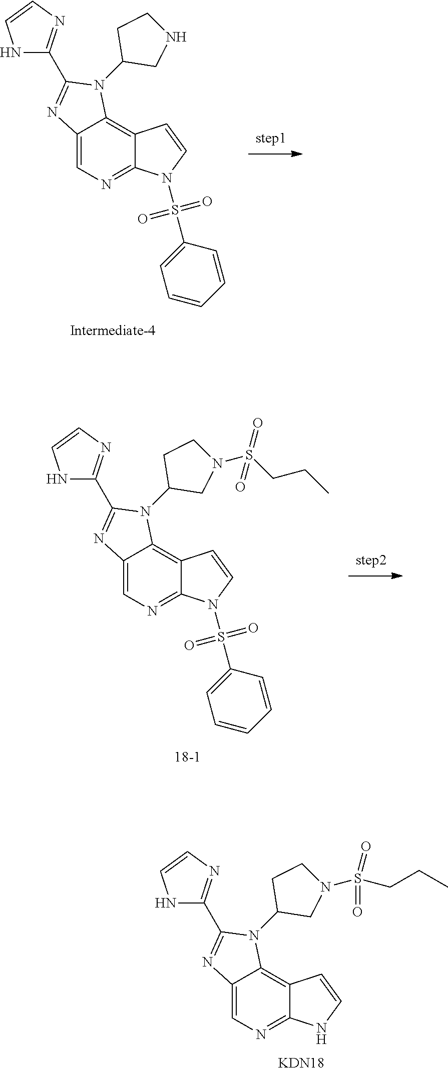

Step 1: Dissolve 2-(1H-imidazol-2-yl)-6-(phenylsulfonyl)-1-(pyrrolidin-3-yl)-1,6-dihydroimidazo[4,5-d]pyrrolo[2,3-b]pyridine (0.3 g, 0.7 mmol) in 10 mL of tetrahydrofuran, add DIPEA (0.2 g, 1.4 mmol) followed by the slow dropwise addition of propylsulfonyl chloride (0.2 g, 1.0 mmol). Raise the temperature to maintain reflux and stir for 3 h. Monitor the reaction for completion by TLC. Saturated sodium bicarbonate was then added until the reaction solution was weakly basic, and the organic phase was separated. The aqueous phase was then extracted twice using dichloromethane. The organic phases were then combined and washed with saturated saline, dried over anhydrous sodium sulfate and concentrated under vacuum to yield 2-(1H-imidazol-2-yl)-6-(phenylsulfonyl)-1-(1-(propylsulfonyl)pyrrolidin-3-yl)-1,6-dihydroimidazo[4,5-d]pyrrolo[2,3-b]pyridine as a yellow oil (0.2 g, 54%). The product can be used directly in the next step without further purification. HRMS (ESI): m/z [M+H]⁺. C₂₄H₂₆N₇O₄S₂ calculated value 540.1482, measured value 540.1485.

Step 2: 2-(1H-imidazol-2-yl)-6-(phenylsulfonyl)-1-(1-(propylsulfonyl)pyrrolidin-3-yl)-1,6-dihydroimidazo[4,5-d]pyrrolo[2,3-b]pyridine (0.2 g, 0.4 mmol) was dissolved in a solvent mixture of 5 mL tetrahydrofuran and 5 mL methanol. 5 mL of 1 M sodium hydroxide was then added and the mixture was stirred at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhdrous sodium sulfate and concentrated under vacuum, and the residue was purified using a silica gel column (petroleum ether:ethyl acetate=1:1) to yield LXS18: 2-(1H-imidazol-2-yl)-1-(1-(propylsulfonyl)pyrrolidin-3-yl)-1,6-dihydroimidazo[4,5-d]pyrrolo[2,3-b]pyridine (0.1 g, 68%). ¹ HNMR (300 MHz, DMSO-d₆): δ=13.34 (s, 1H), 12.09 (d, J=9.0 Hz, 1H), 8.72 (d, J=6.0 Hz, 1H), 7.58 (t, J=12.0 Hz, 2H), 7.41 (s, 1H), 7.25 (d, J=6.0 Hz, 2H), 6.83 (s, 1H), 4.08 (s, 2H), 3.83-3.97 (m, 3H), 3.20-3.32 (m, 2H), 2.79 (t, J=9.0 Hz, 1H), 2.11 (s, 2H), 1.76-1.86 (m, 2H), 1.25 (s, 1H), 1.08 (t, J=6.0 Hz, 3H) ppm; ¹³ C NMR (75 MHz, DMSO-d₆) δ 157.04, 148.61, 142.46, 138.11, 129.09, 127.92, 122.74, 120.71, 115.15, 99.49, 60.42, 56.83, 56.19, 50.01, 36.27, 13.20, 12.62 ppm; HRMS (ESI): m/z [M+H]⁺. C₁₈H₂₂N₇O₂S calculated value 400.1550, measured value 400.1561.

### Example 19

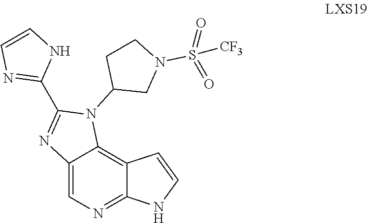 [SMILES: O=S(=O)(N1CCC(n2c(-c3ncc[nH]3)nc3cnc4[nH]ccc4c32)C1)C(F)(F)F]

### 2-(1H-imidazol-2-yl)-1-(1-((trifluoromethyl)sulfonyl)pyrrolidin-3-yl)-1,6-dihydroimidazo[4,5-d]pyrrolo[2,3-b]pyridine

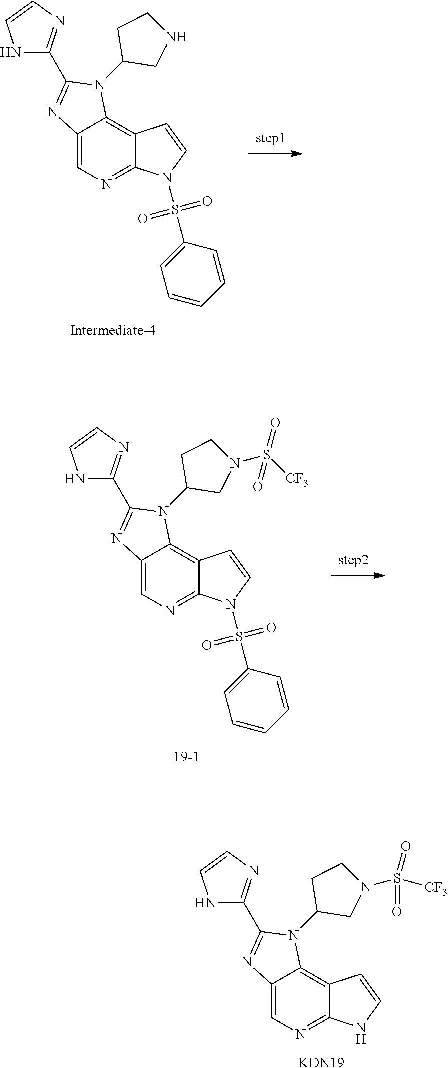

Step 1: Dissolve 2-(1H-imidazol-2-yl)-6-(phenylsulfonyl)-1-(pyrrolidin-3-yl)-1,6-dihydroimidazo[4,5-d]pyrrolo[2,3-b]pyridine (0.3 g, 0.7 mmol) in 10 mL of tetrahydrofuran, add DIPEA (0.2 g, 1.4 mmol) followed by the slow dropwise addition of trifluoromethylsulfonyl chloride (0.2 g, 1.0 mmol). Raise the temperature to maintain reflux and stir for 3 h. Monitor the reaction for completion by TLC. Saturated sodium bicarbonate was then added until the reaction solution was weakly basic, and the organic phase was separated. The aqueous phase was then extracted twice using dichloromethane. The organic phases were then combined and washed with saturated saline, dried over anhydrous sodium sulfate and concentrated under vacuum to yield 2-(1H-imidazol-2-yl)-6-(phenylsulfonyl)-1-(1-(((trifluoromethyl)sulfonyl)pyrrolidin-3-yl)-1,6-dihydroimidazo[4,5-d]pyrrolo[2,3-b]pyridine as a yellow oil (0.3 g, 77%). The product can be used directly in the next step without further purification. HRMS (ESI): m/z [M+H]⁺. C₂₂H₁₉F₃N₇O₄S₂ calculated value 566.0887, measured value 566.0894.

Step 2: 2-(1H-imidazol-2-yl)-6-(phenylsulfonyl)-1-(1-(((trifluoromethyl)sulfonyl)pyrrolidin-3-yl)-1,6-dihydroimidazo[4,5-d]pyrrolo[2,3-b]pyridine (0.3 g, 0.5 mmol) was dissolved in a solvent mixture of 5 mL tetrahydrofuran and 5 mL methanol. Then, 5 ml of 1M sodium hydroxide was added and then the mixture was stirred at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate=1:1) to yield LXS19: 2-(1H-imidazol-2-yl)-1-(1-((trifluoromethyl)sulfonyl)pyrrolidin-3-yl)-1,6-dihydroimidazo[4,5-d]pyrrolo[2,3-b]pyridine (0.1 g, 44%). ¹ HNMR (300 MHz, DMSO-d₆): δ=13.36 (s, 1H), 12.16 (s, 1H), 8.72 (s, 1H), 7.66 (s, 1H), 7.41 (s, 1H), 7.26 (s, 2H), 6.65 (s, 1H), 4.17 (d, J=9.0 Hz, 2H), 3.90 (d, J=6.0 Hz, 1H), 2.57-2.72 (m, 1H), 1.25 (s, 2H) ppm; ¹³ C NMR (75 MHz, DMSO-d₆) δ 144.80, 142.52, 138.37, 136.04, 134.54, 132.85, 125.37, 119.82, 104.82, 98.39, 48.52 ppm: HRMS (ESI): m/z [M+H]⁺. C₁₆H₁₅F₃N₇O₂S calculated value 426.0955, measured value 426.0959.

### Example 20

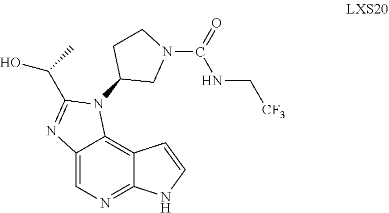 [SMILES: C[C@@H](O)c1nc2cnc3[nH]ccc3c2n1[C@H]1CCN(C(=O)NCC(F)(F)F)C1]

### (R)-3-(2-((R)-1-Hydroxyethyl)imidazo[4,5-d]pyrrolo[2,3-b]pyridin-1(6H)-yl)-N-(2,2,2-trifluoroethyl)pyrrolidine-1-carboxamide

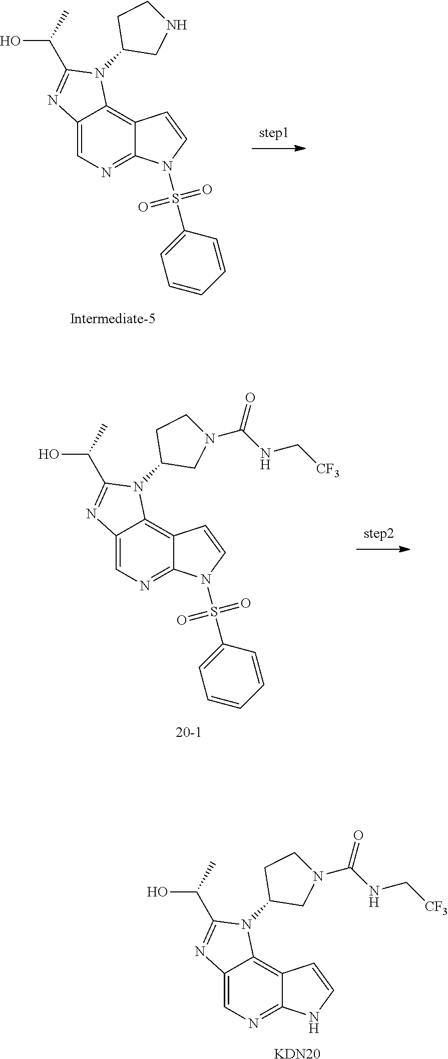

Step 1: Dissolve (R)-1-(6-(phenylsulfonyl)-1-((R-pyrrolidin-3-yl)-1,6-dihydroimidazo[4,5-d]pyrrolo[2,3-b]pyridin-2-yl) ethanol (0.3 g, 0.7 mmol) in 10 mL of tetrahydrofuran, add carbonyl diimidazole (0.2 g, 1.4 mmol) and slowly add trifluoromethyl ethylamine (0.2 g, 1.4 mmol) dropwise. Then, stir at room temperature for 3 h and monitor the reaction for completion by TLC. Saturated sodium bicarbonate was then added until the reaction solution was weakly basic, and the organic phase was separated. The aqueous phase was then extracted twice using dichloromethane. The organic phases were then combined and washed with saturated saline, dried over anhydrous sodium sulfate and concentrated under vacuum to yield (R)-3-(2-((R)-1-hydroxyethyl)-6-(phenylsulfonyl)imidazo[4,5-d]pyrrolo[2,3-b]pyridin-1(6H)-yl)-N-(2,2,2-trifluoroethyl)pyrrolidine-1-carboxamide as a pale yellow oil (0.2 g, 51%).). The product can be used directly in the next step without further purification. HRMS (ESI); m/z [M+H]. C₂₃H₂₄F₃N₆O₄S calculated value 537.1526, measured value 537.1511.

Step 2: Dissolve (R)-3-(2-((R)-1-hydroxyethyl)-6-(phenylsulfonyl)imidazo[4,5-d]pyrrolo[2,3-b]pyridin-1(6H)-yl)-N-(2,2,2-trifluoroethyl)pyrrolidine-1-carboxamide (0.2 g, 0.4 mmol) in a mixture of 5 mL of tetrahydrofuran and 5 mL of methanol, add 5 mL of 1 M sodium hydroxide and stir the mixture at room temperature for 5 h. The reaction was monitored for completion by TLC. Saturated sodium bicarbonate was then added until the reaction solution was weakly basic, and the organic phase was separated. The aqueous phase was then extracted twice using dichloromethane. The organic phases were then combined and washed with saturated saline, dried over anhydrous sodium sulfate and concentrated under vacuum, and the residue was purified using a silica gel column (petroleum ether:ethyl acetate=1:1) to yield LXS20: (R)-3-(2-((R)-1-Hydroxyethyl)imidazo[4,5-d]pyrrolo[2,3-b]pyridin-1(6H)-yl)-N-(2,2,2-trifluoroethyl)pyrrolidine-1-carboxamide (0.1 g, 44%). ¹ HNMR (300 MHz, DMSO-d₆): δ=11.96 (s, 1H), 8.62 (s, 1H), 7.51 (s, 1H), 7.09 (s, 1H), 6.37 (s, 1H), 5.80 (d, J=6.0 Hz, 2H), 5.22 (t, J=6.0 Hz, 1H), 3.87-3.96 (m, 5H), 3.49 (d, J=6.0 Hz, 1H), 2.64-2.76 (m, 11H), 2.43 (s, 1H), 1.68 (d, J=3.0 Hz, 3H), 1.53 (s, 1H), 1.26 (s, 3H), 0.89 (d, J=6.0 Hz, 2H) ppm; ¹³ C NMR (75 MHz, DMSO-d₆) δ 155.73, 148.66, 148.57, 142.76, 129.09, 127.12, 124.28, 120.70, 115.65, 99.38, 63.64, 57.99, 54.17, 48.74, 41.88, 26.60, 22.82 ppm: HRMS (ESI): m/z [M+H]⁺. C₁₇H₂₀F₃N₆O₂ calculated value 397.1594, measured value 397.1603.

### Example 21

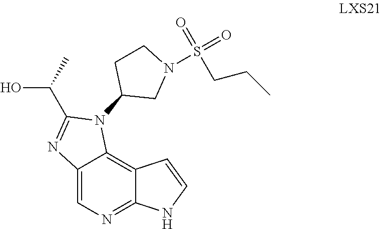 [SMILES: CCCS(=O)(=O)N1CC[C@H](n2c([C@@H](C)O)nc3cnc4[nH]ccc4c32)C1]

### (R)-1-(1-((R)-1-(Propylsulfonyl)pyrrolidin-3-yl)-1,6-dihydroimidazo[4,5-d]pyrrolo[2,3-b]pyridin-2-yl)ethanol

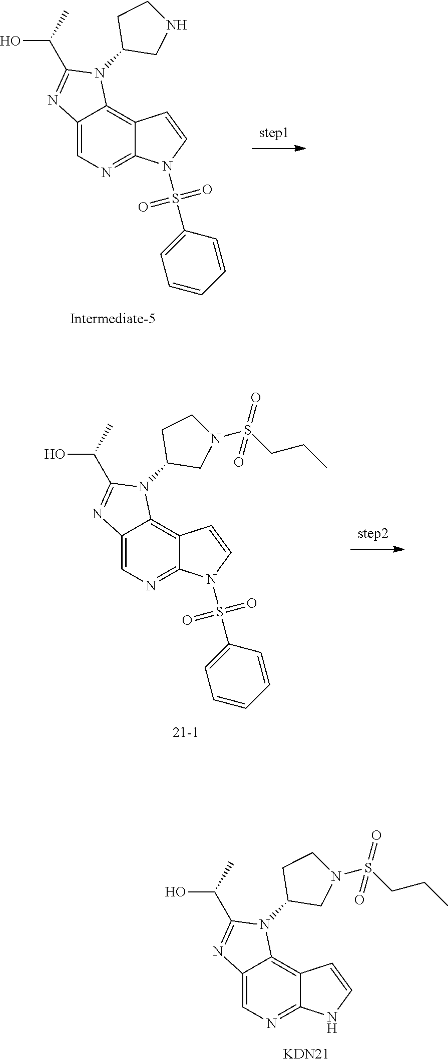

Step 1: Dissolve (R)-1-(6-(phenylsulfonyl)-1-((R-pyrrolidin-3-yl)-1,6-dihydroimidazo[4,5-d]pyrrolo[2,3-b]pyridin-2-yl)ethanol (0.3 g, 0.7 mmol) in 10 mL of tetrahydrofuran, add DIPEA (0.2 g, 1.4 mmol) and then slowly add propyl sulfonyl chloride (0.2 g, 1.4 mmol) dropwise. Raise the temperature to maintain reflux and stir for 3 h. Monitor the reaction for completion by TLC. Saturated sodium bicarbonate was then added until the reaction solution was weakly basic, and the organic phase was separated. The aqueous phase was then extracted twice using dichloromethane. The organic phases were then combined and washed with saturated saline, dried over anhydrous sodium sulfate and concentrated under vacuum to yield (R)-1-(6-(phenylsulfonyl)-1-((R)-1-(propylsulfonyl)pyrrolidin-3-yl)-1,6-dihydroimidazo[4,5-d]pyrrolo[2,3-b]pyridin-2-yl) ethanol as a yellow oil (0.3 g, 80%). The product can be used directly in the next step without further purification. HRMS (EST): m/z [M+H]⁺. C₂₃H₂₈N₅O₅S₂ calculated value 518.1526, measured value 518.1534.

Step 2: (R)-1-(6-(Phenylsulfonyl)-1-((R)-1-(propylsulfonyl)pyrrolidin-3-yl)-1,6-dihydroimidazo[4,5-d]pyrrolo[2,3-b]pyridin-2-yl) ethanol (0.3 g, 0.6 mmol) was dissolved in a solvent mixture of 5 mL tetrahydrofuran and 5 mL methanol. Then, 5 mL of 1 M sodium hydroxide was added and the mixture was then stirred for 5 h at room temperature. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS21: (R)-1-(1-((R)-1-(Propylsulfonyl)pyrrolidin-3-yl)-1,6-dihydroimidazo[4,5-d]pyrrolo[2,3-b]pyridin-2-yl)ethanol (0.2 g, 91%). ¹ HNMR (300 MHz, DMSO-d₆): δ=11.97 (s, 1H), 8.62 (s, 1H), 7.56 (s, 1H), 6.72 (s, 1H), 5.81 (d, J=6.0 Hz, 2H), 5.22 (t, J=6.0 Hz, 11H), 3.82-3.94 (m, 2H), 3.74 (t, J=9.0 Hz, 1H), 3.51-3.59 (m, 1H), 3.28 (t, J=7.5 Hz, 2H), 2.65-2.75 (m, 1H), 1.78-1.85 (m, 2H), 1.68 (d, J=6.0 Hz, 3H), 1.53 (s, 1H), 1.26 (s, 1H), 1.08 (t, J=7.5 Hz, 3H) ppm; ^{—}C NMR (75 MHz, DMSO-d₆) δ 155.12, 144.83, 136.22, 134.22, 132.43, 124.69, 104.91, 98.76, 62.52, 53.69, 49.72, 49.02, 46.86, 31.15, 22.08, 16.98, 13.32 ppm; HRMS (ESI): m/z [M+H]⁺. C₁₇H₂₄N₅O₃S calculated value 378.1594, measured value 378.1609.

### Example 22

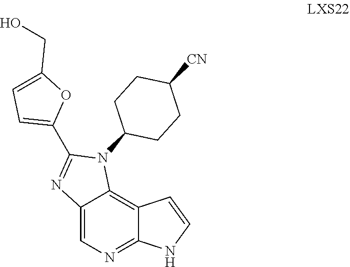 [SMILES: N#C[C@H]1CC[C@@H](n2c(-c3ccc(CO)o3)nc3cnc4[nH]ccc4c32)CC1]

### trans-4-(2-(5-(Hydroxymethyl)furan-2-yl)imidazo[4,5-d]pyrrolo[2,3-b]pyridin-1(6H)-yl)cyclohexanecarbonitrile

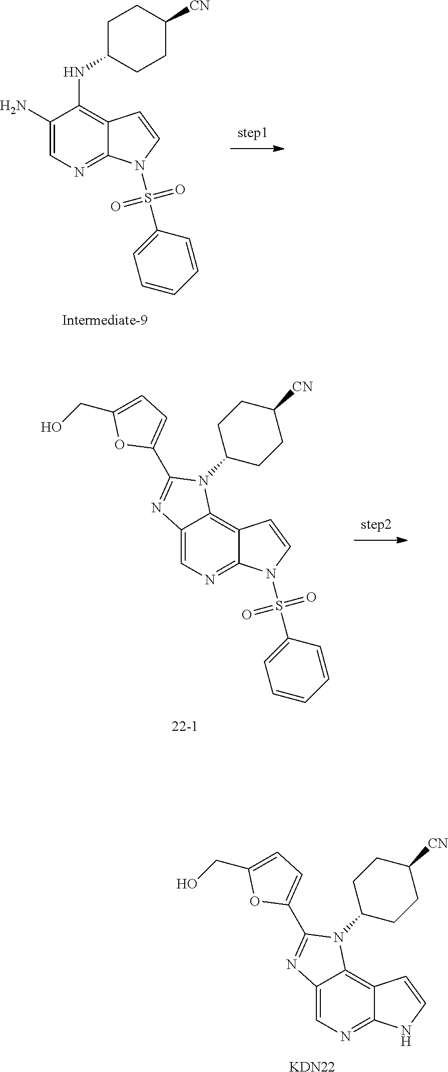

Step 1: Dissolve trans-4-((5-amino-1-(phenylsulfonyl)-1H-pyrrolo[2,3-b]pyridin-4-yl)amino)cyclohexanecarbonitrile (0.3 g, 0.8 mmol) in 10 mL of DMF, add Na₂S₂O₅ (0.8 g, 4 mmol) followed by the addition of 5-hydroxymethylfurfural (0.2 g, 1.1 mmol) dropwise. Then, raise the temperature to 90° C. and stir for 12 hours. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum. The residue was then purified using a silica gel column (petroleum ether:ethyl acetate=1:1) to yield trans-4-(2-(5-(hydroxymethyl)furan-2-yl)-6-(phenylsulfonyl)imidazo[4,5-d]pyrrolo[2,3-b]pyridin-1(6H)-yl)cyclohexanecarbonitrile as a yellow solid (0.2 g, 53%). HRMS (ESI): m/z [M+H]⁺. C₂₆H₂₄N₅O₄S calculated value 502.1544, measured value 502.1556.

Step 2: Dissolve trans-4-(2-(5-(hydroxymethyl)furan-2-yl)-6-(phenylsulfonyl)imidazo[4,5-d]pyrrolo[2,3-b]pyridin-1(6H)-yl)cyclohexanecarbonitrile (0.2 g, 0.4 mmol) in a mixture of 5 mL of tetrahydrofuran and 5 mL of methanol, add 5 mL of 1 M sodium hydroxide and stir at room temperature for 5 hours. The reaction was monitored for completion by TLC. Saturated sodium bicarbonate was then added until the reaction solution was weakly basic, and the organic phase was separated. The aqueous phase was then extracted twice using dichloromethane. The organic phases were then combined and washed with saturated saline, dried with anhydrous sodium sulfate and concentrated under vacuum, and the residue was purified using a silica gel column (petroleum ether:ethyl acetate=1:1) to yield LXS22: trans-4-(2-(5-(hydroxymethyl)furan-2-yl)imidazo[4,5-d]pyrrolo[2,3-b]pyridin-1(6H)-yl)cyclohexanecarbonitrile (0.1 g, 69%), 69%). ¹ HNMR (300 MHz, DMSO-d₆): δ=11.99 (s, 1H), 8.60 (s, 1H), 7.51 (s, 1H), 7.03 (s, 1H), 6.87 (s, 1H), 6.57 (s, 1H), 5.47 (s, 1H), 4.88 (s, 1H), 4.55 (s, 1H), 3.11 (s, 1H), 2.35 (d, J=9.0 Hz, 2H), 2.24 (d, J=12.0 Hz, 2H), 1.98 (s, 3H), 1.88 (d, J=12.0 Hz, 2H), 1.19 (t, J=9.0 Hz, 3H) ppm; ¹³ C NMR (75 MHz, DMSO-d₆) δ 153.81, 150.11, 146.67, 144.93, 142.06, 129.05, 127.92, 122.75, 120.71, 115.65, 105.93, 104.75, 99.68, 64.56, 57.69, 30.22, 26.91, 24.60 ppm: HRMS (ESI): m/z [M+H]⁺. C₂₀H₂₀N₅O₂ calculated value 362.1612, measured value 362.1625.

### Example 23

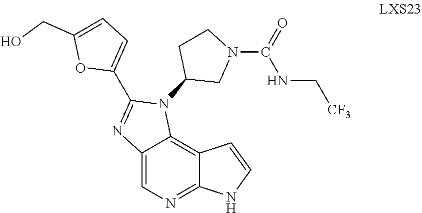 [SMILES: O=C(NCC(F)(F)F)N1CC[C@H](n2c(-c3ccc(CO)o3)nc3cnc4[nH]ccc4c32)C1]

### (R)-3-(2-(5-(Hydroxymethyl)furan-2-yl)imidazo[4,5-d]pyrrolo[2,3-b]pyridin-1(6H)-yl)-N-(2,2,2-trifluoroethyl)pyrrolidine-1-carboxamide

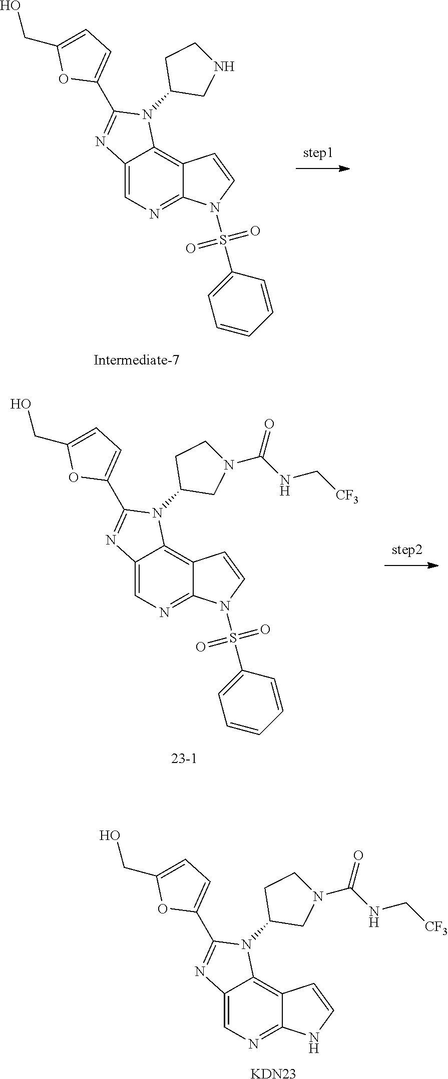

Step 1: Dissolve (R)-(5-(6-(phenylsulfonyl)-1-(pyrrolidin-3-yl)-1,6-dihydroimidazo[4,5-d]pyrrolo[2,3-b]pyridin-2-yl) furan-2-yl methanol (0.3 g, 0.6 mmol) in 10 mL of tetrahydrofuran, add carbonyl diimidazole (0.2 g, 1.2 mmol) and then slowly add trifluoromethyl ethylamine (0.2 g, 1.2 mmol) dropwise. Then, stir the mixture at room temperature for 3 h. Monitor the reaction for completion by TLC. Saturated sodium bicarbonate was then added until the reaction solution was weakly basic, and the organic phase was separated. The aqueous phase was then extracted twice using dichloromethane. The organic phases were then combined and washed with saturated saline, dried over anhydrous sodium sulfate and concentrated under vacuum to yield (R)-3-(2-(5-(hydroxymethyl)furan-2-yl)-6-(phenylsulfonyl)imidazo[4,5-d]pyrrolo[2,3-b]pyridin-1(6H)-yl)-N-(2,2,2-trifluoroethyl)pyrrolidine-1-carboxamide as a pale yellow oil (0.2 g (52%). The product can be used directly in the next step without further purification. HRMS (ESI): m/z [M+H]⁺. C₂₆H₂₄F₃N₆O₅S calculated value 589.1475, measured value 589.1482.

Step 2: (R)-3-(2-(5-(Hydroxymethyl)furan-2-yl)-6-(phenylsulfonyl)imidazo[4,5-d]pyrrolo[2,3-b]pyridin-1(6H)-yl)-N-(2,2,2-trifluoroethyl)pyrrolidine-1-carboxamide (0.2 g, 0.3 mmol) was dissolved in a solvent mixture of 5 mL tetrahydrofuran and 5 mL methanol. After adding 5 mL of 1M sodium hydroxide, the mixture was stirred at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate =1:1) to yield LXS23: (R)-3-(2-(5-(hydroxymethyl)furan-2-yl)imidazo[4,5-d]pyrrolo[2,3-b]pyridin-1(6H)-yl)-N-(2,2,2-trifluoroethyl)pyrrolidine-1-carboxamide (0.1 g, 66%). ¹ HNMR (300 MHz, DMSO-d₆): δ=12.08 (s, 1H), 8.66 (s, 1H), 7.57 (s, 1H), 7.19 (s, 1H), 7.09 (s, 1H), 6.61 (s, 1H), 6.47 (s, 1H), 5.94 (d, J=9.0 Hz, 1H), 5.49 (s, 1H), 4.57 (d, J=6.0 Hz, 2H), 3.95 (d, J=9.0 Hz, 6H), 2.71 (t, J=12.0 Hz, 1H), 1.26 (s, 2H) ppm; ¹³ C NMR (75 MHz, DMSO-d₆) δ 155.75, 153.18, 151.71, 148.07, 144.94, 142.96, 129.54, 127.72, 124.74, 120.77, 115.35, 107.53, 104.35, 99.68, 57.79, 54.88, 53.59, 48.73, 41.81, 26.20 ppm, HRMS (ESI): m/z [M+H]⁺. C₂₀H₂₀F₃N₆O₃ calculated value 449.1543, measured value 449.1550.

### Example 24

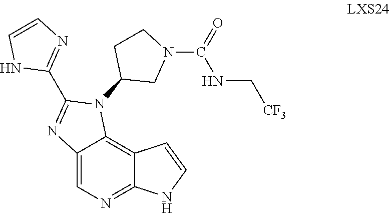 [SMILES: O=C(NCC(F)(F)F)N1CC[C@H](n2c(-c3ncc[nH]3)nc3cnc4[nH]ccc4c32)C1]

### (R)-3-(2-(1H-imidazol-2-yl)imidazo[4,5-d]pyrrolo[2,3-b]pyridin-1(6H)-yl)-N-(2,2,2-trifluoroethyl)pyrrolidine-1-carboxamide

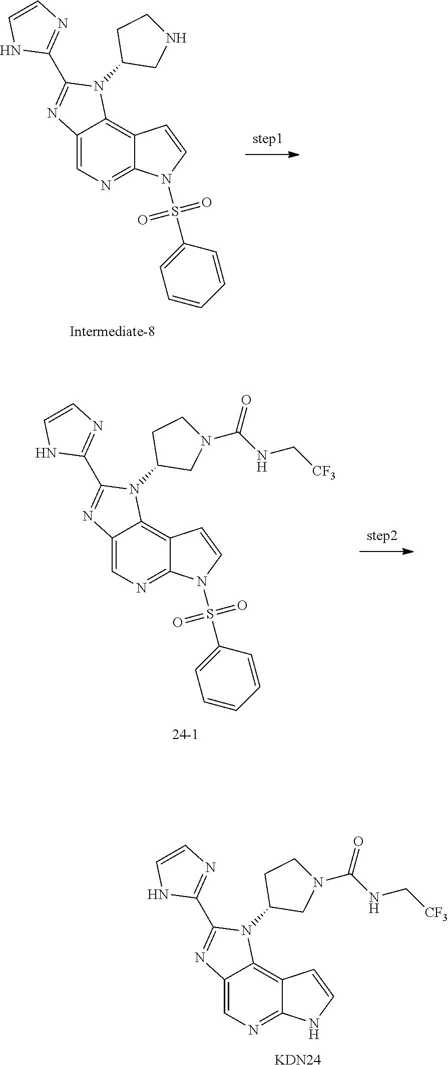

Step 1: Dissolve (R)-2-(1H-imidazol-2-yl)-6-(phenylsulfonyl)-1-(pyrrolidin-3-yl)-1,6-dihydroimidazo[4,5-d]pyrrolo[2,3-b]pyridine (0.3 g, 0.7 mmol) in 10 mL of tetrahydrofuran, add carbonyl diimidazole (0.2 g, 1.4 mmol) and then slowly add trifluoromethyl ethylamine (0.2 g, 1.4 mmol) dropwise. Then, stir the mixture at room temperature for 3 h. Monitor the reaction for completion by TLC. Saturated sodium bicarbonate was then added until the reaction solution was weakly basic, and the organic phase was separated. The aqueous phase was then extracted twice using dichloromethane. The organic phases were then combined and washed with saturated saline, dried over anhydrous sodium sulfate and concentrated under vacuum to yield (R)-3-(2-(1H-imidazol-2-yl)-6-(phenylsulfonyl)imidazo[4,5-d]pyrrolo[2,3-b]pyridin-1(6H)-yl)-N(2,2,2-trifluoroethyl)pyrrolidine-1-carboxamide as a pale yellow oil (0.2 g, 52%).). The product can be used directly in the next step without further purification. HRMS (ESI): m/z [M+H]⁺. C₂₄H₂₂F₃N₅O₃S calculated value 559.1482, measured value 559.1479.

Step 2: Dissolve (R)-3-(2-(1H-imidazol-2-yl)-6-(phenylsulfonyl)imidazo[4,5-d]pyrrolo[2,3-b]pyridin-1(6H)-yl)-N-(2,2,2-trifluoroethyl)pyrrolidine-1-carboxamide (0.2 g, 0.4 mmol) in a solvent mixture of 5 mL tetrahydrofuran and 5 mL methanol. Then, add 5 ml of 1 M of sodium hydroxide and stir the mixture at room temperature for 5 h. The reaction was monitored for completion by TLC. Saturated sodium bicarbonate was then added until the reaction solution was weakly basic, and the organic phase was separated. The aqueous phase was then extracted twice using dichloromethane. The organic phases were then combined and washed with saturated saline, dried over anhydrous sodium sulfate and concentrated under vacuum, and the residue was purified using a silica gel column (petroleum ether:ethyl acetate =1:1) to yield LXS24: (R)-3-(2-(1H-imidazol-2-yl)imidazo[4,5-d]pyrrolo[2,3-b]pyridin-1(6H)-yl)-N-(2,2,2-trifluoroethyl) pyrrolidine-1-carboxamide (0.1 g, 67%). ¹ HNMR (300 MHz, DMSO-d₆): δ=13.34 (s, 1H), 12.10 (s, 1H), 8.72 (s, 1H), 7.58 (s, 1H), 7.41 (s, 1H), 7.25 (s, 2H), 7.10 (s, 1H), 6.47 (s, 1H), 3.89-4.00 (m, 6H), 2.77 (t, J=12.0 Hz, 1H), 2.12 (s, 2H) ppm; ¹³ C NMR (75 MHz, DMSO-d₆) δ 157.08, 155.78, 148.66, 142.49, 138.11, 129.00, 127.19, 124.27, 122.77, 120.75, 115.68, 99.33, 57.26, 54.17, 48.70, 41.80, 26.63 ppm: HRMS (ESI): m/z [M+H]⁺. C₁₈H₁₈F₃N₈O calculated value 419.1550, measured value 419.1551.

### Example 25

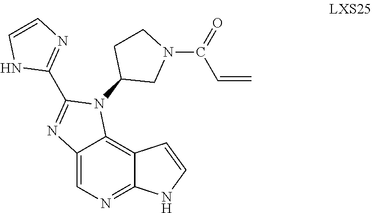 [SMILES: C=CC(=O)N1CC[C@H](n2c(-c3ncc[nH]3)nc3cnc4[nH]ccc4c32)C1]

### (R)-1-(3-(2-(1H-imidazol-2-yl)imidazo[4,5-d]pyrrolo[2,3-b]pyridin-1(6H)-yl)pyrrolidin-1-yl)prop-2-en-1-one

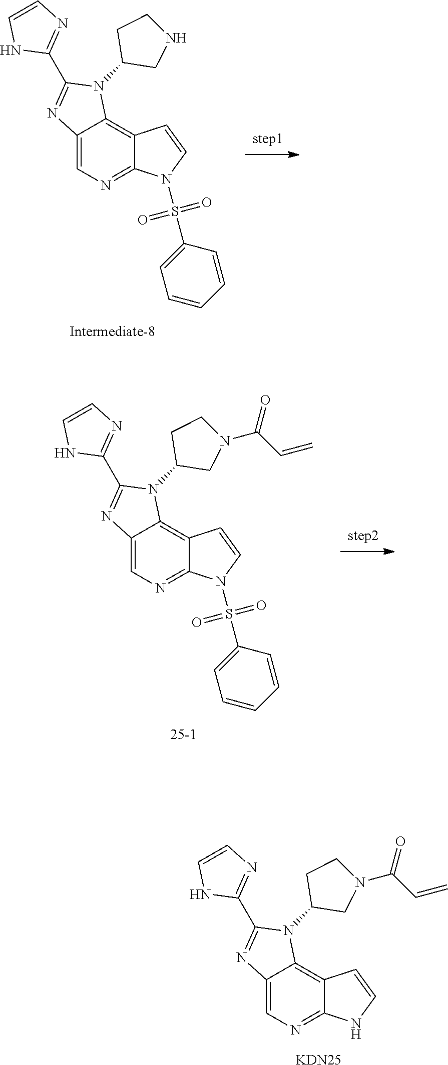

Step 1 Dissolve (R)-2-(1H-imidazol-2-yl)-6-(phenylsulfonyl)-1-(pyrrolidin-3-yl)-1,6-dihydroimidazo[4,5-d]pyrrolo[2,3-b]pyridine (0.3 g, 0.7 mmol) in 10 mL of tetrahydrofuran, add DIPEA (0.2 g, 1.4 mmol) followed by the slow dropwise addition of acryloyl chloride (0.1 g, 1.4 mmol). Then, stir the mixture at room temperature for 3 h and monitor the reaction for completion by TLC. Saturated sodium bicarbonate was then added until the reaction solution was weakly basic, and the organic phase was separated. The aqueous phase was then extracted twice using dichloromethane. The organic phases were then combined and washed with saturated saline, dried over anhydrous sodium sulfate and concentrated under vacuum to yield (R)-1-(3-(2-(1H-imidazol-2-yl)-6-(phenylsulfonyl)imidazo[4,5-d]pyrrolo[2,3-b]pyrrolidin-1(6H)-yl)pyrrolidin-1-yl)prop-2-en-1-one as a pale yellow oil (0.2 g, 59%). The product can be used directly in the next step without further purification. HRMS (ESI): m/z [M+H]⁺. C₂₄H₂₂N₇O₃S calculated value 488.1499, measured value 488.1511.

Step 2: (R)-1-(3-(2-(1H-imidazol-2-yl)-6-(phenylsulfonyl)imidazo[4,5-d]pyrrolo[2,3-b]pyridin-1(6H)-yl)pyrrolidin-1-yl)prop-2-en-1-one (0.2 g, 0.4 mmol) was dissolved in a mixture of 5 mL of tetrahydrofuran and 5 mL of methanol. Then, 5 ml of 1 M sodium hydroxide was added and the mixture was stirred at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate=1:1) to yield LXS25: (R)-1-(3-(2-(1H-imidazol-2-yl)imidazo[4,5-d]pyrrolo[2,3-b]pyridin-1(6H)-yl)pyrrolidin-1-yl)prop-2-en-1-one (0.1 g, 70%). ¹ HNMR (300 MHz, DMSO-d₆): δ=13.34 (s, 1H), 12.10 (s, 1H), 8.72 (s, 1H), 7.58 (d, J=9.0 Hz, 1H), 7.41 (s, 1H), 7.25 (s, 1H), 6.46 (s, 1H), 4.02-4.22 (m, 2H), 3.95 (t, J=9.0 Hz, 1H), 3.67 (d, J=6.0 Hz, 2H), 3.61 (d, J=6.0 Hz, 1H), 3.34 (s, 2H), 3.23 (s, 1H), 2.74-2.88 (m, 1H) ppm; ¹³ C NMR (75 MHz, DMSO-d₆) δ 166.30, 157.05, 148.66, 142.96, 138.17, 131.16, 129.05, 127.17, 126.88, 122.75, 120.74, 115.65, 99.38, 57.73, 52.07, 46.68, 27.17 ppm; HRMS (ESI): m/z [M+H]⁺. C₁₈H₁₈N₇O calculated value 348.1567, measured value 348.1557.

### Example 26

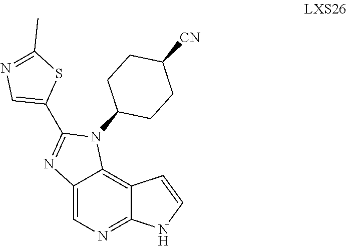 [SMILES: Cc1ncc(-c2nc3cnc4[nH]ccc4c3n2[C@H]2CC[C@@H](C#N)CC2)s1]

### trans-4-(2-(2-Methylthiazol-5-yl)imidazo[4,5-d]pyrrolo[2,3-b]pyridin-1(6H)-yl)cyclohexanecarbonitrile

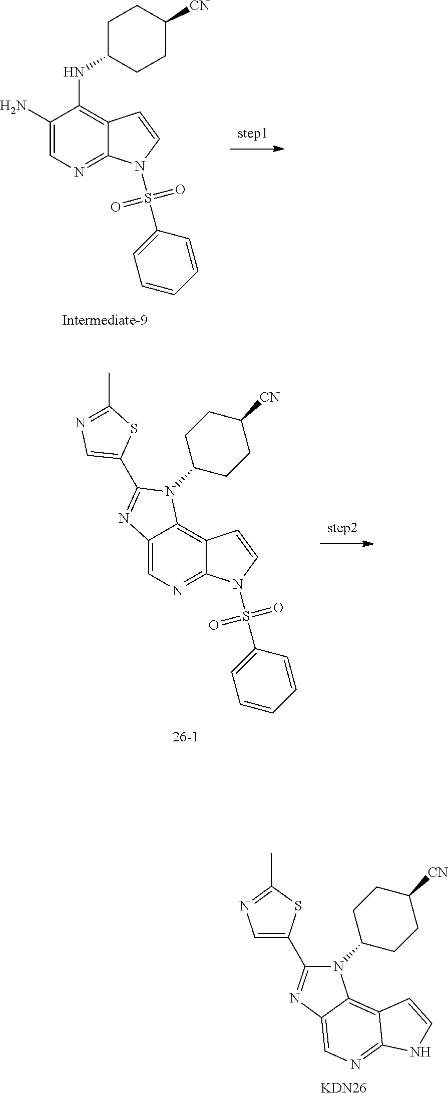

Step 1 Dissolve trans-4-((5-amino-1-(phenylsulfonyl)-1H-pyrrolo[2,3-b]pyridin-4-yl)amino)cyclohexanecarbonitrile (0.3 g, 0.8 mmol) in 10 mL of DMF, add NaS₂O₅ (0.8 g, 4 mmol) followed by the dropwise addition of 5-formyl-2-methylthiazole (0.2 g, 1.1 mmol). Stir the mixture for 12 h at 90° C. The reaction was monitored for completion by TLC. Saturated sodium bicarbonate was then added until the reaction solution was weakly basic, and the organic phase was separated. The aqueous phase was then extracted twice with dichloromethanel. The organic phases were then combined and washed with saturated saline, dried over anhydrous sodium sulfate and concentrated under vacuum to yield trans-4-(2-(2-methylthiazol-5-yl)-6-(phenylsulfonyl)imidazo[4,5-d]pyrrolo[2,3-b]pyridin-1(6H)-yl) cyclohexanecarbonitrile as a yellow oil (0.2 g, 52%). HRMS (ESI): m/z [M+H]⁺. C₂₅H₂₃N₆O₂S₂ calculated value 503.1318, measured value 503.1322.

Step 2: Trans-4-(2-(2-methylthiazol-5-yl)-6-(phenylsulfonyl)imidazo[4,5-d]pyrrolo[2,3-b]pyridin-1(6H)-yl)cyclohexanecarbonitrile (0.2 g, 0.4 mmol) was dissolved in a mixture of 5 mL of tetrahydrofuran and 5 mL of methanol, and then stirred at room temperature for 5 h after the addition of 5 mL of 1M sodium hydroxide. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS26: trans-4-(2-(2-Methylthiazol-5-yl)imidazo[4,5-d]pyrrolo[2,3-b]pyridin-1(6H)-yl)cyclohexanecarbonitrile (0.1 g, 69%).). ¹ HNMR (300 MHz, DMSO-d₆): δ=12.05 (s, 1H), 8.65 (s, 1H), 8.10 (s, 1H), 7.56 (s, 1H), 6.89 (s, 1H), 4.76 (s, 1H), 3.20 (t, J=12.0 Hz, 1H), 2.80 (s, 3H), 2.43 (t, J=12.0 Hz, 2H)=12.0 Hz, 2H), 2.27 (d, J=12.0 Hz, 2H), 2.10 (d, J=15.0 Hz, 2H), 1.86-1.98 (m, 2H) ppm; ^{—}C NMR (75 MHz, DMSO-d₆) δ 168.60, 144.99, 143.26, 143.05, 136.18, 135.29, 132.94, 127.33, 124.63, 123.32, 104.45, 100.98, 54.58, 31.18, 29.48, 29.16, 28.49, 26.19, 19.36 ppm; HRMS (ESI): m/z [M+H]⁺. C₁₉H¹⁹N₆S calculated value 363.1386, measured value 363.1395.

### Example 27

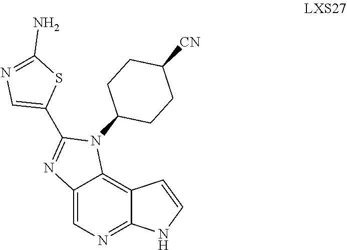 [SMILES: N#C[C@H]1CC[C@@H](n2c(-c3cnc(N)s3)nc3cnc4[nH]ccc4c32)CC1]

### trans-4-(2-(2-Aminothiazol-5-yl)imidazo[4,5-d]pyrrolo[2,3-b]pyridin-1(6H)-yl)cyclohexanecarbonitrile

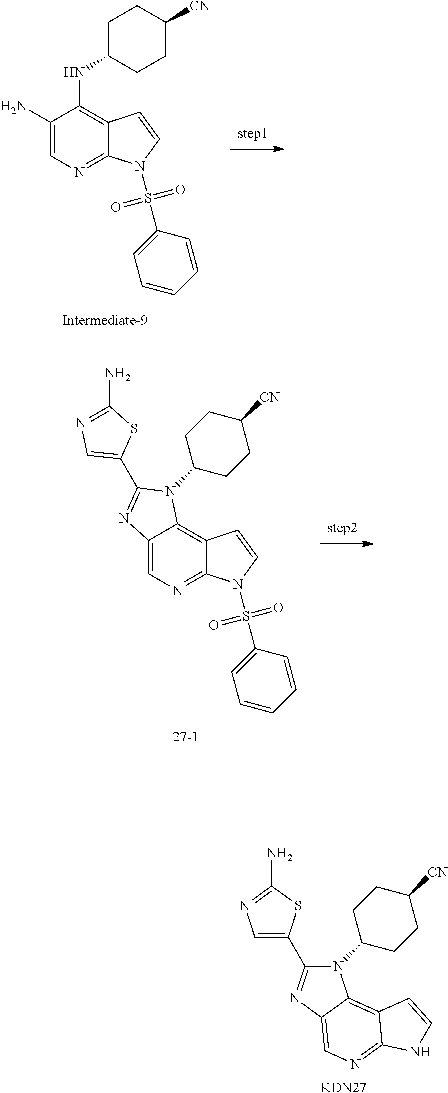

Step 1: Dissolve trans-4-((5-amino-1-(phenylsulfonyl)-1H-pyrrolo[2,3-b]pyridin-4-yl)amino)cyclohexanecarbonitrile (0.3 g, 0.8 mmol) in 10 mL of DMF, add NaS₂O₅ (0.8 g, 4 mmol) followed by the addition of 2-aminothiazole-5-carboxaldehyde (0.2 g, 1.1 mmol) dropwise. Then, stir the mixture for 12 h at 90° C. The reaction was monitored for completion by TLC. Saturated sodium bicarbonate was then added until the reaction solution was weakly basic, and the organic phase was separated. The aqueous phase was then extracted twice with dichloromethanel. The organic phases were then combined and washed with saturated saline, dried over anhydrous sodium sulfate and concentrated under vacuum to yield trans-4-(2-(2-aminothiazol-5-yl)-6-(phenylsulfonyl)imidazo[4,5-d]pyrrolo[2,3-b]pyridin-1(6H)-yl)cyclohexanecarbonitrile as a yellow oil (0.2 g, 52%). HRMS (ESI): m/z [M+H]⁺. C₂₄H₂₂N₇O₂S₂ calculated value 504.1271, measured value 504.1277.

Step 2: Trans-4-(2-(2-aminothiazol-5-yl)-6-(phenylsulfonyl)imidazo[4,5-d]pyrrolo[2,3-b]pyridin-1(6H)-yl)cyclohexanecarbonitrile (0.2 g, 0.4 mmol) was dissolved in a mixture of 5 mL of tetrahydrofuran and 5 mL of methanol, and then stirred at room temperature for 5 h after the addition of 5 mL of 1 M sodium hydroxide. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS27: trans-4-(2-(2-aminothiazol-5-yl)imidazo[4,5-d]pyrrolo[2,3-b]pyridin-1(6H)-yl)cyclohexanecarbonitrile (0.1 g, 69%).). ¹ HNMR (300 MHz, DMSO-d₆): δ=11.95 (s, 1H), 8.57 (s, 1H), 7.51 (s, 2H), 7.45 (s, 1H), 6.84 (s, 1H), 4.78 (s, 1H), 3.22 (d, J=12.0 Hz, 1H), 2.44 (t, J=13.5 Hz, 2H), 2.28 (d, J=12.0 Hz, 2H), 1.01 (d, J=12.0 Hz, 2H), 1.91 (t, J=13.5 Hz, 2H) ppm; ¹³ C NMR (75 MHz, DMSO-d₆) δ 168.94, 148.64, 144.94, 142.16, 137.74, 129.05, 127.15, 122.75, 122.29, 120.75, 115.67, 99.39, 65.97, 30.88, 26.95, 24.66 ppm; HRMS (ESI): m/z [M+H]⁺. C₁₈H₁₈N₇S calculated value 364.1339, measured value 364.1365.

### Example 28

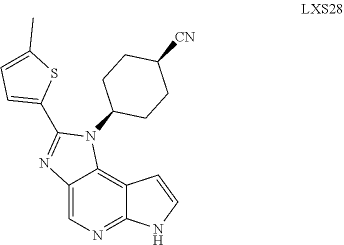 [SMILES: Cc1ccc(-c2nc3cnc4[nH]ccc4c3n2[C@H]2CC[C@@H](C#N)CC2)s1]

### trans-4-(2-(2-Methylthiophen-5-yl)imidazo[4,5-d]pyrrolo[2,3-b]pyridin-1(6H)-yl)cyclohexanecarbonitrile

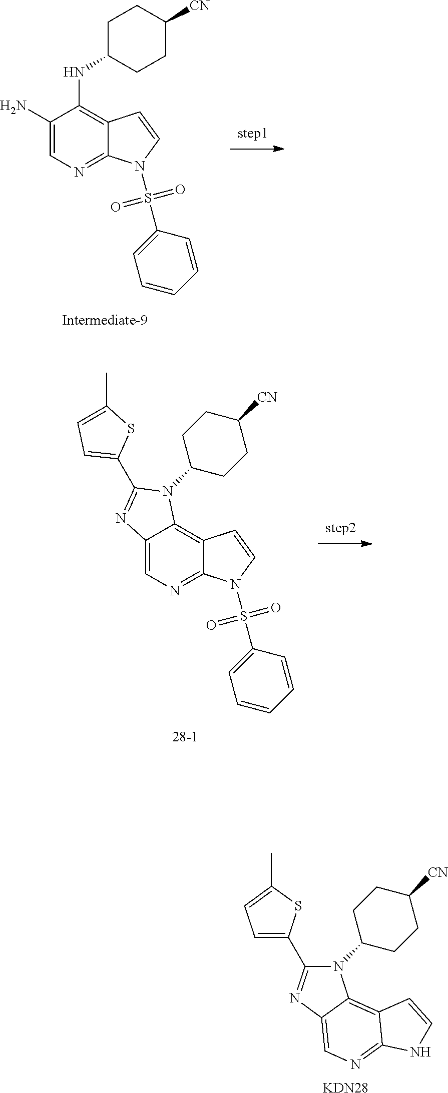

Step 1: Dissolve trans-4-((5-amino-1-(phenylsulfonyl)-1H-pyrrolo[2,3-b]pyridin-4-yl)amino)cyclohexanecarbonitrile (0.3 g, 0.8 mmol) in 10 mL of DMF, add Na₂S₂O₅ (0.8 g, 4 mmol) followed by the addition of 5-methyl-2-thiophenecarboxaldehyde (0.2 g, 1.1 mmol) dropwise, and stir for 12 h at 90° C. The reaction was monitored for completion by TLC. Saturated sodium bicarbonate was added until the reaction solution was weakly basic, and the organic phase was separated. The aqueous phase was extracted twice with dichloromethane. The organic phases were combined and washed with saturated saline, dried over anhydrous sodium sulfate and concentrated under vacuum to yield trans-4-(2-(5-methylthiophen-2-yl)-6-(phenylsulfonyl)imidazo[4,5-d]pyrrolo[2,3-b]pyridin-1(6H)-yl) cyclohexanecarbonitrile as a yellow oil (0.2 g, 53%). HRMS (ESI): m/z [M+H]⁺. C₂₆H₂₄N₅O₂S₂ calculated value 502.1366, measured value 502.1377.

Step 2: Dissolve trans-4-(2-(5-methylthiophen-2-yl)-6-(phenylsulfonyl)imidazo[4,5-d]pyrrolo[2,3-b]pyridin-1(6H)-yl) cyclohexanecarbonitrile (0.2 g, 0.4 mmol) in a mixture of 5 mL of tetrahydrofuran and 5 mL of methanol, add 5 mL of 1 M sodium hydroxide and then stir for 5 h at room temperature. The reaction was monitored for completion by TLC. Saturated sodium bicarbonate was then added until the reaction solution was weakly basic, and the organic phase was separated. The aqueous phase was then extracted twice using dichloromethane. The organic phases were then combined and washed with saturated saline, dried over anhydrous sodium sulfate and concentrated under vacuum. The residue was purified using a silica gel column (petroleum ether:ethyl acetate=1:1) to yield LXS28: trans-4-(2-(2-methylthiophen-5-yl)imidazo[4,5-d]pyrrolo[2,3-b]pyridin-1(6H)-yl)cyclohexanecarbonitrile (0.1 g, 69%).). ¹ HNMR (300 MHz, DMSO-d₆): δ=12.00 (s, 1H), 8.63 (s, 1H), 7.54 (s, 1H), 7.38 (d, J=3.0 Hz, 1H), 7.02 (s, 1H), 6.87 (s, 1H), 4.84 (d, J=12.0 Hz, 1H), 3.21 (t, J=12.0 Hz, 1H), 2.58 (s, 3H), 2.45 (t, J=12.0 Hz, 2H), 2.29 (d, J=12.0 Hz, 2H), 2.03 (d, J=6.0 Hz, 2H), 1.81-1.93 (m, 2H) ppm; ¹³ C NMR (75 MHz, DMSO-d₆) δ 145.90, 144.93, 143.26, 136.04, 135.11, 132.96, 129.68, 129.49, 127.19, 124.51, 123.28, 104.53, 100.88, 54.70, 31.17, 29.17, 28.65, 26.21, 15.41 ppm; HRMS (ESI): m/z [M+H]⁺. C₂₀H₂₀N₅S calculated value 362.1434, measured value 362.1432.

### Example 29

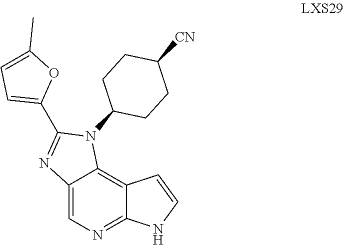 [SMILES: Cc1ccc(-c2nc3cnc4[nH]ccc4c3n2[C@H]2CC[C@@H](C#N)CC2)o1]

### trans-4-(2-(2-Methylfuran-5-yl)imidazo[4,5-d]pyrrolo[2,3-b]pyridin-1(6H)-yl)cyclohexanecarbonitrile

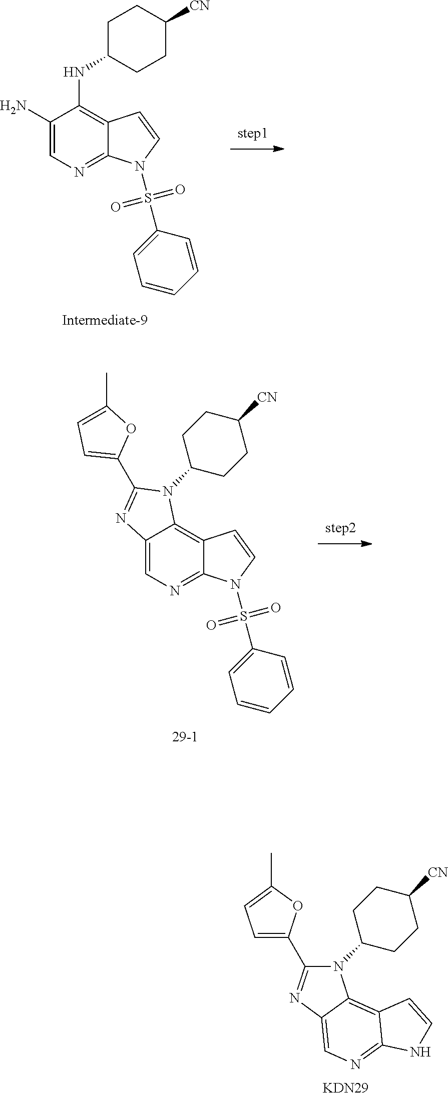

Step 1: Dissolve trans-4-((5-amino-1-(phenylsulfonyl)-1H-pyrrolo[2,3-b]pyridin-4-yl)amino)cyclohexanecarbonitrile (0.3 g, 0.8 mmol) in 10 mL of DMF, add Na₂S₂O₅ (0.8 g, 4 mmol) followed by the addition of 5-methyl-2-furanformaldehyde (0.2 g, 1.1 mmol) dropwise, and stir the mixture for 12 h at 90° C. The reaction was monitored for completion by TLC. Saturated sodium bicarbonate was then added until the reaction solution was weakly basic, and the organic phase was separated. The aqueous phase was then extracted twice with dichloromethanel and the organic phases were combined and washed with saturated saline, dried over anhydrous sodium sulfate and concentrated under vacuum to yield trans-4-(2-(5-methylfuran-2-yl)-6-(phenylsulfonyl)imidazo[4,5-d]pyrrolo[2,3-b]pyridin-1(6H)-yl) cyclohexanecarbonitrile as a yellow oil (0.2 g, 54%). HRMS (ESI): m/z [M+H]⁺. C₂₆ (H₂₄N₅O₃S calculated value 486.1594, measured value 486.1601.

Step 2: Dissolve trans-4-(2-(5-methylfuran-2-yl)-6-(phenylsulfonyl)imidazo[4,5-d]pyrrolo[2,3-b]pyridin-1(6H)-yl) cyclohexanecarbonitrile (0.2 g, 0.4 mmol) in a mixture of 5 mL of tetrahydrofuran and 5 mL of methanol, add 5 mL of 1M sodium hydroxide and stir for 5 h at room temperature. The reaction was monitored for completion by TLC. Saturated sodium bicarbonate was then added until the reaction solution was weakly basic, and the organic phase was separated. The aqueous phase was then extracted twice using dichloromethane and the organic phase were combined and washed with saturated saline, dried over anhydrous sodium sulfate and concentrated under vacuum, and the residue was purified using a silica gel column (petroleum ether:ethyl acetate=1:1) to yield LXS29: trans-4-(2-(2-Methylfuran-5-yl)imidazo[4,5-d]pyrrolo[2,3-b]pyridin-1(6H)-yl)cyclohexanecarbonitrile (0.1 g, 70%).). ¹ HNMR (300 MHz, DMSO-d₆): δ=12.00 (s, 1H), 8.62 (s, 1H), 7.53 (s, 1H), 7.01 (s, 1H), 6.89 (s, 1H), 6.41 (s, 1H), 4.89 (t, J=10.5 Hz, 1H), 3.15 (t, J=12.0 Hz, 1H), 2.53 (s, 3H), 2.40 (d, J=12.0 Hz, 2H), 2.29 (d, J=12.0 Hz, 2H), 2.00 (d, J=10.0 Hz, 2H), 1.91 (d, J=12.0 Hz, 2H) ppm; ¹³ C NMR (75 MHz, DMSO-d₆) δ 154.49, 145.07, 143.04, 142.81, 136.20, 135.14, 132.90, 124.49, 123.34, 114.47, 108.68, 104.57, 100.44, 54.96, 31.17, 29.36, 28.78, 26.41, 22.57, 13.91 ppm; HRMS (ESI): m/z [M+H]⁺. C₂₀H₂₀N₅O calculated value 346.1662, measured value 346.1664.

### Example 30

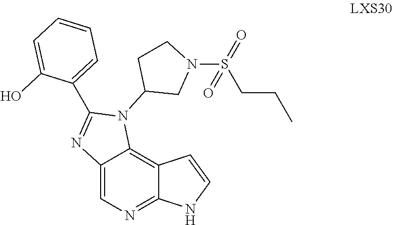 [SMILES: CCCS(=O)(=O)N1CCC(n2c(-c3ccccc3O)nc3cnc4[nH]ccc4c32)C1]

### 2-(1-(1-(propylsulfonyl)pyrrolidin-3-yl)-1,6-dihydroimidazo[4,5-d]pyrrolo[2,3-b]pyridin-2-yl)phenol

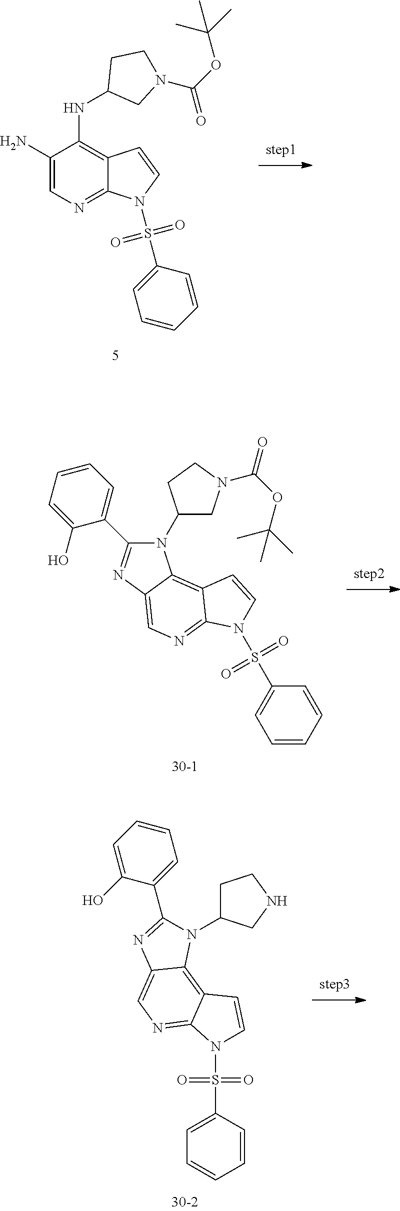 [SMILES: CC(C)(C)OC(=O)N1CCC(Nc2c(N)cnc3c2ccn3S(=O)(=O)c2ccccc2)C1 ; CC(C)(C)OC(=O)N1CCC(n2c(-c3ccccc3O)nc3cnc4c(ccn4S(=O)(=O)c4ccccc4)c32)C1 ; O=S(=O)(c1ccccc1)n1ccc2c1ncc1nc(-c3ccccc3O)n(C3CCNC3)c12]

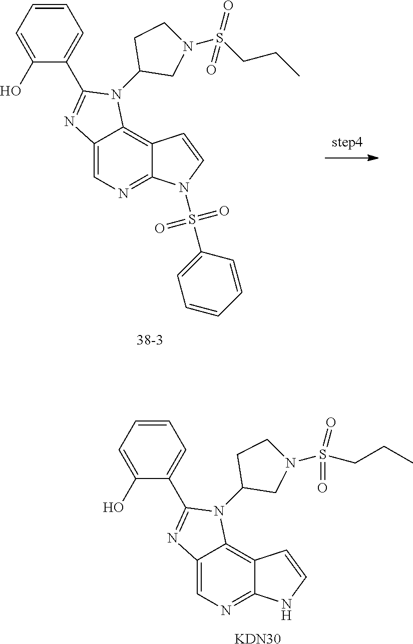 [SMILES: CCCS(=O)(=O)N1CCC(n2c(-c3ccccc3O)nc3cnc4c(ccn4S(=O)(=O)c4ccccc4)c32)C1 ; CCCS(=O)(=O)N1CCC(n2c(-c3ccccc3O)nc3cnc4[nH]ccc4c32)C1 ; "KDN30"]

Step 1: Dissolve tert-butyl 3-(((5-amino-1-(phenylsulfonyl)-1H-pyrrolo[2,3-b]pyridin-4-yl)amino)pyrrolidine-1-carboxylate (0.3 g, 0.7 mmol) in 10 mL of DMF, add Na₂S₂O₅ (0.7 g, 3.5 mmol) followed by addition of o-hydroxybenzaldehyde (0.2 g, 1.4 mmol). Stir for 12 h at 90° C. The reaction was monitored for completion by TLC. Saturated sodium bicarbonate was then added until the reaction solution was weakly basic, and the organic phase was separated. The aqueous phase was then extracted twice using dichloromethane and the organic phases were combined and washed with saturated saline, dried over anhydrous sodium sulfate and concentrated under vacuum. The residue was purified using a silica gel column (petroleum ether:ethyl acetate=1:1) to yield tert-butyl 3-(2-(2-hydroxyphenyl)-6-(phenylsulfonyl)imidazo[4,5-d]pyrrolo[2,3-b]pyridin-1(6H)-yl)pyrrolidine-1-carboxylate as a yellow solid (0.2 g, 55%). HRMS (ESI): m/z [M+H]⁺. C₂₉H₃₀N₅O₅S calculated value 560.1962, measured value 560.1965.

Step 2: Dissolve tert-butyl 3-(2-(2-hydroxyphenyl)-6-(phenylsulfonyl)imidazo[4,5-d]pyrrolo[2,3-b]pyridin-1(6H)-yl)pyrrolidine-1-carboxylate (0.2 g, 0.4 mmol) in 10 mL dichloromethane, slowly add trifluoroacetic acid (0.5 g, 4.0 mmol) and stir at room temperature for 12 h. The product was concentrated under vacuum to yield 2-(6-(phenylsulfonyl)-1-(pyrrolidin-3-yl)-1,6-dihydroimidazo[4,5-d]pyrrolo[2,3-b]pyridin-2-yl)phenol as a light yellow oil (0.1 g, 61%). The product can be used directly in the next step without further purification. HRMS (ESI): m/z [M+H]⁺. C₂₄H₂₂N₅O₃S calculated value 460.1438, measured value 460.1441.

Step 3: Dissolve 2-(6-(phenylsulfonyl)-1-(pyrrolidin-3-yl)-1,6-dihydroimidazo[4,5-d]pyrrolo[2,3-b]pyridin-2-yl)phenol (0.1 g, 0.2 mmol) in 10 mL of tetrahydrofuran, add DIPEA (0.1 g, 0.4 mmol) followed by the slow dropwise addition of propylsulfonyl chloride (0.1 g, 0.3 mmol) and stir for 3 h at room temperature. The reaction was monitored for completion by TLC. Saturated sodium bicarbonate was then added until the reaction solution was weakly basic, and the organic phase was separated. The aqueous phase was then extracted twice using dichloromethane and the organic phases were combined and washed with saturated saline, dried over anhydrous sodium sulfate and concentrated under vacuum to yield 2-(6-(phenylsulfonyl)-1-(1-(propylsulfonyl)pyrrolidin-3-yl)-1,6-dihydroimidazo[4,5-d]pyrrolo[2,3-b]pyridin-2-yl)phenol as a pale yellow oil (0.1 g, 81%). The product can be used directly in the next step without further purification. HRMS (ESI): m/z [M+H]⁺. C₂₇H₂₈N₅O₅S₂ calculated value 566.1526, measured value 566.1533.

Step 4: Dissolve 2-(6-(phenylsulfonyl)-1-(1-(propylsulfonyl)pyrrolidin-3-yl)-1,6-dihydroimidazo[4,5-d]pyrrolo[2,3-b]pyridin-2-yl)phenol (0.1 g, 0.2 mmol) in a mixture of 5 mL of tetrahydrofuran and 5 mL of methanol. Then, add 5 mL of 1 M sodium hydroxide and then stir the mixture at room temperature for 5 hours. The reaction was monitored for completion by TLC. Saturated sodium bicarbonate was then added until the reaction solution was weakly basic, and the organic phase was separated. The aqueous phase was then extracted twice using dichloromethane and the organic phases were combined and washed with saturated saline, dried with anhydrous sodium sulfate and concentrated under vacuum, and the residue was purified using a silica gel column (petroleum ether:ethyl acetate=1:1) to yield LXS30: 2-(1-(1-(propylsulfonyl)pyrrolidin-3-yl)-1,6-dihydroimidazo[4,5-d]pyrrolo[2,3-b]pyridin-2-yl)phenol (65.0 mg, 86%). ¹ HNMR (300 MHz, DMSO-d₆): δ=12.06 (s, 1H), 8.67 (s, 1H), 7.59 (t, J=3.0 Hz, 1H), 7.49-7.52 (m, 1H), 7.41-7.47 (m, 1H), 7.19 (d, J=9.0 Hz, 1H), 7.01 (t, J=7.5 Hz, 1H), 6.80 (d, J=3.0 Hz, 1H), 5.12-5.24 (m, 1H), 3.98-4.10 (m, 1H), 3.74-3.87 (m, 2H), 3.19-3.25 (m, 2H), 2.69-2.83 (m, 1H), 2.33-2.42 (m, 1H), 1.77-1.84 (m, 2H), 1.05 (t, J=9.0 Hz, 3H) ppm; ¹³ C NMR (75 MHz, DMSO-d₆) δ 170.82, 156.82, 150.94, 144.75, 136.04, 135.50, 132.54, 131.85, 131.74, 124.82, 119.58, 117.92, 116.59, 104.91, 98.80, 60.22, 55.10, 49.24, 46.75, 30.10, 21.24, 16.91, 14.55, 13.32 ppm; HRMS (ESI): m/z [M+H]⁺. C₂₁H₂₄N₅O₃S calculated value 426.1594, Measured value 426.1462.

### Example 31

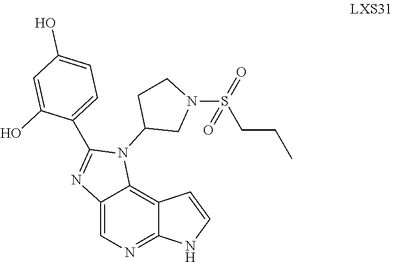 [SMILES: CCCS(=O)(=O)N1CCC(n2c(-c3ccc(O)cc3O)nc3cnc4[nH]ccc4c32)C1]

### 4-(1-(1-(Propylsulfonyl)pyrrolidin-3-yl)-1,6-dihydroimidazo[4,5-d]pyrrolo[2,3-b]pyridin-2-yl)benzene-1,3-diol

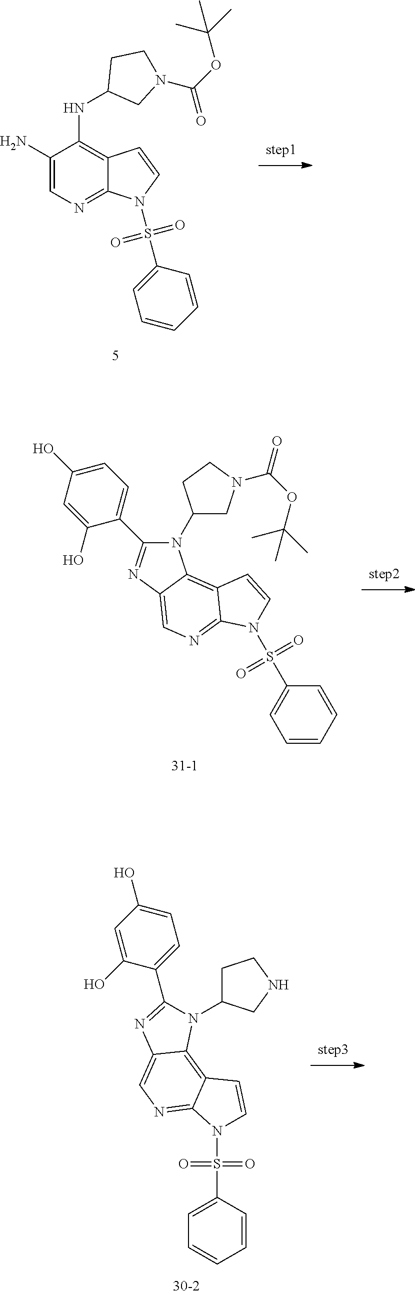 [SMILES: CC(C)(C)OC(=O)N1CCC(Nc2c(N)cnc3c2ccn3S(=O)(=O)c2ccccc2)C1 ; CC(C)(C)OC(=O)N1CCC(n2c(-c3ccc(O)cc3O)nc3cnc4c(ccn4S(=O)(=O)c4ccccc4)c32)C1 ; O=S(=O)(c1ccccc1)n1ccc2c1ncc1nc(-c3ccc(O)cc3O)n(C3CCNC3)c12]

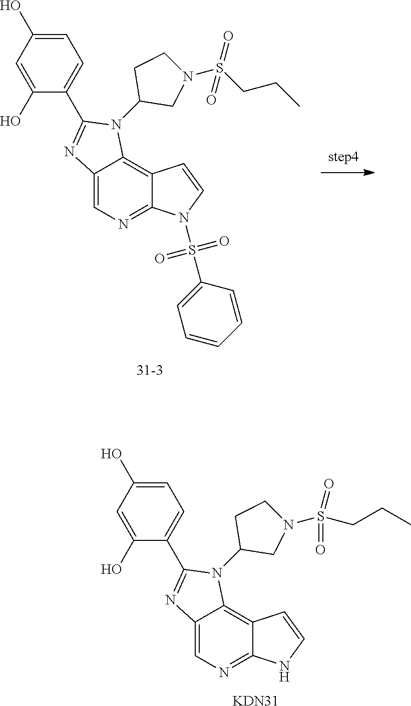 [SMILES: CCCS(=O)(=O)N1CCC(n2c(-c3ccc(O)cc3O)nc3cnc4c(ccn4S(=O)(=O)c4ccccc4)c32)C1 ; CCCS(=O)(=O)N1CCC(n2c(-c3ccc(O)cc3O)nc3cnc4[nH]ccc4c32)C1 ; "KDN31"]

Step 1: Dissolve tert-butyl 3-(((5-amino-1-(phenylsulfonyl)-1H-pyrrolo[2,3-b]pyridin-4-yl)amino)pyrrolidine-1-carboxylate (0.3 g, 0.7 mmol) in 10 mL of DMF, add Na₂S₂O₅ (0.7 g, 3.5 mmol) followed by addition of 2,4-dihydroxybenzaldehyde (0.2 g, 1.4 mmol). Stir for 12 h at 90° C. The reaction was monitored for completion by TLC. Saturated sodium bicarbonate was then added until the reaction solution was weakly basic, and the organic phase was separated. The aqueous phase was then extracted twice using dichloromethane and the organic phases were combined and washed with saturated saline, dried over anhydrous sodium sulfate and concentrated under vacuum. The residue was purified using a silica gel column (petroleum ether:ethyl acetate=1:1) to yield tert-butyl 3-(2-(2,4-dihydroxyphenyl)-6-(phenylsulfonyl)imidazo[4,5-d]pyrrolo[2,3-b]pyridin-1(6H)-yl)pyrrolidine-1-carboxylate as a yellow solid (0.3 g, 79%). HRMS (ESI): m/z [M+H]⁺. C₂₉H₃₀N₅O₆S calculated value 576.1911, measured value 576.1919.

Step 2: Dissolve tert-butyl 3-(2-(2,4-dihydroxyphenyl)-6-(phenylsulfonyl)imidazo[4,5-d]pyrrolo[2,3-b]pyridin-1(6H)-yl)pyrrolidine-1-carboxylate (0.3 g, 0.5 mmol) in 10 mL dichloromethane, slowly add trifluoroacetic acid (0.6 g, 5.0 mmol) and stir at room temperature for 12 hours. The product is then concentrated under vacuum to yield 4-(6-(phenylsulfonyl)-1-(pyrrolidin-3-yl)-1,6-dihydroimidazo[4,5-d]pyrrolo[2,3-b]pyridin-2-yl)benzene-1,3-diol as a light yellow oil (0.2 g, 81%). The product can be used directly in the next step without further purification. HRMS (ESI): m/z [M+H]. C₂₄H₂₂N₅O₄S calculated value 476.1387, measured value 476.1391.

Step 3: Dissolve 4-(6-(phenylsulfonyl)-1-(pyrrolidin-3-yl)-1,6-dihydroimidazo[4,5-d]pyrrolo[2,3-b]pyridin-2-yl)benzene-1,3-diol (0.2 g, 0.4 mmol) in 10 mL of tetrahydrofuran, add DIPEA (0.1 g, 0.8 mmol) followed by the slow dropwise addition of propylsulfonyl chloride (0.1 g, 0.6 mmol). Stir at room temperature for 3 h. The reaction was monitored by TLC for completion. Saturated sodium bicarbonate was then added until the reaction solution was weakly basic, and the organic phase was separated. The aqueous phase was then extracted twice using dichloromethane and the organic phases were combined and washed with saturated saline, dried over anhydrous sodium sulfate and concentrated under vacuum to yield 4-(6-III (phenylsulfonyl)-1-(1-(propylsulfonyl)pyrrolidin-3-yl)-1,6-dihydroimidazo[4,5-d]pyrrolo[2,3-b]pyridin-2-yl) benzene-1,3-diol as a pale yellow oil (0.2 g, 82%). The product can be used directly in the next step without further purification. HRMS (ESI): m/z [M+H]⁺. C₂₇H₂₈N₅O₆S₂ calculated value 582.1476, measured value 582.1501.

Step 4: Dissolve 4-(6-(phenylsulfonyl)-1-(1-(propylsulfonyl)pyrrolidin-3-yl)-1,6-dihydroimidazo[4,5-d]pyrrolo[2,3-b]pyridin-2-yl) benzene-1,3-diol (0.2 g, 0.3 mmol) in a solvent mixture of 5 mL tetrahydrofuran and 5 mL methanol, after adding 5 mL of 1 M sodium hydroxide, stir the mixture for 5 h at room temperature and monitor the reaction for completion by TLC. Saturated sodium bicarbonate was then added until the reaction solution was weakly basic, and the organic phase was separated. The aqueous phase was then extracted twice using dichloromethane and the organic phases were combined and washed with saturated saline, dried over anhydrous sodium sulfate and concentrated under vacuum, and the residue was purified using a silica gel column (petroleum ether:ethyl acetate=1:1) to yield LXS31: 4-(1-(1-(propylsulfonyl)pyrrolidin-3-yl)-1,6-dihydroimidazo[4,5-d]pyrrolo[2,3-b]pyridin-2-yl)benzene-1,3-diol (0.1 g, 66%). ¹ HNMR (300 MHz, DMSO-d₆): δ=11.97 (s, 1H), 10.15 (s, 1H), 9.84 (s, 1H), 8.63 (s, 1H), 7.57 (s, 1H), 7.29 (d, J=9.0 Hz, 1H), 6.76 (d, J=3.0 Hz, 1H), 6.53 (d, J=3.0 Hz, 1H), 6.42-6.46 (m, 1H), 5.12-5.34 (m, 1H), 3.95 (t, J=9.0 Hz, 1H), 3.73-3.81 (m, 2H), 3.17-3.23 (m, 2H), 2.66-2.80 (m, 1H), 2.30-2.38 (m, 1H), 1.72-1.85 (m, 2H), 1.28-1.31 (m, 2H), 1.08-1.19 (m, 2H), 1.02 (t, J=9.0 Hz, 3H) ppm; ¹³ C NMR (75 MHz, DMSO-d₆) δ 160.74, 157.62, 151.30, 144.70, 135.81, 135.33, 133.33, 131.70, 124.76, 108.69, 107.72, 104.91, 102.94, 98.78, 54.98, 53.91, 49.43, 46.70, 18.47, 16.95, 13.34 ppm: HRMS (ESI): m/z [M+H]⁺. C₂₁H₂₄N₅O₄S calculated value 442.1544, measured value 442.1405.

### Example 32

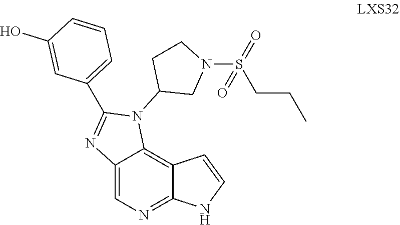 [SMILES: CCCS(=O)(=O)N1CCC(n2c(-c3cccc(O)c3)nc3cnc4[nH]ccc4c32)C1]

### 3-(1-(1-(propylsulfonyl)pyrrolidin-3-yl)-1,6-dihydroimidazo[4,5-d]pyrrolo[2,3-b]pyridin-2-yl)phenol

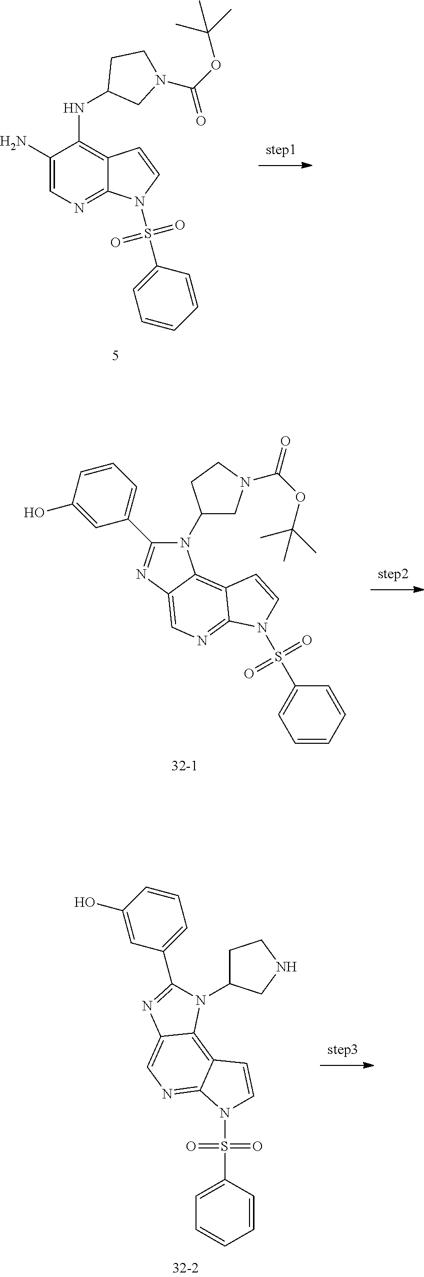 [SMILES: CC(C)(C)OC(=O)N1CCC(Nc2c(N)cnc3c2ccn3S(=O)(=O)c2ccccc2)C1 ; CC(C)(C)OC(=O)N1CCC(n2c(-c3cccc(O)c3)nc3cnc4c(ccn4S(=O)(=O)c4ccccc4)c32)C1 ; O=S(=O)(c1ccccc1)n1ccc2c1ncc1nc(-c3cccc(O)c3)n(C3CCNC3)c12]

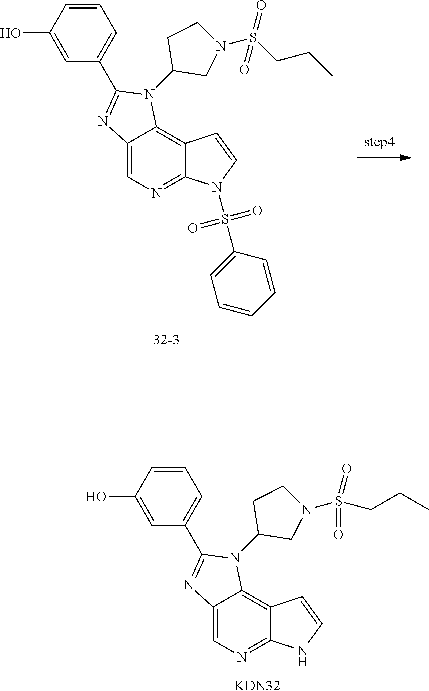 [SMILES: CCCS(=O)(=O)N1CCC(n2c(-c3cccc(O)c3)nc3cnc4c(ccn4S(=O)(=O)c4ccccc4)c32)C1 ; CCCS(=O)(=O)N1CCC(n2c(-c3cccc(O)c3)nc3cnc4[nH]ccc4c32)C1 ; "KDN32"]

Step 1: Dissolve tert-butyl 3-(((5-amino-1-(phenylsulfonyl)-1H-pyrrolo[2,3-b]pyridin-4-yl)amino)pyrrolidine-1-carboxylate (0.3 g, 0.7 mmol) in 10 mL of DMF, add Na₂S₂O₅ (0.7 g, 3.5 mmol) followed by adding 3-hydroxybenzaldehyde (0.2 g, 1.4 mmol). Stir for 12 h at 90° C. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum. The residue was purified using a silica gel column (petroleum ether:ethyl acetate=1:1) to yield tert-butyl 3-(2-(3-hydroxyphenyl)-6-(phenylsulfonyl)imidazo[4,5-d]pyrrolo[2,3-b]pyridin-1(6H)-yl)pyrrolidine-1-carboxylate as a yellow solid (0.3 g, 82%). HRMS (ESI): m/z [M+H]⁺. C₂₉H₃₀N₅O₅S calculated value 560.1962, measured value 560.1969.

Step 2: Dissolve tert-butyl 3-(2-(3-hydroxyphenyl)-6-(phenylsulfonyl)imidazo[4,5-d]pyrrolo[2,3-b]pyridin-1(6H)-yl)pyrrolidine-1-carboxylate (0.3 g, 0.5 mmol) in 10 mL of dichloromethane, slowly add trifluoroacetic acid (0.6 g, 5.0 mmol) and stir at room temperature for 12 h. The product was concentrated under vacuum to yield 3-(6-(phenylsulfonyl)-1-(pyrrolidin-3-yl)-1,6-dihydroimidazo[4,5-d]pyrrolo[2,3-b]pyridin-2-yl) phenol as a light yellow oil (0.2 g, 81%). The product can be used directly in the next step without further purification. HRMS (ESI): m/z [M+H]⁺. C₂₄H₂₂N₅O₃S calculated value 460.1438, measured value 460.1441.

Step 3: Dissolve 3-(6-(phenylsulfonyl)-1-(pyrrolidin-3-yl)-1,6-dihydroimidazo[4,5-d]pyrrolo[2,3-b]pyridin-2-yl)phenol (0.2 g, 0.4 mmol) in 10 mL of tetrahydrofuran, add DIPEA (0.1 g, 0.8 mmol) followed by slow dropwise addition of propylsulfonyl chloride (0.1 g, 0.6 mmol) and then stir for 3 h at room temperature. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield 3-(6-(phenylsulfonyl)-1-(1-(propylsulfonyl)pyrrolidin-3-yl)-1,6-dihydroimidazo[4,5-d]pyrrolo[2,3-b]pyridin-2-yl) phenol as a pale yellow oil (0.2 g, 81%). The product can be used directly in the next step without further purification. HRMS (ESI): m/z [M+H]⁺. C₂₇H₂₈N₅O₅S₂ calculated value 566.1526, measured value 566.1529.

Step 4: Dissolve 3-(6-(phenylsulfonyl)-1-(1-(propylsulfonyl)pyrrolidin-3-yl)-1,6-dihydroimidazo[4,5-d]pyrrolo[2,3-b]pyridin-2-yl) phenol (0.2 g, 0.4 mmol) in a mixture of 5 mL of tetrahydrofuran and 5 mL of methanol, add 5 mL of 1M sodium hydroxide and then stir at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS32: 3-(1-(1-(propylsulfonyl)pyrrolidin-3-yl)-1,6-dihydroimidazo[4,5-d]pyrrolo[2,3-b]pyridin-2-yl)phenol (0.1 g, 66%). ¹ HNMR (300 MHz, DMSO-d₆): δ=12.06 (s, 1H), 10.04 (s, 1H), 8.68 (s, 1H), 7.60 (t, J=3.0 Hz, 1H), 7.43 (t, J=7.5 Hz, 1H), 7.20 (t, J=6.0 Hz, 1H), 7.00-7.03 (m, 1H), 6.80 (d, J=3.0 Hz, 1H), 5.41-5.53 (m, 1H), 3.99-4.09 (m, 1H), 3.76-3.89 (m, 2H), 3.46-3.55 (m, 1H), 3.16-3.32 (m, 2H), 2.71-2.85 (m, 1H), 2.40-2.49 (m, 1H), 1.72-1.84 (m, 2H), 1.05 (t, J=9.0 Hz, 3H) ppm; ¹³ C NMR (75 MHz, DMSO-d₆) δ 158.03, 152.38, 144.82, 136.27, 135.14, 132.22, 131.63, 130.44, 120.62, 117.30, 116.85, 111.64, 105.14, 55.07, 49.05, 30.03, 16.88, 13.35 ppm; HRMS (ESI): m/z [M+H]⁺. C₂₁H₂₄N₅O₃S calculated value 426.1594, measured value 426.1464.

### Example 33

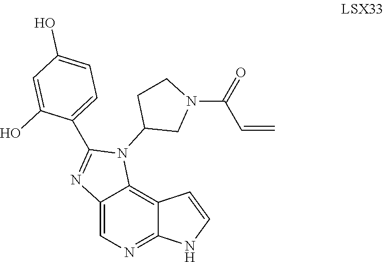 [SMILES: C=CC(=O)N1CCC(n2c(-c3ccc(O)cc3O)nc3cnc4[nH]ccc4c32)C1]

### 1-(3-(2-(2,4-Dihydroxyphenyl)imidazo[4,5-d]pyrrolo[2,3-b]pyridin-1(6H)-yl)pyrrolidin-1-yl)prop2-en-1-one

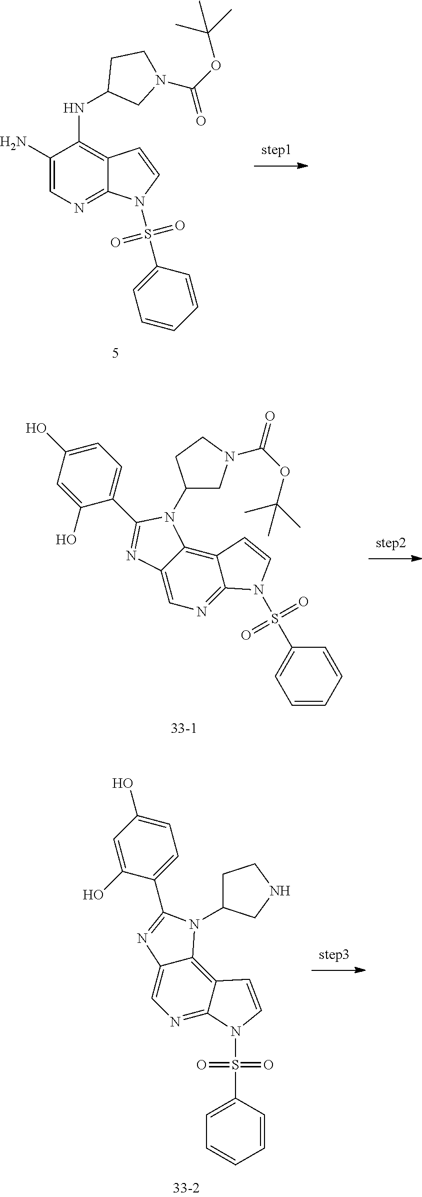 [SMILES: CC(C)(C)OC(=O)N1CCC(Nc2c(N)cnc3c2ccn3S(=O)(=O)c2ccccc2)C1 ; CC(C)(C)OC(=O)N1CCC(n2c(-c3ccc(O)cc3O)nc3cnc4c(ccn4S(=O)(=O)c4ccccc4)c32)C1 ; O=S(=O)(c1ccccc1)n1ccc2c1ncc1nc(-c3ccc(O)cc3O)n(C3CCNC3)c12]

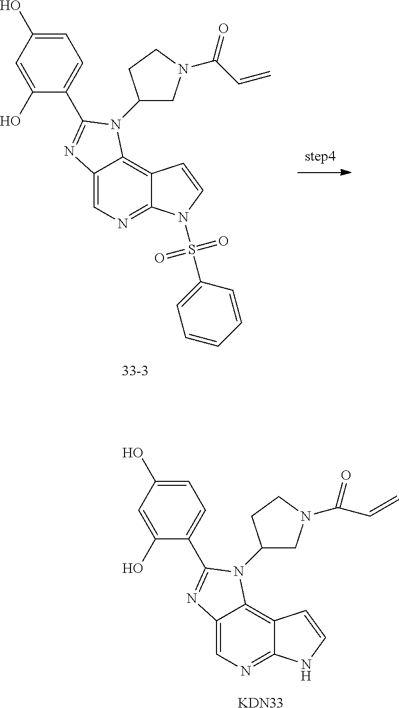

Step 1: Dissolve tert-butyl 3-(((5-amino-1-(phenylsulfonyl)-1H-pyrrolo[2,3-b]pyridin-4-yl)amino)pyrrolidine-1-carboxylate (0.3 g, 0.7 mmol) in 10 mL of DMF, add Na₂S₂O₅ (0.7 g, 3.5 mmol) followed by adding 2,4-dihydroxybenzaldehyde (0.2 g, 1.4 mmol). Stir for 12 h at 90° C. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum. The residue was purified using a silica gel column (petroleum ether:ethyl acetate=1:1) to yield tert-butyl 3-(2-(2,4-dihydroxyphenyl)-6-(phenylsulfonyl)imidazo[4,5-d]pyrrolo[2,3-b]pyridin-1(6H)-yl)pyrrolidine-1-carboxylate as a yellow solid (0.3 g, 79%). HRMS (ESI): m/z [M+H]⁺. C₂₉H₃₀N₅O₆S calculated value 576.1911, measured value 576.1918.

Step 2: Dissolve tert-butyl 3-(2-(2,4-dihydroxyphenyl)-6-(phenylsulfonyl)imidazo[4,5-d]pyrrolo[2,3-b]pyridin-1(6H)-yl)pyrrolidine-1-carboxylate (0.3 g, 0.5 mmol) in 10 mL dichloromethane, slowly add trifluoroacetic acid (0.6 g, 5.0 mmol) and stir at room temperature for 12 hours. The product was concentrated under vacuum to yield 4-(6-(phenylsulfonyl)-1-(pyrrolidin-3-yl)-1,6-dihydroimidazo[4,5-d]pyrrolo[2,3-b]pyridin-2-yl)benzene-1,3-diol as a light yellow oil (0.2 g, 81%). The product can be used directly in the next step without further purification. HRMS (EST): m/z [M+H]⁺. C₂₄H₂₂N₅O₄S calculated value 476.1387, measured value 476.1375.

Step 3: Dissolve 4-(6-(phenylsulfonyl)-1-(pyrrolidin-3-yl)-1,6-dihydroimidazo[4,5-d]pyrrolo[2,3-b]pyridin-2-yl)benzene-1,3-diol (0.2 g, 0.4 mmol) in 10 mL of tetrahydrofuran, add DIPEA (0.1 g, 0.8 mmol) followed by slow dropwise addition of acryloyl chloride (0.1 g, 0.6 mmol). Stir at room temperature for 3 h. The reaction was monitored by TLC for completion. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield 1-(3-(2-(2,4-dihydroxyphenyl)-6-(phenylsulfonyl)imidazo[4,5-d]pyrrolo[2,3-b]pyridin-1(6H)-yl)pyrrolidin-1-yl)prop-2-en-1-one as a pale yellow oil (0.2 g, 90%). The product can be used directly in the next step without further purification. HRMS (ESI): m/z [M+H]⁺. C₂₇H₂₄N₅O₅S calculated value 530.1493, measured value 530.1488.

Step 4: Dissolve 1-(3-(2-(2,4-dihydroxyphenyl)-6-(phenylsulfonyl)imidazo[4,5-d]pyrrolo[2,3-b]pyridin-1(6H)-yl)pyrrolidin-1-yl)prop-2-en-1-one (0.2 g, 0.4 mmol) in a mixture of 5 mL of tetrahydrofuran and 5 mL of methanol, add 5 mL of 1 M sodium hydroxide. Then, stir the mixture for 5 h at room temperature, the reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS33: 1-(3-(2-(2,4-Dihydroxyphenyl)imidazo[4,5-d]pyrrolo[2,3-b]pyridin-1(6H)-yl)pyrrolidin-1-yl)prop-2-en-1-one (0.1 g, 68%). ¹ HNMR (300 MHz, DMSO-d₆): δ=11.96 (s, 1H), 8.62 (s, 1H), 7.52 (d, J=6.0 Hz, 1H), 7.26 (d, J=6.0 Hz, 1H), 6.55 (s, 1H), 6.42 (d, J=9.0 Hz, 1H), 4.03-4.10 (m, 4H), 3.55-3.66 (m, 3H), 2.68-2.79 (m, 1H), 2.32 (s, 1H), 2.02 (s, 4H), 1.21 (t, J=6.0 Hz, 4H) ppm; ¹³ C NMR (75 MHz, DMSO-d₆) δ 166.39, 159.94, 156.62, 153.78, 148.64, 142.18, 131.16, 130.11, 129.05, 127.14, 126.89, 120.74, 115.69, 110.95, 109.05, 105.64, 99.38, 57.77, 52.05, 46.69, 27.17 ppm; HRMS (ESI): m/z [M+H]⁺. C₂₁H₂₀N₅O₃ calculated value 390.1561, measured value 390.1692.

### Example 34

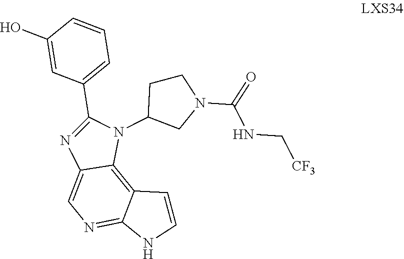 [SMILES: O=C(NCC(F)(F)F)N1CCC(n2c(-c3cccc(O)c3)nc3cnc4[nH]ccc4c32)C1]

### 3-(2-(3-Hydroxyphenyl)imidazo[4,5-d]pyrrolo[2,3-b]pyridin-1(6H)-yl)-N-(2,2,2-trifluoroethyl)pyrrolidine-1-carboxamide

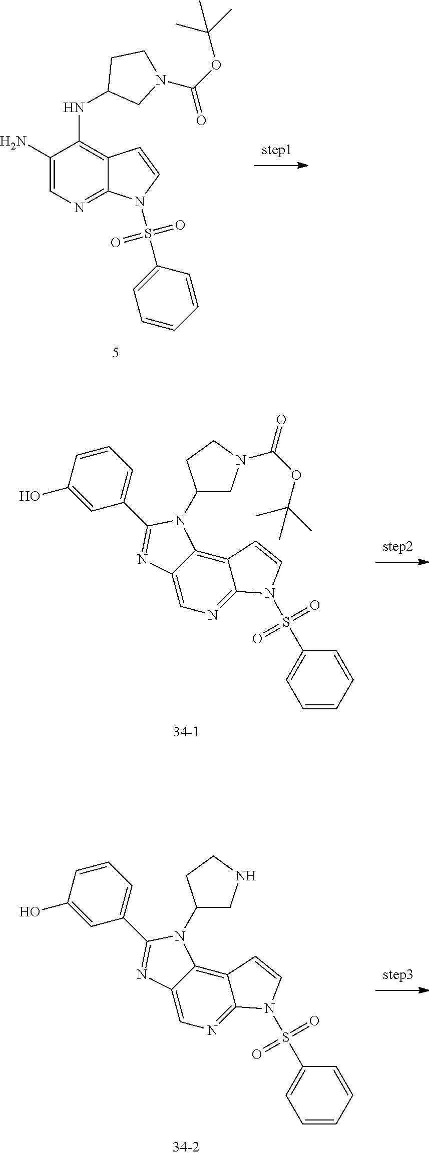 [SMILES: CC(C)(C)OC(=O)N1CCC(Nc2c(N)cnc3c2ccn3S(=O)(=O)c2ccccc2)C1 ; CC(C)(C)OC(=O)N1CCC(n2c(-c3cccc(O)c3)nc3cnc4c(ccn4S(=O)(=O)c4ccccc4)c32)C1 ; O=S(=O)(c1ccccc1)n1ccc2c1ncc1nc(-c3cccc(O)c3)n(C3CCNC3)c12]

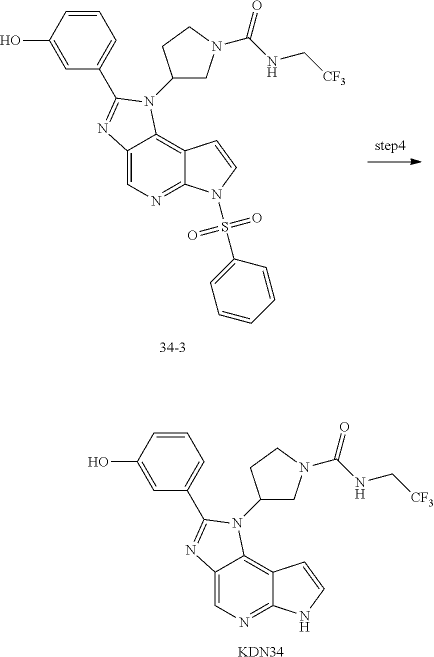

Step 1: Dissolve tert-butyl 3-(((5-amino-1-(phenylsulfonyl)-1H-pyrrolo[2,3-b]pyridin-4-yl)amino)pyrrolidine-1-carboxylate (0.3 g, 0.7 mmol) in 10 mL of DMF, add Na₂S₂O₅ (0.7 g, 3.5 mmol) followed by adding 3-hydroxybenzaldehyde (0.2 g, 1.4 mmol). Stir for 12 h at 90° C. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum. The residue was purified using a silica gel column (petroleum ether:ethyl acetate=1:1) to yield tert-butyl 3-(2-(3-hydroxyphenyl)-6-(phenylsulfonyl)imidazo[4,5-d]pyrrolo[2,3-b]pyridin-1(6H)-yl)pyrrolidine-1-carboxylate as a yellow solid (0.3 g, 82%). HRMS (ESI): m/z [M+H]⁺. C₂₉H₃₀N₅O₅S calculated value 560.1962, measured value 560.1977.

Step 2: Dissolve tert-butyl 3-(2-(3-hydroxyphenyl)-6-(phenylsulfonyl)imidazo[4,5-d]pyrrolo[2,3-b]pyridin-1(6H)-yl)pyrrolidine-1-carboxylate (0.3 g, 0.5 mmol) in 10 mL of dichloromethane, slowly add trifluoroacetic acid (0.6 g, 5.0 mmol) and then stir at room temperature for 12 h. The product was concentrated under vacuum to yield 3-(6-(phenylsulfonyl)-1-(pyrrolidin-3-yl)-1,6-dihydroimidazo[4,5-d]pyrrolo[2,3-b]pyridin-2-yl) phenol as a light yellow oil (0.2 g, 81%). The product can be used directly in the next step without further purification. HRMS (ESI): m/z [M+H]⁺. C₂₄H₂₂N₅O₃S calculated value 460.1438, measured value 460.1441.

Step 3: Dissolve (R)-2-(1H-imidazol-2-yl)-6-(phenylsulfonyl)-1-(pyrrolidin-3-yl)-1,6-dihydroimidazo[4,5-d]pyrrolo[2,3-b]pyridine (0.2 g, 0.4 mmol) in 10 mL of tetrahydrofuran, add carbonyl diimidazole (0.2 g, 1.0 mmol) and then slowly add trifluoromethyl ethylamine (0.2 g, 1.0 mmol) dropwise. Then, stir the mixture at room temperature for 3 h. Monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield 3-(2-(3-hydroxyphenyl)-6-(phenylsulfonyl)imidazo[4,5-d]pyrrolo[2,3-b]pyridin-1(6H)-yl)-N-(2,2,2-trifluoroethyl)pyrrolidine-1-carboxamide as a pale yellow oil (0.2 g, 79%). The product can be used directly in the next step without further purification. HRMS (EST): m/z [M+H]⁺. C₂₇H₂₄F₃N₆O₄S calculated value 585.1526, measured value 585.1531.

Step 4: Dissolve 3-(2-(3-hydroxyphenyl)-6-(phenylsulfonyl)imidazo[4,5-d]pyrrolo[2,3-b]pyridin-1(6H)-yl)-N-(2,2,2-trifluoroethyl)pyrrolidine-1-carboxamide (0.2 g, 0.3 mmol) in a mixture of 5 mL of tetrahydrofuran and 5 mL of methanol, followed by adding 5 mL of 1M sodium hydroxide. Stir for 5 h at room temperature. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS34: 3-(2-(3-hydroxyphenyl)imidazo[4,5-d]pyrrolo[2,3-b]pyridin-1(6H)-yl)-N-(2,2,2-trifluoroethyl)pyrrolidine-1-carboxamide (0.1 g, 66%). ¹ HNMR (300 MHz, DMSO-d₆): δ=12.04 (s, 1H), 8.69 (s, 1H), 7.56 (t, J=3.0 Hz, 1H), 7.43 (t, J=7.5 Hz, 1H), 7.15 (t, J=4.5 Hz, 2H), 6.99-7.08 (m, 2H), 6.43 (d, J=3.0 Hz, 1H), 5.46 (t, J=9.0 Hz, 1H), 3.86-3.98 (m, 6H), 2.71-2.78 (m, 1H), 2.30-2.41 (m, 1H), 1.26 (s, 1H) ppm; ¹³ C NMR (75 MHz, DMSO-d₆) δ 158.03, 156.37, 152.81, 152.40, 144.80, 136.37, 135.20, 132.31, 131.66, 130.50, 125.03, 120.61, 117.57, 117.33, 116.78, 111.67, 105.18, 98.76, 91.18, 76.29, 73.64, 73.34, 67.45, 30.10 ppm; HRMS (ESI): m/z [M+H]⁺. C₂₁H₂₀F₃N₆O₂ calculated value 445.1594, measured value 445.1459.

### Example 35

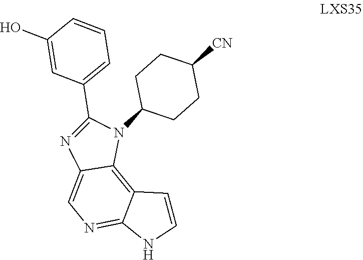 [SMILES: N#C[C@H]1CC[C@@H](n2c(-c3cccc(O)c3)nc3cnc4[nH]ccc4c32)CC1]

### trans-4-(2-(3-Hydroxyphenyl)imidazo[4,5-d]pyrrolo[2,3-b]pyridin-1(6H)-yl)cyclohexanecarbonitrile

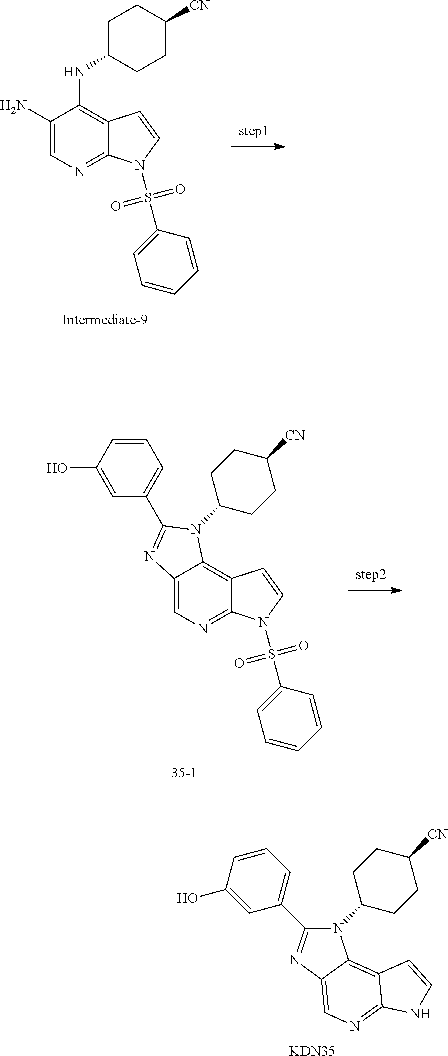

Step 1: Dissolve trans-4-((5-amino-1-(phenylsulfonyl)-1H-pyrrolo[2,3-b]pyridin-4-yl)amino)cyclohexanecarbonitrile (0.3 g, 0.8 mmol) in 10 mL of DMF, add Na₂S₂O₅ (0.8 g, 4 mmol) followed by adding 3-hydroxybenzaldehyde (0.2 g, 1.6 mmol) dropwise, then raise the temperature to 90° C. and stir for 12 hours. The reaction was monitored for completion by TLC. Saturated sodium bicarbonate was added until the reaction solution was weakly basic, and the organic phase was separated. The aqueous phase was extracted twice with dichloromethane. The organic phases were combined and washed with saturated saline, dried over anhydrous sodium sulfate and concentrated under vacuum to yield trans-4-(2-(3-hydroxyphenyl)-6-(phenylsulfonyl)imidazo[4,5-d]pyrrolo[2,3-b]pyridin-1(6H)-yl)cyclohexanecarbonitrile as a yellow oil (0.3 g, 79%). HRMS (ESI): m/z [M+H]⁺. C₂₇H₂₄N₅O₃S calculated value 498.1594, measured value 498.1588.

Step 2: Dissolve trans-4-(2-(3-hydroxyphenyl)-6-(phenylsulfonyl)imidazo[4,5-d]pyrrolo[2,3-b]pyridin-1(6H)-yl)cyclohexanecarbonitrile (0.3 g, 0.6 mmol) in a solvent mixture of 5 mL tetrahydrofuran and 5 mL methanol. Then add 5 mL of 1 M sodium hydroxide and stir at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS35: trans-4-(2-(3-Hydroxyphenyl)imidazo[4,5-d]pyrrolo[2,3-b]pyridin-1(6H)-yl)cyclohexanecarbonitrile (0.1 g, 46%). ¹HNMR (300 MHz, DMSO-d₆): δ=11.98 (s, 1H), 9.86 (s, 1H), 8.64 (s, 1H), 7.54 (t, J=3.0 Hz, 1H), 7.41 (t, J=7.5 Hz, 2H), 7.10 (t, J=4.5 Hz, 2H), 6.98-7.01 (m, 1H), 6.87 (s, 1H), 4.48-4.56 (m, 1H), 3.21 (s, 1H), 3.19 (s, 1H), 2.41 (t, J-=12.0 Hz, 2H), 2.26 (d, J=12.0 Hz, 2H), 2.02 (t, J=4.5 Hz, 2H), 1.67-1.79 (m, 2H) ppm; ¹³ C NMR (75 MHz, DMSO-d₆) δ 157.90, 152.05, 144.92, 136.19, 135.11, 132.69, 132.29, 130.35, 124.39, 123.15, 120.52, 117.15, 116.76, 104.60, 100.64, 55.37, 54.78, 49.05, 29.34, 28.75, 26.27 ppm; HRMS (ESI): m/z [M+H]⁺. C₂₁H₂₀N₅O calculated value 358.1662, measured value 358.1655.

### Example 36

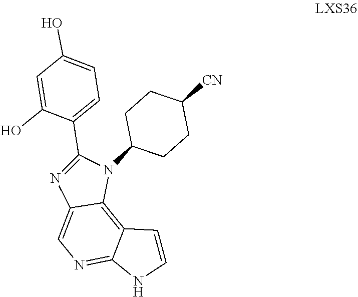 [SMILES: N#C[C@H]1CC[C@@H](n2c(-c3ccc(O)cc3O)nc3cnc4[nH]ccc4c32)CC1]

### trans-4-(2-(2,4-Dihydroxyphenyl)imidazo[4,5-d]pyrrolo[2,3-b]pyridin-1(6H)-yl)cyclohexanecarbonitrile

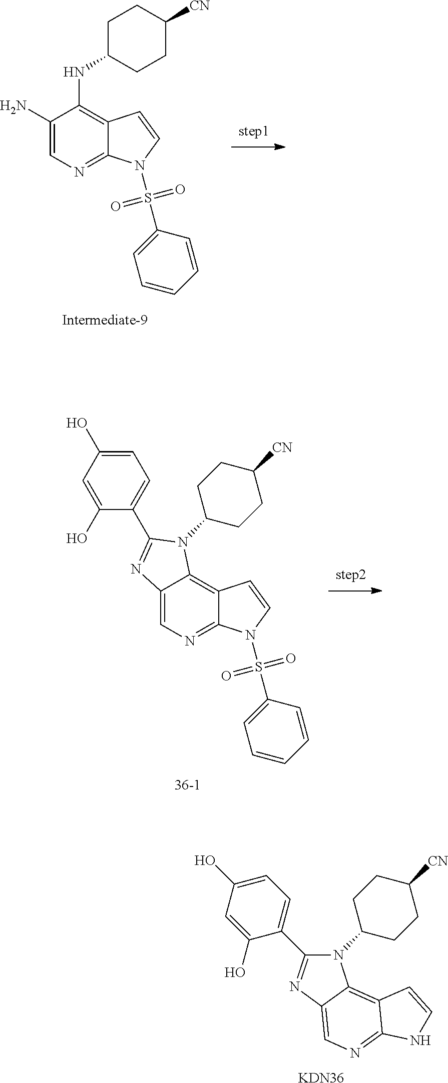

Step 1: Dissolve trans-4-((5-amino-1-(phenylsulfonyl)-1H-pyrrolo[2,3-b]pyridin-4-yl)amino)cyclohexanecarbonitrile (0.3 g, 0.8 mmol) in 10 mL of DMF, add Na₂S₂O₅ (0.8 g, 4 mmol) followed by adding 2,4-dihydroxybenzaldehyde (0.2 g, 1.6 mmol) dropwise. Stir for 12 h at 90° C. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield trans-4-(2-(2,4-dihydroxyphenyl)-6-(phenylsulfonyl)imidazo[4,5-d]pyrrolo[2,3-b]pyridin-1(6H)-yl)cyclohexanecarbonitrile as a yellow oil (0.3 g, 77%). HRMS (ESI): m/z [M+H]⁺. C₂₇H₂₄N₅O₄S calculated value 514.1544, measured value 514.1560.

Step 2: Trans-4-(2-(2,4-dihydroxyphenyl)-6-(phenylsulfonyl)imidazo[4,5-d]pyrrolo[2,3-b]pyridin-1(6H)-yl)cyclohexanecarbonitrile (0.3 g, 0.6 mmol) was dissolved in a mixture of 5 mL of tetrahydrofuran and 5 mL of methanol, and then stirred at room temperature for 5 h after the addition of 5 mL of 1M sodium hydroxide. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS36: trans-4-(2-(2,4-Dihydroxyphenyl)imidazo[4,5-d]pyrrolo[2,3-b]pyridin-1(6H)-yl)cyclohexanecarbonitrile (0.1 g, 46%). ¹ HNMR (300 MHz, DMSO-d₆): δ=11.92 (s, 1H), 8.58 (s, 1H), 7.49 (s, 1H), 7.19 (d, J=9.0 Hz, 1H), 6.84 (s, 1H), 6.58 (s, 1H), 6.41-6.44 (m, 1H), 4.23 (d, J=9.0 Hz, 1H), 3.14 (t, J=12.0 Hz, 1H), 2.79 (d, J=18.0 Hz, 1H), 2.36 (d, J=9.0 Hz, 2H), 2.26 (d, J=15.0 Hz, 2H), 2.02 (s, 1H), 1.62-1.74 (m, 2H), 1.18-1.26 (m, 2H) ppm; ¹³ C NMR (75 MHz, DMSO-d₆) δ 160.50, 157.73, 150.96, 144.77, 135.36, 133.04, 132.21, 124.09, 123.21, 109.32, 107.49, 104.42, 85.32, 73.67, 60.22, 54.89, 29.20, 28.95, 26.39, 14.55 ppm; HRMS (ESI): m/z [M+H]⁺. C₂₁H₂₀N₅O₂ calculated value 374.1612, measured value 374.1600.

### Example 37

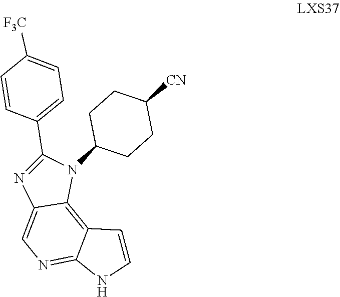 [SMILES: N#C[C@H]1CC[C@@H](n2c(-c3ccc(C(F)(F)F)cc3)nc3cnc4[nH]ccc4c32)CC1]

### trans-4-(2-(4-(Trifluoromethyl)phenyl)imidazo[4,5-d]pyrrolo[2,3-b]pyridin-1(6H)-yl)cyclohexanecarbonitrile

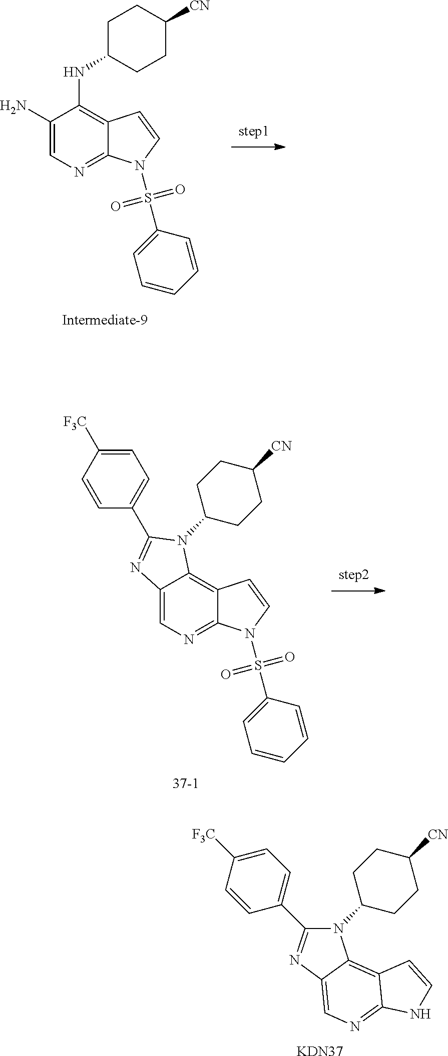

Step 1. Dissolve trans-4-((5-amino-1-(phenylsulfonyl)-1H-pyrrolo[2,3-b]pyridin-4-yl)amino)cyclohexanecarbonitrile (0.3 g, 0.8 mmol) in 10 mL of DMF, add Na₂S₂O₅ (0.8 g, 4 mmol) followed by adding 4-(trifluoromethyl)benzaldehyde (0.3 g, 1.6 mmol) dropwise. Stir for 12 h at 90° C. The reaction was monitored for completion by TLC. Saturated sodium bicarbonate was added until the reaction solution was weakly basic, and the organic phase was separated. The aqueous phase was extracted twice with dichloromethane. The organic phases were combined and washed with saturated saline, dried over anhydrous sodium sulfate and concentrated under vacuum to yield trans-4-(6-(phenylsulfonyl)-2-(4-(trifluoromethyl)phenyl)imidazo[4,5-d]pyrrolo[2,3-b]pyridin-1(6H)-yl) cyclohexanecarbonitrile as a yellow oil (0.3 g, 72%). HRMS (ESI): m/z [M+H]⁺. C₂₈H₂₃F₃N₅O₂S calculated value 550.1519, measured value 550.1521.

Step 2: Trans-4-(6-(phenylsulfonyl)-2-(4-(trifluoromethyl)phenyl)imidazo[4,5-d]pyrrolo[2,3-b]pyridin-1(6H)-yl) cyclohexanecarbonitrile (0.3 g, 0.5 mmol) was dissolved in a mixture of 5 mL of tetrahydrofuran and 5 mL of methanol. 5 mL of 1 M sodium hydroxide was added and then stirred at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS37: trans-4-(2-(4-(trifluoromethyl)phenyl)imidazo[4,5-d]pyrrolo[2,3-b]pyridin-1(6H)-yl)cyclohexanecarbonitrile (0.1 g, 45%).). ¹ HNMR (300 MHz, DMSO-d₆): δ=12.04 (s, 1H), 8.68 (s, 1H), 7.97 (d, J=3.0 Hz, 4H), 7.56 (t, J=3.0 Hz, 1H), 6.89 (s, 1H), 4.45-4.53 (m, 1H), 3.17 (t, J=13.5 Hz, 1H), 2.40 (t, J=10.5 Hz, 2H), 2.23 (d, J=12.0 Hz, 2H), 2.03 (t, J=10.5 Hz, 2H), 1.74-1.86 (m, 2H), 1.25 (s, 1H) ppm; ¹³ C NMR (75 MHz, DMSO-d₆) δ 150.58, 145.02, 136.44, 135.27, 132.89, 131.00, 130.63, 129.90, 126.38, 126.10, 124.55, 123.21, 104.58, 100.85, 72.19, 54.92, 29.32, 28.50, 26.22 ppm; HRMS (ESI): m/z [M+H]⁺. C₂₂H₁₉F₃N₅ calculated value 410.1587, measured value 410.1590.

### Example 38

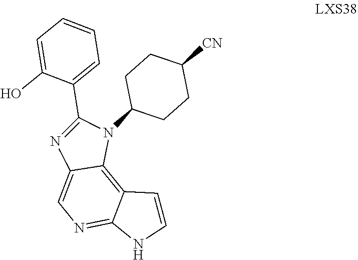 [SMILES: N#C[C@H]1CC[C@@H](n2c(-c3ccccc3O)nc3cnc4[nH]ccc4c32)CC1]

### trans-4-(2-(2-Hydroxyphenyl)imidazo[4,5-d]pyrrolo[2,3-b]pyridin-1(6H)-yl)cyclohexanecarbonitrile

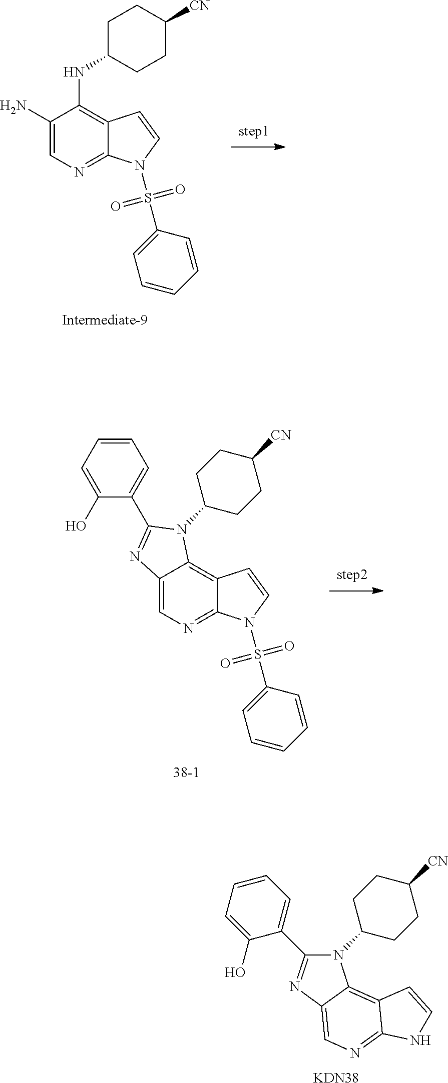

Step 1. Dissolve trans-4-((5-amino-1-(phenylsulfonyl)-1H-pyrrolo[2,3-b]pyridin-4-yl)amino)cyclohexanecarbonitrile (0.3 g, 0.8 mmol) in 10 mL of DMF, add Na₂S₂O₅ (0.8 g, 4 mmol) followed by adding 2-hydroxybenzaldehyde (0.2 g, 1.6 mmol) dropwise, raise the temperature to 90° C. and stir for 12 hours. The reaction was monitored for completion by TLC. Saturated sodium bicarbonate was added until the reaction solution was weakly basic, and the organic phase was separated. The aqueous phase was extracted twice with dichloromethane. The organic phases were combined and washed with saturated saline, dried over anhydrous sodium sulfate and concentrated under vacuum to yield trans-4-(2-(2-hydroxyphenyl)-6-(phenylsulfonyl)imidazo[4,5-d]pyrrolo[2,3-b]pyridin-1(6H)-yl)cyclohexanecarbonitrile as a yellow oil (0.3 g, 79%). HRMS (ESI): m/z [M+H]⁺. C₂₇H₂₄N₅O₃S calculated value 498.1594, measured value 498.1600.

Step 2: Dissolve trans-4-(2-(2-hydroxyphenyl)-6-(phenylsulfonyl)imidazo[4,5-d]pyrrolo[2,3-b]pyridin-1(6H)-yl)cyclohexanecarbonitrile (0.3 g, 0.6 mmol) in a solvent mixture of 5 mL tetrahydrofuran and 5 mL methanol, add 5 mL of 1 M sodium hydroxide and stir at room temperature for 5 h. Monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS38: trans-4-(2-(2-Hydroxyphenyl)imidazo[4,5-d]pyrrolo[2,3-b]pyridin-1(6H)-yl)cyclohexanecarbonitrile (0.1 g, 46%). ¹HNMR (300 MHz, DMSO-d₆): δ=11.94 (s, 1H), 10.17 (s, 1H), 8.62 (s, 1H), 7.52 (t, J=3.0 Hz, 1H), 7.40-7.46 (m, 2H), 6.98-7.08 (m, 2H), 6.87 (d, J=3.0 Hz, 1H), 4.18-4.26 (m, 1H), 3.14 (t, J=12.0 Hz, 1H), 2.38 (t, J=12.0 Hz, 2H), 2.26 (d, J=10.5 Hz, 2H), 1.60-1.72 (m, 2H), 1.18-1.26 (m, 2H) ppm; ¹³ C NMR (75 MHz, DMSO-d₆) δ 156.49, 150.28, 144.85, 135.95, 135.45, 132.47, 132.24, 131.75, 124.19, 123.16, 119.68, 118.54, 116.23, 104.41, 100.53, 72.30, 55.03, 29.22, 28.89, 26.37 ppm: HRMS (ESI): m/z [M+H]⁺. C₂₁H₂₀N₅O calculated value 358.1662, measured value 358.1658.

### Example 39

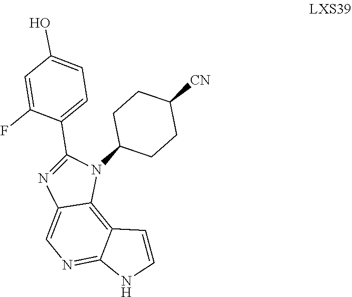 [SMILES: N#C[C@H]1CC[C@@H](n2c(-c3ccc(O)cc3F)nc3cnc4[nH]ccc4c32)CC1]

### trans-4-(2-(2-Fluoro-4-hydroxyphenyl)imidazo[4,5-d]pyrrolo[2,3-b]pyridin-1(6H)-yl)cyclohexanecarbonitrile

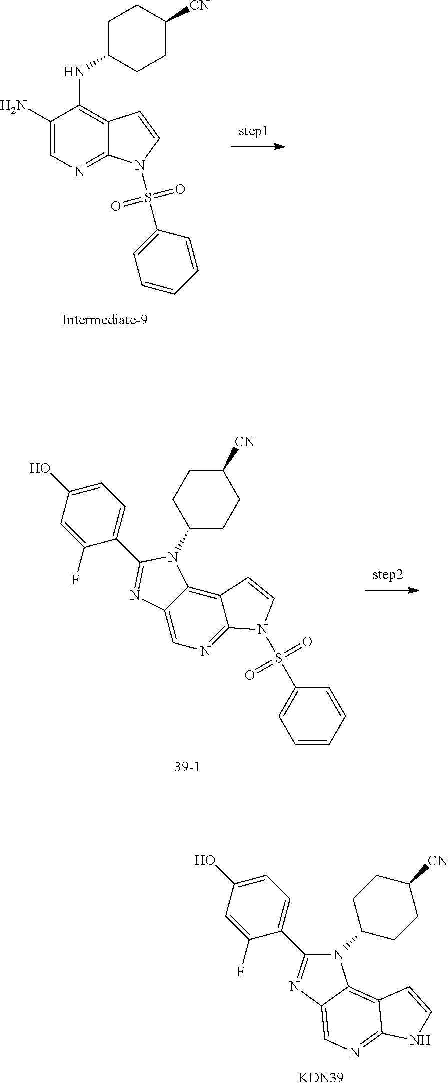

Step 1: Dissolve trans-4-((5-amino-1-(phenylsulfonyl)-1H-pyrrolo[2,3-b]pyridin-4-yl)amino)cyclohexanecarbonitrile (0.3 g, 0.8 mmol) in 10 mL of DMF, add Na₂S₂O₅ (0.8 g, 4 mmol) followed by the addition of 2-fluoro-4-hydroxybenzaldehyde (0.2 g, 1.6 mmol) dropwise and then stir for 12 h at 90° C. The reaction was monitored for completion by TLC. Saturated sodium bicarbonate was added until the reaction solution was weakly basic, and the organic phase was separated. The aqueous phase was extracted twice with dichloromethane. The organic phases were combined and washed with saturated saline, dried over anhydrous sodium sulfate and concentrated under vacuum to yield trans-4-(2-(2-fluoro-4-hydroxyphenyl)-6-(phenylsulfonyl)imidazo[4,5-d]pyrrolo[2,3-b]pyridin-1(6H)-yl) cyclohexanecarbonitrile as a pale yellow oil (0.3 g, 77%). HRMS (ESI): m/z [M+H]⁺. C₂₇H₂₃FN₅O₃S calculated value 516.1500, measured value 516.1511.

Step 2: Dissolve trans-4-(2-(2-fluoro-4-hydroxyphenyl)-6-(phenylsulfonyl)imidazo[4,5-d]pyrrolo[2,3-b]pyridin-1(6H)-yl) cyclohexanecarbonitrile (0.3 g, 0.6 mmol) in a mixture of 5 mL of tetrahydrofuran and 5 mL of methanol, add 5 mL of 1 M sodium hydroxide and then stir for 5 h at room temperature. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS39: trans-4-(2-(2-Fluoro-4-hydroxyphenyl)imidazo[4,5-d]pyrrolo[2,3-b]pyridin-1(6H)-yl)cyclohexanecarbonitrile (0.1 g, 46%).). ¹ HNMR (300 MHz, DMSO-d₆): δ=11.99 (s, 1H), 8.63 (s, 1H), 7.54 (t, J=3.0 Hz, 1H), 7.43-7.49 (m, 1H), 6.88 (d, J=3.0 Hz, 1H), 6.82-6.85 (m, 1H), 6.80 (d, J=3.0 Hz, 1H), 4.24 (d, J=12.0 Hz, 1H), 3.13 (t, J=13.5 Hz, 1H), 2.34 (d, J 12.0 Hz, 2H), 2.23 (d, J=12.0 Hz, 2H), 2.02 (s, 1H), 1.94 (d, J=9.0 Hz, 2H), 1.70-1.82 (m, 2H), 1.18-1.26 (m, 1H) ppm; ¹³ C NMR (75 MHz, DMSO-d₆) δ 160.11, 159.75, 153.77, 148.62, 142.15, 130.54, 129.04, 127.16, 122.75, 120.77, 116.16, 115.64, 112.04, 104.54, 99.38, 65.93, 30.88, 26.91, 23.69 ppm: HRMS (ESI): m/z [M+H]⁺. C₂₁H₁₉FN₅O calculated value 376.1568, measured value 376.1562.

### Example 40

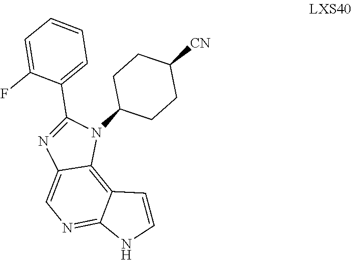 [SMILES: N#C[C@H]1CC[C@@H](n2c(-c3ccccc3F)nc3cnc4[nH]ccc4c32)CC1]

### trans-4-(2-(2-Fluorophenyl)imidazo[4,5-d]pyrrolo[2,3-b]pyridin-1(6H)-yl)cyclohexanecarbonitrile

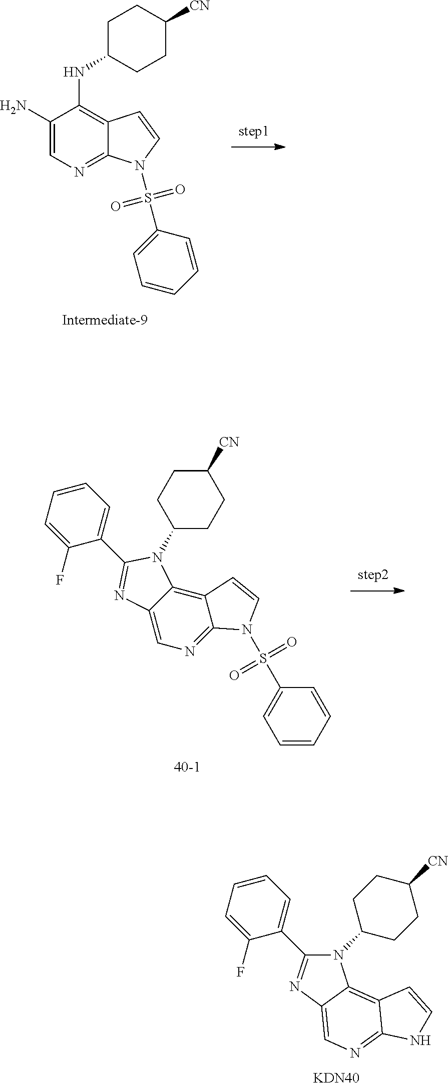

Step 1: Dissolve trans-4-((5-amino-1-(phenylsulfonyl)-1H-pyrrolo[2,3-b]pyridin-4-yl)amino)cyclohexanecarbonitrile (0.3 g, 0.8 mmol) in 10 mL of DMF, add Na₂S₂O₅ (0.8 g, 4 mmol) followed by the addition of 2-fluorobenzaWdehyde (0.2 g, 1.6 mmol) dropwise, raise the temperature to 90° C. and stir for 12 hours. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield trans-4-(2-(2-fluorophenyl)-6-(phenylsulfonyl)imidazo[4,5-d]pyrrolo[2,3-b]pyridin-1(6H)-yl)cyclohexanecarbonitrile as a pale yellow oil (0.3 g, 79%). HRMS (EST): m/z [M+H]⁺. C₂₇H₂₁FN₅O₂S calculated value 500.1551, measured value 500.1563.

Step 2: Dissolve trans-4-(2-(2-fluorophenyl)-6-(phenylsulfonyl)imidazo[4,5-d]pyrrolo[2,3-b]pyridin-1(6H)-yl) cyclohexanecarbonitrile (0.3 g, 0.6 mmol) in a solvent mixture of 5 mL tetrahydrofuran and 5 mL methanol, add 5 mL of 1 M sodium hydroxide and then stir at room temperature for 5 h. Monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum. The residue was purified using a silica gel column (petroleum ether:ethyl acetate=1:1) to yield LXS40: trans-4-(2-(2-Fluorophenyl)imidazo[4,5-d]pyrrolo[2,3-b]pyridin-1(6H)-yl)cyclohexanecarbonitrile (0.1 g, 46%). ¹HNMR (300 MHz, DMSO-d₆): δ=12.06 (s, 1H), 8.70 (s, 1H), 7.70 (t, J=6.0 Hz, 2H), 7.57 (s, 1H), 7.49 (t, J=9.0 Hz, 2H), 6.91 (s, 1H), 4.27 (s, 1H), 3.13 (s, 1H), 2.35 (d, J=12.0 Hz, 2H), 2.24 (d, J=9.0 Hz, 2H), 2.01 (d, J=6.0 Hz, 2H), 1.75 (d, J=12.0 Hz, 2H) ppm; ¹³ C NMR (75 MHz, DMSO-d₆) δ 162.07, 158.80, 146.49, 145.00, 136.27, 135.41, 133.08, 132.92, 132.42, 125.44, 124.54, 123.11, 119.40, 119.19, 116.62, 116.34, 104.40, 100.51, 60.22, 55.18, 29.42, 28.59, 26.29, 14.51 ppm; HRMS (EST): m/z [M+H]⁺. C₂₁H¹⁹FN₅ calculated value 360.1619, measured value 360.1615.

### Example 41

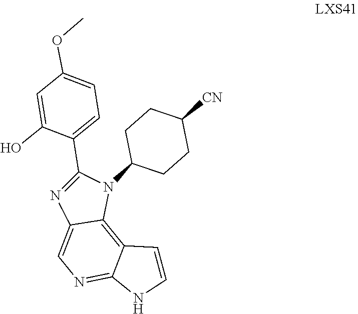 [SMILES: COc1ccc(-c2nc3cnc4[nH]ccc4c3n2[C@H]2CC[C@@H](C#N)CC2)c(O)c1]

### trans-4-(2-(2-Hydroxy-4-methoxyphenyl)imidazo[4,5-d]pyrrolo[2,3-b]pyridin-1(6H)-yl)cyclohexanecarbonitrile

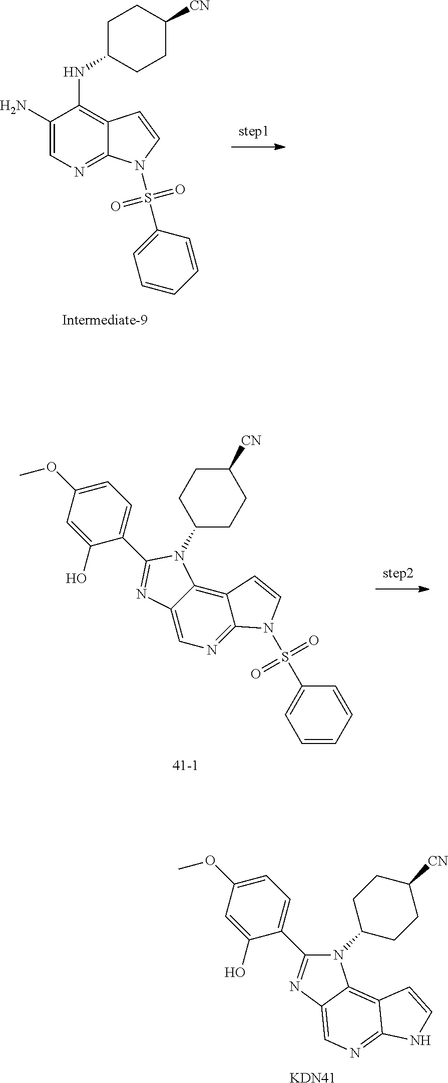

Step 1: Dissolve trans-4-((5-amino-1-(phenylsulfonyl)-1H-pyrrolo[2,3-b]pyridin-4-yl)amino)cyclohexanecarbonitrile (0.3 g, 0.8 mmol) in 10 mL of DMF, add Na₂S₂O₅ (0.8 g, 4 mmol) followed by adding 2-fluoro 4-methoxybenzaldehyde (0.2 g, 1.6 mmol) dropwise, and then stir for 12 h at 90° C. The reaction was monitored for completion by TLC. Saturated sodium bicarbonate was added until the reaction solution was weakly basic, and the organic phase was separated. The aqueous phase was extracted twice with dichloromethane. The organic phases were combined and washed with saturated saline, dried over anhydrous sodium sulfate and concentrated under vacuum to yield trans-4-(2-(2-hydroxy-4-methoxyphenyl)-6-(phenylsulfonyl)imidazo[4,5-d]pyrrolo[2,3-b]pyridin-1(6H)-yl) cyclohexanecarbonitrile as a yellow oil (0.3 g, 75%). HRMS (ESI): m/z [M+H]⁺. C₂₈H₂₆N₅O₄S calculated value 528.1700, measured value 528.1711.

Step 2: Trans-4-(2-(2-hydroxy-4-methoxyphenyl)-6-(phenylsulfonyl)imidazo[4,5-d]pyrrolo[2,3-b]pyridin-1(6H)-yl) cyclohexanecarbonitrile (0.3 g, 0.6 mmol) was dissolved in a mixture of 5 mL of tetrahydrofuran and 5 mL of methanol, and then stirred at room temperature for 5 h after the addition of 5 mL of 1 M sodium hydroxide. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS41: trans-4-(2-(2-hydroxy-4-methoxyphenyl)imidazo[4,5-d]pyrrolo[2,3-b]pyridin-1(6H)-yl)cyclohexanecarbonitrile (0.1 g, 46%), 46%). ¹ HNMR (300 MHz, DMSO-d₆): δ=11.91 (s, 1H), 10.26 (s, 1H), 8.60 (s, 1H), 7.51 (t, J=3.0 Hz, 1H), 7.34 (d, J=9.0 Hz, 1H), 6.86 (d, J=3.0 Hz, 1H), 6.58-6.61 (m, 2H), 4.24 (t, J=13.5 Hz, 1H), 3.83 (s, 3H), 3.15 (t, J=12.0 Hz, 1H), 2.39 (t, J=12.0 Hz, 2H), 2.25 (d, J=12.0 Hz, 2H), 2.01 (d, J=12.0 Hz, 2H), 1.62-1.74 (m, 2H) ppm; ¹³ C NMR (75 MHz, DMSO-d₆) δ 162.03, 157.72, 150.38, 144.83, 135.83, 135.37, 133.21, 132.24, 124.14, 123.19, 111.08, 105.80, 104.42, 101.63, 55.60, 54.94, 29.20, 28.92, 26.38 ppm; HRMS (EST): m/z [M+H]⁺. C₂₂H₂₂N₅O₂ calculated value 388.1768, measured value 366.1782.

### Example 42

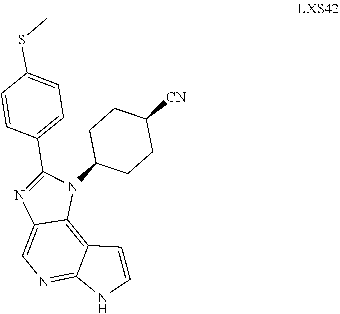 [SMILES: CSc1ccc(-c2nc3cnc4[nH]ccc4c3n2[C@H]2CC[C@@H](C#N)CC2)cc1]

### trans-4-(2-(4-(Methylthio)phenyl)imidazo[4,5-d]pyrrolo[2,3-b]pyridin-1(6H)-yl)cyclohexanecarbonitrile

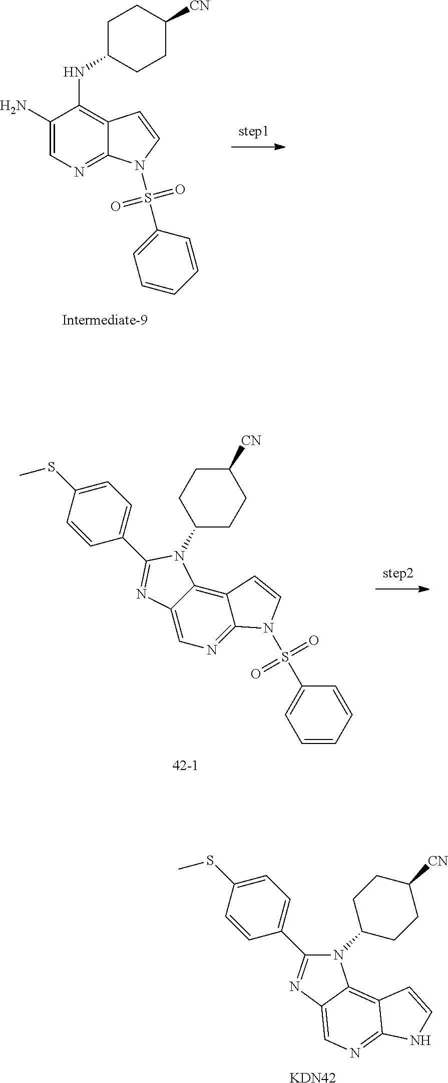

Step 1: Dissolve trans-4-((5-amino-1-(phenylsulfonyl)-1H-pyrrolo[2,3-b]pyridin-4-yl)amino)cyclohexanecarbonitrile (0.3 g, 0.8 mmol) in 10 mL of DMF, add Na₂S₂O₅ (0.8 g, 4 mmol) followed by adding 4-(methylthio)benzaldehyde (0.2 g, 1.6 mmol) dropwise. Stir for 12 h at 90° C. The reaction was monitored for completion by TLC. Saturated sodium bicarbonate was added until the reaction solution was weakly basic, and the organic phase was separated. The aqueous phase was extracted twice with dichloromethane. The organic phases were combined and washed with saturated saline, dried over anhydrous sodium sulfate and concentrated under vacuum to yield trans-4-(2-(4-(methylthio)phenyl)-6-(phenylsulfonyl)imidazo[4,5-d]pyrrolo[2,3-b]pyridin-1(6H)-yl) cyclohexanecarbonitrile as a yellow oil (0.3 g, 75%). HRMS (ESI): m/z [M+H]⁺. C₂₈H₂₆N₅O₂S₂ calculated value 528.1522, measured value 528.1533.

Step 2: Trans-4-(2-(4-(methylthio)phenyl)-6-(phenylsulfonyl)imidazo[4,5-d]pyrrolo[2,3-b]pyridin-1(6H)-yl) cyclohexanecarbonitrile (0.3 g, 0.6 mmol) was dissolved in a mixture of 5 mL of tetrahydrofuran and 5 mL of methanol, 5 mL of 1 M sodium hydroxidewas added and then stirred at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate=1:1) to yield LXS42: trans-4-(2-(4-(methylthio)phenyl)imidazo[4,5-d]pyrrolo[2,3-b]pyridin-1(6H)-yl)cyclohexanecarbonitrile (0.1 g, 46%). ¹ HNMR (300 MHz, DMSO-d₆): δ=11.98 (s, 1H), 8.65 (s, 1H), 7.66 (d, J 9.0 Hz, 2H), 7.54 (t, J=3.0 Hz, 1H), 7.48 (d, J=9.0 Hz, 2H), 6.88 (d, J=3.0 Hz, 1H), 4.49 (t, J=13.5 Hz, 1H), 3.17 (t, J=12.0 Hz, 1H), 2.61 (s, 3H), 2.45 (t, J=12.0 Hz, 2H), 2.25 (d, J=12.0 Hz, 2H), 2.03 (d, J=6.0 Hz, 2H), 1.71-1.83 (m, 2H) ppm; ¹³ C NMR (75 MHz, DMSO-d₆) δ 153.77, 148.43, 142.89, 139.43, 129.04, 127.77, 127.32, 127.17, 127.01, 122.74, 120.73, 115.64, 99.49, 65.93, 31.96, 26.94, 24.68, 14.88 ppm; HRMS (ESI): m/z [M+H]⁺. C₂₂H₂₂N₅O₂ calculated value 388.1590, measured value 388.1599.

### Example 43

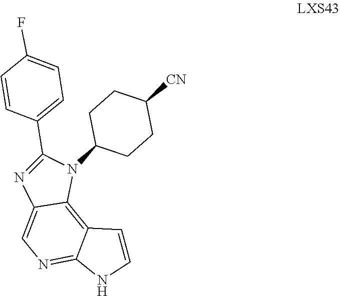 [SMILES: N#C[C@H]1CC[C@@H](n2c(-c3ccc(F)cc3)nc3cnc4[nH]ccc4c32)CC1]

### trans-4-(2-(4-Fluorophenyl)imidazo[4,5-d]pyrrolo[2,3-b]pyridin-1(6H)-yl)cyclohexanecarbonitrile

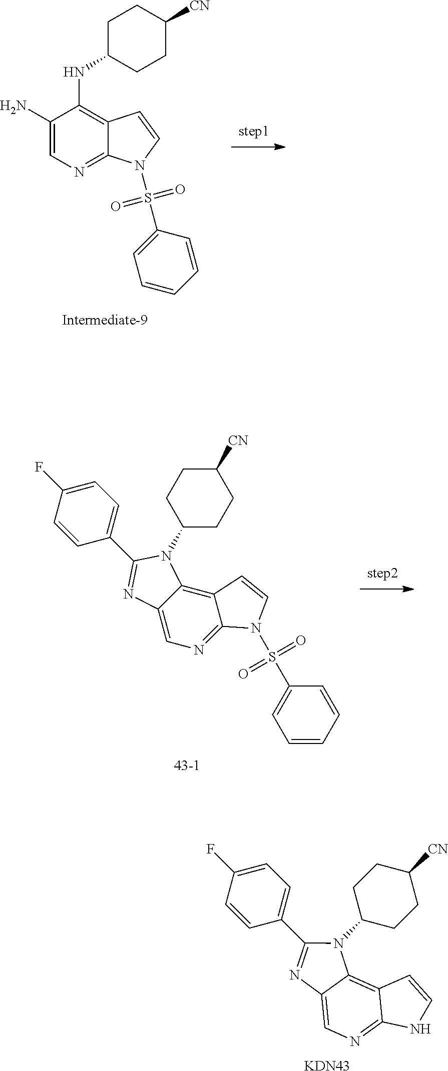

Step 1. Dissolve trans-4-((5-amino-1-(phenylsulfonyl)-1H-pyrrolo[2,3-b]pyridin-4-yl)amino)cyclohexanecarbonitrile (0.3 g, 0.8 mmol) in 10 mL of DMF, add Na₂S₂O₅ (0.8 g, 4 mmol) followed by the addition of 4-fluorobenzaldehyde (0.2 g, 1.6 mmol) dropwise, raise the temperature to 90° C. and stir for 12 hours. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield trans-4-(2-(4-fluorophenyl)-6-(phenylsulfonyl)imidazo[4,5-d]pyrrolo[2,3-b]pyridin-1(6H)-yl) cyclohexanecarbonitrile as a yellow oil (0.3 g, 79%). HRMS (ESI): m/z [M+H]⁺. C₂₇H₂₃FN₅O₂S calculated value 500.1551, measured value 500.1559.

Step 2: Dissolve trans-4-(2-(4-fluorophenyl)-6-(phenylsulfonyl)imidazo[4,5-d]pyrrolo[2,3-b]pyridin-1(6H)-yl) cyclohexanecarbonitrile (0.3 g, 0.6 mmol) in a mixture of 5 mL of tetrahydrofuran and 5 mL of methanol, followed by the addition of 5 mL of 1M sodium hydroxide. Then, stir the mixture at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS43: trans-4-(2-(4-Fluorophenyl)imidazo[4,5-d]pyrrolo[2,3-b]pyridin-1(6H)-yl)cyclohexanecarbonitrile (0.1 g, 46%). ¹HNMR (300 MHz, DMSO-d₆): δ=11.99 (s, 1H), 8.65 (s, 1H), 7.74-7.79 (m, 2H), 7.54 (t, J=3.0 Hz, 1H), 7.46 (t, J=9.0 Hz, 2H), 6.88 (d, J=3.0 Hz, 1H), 4.44 (t, J=12.0 Hz, 1H), 3.15 (t, J=12.0 Hz, 1H), 2.34-2.47 (m, 2H), 2.24 (d, J=12.0 Hz, 2H), 2.04 (d, J=9.0 Hz, 2H), 1.70-1.81 (m, 2H) ppm; ¹³ C NMR (75 MHz, DMSO-d₆) δ 162.99, 153.74, 148.65, 142.11, 129.19, 129.03, 127.43, 126.22, 122.74, 120.75, 116.43, 115.64, 99.40, 65.93, 30.82, 26.94, 21.54 ppm, HRMS (ESI): m/z [M+H]⁺. C₂₁H₁₉FN₅ calculated value 360.1619, measured value 360.1683.

### Example 44

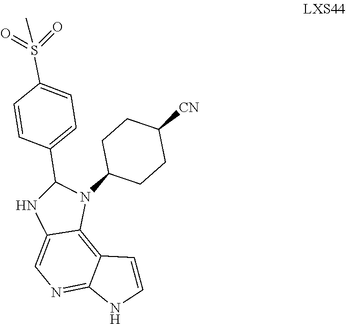 [SMILES: CS(=O)(=O)c1ccc(C2Nc3cnc4[nH]ccc4c3N2[C@H]2CC[C@@H](C#N)CC2)cc1]

### trans-4-(2-(4-(Methylsulfonyl)phenyl)-2,3-dihydroimidazo[4,5-d]pyrrolo[2,3-b]pyridin-1(6H)-yl)cyclohexanecarbonitrile

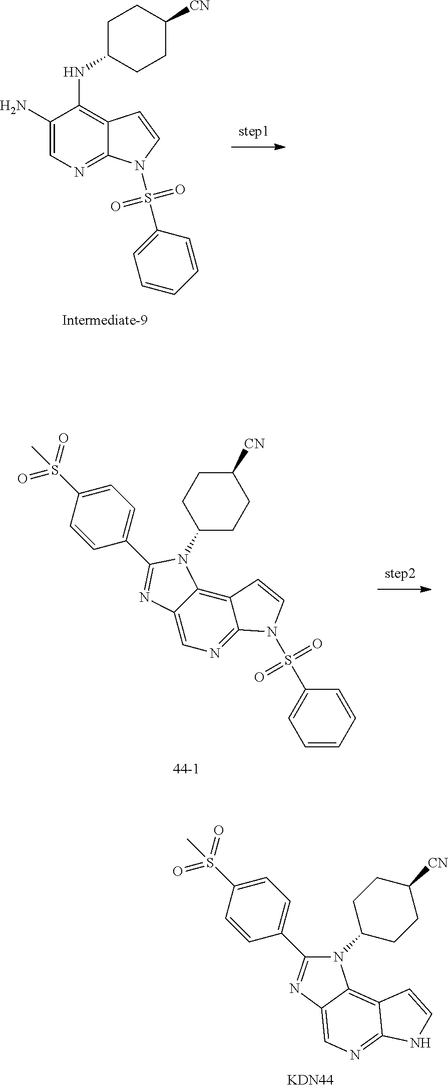

Step 1: Dissolve trans-4-((5-amino-1-(phenylsulfonyl)-1H-pyrrolo[2,3-b]pyridin-4-yl)amino)cyclohexanecarbonitrile (0.3 g, 0.8 mmol) in 10 mL of DMF, add Na₂S₂O₅ (0.8 g, 4 mmol) followed by the addition of 4-methylsulfonylbenzaldehyde (0.3 g, 1.6 mmol) dropwise. The reaction was monitored for completion by TLC. Saturated sodium bicarbonate was added until the reaction solution was weakly basic, and the organic phase was separated, the aqueous phase was extracted twice with dichloromethane. The organic phases were combined and washed with saturated saline, dried over anhydrous sodium sulfate and concentrated under vacuum to yield trans-4-(2-(4-(methylsulfonyl)phenyl)-6-(phenylsulfonyl)-2,3-dihydroimidazo[4,5-d]pyrrolo[2,3-b]pyridin-1(6H)-yl)cyclohexanecarbonitrile as a yellow oil (0.3 g, 70%). HRMS (ESI): m/z [M+H]⁺. C₂₈H₂₈N₅O₄S₂ calculated value 562.1577, measured value 562.1588.

Step 2: Trans-4-(2-(4-(methylsulfonyl)phenyl)-6-(phenylsulfonyl)-2,3-dihydroimidazo[4,5-d]pyrrolo[2,3-b]pyridin-1(6H)-yl) cyclohexanecarbonitrile (0.3 g, 0.5 mmol) was dissolved in a solvent mixture of 5 mL of tetrahydrofuran and 5 mL of methanol, followed by the addition of 5 mL of 1 M sodium hydroxide. The mixture is then stirred at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhvdrous sodium sulfate and concentrated under vacuum, and the residue was purified using a silica gel column (petroleum ether:ethyl acetate=1:1) to yield LXS44: trans-4-(2-(4-(Methylsulfonyl)phenyl)-2,3-dihydroimidazo[4,5-d]pyrrolo[2,3-b]pyridin-1(6H)-yl)cyclohexanecarbonitrile (0.2 g, 89%), 1 HNMR (300 MHz, DMSO-d₆): δ=11.40 (s, 1H), 8.93 (s, 1H), 8.22 (d, J=9.0 Hz, 2H), 8.14 (s, 1H), 8.06 (d, J=6.0 Hz, 2H), 7.22 (t, J=3.0 Hz, 1H), 6.63 (d, J=3.0 Hz, 1H), 6.07 (d, J=9.0 Hz, 1H), 4.02-4.10 (m, 1H), 3.30 (s, 3H), 2.73-2.81 (m, 1H), 2.12 (d, J=9.0 Hz, 2H), 2.03 (s, 2H), 1.76-1.87 (m, 2H), 1.44-1.55 (m, 2H) ppm; ¹³ C NMR (75 MHz, DMSO-d₆) δ 144.28, 139.38, 138.74, 134.46, 131.18, 128.94, 128.11, 127.19, 123.38, 122.74, 108.17, 99.36, 83.56, 61.28, 47.74, 28.48, 26.94, 24.93 ppm; HRMS (ESI): m/z [M+H]⁺. C₂₂H₂₄N₅O₂S calculated value 422.1645, measured value 422.1679.

### Example 45

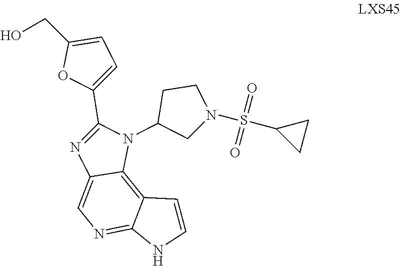 [SMILES: O=S(=O)(C1CC1)N1CCC(n2c(-c3ccc(CO)o3)nc3cnc4[nH]ccc4c32)C1]

### (5-(1-(1-(cyclopropylsulfonyl)pyrrolidin-3-yl)-1,6-dihydroimidazo[4,5-d]pyrrolo[2,3-b]pyridin-2-yl)furan-2-yl)methanol

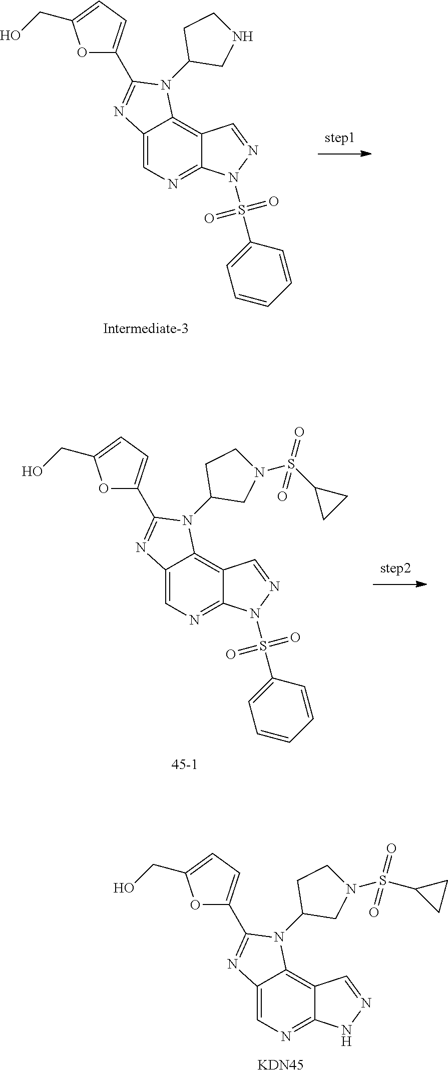

Step 1: Dissolve 5-(6-(phenylsulfonyl)-1-(pyrrolidin-3-yl)-1,6-dihydroimidazo[4,5-d]pyrrolo[2,3-b]pyridin-2-yl)furan-2-yl) methanol (0.3 g, 0.6 mmol) in 10 mL of tetrahydrofuran, add DIPEA (0.2 g, 1.2 mmol) and then slowly add cyclopropylsulfonyl chloride (0.1 g, 0.9 mmol) dropwise. Then raise the temperature to maintain reflux and stir for 3 h. Monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield (5-(6-(phenylsulfonyl)-1-(1-(cyclopropylsulfonyl)pyrrolidin-3-yl)-1,6-dihydroimidazo[4,5-d]pyrrolo[2,3-b]pyridin-2-yl) furan-2-yl) methanol as a yellow oil (0.3 g, 82%). The product can be used directly in the next step without further purification. HRMS (EST): m/z [M+H]⁺. C₂₆H₂₆N₅O₆S₂ calculated value 568.1319, measured value 568.1321.

Step 2: Dissolve (5-(6-(phenylsulfonyl)-1-(1-(cyclopropylsulfonyl)pyrrolidin-3-yl)-1,6-dihydroimidazo[4,5-d]pyrrolo[2,3-b]pyridin-2-yl) furan-2-yl) methanol (0.3 g, 0.5 mmol) in a solvent mixture of 5 mL oftetrahydrofuran and 5 mL of methanol, followed by the addition of 5 mL 1 M sodium hydroxide. Stir the mixture for 5 h at room temperature and the reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS45: (5-(1-(1-(cyclopropylsulfonyl)pyrrolidin-3-yl)-1,6-dihydroimidazo[4,5-d]pyrrolo[2,3-b]pyridin-2-yl)furan-2-yl)methanol (0.1 g, 44%). ¹ HNMR (300 MHz, DMSO-d₆): δ=12.09 (s, 1H), 8.67 (s, 1H), 7.61 (t, J=3.0 Hz, 1H), 7.19 (d, J=3.0 Hz, 1H), 6.85 (s, 1H), 6.63 (d, J=3.0 Hz, 1H), 5.80-5.92 (m, 1H), 5.51 (t, J=6.0 Hz, 1H), 4.58 (d, J=6.0 Hz, 2H), 4.04-4.10 (m, 1H), 3.81-3.90 (m, 2H), 3.58-3.67 (m, 1H), 2.89-2.97 (m, 1H), 2.71-2.80 (m, 2H), 1.26 (s, 1H), 1.06 (t, J=6.0 Hz, 4H) ppm: ^{—}C NMR (75 MHz, DMSO-d₆) δ 153.88, 151.18, 148.60, 144.53, 142.17, 129.04, 127.16, 120.88, 115.63, 107.95, 104.05, 99.38, 57.39, 56.43, 54.43, 49.75, 37.55, 24.66, 4.07 ppm; HRMS (ESI): m/z [M+H]⁺. C₂₀H₂₂N O₄S calculated value 428.1387, measured value 428.1384.

### Example 46

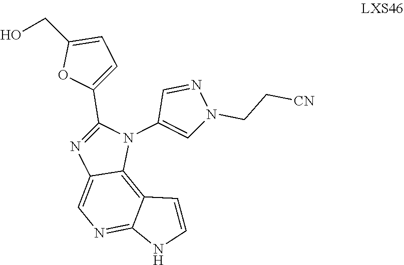 [SMILES: N#CCCn1cc(-n2c(-c3ccc(CO)o3)nc3cnc4[nH]ccc4c32)cn1]

### 3-(4-(2-(5-(Hydroxymethyl)furan-2-yl)imidazo[4,5-d]pyrrolo[2,3-b]pyridin-1(6H)-yl)-1H-pyrazol-1-yl)propanenitrile

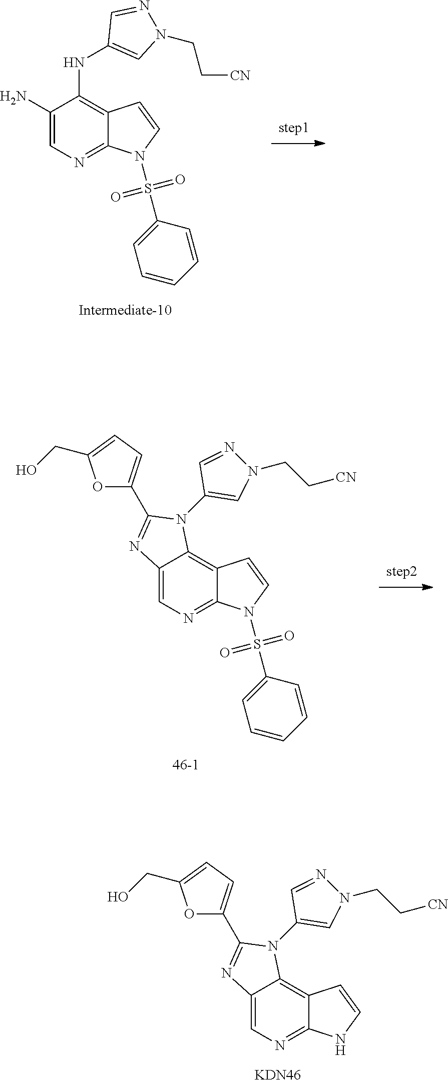

Step 1: Dissolve 3-(4-((5-amino-1-(phenylsulfonyl)-1H-pyrrolo[2,3-b]pyridin-4-yl)amino)-1H-pyrazo-1-yl)propanenitrile (0.3 g, 0.7 mmol) in 10 mL of DMF, add Na₂S₂O₅ (0.8 g, 4 mmol) followed by adding 5-hydroxymethylfurfural (0.2 g, 1.4 mmol) and then stir for 12 h at 90° C. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield 3-(4-(2-(5-(hydroxymethyl)furan-2-yl)-6-(phenylsulfonyl)imidazo[4,5-d]pyrrolo[2,3-b]pyridin-1(6H)-yl)-1H-pyrazol-1-yl)propanenitrile as a yellow oil (0.3 g, 79%). HRMS (ESI): m/z [M+H]⁺. C₂₅H₂₀N₇O₄S calculated value 514.1292, measured value 514.1299.

Step 2: Dissolve 3-(4-(2-(5-(hydroxymethyl)furan-2-yl)-6-(phenylsulfonyl)imidazo[4,5-d]pyrrolo[2,3-b]pyridin-1(6H)-yl)-1H-pyrazol-1-yl)propanenitrile (0.3 g, 0.6 mmol) in a mixture of 5 mL of tetrahydrofuran and 5 mL of methanol, then add 5 mL of 1 M sodium hydroxide. After stirring for 5 h at room temperature, the reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum. The residue was purified using a silica gel column (petroleum ether:ethyl acetate=1:1) to yield LXS46: 3-(4-(2-(5-(Hydroxymethyl)furan-2-yl)imidazo[4,5-d]pyrrolo[2,3-b]pyridin-1(6H)-yl)-1H-pyrazol-1-yl) propanenitrile (0.1 g, 46%). ¹ HNMR (300 MHz, DMSO-d₆): δ=11.96 (s, 1H), 8.79 (s, 1H), 8.06 (s, 1H), 7.94 (s, 1H), 7.59 (s, 1H), 7.02 (d, J=6.0 Hz, 1H), 6.78 (s, 1H), 6.59 (d, J=6.0 Hz, 1H), 5.04 (t, J=7.5 Hz, 2H), 4.39 (s, 2H), 3.28 (t, J=7.5 Hz, 2H) ppm; ¹³ C NMR (75 MHz, DMSO-d₆) δ 153.89, 151.18, 148.69, 142.17, 141.89, 130.74, 129.74, 129.04, 127.17, 120.73, 117.77, 115.63, 107.94, 104.05, 100.05, 99.49, 57.40, 49.27, 15.93 ppm; HRMS (ESI): m/z [M+H]⁺. C₁₉H₁₆N₇O₂ calculated value 374.1360, measured value 374.1377.

### Example 47

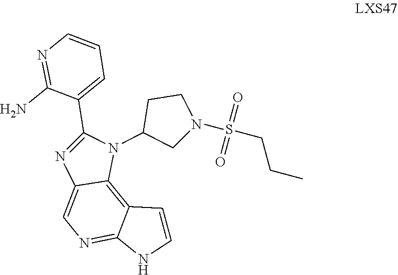 [SMILES: CCCS(=O)(=O)N1CCC(n2c(-c3cccnc3N)nc3cnc4[nH]ccc4c32)C1]

### 3-(1-(1-(propylsulfonyl)pyrrolidin-3-yl)-1,6-dihydroimidazo[4,5-d]pyrrolo[2,3-b]pyridin-2-yl)pyridin-2-amine

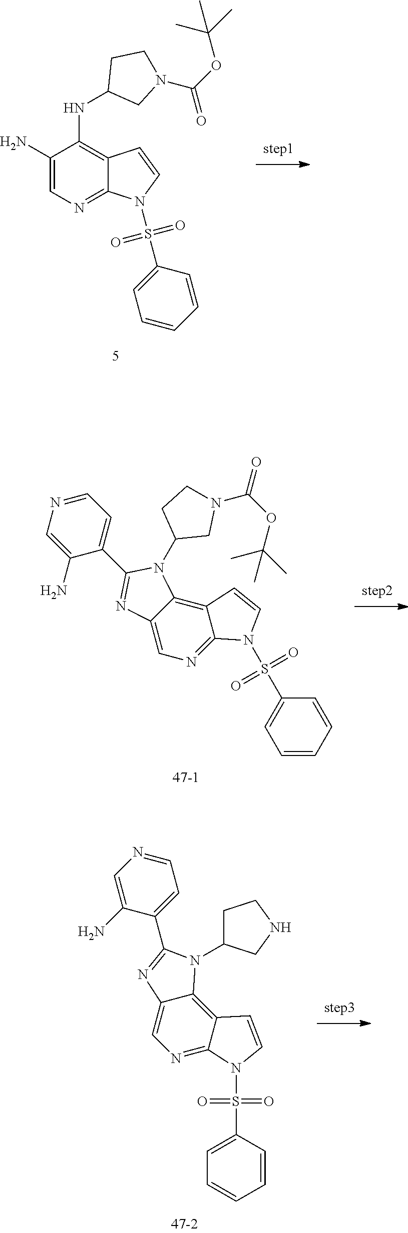 [SMILES: CC(C)(C)OC(=O)N1CCC(Nc2c(N)cnc3c2ccn3S(=O)(=O)c2ccccc2)C1 ; CC(C)(C)OC(=O)N1CCC(n2c(-c3ccncc3N)nc3cnc4c(ccn4S(=O)(=O)c4ccccc4)c32)C1 ; Nc1cnccc1-c1nc2cnc3c(ccn3S(=O)(=O)c3ccccc3)c2n1C1CCNC1]

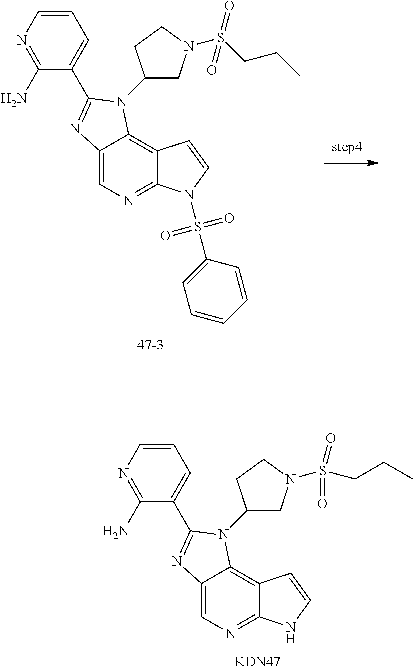

Step 1: Dissolve tert-butyl 3-(((5-amino-1-(phenylsulfonyl)-1H-pyrrolo[2,3-b]pyridin-4-yl)amino)pyrrolidine-1-carboxylate (0.3 g, 0.7 mmol) in 10 mL of DMF, add Na₂S₂O₅ (0.7 g, 3.5 mmol) followed by adding 2-amino-3-pyridinecarboxaldehyde (0.2 g, 1.4 mmol). Stir for 12 h at 90° C. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum. The residue was purified using a silica gel column (petroleum ether:ethyl acetate=1:1) to yield tert-butyl 3-(2-(2-aminopyridin-3-yl)-6-(phenylsulfonyl)imidazo[4,5-d]pyrrolo[2,3-b]pyridin-1(6H)-yl)pyrrolidine-1-carboxylate as a yellow solid (0.3 g, 82%). HRMS (ESI): m/z [M+H]⁺. C₂₈H₃₀N₇O₄S calculated value 560.2074, measured value 560.2077.

Step 2: Dissolve tert-butyl 3-(2-(2-aminopyridin-3-yl)-6-(phenylsulfonyl)imidazo[4,5-d]pyrrolo[2,3-b]pyridin-1(6H)-yl)pyrrolidine-1-carboxylate (0.3 g, 0.5 mmol) in 10 mL dichloromethane, slowly add trifluoroacetic acid (0.6 g, 5.0 mmol) and stir at room temperature for 12 hours. The product was concentrated under vacuum and 3-(6-(phenylsulfonyl)-1-(pyrrolidin-3-yl)-1,6-dihydroimidazo[4,5-d]pyrrolo[2,3-b]pyridin-2-yl)pyridin-2-amine was obtained as a light yellow oil (0.2 g, 81%). The product can be used directly in the next step without further purification. HRMS (EST): m/z [M+H]⁺. C₂₃H₂₂N₇O₂S calculated value 460.1550, measured value 460.1566.

Step 3: Dissolve 3-(6-(phenylsulfonyl)-1-(pyrrolidin-3-yl)-1,6-dihydroimidazo[4,5-d]pyrrolo[2,3-b]pyridin-2-yl)pyridin-2-amine (0.2 g, 0.4 mmol) in 10 mL of tetrahydrofuran, add DIPEA (0.1 g, 0.8 mmol) followed by the slow dropwise addition of propylsulfonyl chloride (0.1 g, 0.6 mmol) and then stir for 3 h at room temperature. Monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield 3-(6-(phenylsulfonyl)-1-(1-(propylsulfonyl)pyrrolidin-3-yl)-1,6-dihydroimidazo[4,5-d]pyrrolo[2,3-b]pyridin-2-yl) pyridin-2-amine as a pale yellow oil (0.2 g, 81%). The product can be used directly in the next step without further purification. HRMS (ESI): m/z [M+H]⁺. C₂₆H₂₈N₇O₄S₂ calculated value 566.1639, measured value 566.1645.

Step 4: 3-(6-(Phenylsulfonyl)-1-(1-(propylsulfonyl)pyrrolidin-3-yl)-1,6-dihydroimidazo[4,5-d]pyrrolo[2,3-b]pyridin-2-yl) pyridin-2-amine (0.2 g, 0.4 mmol) was dissolved in a solvent mixture of 5 mL tetrahydrofuran and 5 mL methanol, followed by adding 5 mL of 1 M sodium hydroxide. Stir the mixture at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS47: 3-(1-(1-(propylsulfonyl)pyrrolidin-3-yl)-1,6-dihydroimidazo[4,5-d]pyrrolo[2,3-]pyridin-2-yl)pyridin-2-amine (0.1 g, 67%). ¹ HNMR (300 MHz, DMSO-d₆): δ=12.06 (s, 1H), 8.83 (s, 1H), 8.03 (t, J=7.5 Hz, 1H), 7.74 (s, 2H), 7.66 (d, J=6.0 Hz, 1H), 7.56 (s, 1H), 6.81 (s, 1H), 6.68 (t, J=7.5 Hz, 1H), 3.65-3.79 (m, 1H), 3.12-3.36 (m, 2H), 3.10 (t, J=9.0 Hz, 2H), 2.70-2.81 (m, 2H), 1.85-2.14 (m, 2H), 1.69 (m, 2H), 0.93 (t, J=7.5 Hz, 3H) ppm; ¹³ C NMR (75 MHz, DMSO-d₆) δ 156.68, 153.77, 148.64, 146.69, 142.19, 134.84, 129.04, 127.16, 120.74, 118.74, 115.63, 113.59, 99.49, 60.47, 56.83, 56.18, 50.04, 26.25, 13.38, 12.48 ppm; HRMS (ESI): m/z [M+H]⁺. C₂₀H₂₄N₇O₂S calculated value 426.1707, measured value 426.1705.

### Example 48

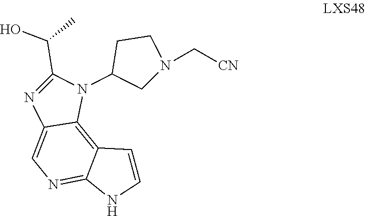 [SMILES: C[C@@H](O)c1nc2cnc3[nH]ccc3c2n1C1CCN(CC#N)C1]

### 2-(3-(2-((R)-1-Hydroxyethyl)imidazo[4,5-d]pyrrolo[2,3-b]pyridin-1(6H)-yl)pyrrolidin-1-yl)acetonitrile

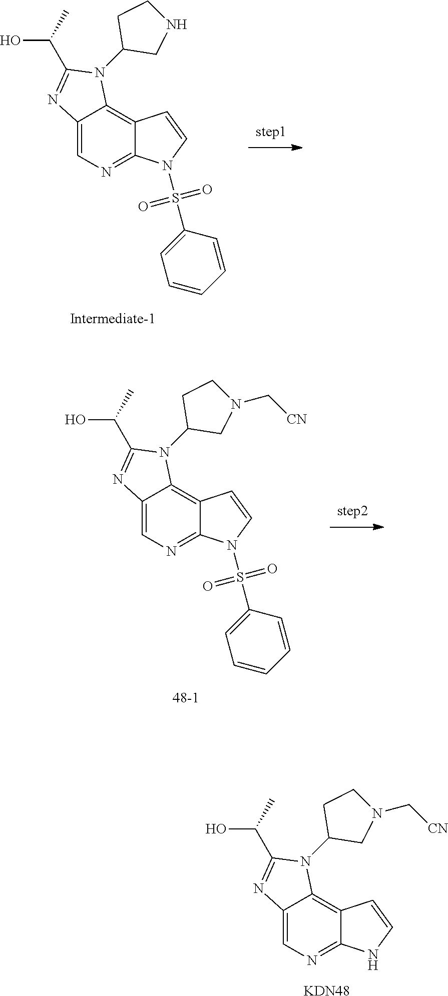

Step 1: Dissolve (1R)-1-(6-(phenylsulfonyl)-1-(pyrrolidin-3-yl)-1,6-dihydroimidazo[4,5-d]pyrrolo[2,3-b]pyridin-2-yl)ethanol (0.3 g, 0.7 mmol) in 10 mL of tetrahydrofuran, add DIPEA (0.2 g, 1.4 mmol) followed by the slow dropwise addition of 2-bromoacetonitrile (0.2 g, 1.1 mmol). Raise the temperature to maintain reflux and stir for 3 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield 2-(3-(2-((R)-1-hydroxyethyl)-6-(phenylsulfonyl)imidazo[4,5-d]pyrrolo[2,3-b]pyridin-1(6H)-yl)pyrrolidin-1-yl)acetonitrile as a pale yellow oil (0.2 g, 61%). The product can be used directly in the next step without further purification. HRMS (ESI): m/z [M+H]⁺. C₂₂H₂₃N₆O₃S calculated value 451.1547, measured value 451.1550.

Step 2: Dissolve 2-(3-(2-((R)-1-hydroxyethyl)-6-(phenylsulfonyl)imidazo[4,5-d]pyrrolo[2,3-b]pyridin-1(6H)-yl)pyrrolidin-1-yl)acetonitrile (0.2 g, 0.4 mmol) in a mixture of 5 mL of tetrahydrofuran and 5 mL of methanol, and stir at room temperature for 5 hours after adding 5 mL of 1 M sodium hydroxide. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate=1:1) to yield LXS48: 2-(3-(2-((R)-1-Hydroxyethyl)imidazo[4,5-d]pyrrolo[2,3-b]pyridin-1(6H)-yl)pyrrolidin-1-yl)acetonitrile (0.1 g, 73%). ¹ HNMR (300 MHz, DMSO-d₆): δ=11.86 (s, 1H), 8.87 (s, 1H), 7.59 (s, 1H), 6.89 (s, 1H), 4.53-4.68 (m, 1H), 3.68-3.78 (m, 1H), 3.48 (s, 2H), 2.56-2.81 (m, 2H), 2.20-2.30 (m, 2H), 1.90-2.15 (m, 2H), 1.93 (t, J=6.0 Hz, 3H) ppm; ¹³ C NMR (75 MHz, DMSO-d₆) δ 148.59, 142.17, 129.04, 127.16, 120.75, 115.63, 114.78, 99.28, 63.69, 58.18, 57.39, 54.83, 50.04, 26.97, 22.86 ppm; HRMS (ESI): m/z [M+H]⁺. C₁₆H₁₉N₆O calculated value 311.1615, measured value 311.1618.

### Example 49

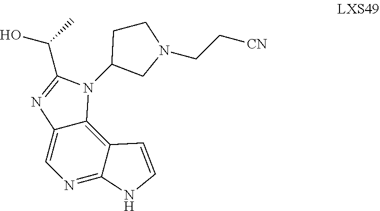 [SMILES: C[C@@H](O)c1nc2cnc3[nH]ccc3c2n1C1CCN(CCC#N)C1]

### 3-(3-(2-((R)-1-Hydroxyethyl)imidazo[4,5-d]pyrrolo[2,3-b]pyridin-1(6H)-yl)pyrrolidin-1-yl)propanenitrile

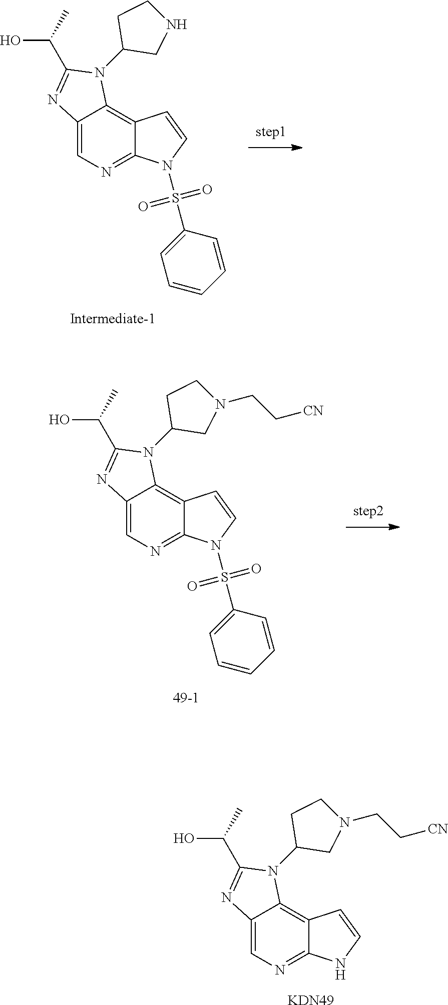

Step 1 Dissolve (1R)-1-(6-(phenylsulfonyl)-1-(pyrrolidin-3-yl)-1,6-dihydroimidazo[4,5-d]pyrrolo[2,3-b]pyridin-2-yl)ethanol (0.3 g, 0.7 mmol) in 10 mL of tetrahydrofuran, add DIPEA (0.2 g, 1.4 mmol) followed by the slow dropwise addition of 3-bromopropionitrile (0.2 g, 1.1 mmol). Raise the temperature to maintain reflux and stir for 3 h. The reaction was monitored by TLC for completion. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield 2-(3-(2-((R)-1-hydroxyethyl)-6-(phenylsulfonyl)imidazo[4,5-d]pyrrolo[2,3-b]pyridin-1(6H)-yl)pyrrolidin-2-yl)propanenitrile as a pale yellow oil (0.2 g, 59%). The product can be used directly in the next step without further purification. HRMS (ESI): m/z [M+H]⁺. C₂₃H₂₅N₆O₃S calculated value 465.1703, measured value 465.1709.

Step 2: Dissolve 2-(3-(2-((R)-1-hydroxyethyl)-6-(phenylsulfonyl)imidazo[4,5-d]pyrrolo[2,3-b]pyridin-1(6H)-yl)pyrrolidin-1-yl)propanenitrile (0.2 g, 0.4 mmol) in a mixture of 5 mL of tetrahydrofuran and 5 mL of methanol, and stir at room temperature for 5 hours after adding 5 mL of 1 M sodium hydroxide. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate=1:1) to yield LXS49: 3-(3-(2-((R)-1-Hydroxyethyl)imidazo[4,5-d]pyrrolo[2,3-b]pyridin-1(6H)-yl)pyrrolidin-1-yl)propanenitrile (0.1 g, 72%). ¹ HNMR (300 MHz, DMSO-d₆): δ=12.14 (s, 1H), 8.97 (s, 1H), 7.53 (s, 1H), 6.91 (s, 1H), 4.58-4.72 (m, 1H), 3.69-3.81 (m, 1H), 3.01 (t, J=9.0 Hz, 2H), 2.72-2.85 (m, 2H), 2.70 (t, J=9.0 Hz, 2H), 2.22-2.36 (m, 2H), 1.93-2.19 (m, 2H), 1.58 (t, J=7.5 Hz, 3H) ppm; ¹³ C NMR (75 MHz, DMSO-d₆) δ 148.66, 142.98, 129.03, 127.17, 120.77, 119.04, 115.69, 99.38, 63.69, 58.94, 57.69, 55.16, 27.64, 22.85, 17.17 ppm; HRMS (ESI): m/z [M+H]⁺. C₁₇H₂₁N₆O calculated value 325.1771, measured value 325.1777.

### Example 50

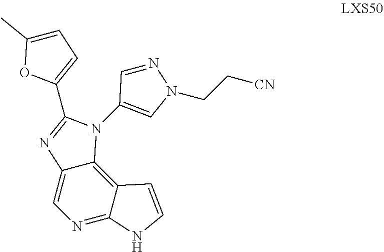 [SMILES: Cc1ccc(-c2nc3cnc4[nH]ccc4c3n2-c2cnn(CCC#N)c2)o1]

### 3-(4-(2-(5-Methylfuran-2-yl)imidazo[4,5-d]pyrrolo[2,3-b]pyridin-1(6H)-yl)-1H-pyrazol-1-yl)propanenitrile

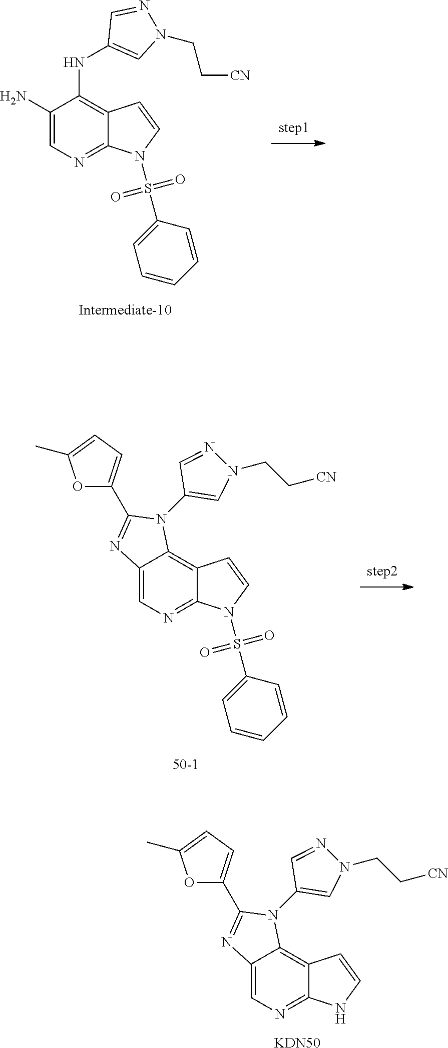

Step 1: Dissolve 3-(4-((5-amino-1-(phenylsulfonyl)-1H-pyrrolo[2,3-b]pyridin-4-yl)amino)-1H-pyrazol-1-yl)propanenitrile (0.3 g, 0.7 mmol) in 10 mL of DMF, add Na₂S₂O₅ (0.8 g, 4 mmol) followed by adding 5-methylfurfural (0.2 g, 1.4 mmol). Stir for 12 h at 90° C. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield 3-(4-(2-(5-methylfuran-2-yl)-6-(phenylsulfonyl)imidazo[4,5-d]pyrrolo[2,3-b]pyridin-1(6H)-yl)-1H-pyrazol-1-yl)propanenitrile as a yellow oil (0.3 g, 82%). HRMS (ESI): m/z [M+H]⁺. C₂₅H₂₀N₇O₃S calculated value 498.1343, measured value 498.1350.

Step 2: Dissolve 3-(4-(2-(5-methylfuran-2-yl)-6-(phenylsulfonyl)imidazo[4,5-d]pyrrolo[2,3-b]pyridin-1(6H)-yl)-1H-pyrazol-1-yl)propanenitrile (0.3 g, 0.6 mmol) in a solvent mixture of 5 mL of tetrahydrofuran and 5 mL of methanol, followed by adding 5 mL of 1 M sodium hydroxide. The mixture was then stirred at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate =1:1) to yield LXS50: 3-(4-(2-(5-methylfuran-2-yl)imidazo[4,5-d]pyrrolo[2,3-b]pyridin-1(6H)-yl)-1H-pyrazol-1-yl)propanenitrile (0.1 g, 46%). ¹ HNMR (300 MHz, DMSO-d₆): δ=11.87 (s, 1H), 8.65 (s, 11H), 8.48 (s, 1H), 7.95 (s, 1H), 7.34 (s, 1H), 6.32 (d, J=3.0 Hz, 1H), 6.21 (s, 1H), 5.95 (s, 1H), 4.61 (t, J=4.5 Hz, 2H), 3.26 (t, J=6.0 Hz, 2H), 2.36 (s, 3H) ppm; ¹³ C NMR (75 MHz, DMSO-d₆) δ 154.31, 145.68, 143.29, 142.72, 138.24, 135.85, 135.54, 134.05, 129.75, 124.57, 118.82, 118.75, 113.43, 108.51, 104.57, 96.45, 48.05, 19.20, 13.73 ppm; HRMS (ESI): m/z [M+H]⁺. C₁₉H₁₆N₇O calculated value 358.1411, measured value 358.1414.

### Example 51

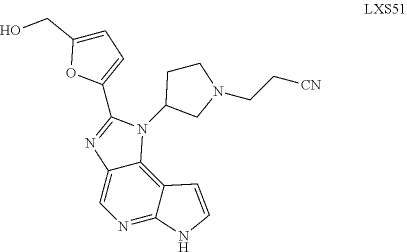 [SMILES: N#CCCN1CCC(n2c(-c3ccc(CO)o3)nc3cnc4[nH]ccc4c32)C1]

### 3-(3-(2-(5-(Hydroxymethyl)furan-2-yl)imidazo[4,5-d]pyrrolo[2,3-b]pyridin-1(6H)-yl)pyrrolidin-1-yl)propanenitrile

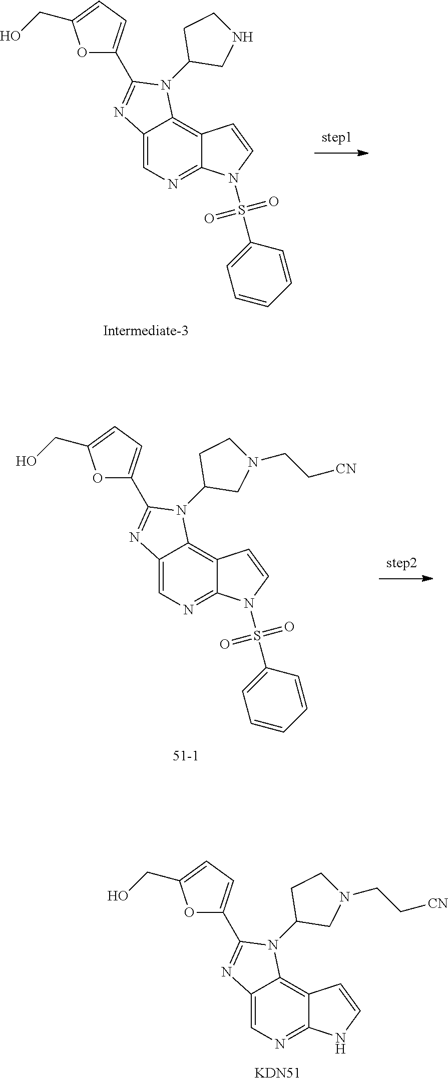

Step 1: Dissolve (5-(6-(phenylsulfonyl)-1-(pyrrolidin-3-yl)-1,6-dihydroimidazo[4,5-d]pyrrolo[2,3-b]pyridin-2-yl)furan-2-methanol (0.3 g, 0.6 mmol) in 10 mL of tetrahydrofuran, add DIPEA (0.2 g, 1.2 mmol) followed by the slow dropwise addition of 3-bromopropionitrile (0.2 g, 0.9 mmol). Raise the temperature to maintain reflux and stir for 3 h. The reaction was monitored by TLC for completion. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield 3-(3-(2-(5-(hydroxymethyl)furan-2-yl)-6-(phenylsulfonyl)imidazo[4,5-d]pyrrolo[2,3-b]pyridin-1(6H)-yl)pyrrolidin-1-yl)propanenitrile as a pale yellow oil (0.2 g, 60%). The product can be used directly in the next step without further purification. HRMS (ESI): m/z [M+H]⁺. C₂₆H₂₅N₆O₄S calculated value 517.1653, measured value 517.1659.

Step 2: Dissolve 3-(3-(2-(5-(hydroxymethyl)furan-2-yl)-6-(phenylsulfonyl)imidazo[4,5-d]pyrrolo[2,3-b]pyridin-1(6H)-yl)pyrrolidin-1-yl)propanenitrile (0.2 g, 0.4 mmol) in a solvent mixture of 5 mL tetrahydrofuran and 5 mL methanol, followed by adding 5 mL of 1 M sodium hydroxide. Stir for 5 h at room temperature and monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate=1:1) to yield LXS51: 3-(3-(2-(5-(Hydroxymethyl)furan-2-yl)imidazo[4,5-d]pyrrolo[2,3-b]pyridin-1(6H)-yl)pyrrolidin-1-yl)propanenitrile (0.1 g, 69%). ¹ HNMR (300 MHz, DMSO-d₆); δ=12.03 (s, 1H), 8.88 (s, 1H), 7.59 (s, 1H), 7.02 (d, J=9.0 Hz, 1H), 6.84 (s, 1H), 6.59 (d, J=9.0 Hz, 1H), 4.39 (s, 2H), 3.64-3.78 (m, 1H), 3.03 (t, J=9.0 Hz, 2H), 2.74-2.86 (m, 2H), 2.73 (t, J=9.0 Hz, 2H), 2.20-2.35 (m, 2H), 1.90-2.15 (m, 2H) ppm; ¹³ CNMR (75 MHz, DMSO-d₆) δ 153.88, 151.18, 148.64, 144.94, 142.17, 129.04, 127.18, 120.79, 119.04, 115.63, 107.94, 104.06, 99.38, 57.39, 57.01, 55.84, 55.19, 27.05, 17.16 ppm, HRMS (ESI): m/z [M+H]⁺. C₂₀H₂₁N₆O₂ calculated value 377.1721, measured value 377.1728.

### Example 52

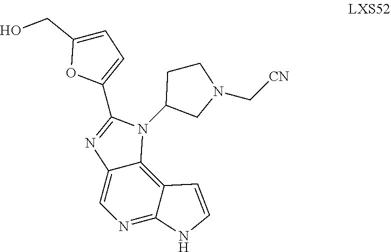 [SMILES: N#CCN1CCC(n2c(-c3ccc(CO)o3)nc3cnc4[nH]ccc4c32)C1]

### 2-(3-(2-(5-(hydroxymethyl)furan-2-yl)imidazo[4,5-d]pyrrolo[2,3-b]pyridin-1(6H)-yl)pyrrolidin-1-yl)acetonitrile

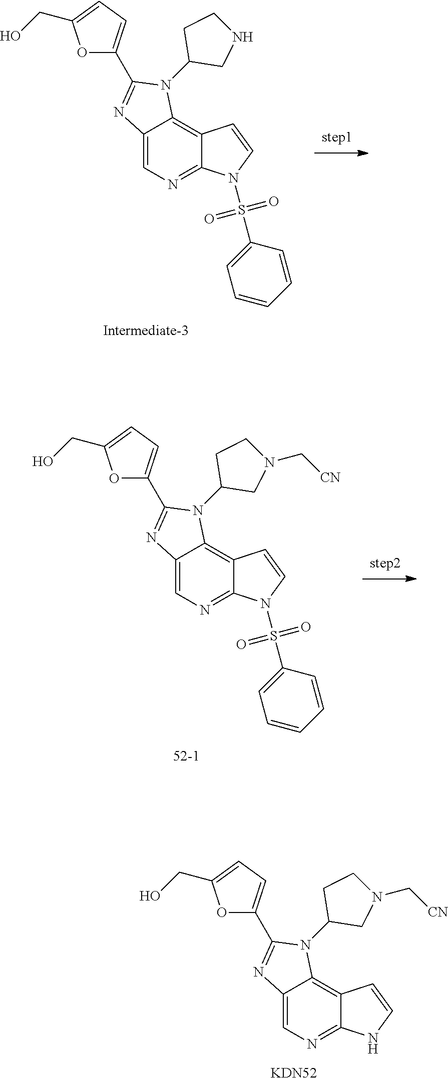

Step 1: Dissolve (5-(6-(phenylsulfonyl)-1-(pyrrolidin-3-yl)-1,6-dihydroimidazo[4,5-d]pyrrolo[2,3-b]pyridin-2-yl)furan-2-methanol (0.3 g, 0.6 mmol) in 10 mL of tetrahydrofuran, add DIPEA (0.2 g, 1.2 mmol) followed by the slow dropwise addition of 2-bromoacetonitrile (0.2 g, 0.9 mmol). Raise the temperature to maintain reflux and stir for 3 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield 3-(3-(2-(5-(hydroxymethyl)furan-2-yl)-6-(phenylsulfonyl)imidazo[4,5-d]pyrrolo[2,3-b]pyridin-1(6H)-yl)pyrrolidin-1-yl) acetonitrile as a pale yellow oil (0.2 g, 61%). The product can be used directly in the next step without further purification. HRMS (ESI): m/z [M+H]⁺. C₂₅H₂₃N₆O₄S calculated value 503.1496, measured value 503.1500.

Step 2: Dissolve 3-(3-(2-(5-(hydroxymethyl)furan-2-yl)-6-(phenylsulfonyl)imidazo[4,5-d]pyrrolo[2,3-b]pyridin-1(6H)-yl)pyrrolidin-1-yl)acetonitrile (0.2 g, 0.4 mmol) in a solvent mixture of 5 mL of tetrahydrofuran and 5 mL of methanol, followed by adding 5 mL of 1 M sodium hydroxide. Stir for 5 h at room temperature and monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate=1:1) to yield LXSS2: 2-(3-(2-(5-(hydroxymethyl)furan-2-yl)imidazo[4,5-d]pyrrolo[2,3-b]pyridin-1(6H)-yl)pyrrolidin-1-yl)acetonitrile (0.1 g, 69%). ¹ HNMR (300 MHz, DMSO-d₆): δ=11.99 (s, 1H), 8.67 (s, 1H), 7.58 (s, 1H), 7.06 (d, J=7.5 Hz, 1H), 6.88 (s, 1H), 6.54 (d, J=7.5 Hz, 1H), 4.33 (s, 2H), 3.69-3.83 (m, 1H), 3.48 (s, 2H), 2.56-2.81 (m, 2H), 2.20-2.30 (m, 2H), 1.93-2.18 (m, 2H) ppm; ¹³CNMR (75 MHz, DMSO-d₆) δ 153.97, 151.19, 148.63, 144.95, 142.18, 129.03, 127.17, 120.75, 115.69, 114.84, 107.94, 104.05, 99.39, 57.36, 56.72, 55.09, 54.81, 50.05, 26.29 ppm; HRMS (EST): m/z [M+H]⁺. C₁₉H₁₉N₆O₂ calculated value 363.1564, Measured value 363.1569.

### Example 53

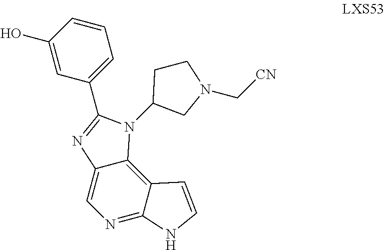 [SMILES: N#CCN1CCC(n2c(-c3cccc(O)c3)nc3cnc4[nH]ccc4c32)C1]

### 2-(3-(2-(3-Hydroxyphenyl)imidazo[4,5-d]pyrrolo[2,3-b]pyridin-1(6H)-yl)pyrrolidin-1-yl)acetonitrile

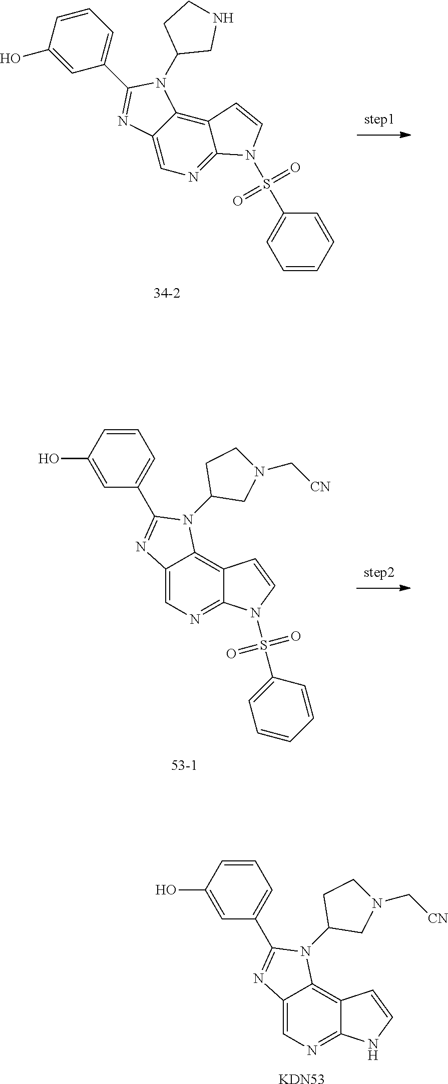

Step 1: Dissolve 3-(6-(phenylsulfonyl)-1-(pyrrolidin-3-yl)-1,6-dihydroimidazo[4,5-d]pyrrolo[2,3-b]pyridin-2-yl)phenol (0.3 g, 0.7 mmol) in 10 mL of tetrahydrofuran, add DIPEA (0.2 g, 1.4 mmol) followed by slow dropwise addition of 2-bromoacetonitrile (0.2 g, 1.1 mmol). Raise the temperature to maintain reflux and stir for 3 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield 2-(3-(2-(3-hydroxyphenyl)-6-(phenylsulfonyl)imidazo[4,5-d]pyrrolo[2,3-b]pyridin-1(6H)-yl)pyrrolidin-1-yl) acetonitrile as a yellow oil (0.2 g, 61%). The product can be used directly in the next step without further purification. HRMS (EST): m/z [M+H]⁺. C₂₆H₂₃N₆O₃S calculated value 499.1547, measured value 499.1550.

Step 2: Dissolve (3-(2-(3-hydroxyphenyl)-6-(phenylsulfonyl)imidazo[4,5-d]pyrrolo[2,3-b]pyridin-1(6H)-yl)pyrrolidin-1-yl)acetonitrile (0.2 g, 0.4 mmol) in a solvent mixture of 5 mL tetrahydrofuran and 5 mL methanol, add 1 M sodium hydroxide 5 mL and stir at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether; ethyl acetate=1:1) to yield LXS53: 2-(3-(2-(3-Hydroxyphenyl)imidazo[4,5-d]pyrrolo[2,3-b]pyridin-1(6H)-yl)pyrrolidin-1-yl)acetonitrile (0.1 g, 70%). ¹HNMR (300 MHz, DMSO-d₆): δ=12.09 (s, 1H), 8.83 (s, 1H), 7.84 (d, J=6.0 Hz, 1H), 7.66 (s, 1H), 7.34 (t, J=6.0 Hz, 1H), 7.04 (s, 1H), 6.91 (d, J=6.0 Hz, 1H), 6.83 (s, 1H), 3.71-3.82 (m, 1H), 3.49 (s, 2H), 2.58-2.83 (m, 2H), 2.17-2.36 (m, 2H), 1.90-2.16 (m, 2H) ppm; ^{—}CNMR (75 MHz, DMSO-d₆) δ 157.59, 153.75, 148.63, 142.17, 132.05, 130.65, 129.05, 127.16, 120.75, 120.11, 115.94, 115.65, 114.85, 112.94, 99.38, 57.3, 54.84, 50.04, 26.84 ppm; HRMS (EST): m/z [M+H]⁺. C₂₀H₁₉N₆O calculated value 359.1615, measured value 359.1619.

### Example 54

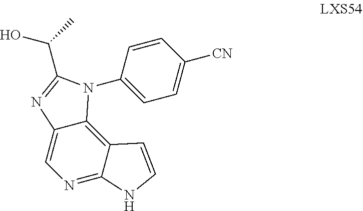 [SMILES: C[C@@H](O)c1nc2cnc3[nH]ccc3c2n1-c1ccc(C#N)cc1]

### (R)-4-(2-(1-Hydroxyethyl)imidazo[4,5-d]pyrrolo[2,3-b]pyridin-1(6H)-yl)benzonitrile

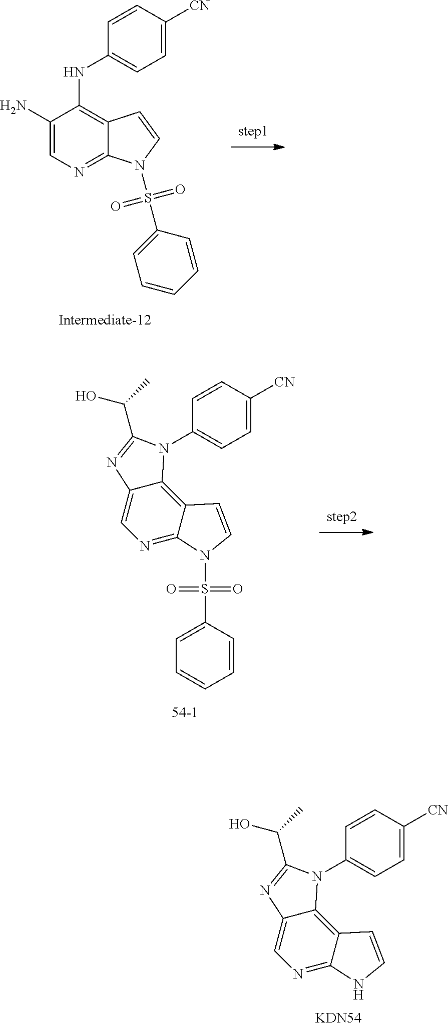 [SMILES: N#Cc1ccc(Nc2c(N)cnc3c2ccn3S(=O)(=O)c2ccccc2)cc1 ; C[C@@H](O)c1nc2cnc3c(ccn3S(=O)(=O)c3ccccc3)c2n1-c1ccc(C#N)cc1 ; C[C@@H](O)c1nc2cnc3[nH]ccc3c2n1-c1ccc(C#N)cc1 ; "KDN54"]

Step 1: Triethyloxonium tetrafluoroborate (0.5 g, 2.4 mmol) and (R)-lactamide (0.2 g, 2.4 mmol) were dissolved in 10 mL of tetrahydrofuran, stirred at room temperature for 3 h and concentrated under vacuum to obtain the mixture as an oil, followed by the addition of 10 mL of ethanol to dissolve and the addition of 4-((5-amino-1-(phenylsulfonyl)-1H-pyrrolo[2,3-b]pyridin-4-yl)amino)benzonitrile (0.3 g, 0.8 mmol). Then, raise the temperature while maintaining reflux and stir for 3 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield (R)-4-(2-(1-hydroxyethyl)-6-(phenylsulfonyl)imidazo[4,5-d]pyrrolo[2,3-b]pyridin-1(6H)-yl)benzonitrile as a light green oil (0.3 g, 88%). HRMS (ESI): m/z [M+H]⁺. C₂₃H₁₈N₅O₃S calculated value 444.1125, measured value 444.1129.

Step 2: Dissolve (R)-4-(2-(1-hydroxyethyl)-6-(phenylsulfonyl)imidazo[4,5-d]pyrrolo[2,3-b]pyridin-1(6H)-yl)benzonitrile (0.3 g, 0.7 mmol) in a solvent mixture of 5 mL tetrahydrofuran and 5 mL methanol, add 1 M sodium hydroxide 5 mL and stir at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS54: (R)-4-(2-(1-Hydroxyethyl)imidazo[4,5-d]pyrrolo[2,3-b]pyridin-1(6H)-yl)benzonitrile (0.1 g, 49%). ¹HNMR (300 MHz, DMSO-d₆): δ=12.01 (s, 1H), 8.91 (s, 1H), 7.80 (d, J=9.0 Hz, 2H), 7.64 (d, J=9.0 Hz, 2H), 7.56 (s, 1H), 6.88 (s, 1H), 3.98-4.68 (m, 1H), 1.49 (d, J=7.5 Hz, 3H) ppm; ¹³ CNMR (75 MHz, DMSO-d₆) δ 151.75, 148.69, 142.16, 140.48, 134.27, 129.04, 127.18, 122.84, 120.73, 118.64, 115.67, 112.17, 99.38, 62.95, 22.84 ppm; HRMS(ESI): m/z [M+H]⁺. C₁₇H₁₄N₅O calculated value 304.1193, measured value 304.1203.

### Example 55

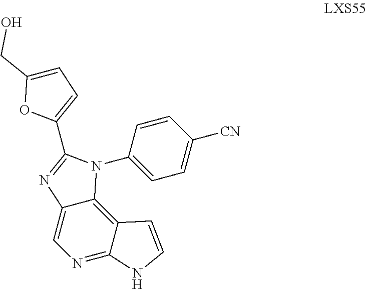 [SMILES: N#Cc1ccc(-n2c(-c3ccc(CO)o3)nc3cnc4[nH]ccc4c32)cc1]

### 4-(2-(5-(Hydroxymethyl)furan-2-yl)imidazo[4,5-d]pyrrolo[2,3-b]pyridin-1(6H)-yl)benzonitrile

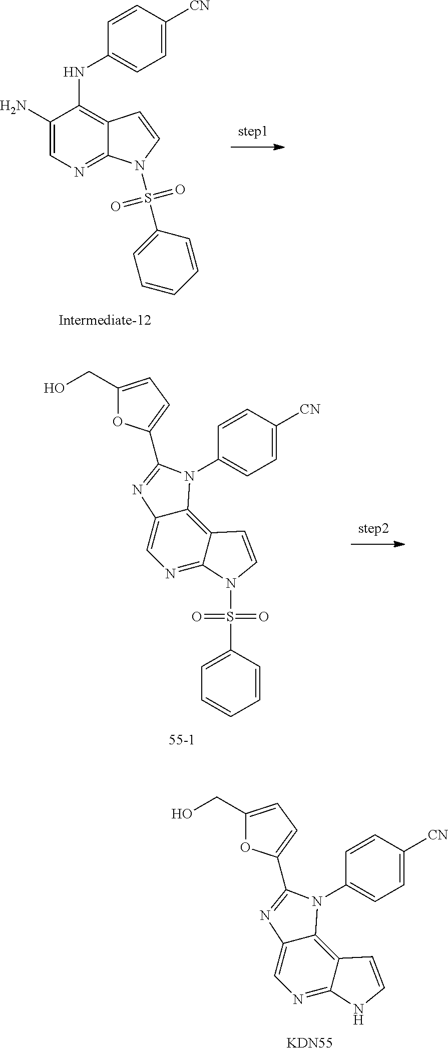

Step 1: Dissolve 4-((5-amino-1-(phenylsulfonyl)-1H-pyrrolo[2,3-b]pyridin-4-yl)amino)benzonitrile (0.3 g, 0.8 mmol) in 10 mL of DMF, add Na₂S₂O₅ (0.8 g, 4 mmol) followed by the addition of 5-hydroxymethylfurfural (0.2 g, 1.6 mmol) dropwise, then raise the temperature to 90° C. and stir for 12 h. The reaction was monitored for completion by TLC. Saturated sodium bicarbonate was added until the reaction solution was weakly basic, and the organic phase was separated. The aqueous phase was extracted twice with dichloromethane. The organic phases were combined and washed with saturated saline, dried over anhydrous sodium sulfate and concentrated under vacuum to yield 4-(2-(5-(hydroxymethyl)furan-2-yl)-6-(phenylsulfonyl)imidazo[4,5-d]pyrrolo[2,3-b]pyridin-1(6H)-yl)benzonitrile as a yellow oil (0.3 g, 79%). HRMS(ESI): m/z [M+H]⁺. C₂₆H₁₅N₅O₄S calculated value 496.1074, measured value 496.1080.

Step 2: Dissolve 4-(2-(5-(hydroxymethyl)furan-2-yl)-6-(phenylsulfonyl)imidazo[4,5-d]pyrrolo[2,3-b]pyridin-1(6H)-yl)benzonitrile (0.3 g, 0.6 mmol) in a solvent mixture of 5 mL tetrahydrofuran and 5 mL methanol, add 5 mL of 1 M sodium hydroxide and then stir at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum. The residue was purified using a silica gel column (petroleum ether:ethyl acetate=1:1) to yield LXSS5: 4-(2-(5-(hydroxymethyl)furan-2-yl)imidazo[4,5-d]pyrrolo[2,3-b]pyridin-1(6H)-yl)benzonitrile (0.1 g, 47%). ¹HNMR (300 MHz, DMSO-d₆): δ 11.91 (s, 1H), 8.73 (s, 1H), 7.83 (d, J=6.0 Hz, 2H), 7.68 (d, J=6.0 Hz, 2H), 7.56 (s, 1H), 7.02 (d, J=9.0 Hz, 1H), 6.83 (s, 1H), 6.59 (d, J=9.0 Hz, 1H), 4.39 (s, 2H) ppm; ¹³CNMR (75 MHz, DMSO-d₆) δ153.85, 151.17, 148.64, 142.59, 142.31, 134.27, 129.05, 127.17, 122.85, 120.73, 118.65, 115.69, 112.15, 107.94, 104.06, 99.38, 57.39 ppm; HRMS (ESI): m/z [M+H]⁺. C₂₀H₁₄N₅O₂ calculated value 356.1142, measured value 356.1149.

### Example 56

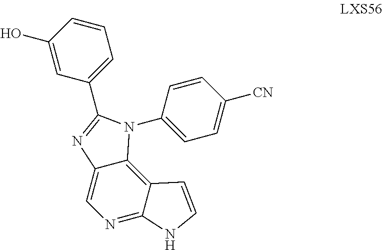 [SMILES: N#Cc1ccc(-n2c(-c3cccc(O)c3)nc3cnc4[nH]ccc4c32)cc1]

### 4-(2-(3-Hydroxyphenyl)imidazo[4,5-d]pyrrolo[2,3-b]pyridin-1(6H)-yl)benzonitrile

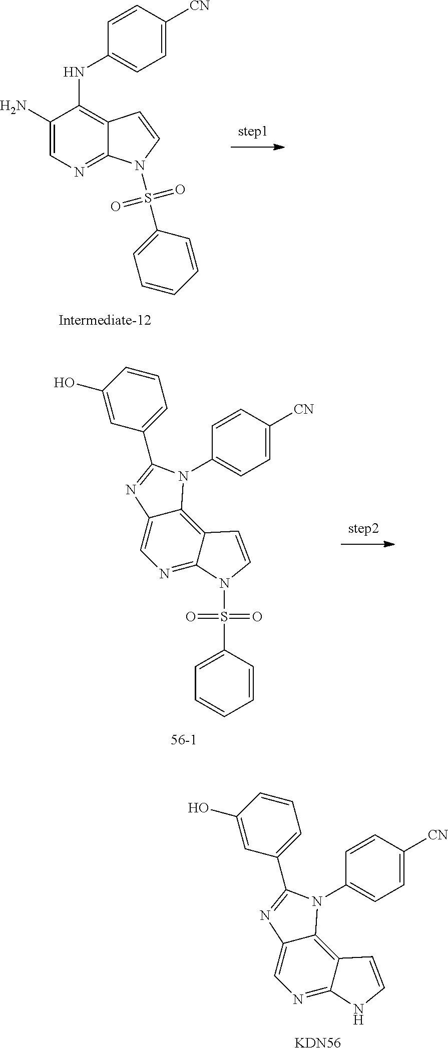

Step 1: Dissolve 4-((5-amino-1-(phenylsulfonyl)-1H-pyrrolo[2,3-b]pyridin-4-yl)amino)benzonitrile (0.3 g, 0.8 mmol) in 10 mL of DMF, add Na₂S₂O₅ (0.8 g, 4 mmol) followed by the addition of 3-hydroxybenzaldehyde (0.2 g, 1.6 mmol) dropwise, raise the temperature to 90° C. and stir for 12 h. The reaction was monitored for completion by TLC. Saturated sodium bicarbonate was added until the reaction solution was weakly basic, and the organic phase was separated. The aqueous phase was extracted twice with dichloromethane. The organic phases were combined and washed with saturated saline, dried over anhydrous sodium sulfate and concentrated under vacuum to yield 4-(2-(3-hydroxyphenyl)-6-(phenylsulfonyl)imidazo[4,5-d]pyrrolo[2,3-b]pyridin-1(6H)-yl)benzonitrile as a yellow oil (0.3 g, 79%). HRMS(ESI): m/z [M+H]⁺. C₂₇H₁₈N₅O₃S calculated value 492.1125, measured value 492.1134.

Step 2: 4-(2-(3-Hydroxyphenyl)-6-(phenylsulfonyl)imidazo[4,5-d]pyrrolo[2,3-b]pyridin-1(6H)-yl)benzonitrile (0.3 g, 0.6 mmol) was dissolved in a solvent mixture of 5 mL tetrahydrofuran and 5 mL methanol, and the mixture was stirred at room temperature for 5 h after the addition of 5 mL 1 M sodium hydroxide. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXSS6: 4-(2-(3-hydroxyphenyl)imidazo[4,5-d]pvrrolo[2,3-b]pyridin-1(6H)-yl)benzonitrile (0.1 g, 47%). ¹HNMR (300 MHz, DMSO-d₆): δ=12.06 (s, 1H), 8.83 (s, 1H), 7.84-7.93 (m, 1H), 7.81 (d, J=7.5 Hz, 2H), 7.64 (d, J=7.5 Hz, 2H), 7.58 (s, 1H), 7.34 (t, J=9.0 Hz, 1H), 7.08 (s, 1H), 6.91 (d, J=9.0 Hz, 1H), 6.88 (s, 1H) ppm; ¹³CNMR (75 MHz, DMSO-d₆) δ 157.89, 148.99, 144.47, 142.59, 142.16, 134.27, 132.04, 130.64, 129.05, 127.49, 122.84, 120.75, 120.16, 118.69, 115.96, 115.61, 112.94, 112.15, 99.54 ppm; HRMS (ESI): m/z [M+H]⁺. C₂₁H₁₄N₅O calculated value 352.1193, measured value 352.1199.

### Example 57

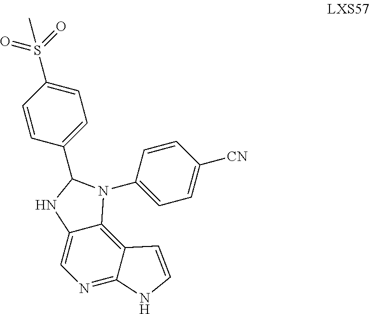 [SMILES: CS(=O)(=O)c1ccc(C2Nc3cnc4[nH]ccc4c3N2c2ccc(C#N)cc2)cc1]

### 4-(2-(4-(Methylsulfonyl)phenyl)-2,3-dihydroimidazo[4,5-d]pyrrolo[2,3-b]pyridin-1(6H)-yl)benzonitrile

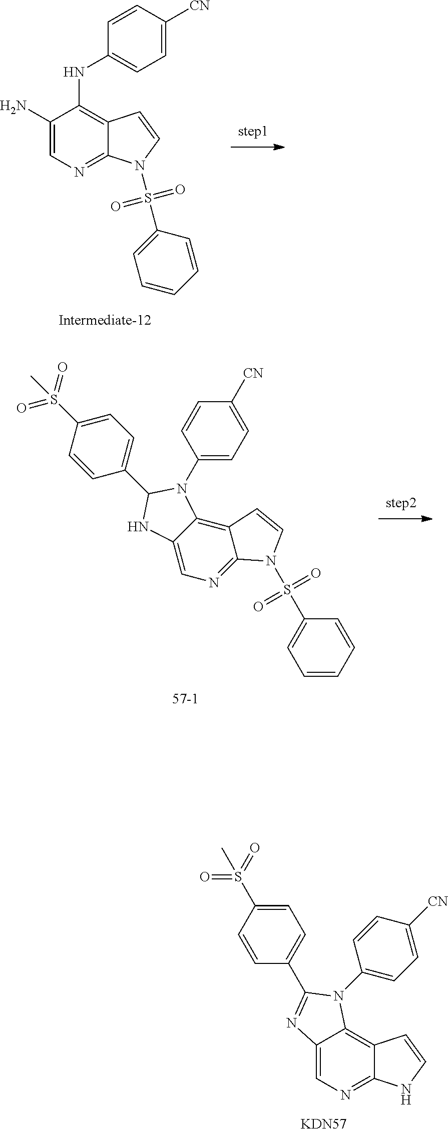

Step 1: Dissolve 4-((5-amino-1-(phenylsulfonyl)-1H-pyrrolo[2,3-b]pyridin-4-yl)amino)benzonitrile (0.3 g, 0.8 mmol) in 10 mL of DMF, add Na₂S₂O₅ (0.8 g, 4 mmol) followed by adding 4-methylsulfonyl benzaldehyde (0.3 g, 1.6 mmol) dropwise, then raise the temperature to 90° C. and stir for 12 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield 4-(2-(4-(methylsulfonyl)phenyl)-6-(phenylsulfonyl)-2,3-dihydroimidazo[4,5-d]pyrrolo[2,3-b]pyridin-1(6H)-yl)benzonitrile as a yellow oil (0.3 g, 70%). HRMS (ESI): m/z [M+H]⁺. C₂₈H₂₂N₅O₄S₂ calculated value 556.1108, measured value 556.1113.

Step 2: Dissolve 4-(2-(4-(methylsulfonyl)phenyl)-6-(phenylsulfonyl)-2,3-dihydroimidazo[4,5-d]pyrrolo[2,3-b]pyridin-1(6H)-yl)benzonitrile (0.3 g, 0.5 mmol) in a mixture of 5 mL of tetrahydrofuran and 5 mL of methanol, add 5 mL of 1 M sodium hydroxide and then stir at room temperature for 5 hours. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS57: 4-(2-(4-(methylsulfonyl)phenyl)-2,3-dihydroimidazo[4,5-d]pyrrolo[2,3-b]pyridin-1(6H)-yl)benzonitrile (0.1 g, 45%). ¹HNMR (300 MHz, DMSO-d₆): δ=11.86 (s, 1H), 7.89 (s, 1H), 7.74 (d, J=6.0 Hz, 2H), 7.63 (s, 1H), 7.54 (d, J=6.0 Hz, 2H), 7.42 (d, J=9.0 Hz, 2H), 6.88 (s, 1H), 6.78 (d, J=9.0 Hz, 2H), 5.08 (s, 1H), 3.32 (s, 1H) ppm; ¹³CNMR (75 MHz, DMSO-d₆) δ 153.77, 149.47, 139.04, 138.79, 134.41, 133.05, 131.18, 128.15, 127.93, 127.16, 123.38, 118.66, 114.28, 108.15, 101.04, 99.37, 87.84, 47.72 ppm; HRMS (ESI): m/z [M+H]⁺. C₂₂H₁₈N₅O₂S calculated value 416.1176, measured value 416.1180.

### Example 58

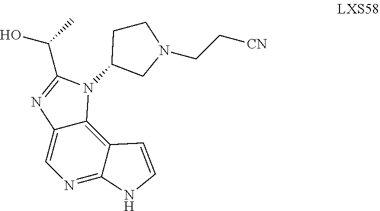 [SMILES: C[C@@H](O)c1nc2cnc3[nH]ccc3c2n1[C@@H]1CCN(CCC#N)C1]

### 3-((R)-3-(2-((R)-1-Hydroxyethyl)imidazo[4,5-d]pyrrolo[2,3-b]pyridin-1(6H)-yl)pyrrolidin-1-yl)propanenitrile

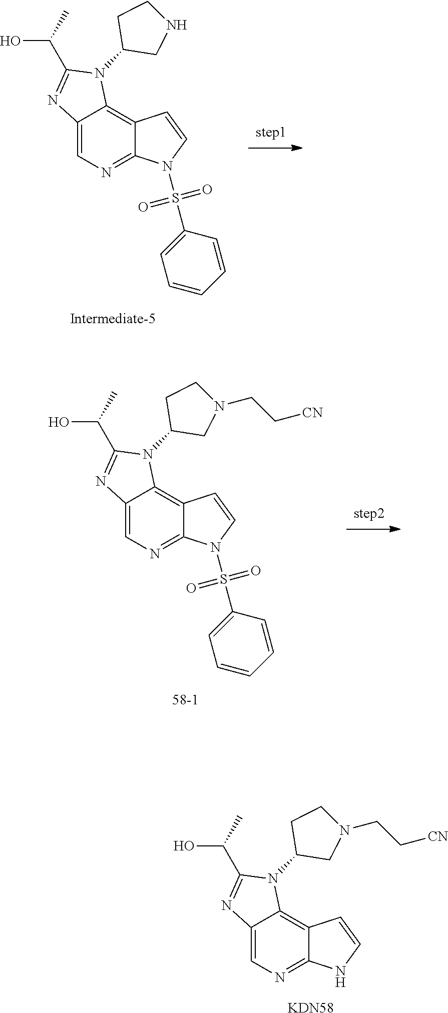

Step 1: Dissolve (R)-1-(6-(phenylsulfonyl)-1-((R-pyrrolidin-3-yl)-1,6-dihydroimidazo[4,5-d]pyrrolo[2,3-b]pyridin-2-yl)ethanol (0.3 g, 0.7 mmol) in 10 mL of tetrahydrofuran, add DIPEA (0.2 g, 1.4 mmol) and then slowly add 3-bromopropanenitrile (0.2 g, 1.1 mmol) dropwise. Raise the temperature to maintain reflux and stir for 3 h. Monitor the reaction for completion by TLC. Saturated sodium bicarbonate was added until the reaction solution was weakly basic, and the organic phase was separated. The aqueous phase was extracted twice using dichloromethane. The organic phases were then combined and washed with saturated saline, dried over anhydrous sodium sulfate and concentrated under vacuum to yield 3-((R)-3-(2-((R)-1-hydroxyethyl)-6-(phenylsulfonyl)imidazo[4,5-d]pyrrolo[2,3-b]pyridin-1(6H)-yl)pyrrolidin-1-yl propanenitrile as a pale yellow oil (0.2 g, 59%). The product can be used directly in the next step without further purification. HRMS (ESI): m/z [M+H]⁺. C₂₃H₂₅N₆O₃S calculated value 465.1703, measured value 465.1709.

Step 2: 3-((R)-3-(2-((R)-1-Hydroxyethyl)-6-(phenylsulfonyl)imidazo[4,5-d]pyrrolo[2,3-b]pyridin-1(6H)-yl)pyrrolidin-1-yl) propanenitrile (0.2 g, 0.4 mmol) was dissolved in a mixture of 5 mL tetrahydrofuran and 5 mL methanol, followed by adding 5 mL of 1 M sodium hydroxide. Stir the mixture at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate=1:1) to yield LXS58: 3-((R)-3-(2-((R)—I-Hydroxyethyl)imidazo[4,5-d]pyrrolo[2,3-b]pyridin-1(6H)-yl)pyrrolidin-1-yl)propanenitrile (0.1 g, 72%). ¹HNMR (300 MHz, DMSO-d₆): δ=11.98 (s, 1H), 8.73 (s, 1H), 7.56 (s, 1H), 6.78 (s, 1H), 4.61-4.75 (m, 1H), 3.65-3.83 (m, 1H), 3.04 (t, J=6.0 Hz, 2H), 2.70-2.83 (m, 2H), 2.73 (t, J=6.0 Hz, 2H), 2.20-2.31 (m, 2H), 1.90-2.15 (m, 2H), 1.49 (t, J=7.5 Hz, 3H) ppm; ¹³CNMR (75 MHz, DMSO-d₆) δ 148.64, 148.59, 142.19, 129.03, 127.16, 120.74, 119.04, 115.69, 99.39, 63.62, 58.91, 57.63, 55.16, 27.63, 22.84, 17.19 ppm; HRMS (ESI): m/z [M+H]⁺. C₁₇H₂₁N₆O calculated value 325.1771, measured value 325.1777.

### Example 59

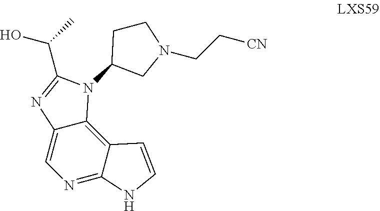 [SMILES: C[C@@H](O)c1nc2cnc3[nH]ccc3c2n1[C@H]1CCN(CCC#N)C1]

### 3-((S)-3-(2-((R)-1-hydroxyethyl)imidazo[4,5-d]pyrrolo[2,3-b]pyridin-1(6H)-yl)pyrrolidin-1-yl)propanenitrile

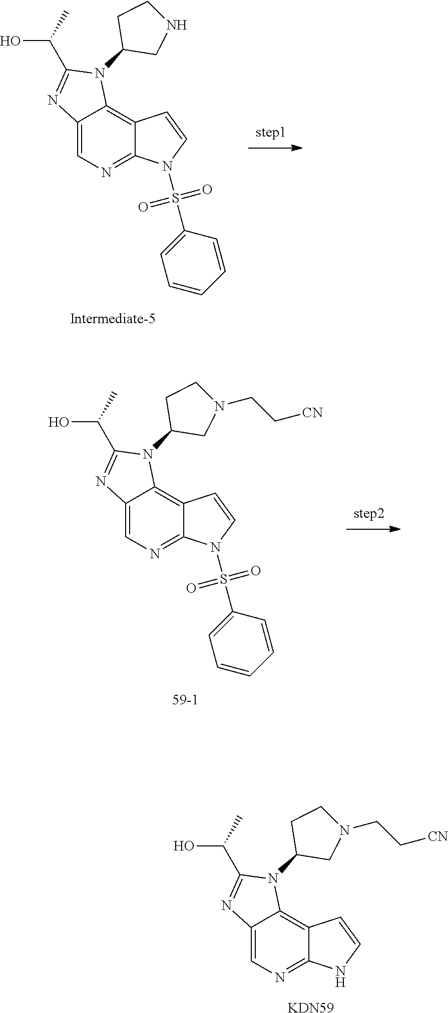

Step 1: Dissolve (R)-1-(6-(phenylsulfonyl)-1-((S-pyrrolidin-3-yl)-1,6-dihydroimidazo[4,5-d]pyrrolo[2,3-b]pyridin-2-yl)ethanol (0.3 g, 0.7 mmol) in 10 mL of tetrahydrofuran, add DIPEA (0.2 g, 1.4 mmol) and then slowly add 3-bromopropanenitrile (0.2 g, 1.1 mmol) dropwise. Raise the temperature to maintain reflux and stir for 3 h. Monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield 3-((S)-3-(2-((R)-1-hydroxyethyl)-6-(phenylsulfonyl)imidazo[4,5-d]pyrrolo[2,3-b]pyridin-1(6H)-yl)pyrrolidin-1-yl propanenitrile as a pale yellow oil (0.2 g, 59%). The product can be used directly in the next step without further purification. HRMS (ESI): m/z [M+H]⁺. C₂₃H₂₅N₆O₃S calculated value 465.1703, measured value 465.1711.

Step 2: 3-((S)-3-(2-((R)-1-Hydroxyethyl)-6-(phenylsulfonyl)imidazo[4,5-d]pyrrolo[2,3-b]pyridin-1(6H)-yl)pyrrolidin-1-yl propanenitrile (0.2 g, 0.4 mmol) was dissolved in a mixture of 5 mL tetrahydrofuran and 5 mL methanol, followed by adding 5 mL of 1 M sodium hydroxide, stir at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate=1:1) to yield LXS59: 3-((S)-3-(2-((R)-1-hydroxyethyl)imidazo[4,5-d]pyrrolo[2,3-b]pyridin-1(6H)-yl)pyrrolidin-1-yl)propanenitrile (0.1 g, 72%). ¹HNMR (300 MHz, DMSO-d₆): δ=11.99 (s, 1H), 8.83 (s, 1H), 7.59 (s, 1H), 6.88 (s, 1H), 4.51-4.69 (m, 1H), 3.65-3.85 (m, 1H), 3.05 (t, J=9.0 Hz, 2H), 2.76-2.89 (m, 2H), 2.73 (t, J=9.0 Hz, 2H), 2.20-2.41 (m, 2H), 1.95-2.22 (m, 2H), 1.43 (t, J=7.5 Hz, 3H) ppm; ¹³CNMR (75 MHz, DMSO-d₆) δ 148.61, 148.57, 142.11, 128.64, 126.11, 120.73, 118.57, 115.62, 99.38, 63.62, 58.93, 57.62, 55.17, 27.64, 22.89, 16.14 ppm; HRMS (ESI): m/z [M+H]⁺. C₁₇H₂₁N₆O calculated value 325.1771, measured value 325.1780.

### Example 60

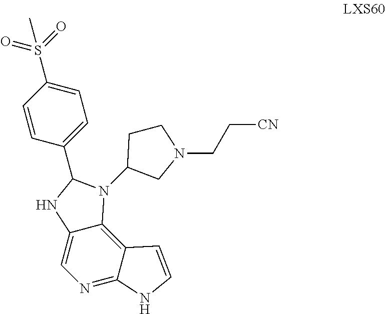 [SMILES: CS(=O)(=O)c1ccc(C2Nc3cnc4[nH]ccc4c3N2C2CCN(CCC#N)C2)cc1]

### 3-(3-(2-(4-(Methvlsulfonyl)phenyl)-2,3-dihydroimidazo[4,5-d]pyrrolo[2,3-b]pyridin-1(6H)-yl)pyrrolidin-1-yl)propanenitrile

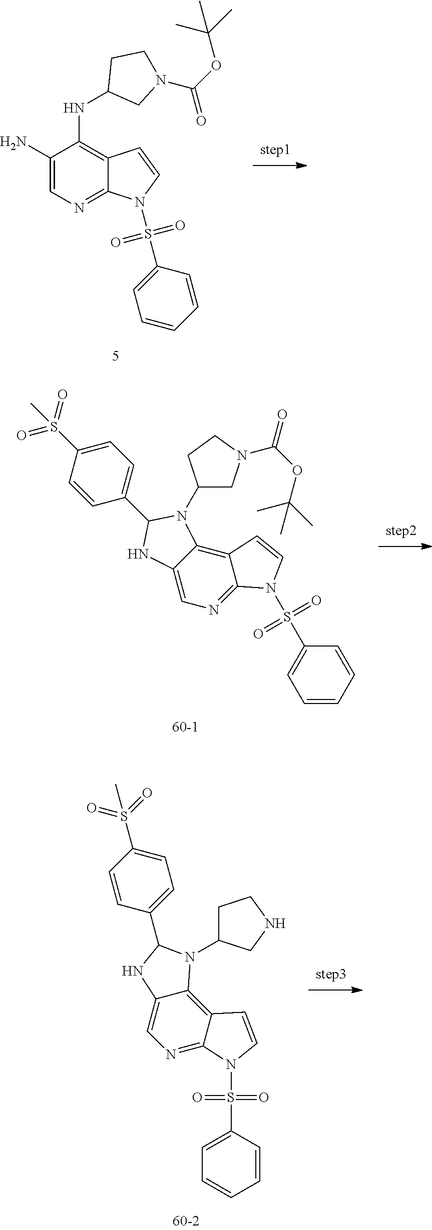 [SMILES: CC(C)(C)OC(=O)N1CCC(Nc2c(N)cnc3c2ccn3S(=O)(=O)c2ccccc2)C1 ; CC(C)(C)OC(=O)N1CCC(N2c3c(cnc4c3ccn4S(=O)(=O)c3ccccc3)NC2c2ccc(S(C)(=O)=O)cc2)C1 ; CS(=O)(=O)c1ccc(C2Nc3cnc4c(ccn4S(=O)(=O)c4ccccc4)c3N2C2CCNC2)cc1]

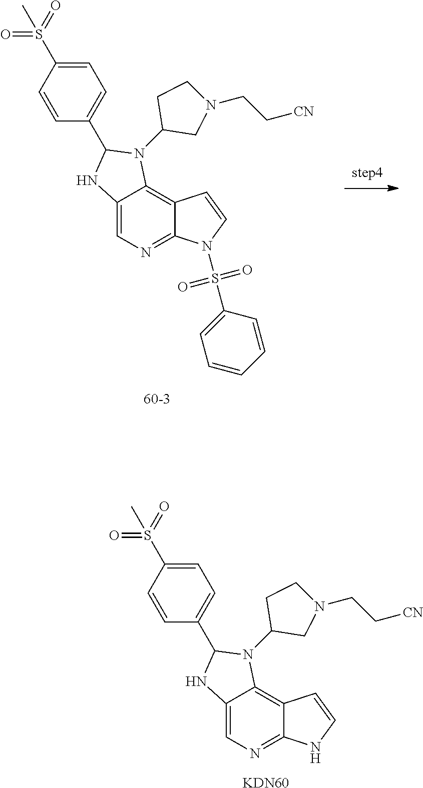

Step 1: Dissolve tert-butyl 3-(((5-amino-1-(phenylsulfonyl)-1H-pyrrolo[2,3-b]pyridin-4-yl)amino)pyrrolidine-1-carboxylate (0.3 g, 0.7 mmol) in 10 mL of DMF, add Na₂S₂O₅ (0.7 g, 3.5 mmol) followed by adding 4-methylsulfonylbenzaldehyde (0.2 g, 1.4 mmol). Stir for 12 h at 90° C. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum. The residue was purified using a silica gel column (petroleum ether:ethyl acetate=1:1) to yield 3-(2-(4-(methylsulfonyl)phenyl)-6-(phenylsulfonyl)-2,3-dihydroimidazo[4,5-d]pvrrolo[2,3-b]pyridin-1(6H)-pyrrolidine-1-carboxylate as a yellow solid (0.2 g, 49%). HRMS(ESI): m/z [M+H]⁺. CaoH34N₅O₆S₂ calculated value 624.1945, measured value 624.1955.

Step 2: Dissolve 3-(2-(4-(methylsulfonyl)phenyl)-6-(phenylsulfonyl)-2,3-dihydroimidazo[4,5-d]pyrrolo[2,3-b]pyridin-1(6H)-pyrrolidine-1-carboxylate (0.2 g, 0.3 mmol) in 10 mL of dichloromethane, slowly add trifluoroacetic acid (0.4 g, 3.0 mmol) and then stir for 12 h at room temperature. The product was then concentrated under vacuum to yield 2-(4-(methylsulfonyl)phenyl)-6-(phenylsulfonyl)-1-(pyrrolidin-3-yl)-1,2,3,6-tetrahydroimidazo[4,5-d]pyrrolo[2,3-b]pyridine as a light yellow oil (0.1 g, 60%). The product can be used directly in the next step without further purification. HRMS (ESI): m/z [M+H]⁺. C₂₅H₂₆N₅O₄S₂ calculated value 524.1421, measured value 524.1427.

Step 3: Dissolve 2-(4-(methylsulfonyl)phenyl)-6-(phenylsulfonyl)-1-(pyrrolidin-3-yl)-1,2,3,6-tetrahydroimidazo[4,5-d]pyrrolo[2,3-b]pyridine (0.1 g, 0.2 mmol) in 10 mL of tetrahydrofuran, add DIPEA (0.1 g, 0.4 mmol) followed by slow dropwise addition of 3-bromopropyl cyanide (0.1 g, 0.3 mmol). Stir at room temperature for 3 h. Monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield 3-(3-(2-(4-(methylsulfonyl)phenyl)-6-(phenylsulfonyl)-2,3-dihydroimidazo[4,5-d]pyrrolo[2,3-b]pyridin-1(6H)-yl)pyrrolidin-1-vl propanenitrile as a pale yellow oil (0.1 g, 91%). The product can be used directly in the next step without further purification. HRMS (ESI): m/z [M+H]⁺. C₂₈H₂₉N₆O₄S₂ calculated value 577.1686, measured value 577.1688.

Step 4: Dissolve 3-(3-(2-(4-(methylsulfonyl)phenyl)-6-(phenylsulfonyl)-2,3-dihydroimidazo[4,5-d]pyrrolo[2,3-b]pyridin-1(6H)-yl)pyrrolidin-1-yl propanenitrile (0.1 g, 0.2 mmol) in a mixture of 5 mL tetrahydrofuran and 5 mL methanol, add 5 mL of 1 M sodium hydroxide and then stir at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS60: 3-(3-(2-(4-(Methylsulfonyl)phenyl)-2,3-dihydroimidazo[4,5-d]pyrrolo[2,3-b]pyridin-1(6H)-yl)pyrrolidin-1-yl)propanenitrile (60.0 mg, 79%). ¹HNMR (300 MHz, DMSO-d₆): δ=11.46 (s, 1H), 7.89 (s, 1H), 7.74 (d, J=9.0 Hz, 2H), 7.56 (s, 1H), 7.51 (d, J=9.0 Hz, 2H), 6.88 (s, 1H), 5.04 (s, 1H), 3.32 (s, 3H), 3.03 (t, J=9.0 Hz, 2H), 2.75 (t, J=9.0 Hz, 2H), 2.66-2.71 (m, 1H), 2.31-2.56 (m, 2H), 2.19-2.33 (m, 2H), 1.66-2.02 (m, 2H) ppm; ¹³ CNMR (75 MHz, DMSO-d₆) δ 144.28, 139.28, 138.73, 134.49, 131.18, 128.95, 128.15, 127.15, 123.38, 119.04, 108.16, 99.38, 83.28, 62.18, 59.28, 55.41, 55.14, 47.72, 33.58, 17.10 ppm; HRMS (ESI): m/z [M+H]⁺. C₂₂H₂₅N₆O₂S calculated value 437.1754, measured value 437.1759.

### Example 61

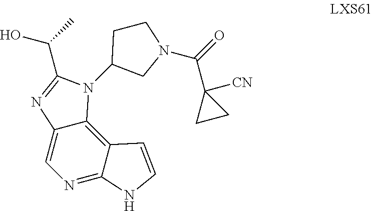 [SMILES: C[C@@H](O)c1nc2cnc3[nH]ccc3c2n1C1CCN(C(=O)C2(C#N)CC2)C1]

### 1-(3-(2-((R)—I-Hydroxyethyl)imidazo[4,5-d]pyrrolo[2,3-b]pyridin-1(6H)-yl)pyrrolidin-1-carbonyl)cyclopropanecarbonitrile

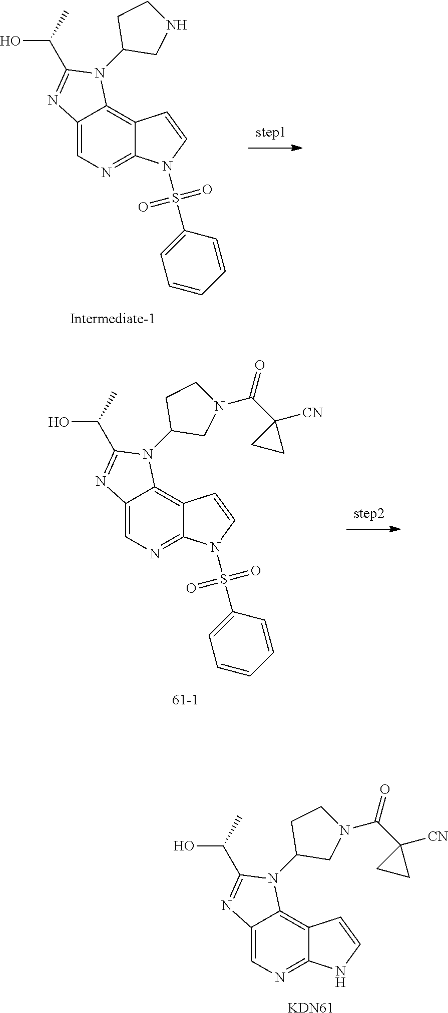

Step 1: Dissolve (1R)-1-(6-(phenylsulfonyl)-1-(pyrrolidin-3-yl)-1,6-dihydroimidazo[4,5-d]pyrrolo[2,3-b]pyridin-2-yl)ethanol (0.3 g, 0.7 mmol) in 10 mL of tetrahydrofuran, add DIPEA (0.2 g, 1.4 mmol) followed by slow dropwise addition of 3-bromopropacyanine (0.2 g, 1.1 mmol). Stir at room temperature for 3 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield 1-(3-(2-(R)-1-hydroxyethyl)-6-(phenylsulfonyl)imidazo[4,5-d]pyrrolo[2,3-b]pyridin-1(6H)-yl)pyrrolidin-1-carbonyl)cyclopropanecarbonitrile as a yellow oil (0.3 g, 82%). The product can be used directly in the next step without further purification. HRMS (ESI): m/z [M+H]⁺. C₂₅H₂₅N₆O₄S calculated value 505.1653, measured value 505.1660.

Step 2: 1-(3-(2-((R)-1-Hydroxyethyl)-6-(phenylsulfonyl)imidazo[4,5-d]pyrrolo[2,3-b]pyridin-1(6H)-yl)pyrrolidin-1-carbonyl)cyclopropanecarbonitrile (0.3 g, 0.6 mmol) was dissolved in a mixture of 5 mL tetrahydrofuran and 5 mL methanol, followed by the addition of 5 mL of 1 M sodium hydroxide. Stir at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum. The residue was purified using a silica gel column (petroleum ether:ethyl acetate=1:1) to yield LXS61: 1-(3-(2-((R)-1-Hydroxyethyl)imidazo[4,5-d]pyrrolo[2,3-b]pyridin-1(6H)-yl)pyrrolidin-1-carbonyl)cyclopropanecarbonitrile (0.1 g 46%). ¹HNMR (300 MHz, DMSO-d₆): δ=12.06 (s, 1H), 7.55 (s, 1H), 6.87 (s, 1H), 4.33-4.68 (m, 1H), 3.894.17 (m, 1H), 3.77-4.03 (m, 2H), 3.41-3.51 (m, 2H), 2.29-2.54 (m, 2H), 1.48 (d, J=9.0 Hz, 3H), 0.67-0.92 (m, 4H) ppm; ¹³CNMR (75 MHz, DMSO-d₆) δ 180.77, 148.66, 148.53, 142.17, 129.00, 127.17, 120.73, 115.63, 114.47, 99.39, 63.69, 58.42, 51.88, 46.48, 27.15, 22.84, 13.63, 10.06 ppm; HRMS (ESI): m/z [M+H]⁺. C₁₉H₂₁N₆O₂ calculated value 365.1721, measured value 365.1711.

### Example 62

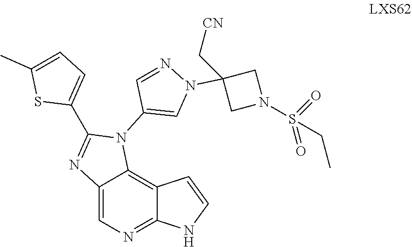 [SMILES: CCS(=O)(=O)N1CC(CC#N)(n2cc(-n3c(-c4ccc(C)s4)nc4cnc5[nH]ccc5c43)cn2)C1]

### 2-(1-(Ethylsulfonyl)-3-(4-(2-(5-methylthien-2-yl)imidazo[4,5-d]pyrrolo[2,3-b]pyridin-1(6H)-yl)-1H-pyrazol-1-yl)azetidin-3-yl)acetonitrile

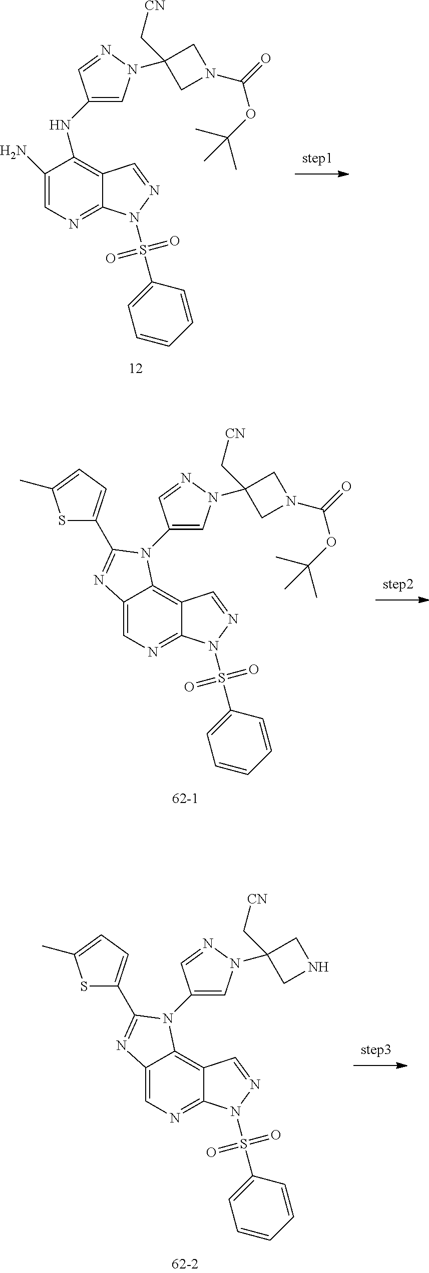 [SMILES: CC(C)(C)OC(=O)N1CC(CC#N)(n2cc(Nc3c(N)cnc4c3cnn4S(=O)(=O)c3ccccc3)cn2)C1 ; Cc1ccc(-c2nc3cnc4c(cnn4S(=O)(=O)c4ccccc4)c3n2-c2cnn(C3(CC#N)CN(C(=O)OC(C)(C)C)C3)c2)s1 ; Cc1ccc(-c2nc3cnc4c(cnn4S(=O)(=O)c4ccccc4)c3n2-c2cnn(C3(CC#N)CNC3)c2)s1]

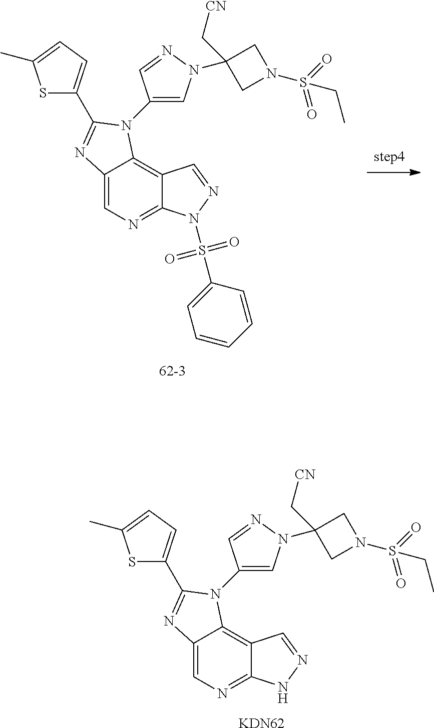

Step 1: Tert-butyl 3-(4-((5-amino-1-(phenylsulfonyl)-1H-pyrrolo[2,3-b]pyridin-4-yl)amino)-1H-pyrazol-1-yl)-3-(cyanomethyl)azetidine-1-carboxylate (0.3 g, 0.6 mmol) was dissolved in 10 mL of DMF, Na₂S₂O₅ (0.6 g, 3.0 mmol) was added, followed by the addition of 5-methyl-2-thiophenecarboxaldehyde (0.2 g, 0.9 mmol) dropwise and stirring for 12 h at 90° C. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum. The residue was purified using a silica gel column (petroleum ether:ethyl acetate=1:1) to yield tert-butyl 3-(cyanomethyl)-3-(4-(2-(5-methylthien-2-yl)-6-(phenylsulfonyl)imidazo[4,5-d]pyrrolo[2,3-b]pyridin-1(6H)-yl)-1H-pyrazol-1-yl) azetidine-1-carboxylate as a yellow solid (0.2 g, 56%). HRMS (ESI): m/z [M+H]⁺. C₃₂H₃₁N₅O₄S₂ calculated value 655.1904, measured value 655.1909.

Step 2: Tert-butyl 3-(cyanomethyl)-3-(4-(2-(5-methylthien-2-yl)-6-(phenylsulfonyl)imidazo[4,5-d]pyrrolo[2,3-b]pyridin-1(6H)-yl)-1H-pyrazol-1-yl)azetidine-1-carboxylate (0.2 g, 0.3 mmol) was dissolved in 10 mL of dichloromethane and then trifluoroacetic acid (0.4 g, 3.0 mmol) was slowly added and stirred at room temperature for 12 h. The product was concentrated under vacuum to yield 2-(3-(4-(2-(5-methylthien-2-yl)-6-(phenylsulfonyl)imidazo[4,5-d]pyrrolo[2,3-b]pyridin-1(6H)-yl)-1H-pyrazol-1-yl)azetidin-3-yl)acetonitrile as a light yellow oil (0.1 g, 59%). The product can be used directly in the next step without further purification. HRMS (ESI): m/z [M+H]⁺. C₂₇H₂₃N₅O₂S₂ calculated value 555.1380, measured value 555.1390.

Step 3: Dissolve 2-(3(4-(2-(5-methylthien-2-yl)-6-(phenylsulfonyl)imidazo[4,5-d]pyrrolo[2,3-b]pyridin-1(6H)-yl)-1H-pyrazol-1-yl)azetidin-3-yl)acetonitrile (0.1 g, 0.2 mmol) in 10 mL of tetrahydrofuran, add DIPEA (0.1 g, 0.4 mmol), followed by the slow dropwise addition of ethylsulfonyl chloride (0.1 g, 0.3 mmol), stir the mixture slowly for 3 h at room temperature. Monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield 2-(1-(ethylsulfonyl)-3-(4-(2-(5-methylthien-2-yl)-6-(phenylsulfonyl)imidazo[4,5-d]pyrrolo[2,3-b]pyridin-1(6H)-yl)-1H-pyrazol-1-yl)azetidin-3-yl)acetonitrile as a light yellow oil (0.1 g, 86%). The product can be used directly in the next step without further purification. HRMS(ESI): m/z [M+H]⁺. C₂₉H₂₇N₅O₄S₃ calculated value 647.1312, measured value 647.1318.

Step 4: 2-(1-(Ethylsulfonyl)-3-(4-(2-(5-methylthien-2-yl)-6-(phenylsulfonyl)imidazo[4,5-d]pyrrolo[2,3-b]pyridin-(6H)-yl)-1H-pyrazol-1-yl)azetidin-3-yl)acetonitrile (0.1 g, 0.2 mmol) was dissolved in a solvent mixture of 5 mL tetrahydrofuran and 5 mL methanol. Add 5 mL of 1 M sodium hydroxide and then stir at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate =1:1) to yield LXS62: 2-(1-(Ethylsulfonyl)-3-(4-(2-(5-methylthien-2-yl)imidazo[4,5-d]pyrrolo[2,3-b]pyridin-1(6H)-yl)-1H-pyrazol-1-yl)azetidin-3-yl)acetonitrile (65.0 mg, 83%). ¹ HNMR (300 MHz, DMSO-d₆): δ=12.03 (s, 1H), 8.89 (s, 1H), 8.08 (s, 2H), 7.65 (s, 1H), 7.49 (d, J=9.0 Hz, 1H), 6.89 (s, 1H), 6.84 (d, J=9.0 Hz, 1H), 3.89-4.06 (m, 4H), 3.45 (m, 2H), 2.81 (s, 2H), 2.36 (s, 3H), 1.22 (t, J=7.5 Hz, 3H) ppm; ¹³ C NMR (75 MHz, DMSO-d₆) δ 148.69, 142.17, 141.66, 141.61, 134.33, 130.77, 129.73, 129.01, 127.49, 127.11, 120.73, 117.74, 115.63, 100.54, 99.31, 59.29, 51.66, 50.83, 23.28, 15.22, 2.69 ppm. HRMS (ESI): m/[M+H]⁺. C₂₃H₂₃N₅O₂S₂ calculated value 507.1380, measured value 507.1390.

### Example 63

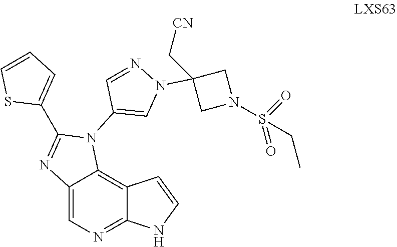 [SMILES: CCS(=O)(=O)N1CC(CC#N)(n2cc(-n3c(-c4cccs4)nc4cnc5[nH]ccc5c43)cn2)C1]

### 2-(1-(Ethylsulfonyl)-3-(4-(2-(thiophen-2-yl)imidazo[4,5-d]pyrrolo[2,3-b]pyridin-1(6H)-yl)-1H-pyrazol-1-yl)azetidin-3-yl)acetonitrile

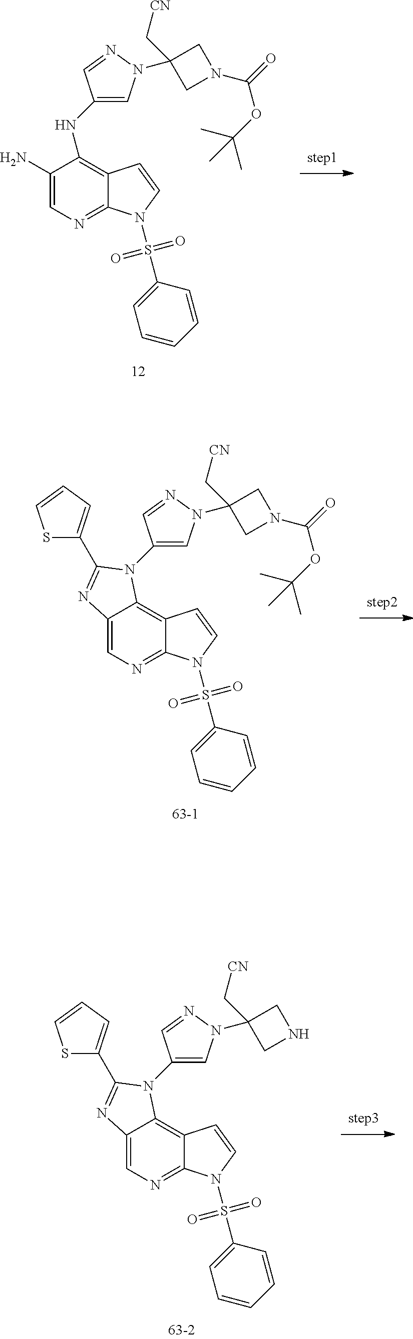 [SMILES: CC(C)(C)OC(=O)N1CC(CC#N)(n2cc(Nc3c(N)cnc4c3ccn4S(=O)(=O)c3ccccc3)cn2)C1 ; CC(C)(C)OC(=O)N1CC(CC#N)(n2cc(-n3c(-c4cccs4)nc4cnc5c(ccn5S(=O)(=O)c5ccccc5)c43)cn2)C1 ; N#CCC1(n2cc(-n3c(-c4cccs4)nc4cnc5c(ccn5S(=O)(=O)c5ccccc5)c43)cn2)CNC1]

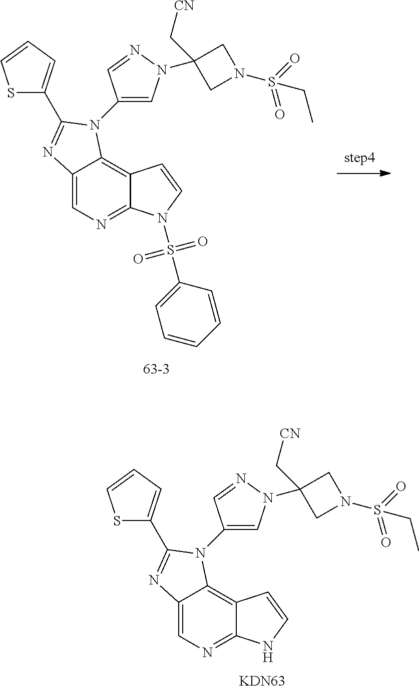

Step 1: Dissolve tert-butyl 3-(4-((5-amino-1-(phenylsulfonyl)-1H-pyrrolo[2,3-b]pyridin-4-yl)amino)-1H-pyrazol-1-yl)-3-(cyanomethyl)azetidine-1-carboxylate (0.3 g, 0.6 mmol) in 10 mL of DMF, add Na₂S₂O₅ (0.6 g, 3.0 mmol). After adding 2-thiophenecarboxaldehyde (0.2 g, 0.9 mmol) dropwise, raise the temperature to 90° C. and stir for 12 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum. The residue was purified using a silica gel column (petroleum ether:ethyl acetate=1:1) to yield tert-butyl 3-(cyanomethyl)-3-(4-(6-(phenylsulfonyl)-2-(thiophen-2-yl)imidazo[4,5-d]pyrrolo[2,3-b]pyridin-1(6H)-yl)-1H-pyrazol-1-yl)azetidine-1-carboxylate as a yellow solid (0.2 g, 57%). HRMS (ESI): m/z [M+H]⁺. C₃₁H₂₉N₅O₄S₂ calculated value 641.1748, measured value 641.1751.

Step 2: Tert-butyl 3-(cyanomethyl)-3-(4-(6-(phenylsulfonyl)-2-(thiophen-2-yl)imidazo[4,5-d]pyrrolo[2,3-b]pyridin-1(6H)-yl)-1H-pyrazol-1-yl)azetidine-1-carboxylate (0.2 g, 0.3 mmol) was dissolved in 10 mL ofdichloromethane, and then trifluoroacetic acid (0.4 g, 3.0 mmol) was slowly added and stirred at room temperature for 12 hours. The product was concentrated under vacuum to yield 2-(3-(4-(6-(phenylsulfonyl)-2-(thiophen-2-yl)imidazo[4,5-d]pyrrolo[2,3-b]pyridin-1(6H)-yl)-1H-pyrazol-1-yl)azetidin-3-yl)acetonitrile as a light yellow oil (0.1 g, 59%). The product can be used directly in the next step without further purification. HRMS (ESI): m/z [M+H]⁺. C₂₆H₂₁N₅O₂S₂ calculated value 541.1223, measured value 541.1230.

Step 3: Dissolve 2-(3-(4-(6-(phenylsulfonyl)-2-(thiophen-2-yl)imidazo[4,5-d]pyrrolo[2,3-b]pyridin-1(6H)-yl)-1H-pyrazol-1-yl)azetidin-3-yl)acetonitrile (0.1 g, 0.2 mmol) in 10 mL of tetrahydrofuran, add DIPEA (0.1 g, 0.4 mmol), followed by the dropwise addition of ethylsulfonyl chloride (0.1 g, 0.3 mmol), and then stir slowly for 3 h at room temperature. Monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield 2-(3-(4-(6-(phenylsulfonyl)-2-(thiophen-2-yl)imidazo[4,5-d]pyrrolo[2,3-]pyridin-1(6H)-yl)-1H-pyrazol-1-yl)azetidin-3-yl)acetonitrile as a pale yellow oil (0.1 g, 85%). The product can be used directly in the next step without further purification. HRMS (ESI): m/z [M+H]⁺. C₂₈H₂₅N₅O₄S₃ calculated value 633.1155, measured value 633.1160.

Step 4: 2-(3-(4-(6-(Phenylsulfonyl)-2-(thiophen-2-yl)imidazo[4,5-d]pyrrolo[2,3-b]pyridin-1(6H)-yl)-1H-pyrazol-1-yl)azetidin-3-yl)acetonitrile (0.1 g, 0.2 mmol) was dissolved in a solvent mixture of 5 mL tetrahydrofuran and 5 mL methanol, and 5 mL of 1M sodium hydroxide was added and then stirred at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate=1:1) to yield LXS63: 2-(1-(Ethylsulfonyl)-3-(4-(2-(thiophen-2-yl)imidazo[4,5-d]pyrrolo[2,3-b]pyridin-1(6H)-yl)-1H-pyrazol-1-yl)azetidin-3-yl)acetonitrile (60.0 mg, 77%). ¹HNMR (300 MHz, DMSO-d₆): δ=11.96 (s, 1H), 8.74 (s, 1H), 8.10 (s, 2H), 7.85 (d, J=7.5 Hz, 1H), 7.69 (d, J=7.5 Hz, 1H), 7.67 (s, 1H), 7.13-7.21 (m, 1H), 6.79 (s, 1H), 3.88-4.16 (m, 4H), 3.55 (m, 2H), 2.86 (s, 2H), 1.22 (t, J=7.5 Hz, 3H) ppm; ¹³CNMR (75 MHz, DMSO-d₆) δ 148.67, 143.88, 142.19, 141.53, 130.77, 129.73, 129.04, 128.63, 128.04, 127.17, 120.73, 117.72, 115.69, 100.53, 99.38, 59.28, 51.64, 50.88, 23.37, 5.77 ppm: HRMS (ESI): m/z [M+H]⁺. C₂₂H₂₁N₅O₂S₂ calculated value 493.1223, measured value 493.1230.

### Example 64

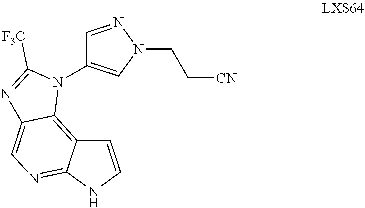 [SMILES: N#CCCn1cc(-n2c(C(F)(F)F)nc3cnc4[nH]ccc4c32)cn1]

### 3-(4-(2-(Trifluoromethyl)imidazo[4,5-d]pyrrolo[2,3-b]pyridin-1(6H)-yl)-1H-pyrazol-1-yl)propanenitrile

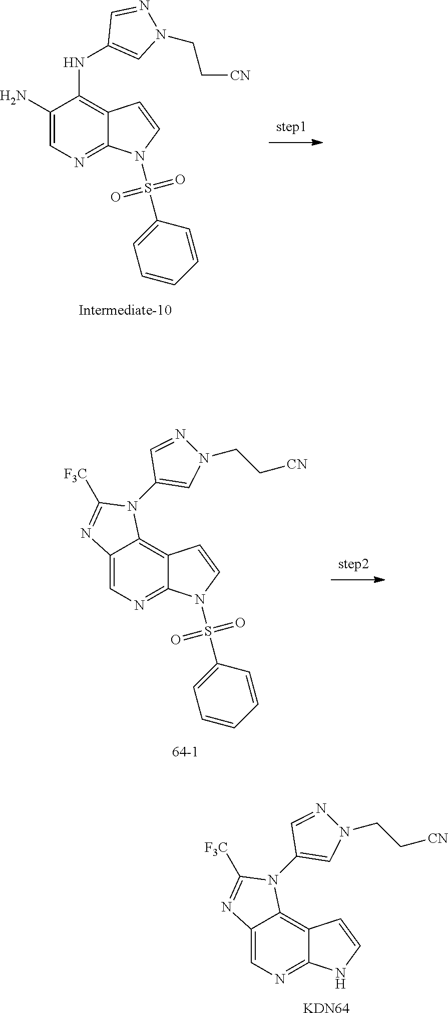

Step 1: Triethyloxonium tetrafluoroborate (0.4 g, 2.1 mmol) and 2,2,2-trifluoroacetamide (0.2 g, 2.1 mmol) were dissolved in 10 mL of tetrahydrofuran, stirred at room temperature for 3 h and concentrated under vacuum to obtain the mixture in the form of oil, followed by the addition of 10 mL of ethanol to dissolve and the addition of 3-(4-((5-amino-1-(phenylsulfonyl)-1H-pyrrolo[2,3-b]pyridin-4-yl)amino-1H-pyrazol-1-yl)propanenitrile (0.3 g, 0.7 mmol). Raise the temperature to maintain reflux and stir for 3 h. The reaction was monitored by TLC for completion. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield 3-(4-(6-(phenylsulfonyl)-2-(trifluoromethyl)imidazo[4,5-d]pyrrolo[2,3-b]pyridin-1(6H)-yl)-1H-pyrazol-1-yl)propanenitrile as a milk white oil (0.3 g, 84%). HRMS (ESI): m/z [M+H]⁺. C₂₁H₁₅F₃N₇O₂S calculated value 486.0955, measured value 486.0961.

Step 2: Dissolve 3-(4-(6-(phenylsulfonyl)-2-(trifluoromethyl)imidazo[4,5-d]pyrrolo[2,3-b]pyridin-1(6H)-yl)-1H-pyrazol-1-yl) propanenitrile (0.3 g, 0.6 mmol) in a mixture of 5 mL of tetrahydrofuran and 5 mL of methanol, and stir for 5 h at room temperature after adding 5 mL of 1 M sodium hydroxide. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS64: 3-(4-(2-(Trifluoromethyl)imidazo[4,5-d]pyrrolo[2,3-b]pyridin-1(6H)-yl)-1H-pyrazol-1-yl)propanenitrile (0.1 g, 47%). ¹HNMR (300 MHz, DMSO-d₆): δ=11.96 (s, 1H), 8.89 (s, 1H), 8.06 (s, 1H), 7.94 (s, 1H), 7.56 (s, 1H), 6.86 (s, 1H), 5.04 (t, J=9.0 Hz, 2H), 3.24 (t, J=9.0 Hz, 2H) ppm; ¹³CNMR (75 MHz, DMSO-d₆) δ 148.69, 144.57, 142.18, 130.77, 129.84, 129.04, 127.11, 120.73, 117.77, 116.73, 115.69, 100.52, 99.67, 49.28, 16.88 ppm. HRMS (ESI): m/z [M+H]⁺. C₁₅H₁₁F₃N₇ calculated value 346.1023, measured value 346.1029.

### Example 65

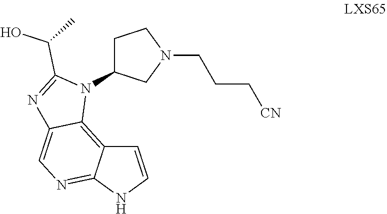 [SMILES: C[C@@H](O)c1nc2cnc3[nH]ccc3c2n1[C@H]1CCN(CCCC#N)C1]

### 4-((S)-3-(2-((R)-1-Hydroxyethyl)imidazo[4,5-d]pyrrolo[2,3-b]pyridin-1(6H)-yl)pyrrolidin-1-yl)butanenitrile

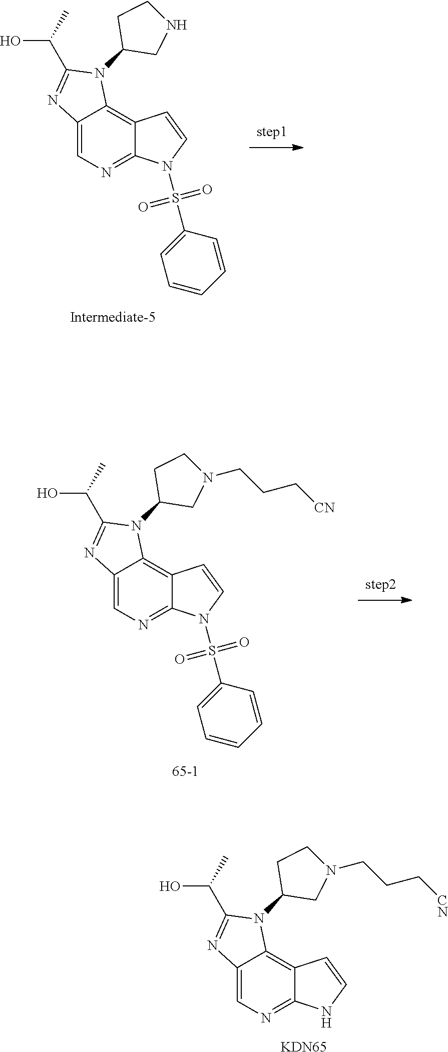

Step 1: Dissolve (R)-1-(6-(phenylsulfonyl)-1-((S-pyrrolidin-3-yl)-1,6-dihydroimidazo[4,5-d]pyrrolo[2,3-b]pyridin-2-yl)ethanol (0.3 g, 0.7 mmol) in 10 mL of tetrahydrofuran, add DIPEA (0.2 g, 1.4 mmol) and then slowly add 4-bromobutyronitrile (0.2 g, 1.1 mmol) dropwise. Raise the temperature to maintain reflux and stir for 3 h. Monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield 4-((S)-3-(2-((R)-1-hydroxyethyl)-6-(phenylsulfonyl)imidazo[4,5-d]pyrrolo[2,3-b]pyridin-1(6H)-yl)pyrrolidin-1-yl)butyronitrile as a pale yellow oil (0.2 g, 57%). The product can be used directly in the next step without further purification. HRMS(ESI): m/z [M+H]⁺. C₂₄H₂₇N₆O₃S calculated value 479.1860, measured value 479.1871.

Step 2: 4-((S)-3-(2-((R)-1-Hydroxyethyl)-6-(phenylsulfonyl)imidazo[4,5-d]pyrrolo[2,3-b]pyridin-1(6H)-yl)pyrrolidin-1-yl)butyronitrile (0.2 g, 0.4 mmol) was dissolved in a mixture of 5 mL of tetrahydrofuran and 5 mL of methanol, followed by the addition of 5 mL of IM sodium hydroxide. Stir at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate=1:1) to yield LXS65: 4-((S)-3-(2-((R)-1-Hydroxyethyl)imidazo[4,5-d]pyrrolo[2,3-b]pyridin-1(6H)-yl)pyrrolidin-1-yl)butanenitrile (0.1 g, 71%). ¹HNMR (300 MHz, DMSO-d₆): δ=12.16 (s, 1H), 8.89 (s, 1H), 7.69 (s, 1H), 6.88 (s, 1H), 4.53-4.69 (m, 11H), 3.69-3.80 (m, 1H), 2.56-2.81 (m, 2H), 2.43 (t, J=7.5 Hz, 2H), 2.21-2.31 (m, 2H), 1.92-2.16 (m, 2H), 1.87 (t, J=7.5 Hz, 2H), 1.66-1.78 (m, 2H), 1.44 (d, J=9.0 Hz, 3H) ppm; ¹³CNMR (75 MHz, DMSO-d₆) δ 148.65, 148.51, 142.17, 129.04, 127.19, 120.77, 119.37, 115.62, 99.74, 63.67, 58.92, 58.49, 55.91, 55.53, 27.63, 22.81, 15.92, 15.19 ppm; HRMS (ESI): m/z [M+H]⁺. C₁₈H₂₃N₆O calculated value 339.1928, measured value 339.1931.

### Example 66

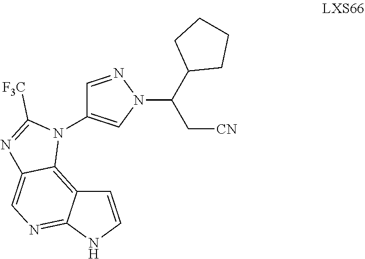 [SMILES: N#CCC(C1CCCC1)n1cc(-n2c(C(F)(F)F)nc3cnc4[nH]ccc4c32)cn1]

### 3-Cyclopentyl-3-(4-(2-(trifluoromethyl)imidazo[4,5-d]pyrrolo[2,3-b]pyridin-1(6H)-yl)-1H-pyrazol-1-yl)propanenitrile

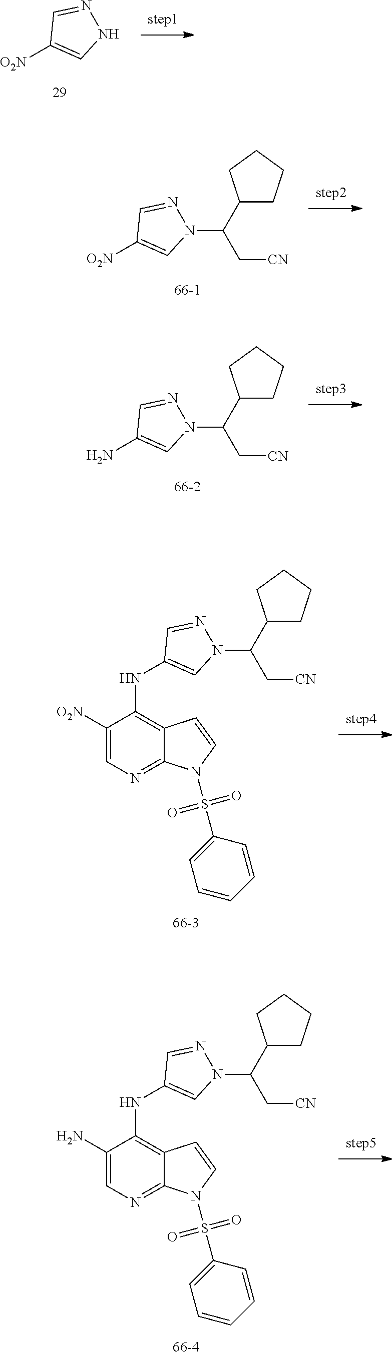 [SMILES: O=[N+]([O-])c1cn[nH]c1 ; N#CCC(C1CCCC1)n1cc([N+](=O)[O-])cn1 ; N#CCC(C1CCCC1)n1cc(N)cn1 ; N#CCC(C1CCCC1)n1cc(Nc2c([N+](=O)[O-])cnc3c2ccn3S(=O)(=O)c2ccccc2)cn1 ; N#CCC(C1CCCC1)n1cc(Nc2c(N)cnc3c2ccn3S(=O)(=O)c2ccccc2)cn1]

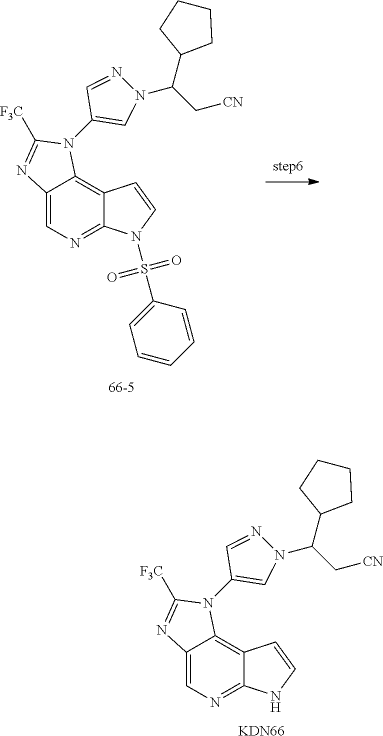

Step 1: Dissolve 4-nitropyrazole (0.5 g, 4.4 mmol) in 10 mL of tetrahydrofuran, add DIPEA (1.2 g, 8.8 mmol) followed by slow dropwise addition of 3-bromo-3-cyclopentylpropanenitrile (1.3 g, 6.6 mmol). Raise the temperature to maintain reflux and stir for 3 h. Monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield 3-cyclopentyl-3-(4-nitro-1H-pyrazol-1-yl)propanenitrile as a pale yellow oil (0.8 g, 77%). The product can be used directly in the next step without further purification. HRMS(ESI): m/z [M+H]⁺. C₁₁H₁₅N₄O₂ calculated value 235.1190, measured value 235.1194.

Step 2: 3-Cyclopentyl-3-(4-nitro-1H-pyrazol-1-yl)propanenitrile (0.8 g, 3.4 mmol) was dissolved in 10 mL of methanol, after adding palladium carbon (0.1 g, 10%), use hydrogen to replace the air in the reaction flask more than three times and the reaction was kept in hydrogen atmosphere. Stir at room temperature for 12 h. The reaction was monitored for completion by TLC. After filtration, the filtrate was collected and concentrated under vacuum to obtain 3-cyclopentyl-3-(4-amino-1H-pyrazol-1-yl)propanenitrile as a pink foamy solid (0.7 g, 100%). The product can be used directly in the next step without further purification. HRMS (ESI): m/z [M+H]⁺. C₁₁H₁₇N₄ calculated value 205.1448, measured value 205.1552.

Step 3: Dissolve 3-cyclopentyl-3-(4-amino-1H-pyrazol-1-yl)propanenitrile (0.7 g, 3.4 mmol) in 10 mL of tetrahydrofuran, add DIPEA (0.9 g, 6.8 mmol) followed by adding 4-chloro-5-nitro-1-(phenylsulfonyl)-1H-pyrrolo[2,3-b]pyridine (0.8 g, 2.3 mmol). Raise the temperature to maintain reflux and stir for 3 h. The reaction was monitored for completion by TLC. Saturated sodium bicarbonate was added until the reaction solution was weakly basic, and the organic phase was separated. The aqueous phase was extracted twice with dichloromethane. The organic phases were combined and washed with saturated saline, dried over anhydrous sodium sulfate and concentrated under vacuum. Triturate and cure with an appropriate amount of methanol to yield 3-cyclopentyl-3-(4-((5-nitro-1-(phenylsulfonyl)-1H-pyrrolo[2,3-b]pyridin-4-yl)amino)-1H-pyrazol-1-yl)propanenitrile as a pale yellow oil (0.6 g, 50%). The product can be used directly in the next step without further purification. HRMS (ESI): m/z [M+H]⁺. C₂₄H₂₄N₇O₄S calculated value 506.1605, measured value 506.1613.

Step 4: 3-Cyclopentyl-3-(4-((5-nitro-1-(phenylsulfonyl)-1H-pyrrolo[2,3-b]pyridin-4-yl)amino)-1H-pyrazol-1-yl)propanenitrile (0.6 g, 1.2 mmol) was dissolved in 10 mL of methanol, palladium carbon (0.1 g, 10%) was added and the air in the reaction flask was replaced more than three times using hydrogen. The reaction was carried out in hydrogen atmosphere and stirred at room temperature for 12 h. The reaction was monitored for completion by TLC. After filtration, the filtrate was collected and concentrated under vacuum to yield 3-cyclopentyl-3-(4-((5-amino-1-(phenylsulfonyl)-1H-pyrrolo[2,3-b]pyridin-4-yl)amino)-1H-pyrazol-1-yl)propanenitrile as a pink foamy solid (0.5 g, 88%). The product can be used directly in the next step without further purification. HRMS (ESI): m/z [M+H]⁺. C₂₄H₂₆N₇O₂S calculated value 476.1863, measured value 476.1870.

Step 5: Triethyloxonium tetrafluoroborate (0.6 g, 3.3 mmol) and 2,2,2-trifluoroacetamide (0.1 g, 3.3 mmol) were dissolved in 10 mL of tetrahydrofuran, stirred at room temperature for 3 h and concentrated under vacuum to obtain the mixture as an oil, followed by the addition of 10 mL of ethanol to dissolve and the addition of 3-cyclopentyl-3-(4-((5-amino-1-(phenylsulfonyl)-1H-pyrrolo[2,3-b]pyridin-4-yl)amino)-1H-pyrazol-1-yl)propanenitrile (0.5 g, 1.1 mmol). Raise the temperature to maintain reflux and stir for 3 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum. The residue was purified using a silica gel column (petroleum ether:ethyl acetate=2:1) to yield 3-cyclopentyl-3-(4-(6-(phenylsulfonyl)-2-(trifluoromethyl)imidazo[4,5-d]pyrrolo[2,3-b]pyridin-1(6H)-yl)-1H-pyrazol-1-yl) propanenitrile as a light green oil (0.2 g, 34%). HRMS(ESI): m/z [M+H]⁺. C₂₆H₂₃F₃N₇O₂S calculated value 554.1581, measured value 554.1593.

Step 6: 3-Cyclopentyl-3-(4-(6-(phenylsulfonyl)-2-(trifluoromethyl)imidazo[4,5-d]pyrrolo[2,3-b]pyridin-1(6H)-yl)-1H-pyrazol-1-yl)propanenitrile (0.2 g, 0.4 mmol) was dissolved in a solvent mixture of 5 mL tetrahydrofuran and 5 mL methanol, followed by the addition of 5 mL of 1 M sodium hydroxide. Stir for 5 h at room temperature and monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate=1:1) to yield LXS66: 3-Cyclopentyl-3-(4-(2-(trifluoromethyl)imidazo[4,5-d]pyrrolo[2,3-b]pyridin-1(6H)-yl)-1H-pyrazol-1-yl)propanenitrile (0.1 g, 67%). ¹HNMR (300 MHz, DMSO-d₆): δ=11.96 (s, 1H), 8.79 (s, 1H), 8.06 (s, 1H), 7.94 (s, 1H), 7.58 (s, 1H), 6.77 (s, 1H), 3.53-3.70 (m, 1H), 2.80 (d, J=9.0 Hz, 2H), 1.35-1.60 (m, 9H) ppm; ¹³CNMR (75 MHz, DMSO-d₆) δ 148.66, 144.67, 142.19, 130.78, 129.77, 129.03, 127.16, 120.74, 117.74, 116.74, 115.64, 100.58, 99.39, 63.95, 34.28, 30.95, 25.16, 18.88 ppm; HRMS (ESI): m/z [M+H]⁺. C₂₀H₁₉F₃N₇ calculated value 414.1649, measured value 414.1654.

### Example 67

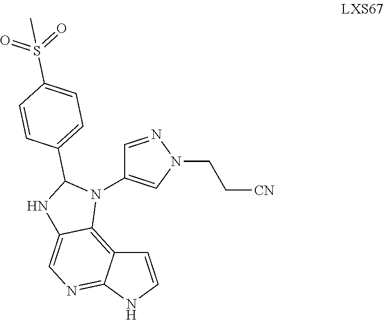 [SMILES: CS(=O)(=O)c1ccc(C2Nc3cnc4[nH]ccc4c3N2c2cnn(CCC#N)c2)cc1]

### 3-(4-(2-(4-(Methylsulfonyl)phenyl)-2,3-dihydroimidazo[4,5-d]pyrrolo[2,3-b]pyridin-1(6H)-yl)-1H-pyrazol-1-yl)propanenitrile

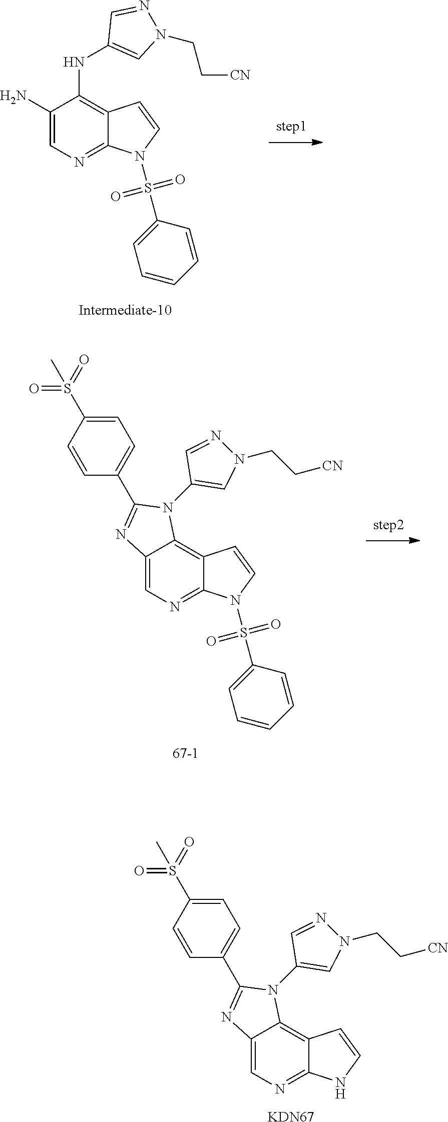

Step 1: Dissolve 3-(4-((5-amino-1-(phenylsulfonyl)-1H-pyrrolo[2,3-b]pyridin-4-yl)amino)-1H-pyrazol-1-yl)propanenitrile (0.3 g, 0.7 mmol) in 10 mL of DMF, add Na₂S₂O₅ (0.7 g, 3.5 mmol) followed by adding 4-methylsulfonylbenzaldehyde (0.2 g, 0.9 mmol) and stir for 12 h at 90° C. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline and dried over anhydrous sodium sulfate to yield 3-(4-(2-(4-(methylsulfonyl)phenyl)-6-(phenylsulfonyl)-2,3-dihydroimidazo[4,5-d]pyrrolo[2,3-b]pyridin-1(6H)-yl)-1H-pyrazol-1-yl)propanenitrile as a yellow solid (0.2 g, 47%). HRMS (ESI): m/z [M+H]⁺. C₂₇H₂₄N₇O₄S₂S calculated value 574.1326, measured value 574.1331.

Step 2: 3-(4-(2-(4-(Methylsulfonyl)phenyl)-6-(phenylsulfonyl)-2,3-dihydroimidazo[4,5-d]pyrrolo[2,3-b]pyridin-1(6H)-yl)-1H-pyrazol-1-yl)propanenitrile (0.2 g, 0.3 mmol) was dissolved in a solvent mixture of 5 mL tetrahydrofuran and 5 mL methanol followed by adding 1 M sodium hydroxide 5 mL and then stirred at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate =1:1) to yield LXS67: 3-(4-(2-(4-(methylsulfonyl)phenyl)-2,3-dihydroimidazo[4,5-d]pyrrolo[2,3-b]pyridin-1(6H)-yl)-1H-pyrazol-1-yl)propanenitrile (0.1 g, 66%). ¹ HNMR (300 MHz, DMSO-d₆): δ=11.06 (s, 11H), 7.89 (s, 1H), 7.86 (s, 1H), 7.74 (d, J=9.0 Hz, 2H), 7.58 (s, 1H), 7.51 (d, J=9.0 Hz, 2H), 7.18 (s, 1H), 6.87 (s, 1H), 5.04 (t, J=7.5 Hz, 3H), 3.34 (s, 3H), 3.20 (t, J=7.5 Hz, 2H) ppm; 13CNMR (75 MHz, DMSO-d₆) δ 149.78, 139.07, 138.77, 134.44, 131.17, 130.48, 130.15, 128.19, 127.94, 127.18, 123.34, 117.79, 115.75, 108.15, 99.43, 87.59, 49.22, 47.78, 17.88 ppm; HRMS (ESI): m/z [M+H]⁺. C₂₁H₂₀N₇O₂S calculated value 434.1394, measured value 434.1400.

### Example 68

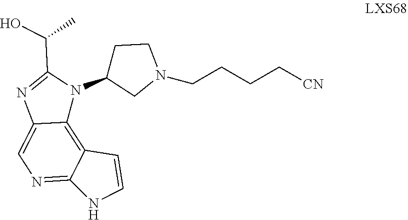 [SMILES: C[C@@H](O)c1nc2cnc3[nH]ccc3c2n1[C@H]1CCN(CCCCC#N)C1]

### 5-((S)-3-(2-((R)-1-Hydroxyethyl)imidazo[4,5-d]pyrrolo[2,3-b]pyridin-1(6H)-yl)pyrrolidin-1-yl)pentanonitrile

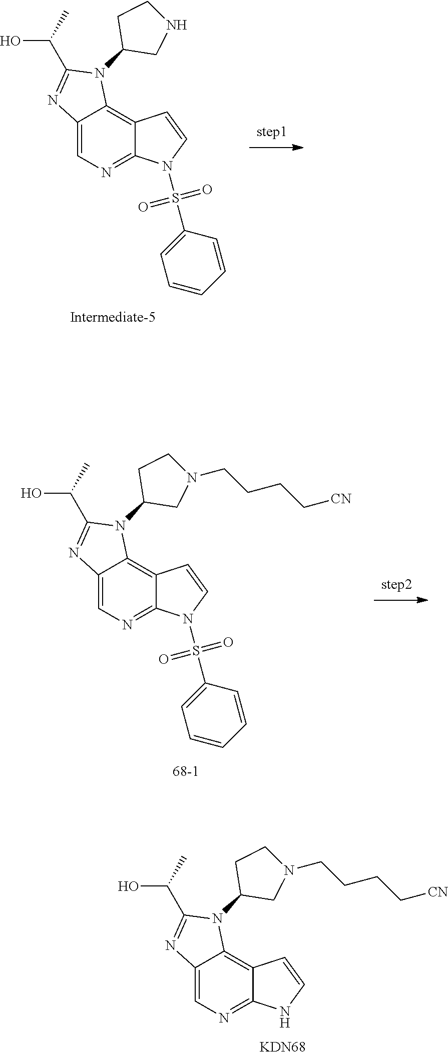

Step 1: Dissolve (R)-1-(6-(phenylsulfonyl)-1-((S-pyrrolidin-3-yl)-1,6-dihydroimidazo[4,5-d]pyrrolo[2,3-b]pyridin-2-yl)ethanol (0.3 g, 0.7 mmol) in 10 mL of tetrahydrofuran, add DIPEA (0.2 g, 1.4 mmol) and then slowly add 5-bromopentanonitrile (0.2 g, 1.1 mmol) dropwise. Raise the temperature to maintain reflux and stir for 3 h. Monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield 5-((S)-3-(2-((R)-1-hydroxyethyl)-6-(phenylsulfonyl)imidazo[4,5-d]pyrrolo[2,3-b]pyridin-1(6H)-yl)pyrrolidin-1-yl) pentanonitrile as a pale yellow oil (0.2 g, 56%). The product can be used directly in the next step without further purification. HRMS (ESI): m/z [M+H]⁺. C₂₅H₂₉N₆O₃S calculated value 493.2016, measured value 493.2020.

Step 2: Dissolve 5-((S)-3-(2-((R)-1-hydroxyethyl)-6-(phenylsulfonyl)imidazo[4,5-d]pyrrolo[2,3-b]pyridin-1(6H)-yl)pyrrolidin-1-yl) pentanonitrile (0.2 g, 0.4 mmol) in a mixture of 5 mL tetrahydrofuran and 5 mL methanol, followed by adding 5 mL of 1 M sodium hydroxide and stirring at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate=1:1) to yield LXS68: 5-((S)-3-(2-((R)-1-Hydroxyethyl)imidazo[4,5-d]pyrrolo[2,3-b]pyridin-1(6H)-yl)pyrrolidin-1-yl)pentanonitrile (0.1 g, 70%). ¹HNMR (300 MHz, DMSO-d₆): δ=12.03 (s, 1H), 8.87 (s, 1H), 7.58 (s, 1H), 6.85 (s, 1H), 4.594.68 (m, 1H), 3.65-3.79 (m, 1H), 2.58-2.83 (m, 2H), 2,44 (t, J=9.0 Hz, 2H), 2.19-2.28 (m, 2H), 1.90-2.13 (m, 2H), 1.90 (t, J=9.0 Hz, 2H), 1.63-1.77 (m, 2H), 1.48 (d, J=9.0 Hz, 3H), 1.25-1.30 (m, 2H) ppm; ¹³CNMR (75 MHz, DMSO-d₆) δ 148.94, 148.51, 142.11, 129.75, 127.63, 120.71, 119.39, 115.67, 99.52, 62.68, 58.93, 58.41, 55.96, 55.81, 27.63, 27.21, 23.22, 22.84, 17.18 ppm; HRMS (ESI): m/z [M+H]⁺. C₁₉H₂₅N₆O calculated value 353.2084, measured value 353.2088.

### Example 69

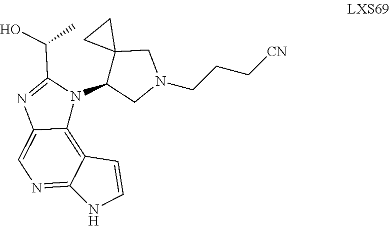 [SMILES: C[C@@H](O)c1nc2cnc3[nH]ccc3c2n1[C@@H]1CN(CCCC#N)CC12CC2]

### 4-((S)-7-(2-((R)-1-Hydroxyethyl)imidazo[4,5-d]pyrrolo[2,3-b]pyridin-1(6H)-yl)-5-azaspiro[2,4]hept-5-yl)butanenitrile

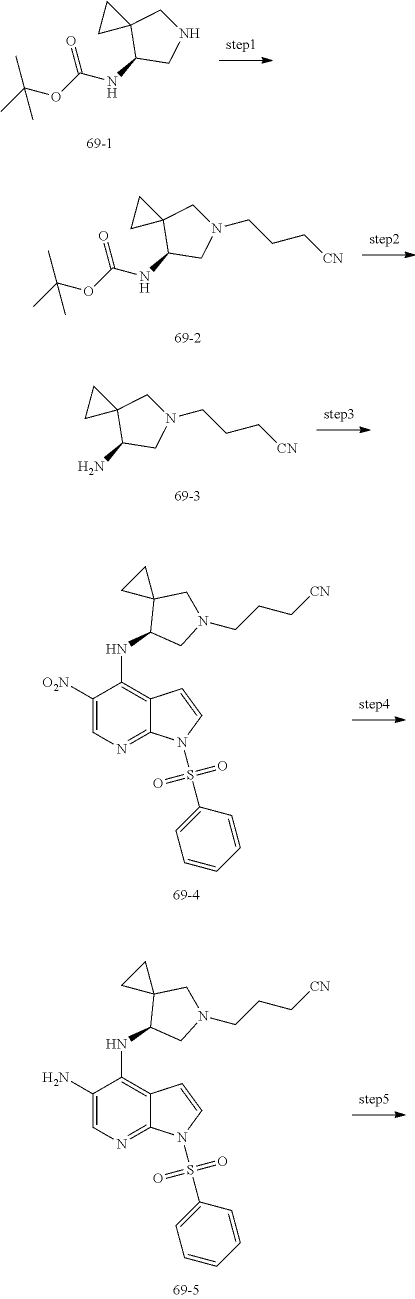 [SMILES: CC(C)(C)OC(=O)N[C@@H]1CNCC12CC2 ; CC(C)(C)OC(=O)N[C@@H]1CN(CCCC#N)CC12CC2 ; N#CCCCN1C[C@@H](N)C2(CC2)C1 ; N#CCCCN1C[C@@H](Nc2c([N+](=O)[O-])cnc3c2ccn3S(=O)(=O)c2ccccc2)C2(CC2)C1 ; N#CCCCN1C[C@@H](Nc2c(N)cnc3c2ccn3S(=O)(=O)c2ccccc2)C2(CC2)C1]

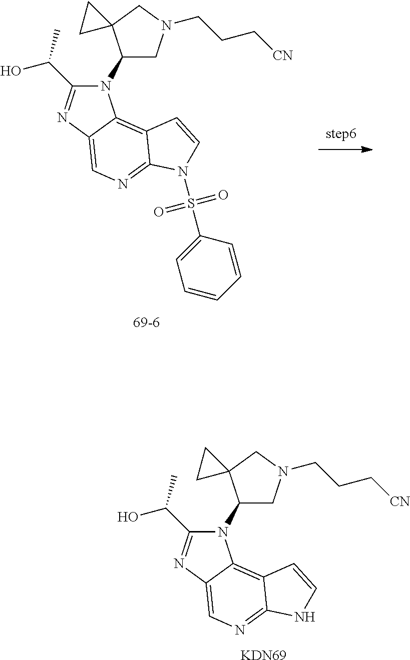

Step 1: Disslovetert-butyl (S)₅-azaspiro[2,4]hept-7-carbamate (0.3 g, 1.4 mmol) in 10 mL of DMF, add potassium carbonate (0.4 g, 2.8 mmol) and 4-broinobutanenitrile (0.3 g, 2.1 mmol) and stir at room temperature for 12 h. Monitor the reaction for completion by MS. Saturated sodium bicarbonate was added until the reaction solution was weakly basic, and the organic phase was separated. The aqueous phase was extracted three times using dichloromethane. The organic phases were combined and washed with saturated saline, dried over anhydrous sodium sulfate and concentrated under vacuum to yield (S)-tert-butyl (5-(3-cyanopropyl)-5-azaspiro[2,4]hept-7-yl)carbamate as a pale yellow oil (0.3 g, 76%) The product can be used directly in the next step without further purification. HRMS (ESI): nm/z [M+H]⁺. C₁₈H₂₆N₃O₂ calculated value 280.2020, measured value 280.2029.

Step 2: Dissolve (S)-tert-butyl (5-(3-cyanopropyl)-5-azaspiro[2,4]hept-7-yl)carbamate (0.3 g, 1.1 mmol) in 10 mL dichloromethane, slowly add trifluoroacetic acid (1.3 g, 11.0 mmol) and then stir at room temperature for 12 h. Concentrate under vacuum to obtain (S)-4-(7-amino-5-azaspiro[2,4]hept-5-yl)butanenitrile as a light yellow oil (0.2 g, 100%). The product can be used directly in the next step without further purification. HRMS (ESI): m/z [M+H]⁺. C₁₀H₁₈N₃ calculated value 180.1495, measured value 180.1499.

Step 3: 4-Chloro-5-nitro-1-(phenylsulfonyl)-1H-pyrrolo[2,3-b]pyridine (0.3 g, 0.9 mmol) was dissolved in 100 mL of tetrahydrofuran followed by adding DIPEA (0.7 g, 5.5 mmol) and (S)-4-(7-amino-5-azaspiro[2,4]hept-5-yl)butanenitrile (0.2 g, 1.1 mmol). Raise the temperature to maintain reflux and stir for 4 h. The reaction was monitored for completion by TLC. Concentrate under vacuum to yield a yellow oily liquid. Triturate and cure with an appropriate amount of methanol to yield (S)-4-(7-((5-nitro-1-(phenylsulfonyl)-1H-pyrrolo[2,3-b]pyridin-4-yl)amino)-5-azaspiro[2,4]hept-5-yl)butanenitrile as a yellow solid (0.3 g, 70%). The product can be used directly in the next step without further purification. HRMS (ESI): m/z [M+H]⁺. C₂₃H₂₅N₆O₄S calculated value 481.1653, measured value 481.1659.

Step 4: Dissolve (S)-4-(7-((5-nitro-1-(phenylsulfonyl)-1H-pyrrolo[2,3-b]pyridin-4-yl)amino)-5-azaspiro[2,4]hept-5-yl)butanenitrile (0.3 g, 0.6 mmol) in 10 mL of methanol, add palladium carbon (0.1 g, 10%) and replace the air in the reaction flask more than three times with hydrogen gas. The reaction was kept in hydrogen atmosphere and the reaction was stirred at room temperature for 12 h. The reaction was monitored for completion by TLC. The filtrate was collected and concentrated under vacuum to yield (S)-4-(7-(((5-amino-1-(phenylsulfonyl)-1H-pyrrolo[2,3-b]pyridin-4-yl)amino)-5-azaspiro[2,4]hept-5-yl)butanenitrile as a light pink foamy solid (0.3 g, 100%). The product can be used directly in the next step without further purification. HRMS (ESI): m/z [M+H]⁺. C₂₃H₂₇N₆O₂S calculated value 451.1911, measured value 451.1922.

Step 5: Triethyloxonium tetrafluoroborate (0.4 g, 2.0 mmol) and (R)-lactamide (0.2 g, 2.0 mmol) were dissolved in 20 mL of tetrahydrofuran, stirred at room temperature for 3 h and concentrated under vacuum to obtain the mixture as an oil, followed by the addition of 20 mL of ethanol to dissolve and the addition of (S)-4-(7-(((5-amino-1-(phenylsulfonyl)-1H-pyrrolo[2,3-b]pyridin-4-yl)amino)-5-azaspiro[2.4]hept-5-yl)butanenitrile (0.3 g, 0.7 mmol). Raise the temperature to maintain reflux and stir for 3 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum. The residue was purified using a silica gel column (petroleum ether:ethyl acetate=1:1) to yield 4-((S)-7-(2-((R)-1-Hydroxyethyl)-6-(phenylsulfonyl)imidazo[4,5-d]pyrrolo[2,3-b]pyridin-1(6H)-yl)-5-azaspiro[2,4] hept-5-yl)butanenitrile as a light yellow oil (0.2 g, 60%). HRMS (ESI): m/z [M+H]⁺. C₂₆H₂₉N₆O₃S calculated value 505.2016, measured value 505.2020.

Step 6: 4-((S)-7-(2-((R)-1-Hydroxyethyl)-6-(phenylsulfonyl)imidazo[4,5-d]pyrrolo[2,3-b]pyridin-1(6H)-yl)-5-azaspiro[2,4]hept-5-yl)butanenitrile (0.2 g, 0.4 mmol) was dissolved in a solvent mixture of 5 mL tetrahydrofuran and 5 mL methanol, followed by adding 5 mL of 1 M sodium hydroxide and then stirring at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate=1:1) to yield LXS69: 4-((S)-7-(2-((R)-1-Hydroxyethyl)imidazo[4,5-d]pyrrolo[2,3-b]pyridin-1(6H)-yl)-5-azaspiro[2,4]hept-5-yl)butanenitrile (0.1 g, 69%). ¹HNMR (300 MHz, DMSO-d₆): δ=11.96 (s, 1H), 8.79 (s, 1H), 7.59 (s, 1H), 6.85 (s, 1H), 4.58-4.71 (m, 1H), 3.70-3.77 (m, 1H), 2.66-2.83 (m, 2H), 2,47 (t, J=7.5 Hz, 2H), 2.12-2.22 (m, 2H), 1.88 (t, J=9.0 Hz, 2H), 1.76-1.80 (m, 2H), 1.48 (d, J=9.0 Hz, 3H), 0.05-0.27 (m, 4H) ppm; ¹³CNMR (75 MHz, DMSO-d₆) δ 149.33, 148.58, 141.66, 129.05, 127.99, 120.78, 119.38, 115.64, 99.56, 71.22, 68.47, 63.68, 56.19, 53.48, 25.59, 22.83, 15.98, 15.11, 4.22 ppm; HRMS (ESI): m/z [M+H]⁺. C₂₀H₂₅N₆O calculated value 365.2084, measured value 365.2089.

### Example 70

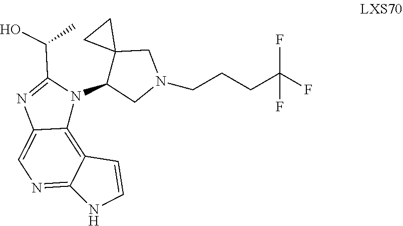 [SMILES: C[C@@H](O)c1nc2cnc3[nH]ccc3c2n1[C@@H]1CN(CCCC(F)(F)F)CC12CC2]

### (R)-1-(1-((S)-5-(4,4,4-Trifluorobutyl)-5-azaspiro[2,4]hept-7-yl)-1,6-dihydroimidazo[4,5-d]pyrrolo[2,3-b]pyridin-2-yl)ethanol

LXS70 was prepared in a similar preparation way to Example 69 and the crude product was purified using a silica gel column (petroleum ether:ethyl acetate=1:1) to yield LXS70: (R)-1-(1-((S)-5-(4,4,4-Trifluorobutyl)-5-azaspiro[2,4]hept-7-yl)-1,6-dihydroimidazo[4,5-d]pyrrolo[2,3-b]pyridin-2-yl) ethanol (0.1 g, 66%). ¹HNMR (300 MHz, DMSO-d₆): δ=12.03 (s, 1H), 8.77 (s, 1H), 7.61 (s, 1H), 6.93 (s, 1H), 4.60-4.73 (m, 1H), 3.73-3.79 (m, 1H), 2.56-2.81 (m, 2H), 2.43 (t, J=9.0 Hz, 2H), 2.15-2.26 (m, 2H), 1.77-1.81 (m, 2H), 1.43 (d, J=4.5 Hz, 3H), 1.26-1.38 (m, 2H), 0.02-0.22 (m, 4H) ppm; ¹³CNMR (75 MHz, DMSO-d₆) δ 148.93, 148.51, 142.16, 129.04, 127.11, 126.83, 120.77, 115.61, 99.78, 71.21, 67.54, 62.59, 56.93, 53.76, 37.74, 25.78, 22.81, 10.16, 5.83 ppm; HRMS (EST): m/z [M+H]⁺. C₂₀H₂₅F₃N₅O calculated value 408.2006, measured value 408.2010.

### Example 71

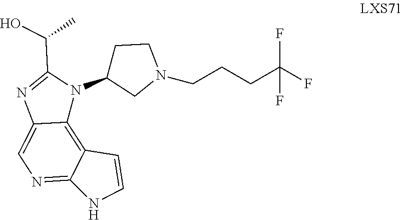 [SMILES: C[C@@H](O)c1nc2cnc3[nH]ccc3c2n1[C@H]1CCN(CCCC(F)(F)F)C1]

### (R)-1-(1-((S)-1-(4,4,4-Trifluorobutyl)pyrrolidin-3-yl)-1,6-dihydroimidazo[4,5-d]pyrrolo[2,3-b]pyridin-2-yl)ethanol

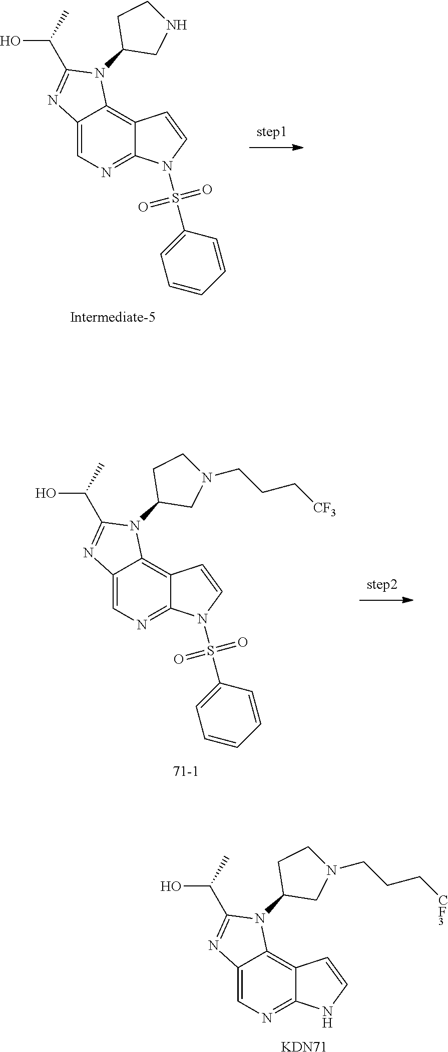

Step 1: Dissolve (R)-1-(6-(phenylsulfonyl)-1-((S-pyrrolidin-3-yl)-1,6-dihydroimidazo[4,5-d]pyrrolo[2,3-b]pyridin-2-yl) ethanol (0.3 g, 0.7 mmol) in 10 mL of tetrahydrofuran, add DIPEA (0.2 g, 1.4 mmol) followed by slow dropwise addition of 4,4,4-trifluoro-1-iodobutane (0.3 g, 1.1 mmol). Raise the temperature to maintain reflux and stir for 3 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield (R)-1-(6-(phenylsulfonyl)-1-((S)-1-(4,4,4-trifluorobutyl)pyrrolidin-3-yl)-1,6-dihydroimidazo[4,5-d]pyrrolo[2,3-b]pyridin-2-yl) ethanol as a pale yellow oil (0.3 g, 79%). The product can be used directly in the next step without further purification. HRMS (EST): m/z [M+H]⁺. C₂₄H₂₇F₃N₅O₃S calculated value 522.1781, measured value 522.1788.

Step 2: (R)-1-(6-(phenylsulfonyl)-1-((S)-1-(4,4,4-trifluorobutyl)pyrrolidin-3-yl)-1,6-dihydroimidazo[4,5-d] pyrrolo[2,3-b]pyridin-2-yl) ethanol (0.3 g, 0.6 mmol) was dissolved in a solvent mixture of 5 mL tetrahydrofuran and 5 mL methanol, and 5 mL of lM sodium hydroxide was added and then stirred at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhvdrous sodium sulfate and concentrated under vacuum, and the residue was purified using a silica gel column (petroleum ether:ethyl acetate=1:1) to yield LXS71: (R)-1-(1-((S)-1-(4,4,4-trifluorobutyl)pyrrolidin-3-yl)-1,6-dihydroimidazo[4,5-d]pyrrolo[2,3-b]pyridin-2-yl)ethanol (0.1 g, 46%). ¹HNMR (300 MHz, DMSO-d₆): δ=12.04 (s, 1H), 8.83 (s, 1H), 7.56 (s, 1H), 6.89 (s, 1H), 4.61-4.74 (m, 1H), 3.71-3.79 (m, 1H), 2.56-2.81 (m, 2H), 2.41 (t, J=4.5 Hz, 2H), 2.20-2.32 (m, 2H), 1.90-2.15 (m, 2H), 1.80-1.87 (m, 2H), 1.44 (d, J=6.0 Hz, 3H), 1.35-1.40 (m, 2H) ppm; ¹³CNMR (75 MHz, DMSO-d₆) δ 150.11, 149.33, 144.34, 129.88, 127.19, 126.53, 120.77, 116.53, 98.57, 64.78, 58.93, 58.42, 56.32, 55.93, 37.77, 27.61, 22.84, 10.56 ppm; HRMS (ESI): m/z [M+H]⁺. C₁₈H₂₃F₃N₅O calculated value 382.1849, measured value 382.1852.

### Example 72

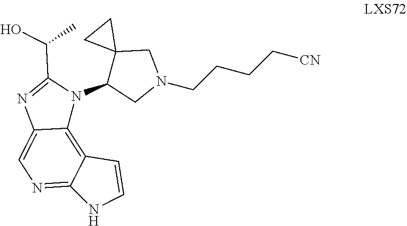 [SMILES: C[C@@H](O)c1nc2cnc3[nH]ccc3c2n1[C@@H]1CN(CCCCC#N)CC12CC2]

### 5-((S)-7-(2-((R)-1-Hydroxyethyl)imidazo[4,5-d]pyrrolo[2,3-b]pyridin-1(6H)-yl)-5-azaspiro[2,4]hept-5-yl)pentanonitrile

LXS72 was prepared in a similar preparation way to Example 69 and the crude product was purified using a silica gel column (petroleum ether:ethyl acetate=1:1) to yield LXS72: 5-((S)-7-(2-((R)-1-Hydroxyethyl)imidazo[4,5-d]pyrrolo[2,3-b]pyridin-1(6H)-yl)-5-azaspiro[2,4]hept-5-yl)pentanonitrile (0.1 g, 45%). ¹HNMR (300 MHz, DMSO-d₆): δ=11.99 (s, 1H), 8.87 (s, 11H), 7.59 (s, 1H), 6.72 (s, 1H), 4.63-4.77 (m, 11H), 3.73-3.82 (m, 11H), 2.52-2.74 (m, 2H), 2.47 (t, J=6.0 Hz, 2H), 2.12-2.23 (m, 2H), 1.87 (t, J=6.0 Hz, 2H), 1.59-1.68 (m, 2H), 1.48 (d, J=9.0 Hz, 3H), 1.33-1.41 (m, 2H), 0.08-0.22 (m, 4H) ppm; ¹³CNMR (75 MHz, DMSO-d₆) δ 150.68, 149.47, 142.19, 129.33, 127.86, 120.73, 119.39, 115.62, 98.56, 71.29, 68.40, 65.27, 56.44, 53.49, 27.28, 25.58, 23.29, 22.81, 17.11, 5.88 ppm; HRMS (ESI): m/z [M+H]⁺. C₂₁H₂₇N₆O calculated value 379.2241, measured value 379.2243.

### Example 73

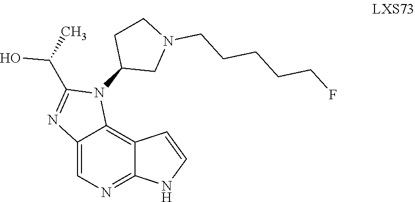 [SMILES: C[C@@H](O)c1nc2cnc3[nH]ccc3c2n1[C@H]1CCN(CCCCCF)C1]

### (R)-1-(1-((S)-1-(5—Fluoropentyl)pyrrolidin-3-yl)-1,6-dihydroimidazo[4,5-d]pyrrolo[2,3-b]pyridin-2-yl)ethanol

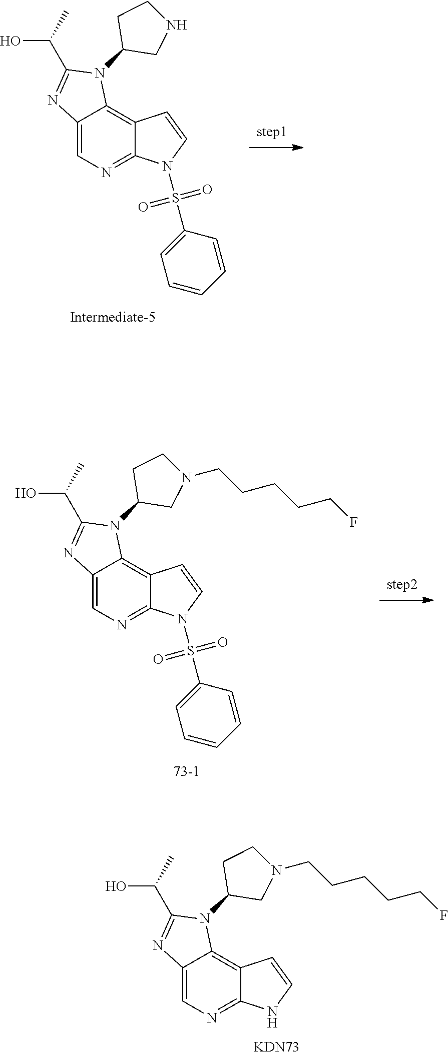

Step 1: Dissolve (R)-1-(6-(phenylsulfonyl)-1-((S-pyrrolidin-3-yl)-1,6-dihydroimidazo[4,5-d]pyrrolo[2,3-b]pyridin-2-yl)ethanol (0.3 g, 0.7 mmol) in 10 mL of tetrahydrofuran, add DIPEA (0.2 g, 1.4 mmol) followed by slow dropwise addition of 1-bromo-5-fluoropentane (0.2 g, 1.1 mmol), raise the temperature to reflux and stir for 3 h. Monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield (R)-1-(1-((S)-1-(5-fluoropentyl)pyrrolidin-3-yl)-6-(phenylsulfonyl)-1,6-dihydroimidazo[4,5-d]pyrrolo[2,3-b]pyridin-2-yl)ethanol as a pale yellow oil (0.3 g, 82%). The product can be used directly in the next step without further purification. HRMS (ESI): m/z [M+H]⁺. C₂₅H₃₁FN₅O₃S calculated value 500.2126, measured value 500.2130.

Step 2: (R)-1-(1-((S)-1-(5—Fluoropentyl)pyrrolidin-3-yl)-6-(phenylsulfonyl)-1,6-dihydroimidazo[4,5-d]pyrrolo[2,3-b]pyridin-2-yl) ethanol (0.3 g, 0.6 mmol) was dissolved in a mixture of 5 mL of tetrahydrofuran and 5 mL of methanol, and 5 mL of 1 M sodium hydroxide was added, then stir for 5 h at room temperature, the reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum. The residue was purified using a silica gel column (petroleum ether:ethyl acetate=1:1) to yield LXS73: (R)-1-(1-((S)-1-(5-fluoropentyl)pyrrolidin-3-yl)-1,6-dihydroimidazo[4,5-d]pyrrolo[2,3-b]pyridin-2-yl) ethanol (0.1 g, 46%). ¹HNMR (300 MHz, DMSO-d₆): δ=11.98 (s, 1H), 8.88 (s, 1H), 7.63 (s, 1H), 6.84 (s, 1H), 4.68-4.76 (m, 1H), 4.09-4.13 (m, 2H), 3.74-3.79 (m, 1H), 2.59-2.84 (m, 2H), 2.49 (t, J=9.0 Hz, 2H), 2.19-2.30 (m, 2H), 1.91-2.18 (m, 2H), 1.49 (d, J=9.0 Hz, 3H), 1.40-1.45 (m, 2H), 1.36-1.40 (m, 2H), 1.27-1.31 (m, 2H) ppm; ¹³CNMR (75 MHz, DMSO-d₆) δ 151.08, 148.53, 142.19, 129.04, 127.88, 120.78, 115.62, 98.67, 83.69, 64.55, 58.92, 58.44, 56.61, 55.92, 30.71, 28.09, 27.66, 19.45 ppm; HRMS (ESI): m/z [M+H]. C₁₉H₂₇FN₅O calculated value 360.2194, measured value 360.2199.

### Example 74

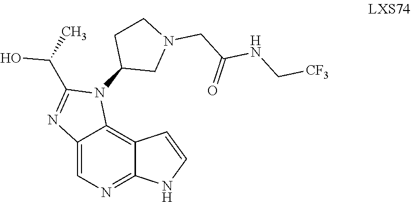 [SMILES: C[C@@H](O)c1nc2cnc3[nH]ccc3c2n1[C@H]1CCN(CC(=O)NCC(F)(F)F)C1]

### 2-((S)-3-(2-((R)-1-Hydroxyethyl)imidazo[4,5-d]pyrrolo[2,3-b]pyridin-1(6H)-yl)pyrrolidin-1-yl)-N-(2,2,2-trifluoroethyl)acetamide

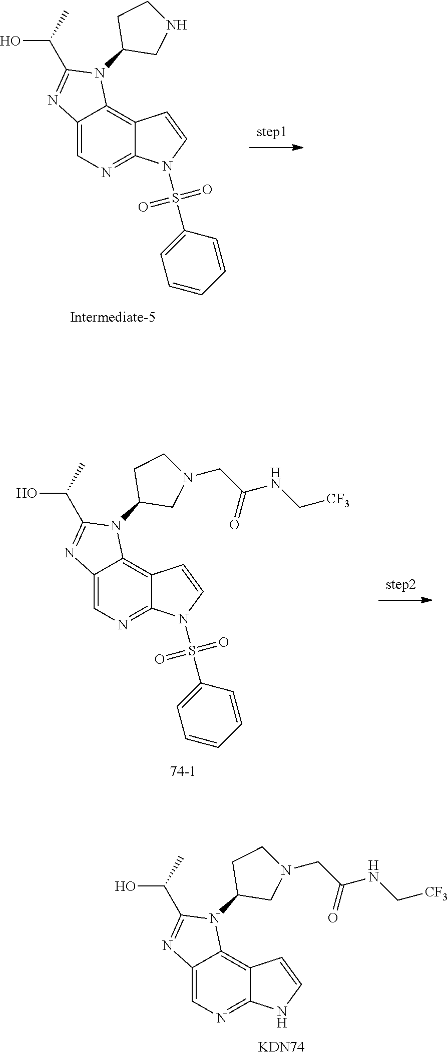

Step 1: Dissolve (R)-1-(6-(phenylsulfonyl)-1-((S-pyrrolidin-3-yl)-1,6-dihydroimidazo[4,5-d]pyrrolo[2,3-b]pyridin-2-yl)ethanol (0.3 g, 0.7 mmol) in 10 mL of tetrahydrofuran, add DIPEA (0.2 g, 1.4 mmol) followed by slow dropwise addition of 2-bromo-N-(2,2,2-trifluoroethyl)acetamide (0.3 g, 1.1 mmol). Raise the temperature to reflux and stir for 3 h. Monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield 2-((S)-3-(2-((R)-1-Hydroxyethyl)-6-(phenylsulfonyl)imidazo[4,5-d]pyrrolo[2,3-b]pyridin-1(6H)-yl)pyrrolidin-1-yl)-N-(2,2,2-trifluoroethyl)acetamide as a pale yellow oil (0.3 g, 75%). The product can be used directly in the next step without further purification. HRMS (ESI): m/z [M+H]⁺. C₂₄H²⁶F₃N₆O₄S calculated value 551.1683, measured value 552.1688.

Step 2: Dissolve 2-((S)-3-(2-((R)-1-hydroxyethyl)-6-(phenylsulfonyl)imidazo[4,5-d]pyrrolo[2,3-b]pyridin-1(6H)-yl)pyrrolidin-1-yl)-N-(2,2,2-trifluoroethyl)acetamide (0.3 g, 0.5 mmol) in 5 mL of a mixture of tetrahydrofuran and 5 mL of methanol. Add 5 mL of 1 M sodium hydroxide and then stir at room temperature for 5 h. Monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS74: 2-((S)-3-(2-((R)-1-Hydroxyethyl)imidazo[4,5-d]pyrrolo[2,3-b]pyridin-1(6H)-yl)pyrrolidin-1-yl)-N-(2,2,2-trifluoroethyl)acetamide (0.1 g, 45%). ¹HNMR (300 MHz, DMSO-d₆): δ=12.11 (s, 1H), 8.89 (s, 1H), 8.03 (s, 1H), 7.58 (s, 1H), 6.91 (s, 1H), 4.65-4.78 (m, 1H), 3.72-3.79 (m, 3H), 3.25 (s, 2H), 2.58-2.83 (m, 2H), 2.21-2.34 (m, 2H), 1.94-2.18 (m, 2H), 1.49 (d, J=9.0 Hz, 3H) ppm; ¹³CNMR (75 MHz, DMSO-d₆) δ 170.55, 152.67, 148.55, 143.18, 129.04, 127.84, 124.79, 120.76, 115.69, 98.47, 64.87, 59.54, 58.28, 57.48, 54.94, 39.41, 27.88, 22.81 ppm: HRMS (ESI): m/z [M+H]⁺. C₁₈H₂₂F₃N₆O₂ calculated value 411.1751, measured value 411.1777.

### Example 75

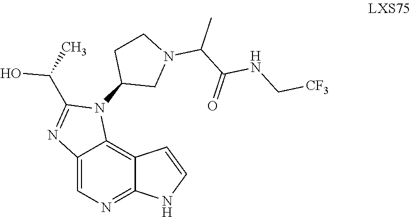 [SMILES: CC(C(=O)NCC(F)(F)F)N1CC[C@H](n2c([C@@H](C)O)nc3cnc4[nH]ccc4c32)C1]

### 2-((S)-3-(2-((R)-1-Hydroxyethyl)imidazo[4,5-d]pyrrolo[2,3-b]pyridin-1(6H)-yl)pyrrolidin-1-yl)-N-(2,2,2-trifluoroethyl)propanamide

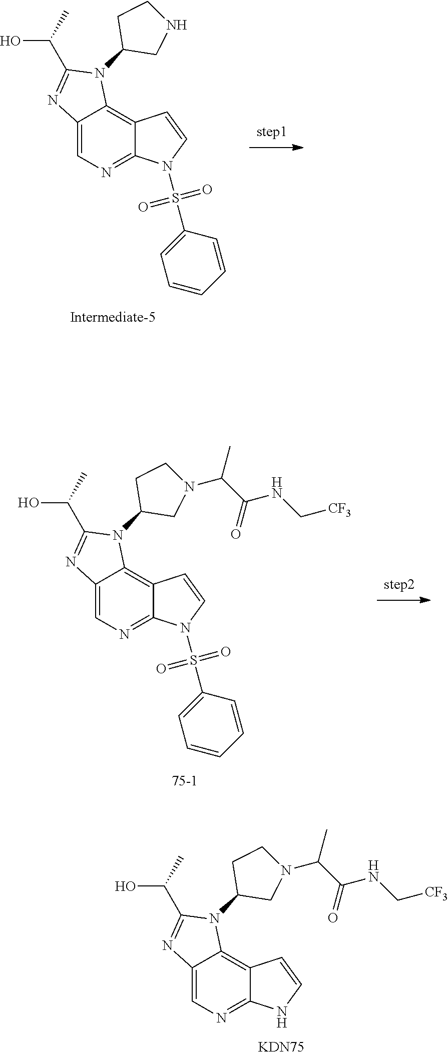

Step 1: Dissolve (R)-1-(6-(phenylsulfonyl)-1-((S-pyrrolidin-3-yl)-1,6-dihydroimidazo[4,5-d]pyrrolo[2,3-b]pyridin-2-yl)ethanol (0.3 g, 0.7 mmol) in 10 mL of tetrahydrofuran. Add DIPEA (0.2 g, 1.4 mmol) and then slowly add 2-bromo-N-(2,2,2-trifluoroethyl)propanamide (0.3 g, 1.1 mmol) dropwise, raise the temperature to reflux and stir for 3 h. Monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield 2-((S)-3-(2-((R)-1-hydroxyethyl)-6-(phenylsulfonyl)imidazo[4,5-d]pyrrolo[2,3-b]pyridin-1(6H)-yl)pyrrolidin-1-yl)-N-(2,2,2-trifluoroethyl)propanamide as a pale yellow oil (0.3 g, 73%). The product can be used directly in the next step without further purification. HRMS (ESI): m/z [M+H]⁺. C₂₅H₂₈F₃N₆O₄S calculated value 565.1839, measured value 565.1841.

Step 2. Dissolve 2-((S)-3-(2-((R)-1-hydroxyethyl)-6-(phenylsulfonyl)imidazo[4,5-d]pyrrolo[2,3-b]pyridin-1(6H)-yl)pyrrolidin-1-yl)-N-(2,2,2-trifluoroethyl)propanamide (0.3 g, 0.5 mmol) in a mixture of 5 mL of tetrahydrofuran and 5 mL of methanol. Add 5 mL of 1 M sodium hydroxide and stir at room temperature for 5 h. Monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS75: 2-((S)-3-(2-((R)-1-Hydroxyethyl)imidazo[4,5-d]pyrrolo[2,3-b]pyridin-1(6H)-yl)pyrrolidin-1-yl)-N-(2,2,2-trifluoroethyl)propanamide (0.1 g, 44%). ¹HNMR (300 MHz, DMSO-d₆): δ=11.91 (s, 1H), 8.77 (s, 1H), 8.05 (s, 1H), 7.68 (s, 1H), 6.86 (s, 1H), 4.67-4.79 (m, 1H), 3.65-3.79 (m, 4H), 2.56-2.81 (m, 2H), 2.18-2.31 (m, 2H), 1.90-2.15 (m, 2H), 1.41 (d, J=7.5 Hz, 3H), 1.28 (d, J=7.5 Hz, 3H) ppm; ¹³ CNMR (75 MHz, DMSO-d₆) δ 171.96, 148.67, 148.51, 142.18, 129.04, 127.18, 124.75, 120.75, 115.64, 97.56, 69.29, 63.64, 58.59, 54.92, 52.48, 39.47, 27.36, 22.86, 18.44 ppm, HRMS (EST): m/z [M+H]⁺. C₁₉H₂₄F₃N₆O₂ calculated value 425.1907, measured value 425.1911.

### Example 76

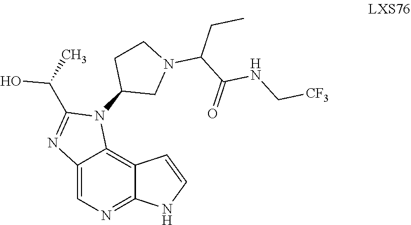 [SMILES: CCC(C(=O)NCC(F)(F)F)N1CC[C@H](n2c([C@@H](C)O)nc3cnc4[nH]ccc4c32)C1]

### 2-((S)-3-(2-((R)-1-Hydroxyethyl)imidazo[4,5-d]pyrrolo[2,3-b]pyridin-1(6H)-yl)pyrrolidin-1-yl)-N-(2,2,2-trifluoroethyl)butanamide

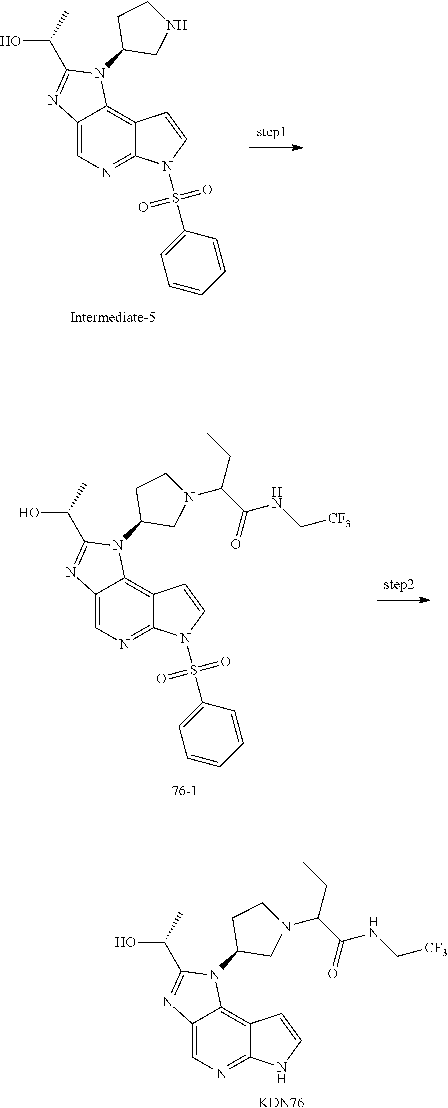

Step 1: Dissolve (R)-1-(6-(phenylsulfonyl)-1-((S-pyrrolidin-3-yl)-1,6-dihydroimidazo[4,5-d]pyrrolo[2,3-b]pyridin-2-yl)ethanol (0.3 g, 0.7 mmol) in 10 mL of tetrahydrofuran, add DIPEA (0.2 g, 1.4 mmol) followed by the slow dropwise addition of 2-bromo-N-(2,2,2-trifluoroethyl)butanamide (0.3 g, 1.1 mmol). Raise the temperature to maintain reflux and stir for 3 h. Monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield 2-((S)-3-(2-((R)-1-hydroxyethyl)-6-(phenylsulfonyl)imidazo[4,5-d]pyrrolo[2,3-b]pyridin-1(6H)-yl)pyrrolidin-1-yl)-N-(2,2,2-trifluoroethyl)butanamide as a pale yellow oil (0.3 g, 71%). The product can be used directly in the next step without further purification. HRMS (ESI): m/z [M+H]⁺. C₂₆H₃₀F₃N₆O₄S calculated value 579.1996, measured value 579.2001.

Step 2: Dissolve 2-((S)-3-(2-((R)-1-hydroxyethyl)-6-(phenylsulfonyl)imidazo[4,5-d]pyrrolo[2,3-b]pyridin-1(6H)-yl)pyrrolidin-1-yl)-N-(2,2,2-trifluoroethyl)butanamide (0.3 g, 0.5 mmol) in a mixture of 5 mL tetrahydrofuran and 5 mL methanol. Add 5 mL of 1M sodium hydroxide and stir at room temperature for 5 h. Monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate=1:1) to yield LXS76: 2-((S)-3-(2-((R)-1-Hydroxyethyl)imidazo[4,5-d]pyrrolo[2,3-b]pyridin-1(6H)-yl)pyrrolidin-1-yl)-N-(2,2,2-trifluoroethyl)butanamide (0.1 g, 44%). ¹HNMR (300 MHz, DMSO-d₆): δ=12.01 (s, 1H), 8.78 (s, 1H), 8.08 (s, 1H), 7.53 (s, 1H), 6.79 (s, 1H), 4.68-4.74 (m, 1H), 3.56-3.79 (m, 4H), 2.57-2.85 (m, 2H), 2.20-2.30 (m, 2H), 1.93-2.17 (m, 2H), 1.57-1.64 (m, 2H), 1.48 (d, J=7.5 Hz, 3H), 0.91 (t, J=9.0 Hz, 3H) ppm; ¹³CNMR (75 MHz, DMSO-d₆) δ 173.88, 151.77, 148.53, 142.11, 129.04, 127.17, 124.77, 120.71, 115.63, 98.56, 75.93, 64.65, 58.53, 55.22, 52.71, 39.48, 27.63, 23.66, 22.84, 12.89 ppm; HRMS (ESI): m/z [M+H]⁺. C₂₀H₂₆F₃N₆O₂ calculated value 439.2064, measured value 439.2072.

### Example 77

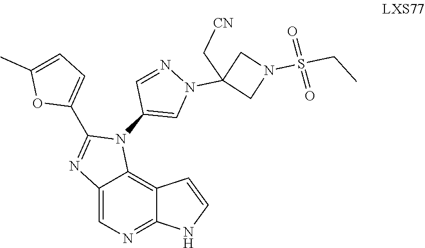

### 2-(1-(Ethylsulfonyl)-3-(4-(2-(5-methylfuran-2-yl)imidazolyl[4,5-d]pvrrolo[2,3-b]pyridin-1(6H)-yl)-1H-pyrazol-1-yl)azetidin-3-yl)acetonitrile

LXS77 was prepared using a similar preparation way to Example 63 and the crude product was purified using a silica gel column (petroleum ether:ethyl acetate=1:1) to yield LXS77: 2-(1-(ethylsulfonyl)-3-(4-(2-(5-methylfuran-2-yl)imidazolyl[4,5-d]pyrrolo[2,3-b]pyridin-1(6H)-yl)-1H-pyrazol-1-yl) azetidin-3-yl) acetonitrile (0.1 g, 53%). ¹HNMR (300 MHz, DMSO-d₆): δ=12.07 (s, 1H), 8.88 (s, 1H), 8.08 (s, 2H), 7.58 (s, 1H), 6.95 (d, J=7.5 Hz, 1H), 6.83 (s, 1H), 6.08 (d, J=7.5 Hz, 1H), 4.80-4.14 (m, 4H), 3.45 (t, J=9.0 Hz, 2H), 2.85 (s, 2H), 2.30 (s, 3H), 1.22 (t, J=7.5 Hz, 3H) ppm; ¹³CNMR (75 MHz, DMSO-d₆) δ 152.28, 151.49, 149.11, 143.19, 141.55, 130.75, 129.78, 129.01, 128.66, 121.67, 117.78, 115.61, 107.81, 107.62, 100.57, 99.56, 59.27, 51.63, 50.83, 23.27, 15.98, 4.77 ppm. HRMS (ESI): m/z [M+H]⁺. C₂H₂₃N₅O₃S calculated value 491.1608, measured value 491.1612.

### Example 78

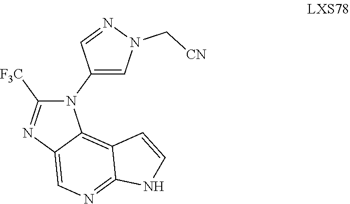 [SMILES: N#CCn1cc(-n2c(C(F)(F)F)nc3cnc4[nH]ccc4c32)cn1]

### 2-(4-(2-(Trifluoromethyl)imidazo[4,5-d]pyrrolo[2,3-b]pyridin-1(6H)-yl)-1H-pyrazol-1-yl)acetonitrile

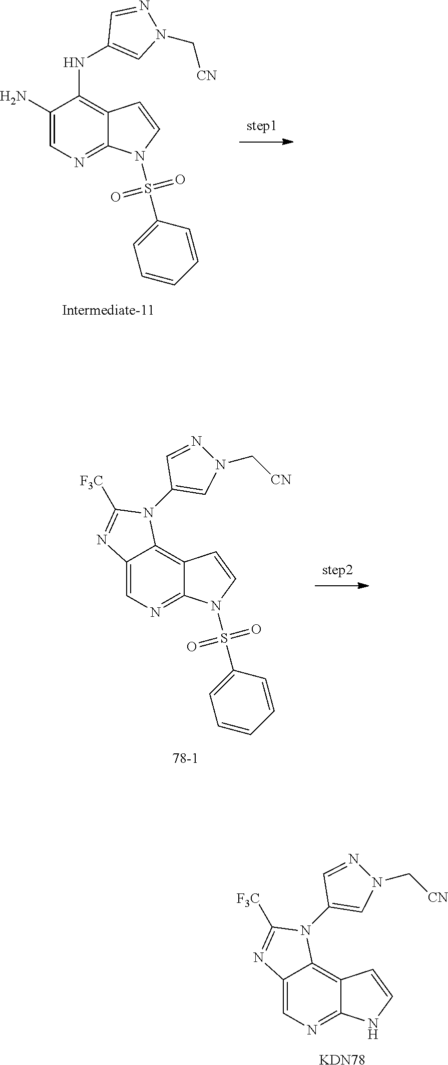

Step 1: Triethyloxonium tetrafluoroborate (0.5 g, 2.4 mmol) and 2,2,2-trifluoroacetamide (0.2 g, 2.4 mmol) were dissolved in 10 mL of tetrahydrofuran, stirred at room temperature for 3 h and concentrated under vacuum to obtain the mixture in the form of oil, followed by the addition of 10 mL of ethanol to dissolve and the addition of 2-(4-((5-amino-1-(phenylsulfonyl)-1H-pyrrolo[2,3-b]pyridin-4-yl)amino)-1H-pyrazol-1-yl)acetonitrile (0.3 g 0.8 mmol). Raise the temperature to maintain reflux and stir for 3 h. The reaction was monitored by TLC for complete reaction. Saturated sodium bicarbonate was added until the reaction solution was weakly basic, and the organic phase was separated. The aqueous phase was extracted twice using dichloromethane. The organic phases were combined and washed with saturated saline, dried over anhvdrous sodium sulfate and concentrated under vacuum to yield 2-(4-(6-(phenylsulfonyl)-2-(trifluoromethyl)imidazo[4,5-d]pyrrolo[2,3-b]pyridin-1(6H)-yl)-1H-pyrazol-1-yl) acetonitrile as a milk white oil (0.3 g, 83%). HRMS (ESI): m/z [M+H]⁺. C₂₀H₁₃F₃N₇O₂S calculated value 472.0798, measured value 472.0802.

Step 2: Dissolve 2-(4-(6-(phenylsulfonyl)-2-(trifluoromethyl)imidazo[4,5-d]pyrrolo[2,3-b]pyridin-1(6H)-yl)-1H-pyrazol-1-yl)acetonitrile (0.3 g, 0.6 mmol) in a solvent mixture of 5 mL tetrahydrofuran and 5 mL methanol, and stir at room temperature for 5 h after adding 5 mL of 1 M sodium hydroxide. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS78: 2-(4-(2-(trifluoromethyl)imidazo[4,5-d]pyrrolo[2,3-b]pyridin-1(6H)-yl)-1H-pyrazol-1-yl)acetonitrile (0.1 g, 47%). ¹HNMR (300 MHz, DMSO-d₆): δ=11.97 (s, 1H), 8.87 (s, 1H), 8.05 (s, 1H), 7.91 (s, 1H), 7.56 (s, 1H), 6.83 (s, 1H), 4.85 (s, 2H) ppm; ¹³CNMR (75 MHz, DMSO-d₆) δ 148.11, 144.33, 130.74, 129.74, 129.04, 127.65, 120.73, 116.73, 116.32, 115.62, 100.53, 98.45, 45.65 ppm; HRMS (ESI): m/z [M+H]⁺. C₁₋₄H₉F₃N₇ calculated value 332.0866, measured value 332.0871.

### Example 79

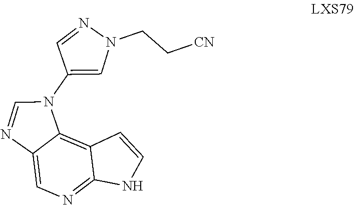 [SMILES: N#CCCn1cc(-n2cnc3cnc4[nH]ccc4c32)cn1]

### 3-(4-(Imidazo[4,5-d]pyrrolo[2,3-b]pyridin-1(6H)-yl)-1H-pyrazol-1-yl)propanenitrile

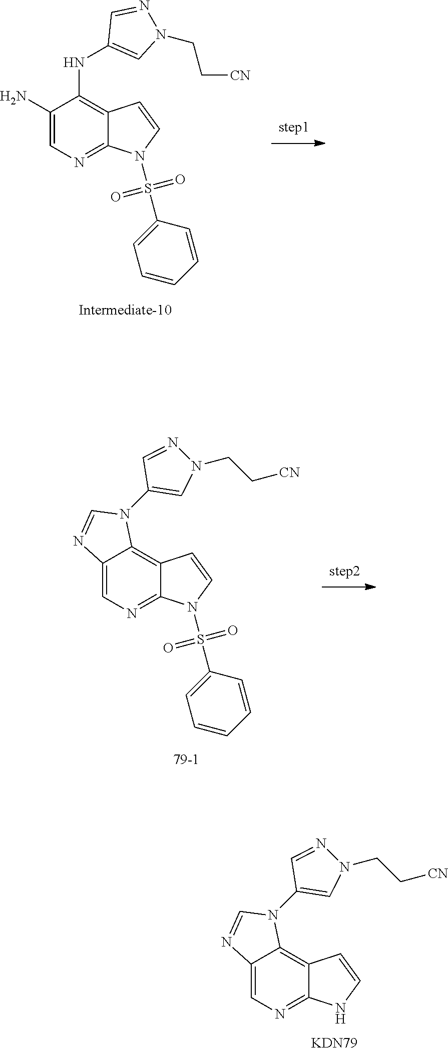

Step 1: Dissolve 3-(4-((5-amino-1-(phenylsulfonyl)-1H-pyrrolo[2,3-b]pyridin-4-yl)amino)-1H-pyrazol-1-yl)propanenitrile (0.3 g, 0.7 mmol) in 10 mL of toluene, followed by the addition of triethyl orthoformate (0.2 g, 1.4 mmol), p-toluenesulfonic acid (30.0 mg, 10%), and raise the temperature to maintain reflux and stir for 3 h. The reaction was monitored for completion by TLC. Saturated sodium bicarbonate was added until the reaction solution was weakly basic, and the organic phase was separated. The aqueous phase was extracted twice with dichloromethane. The organic phases were combined and washed with saturated saline, dried over anhydrous sodium sulfate and concentrated under vacuum to yield 3-(4-(6-(phenylsulfonyl)imidazo[4,5-d]pyrrolo[2,3-b]pyridin-1(6H)-yl)-1H-pyrazol-1-yl)propanenitrile as a pale yellow oil (0.3 g, 98%). HRMS (ESI): m/z [M+H]⁺. C₂₀H₁₆N₇O₂S calculated value 418.1081, measured value 418.1088.

Step 2: Dissolve 3-(4-(6-(phenylsulfonyl)imidazo[4,5-d]pyrrolo[2,3-b]pyridin-1(6H)-yl)-1H-pyrazol-1-yl)propanenitrile (0.3 g, 0.7 mmol) in a solvent mixture of 5 mL tetrahydrofuran and 5 mL methanol, add 5 mL of 1 M sodium hydroxide and then stir at room temperature for 5 h. Monitor the reaction by TLC for completion. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS79: 3-(4-(imidazo[4,5-d]pyrrolo[2,3-b]pyridin-1(6H)-yl)-1H-pyrazol-1-yl)propanenitrile (0.1 g, 50%). ¹HNMR (300 MHz, DMSO-d₆): δ=11.99 (s, 1H), 8.89 (s, 1H), 8.15 (s, 1H), 7.95 (s, 1H), 7.56 (s, 1H), 7.15 (s, 1H), 6.89 (s, 1H), 5.04 (t, J=7.5 Hz, 2H), 3.21 (t, J=7.5 Hz, 2H) ppm; ¹³CNMR (75 MHz, DMSO-d₆) δ 150.44, 142.88, 134.58, 130.74, 129.73, 129.04, 127.16, 120.74, 117.73, 115.60, 100.54, 98.62, 49.37, 17.50 ppm; HRMS (ESI): m/z [M+H]⁺. C₁₄H₁₂N₇ calculated value 278.1149, measured value 278.1151.

### Example 80

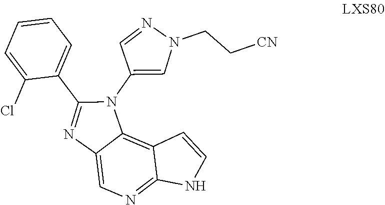 [SMILES: N#CCCn1cc(-n2c(-c3ccccc3Cl)nc3cnc4[nH]ccc4c32)cn1]

### 3-(4-(2-(2-Chlorophenyl)imidazo[4,5-d]pyrrolo[2,3-b]pyridin-1(6H)-yl)-1H-pyrazol-1-yl)propanenitrile

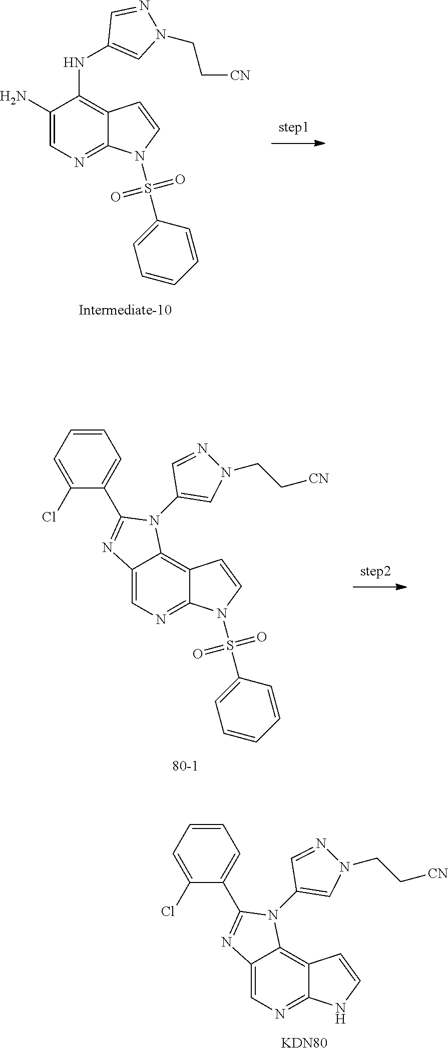

Step 1: Dissolve 3-(4-((5-amino-1-(phenylsulfonyl)-1H-pyrrolo[2,3-b]pyridin-4-yl)amino)-1H-pyrazol-1-yl)propanenitrile (0.3 g, 0.7 mmol) in 10 mL of DMF, add Na₂S₂O₅ (0.7 g, 3.5 mmol) followed by the addition of 2-chlorobenzaldehyde (0.2 g, 1.0 mmol). Stir for 12 h at 90° C. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline and dried over anhydrous sodium sulfate to yield 3-(4-(2-(2-chlorophenyl)-6-(phenylsulfonyl)imidazo[4,5-d]pyrrolo[2,3-b]pyridin-1(6H)-yl)-1H-pyrazol-1-yl)propanenitrile as a yellow solid (0.3 g, 77%). HRMS (ESI): m/z [M+H]⁺. C₂₆H₁₉ClN₇O₂S calculated value 528.1004, measured value 528.1014.

Step 2: Dissolve 3-(4-(2-(2-chlorophenyl)-6-(phenylsulfonyl)imidazo[4,5-d]pyrrolo[2,3-b]pyridin-1(6H)-yl)-1H-pyrazol-1-yl)propanenitrile (0.3 g, 0.6 mmol) in a mixture of 5 mL tetrahydrofuran and 5 mL methanol, add 5 mL of 1 M sodium hydroxide and then stir at room temperature for 5 hours. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate=1:1) to yield LXS80: 3-(4-(2-(2-Chlorophenyl)imidazo[4,5-d]pyrrolo[2,3-b]pyridin-1(6H)-yl)-1H-pyrazol-1-yl)propanenitrile (0.1 g, 45%). ¹HNMR (300 MHz, DMSO-d₆): δ=12.03 (s, 1H), 8.78 (s, 1H), 8.08 (s, 1H), 7.99 (s, 1H), 7.73 (d, J=7.5 Hz, 1H), 7.58 (s, 1H), 7.55 (d, J=7.5 Hz, 1H), 7.35-7.39 (m, 2H), 6.85 (s, 1H), 5.09 (t, J=9.0 Hz, 2H), 3.28 (t, J=9.0 Hz, 2H) ppm: ¹′CNMR (75 MHz, DMSO-d₆) δ 149.21, 143.48, 142.19, 138.56, 132.27, 130.77, 130.16, 129.75, 129.38, 129.05.127.30, 127.17, 120.77, 117.74, 115.64, 100.63, 98.56, 50.45, 17.54 ppm: HRMS (ESI): m/z [M+H]⁺. C₂₀H₁₅ClN₇ calculated value 388.1072, measured value 388.1077.

### Example 81

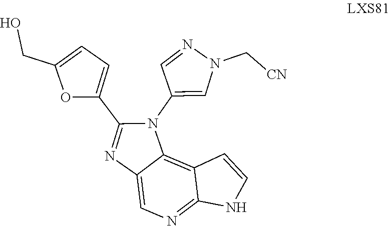 [SMILES: N#CCn1cc(-n2c(-c3ccc(CO)o3)nc3cnc4[nH]ccc4c32)cn1]

### 2-(4-(2-(5-(Hydroxymethyl)furan-2-yl)imidazo[4,5-d]pyrrolo[2,3-b]pyridin-1(6H)-yl)-1H-pyrazol-1-yl)acetonitrile

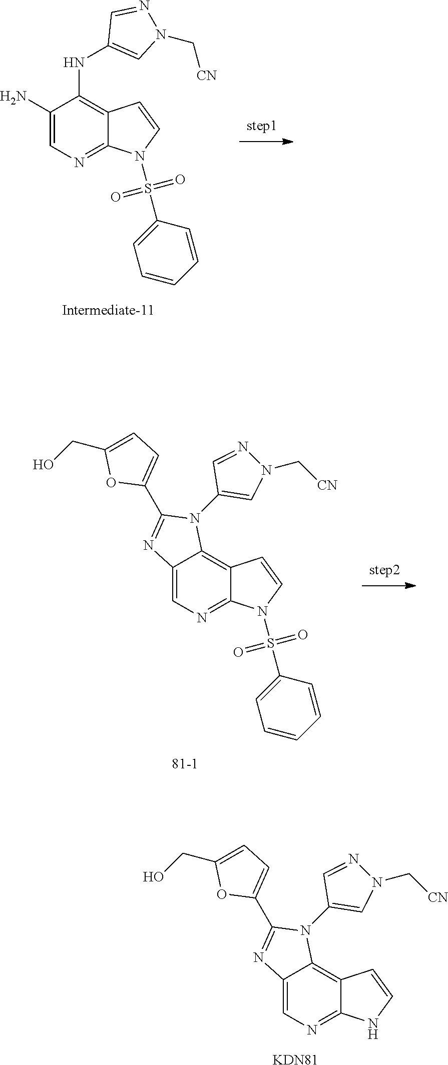

Step 1: Dissolve 2-(4-((5-amino-1-(phenylsulfonyl)-1H-pyrrolo[2,3-b]pyridin-4-yl)amino)-1H-pyrazol-1-yl)acetonitrile (0.3 g, 0.8 mmol) in 10 mL of DMF, add Na₂S₂O₅ (0.8 g, 4.0 mmol) followed by adding 5-hydroxymethylfurfural (0.3 g, 1.6 mmol). Stir for 12 h at 90° C. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield 2-(4-(2-(5-(hydroxymethyl)furan-2-yl)-6-(phenylsulfonyl)imidazo[4,5-d]pyrrolo[2,3-b]pyridin-1(6H)-yl)-1H-pyrazol-1-yl) acetonitrile as a pale yellow oil (0.3 g, 79%). HRMS (EST): m/z [M+H]⁺. C₂₄H₁₈N₇O₄S calculated value 500.1135, measured value 500.1140.

Step 2: Dissolve 2-(4-(2-(5-(hydroxymethyl)furan-2-yl)-6-(phenylsulfonyl)imidazo[4,5-d]pyrrolo[2,3-b]pyridin-(6H)-yl)-1H-pyrazol-1-yl)acetonitrile (0.3 g, 0.6 mmol) in a mixture of 5 mL of tetrahydrofuran and 5 mL of methanol, then add 5 mL of 1 M sodium hydroxide. Stir for 5 h at room temperature. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum. The residue was purified using a silica gel column (petroleum ether:ethyl acetate=1:1) to yield LXS81: 2-(4-(2-(5-(Hydroxymethyl)furan-2-yl)imidazo[4,5-d]pyrrolo[2,3-b]pyridin-1(6H)-yl)-1H-pyrazol-1-yl) acetonitrile (0.1 g, 46%). ¹HNMR (300 MHz, DMSO-d₆); δ=11.93 (s, 1H), 8.91 (s, 1H), 8.05 (s, 1H), 7.91 (s, 1H), 7.56 (s, 1H), 7.02 (d, J=7.5 Hz, 1H), 6.89 (s, 1H), 6.59 (d, J=7.5 Hz, 1H), 4.85 (s, 2H), 4.39 (s, 2H) 3.65 (s, 1H) ppm; ¹³CNMR (75 MHz, DMSO-d₆) δ 153.88, 151.17, 149.67, 142.18, 141.33, 130.75, 129.75, 129.07, 127.19, 120.77, 116.36, 115.63, 107.96.104.07, 100.58, 98.67, 57.38, 46.17 ppm; HRMS (ESI): m/z [M+H]⁺. C₁₈H₁₄N₇O₂

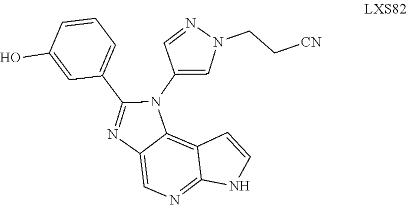 [SMILES: N#CCCn1cc(-n2c(-c3cccc(O)c3)nc3cnc4[nH]ccc4c32)cn1]

### 3-(4-(2-(3-Hydroxyphenyl)imidazo[4,5-d]pyrrolo[2,3-b]pyridin-1(6H)-yl)-1H-pyrazol-1-yl)propanenitrile

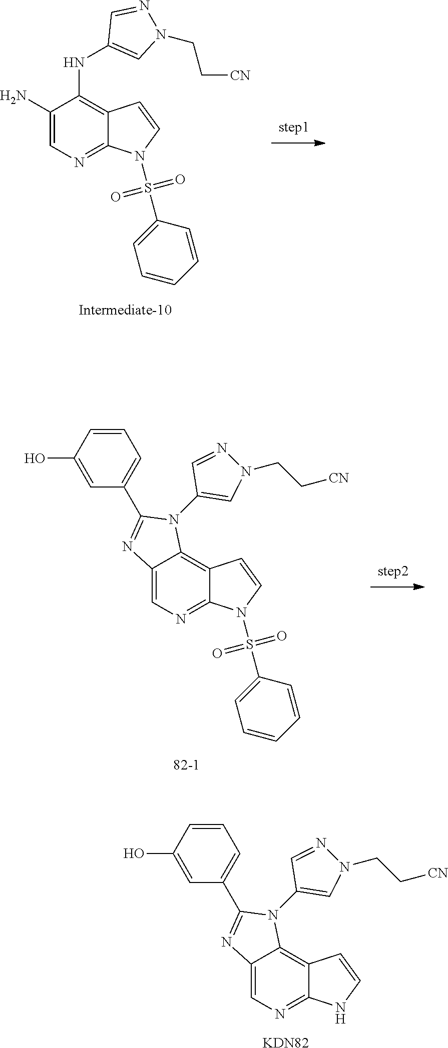

### Step 1: Dissolve 3-(4-((5-amino-1-(phenylsulfonyl)-1H-pyrrolo[2,3-b]pyridin-4-yl)amino)-1H-pyrazol-1-yl)propanenitrile (0.3 g, 0.7 mmol) in 10 mL of DMF, add Na₂S₂O₅ (0.7 g, 3.5 mmol) followed by adding 3-hydroxybenzaldehyde (0.2 g, 1.0 mmol). Stir for 12 h at

90° C. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline and dried over anhydrous sodium sulfate to yield 3-(4-(2-(3-hydroxyphenyl)-6-(phenylsulfonyl)imidazo[4,5-d]pyrrolo[2,3-b]pyridin-1(6H)-yl)-1H-pyrazol-1-yl)propanenitrile as a yellow solid (0.3 g, 79%). HRMS (ESI): m/z [M+H]⁺. C₂₆H₂₀N₇O₃S calculated value 510.1343, measured value 510.1351.

Step 2: Dissolve 3-(4-(2-(3-hydroxyphenyl)-6-(phenylsulfonyl)imidazo[4,5-d]pyrrolo[2,3-b]pyridin-1(6H)-yl)-1H-pyrazol-1-yl)propanenitrile (0.3 g, 0.6 mmol) in a mixture of 5 mL of tetrahydrofuran and 5 mL of methanol, add 5 mL of 1 M sodium hydroxide and then stir at room temperature for 5 hours. The reaction was monitored completion by TLC for 5 hours. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS82: 3-(4-(2-(3-Hydroxyphenyl)imidazo[4,5-d]pyrrolo[2,3-b]pyridin-1(6H)-yl)-1H-pyrazol-1-yl)propanenitrile (0.1 g, 46%). ¹HNMR (300 MHz, DMSO-d₆): δ=11.79 (s, 1H), 8.83 (s, 1H), 8.06 (s, 1H), 7.94 (s, 1H), 7.84 (d, J=9.0 Hz, 1H), 7.59 (s, 1H), 7.34 (t, J=9.0 Hz, 1H), 7.04 (s, 1H), 6.91 (d, J=9.0 Hz, 1H), 6.89 (s, 1H), 5.35 (s, 1H), 5.03 (t, J=7.5 Hz, 2H), 3.29 (t, J=7.5 Hz, 2H) ppm; ¹³CNMR (75 MHz, DMSO-d₆) δ 157.59, 149.56, 143.43, 142.19, 132.08, 130.75, 130.61, 129.77, 120.06, 127.18, 120.74, 120.15, 117.74, 115.97, 115.61, 112.95, 100.59, 98.65, 50.65, 18.66 ppm; HRMS (ESI): m/z [M+H]⁺. C₂₀H₁₆N₇O calculated value 370.1411, measured value 370.1419.

### Example 83

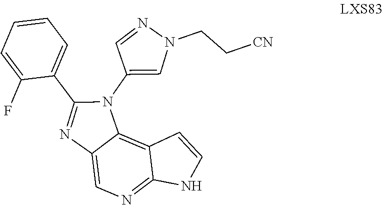 [SMILES: N#CCCn1cc(-n2c(-c3ccccc3F)nc3cnc4[nH]ccc4c32)cn1]

### 3-(4-(2-(2-Fluorophenyl)imidazo[4,5-d]pyrrolo[2,3-b]pyridin-1(6H)-yl)-1H-pyrazol-1-yl)propanenitrile

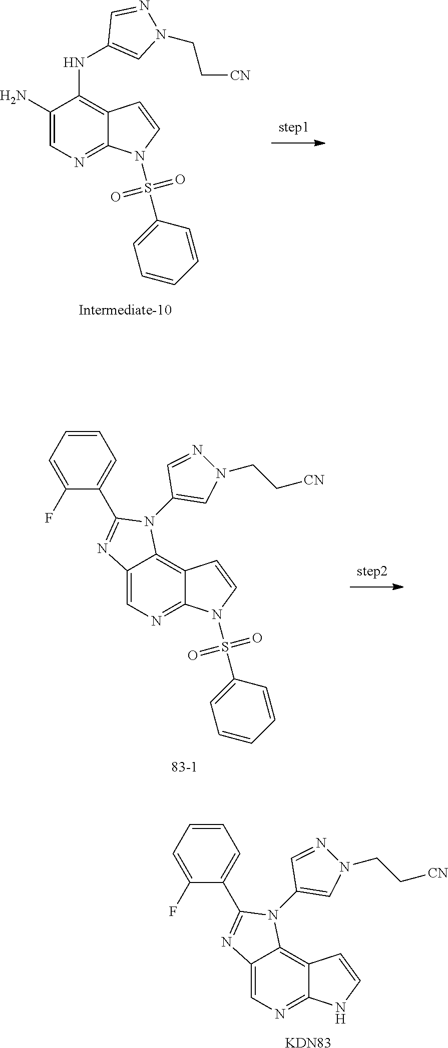

Step 1: Dissolve 3-(4-((5-amino-1-(phenylsulfonyl)-1H-pyrrolo[2,3-b]pyridin-4-yl)amino)-1H-pyrazol-1-yl)propanenitrile (0.3 g, 0.7 mmol) in 10 mL of DMF, add Na₂S₂O₅ (0.7 g, 3.5 mmol) followed by adding 2-fluorobenzaldehyde (0.2 g, 1.0 mmol). Stir for 12 h at 90 C. The reaction was monitored for completion by TLC. Saturated sodium bicarbonate was added until the reaction solution was weakly basic, and the organic phase was separated. The aqueous phase was extracted twice using dichloromethane, be organic phases were combined and washed with saturated saline and dried over anhydrous sodium sulfate to yield 3-(4-(2-(2-fluorophenyl)-6-(phenylsulfanyl)imidazo[4,5-d]pyrrolo[2,3-b]pyridin-1(6H)-yl)-1H-pyrazol-1-yl)propanenitrile as a yellow solid (0.3 g, 80%). HRMS (EST): m/z [M+H]⁺. C₂₆H¹⁹FN₇O₂S calculated value 512.1299, measured value 512.1301.

Step 2: Dissolve 3-(4-(2-(2-fluorophenyl)-6-(phenylsulfonyl)imidazo[4,5-d]pyrrolo[2,3-b]pyridin-1(6H)-yl)-1H-pyrazol-1-yl)propanenitrile (0.3 g, 0.6 mmol) in a mixture of 5 mL of tetrahydrofuran and 5 mL of methanol, add 5 mL of 1 M sodium hydroxide and then stir at room temperature for 5 hours. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate=1:1) to yield LXS83: 3-(4-(2-(2-Fluorophenyl)imidazo[4,5-d]pyrrolo[2,3-b]pyridin-1(6H)-yl)-1H-pyrazol-1-yl)propanenitrile (0.1 g, 46%), 46%). ¹HNMR (30) MHz, DMSO-d₆): δ=12.07 (s, 1H), 8.78 (s, 1H), 8.05 (s, 1H), 7.96 (s, 1H), 7.71-7.77 (m, 2H), 7.56 (s, 1H), 7.49 (d, J=7.5 Hz, 1H), 7.28 (t, J=7.5 Hz, 1H), 6.88 (s, 1H), 5.04 (t, J=9.0 Hz, 2H), 3.21 (t, J=9.0 Hz, 2H) ppm; ¹³CNMR (75 MHz, DMSO-d₆) δ 158.39, 148.63, 143.39, 142.17, 130.77, 130.38, 129.76, 129.16, 129.06, 127.17, 124.88, 123.59, 120.74, 117.74, 115.69, 114.74, 101.76, 100.43, 51.98, 22.51 ppm; HRMS (ESI): m/z [M+H]⁺. C₂₀H₁₆FN₇ 372.1367, measured value 372.1377.

### Example 84

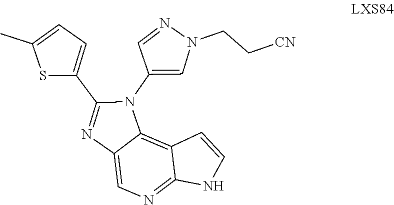 [SMILES: Cc1ccc(-c2nc3cnc4[nH]ccc4c3n2-c2cnn(CCC#N)c2)s1]

### 3-(4-(2-(5-Methylthiophen-2-yl)imidazo[4,5-d]pyrrolo[2,3-b]pyridin-1(6H)-yl)-1H-pyrazol-1-yl)propanenitrile

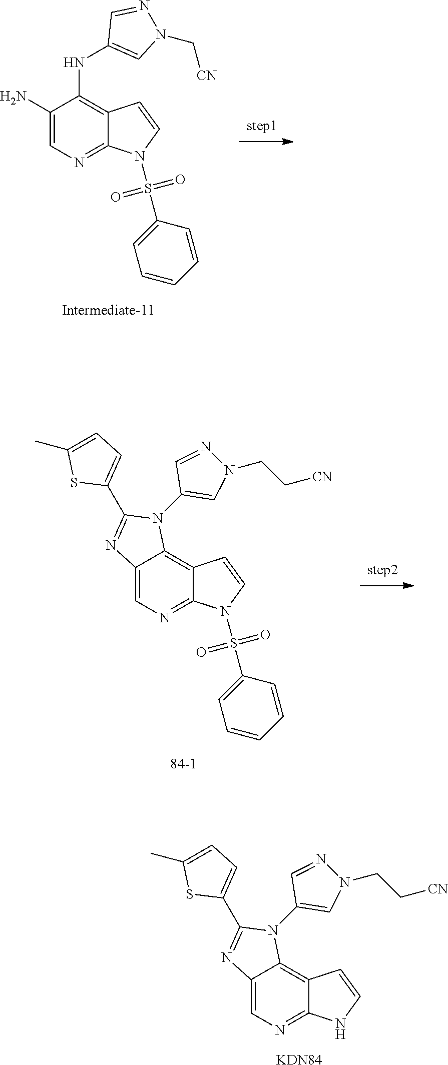

Step 1: Dissolve 3-(4-((5-amino-1-(phenylsulfonyl)-1H-pyrrolo[2,3-b]pyridin-4-yl)amino)-1H-pyrazol-1-yl)propanenitrile (0.3 g, 0.7 mmol) in 10 mL of DMF, add Na₂S₂O₅ (0.7 g, 3.5 mmol) followed by adding 5-methyl-2-thiophenecarboxaldehyde (0.2 g, 1.0 mmol). Stir for 12 h at 90° C. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline and dried over anhydrous sodium sulfate to yield 3-(4-(2-(5-methylthiophen-2-yl)-6-(phenylsulfonyl)imidazo[4,5-d]pyrrolo[2,3-b]pyridin-1(6H)-yl)-1H-pyrazol-1-yl)propanenitrile as a yellow solid (0.3 g, 80%). HRMS (ESI); m/z [M+H]⁺. C₂₅H₂₀N₇O₂S₂ calculated value 514.1114, measured value 514.1119.

Step 2: Dissolve 3-(4-(2-(5-methylthiophen-2-yl)-6-(phenylsulfonyl)imidazo[4,5-d]pyrrolo[2,3-b]pyridin-1(6H)-yl)-1H-pyrazol-1-yl)propanenitrile (0.3 g, 0.6 mmol) in a solvent mixture of 5 mL tetrahydrofuran and 5 mL methanol. After the addition of 5 mL of 1M sodium hydroxide, the mixture was then stirred at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate=1:1) to yield LXS84: 3-(4-(2-(5-methylthiophen-2-yl)imidazo[4,5-d]pyrrolo[2,3-b]pyridin-1(6H)-yl)-1H-pyrazol-1-yl)propanenitrile (0.1 g, 46%). ¹HNMR (300 MHz, DMSO-d₆): δ=11.93 (s, 1H), 8.83 (s, 1H), 8.06 (s, 1H), 7.99 (s, 1H), 7.56 (s, 1H), 7.51 (d, J=7.5 Hz, 1H), 6.89 (s, 1H), 6.83 (d, J=7.5 Hz, 1H), 5.10 (t, J=6.0 Hz, 2H), 3.26 (t, J=6.0 Hz, 2H), 2.36 (s, 3H) ppm; ¹³CNMR (75 MHz, DMSO-d₆) δ 151.63, 142.17, 141.63, 141.30, 134.38, 130.74, 129.74.129.06, 127.59, 127.50, 127.14, 120.79, 117.78, 115.62, 101.86, 99.38, 51.96, 15.93, 15.22 ppm: HRMS (ESI): m/z [M+H]⁺. C₁₉H₁₆N₇S calculated value 374.1182, measured value 374.1189.

### Example 85

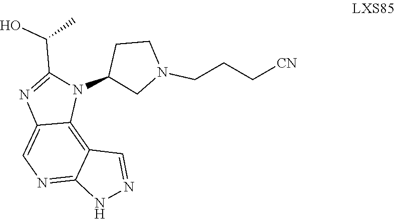 [SMILES: C[C@@H](O)c1nc2cnc3[nH]ncc3c2n1[C@H]1CCN(CCCC#N)C1]

### 4-((S)-3-(2-((R)-1-Hydroxyethyl)imidazo[4,5-d]pyrazolo[3,4-b]pyridin-1(6H)-yl)pyrrolidin-1-yl)butanenitrile

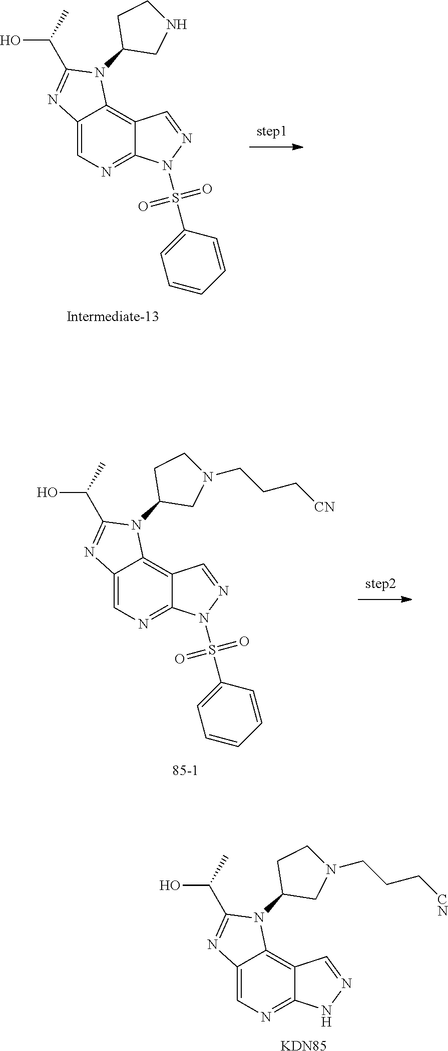

Step 1: Dissolve (R)-1-(6-(phenylsulfonyl)-1-((S)-pyrrolidin-3-yl)-1,6-dihydroimidazo[4,5-d]pyrazolo[3,4-b]pyridin-2-yl) ethanol (0.3 g, 0.7 mmol) in 10 mL of tetrahydrofuran, add DIPEA (0.2 g, 1.4 mmol) followed by the slow dropwise addition of 4-bromobutyronitrile (0.2 g, 1.1 mmol). Raise the temperature to maintain reflux and stir for 3 h. Monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield 4-((S)-3-(2-((R)-1-hydroxyethyl)-6-(phenylsulfonyl)imidazo[4,5-d]pyrazolo[3,4-b]pyridin-1(6H)-yl)pyrrolidin-1-yl)butyronitrile as a pale yellow oil (0.2 g, 57%). The product can be used directly in the next step without further purification. HRMS (ESI): m/z [M+H]⁺. C₂₃H₂₆N₇O₃S calculated value 480.1812, measured value 480.1825.

Step 2: 4-((S)-3-(2-((R)-1-Hydroxyethyl)-6-(phenylsulfonyl)imidazo[4,5-d]pyrazolo[3,4-b]pyridin-1(6H)-yl)pyrrolidin-1-yl)butyronitrile (0.2 g, 0.4 mmol) was dissolved in a solvent mixture of 5 mL tetrahydrofuran and 5 mL methanol. After the addition of 5 mL of 1 M sodium hydroxide, stir the mixture for 5 h at room temperature and monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate=1:1) to yield LXS85: 4-((S)-3-(2-((R)-1-hydroxyethyl)imidazo[4,5-d]pyrazolo[3,4-b]pyridin-1(6H)-yl)pyrrolidin-1-yl)butanenitrile (0.1 g, 71%). ¹HNMR (300 MHz, DMSO-d₆): δ=12.54 (s, 1H), 8.89 (s, 1H), 7.55 (s, 1H), 4.65-4.70 (m, 1H), 3.64-3.81 (m, 1H), 2.56-2.83 (m, 2H), 2.43 (t, J=9.0 Hz, 2H), 2.23-2.31 (m, 2H), 1.90-2.15 (m, 2H), 1.87 (t, J=9.0 Hz, 2H), 1.66-1.76 (m, 2H), 1.41 (d, J=6.0 Hz, 3H) ppm; ¹³CNMR (75 MHz, DMSO-d₆) δ 154.79.148.53, 140.85, 140.53, 132.46, 125.49, 119.39, 104.75, 63.68, 58.52, 58.41, 55.92, 55.51, 27.61, 22.85, 15.94, 15.11 ppm; HRMS (ESI): m/z [M+H]⁺. C₁₇H₂₂N₇O calculated values 340.1880, measured value 340.1888.

### Example 86

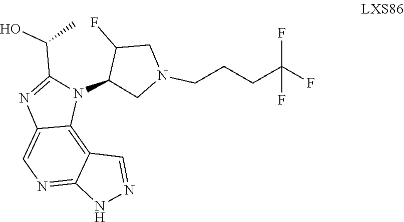 [SMILES: C[C@@H](O)c1nc2cnc3[nH]ncc3c2n1[C@@H]1CN(CCCC(F)(F)F)CC1F]

### (1R)-1-(1-((3R)-4-fluoro-1-(4,4,4-trifluorobutyl)pyrrolidin-3-yl)-1,6-dihydroimidazo[4,5-d]pyrrolo[2,3-b]pyridin-2-yl)ethan-1-ol

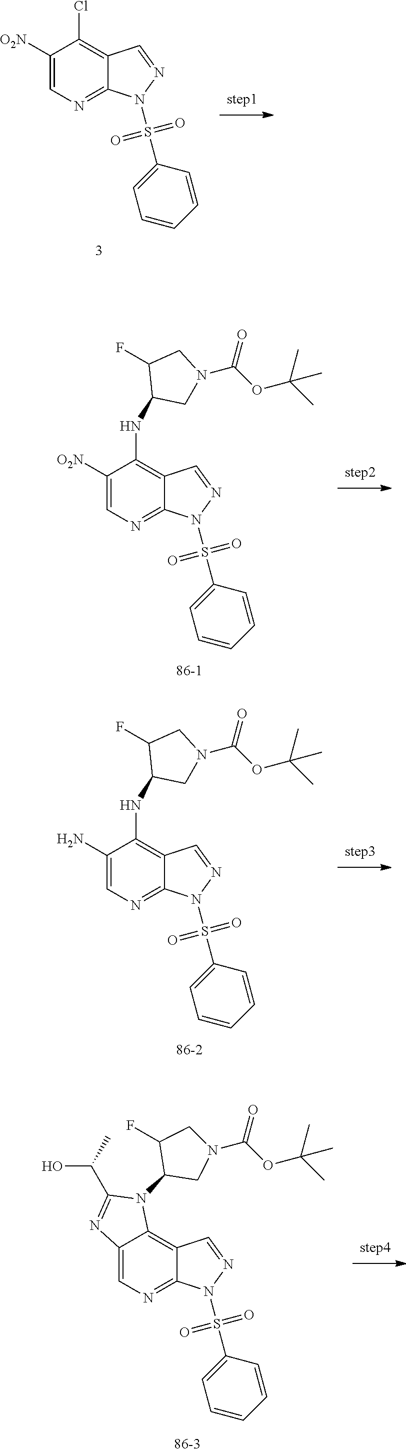 [SMILES: O=[N+]([O-])c1cnc2c(cnn2S(=O)(=O)c2ccccc2)c1Cl ; CC(C)(C)OC(=O)N1CC(F)[C@H](Nc2c([N+](=O)[O-])cnc3c2cnn3S(=O)(=O)c2ccccc2)C1 ; CC(C)(C)OC(=O)N1CC(F)[C@H](Nc2c(N)cnc3c2cnn3S(=O)(=O)c2ccccc2)C1 ; C[C@@H](O)c1nc2cnc3c(cnn3S(=O)(=O)c3ccccc3)c2n1[C@@H]1CN(C(=O)OC(C)(C)C)CC1F]

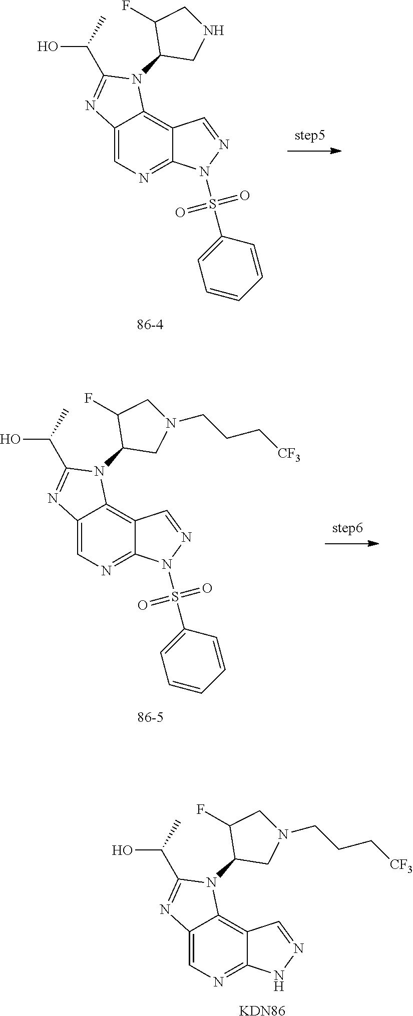

Step 1: 4-Chloro-5-nitro-1-(phenylsulfonyl)-1H-pyrrolo[2,3-b]pyridine (1.0 g, 3.0 mmol) was dissolved in 10 mL of tetrahydrofuran. Then, DIPEA (0.8 g, 6.0 mmol) and (3R) tert-butyl 3-amino-4-fluoropyrrolidine-1-carboxylate (0.9 g, 4.5 mmol) were added and stirred for 4h while maintaining reflux at an elevated temperature. The reaction was monitored for completion by TLC. Concentrate under vacuum to obtain a yellow oily liquid. Triturate and cure with an appropriate amount of methanol to yield (4R)-tert-butyl 3-fluoro-4-((5-nitro-1-(phenylsulfonyl)-1H-pyrrolo[2,3-b]pyridin-4-yl)amino)pyrrolidine-1-carboxylate as a yellow solid (1.2 g, 80%). The product can be used directly in the next step without further purification. HRMS (ESI): m/z [M+H]⁺. C₂₂H₂₅FN₅O₆S calculated value 506.1504, measured value 506.1509.

Step 2: Dissolve (4R)-tert-butyl 3-fluoro-4-((5-nitro-1-(phenylsulfonyl)-1H-pyrrolo[2,3-b]pyridin-4-yl)amino)pyrrolidine-1-carboxylate (1.2 g, 2.4 mmol) in 20 mL of methanol, add palladium carbon (0.1 g, 10%) and then use hydrogen to replace the air in the reaction flask more than three times. The reaction was carried out in hydrogen atmosphere and stirred at room temperature for 12 h. The reaction was monitored for completion by TLC. After filtration, the filtrate was collected and concentrated under vacuum to yield (4R)-tert-butyl 3-fluoro-4-((5-amino-1-(phenylsulfonyl)-1H-pyrrolo[2,3-b]pyridin-4-yl)amino)pyrrolidine-1-carboxylate as a light pink foamy solid (1.1 g, 97%). The product can be used directly in the next step without further purification. HRMS (EST): m/z [M+H]⁺. C₂₂H₂₇FN₅O₄S calculated value 476.1762, measured value 476.1770.

Step 3: Dissolve triethyloxonium tetrafluoroborate (1.2 g, 6.3 mmol) and (R)-lactamideide (0.6 g, 6.3 mmol) in 10 mL of tetrahydrofuran, stir for 3 h at room temperature and then concentrate the mixture under vacuum to obtain an oily liquid, followed by the addition of 10 mL of ethanol to dissolve. Then, add (4R)-tert-butyl 3-fluoro-4-((5-amino-1-(phenylsulfonyl)-1H-pyrrolo[2,3-b]pyridin-4-yl)amino)pyrrolidine-1-formate (1.0 g, 2.1 mmol). Raise the temperature to maintain reflux and stir for 3 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum. The residue was purified using a silica gel column (petroleum ether:ethyl acetate=1:1) to yield (4R)-tert-butyl 3-fluoro-4-(2-((R)-1-hydroxyethyl)-6-(phenylsulfonyl)imidazo[4,5-d]pyrrolo[2,3-b]pyridin-1(6H)-yl)pyrrolidine-1-carboxylate as a green oil (0.5 g, 45%). HRMS (EST): m/z [M+H]⁺. C₂₅H₂₉FN₅O₅S calculated value 530.1868, measured value 530.1870.

Step 4: Dissolve (4R)-tert-butyl 3-fluoro-4-(2-((R)-1-hydroxyethyl)-6-(phenylsulfonyl)imidazo[4,5-d]pyrrolo[2,3-b]pyridin-1 (6H)-yl)pyrrolidine-1-carboxylate (0.5 g, 0.9 mmol) in 10 mL of dichloromethane, slowly add trifluoroacetic acid (1.0 g, 9.0 mmol) and stir at room temperature for 12 h. Concentrate under vacuum to yield (1R)-1-(1-(((3R)-4-fluoropyrrolidin-3-yl)-6-(phenylsulfonyl)-1,6-dihydroimidazo[4,5-d]pyrrolo[2,3-b]pyridin-2-yl)ethanol as a light brown oil (0.4 g, 99%). The product can be used directly in the next step without further purification. HRMS (ESI): m/z [M+H]⁺. C₂₀H₂₁FN₅O₃S calculated value 430.1344, measured value 430.1350.

Step 5: Dissolve (1R)-1-(1-(((3R)-4-fluoropyrrolidin-3-yl)-6-(phenylsulfonyl)-1,6-dihydroimidazo[4,5-d]pyrrolo[2,3-b]pyridin-2-yl)ethanol (0.3 g, 0.7 mmol) in 10 mL of tetrahydrofuran, add DIPEA (0.2 g, 1.4 mmol) and then add 4,4,4-trifluoro-1-iodobutane (0.3 g, 1.1 mmol) dropwise. Raise the temperature to maintain reflux and stir for 3 h. Monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield (1R)-1-(1-(((3R)-4-fluoro-1-(4,4,4-trifluorobutyl)pyrrolidin-3-yl)-6-(phenylsulfonyl)-1,6-dihydroimidazo[4,5-d]pyrrolo[2,3-b]pyridin-2-yl) ethanol as a pale yellow oil (0.2 g, 53%), 53%). The product can be used directly in the next step without further purification. HRMS (ESI): m/z [M+H]⁺. C₂₄H₂₆F₄N₅O₃S calculated value 540.1687, measured value 540.1689.

Step 6: (1R)-1-(1-(((3R)-4-fluoro-1-(4,4,4-trifluorobutyl)pyrrolidin-3-yl)-6-(phenylsulfonyl)-1,6-dihydroimidazo[4,5-d]pyrrolo[2,3-b]pyridin-2-yl)ethanol (0.2 g, 0.4 mmol) was dissolved in a solvent mixture of 5 mL tetrahydrofuran and 5 mL methanol. After adding 5 mL of 1 M sodium hydroxide, the reaction was stirred at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate =1:1) to yield LXS86: (1R)-1-(1-((3R)-4-fluoro-1-(4,4,4-trifluorobutyl)pyrrolidin-3-yl)-1,6-dihydroimidazo[4,5-d]pyrrolo[2,3-b]pyridin-2-yl)ethan-1-ol (0.1 g, 68%). ¹HNMR (300 MHz, DMSO-d₆): δ=11.94 (s, 1H), 8.68 (s, 1H), 7.54 (s, 1H), 6.77 (s, 1H), 4.684.72 (m, 1H), 3.91-4.06 (m, 1H), 3.30-3.41 (m, 1H), 2.56-2.81 (m, 2H), 2.54 (t, J=4.5 Hz, 2H), 2.29-2.51 (m, 2H), 1.64-1.81 (m, 2H), 1.46 (d, J=9.0 Hz, 3H), 1.36-1.42 (m, 2H) ppm; ¹³ C NMR (75 MHz, DMSO-d₆) δ 151.89, 148.53, 142.19, 129.06, 127.18, 126.84, 120.77, 115.69.98.56, 91.20, 63.91, 61.77, 56.33, 54.88, 51.67, 37.71, 22.86, 11.67 ppm, HRMS (ESI): m/z [M+H]⁺. C₁₈H₂₂F₄N₅₀ calculated value 400.1755, measured value 400.1770.

### Example 87

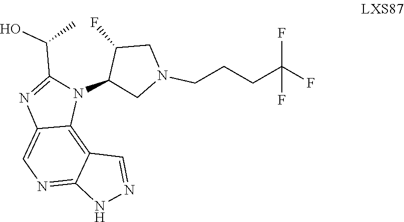 [SMILES: C[C@@H](O)c1nc2cnc3[nH]ncc3c2n1[C@@H]1CN(CCCC(F)(F)F)C[C@H]1F]

(R)-1-(1-((3R,4R)-4-fluoro-1-(4,4,4-trifluorobutyl)pyrrolidin-3-yl)-1,6-dihydroimidazo[4,5-d]pyrrolo[2,3-b]pyridin-2-yl)ethan-1-olUsing (3R,4R)-tert-butyl 3-amino-4-fluoropyrrolidine-1-carboxylate instead of (3R)-tert-butyl 3-amino-4-fluoropyrrolidine-1-carboxylate, LXS 87 was prepared in a similar preparation way described in Example 86. The crude product was purified using a silica gel column (petroleum ether:ethyl acetate=1:1) to yield LXS87: (R)-1-(1-((3R,4R)-4-fluoro-1-(4,4,4-trifluorobutyl)pyrrolidin-3-yl)-1,6-dihydroimidazo[4,5-d]pyrrolo[2,3-b]pyridin-2-yl)ethan-1-olpyridin-2-yl) ethanol (0.1 g, 60%). ¹HNMR (300 MHz, DMSO-d₆): δ=11.99 (s, 1H), 8.78 (s, 1H), 7.58 (s, 1H), 6.75 (s, 1H), 4.64-4.71 (m, 1H), 3.90-4.05 (m, 1H), 3.31-3.55 (m, 1H), 2.53-2.87 (m, 2H), 2.50 (t, J=6.0 Hz, 2H), 2.23-2.47 (m, 2H), 1.73-1.88 (m, 2H), 1.41 (d, J=6.0 Hz, 3H), 1.33-1.44 (m, 2H) ppm; ¹³ CNMR (75 MHz, DMSO-d₆) δ 148.97, 148.53, 142.19, 129.00, 127.65, 126.88, 120.73, 115.69, 97.63, 91.28, 63.63, 61.79, 56.39, 54.85, 51.63, 37.77, 22.81, 11.65 ppm: HRMS (ESI): m/z [M+H]⁺. C₁₈H₂₂F₄N₅O calculated value 400.1755, measured value 400.1768.

### Example 88

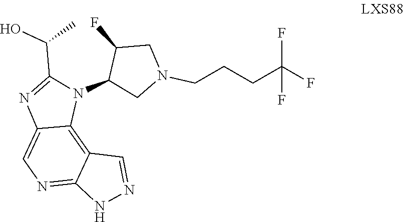 [SMILES: C[C@@H](O)c1nc2cnc3[nH]ncc3c2n1[C@@H]1CN(CCCC(F)(F)F)C[C@@H]1F]

### (R)-1-(1-((3R,4S)-4-fluoro-1-(4,4,4-trifluorobutyl)pyrrolidin-3-yl)-1,6-dihydroimidazo[4,5-d]pyrrolo[2,3-b]pyridin-2-yl)ethan-1-ol

Using (3R,4R)-tert-butyl 3-amino-4-fluoropyrrolidine-1-carboxylate instead of (3R)-tert-butyl 3-amino-4-fluoropyrrolidine-1-carboxylate, LXS 88 was prepared in a similar preparation way described in Example 86. The crude product was purified using a silica gel column (petroleum ether:ethyl acetate=1:1) to yield LXS88: (R)-1-(1-((3R,4S)-4-fluoro-1-(4,4,4-trifluorobutyl)pyrrolidin-3-yl)-1,6-dihydroimidazo[4,5-d]pyrrolo[2,3-b]pyridin-2-yl)ethan-1-ol (0.1 g, 50%). ¹HNMR (300 MHz, DMSO-d₆): δ=12.13 (s, 1H), 8.81 (s, 1H), 7.60 (s, 1H), 6.57 (s, 1H), 4.58-4.70 (m, 1H), 3.90-4.05 (m, 1H), 3.33-3.42 (m, 1H), 2.51-2.86 (m, 2H), 2.42 (t, J=9.0 Hz, 2H), 2.30-2.41 (m, 2H), 1.66-1.82 (m, 2H), 1.49 (d, J=9.0 Hz, 3H), 1.39-1.46 (m, 2H) ppm; ¹³CNMR (75 MHz, DMSO-d₆) δ 151.45, 158.55, 142.19, 129.05, 127.16, 126.86, 120.74, 115.64, 98.56, 91.55, 63.65, 61.75, 56.38, 54.85, 51.68, 37.77, 22.89, 11.56 ppm; HRMS (ESI): m/z [M+H]⁺. C₁₈H₂₂F₄N₅₀ calculated value 400.1755, measured value 400.1763.

### Example 89

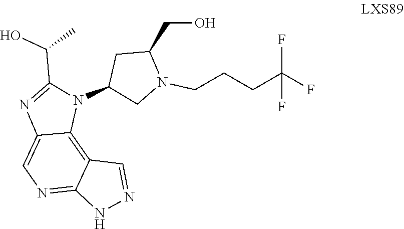 [SMILES: C[C@@H](O)c1nc2cnc3[nH]ncc3c2n1[C@H]1C[C@@H](CO)N(CCCC(F)(F)F)C1]

(R)-1-(1-((3S,5S)-5-(Hydroxymethyl)-1-(4,4,4-trifluorobutyl)pyrrolidin-3-yl)-1,6-dihydroimidazo[4,5-d]pyrrolo[2,3-b]pyridin-2-yl)ethan-1-olUsing tert-butyl (2S,4S)-4-amino-2-(hydroxymethyl)pyrrolidine-1-carboxylate instead of tert-butyl (3R)-3-amino-4-fluoropyrrolidine-1-carboxylate, LXS 89 was prepared in a similar preparation way described in Example 86. The crude product was purified using a silica gel column (petroleum ether:ethyl acetate=1:1) to yield LXS89: (R)-1-(1-((3S,5S)-5-(hydroxymethyl)-1-(4,4,4-trifluorobutyl)pyrrolidin-3-yl)-1,6-dihydroimidazo[4,5-d]pyrrolo[2,3-b]pyridin-2-yl)ethan-1-ol (0.1 g, 51%). ¹HNMR (300 MHz, DMSO-d₆): δ=11.98 (s, 1H), 8.78 (s, 1H), 7.59 (s, 1H), 6.88 (s, 1H), 4.68-4.74 (m, 1H), 3.79-3.85 (m, 1H), 3.34 (d, J=6.0 Hz, 2H), 2.59-2.82 (m, 2H), 2.43 (t, J=4.5 Hz, 2H), 2.33-2.41 (m, 1H), 1.87-2.12 (m, 2H), 1.64-1.81 (m, 2H), 1.47 (d, J=6.0 Hz, 3H), 1.36-1.40 (m, 2H) ppm; ¹³CNMR (75 MHz, DMSO-d₆) δ 148.94, 148.57, 142.19, 129.04, 127.11, 126.88, 120.75, 115.63, 98.67, 68.74, 63.67, 62.55, 56.71, 56.21, 54.18, 37.79, 28.31, 22.84, 10.49 ppm; HRMS (ESI): m/z [M+H]⁺. C₁₉H₂₅F₃N₅O₂ calculated value 412.1955, measured value 412.1961.

### Example 90

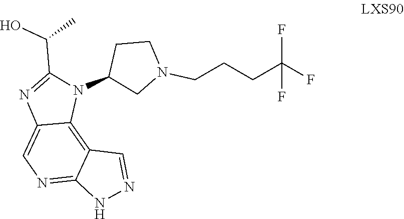 [SMILES: C[C@@H](O)c1nc2cnc3[nH]ncc3c2n1[C@H]1CCN(CCCC(F)(F)F)C1]

### (R)-1-(1-((S)-1-(4,4,4-trifluorobutyl)pyrrolidin-3-yl)-1,6-dihydroimidazo[4,5-d]pyrazolo[3,4-b]pyridin-2-yl)ethan-1-ol

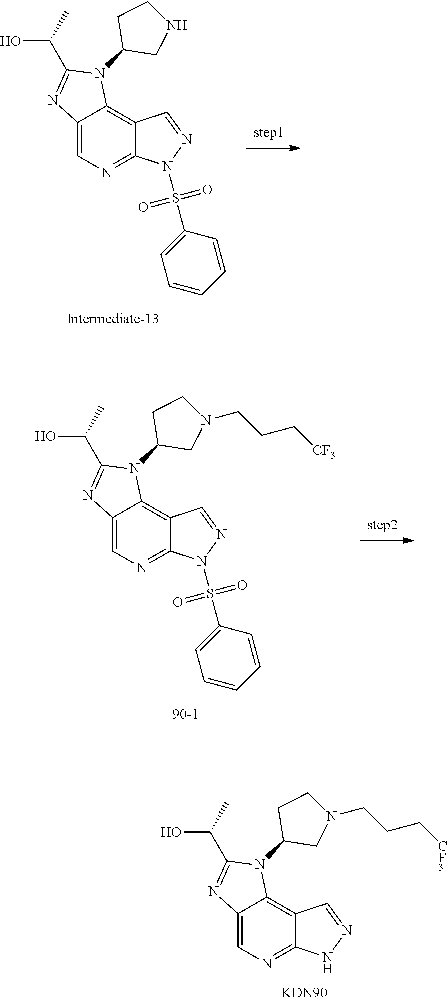

Step 1: Dissolve (R)-1-(6-(phenylsulfonyl)-1-((S)-pyrrolidin-3-yl)-1,6-dihydroimidazo[4,5-d]pyrazolo[3,4-b]pyridin-2-yl)ethanol (0.3 g, 0.7 mmol) in 10 mL of tetrahydrofuran, add DIPEA (0.2 g, 1.4 mmol) followed by the slow dropwise addition of 4,4,4-trifluoro-1-iodobutane (0.3 g, 1.1 mmol). Raise the temperature to maintain reflux and stir for 3 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield (R)-1-(6-(phenylsulfonyl)-1-((S)-1-(4,4,4-trifluorobutyl)pyrrolidin-3-yl)-1,6-dihydroimidazo[4,5-d]pyrazolo[3,4-b]pyridin-2-yl) ethanol as a pale yellow oil (0.2 g, 52%). The product can be used directly in the next step without further purification. HRMS (ESI): m/z [M+H]⁺. C₂₃H₂₆F₃N₆O₃S calculated value 523.1734, measured value 523.1740.

Step 2: (R)-1-(6-(Phenylsulfonyl)-1-((S)-1-(4,4,4-trifluorobutyl)pyrrolidin-3-yl)-1,6-dihydroimidazo[4,5-d] pyrazolo[3,4-b]pyridin-2-yl) ethanol (0.2 g, 0.4 mmol) was dissolved in a solvent mixture of 5 mL tetrahydrofuran and 5 mL methanol, and add 5 mL of IM sodium hydroxide and then stir at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS90: (R)-1-(1-((S)-1-(4,4,4-trifluorobutyl)pyrrolidin-3-yl)-1,6-dihydroimidazo[4,5-d]pyrazolo[3,4-b]pyridin-2-yl)ethan-1-ol (0.1 g, 68%). ¹HNMR (300 MHz, DMSO-d₆): δ=13.54 (s, 1H), 8.82 (s, 1H), 7.58 (s, 1H), 4.65-4.73 (m, 1H), 3.64-3.79 (m, 2H), 2.53-2.81 (m, 2H), 2.41 (t, J=6.0 Hz, 2H), 2.20-2.33 (m, 2H), 1.91-2.14 (m, 2H), 1.85 (m, 2H), 1.41 (d, J=9.0 Hz, 3H), 1.27-1.37 (m, 2H) ppm; ¹³CNMR (75 MHz, DMSO-d₆) δ 154.75, 148.52, 140.88, 140.51, 132.48, 126.84, 125.47, 105.37, 63.68, 58.51, 58.43, 56.38, 55.91, 38.88, 27.91, 22.86, 9.56 ppm; HRMS (ESI): m/z [M+H]⁺. C₁₇H₂₂F₃N₆₀ calculated value 383.1802, measured value 383.1800.

### Example 91

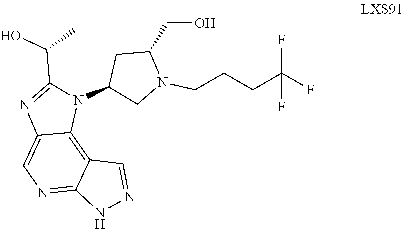 [SMILES: C[C@@H](O)c1nc2cnc3[nH]ncc3c2n1[C@H]1C[C@H](CO)N(CCCC(F)(F)F)C1]

(R)-1-(1-((3S,5R)-5-(hydroxymethyl)-1-(4,4,4-trifluorobutyl)pyrrolidin-3-yl)-1,6-dihydroimidazo[4,5-d]pyrrolo[2,3-b]pyridin-2-yl)ethan-1-olLXS91 was prepared by replacing tert-butyl (3R)-3-amino-4-fluoropyrrolidine-1-carboxylate with tert-butyl (2R,4S)-4-amino-2-(hydroxymethyl)pyrrolidine-1-carboxylate, using a preparation method similar to Example 86, and the crude product was purified using a silica gel column (petroleum ether:ethyl acetate=1:1) to yield LXS91: (R)-1-(1-((3S,5R)-5-(hydroxymethyl)-1-(4,4,4-trifluorobutyl)pyrrolidin-3-yl)-1,6-dihydroimidazo[4,5-d]pyrrolo[2,3-b]pyridin-2-yl)ethan-1-ol (0.1 g, 43%). ¹ HNMR (300 MHz, DMSO-d₆): δ=11.68 (s, 1H), 8.73 (s, 1H), 7.61 (s, 1H), 6.89 (s, 1H), 4.65-4.71 (m, 1H), 3.76-3.89 (m, 1H), 3.35 (d, J=6.0 Hz, 2H), 2.64-2.81 (m, 2H), 2.45 (t, J=7.5 Hz, 2H), 2.37-2.39 (m, 1H), 1.89-2.15 (m, 2H), 1.60-1.84 (m, 2H), 1.43 (d, J=9.0 Hz, 3H), 1.33-1.41 (m, 2H) ppm; ¹³ C NMR (75 MHz, DMSO-d₆) δ 151.87, 148.54, 142.19, 129.05, 127.18, 126.84, 120.77, 115.69, 98.56, 68.74, 63.69, 62.57, 56.71, 56.38, 54.18, 38.96, 28.54, 22.85, 13.74 ppm; HRMS (EST): m/z [M+H]⁺. C₁₉H₂₅F₃N₅O₂ calculated value 412.1955, measured value 412.1963.

### Example 92

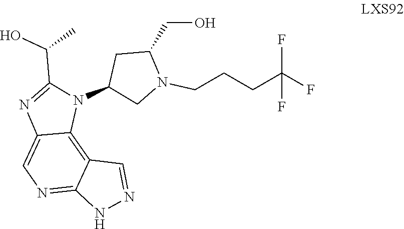 [SMILES: C[C@@H](O)c1nc2cnc3[nH]ncc3c2n1[C@H]1C[C@H](CO)N(CCCC(F)(F)F)C1]

(R)-1-(1-((3S,5R)-5-(hydroxymethyl)-1-(4,4,4-trifluorobutyl)pyrrolidin-3-yl)-1,6-dihydroimidazo[4,5-d]pyrrolo[2,3-b]pyridin-2-yl)ethan-1-olLXS92 was prepared by replacing tert-butyl (3R)-3-amino-4-fluoropyrrolidine-1-carboxylate with tert-butyl (2R,4S)-4-amino-2-(hydroxymethyl)pyrrolidine-1-carboxylate, using a preparation method similar to Example 86, and the crude product was purified using a silica gel column (petroleum ether:ethyl acetate=1:1) to yield LXS92: (R)-1-(1-((3S,5R)-5-(hydroxymethyl)-1-(4,4,4-trifluorobutyl)pyrrolidin-3-yl)-1,6-dihydroimidazo[4,5-d]pyrrolo[2,3-b]pyridin-2-yl)ethan-1-ol (0.1 g, 33%). ¹HNMR (300 MHz, DMSO-d₆): δ=13.78 (s, 1H), 8.87 (s, 1H), 7.54 (s, 1H), 4.69-4.72 (m, 1H), 3.83-3.89 (m, 1H), 3.37 (d, J=6.0 Hz, 2H), 2.61-2.80 (m, 2H), 2.47 (t, J=9.0 Hz, 2H), 2.36-2.43 (m, 1H), 1.88-2.15 (m, 2H), 1.58-1.79 (m, 2H), 1.44 (d, J=10.5 Hz, 3H), 1.38-1.43 (m, 2H) ppm; ¹³CNMR (75 MHz, DMSO-d₆) δ 155.75, 148.56, 140.83, 140.51, 132.48, 126.84, 125.42, 104.74, 68.75, 63.61, 62.51, 56.39, 56.29, 54.17, 38.67, 28.56, 22.87, 11.65 ppm; HRMS (ESI): m/z [M+H]⁺. C₁₈H₂₄F₃N₆O₂ calculated value 413.1907, measured value 413.1914.

### Example 93

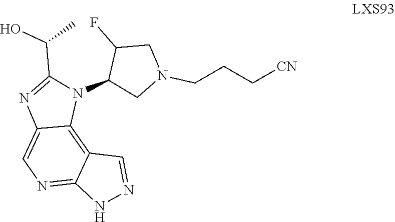 [SMILES: C[C@@H](O)c1nc2cnc3[nH]ncc3c2n1[C@@H]1CN(CCCC#N)CC1F]

### 4-((4R)-3-Fluoro-4-(2-(R)-1-hydroxyethyl)imidazo[4,5-d]pyrazolo[3,4-b]pyridin-1(6H)-yl)pyrrolidin-1-yl)butanenitrile

LXS93 was prepared in a preparation way similar to Example 86 and the crude product was purified using a silica gel column (petroleum ether:ethyl acetate=1:1) to yield LXS93: 4-((4R)-3-Fluoro-4-(2-((R)-1-hydroxyethyl)imidazo[4,5-d]pyrazolo[3,4-b]pyridin-1(6H)-yl)pyrrolidin-1-yl)butanenitrile (0.1 g, 31%). ¹HNMR (300 MHz, DMSO-d₆): δ=13.73 (s, 1H), 8.89 (s, 1H), 7.61 (s, 1H), 4.57-4.71 (m, 1H), 3.914.08 (m, 11H), 3.33-3.45 (m, 1H), 2.57-2.81 (m, 2H), 2.47 (t, J=6.0 Hz, 2H), 2.40-2.43 (m, 2H), 1.90 (t, J=9.0 Hz, 2H), 1.70-1.76 (m, 2H), 1.43 (d, J=7.5 Hz, 3H) ppm; ¹; CNMR (75 MHz, DMSO-d₆) δ 154.76, 148.54, 141.65, 140.51, 132.78, 125.47, 119.38, 104.78, 91.38, 64.89, 61.33, 55.59, 54.86, 51.69, 22.85, 17.56, 16.45 ppm: HRMS (ESI): m/z [M+H]⁺. C₁₇H₂₁FN₇O calculated value 358.1786, measured value 358.1789.

### Example 94

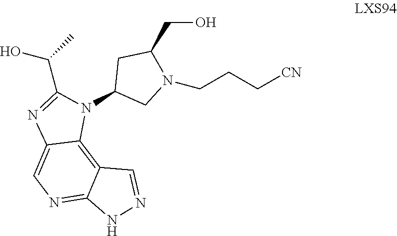 [SMILES: C[C@@H](O)c1nc2cnc3[nH]ncc3c2n1[C@H]1C[C@@H](CO)N(CCCC#N)C1]

### 4-((2S,4S)-4-(2-((R)-1-Hydroxyethyl)imidazo[4,5-d]pyrazolo[3,4-b]pyridin-1(6H)-yl)-2-(hydroxymethyl)pyrrolidin-1-yl)butanenitrile

LXS94 was prepared using a preparation method similar to Example 86 by replacing tert-butyl (3R)-3-amino-4-fluoropyrrolidine-1-carboxylate with tert-butyl (2S,4S)-4-amino-2-(hydroxymethyl)pyrrolidine-1-carboxylate, and the crude product was purified using a silica gel column (petroleum ether:ethyl acetate=1:1) to yield LXS94: 4-((2S,4S)-4-(2-((R)-1-Hydroxyethyl)imidazo[4,5-d]pyrazolo[3,4-b]pyridin-1(6H)-yl)-2-(hydroxymethyl)pyrrolidin-1-yl)butanenitrile (0.1 g, 27%). ¹HNMR (300 MHz, DMSO-d₆): δ=13.68 (s, 1H), 8.89 (s, 1H), 7.58 (s, 1H), 4.63-4.71 (m, 1H), 3.85-3.91 (m, 1H), 3.39 (d, J=9.0 Hz, 2H), 2.66-2.81 (m, 2H), 2.39 (t, J=6.0 Hz, 2H), 2.38-2.45 (m, 1H), 1.79-2.23 (m, 2H), 1.56-1.72 (m, 2H), 1.41 (d, J=10.5 Hz, 3H), 1.33-1.39 (m, 2H) ppm; ¹³CNMR (75 MHz, DMSO-d₆) δ 155.78, 149.65, 140.89, 140.32, 132.89, 124.23, 119.38, 104.73, 69.65, 63.65, 62.56, 57.76, 56.21, 53.39, 29.65, 22.89, 18.56, 14.65 ppm; HRMS (ESI): m/z [M+H]⁺. C₁₈H₂₄N₇O₂ calculated value 370.1986, measured value 370.1990.

### Example 95

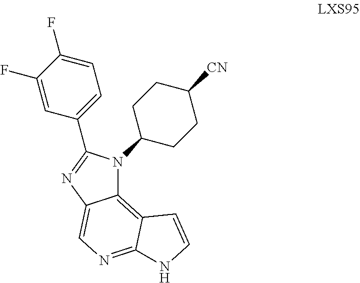 [SMILES: N#C[C@H]1CC[C@@H](n2c(-c3ccc(F)c(F)c3)nc3cnc4[nH]ccc4c32)CC1]

### trans-4-(2-(3,4-Difluorophenyl)imidazo[4,5-d]pyrrolo[2,3-b]pyridin-1(6H)-yl)cyclohexanecarbonitrile

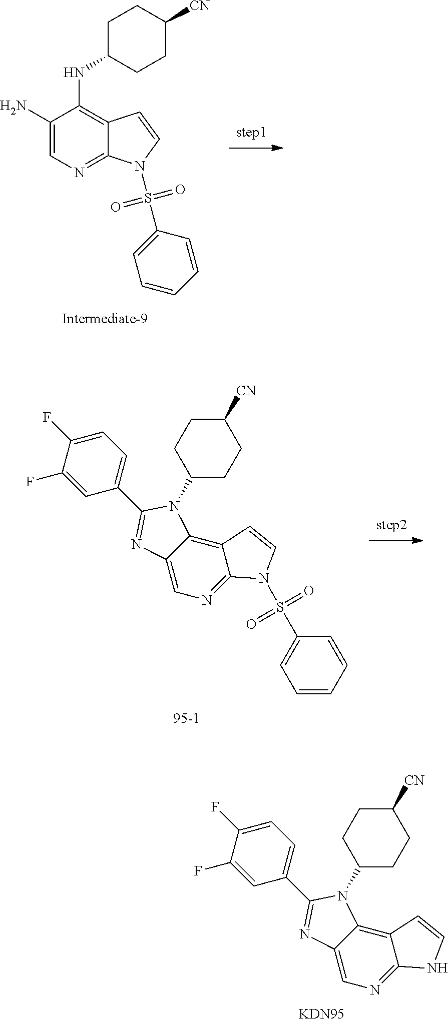

Step 1; Dissolve trans-4-((5-amino-1-(phenylsulfonyl)-1H-pyrrolo[2,3-b]pyridin-4-yl)amino)cyclohexanecarbonitrile (0.3 g, 0.8 mmol) in 10 mL of DMF, add Na₂S₂O₅ (0.8 g, 4 mmol) followed by the addition of 3,4-difluorobenzaldehyde (0.2 g, 1.6 mmol) dropwise. Stir for 12 h at 90° C. The reaction was monitored for completion by TLC. Saturated sodium bicarbonate was added until the reaction solution was weakly basic, and the organic phase was separated. The aqueous phase was extracted twice with dichloromethane. The organic phases were combined and washed with saturated saline, dried over anhydrous sodium sulfate and concentrated under vacuum to yield trans-4-(2-(3,4-difluorophenyl)-6-(phenylsulfonyl)imidazo[4,5-d]pyrrolo[2,3-b]pyridin-1(6H)-yl)cyclohexanecarbonitrile as a yellow oil (0.3 g, 76%). HRMS (ESI): m/z [M+H]⁺. C₂₇H₂₂F₂N₅O₂S calculated value 518.1457, measured value 518.1461.

Step 2: Dissolve trans-4-(2-(3,4-difluorophenyl)-6-(phenylsulfonyl)imidazo[4,5-d]pyrrolo[2,3-b]pyridin-1(6H)-yl)cyclohexanecarbonitrile (0.3 g, 0.6 mmol) in a mixture of 5 mL of tetrahydrofuran and 5 mL of methanol. After adding 5 mL of 1 M sodium hydroxide, stir at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum. The residue was purified using a silica gel column (petroleum ether:ethyl acetate=1:1) to yield LXS95: trans-4-(2-(3,4-difluorophenyl)imidazo[4,5-d]pyrrolo[2,3-b]pyridin-1(6H)-yl)cyclohexanecarbonitrile (0.1 g, 46%). ¹HNMR (300 MHz, DMSO-d₆): δ=12.02 (s, 1H), 8.66 (s, 1H), 7.77-7.84 (m, 1H), 7.56-7.73 (m, 1H), 7.55 (d, J=3.0 Hz, 2H), 6.88 (s, 1H), 4.414.49 (m, 1H), 3.15 (t, J=12.0 Hz, 1H), 2.32-2.44 (m, 2H), 2.23 (d, J=12.0 Hz, 2H), 2.04 (d, J=12.0 Hz, 2H), 1.73-1.85 (m, 2H) ppm; ¹³CNMR (75 MHz, DMSO-d₆) δ 153.78, 150.05, 149.64, 148.69, 142.19, 129.05, 127.83, 127.16, 124.74, 122.75, 120.75, 117.52, 115.63, 115.18, 97.58, 65.94, 31.89, 28.78, 22.63 ppm; HRMS (ESI): m/z [M+H]⁺. C₂₁H₁₈F₂N₅ calculated value 378.1525, measured value 378.1529.

### Example 96

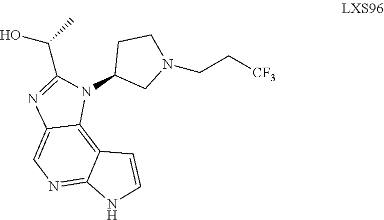 [SMILES: C[C@@H](O)c1nc2cnc3[nH]ccc3c2n1[C@H]1CCN(CCC(F)(F)F)C1]

### (R)-1-(1-((S)-1-(3,3,3-Trifluoropropyl)pyrrolidin-3-yl)imidazo[4,5-d]pyrrolo[2,3-b]pyridin-2-yl)ethanol

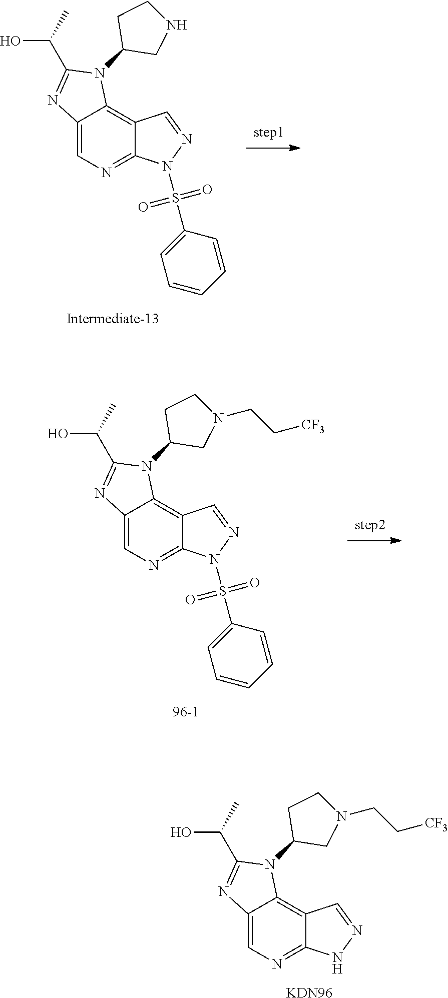

Step 1: Dissolve (R)-1-(6-(phenylsulfonyl)-1-((S)-pyrrolidin-3-yl)imidazo[4,5-d]pyrrolo[3,4-b]pyridin-2-yl)ethanol (0.3 g, 0.7 mmol) in 10 mL of tetrahydrofuran, add DIPEA (0.2 g, 1.4 mmol) followed by the slow dropwise addition of 4,4,4-trifluoro-1-iodopropane (0.2 g, 1.4 mmol). Raise the temperature to maintain reflux and stir for 3 h. Monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield (R)-1-(6-(phenylsulfonyl)-1-((S)-1-(3,3,3-trifluoropropyl)pyrrolidin-3-yl)imidazo[4,5-d]pyrrolo[2,3-b]pyridin-2-yl)ethanol as a pale yellow oil (0.2 g, 54%). The product can be used directly in the next step without further purification. HRMS (ESI): m/z [M+H]⁺. C₂₃H₂₅F₃N₅O₃S calculated value 508.1625, measured value 508.1630.

Step 2: Dissolve (R)-1-(6-(phenylsulfonyl)-1-((S)-1-(3,3,3-trifluoropropyl)pyrrolidin-3-yl)imidazo[4,5-d]pyrrolo[2,3-b]pyridin-2-yl)ethanol (0.2 g, 0.4 mmol) in a solvent mixture of 5 mL tetrahydrofuran and 5 mL methanol. After the addition of 5 mL of 1M sodium hydroxide, stir the mixture for 5 h at room temperature and monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS96: (R)-1-(1-((S)-1-(3,3,3-trifluoropropyl)pyrrolidin-3-yl)imidazo[4,5-d]pyrrolo[2,3-b]pyridin-2-yl)ethanol (0.1 g, 69%). ¹HNMR (300 MHz, DMSO-d₆): δ=12.09 (s, 1H), 8.87 (s, 1H), 7.53 (s, 1H), 6.83 (s, 1H), 4.60-4.75 (m, 1H), 3.73-3.78 (m, 1H), 2.56-2.87 (m, 2H), 2.42 (t, J=4.5 Hz, 2H), 2.15-2.32 (m, 2H), 1.90-2.11 (m, 2H), 1.41 (d, J=6.0 Hz, 3H), 1.35-1.40 (m, 2H) ppm; ¹³CNMR (75 MHz, DMSO-d₆) δ 148.67, 148.51, 142.18, 129.02, 127.11, 124.88, 120.74, 115.65, 99.35, 63.64, 58.94, 58.41, 55.95, 40.25, 38.45, 27.69, 22.81 ppm: HRMS (ESI): m/z [M+H]⁺. C₁₇H₂₁F₃N₅O calculated value 368.1693, measured value 368.1670.

### Example 97

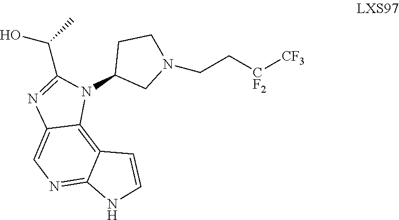 [SMILES: C[C@@H](O)c1nc2cnc3[nH]ccc3c2n1[C@H]1CCN(CCC(F)(F)C(F)(F)F)C1]

### (R)-1-(1-((S)-1-(3,3,4,4,4-Pentafluorobutyl)pyrrolidin-3-yl)imidazo[4,5-d]pyrrolo[2,3-b]pyridin-2-yl)ethanol

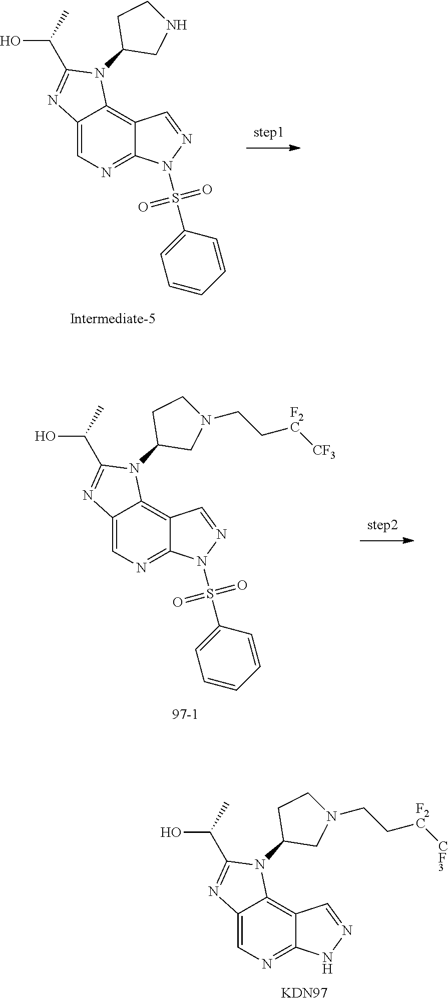

Step 1: Dissolve (R)-1-(6-(phenylsulfonyl)-1-((S)-pyrrolidin-3-yl)imidazo[4,5-d]pyrrolo[3,4-b]pyridin-2-yl)ethanol (0.3 g, 0.7 mmol) in 10 mL of tetrahydrofuran, add DIPEA (0.2 g, 1.4 mmol) and then slowly add 1,1,1,2,2-pentafluoro-4-iodobutane (0.3 g, 1.1 mmol) dropwise. Then, raise the temperature to maintain reflux and stir for 3 h. Monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield (R)-1-(1-((S)-1-(3,3,4,4,4-pentafluorobutyl)pyrrolidin-3-yl)-6-(phenylsulfonyl)imidazo[4,5-d]pyrrolo[2,3-b]pyridin-2-yl) ethanol as a pale yellow oil (0.2 g, 49%). The product can be used directly in the next step without further purification. HRMS (ESI): m/z [M+H]⁺. C₂₄H₂₅F₅N₅O₃S calculated value 558.1593, measured value 558.1602.

Step 2: Dissolve (R)-1-(1-((S)-1-(3,3,4,4,4-pentafluorobutyl)pyrrolidin-3-yl)-6-(phenylsulfonyl)imidazo[4,5-d]pyrrolo[2,3-b]pyridin-2-yl)ethanol (0.2 g, 0.36 mmol) in a mixture of 5 mL tetrahydrofuran and 5 mL methanol, and 5 mL of 1M sodium hydroxide was added and the mixture was then stirred at room temperature for 5 h. The reaction was monitored for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum, and the residue was purified using a silica gel column (petroleum ether:ethyl acetate=1:1) to yield LXS97: (R)-1-(1-((S)-1-(3,3,4,4,4-pentafluorobutyl)pyrrolidin-3-yl)imidazo[4,5-d]pyrrolo[2,3-b]pyridin-2-yl) ethanol (0.1 g, 67%). ¹HNMR (300 MHz, DMSO-d₆): δ=12.85 (s, 1H), 8.77 (s, 1H), 7.56 (s, 1H), 6.89 (s, 1H), 4.60-4.75 (m, 1H), 3.73-3.78 (m, 1H), 2.56-2.87 (m, 2H), 2.47 (t, J=4.5 Hz, 2H), 2.15-2.32 (m, 2H), 1.90-2.11 (m, 2H), 1.45 (d, J=6.0 Hz, 3H), 1.34-1.40 (m, 2H) ppm; ¹³CNMR (75 MHz, DMSO-d₆) δ 148.67, 148.51, 142.18, 131.88, 129.02, 127.11, 120.93, 119.74, 114.65, 97.35, 68.64, 53.94, 51.41, 50.95, 41.55, 37.40, 22.68, 21.40 ppm; HRMS (ESI): m/z [M+H]⁺. C₁₈H₂₁F₅N₅O calculated value 418.1661, measured value 418.1666.

### Example 98

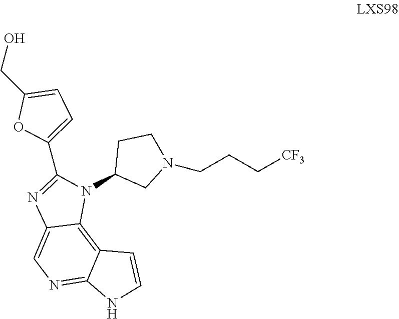 [SMILES: OCc1ccc(-c2nc3cnc4[nH]ccc4c3n2[C@H]2CCN(CCCC(F)(F)F)C2)o1]

### (S)-(5-(1-(1-(4,4,4-trifluorobutyl)pyrrolidin-3-yl)-1,6-dihydroimidazo[4,5-d]pyrrolo[2,3-b]pyridin-2-yl)furan-2-yl)methanol

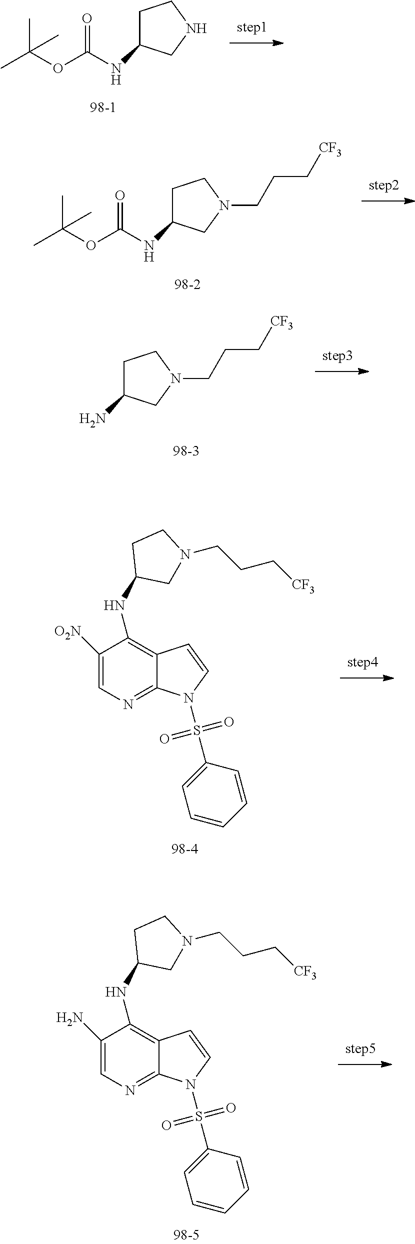 [SMILES: CC(C)(C)OC(=O)N[C@H]1CCNC1 ; CC(C)(C)OC(=O)N[C@H]1CCN(CCCC(F)(F)F)C1 ; N[C@H]1CCN(CCCC(F)(F)F)C1 ; O=[N+]([O-])c1cnc2c(ccn2S(=O)(=O)c2ccccc2)c1N[C@H]1CCN(CCCC(F)(F)F)C1 ; Nc1cnc2c(ccn2S(=O)(=O)c2ccccc2)c1N[C@H]1CCN(CCCC(F)(F)F)C1]

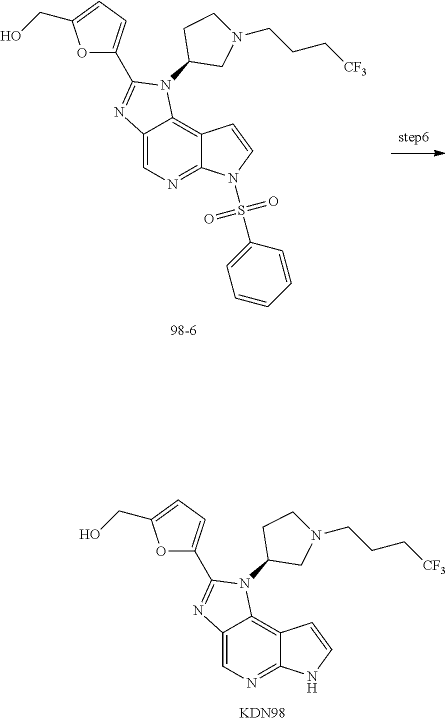

Step 1: Dissolve 98-1 (0.5 g, 2.7 mmol) in 10 mL of DMF, add K₂CO₃ (0.7 g, 5.4 mmol) followed by the slow dropwise addition of 4,4,4-trifluoro-1-iodobutane (0.7 g, 3.0 mmol), and then stir at room temperature for 12 h. Monitor the reaction for completion by MS. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield 98-2 as a white oil (0.7 g, 88%). The product was used directly in the next step without further purification. HRMS (ESI): m/z [M+H]⁺. C₁₃H₂₄F₃N₂O₂ calculated value 297.1784, measured value 297.1788.

Step 2: Dissolve 98-2 (0.7 g, 2.4 mmol) in 10 mL of dichloromethane, add trifluoroacetic acid (2.7 g, 24.0 mmol) and stir at room temperature for 12 h. Monitor the reaction for completion by MS. The product was concentrated under vacuum to obtain 98-3 as a light yellow oil (0.4 g, 99%). The product can be used directly in the next step without further purification. HRMS (ESI): m/z [M+H]⁺. C₈H₁₆F₃N₂ calculated value 197.1260, measured value 197.1266.

Step 3: Dissolve 98-3 (0.4 g, 2.0 mmol) in 10 mL of tetrahydrofuran, add DIPEA (0.5 g, 4.0 mmol) followed by the addition of 4-chloro-5-nitro-1-(phenylsulfonyl)-1H-pyrrolo[2,3-b]pyridine (0.6 g, 1.8 mmol). Raise the temperature to maintain reflux and stir for 3 h. Monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield 98-4 as a yellow oil (0.6 g, 68%). The product was used directly in the next step without further purification. HRMS (EST): m/z [M+H]⁺. C₂₁H₂₃F₃N₅O₄S calculated value 498.1417, measured value 498.1420.

Step 4: Dissolve 98-4 (0.6 g, 1.2 mmol) in 10 mL of methanol, add palladium carbon (0.1 g, 10%) and then use hydrogen to replace the air in the reaction flask more than three times. Keep the reaction in hydrogen atmosphere, stir for 12 h at room temperature, and monitor the reaction for completion by TLC. After filtration, the filtrate was collected and concentrated under vacuum to obtain 98-5 as a white foamy solid (0.5 g, 89%). The product can be used directly in the next step without further purification. HRMS (ESI): m/z [M+H]⁺. C₂₁H₂₅F₃N₅O₂S calculated value 468.1676, measured value 468.1680.

Step 5: Dissolve 98-5 (0.5 g, 1.8 mmol) in 10 mL of DMF, add Na₂S₂O₅ (0.7 g, 3.6 mmol) followed by the addition of 5-hydroxymethyl furfural (0.4 g, 3.2 mmol) dropwise, raise the temperature to 90° C. and stir for 12 h. Monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried over anhydrous sodium sulfate and concentrated under vacuum to yield 98-6 as a yellow oil (0.3 g, 49%). HRMS (ESI): m/z [M+H]⁺. C₂₇H₂₇F₃N₅O₄S calculated value 574.1730, measured value 574.1739.

Step 6: Dissolve 98-6 (0.2 g, 0.3 mmol) in a solvent mixture of 5 mL tetrahydrofuran and 5 mL methanol, add 5 mL of 1M sodium hydroxide and then stir at room temperature for 5 h. Monitor the reaction for completion by TLC. Saturated sodium bicarbonate was added until the reaction solution was weakly basic, and the organic phase was separated. The aqueous phase was extracted twice using dichloromethane. The organic phases were combined and washed with saturated saline, dried with anhydrous sodium sulfate and concentrated under vacuum, and the residue was purified using a silica gel column (petroleum ether:ethyl acetate =1:1) to yield LXS98: (S)-(5-(1-(1-(4,4,4-trifluorobutyl)pyrrolidin-3-yl)-1,6-dihydroimidazo[4,5-d]pyrrolo[2,3-b]pyridin-2-yl)furan-2-yl)methanol (0.1 g, 66%). ¹HNMR (300 MHz, DMSO-d₆): δ=12.98 (s, 1H), 8.87 (s, 1H), 7.51 (s, 1H), 7.02 (d, J=6.0 Hz, 1H), 6.84 (s, 1H), 6.59 (d, J=6.0 Hz, 1H), 4.39 (s, 1H), 3.79-3.88 (m, 1H), 2.53-2.81 (m, 2H), 2.43 (t, J=6.0 Hz, 2H), 2.20-2.30 (m, 2H), 1.90-2.15 (m, 2H), 1.81 (d, J=6.0 Hz, 2H), 1.36 (m, 2H) ppm; ¹³CNMR (75 MHz, DMSO-d₆) δ 153.84, 151.12, 148.62, 144.95, 142.19, 129.08, 127.15, 126.84, 120.74, 115.64, 107.95, 104.08, 99.35, 57.85, 57.34, 56.38, 55.97, 55.84, 37.75, 27.08, 10.18 ppm; HRMS (ESI): m/z [M+H]⁺. C₂₁H₂₃F₃N₅O₂ calculated value 434.1798, measured value 434.1788.

### Example 99

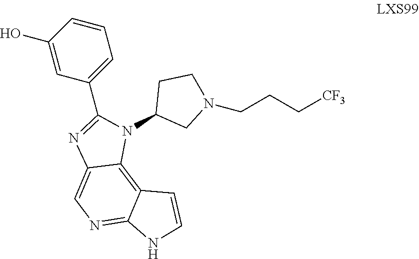 [SMILES: Oc1cccc(-c2nc3cnc4[nH]ccc4c3n2[C@H]2CCN(CCCC(F)(F)F)C2)c1]

### (S)-3-(1-(1-(4,4,4-trifluorobutyl)pyrrolidin-3-yl)-1,6-dihydroimidazo[4,5-d]pyrrolo[2,3-b]pyridin-2-yl)phenol

LXS99 was prepared using the raw material of 3-hydroxybenzaldehyde in a similar preparation way as described in Example 98, and the crude product was purified using a silica gel column (petroleum ether:ethyl acetate=1:1) to yield LXS99: (S)-3-(1-(1-(4,4,4-trifluorobutyl)pyrrolidin-3-yl)-1,6-dihydroimidazo[4,5-d]pyrrolo[2,3-b]pyridin-2-yl)phenol (0.1 g, 54%). ¹HNMR (300 MHz, DMSO-d₆): δ=12.17 (s, 1H), 8.89 (s, 1H), 7.84 (t, J=3.0 Hz, 1H), 7.58 (s, 1H), 7.34 (t, J=7.5 Hz, 1H), 7.04 (s, 1H), 6.90-7.02 (m, 1H), 6.91 (d, J=3.0 Hz, 1H), 6.08 (d, J=7.5 Hz, 1H), 3.79-3.81 (m, 2H), 2.50-2.80 (m, 2H), 2.45 (t, J=6.0 Hz, 2H), 2.21-2.37 (m, 2H), 1.93-2.19 (m, 2H), 1.83 (d, J=6.0 Hz, 2H), 1.34 (m, 2H) ppm; ¹³CNMR (75 MHz, DMSO-d₆) δ 157.54, 153.74, 148.62, 142.18, 132.08, 130.68, 129.08, 127.10, 126.85, 120.77, 120.65, 115.95, 115.64, 112.97, 99.34, 58.46, 58.21, 56.37, 55.94, 37.77, 27.62, 10.18 ppm; HRMS (ESI): m/z [M+H]⁺. C₂₂H₂₃F₃N₅O calculated value 430.1849, measured value 430.1933.

### Example 100

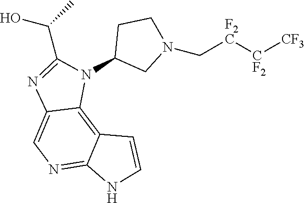 [SMILES: C[C@@H](O)c1nc2cnc3[nH]ccc3c2n1[C@H]1CCN(CC(F)(F)C(F)(F)C(F)(F)F)C1]

### (R)-1-(1-((S)-1-(2,2,3,3,4,4,4-Heptafluorobutyl)pyrrolidin-3-yl)imidazo[4,5-d]pyrrolo[2,3-b]pyridin-2-yl)ethanol

LXS100 was prepared using a preparation method similar to Example 97 with the raw material of 2,2,3,3,4,4,4-heptafluoroiodobutane, and the crude product was purified using a silica gel column (petroleum ether:ethyl acetate=1:1) to yield LXS100: (R)-1-(1-((S)-1-(2,2,3,3,4,4,4-Heptafluorobutyl)pyrrolidin-3-yl)imidazo[4,5-d]pyrrolo[2,3-b]pyridin-2-yl)ethanol (0.1 g, 43%). ¹HNMR (300 MHz, DMSO-d₆): δ=12.35 (s, 1H), 8.87 (s, 1H), 7.54 (s, 1H), 6.63 (s, 1H), 4.60-4.70 (m, 1H), 3.73-3.79 (m, 1H), 2.56-2.81 (m, 2H), 2.68 (s, 2H), 2.20-2.30 (m, 2H), 1.90-2.15 (m, 2H), 1.49 (d, J=6.0 Hz, 3H) ppm; ¹³ C NMR (75 MHz, DMSO-d₆) δ 148.79, 148.59, 129.16, 127.19, 120.88, 120.71, 118.74, 115.65, 104.35, 99.65, 63.64, 58.94, 58.41, 55.91, 51.95, 27.55, 22.40 ppm; HRMS (ESI): m/z [M+H]⁺. C₁₈H₁₉F₇N₅O calculated value 454.1472, measured value 454.1488.

### Example 101

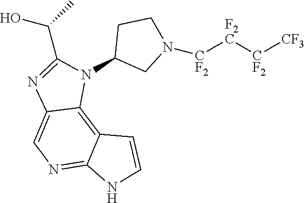 [SMILES: C[C@@H](O)c1nc2cnc3[nH]ccc3c2n1[C@H]1CCN(C(F)(F)C(F)(F)C(F)(F)C(F)(F)F)C1]

(R)-1-(1-((S)-1-(Perfluoro-butyl)pyrrolidin-3-yl)-1,6-dihydroimidazo[4,5-d]pyrrolo[2,3-b]pyridin-2-yl)ethanolLXS101 was prepared using a similar preparation method as in Example 97, with the raw material of perfluorobutane. The crude product was purified using a silica gel column (petroleum ether:ethyl acetate=1:1) to yield LXS101: (R)-1-(1-((S)-1-(Perfluoro-butyl)pyrrolidin-3-yl)-1,6-dihydroimidazo[4,5-d]pyrrolo[2,3-b]pyridin-2-yl)ethanol (0.1 g, 64%). ¹HNMR (300 MHz, DMSO-d₆): δ=12.45 (s, 1H), 8.91 (s, 1H), 7.39 (s, 1H), 6.68 (s, 1H), 4.60-4.74 (m, 1H), 3.73-3.82 (m, 1H), 2.54-2.88 (m, 2H), 1.90-2.30 (m, 4H), 1.51 (d, J=6.0 Hz, 3H) ppm; ¹³CNMR (75 MHz, DMSO-d₆) δ 156.61, 148.61, 148.51, 142.18, 129.01, 127.34, 120.78, 118.20, 115.28, 107.24, 99.33, 63.88, 58.99, 44.84, 42.38, 27.65, 22.81 ppm; HRMS (ESI): m/z [M+H]⁺. C₁₈H₁₇F₉N₅O calculated value 490.1284, measured value 490.1288.

### Example 102

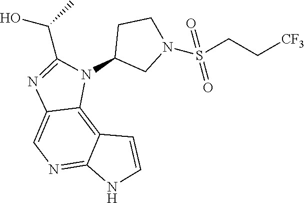 [SMILES: C[C@@H](O)c1nc2cnc3[nH]ccc3c2n1[C@H]1CCN(S(=O)(=O)CCC(F)(F)F)C1]

### (R)-1-(1-((S)-1-((3,3,3-Trifluoropropyl)sulfonyl)pyrrolidin-3-yl)imidazo[4,5-d]pyrrolo[2,3-b]pyridin-2-yl)ethanol

(i) LXS102 was prepared using a similar preparation method as in Example 97 using the raw material of trifluoromethylpropylsulfonyl chloride, and the crude product was purified using a silica gel column (petroleum ether:ethyl acetate=1:1) to yield LXS102: (R)-1-(1-((S)-1-((3,3,3-trifluoropropyl)sulfonyl)pyrrolidin-3-yl)imidazo[4,5-d]pyrrolo[2,3-b]pyridin-2-yl) ethanol (0.2 g, 69%). ¹HNMR (300 MHz, DMSO-d₆): δ=12.71 (s, 1H), 8.56 (s, 1H), 7.38 (s, 1H), 6.74 (s, 1H), 4.68-4.78 (m, 1H), 3.73-3.84 (m, 1H), 3.20 (d, J=4.5 Hz, 2H), 3.11 (t, J=6.0 Hz, 2H), 2.70-2.84 (m, 2H), 2.37 (t, J=6.0 Hz, 2H), 1.91-2.15 (m, 2H), 1.43 (d, J=6.0 Hz, 3H) ppm; ¹³CNMR (75 MHz, DMSO-d₆) δ 148.66, 148.51, 142.15, 129.05, 127.10, 125.47, 120.71, 115.64, 99.34, 63.64, 57.58, 56.14, 50.04, 40.08, 29.87, 26.27, 22.87 ppm; HRMS (ESI); m/z [M+H]⁺. C₁₇H₂₁F₃N₅O₃S calculated value 432.1312, measured value 432.1318.

### Example 103

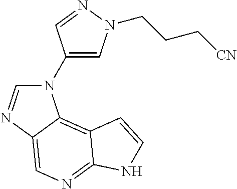 [SMILES: N#CCCCn1cc(-n2cnc3cnc4[nH]ccc4c32)cn1]

### 4-(4-(Imidazo[4,5-d]pyrrolo[2,3-b]pyridin-1(6H)-yl)-1H-pyrazol-1-yl)butanenitrile

LXS103 was prepared using a preparation method similar to that used for the preparation of Intermediate 10 and Example 79 using the raw material of 4-bromopropyl cyanide, and the crude product was purified using a silica gel column (petroleum ether:ethyl acetate=1:1) to yield LXS103: 4-(4-(imidazo[4,5-d]pyrrolo[2,3-b]pyridin-1(6H)-yl)-1H-pyrazol-1-yl)butanenitrile (0.1 g, 57%). ¹ HNMR (300 MHz, DMSO-d₆): δ=12.13 (s, 1H), 8.88 (s, 1H), 8.06 (s, 1H), 7.94 (s, 1H), 7.55 (s, 1H), 7.15 (s, 1H), 6.68 (s, 1H), 4.46 (t, J=6.0 Hz, 2H), 2.10-2.21 (m, 2H), 1.87 (t, J=6.0 Hz, 2H) ppm; ¹³ C NMR (75 MHz, DMSO-d₆) δ 148.77, 142.11, 134.58, 130.77, 129.71, 129.08, 127.88, 120.74, 119.34, 115.67, 100.57, 99.37, 52.38, 22.74, 14.97 ppm; HRMS (ESI): m/z [M+H]⁺. C₁₈H₁₄N₇ calculated value 292.1305, measured value 292.1316.

### Example 104

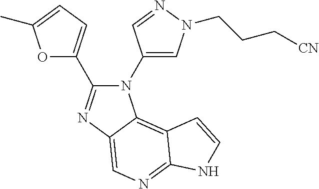 [SMILES: Cc1ccc(-c2nc3cnc4[nH]ccc4c3n2-c2cnn(CCCC#N)c2)o1]

### 4-(4-(2-(5-methylfuran-2-yl)imidazo[4,5-d]pyrrolo[2,3-b]pyridin-1(6H)-yl)-1H-pyrazol-1-yl)butanenitrile

LXS104 was prepared using a similar preparation method as in Example 103 using the raw material of 5-methylfuran-2-formaldehyde, and the crude product was purified using a silica gel column (petroleum ether:ethyl acetate=1:1) to yield LXS104: 4-(4-(2-(5-methylfuran-2-yl)imidazo[4,5-d]pyrrolo[2,3-b]pyridin-1(6H)-yl)-1H-pyrazol-1-yl)butanenitrile (0.2 g, 67%). ¹ HNMR (300 MHz, DMSO-d₆): δ=12.33 (s, 1H), 8.85 (s, 1H), 8.11 (s, 1H), 7.98 (s, 1H), 7.55 (s, 1H), 6.95 (d, J=6.0 Hz, 1H), 6.68 (s, 1H), 6.08 (d, J=6.0 Hz, 1H), 4.38 (t, J=6.0 Hz, 2H), 2.30 (s, 3H), 2.14-2.25 (m, 2H), 1.91 (t, J=4.5 Hz, 2H) ppm; ¹³C NMR (75 MHz, DMSO-d₆) δ 152.21, 151.47, 148.65, 142.18, 141.28, 130.75, 129.70, 129.08, 127.18, 120.78, 119.58, 115.68, 107.88, 107.67, 100.51, 99.34, 52.38, 22.74, 14.95, 13.77 ppm; HRMS (ESI): m/z [M+H]⁺. C₂₀H₁₈N₇O calculated value 372.1567, measured value 352.1588.

### Example 105

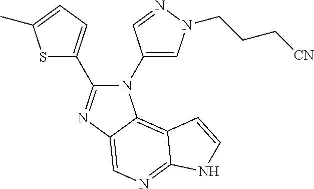 [SMILES: Cc1ccc(-c2nc3cnc4[nH]ccc4c3n2-c2cnn(CCCC#N)c2)s1]

### 4-(4-(2-(5-Methylthiophen-2-yl)imidazo[4,5-d]pyrrolo[2,3-b]pyridin-1(6H)-yl)-1H-pyrazol-1-yl butanenitrile

LXS105 was prepared using a preparation method similar to Example 103 using the raw material of 5-methylthiophene-2-formaldehyde, and the crude product was purified using a silica gel column (petroleum ether:ethyl acetate=1:1) to yield LXS105: 4-(4-(2-(5-methylthiophen-2-yl)imidazo[4,5-d]pyrrolo[2,3-b]pyridin-1(6H)-yl)-1H-pyrazol-1-yl butanenitrile (0.1 g, 45%). ¹ HNMR (300 MHz, DMSO-d₆): δ=12.28 (s, 1H), 8.68 (s, 1H), 8.06 (s, 1H), 7.94 (s, 1H), 7.63 (s, 1H), 7.49 (d, J=6.0 Hz, 1H), 6.83 (d, J=6.0 Hz, 1H), 6.61 (s, 1H), 4.46 (t, J=6.0 Hz, 2H), 2.36 (s, 3H), 2.11-2.23 (m, 2H), 1.87 (t, J=4.5 Hz, 2H) ppm; ¹³ C NMR (75 MHz, DMSO-d₆) δ 148.66, 142.18, 141.68, 141.24, 134.38, 130.75, 129.75, 129.04, 127.54, 127.45, 120.77, 119.35, 115.67, 100.57, 99.35, 52.38, 22.75, 15.29, 14.95 ppm; HRMS (ESI): m/z [M+H]⁺. C₂₀H₁₈N₇S calculated value 388.1339, measured value 388.1345.

### Example 106

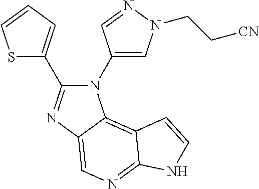 [SMILES: N#CCCn1cc(-n2c(-c3cccs3)nc3cnc4[nH]ccc4c32)cn1]

### (4-(2-(Thiophen-2-yl)imidazo[4,5-d]pyrrolo[2,3-b]pyridin-1(6H)-yl)-1H-pyrazol-1-yl)propanenitrile

LXS106 was prepared using a similar preparation method as in Example 79 using the raw material of thiophene-2formaldehyde, and the crude product was purified using a silica gel column (petroleum ether:ethyl acetate=1:1) to yield LXS106: 3-(4-(2-(Thiophen-2-yl)imidazo[4,5-d]pyrrolo[2,3-b]pyridin-1(6H)-yl)-1H-pyrazol-1-yl)propanenitrile (0.1 g, 64%). ¹ HNMR (300 MHz, DMSO-d₆): δ=12.34 (s, 1H), 8.84 (s, 1H), 8.34 (s, 1H), 7.98 (s, 1H), 7.85 (d, J=4.5 Hz, 1H), 7.69 (d, J=4.5 Hz, 1H), 7.63 (s, 1H), 7.19 (m, 1H), 6.56 (s, 1H), 5.08 (t, J=6.0 Hz, 2H), 3.20 (t, J=6.0 Hz, 2H) ppm; ¹³ C NMR (75 MHz, DMSO-d₆) δ 148.68, 143.98, 142.18, 141.28, 130.74, 129.72, 129.10, 129.00, 128.61, 128.07, 127.15, 120.75, 117.77, 115.65, 100.58, 99.38, 49.25, 15.92 ppm: HRMS (ES1): m/z [M+H]⁺. C₁₈H₁₄N₇S calculated value 360.1026, measured value 360.1038.

### Example 107

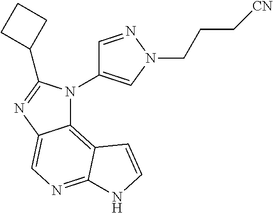 [SMILES: N#CCCCn1cc(-n2c(C3CCC3)nc3cnc4[nH]ccc4c32)cn1]

### 4-(4-(2-Cyclobutylimidazo[4,5-d]pyrrolo[2,3-b]pyridin-1(6H)-yl)-1H-pyrazol-1-yl)butanenitrile

LXS107 was prepared using a similar preparation method as in Example 103 using the raw material of cyclobutanecarboxaldehyde, and the crude product was purified using a silica gel column (petroleum ether:ethyl acetate=1:1) to yield LXS107: 4-(4-(2-cyclobutylimidazo[4,5-d]pyrrolo[2,3-b]pyridin-1(6H)-yl)-1H-pyrazol-1-yl)butanenitrile (0.1 g, 57%). ¹ HNMR (300 MHz, DMSO-d₆): δ=12.34 (s, 1H), 8.89 (s, 1H), 8.06 (s, 1H), 7.96 (s, 1H), 7.63 (s, 1H), 6.56 (s, 1H), 4.46 (t, J=6.0 Hz, 2H), 3.19-3.24 (m, 1H), 2.15-2.38 (m, 4H), 2.10-2.14 (m, 2H), 1.91-2.01 (m, 2H), 1.77-1.87 (m, 2H) ppm; ¹³ C NMR (75 MHz, DMSO-d₆) δ 148.60, 144.89, 142.18, 130.77, 129.71, 129.08, 127.18, 120.75, 119.37, 115.67, 100.59, 99.38, 52.38, 32.69, 26.28, 22.78, 18.93, 14.92 ppm; HRMS (ESI): m/z [M+H]⁺. C₁₉H₂₀N₇ calculated value 346.1775, measured value 346.1778.

### Example 108

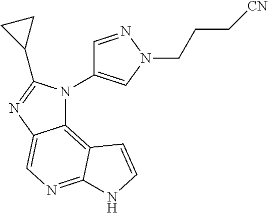 [SMILES: N#CCCCn1cc(-n2c(C3CC3)nc3cnc4[nH]ccc4c32)cn1]

### 4-(4-(2-Cyclopropylimidazo[4,5-d]pyrrolo[2,3-b]pyridin-1(6H)-yl)-1H-pyrazol-1-yl)butanenitrile

LXS108 was prepared using a similar preparation method as in Example 103 using the raw material of cyclopropylformaldehyde, and the crude product was purified using a silica gel column (petroleum ether:ethyl acetate=1:1) to yield LXS108: 4-(4-(2-cyclopropylimidazo[4,5-d]pyrrolo[2,3-b]pyridin-1(6H)-yl)-1H-pyrazol-1-yl)butanenitrile (0.1 g, 62%). ¹ HNMR (300 MHz, DMSO-d₆): δ=12.11 (s, 1H), 8.69 (s, 1H), 8.11 (s, 1H), 7.94 (s, 1H), 7.58 (s, 1H), 6.41 (s, 1H), 4.51 (t, J=6.0 Hz, 2H), 2.10-2.18 (m, 2H), 1.77-1.87 (m, 2H), 1.35-1.52 (m, 1H), 0.99-1.24 (m, 4H) ppm; ¹³ C NMR (75 MHz, DMSO-d₆) δ 148.68, 144.51, 142.98, 130.77, 129.75, 129.05, 127.15, 120.77, 119.35, 115.68, 100.54, 99.35, 52.38, 22.74, 14.95, 8.59, 3.48 ppm; HRMS (ESI): m/z [M+H]⁺. C₁₈H₁₈N₇ calculated value 332.1618, measured value 332.1624.

### In Vitro Activity Test of JAK1 and JAK2 Kinases

### Experimental Materials

Reagents and consumables

| Reagent name | Supplier | Item No. | Lot number |
|---|---|---|---|
| JAK1 | Carna | 08-114 | 11CBS-0144M |
| JAK2 | Carna | 08-045 | 10CBS-0289R |
| Kinase substrate22 | GL | 112393 | P190917-CL112393 |
| Kinase JAK1 peptide | GL | 758318 | P191104-TL758318 |
| DMSO | Sigma | D8418-1L | SHBG3288V |
| 384-well plate | Corning | 3573 | 12619003 |

### Experimental Apparatus

Centrifuge (manufacturer: Eppendorf, model: 5430)
ELISA Instrument (Manufacturer: Perkin Elmer, Model: Caliper EZ Reader 1I)
Echo 550 (Manufacturer: Labcyte, Model: Echo 550)

### Experimental Method

### 1. Kinase Reaction Process

(1) Prepare 1 Kinase buffer.

(2) Compound concentration gradient preparation: The test compound was tested at 1000 nM, 3-fold dilution, 10 concentrations, two-well assay, and configured to a 100-fold final concentration in a 384-well plate. 250 nl of the test compound was then transferred to the 384 reaction plate using Echo550. 250 nl of 100% DMSO was added to each of the negative and positive control wells.

(3) Prepare 2.5 times the final concentration of kinase solution with 1×Kinase buffer.

(4) Add 10 μL of 2.5 times the final concentration of kinase solution to the compound wells and 10 μL of 1×Kinase buffer to the negative control wells.

(5) Centrifuge at 1000 rpm for 30 seconds, mix well with shaking, and incubate at room temperature for 10 minutes.

(6) Prepare a 25/15-fold final concentration of mixture of ATP and Kinase substrate with 1×Kinase buffer.

(7) Add 15 μL of a mixture of 25/15 times the final concentration of ATP and substrate to start the reaction.

(8) Centrifuge the 384-well plate at 1000 rpm for 30 seconds, mix well with shaking, and incubate at room temperature for an appropriate amount of time.

(9) Stop the kinase reaction by adding 30 μL of termination solution, centrifuge at 1000 rpm for 30 seconds, and mix well with shaking.

(10) Read the conversion rate with Caliper EZ Reader.

### 2. Data Analysis

### Calculation Formula

$$\% Inhibition = \frac{Conversion \% \_max - Conversion \% \_sample}{Conversion \% \_max - Conversion \% \_min} \times 100$$

where: Conversion %_sample is the conversion rate reading of the sample-, Conversion %_min: the average value of the negative control wells, representing the conversion reading of the wells without enzyme activity: Conversion %-max: the average value of the positive control wells, representing the conversion reading of the wells without compound inhibition.

### Fitting the Dose-Effect Curve

The log value of concentration was used as the X-axis and the percentage inhibition as the Y-axis, and the function of the analytical software GraphPad Prism 5; log(inhibitor) vs. response -Variable slope, was used to lit dose-effect curves to obtain the IC50) values of each compound on enzyme activity.

The formula is Y=Bottom+(Top-Bottom)/(1+10\hat{ }((LogIC50-X)*HillSlope))

The 1C₅₀ data are specified in Table 1.

TABLE 1

| Example of | | | |
|---|---|---|---|
| implementation | Compound | JAK1 | JAK2 |
| 1 | LXS01 | C | C |
| 2 | LXS02 | C | C |
| 3 | LXS03 | C | C |
| 4 | LXS04 | B | C |
| 5 | LXS05 | B | C |
| 6 | LXS06 | B | C |
| 7 | LXS07 | C | C |
| 8 | LXS08 | C | C |
| 9 | LXS09 | C | D |
| 10 | LXS10 | C | D |
| 11 | LXS11 | B | C |
| 12 | LXS12 | B | C |
| 13 | LXS13 | C | B |
| 14 | LXS14 | C | B |
| 15 | LXS15 | C | B |
| 16 | LXS16 | B | B |
| 17 | LXS17 | B | B |
| 18 | LXS18 | B | B |
| 19 | LXS19 | B | B |
| 20 | LXS20 | C | C |
| 21 | LXS21 | C | C |
| 22 | LXS22 | B | B |
| 23 | LXS23 | C | C |
| 24 | LXS24 | C | C |
| 25 | LXS25 | C | C |
| 26 | LXS26 | B | B |
| 27 | LXS27 | B | B |
| 28 | LXS28 | B | B |
| 29 | LXS29 | B | B |
| 30 | LXS30 | C | C |
| 31 | LXS31 | C | C |
| 32 | LXS32 | B | A |
| 33 | LXS33 | B | B |
| 34 | LXS34 | C | C |
| 35 | LXS35 | B | A |
| 36 | LXS36 | B | B |
| 37 | LXS37 | C | C |
| 38 | LXS38 | B | B |
| 39 | LXS39 | B | C |
| 40 | LXS40 | B | B |
| 41 | LXS41 | C | C |
| 42 | LXS42 | C | C |
| 43 | LXS43 | B | C |
| 44 | LXS44 | B | B |
| 45 | LXS45 | B | B |
| 46 | LXS46 | B | A |
| 47 | LXS47 | C | C |
| 48 | LXS48 | B | C |
| 49 | LXS49 | B | C |
| 50 | LXS50 | C | A |
| 51 | LXS51 | B | B |
| 52 | LXS52 | C | B |
| 53 | LXSS3 | B | B |
| 54 | LXS54 | C | C |
| 55 | LXS55 | C | C |
| 56 | LXS56 | B | C |
| 57 | LXS57 | C | C |
| 58 | LXS58 | B | B |
| 59 | LXS59 | B | C |
| 60 | LXS60 | B | B |
| 61 | LXS61 | C | C |
| 62 | LXS62 | C | A |
| 63 | LXS63 | C | A |
| 64 | LXS64 | C | A |
| 65 | LXS65 | A | C |
| 66 | LXS66 | B | B |
| 67 | LXS67 | B | B |
| 68 | LXS68 | A | C |
| 69 | LXS69 | B | B |
| 70 | LXS70 | B | B |
| 71 | LXS71 | A | C |
| 72 | LXS72 | B | B |
| 73 | LXS73 | A | C |
| 74 | LXS74 | A | C |
| 75 | LXS75 | A | C |
| 76 | LXS76 | A | C |
| 77 | LXS77 | C | A |
| 78 | LXS78 | B | B |
| 79 | LXS79 | C | A |
| 80 | LXS80 | B | B |
| 81 | LXS81 | B | B |
| 82 | LXS82 | B | B |
| 83 | LXS83 | B | B |
| 84 | LXS84 | B | B |
| 85 | LXS85 | A | C |
| 86 | LXS86 | A | C |
| 87 | LXS87 | A | C |
| 88 | LXS88 | A | C |
| 89 | LXS89 | B | C |
| 90 | LXS90 | B | C |
| 91 | LXS91 | A | C |
| 92 | LXS92 | B | D |
| 93 | LXS93 | A | C |
| 94 | LXS94 | B | C |
| 95 | LXS95 | C | B |
| 96 | LXS96 | A | C |
| 97 | LXS97 | A | C |
| 98 | LXS98 | B | B |
| 99 | LXS99 | B | B |
| 100 | LXS100 | A | C |
| 101 | LXS101 | A | C |
| 102 | LXS102 | A | C |
| 103 | LXS103 | B | B |
| 104 | LXS104 | B | B |
| 105 | LXS105 | C | B |
| 106 | LXS106 | C | B |
| 107 | LXS107 | C | B |
| 108 | LXS108 | C | B |

A < 10 nM; 10 nM < B < 100 nM; 100 nM < C < 1000 nM; D > 1000 nM.

1. A compound shown in formula: 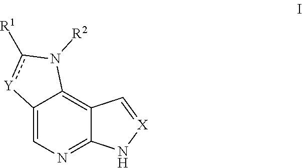
or a tautomer, stereoisomer, racemate or isotopic derivative thereof, or a pharmaceutically acceptable salt of any of the foregoing, or a crystal form or solvate of any of the foregoing;
wherein X is CH or N;
Y is NH or N; when Y is N, the [symbol] connected to Y is a double bond; when Y is NH, the [symbol] connected to Y is a single bond;
R¹ and R² are defined as (i), (ii) or (iii) as follows:
(i) R¹ is 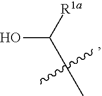
and R² is 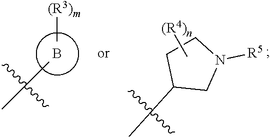
ring B is a benzene ring or a 5-6-membered heteroaromatic ring;
R^{1a} is a C₁₋₃ alkyl group;
each R³ is independently a halogen, a cyano group, a C₁₋₄ alkyl group, a C₁₋₄ haloalkyl group, a —O—C₁₋₄ alkyl group, or a —O—C₁₋₄ haloalkyl group;
each R⁴ is independently a halogen, a hydroxyl group, a C₁₋₄ alkyl group, a C₁₋₄ haloalkyl group, a —O—C₁₋₄ alkyl group, a —O—C₁₋₄ haloalkyl group or a C₁₋₄ hydroxyalkyl group;
alternatively, two R⁴ located on the same carbon atom or on different carbon atoms are interconnected to form —CH₂— or —(CH₂)₂—;
R⁵ is —S(O)₂R^{5a}, —C(O)R^{5b}, —C(O)NR^{5c}R^{5d}, —C(O)OR^{5e}, —C(O)NHR^{5k} or —L¹-R^{5f};
R^{5a} is a C₂₋₆ alkyl group, a C₂₋₆ haloalkyl group, -L¹-R^{5f}, an unsubstituted or substituted 6-10-membered aryl group or an unsubstituted or substituted 5-10-membered heteroaryl group, said substituted 6-10-membered aryl group and substituted 5-10-membered heteroaryl group are defined as structures where 1, 2, 3 or 4 hydrogen atoms in the 6-10-membered aryl group and the 5-10-membered heteroaryl group are independently substituted by R^{5g};
R^{5b}, R^{5c}, R^{5d}, R^{5e} and R^{5k} are each independently a C₁₋₆ alkyl group, a C₁₋₆ haloalkyl group, -L¹-R^{5f}, an unsubstituted or substituted 6-10-membered aryl group, or an unsubstituted or substituted 5-10-membered heteroaryl group, said substituted 6-10-membered aryl group and substituted 5-10-membered heteroaryl group are defined as structures where 1, 2, 3 or 4 hydrogen atoms in the 6-10-membered aryl group and the 5-10-membered heteroaryl group are independently substituted by R⁵;
each R^{5g} and R^{5h} is independently a halogen, a cyano group, a C₁₋₄ alkyl group, a C₁₋₄ haloalkyl group, a —O—C₁₋₄ alkyl group, a —O—C₁₋₄ haloalkyl group, or a phenyl group;
each L¹ is independently —[C(R^{a}R^{b})]₁₋₅—, —[C(R^{a}R^{b})]₁₋₂—C(O)—[C(R^{a}R^{b})]₁₋₂—, —[C(R^{a}R^{b})]₁₋₂—C(O)NH—[C(R^{a}R^{b})]₁₋₂—, —[C(R^{a}R^{b})]₁₋₂—NHC(O)—[C(R^{a}R^{b})]₁₋₂—, —[C(R^{a}R^{b})]₁₋₂—S(O)₂—[C(R^{a}R^{b})]¹⁻²—, —[C(R^{a}R^{b})]₁₋₂—NHS(O)₂—[C(R^{a}R^{b})]₁₋₂— or —[C(R^{a}R^{b})]₁₋₂—S(O)₂NH—[C(R^{a}R^{b})]₁₋₂;
each R^{5f}is independently H, F, CHF₂, CH₂F, CF₃ or CN;
each R^{a} is independently H, a halogen or a C₁₋₃ alkyl group;
each R^{b} is independently H, a halogen or a C₁₋₃ alkyl group;
alternatively, R^{a} and R^{b}, along with the carbon atom connected to them, form a cyclopropyl group;
m is 0, 1, 2, 3 or 4;
n is 0, 1, 2, 3 or 4;
(ii) R¹ is 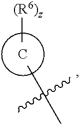
and R² is 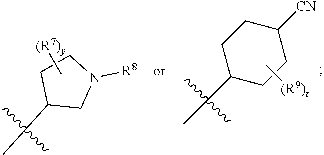
ring C is a benzene ring or a 5-6-membered heteroaromatic ring;
each R⁶ is independently a halogen, a hydroxyl group, an amino group, a cyano group, a C₁₋₄ alkyl group, a C₁₋₄ haloalkyl group, a —O—C₁₋₄ alkyl group, a —O—C₁₋₄ haloalkyl group, a —S—C₁₋₄ alkyl group, a —S(O)₂—C₁₋₄ alkyl group or a C₁₋₄ hydroxyalkyl group;
each R⁷ is independently R⁴;
alternatively, two R₇ located on the same carbon atom or on different carbon atoms are interconnected to form —CH₂— or —(CH₂)₂—;
R⁸ is R⁵, —S(O)₂R^{8a}, —C(O)R^{8b}, —C(O)NR^{8c}R^{8d}, —C(O)OR^{8e} or —C(O)NHR^{8k};
R^{8a}, R^{8b}, R^{8c}, R^{8d}, R^{8e} and R^{8k} are each independently a methyl group, —CF₃, a C₂₋₆ alkenyl group or a C₃₋₆ cycloalkyl group;
each R⁹ is independently R⁴;
alternatively, two R⁹ located on the same carbon atom or on different carbon atoms are interconnected to form —CH₂— or —(CH₂)₂—;
z is 0, 1, 2, 3 or 4;
y is 0, 1, 2, 3 or 4;
t is 0, 1, 2, 3 or 4;
(iii) R¹ is H, CF₃ or 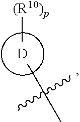
and R² is 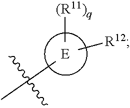
ring D is a C₃₋₆ cycloalkyl group, benzene ring or 5-6-membered heteroaromatic ring;
ring E is a benzene ring or a 5-6-membered heteroaromatic ring;
each R¹⁰ and R¹¹ is independently a halogen, a hydroxyl group, an amino group, a cyano group, a C₁₋₄ alkyl group, a C₁₋₄ haloalkyl group, a —O—C₁₋₄ alkyl group, a —O—C₁₋₄ haloalkyl group, a —S—C₁₋₄ alkyl group, a —S(O)₂—C₁₋₄ alkyl group or a C₁₋₄ hydroxyalkyl group;
R¹² is a cyano group, -L³-R^{12f} or 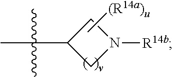
each R^{14a} is independently a halogen, a cyano group, a C₁₋₄ alkyl group, a cyano-substituted C₁₋₄ alkyl group, a C₁₋₄ haloalkyl group, a —O—C₁₋₄ alkyl group, a —O—C₁₋₄ haloalkyl group, or a C₁₋₄ hydroxyalkyl group;
R^{14b} is R⁵ or a —S(O)₂—C¹⁻⁴ alkyl group;
L³ is —[C(R^{e}R^{f})]₁₋₅—, —C(O)—, —C(O)NH—, —NHC(O)—, —S(O)₂—, —NHS(O)₂—, —S(O)₂NH—, —[C(R^{e}R^{f})]₁₋₂—C(O)—[C(R^{e}R^{f})]₁₋₂—, —[C(R^{e}R^{f})]₁₋₂—C(O)NH—[C(R^{e}R^{f})]₁₋₂—, —[C(R^{e}R^{f})]₁₋₂—NHC(O)—[C(R^{e}R^{f})]₁₋₂—, —[C(R^{e}R^{f})]₁₋₂—S(O)₂—[C(R^{e}R^{f})]₁₋₂—, —[C(R^{e}R^{f})]₁₋₂—NHS(O)₂—[C(R^{e}R^{f})]₁₋₂— or —[C(R^{e}R^{f})]₁₋₂—S(O)₂NH—[C(R^{e}R^{f})]₁₋₂—;
R^{12f} is H, F, CHF₂, CH₂F, CF₃ or CN;
each R^{e} and R^{f} is independently H, a halogen, a C₁₋₃ alkyl group, or a C₃₋₆ cycloalkyl group;
alternatively, R^{e} and R^{f} along with the carbon atom connected to them, form a cyclopropyl group;
p is 0, 1, 2, 3 or 4;
q is 0, 1, 2, 3 or 4;
v is 0, 1, 2 or 3;
u is 0, 1 or 2; and
the heteroatom in the above heteroaryl group is independently N, O or S, and the number of heteroatoms in the above heteroaryl group is independently 1, 2, 3 or 4.

2. The compound as claimed in claim 1, or a tautomer, stereoisomer, racemate or isotopic derivative thereof, or a pharmaceutically acceptable salt of any of the foregoing, or a crystalline or solvated form of any of the foregoing, which is characterized by: R^{1a} is a methyl group;
and/or, ring B is a benzene ring;
and/or, the C₂₋₆ alkyl group in the definition of R^{5a} is an ethyl group, a n-propyl group or a n-butyl group;
and/or, the halogen in the definition of R⁴ is fluorine;
and/or, the C₁₋₄ alkyl group in the definition of R⁴ is a methyl group;
and/or, the C₁₋₄ haloalkyl group in the definition of R⁴ is a C₁₋₄ fluoroalkyl group;
and/or, the —O—C₁₋₄ alkyl group in the definition of R⁴ is —O—CH₃;
and/or, the —O—C₁₋₄ haloalkyl group in the definition of R⁴ is a —O—C₁₋₄ fluoroalkyl group;
and/or, the C₁₋₄ hydroxyalkyl group in the definition of R⁴ is —CH₂OH;
and/or, “unsubstituted or substituted 6-10-membered aryl group” in the definition of R^{5a} is “unsubstituted or substituted phenyl group”, or “unsubstituted or substituted naphthyl group”;
and/or, “unsubstituted or substituted 5-10-membered heteroaryl group” in the definition of R^{5a} is “unsubstituted or substituted 5- or 6-membered heteroaryl group”;
and/or, the halogen in the definitions of R^{5g} and R^{5h} is fluorine;
and/or, the C₁₋₄ alkyl group in the definitions of R^{5g} and R^{5h} is a methyl group;
and/or, the —O—C₁₋₄ haloalkyl group in the definitions of R^{5g} and R^{5h} is —OCF₃;
and/or, the C₁₋₄ haloalkyl group in the definitions of R^{5g} and R^{5h} is a C₁₋₄ fluoroalkyl group;
and/or, the —O—C₁₋₄ alkyl group in the definitions of R^{5g} and R^{5h} is —O—CH₃, and/or, “unsubstituted or substituted 6-10-membered aryl group” in the definition of R^{5b} is “unsubstituted or substituted phenyl group”, or “unsubstituted or substituted naphthyl group”;
and/or, the halogen in the definition of R^{a} is fluorine;
and/or, the C₁₋₃ alkyl group in the definition of R^{a} is a methyl or ethyl group;
and/or, the halogen in the definition of R^{b} is fluorine;
and/or, the C₁₋₃ alkyl group in the definition of R^{b} is a methyl or ethyl group;
and/or, each L¹ is independently —[C(R^{a}R^{b})]₁₋₅- or —[C(R^{a}R^{b})]₁₋₂—C(O)NH—[C(R^{a}R^{b})]₁₋₂—;
and/or, m is 1;
and/or, n is 0, 1 or 2;
and/or, the 5-6-membered heteroaromatic ring in the definition of ring C is an imidazole, thiazole, furan, thiophene or pyridine;
and/or, the halogen in the definition of R⁶ is fluorine;
and/or, the C₁₋₄ alkyl group in the definition of R⁶ is a methyl group;
and/or, the C₁₋₄ haloalkyl group in the definition of R⁶ is a C₁₋₄ fluoroalkyl group;
and/or, the —O—C₁₋₄ alkyl group in the definition of R⁶ is —O—CH₃;
and/or, the —O—C₁₋₄ haloalkyl group in the definition of R⁶ is a —O—C₁₋₄ fluoroalkyl group,
and/or, the —S—C₁₋₄ alkyl group in the definition of R⁶ is —S—CH₃;
and/or, the —S(O)₂—C₁₋₄ alkyl group in the definition of R⁶ is —S(O)₂—CH₃;
and/or, the C₁₋₄ hydroxyalkyl group in the definition of R⁶ is —CH₂—OH;
and/or, the C₂₋₆ alkenyl group in the definitions of R^{8a}, R^{8b}, R^{8c}, R^{8d}, R^{8e} and R^{8k} is a vinyl group;
and/or, the C₃₋₆ cycloalkyl group in the definitions of R^{8a}, R^{8b}, R^{8c}, R^{8d}, R^{8e} and R^{8k} is a cyclopropyl group;
and/or, 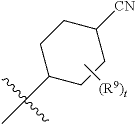
is cis or trans conformation;
and/or, t is 0;
and/or, y is 0;
and/or, z is 0, 1 or 2;
and/or, the C₃₋₆ cycloalkyl group in the definition of ring D is a cyclopropyl or cyclobutyl group;
and/or, the 5-6-membered heteroaromatic ring in the definition of ring D is a furan or a thiophene;
and/or, the 5-6-membered heteroaromatic ring in the definition of ring E is pyrazole;
and/or, the halogens in the definitions of R¹⁰ and R¹¹ are fluorine or chlorine;
and/or, the C₁₋₄ alkyl group in the definitions of R¹⁰ and R¹¹ is a methyl group;
and/or, the C₁₋₄ haloalkyl group in the definitions of R¹⁰ and R¹¹ is a C₁₋₄ fluoroalkyl group;
and/or, the —O—C₁₋₄ alkyl group in the definitions of R¹⁰ and R¹¹ is —O—CH₃;
and/or, the —O—C₁₋₄ haloalkyl group in the definitions of R¹⁰ and R¹¹ is a —O—C₁₋₄ fluoroalkyl group;
and/or, the —S—C₁₋₄ alkyl group in the definitions of R¹⁰ and R¹¹ is —S—CH₃;
and/or, the —S(O)₂—C₁₋₄alkyl group in the definitions of R¹⁰ and R¹¹ is —S(O)₂—CH₃;
and/or, the C₁₋₄ hydroxyalkyl group in the definitions of R¹⁰ and R¹¹ is —CH₂—OH;
and/or, q is 0;
and/or, p is 0, 1 or 2;
and/or, the —S(O)₂—C₁₋₄ alkyl group in the definition of R^{14b} is —S(O)₂—CH₂CH₃, —S(O)₂—CH₂CH₂CH₃ or —S(O)₂—CH₂CH₂CH₂CH₃;
and/or, the halogen in the definitions of R^{e} and R^{f} is fluorine;
and/or, the C₁₋₃ alkyl group in the definitions of R^{e} and R^{f} is a methyl or ethyl group;
and/or, the C₃₋₆ cycloalkyl group in the definitions of R^{e} and R^{f} is a cyclopropyl group, a cyclobutyl group or a cyclopentyl group.

3. The compound as claimed in claim 1, or a tautomer, stereoisomer, racemate or isotopic derivative thereof, or a pharmaceutically acceptable salt of any of the foregoing, or a crystalline or solvated form of any of the foregoing, which is characterized by: 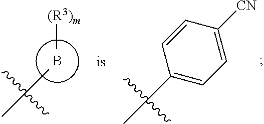
and/or, each R⁴ is independently a halogen or a C₁₋₄ hydroxyalkyl group, or, two R⁴ located at the same carbon atom or at different carbon atoms are interconnected to form —CH₂— or —(CH₂)₂—;
and/or, “unsubstituted or substituted 6-10-membered aryl group” in the definition of R^{5a} is “unsubstituted phenyl group”, or “phenyl group independently substituted with 1, 2, 3 or 4 of the following functional groups: a halogen, a C₁₋₄alkyl group, a —O—C₁₋₄haloalkyl group, or a phenyl group”;
and/or, “unsubstituted or substituted 5-10-membered heteroaryl group” in the definition of R⁵ is “unsubstituted or substituted thiophene group”;
and/or, “unsubstituted or substituted 6-10-membered aryl group” in the definition of R^{5b} is “unsubstituted phenyl group”, or “phenyl group independently substituted with 1, 2, 3 or 4 of the following functional groups: a halogen or a cyano group”;
and/or, -L¹-R^{5f} in the definition of R^{5b} is —C(R^{a}R^{b})R^{5f};
and/or, the C₁₋₄ haloalkyl group in the definitions of R^{5g} and R^{5h} is —CF₃;
and/or, each R^{a} is independently H, fluorine, a methyl group or an ethyl group; each R^{b} is independently H, fluorine, a methyl group or an ethyl group, or, R^{a} and R^{b} along with the carbon atom connected to them, form a cyclopropyl group;
and/or, when R² is 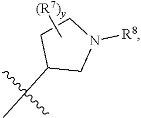
ring C is a benzene ring, imidazole, furan or pyridine;
and/or, when R² is 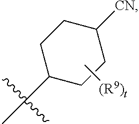
ring C is a benzene ring, thiazole, furan or thiophene;
and/or, R⁸ is R⁵, —S(O)₂R^{8a} or —C(O)R^{8b};
and/or, each R⁶ is independently fluorine, a hydroxyl group, an amino group, a cyano group, a methyl group, CF₃, —O—CH₃, —S—CH₃, —S(O)₂—CH₃ or —CH₂—OH;
and/or, R¹² is a cyano group, —C(R^{e}R^{f})—R^{12f}, —[C(R^{e}R^{f})₂]—R^{12f}, —[C(R^{e}R^{f})₃]—R^{12f} or 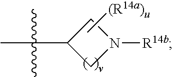
and/or, R^{14a} is a cyano-substituted C₁₋₄ alkyl group;
and/or, v is 0;
and/or, u is 1.

4. The compound as claimed in claim 1, or a tautomer, stereoisomer, racemate or isotopic derivative thereof, or a pharmaceutically acceptable salt of any of the foregoing, or a crystalline or solvated form of any of the foregoing, which is characterized by: each R^{8g} is independently a halogen, a C₁₋₄ alkyl group, a —O—C₁₋₄ haloalkyl group or a phenyl group;
and/or, “unsubstituted or substituted 6-10-membered aryl group” in the definition of R^{5a} is 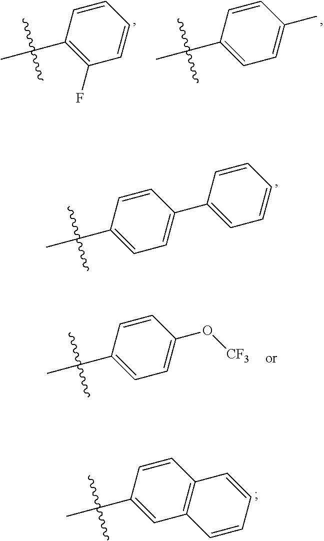
and/or, “unsubstituted or substituted 5-10-membered heteroaryl group” in the definition of R^{5a} is 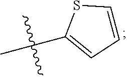 [SMILES: *c1cccs1]
and/or, “unsubstituted or substituted 6-10-membered aryl group” in the definition of R^{5b} is 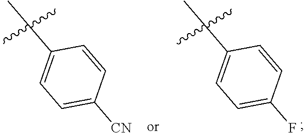 [SMILES: *c1ccc(C#N)cc1 ; *c1ccc(F)cc1]
and/or, -L¹-R^{5f} in the definition of R^{5b} is 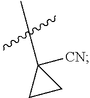 [SMILES: *C1(C#N)CC1]
and/or, the C₁₋₆ alkyl group in the definition of R^{5c} is a tert-butyl group;
and/or, -L¹-R^{5f} in the definition of R⁵ is —[C(R^{a}R^{b})]₁₋₅—R^{5f} or —[C(R^{a}R^{b})]₁₋₂—C(O)NH—[C(R^{a}R^{b})]₁₋₂;
and/or, R⁸ is —S(O)₂C(R^{a}R^{b})—C₁₋₆ alkyl, —S(O)₂—CF₃, —S(O)₂C(R^{a}R^{b})—R^{5f}, —S(O)₂[C(R^{a}R^{b})₂]—R^{5f}, —S(O)₂[C(R^{a}R^{b})₃]—R^{5f}, —C(R^{a}R^{b})—R^{5f}, —[C(R^{a}R^{b})₂]—R^{5f}, —[C(R^{a}R^{b})₃]—R^{5f} 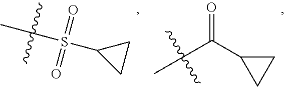 [SMILES: *S(=O)(=O)C1CC1 ; *C(=O)C1CC1]
—C(O)CH═CH₂ or —C(O)NHC(R^{a}R^{b})—R^{5f};
and/or, 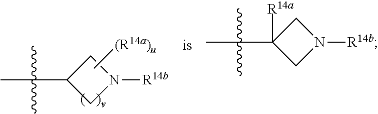
and/or, R^{14a} is —CH₂CN;
and/or, R^{14b} is a —S(O)₂—C₁₋₄ alkyl group;

5. The compound as claimed in claim 1, or a tautomer, stereoisomer, racemate or isotopic derivative thereof, or a pharmaceutically acceptable salt of any of the foregoing, or a crystalline or solvated form of any of the foregoing, which is characterized by: R^{5a} is a C₂₋₆ alkyl group, an unsubstituted or substituted 6-10-membered aryl group or an unsubstituted or substituted 5-10-membered heteroaryl group;
and/or, R^{5b} is -L¹-R^{5f}, or an unsubstituted or substituted phenyl group;
and/or, R^{5c} is a C₁₋₆ alkyl group;
and/or, R^{5c}, R^{5d} and R^{5k} are each independently -L¹-R^{5f};
and/or, each R^{5f} is independently F, CF₃ or CN;
and/or, R⁸ is —S(O)₂(CH₂)₂—CH₃, —S(O)₂—CF₃, —C(O)NHCH₂—CF₃, —C(O)CH═CH₂, 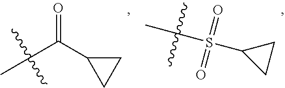 [SMILES: *C(=O)C1CC1 ; *S(=O)(=O)C1CC1]
—CH₂—CN, —(CH₂)₂—CN or —(CH₂)₃—CF₃;
and/or, 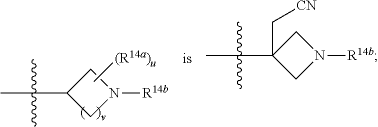
and/or, R^{14b} is a —S(O)₂—C₁₋₄ alkyl group;
and/or 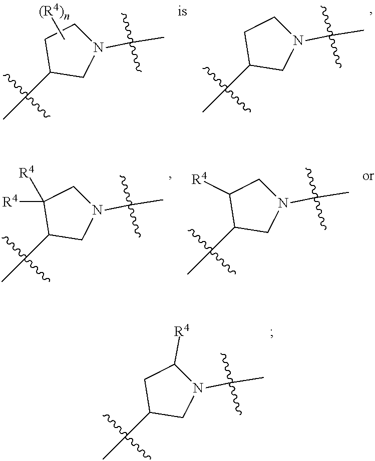
and/or, ring C is a benzene ring, or

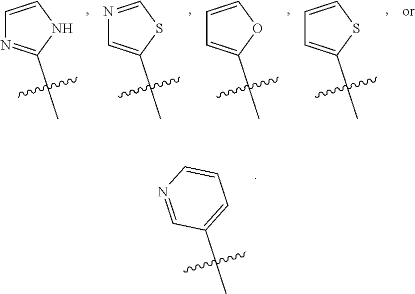 [SMILES: *c1ncc[nH]1 ; *c1cncs1 ; *c1ccco1 ; *c1cccs1 ; *c1cccnc1]

6. The compound as claimed in claim 1, or a tautomer, stereoisomer, racemate or isotopic derivative thereof, or a pharmaceutically acceptable salt of any of the foregoing, or a crystalline or solvated form of any of the foregoing, which is characterized by: —C(O)NHR^{5k} in the definition of R⁵ is —C(O)NHCH₂—CF₃;
and/or, —S(O)₂R^{a} in the definition of R⁵ is —S(O)₂(CH₂)₂—CF₃, —S(O)₂(CH₂)₂—CH, —S(O)₂CH₂—CH₃, —S(O)₂(CH₂)₃—CH₃, —S(O)₂(CH₂)₂—CF₃, 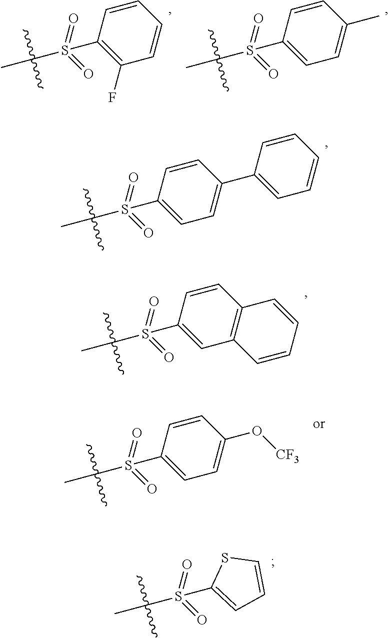 [SMILES: *S(=O)(=O)c1ccccc1F ; *S(=O)(=O)c1ccc(C)cc1 ; *S(=O)(=O)c1ccc(-c2ccccc2)cc1 ; *S(=O)(=O)c1ccc2ccccc2c1 ; *S(=O)(=O)c1ccc(OC(F)(F)F)cc1 ; *S(=O)(=O)c1cccs1]
and/or, —C(O)R^{5b} in the definition of R⁵ is 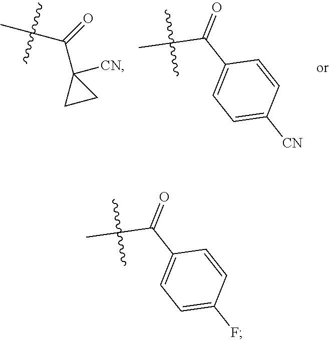 [SMILES: *C(=O)C1(C#N)CC1 ; *C(=O)c1ccc(C#N)cc1 ; *C(=O)c1ccc(F)cc1]
and/or, —C(O)OR^{5e} in the definition of R⁵ is —C(O)OC(CH₃)₂—CH₃;
and/or, -L¹-R^{5f} in the definition of R⁵ is —C(R^{a}R^{b})—CN, —[C(R^{a}R^{b})]₂—CN, —[C(R^{a}R^{b})]₃—CN, —[C(R^{a}R^{b})]₄—CN, —[C(R^{a}R^{b})]—CF₃, —[C(R^{a}R^{b})]₂—CF₃, —[C(R^{a}R^{b})]₃—CF₃, —C(R^{a}R^{b})C(O)NH—C(R^{a}R^{b})CF₃ or —[C(R^{a}R^{b})]₅—F.

7. The compound as claimed in claim 1, or a tautomer, stereoisomer, racemate or isotopic derivative thereof, or a pharmaceutically acceptable salt of any of the foregoing, or a crystalline or solvated form of any of the foregoing, which is characterized by: R⁵ is —CH₂—CN, —(CH₂)₂—CN, —(CH₂)₃—CN, —(CH₂)₄—CN, —(CH₂)₂—CF₃, —CH₂(CF₂)₂—CF₃, —(CF₂)₃—CF₃, —(CH₂)₂CF₂—CF₃, —(CH₂)₃—CF₃, —CH(CH₂CH₃)C(O)NH—CH₂CF₃, —CH₂C(O)NHCH₂—CF₃, —CH(CH₃)C(O)NHCH₂—CF₃, —(CH₂)₅—F, 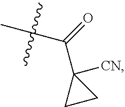 [SMILES: *C(=O)C1(C#N)CC1]
—S(O)₂(CH₂)₂—CF₃, —C(O)NHCH₂—CF₃, —S(O)₂(CH₂)₂—CH₃, —S(O)₂CH₂—CH₃, —S(O)₂(CH₂)₃—CH, —S(O)₂(CH₂)₂—CF₃, 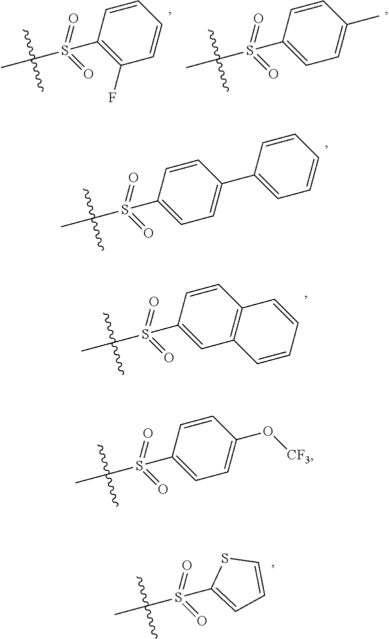 [SMILES: *S(=O)(=O)c1ccccc1F ; *S(=O)(=O)c1ccc(C)cc1 ; *S(=O)(=O)c1ccc(-c2ccccc2)cc1 ; *S(=O)(=O)c1ccc2ccccc2c1 ; *S(=O)(=O)c1ccc(OC(F)(F)F)cc1 ; *S(=O)(=O)c1cccs1]
C(O)OC(CH₃)₂—CH₃; 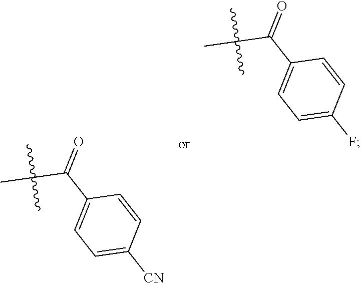 [SMILES: *C(=O)c1ccc(F)cc1 ; *C(=O)c1ccc(C#N)cc1]
and/or, 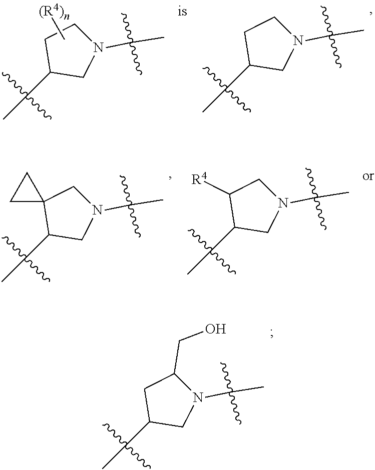
and/or, 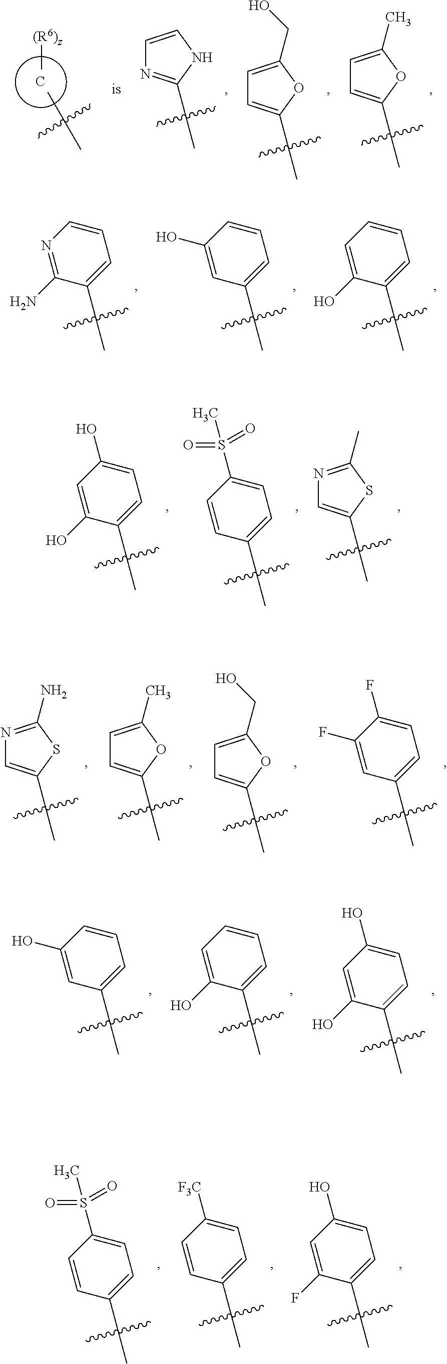 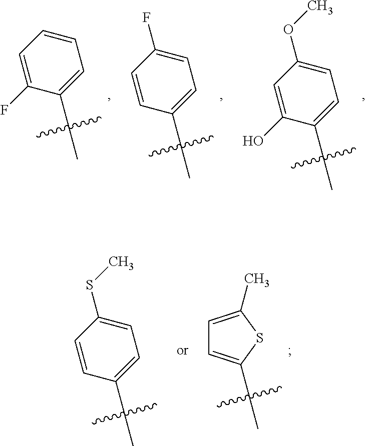
and/or R⁸ is —S(O)₂C(R^{a}R^{b})—CH₃, —S(O)₂—CF₃, —C(O)NHC(R^{a}R^{b})—CF₃, —C(O)CH═CH₂, 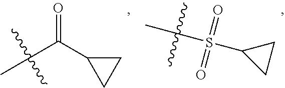 [SMILES: *C(=O)C1CC1 ; *S(=O)(=O)C1CC1]
—C(R^{a}R^{b})—CN, —[C(R^{a}R^{b})₂]CN, —[C(R^{a}R^{b})₃]CF₃;
and/or 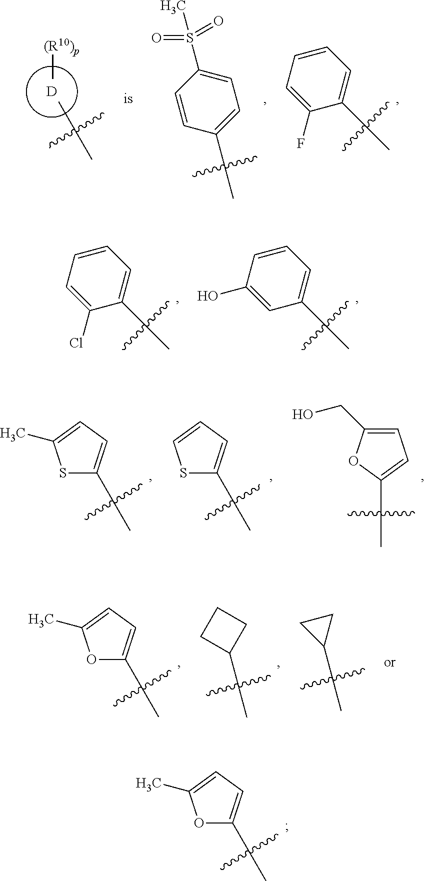
and/or, R¹² is —CN, —CH₂—CN, —(CH₂)₂—CN, —(CH₂)₃—CN,

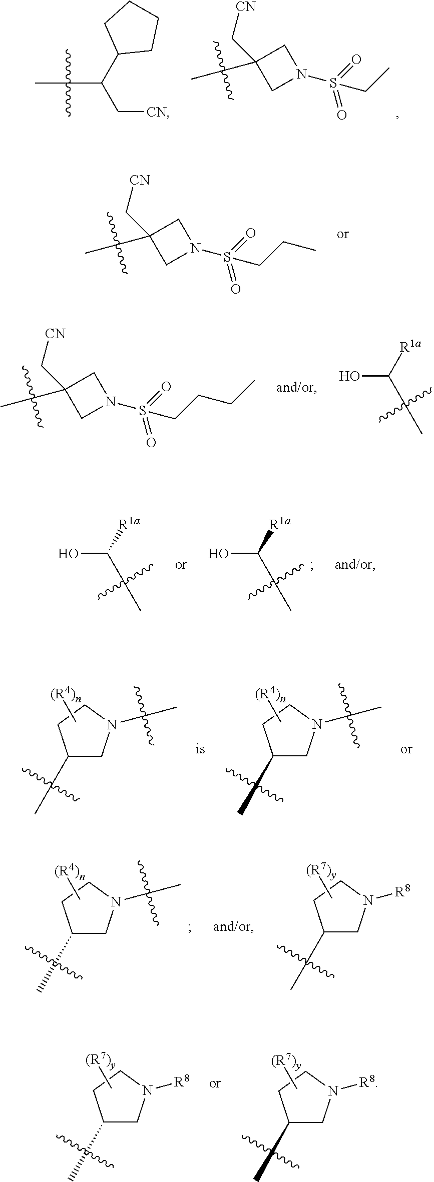

8. The compound as claimed in claim 1, or a tautomer, stereoisomer, racemate or isotopic derivative thereof, or a pharmaceutically acceptable salt of any of the foregoing, or a crystalline or solvated form of any of the foregoing, which is characterized by:
said compound has the following formulae I-1a, I-1b, I-1c, I-1d, I-2, I-3C, I-3D, I-3E, I-3F, I-3G, I-4E, I-4F, I-4J, 1-4L, 1-4M, I-5, I-6, I-7A, I-7B, I-7C, I-7D, I-7E, I-7F, I-7G or the structure shown in I-7H; 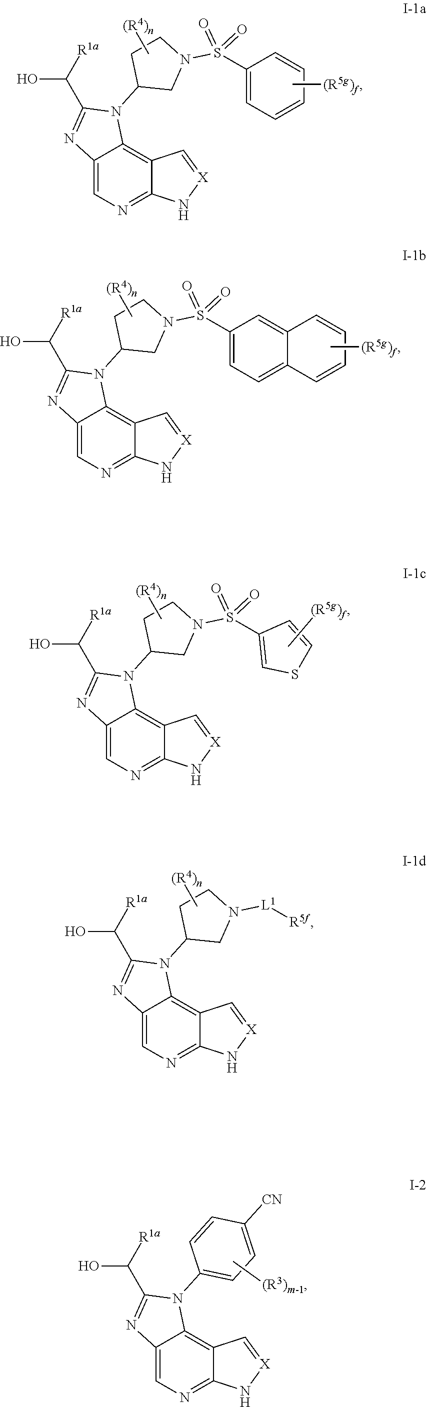 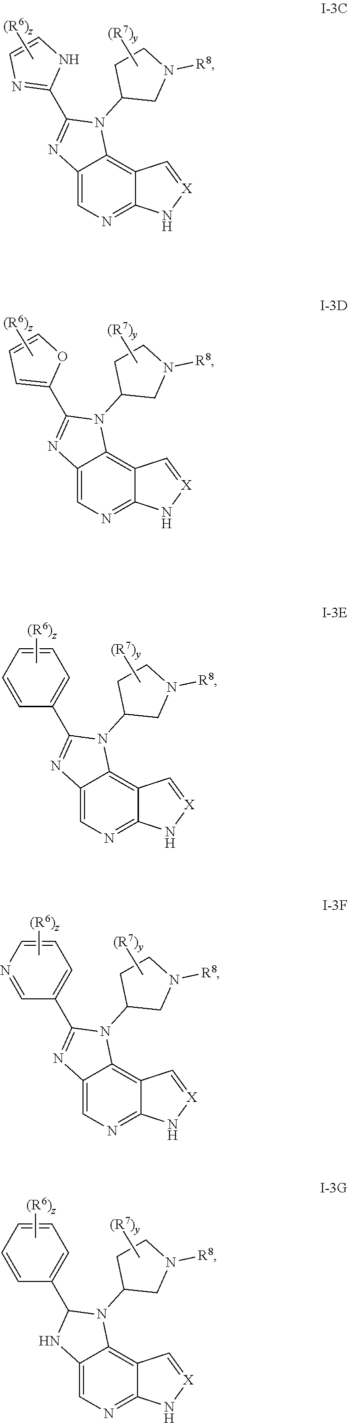 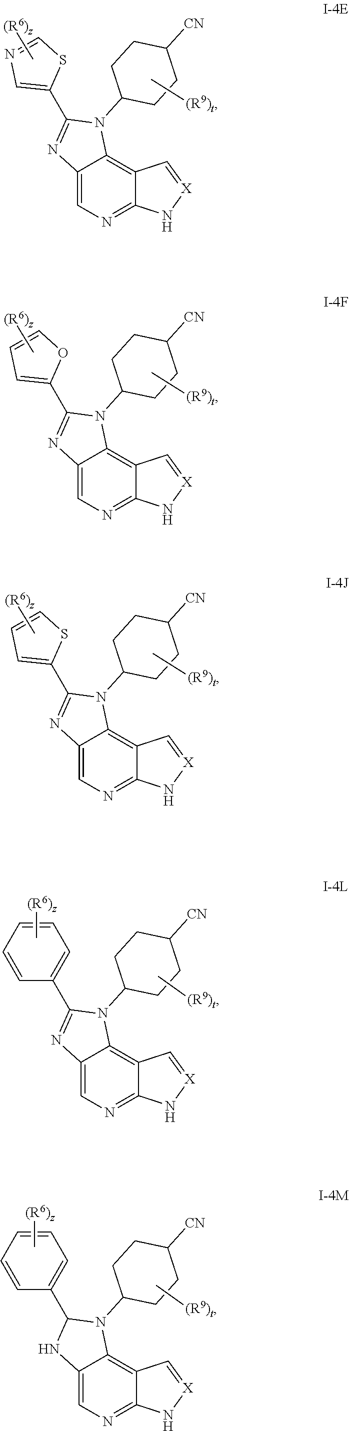 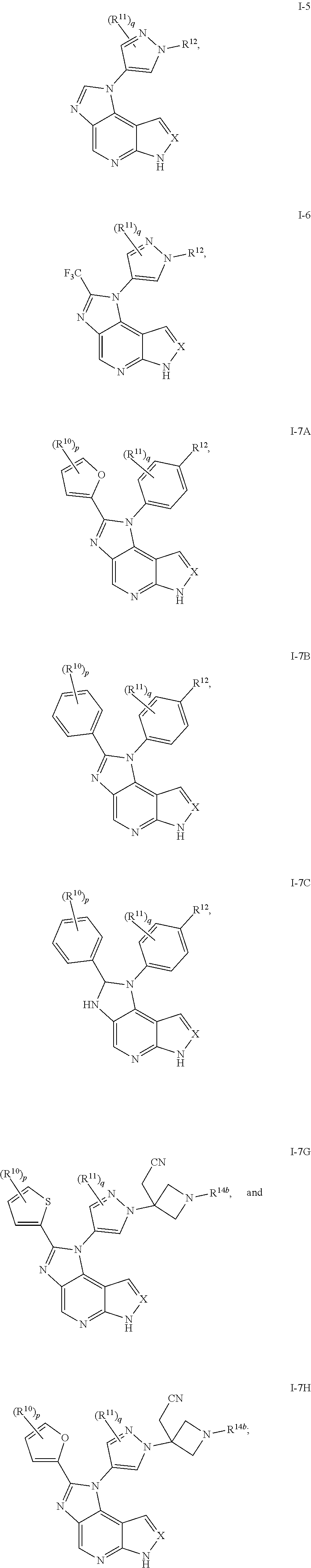
each f is independently 0, 1, 2, 3 or 4.

9. The compound as claimed in claim 1, or a tautomer, stereoisomer, racemate or isotopic derivative thereof, or a pharmaceutically acceptable salt of any of the foregoing, or a crystalline or solvated form of any of the foregoing, characterized by that said compound has any of the following structures:

| Chemical name | Structure |
|---|---|
| Tert-butyl 3-(2-((R)-1- hydroxyethyl)imidazo[4,5- d]pyrrolo[2,3-b]pyridin-1(6H)- yl)pyrrolidine-1-carboxylate | 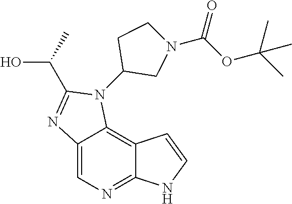 [SMILES: C[C@@H](O)c1nc2cnc3[nH]ccc3c2n1C1CCN(C(=O)OC(C)(C)C)C1] |
| 4-(3-(2-((R)-1- Hydroxyethyl)imidazo[4,5- d]pyrrolo[2,3-b]pyridin-1(6H)- yl)pyrrolidin-1-carbonyl)benzonitrile | 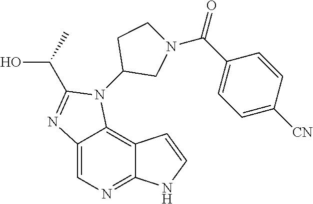 [SMILES: C[C@@H](O)c1nc2cnc3[nH]ccc3c2n1C1CCN(C(=O)c2ccc(C#N)cc2)C1] |
| (4-Fluorophenyl)(3-(2-((R)-1- Hydroxyethyl)imidazo[4,5- d]pyrrolo[2,3-b]pyridin-1(6H)- yl)pyrrolidin-1-yl)methanone | 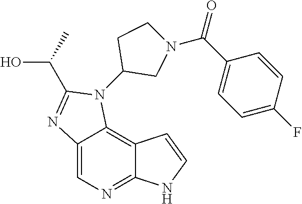 [SMILES: C[C@@H](O)c1nc2cnc3[nH]ccc3c2n1C1CCN(C(=O)c2ccc(F)cc2)C1] |
| (1R)-1-(1-(1-(ethylsulfonyl)pyrrolidin- 3-yl)-1,6-dihydroimidazo[4,5- d]pyrrolo[2,3-b]pyridin-2-yl)ethan-1-ol | 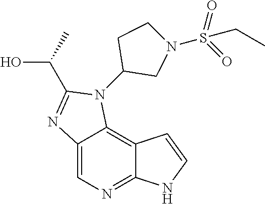 [SMILES: CCS(=O)(=O)N1CCC(n2c([C@@H](C)O)nc3cnc4[nH]ccc4c32)C1] |
| (1R)-1-(1-(1-(propylsulfonyl)pyrrolidin- 3-yl)-1,6-dihydroimidazo[4,5- d]pyrrolo[2,3-b]pyridin-2-yl)ethan-1-ol | 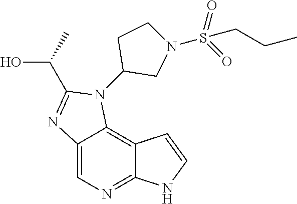 [SMILES: CCCS(=O)(=O)N1CCC(n2c([C@@H](C)O)nc3cnc4[nH]ccc4c32)C1] |
| (1R)-1-(1-(1-(butylsulfonyl)pyrrolidin- 3-yl)-1,6-dihydroimidazo[4,5- d]pyrrolo[2,3-b]pyridin-2-yl)ethan-1-ol | 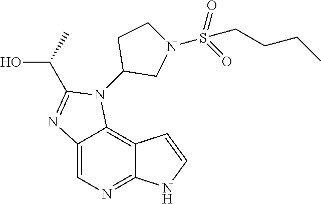 [SMILES: CCCCS(=O)(=O)N1CCC(n2c([C@@H](C)O)nc3cnc4[nH]ccc4c32)C1] |
| (1R)-1-(1-(1-((2- fluorophenyl)sulfonyl)pyrrolidin-3-yl)- 1,6-dihydroimidazo[4,5-d]pyrrolo[2,3- b]pyridin-2-yl)ethan-1-ol | 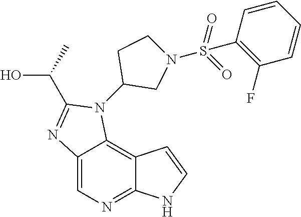 [SMILES: C[C@@H](O)c1nc2cnc3[nH]ccc3c2n1C1CCN(S(=O)(=O)c2ccccc2F)C1] |
| (1R)-1-(1-(1-tosylpyrrolidin-3-yl)-1,6- dihydroimidazo[4,5-d]pyrrolo[2,3- b]pyridin-2-yl)ethan-1-ol | 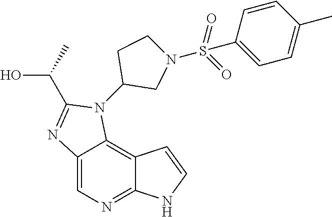 [SMILES: Cc1ccc(S(=O)(=O)N2CCC(n3c([C@@H](C)O)nc4cnc5[nH]ccc5c43)C2)cc1] |
| (1R)-1-(1-(1-([1,1′-biphenyl]-4- ylsulfonyl)pyrrolidin-3-yl)-1,6- dihydroimidazo[4,5-d]pyrrolo[2,3- b]pyridin-2-yl)ethan-1-ol | 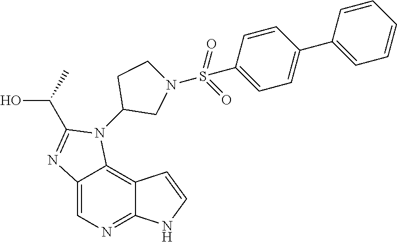 [SMILES: C[C@@H](O)c1nc2cnc3[nH]ccc3c2n1C1CCN(S(=O)(=O)c2ccc(-c3ccccc3)cc2)C1] |
| (1R)-1-(1-(1-(naphthalen-2- ylsulfonyl)pyrrolidin-3-yl)-1,6- dihydroimidazo[4,5-d]pyrrolo[2,3- b]pyridin-2-yl)ethan-1-ol | 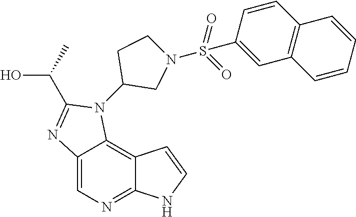 [SMILES: C[C@@H](O)c1nc2cnc3[nH]ccc3c2n1C1CCN(S(=O)(=O)c2ccc3ccccc3c2)C1] |
| (1R)-1-(1-(1-((4- (trifluoromethoxy)phenyl)sulfonyl) pyrrolidin-3-yl)-1,6-dihydroimidazo[4,5- d]pyrrolo[2,3-b]pyridin-2-yl)ethan-1-ol | 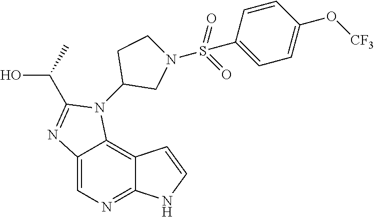 [SMILES: C[C@@H](O)c1nc2cnc3[nH]ccc3c2n1C1CCN(S(=O)(=O)c2ccc(OC(F)(F)F)cc2)C1] |
| (1R)-1-(1-(1-(thiophen-2- ylsulfonyl)pyrrolidin-3-yl)-1,6- dihydroimidazo[4,5-d]pyrrolo[2,3- b]pyridin-2-yl)ethan-1-ol | 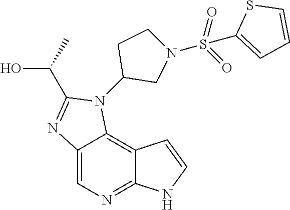 [SMILES: C[C@@H](O)c1nc2cnc3[nH]ccc3c2n1C1CCN(S(=O)(=O)c2cccs2)C1] |
| 2-(1-(Ethylsulfonyl)-3-(4-(2-(5- (hydroxymethyl)furan-2- yl)imidazo[4,5-d]pyrrolo[2,3-b]pyridin- 1(6H)-yl)-1H-pyrazol-1-yl)azetidin-3- yl)acetonitrile | 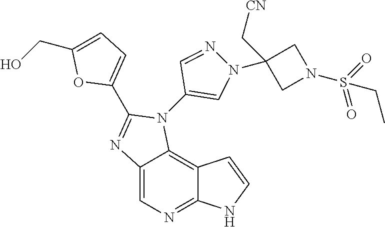 [SMILES: CCS(=O)(=O)N1CC(CC#N)(n2cc(-n3c(-c4ccc(CO)o4)nc4cnc5[nH]ccc5c43)cn2)C1] |
| 2-(1-(Propylsulfonyl)-3-(4-(2-(5- (hydroxymethyl)furan-2- yl)imidazo[4,5-d]pyrrolo[2,3-b]pyridin- 1(6H)-yl)-1H-pyrazol-1-yl)azetidin-3- yl)acetonitrile | 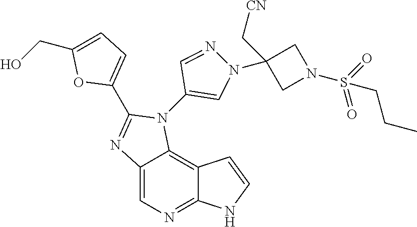 [SMILES: CCCS(=O)(=O)N1CC(CC#N)(n2cc(-n3c(-c4ccc(CO)o4)nc4cnc5[nH]ccc5c43)cn2)C1] |
| 2-(1-(Butylsulfonyl)-3-(4-(2-(5- (hydroxymethyl)furan-2- yl)imidazo[4,5-d]pyrrolo[2,3-b]pyridin- 1(6H)-yl)-1H-pyrazol-1-yl)azetidin-3- yl)acetonitrile | 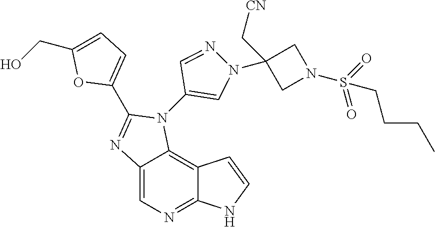 [SMILES: CCCCS(=O)(=O)N1CC(CC#N)(n2cc(-n3c(-c4ccc(CO)o4)nc4cnc5[nH]ccc5c43)cn2)C1] |
| (5-(1-(1-(thiophen-2- ylsulfonyl)pyrrolidin-3-yl)-1,6- dihydroimidazo[4,5-d]pyrrolo[2,3- b]pyridin-2-yl)furan-2-yl)methanol | 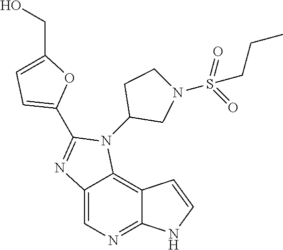 [SMILES: CCCS(=O)(=O)N1CCC(n2c(-c3ccc(CO)o3)nc3cnc4[nH]ccc4c32)C1] |
| Cyclopropyl(3-(2-(5- (hydroxymethyl)furan-2- yl)imidazo[4,5-d]pyrrolo[2,3-b]pyridin- 1(6H)-yl)pyrrolidin-1-yl)methanone | 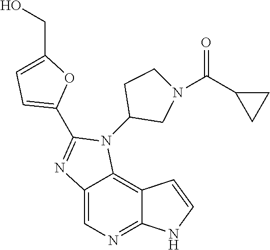 [SMILES: O=C(C1CC1)N1CCC(n2c(-c3ccc(CO)o3)nc3cnc4[nH]ccc4c32)C1] |
| 2-(1H-imidazol-2-yl)-1-(1- (propylsulfonyl)pyrrolidin-3-yl)-1,6- dihydroimidazo[4,5-d]pyrrolo[2,3- b]pyridine | 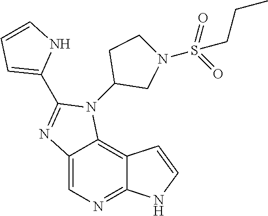 [SMILES: CCCS(=O)(=O)N1CCC(n2c(-c3ccc[nH]3)nc3cnc4[nH]ccc4c32)C1] |
| 2-(1H-imidazol-2-yl)-1-(1- ((trifluoromethyl)sulfonyl)pyrrolidin-3- yl)-1,6-dihydroimidazo[4,5- d]pyrrolo[2,3-b]pyridine | 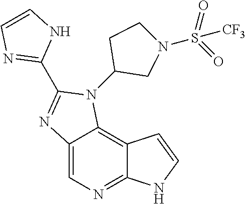 [SMILES: O=S(=O)(N1CCC(n2c(-c3ncc[nH]3)nc3cnc4[nH]ccc4c32)C1)C(F)(F)F] |
| (R)-3-(2-((R)-1- Hydroxyethyl)imidazo[4,5- d]pyrrolo[2,3-b]pyridin-1(6H)-yl)-N- (2,2,2-trifluoroethyl)pyrrolidine-1- carboxamide | 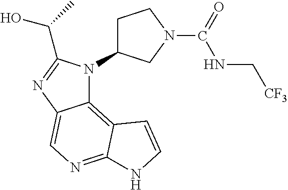 [SMILES: C[C@@H](O)c1nc2cnc3[nH]ccc3c2n1[C@H]1CCN(C(=O)NCC(F)(F)F)C1] |
| (R)-1-(1-((R)-1- (Propylsulfonyl)pyrrolidin-3-yl)-1,6- dihydroimidazo[4,5-d]pyrrolo[2,3- b]pyridin-2-yl)ethanol | 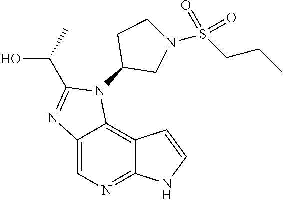 [SMILES: CCCS(=O)(=O)N1CC[C@H](n2c([C@@H](C)O)nc3cnc4[nH]ccc4c32)C1] |
| trans-4-(2-(5-(Hydroxymethyl)furan-2- yl)imidazo[4,5-d]pyrrolo[2,3-b]pyridin- 1(6H)-yl)cyclohexanecarbonitrile | 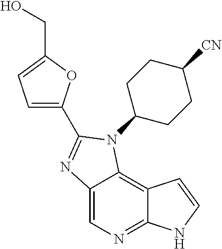 [SMILES: N#C[C@H]1CC[C@@H](n2c(-c3ccc(CO)o3)nc3cnc4[nH]ccc4c32)CC1] |
| (R)-3-(2-(5-(Hydroxymethyl)furan-2- yl)imidazo[4,5-d]pyrrolo[2,3-b]pyridin- 1(6H)-yl)-N-(2,2,2- trifluoroethyl)pyrrolidine-1- carboxamide | 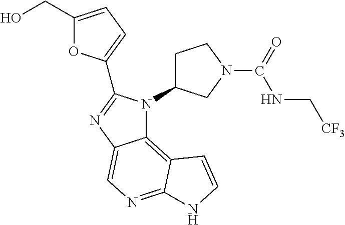 [SMILES: O=C(NCC(F)(F)F)N1CC[C@H](n2c(-c3ccc(CO)o3)nc3cnc4[nH]ccc4c32)C1] |
| (R)-3-(2-(1H-imidazol-2- yl)imidazo[4,5-d]pyrrolo[2,3-b]pyridin- 1(6H)-yl)-N-(2,2,2- trifluoroethyl)pyrrolidine-1- carboxamide | 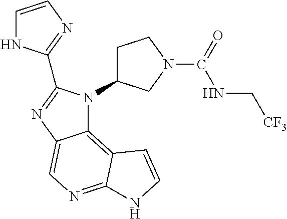 [SMILES: O=C(NCC(F)(F)F)N1CC[C@H](n2c(-c3ncc[nH]3)nc3cnc4[nH]ccc4c32)C1] |
| (R)-1-(3-(2-(1H-imidazol-2- yl)imidazo[4,5-d]pyrrolo[2,3-b]pyridin- 1(6H)-yl)pyrrolidin-1-yl)prop-2-en-1- one | 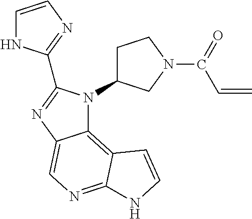 [SMILES: C=CC(=O)N1CC[C@H](n2c(-c3ncc[nH]3)nc3cnc4[nH]ccc4c32)C1] |
| trans-4-(2-(2-Methylthiazol-5- yl)imidazo[4,5-d]pyrrolo[2,3-b]pyridin- 1(6H)-yl)cyclohexanecarbonitrile | 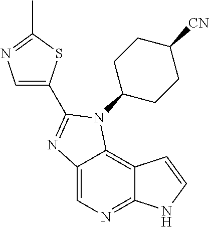 [SMILES: Cc1ncc(-c2nc3cnc4[nH]ccc4c3n2[C@H]2CC[C@@H](C#N)CC2)s1] |
| trans-4-(2-(2-Aminothiazol-5- yl)imidazo[4,5-d]pyrrolo[2,3-b]pyridin- 1(6H)-yl)cyclohexanecarbonitrile | 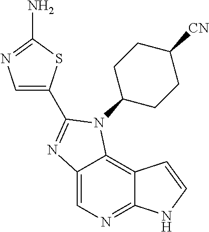 [SMILES: N#C[C@H]1CC[C@@H](n2c(-c3cnc(N)s3)nc3cnc4[nH]ccc4c32)CC1] |
| trans-4-(2-(2-Methylthiophen-5- yl)imidazo[4,5-d]pyrrolo[2,3-b]pyridin- 1(6H)-yl)cyclohexanecarbonitrile | 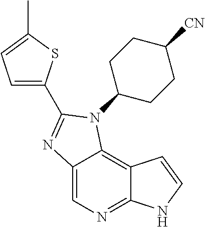 [SMILES: Cc1ccc(-c2nc3cnc4[nH]ccc4c3n2[C@H]2CC[C@@H](C#N)CC2)s1] |
| trans-4-(2-(2-Methylfuran-5- yl)imidazo[4,5-d]pyrrolo[2,3-b]pyridin- 1(6H)-yl)cyclohexanecarbonitrile | 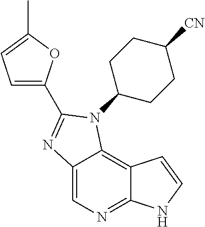 [SMILES: Cc1ccc(-c2nc3cnc4[nH]ccc4c3n2[C@H]2CC[C@@H](C#N)CC2)o1] |
| 2-(1-(1-(propylsulfonyl)pyrrolidin-3- yl)-1,6-dihydroimidazo[4,5- d]pyrrolo[2,3-b]pyridin-2-yl)phenol | 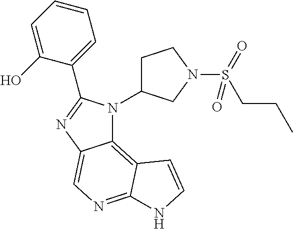 [SMILES: CCCS(=O)(=O)N1CCC(n2c(-c3ccccc3O)nc3cnc4[nH]ccc4c32)C1] |
| 4-(1-(1-(Propylsulfonyl)pyrrolidin-3- yl)-1,6-dihydroimidazo[4,5- d]pyrrolo[2,3-b]pyridin-2-yl)benzene- 1,3-diol | 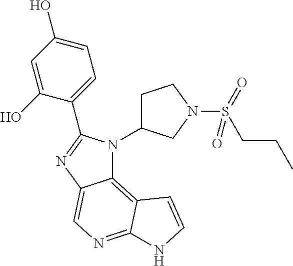 [SMILES: CCCS(=O)(=O)N1CCC(n2c(-c3ccc(O)cc3O)nc3cnc4[nH]ccc4c32)C1] |
| 3-(1-(1-(propylsulfonyl)pyrrolidin-3- yl)-1,6-dihydroimidazo[4,5- d]pyrrolo[2,3-b]pyridin-2-yl)phenol | 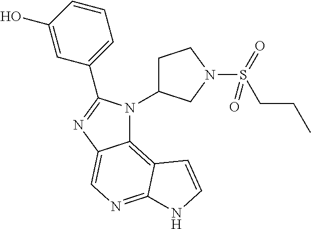 [SMILES: CCCS(=O)(=O)N1CCC(n2c(-c3cccc(O)c3)nc3cnc4[nH]ccc4c32)C1] |
| 1-(3-(2-(2,4- Dihydroxyphenyl)imidazo[4,5- d]pyrrolo[2,3-b]pyridin-1(6H)- yl)pyrrolidin-1-yl)prop-2-en-1-one | 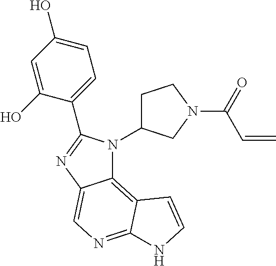 [SMILES: C=CC(=O)N1CCC(n2c(-c3ccc(O)cc3O)nc3cnc4[nH]ccc4c32)C1] |
| 3-(2-(3-Hydroxyphenyl)imidazo[4,5- d]pyrrolo[2,3-b]pyridin-1(6H)-yl)-N- (2,2,2-trifluoroethyl)pyrrolidine-1- carboxamide | 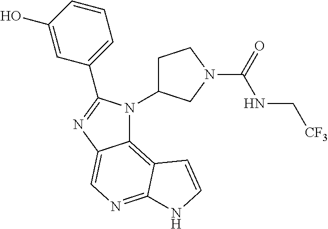 [SMILES: O=C(NCC(F)(F)F)N1CCC(n2c(-c3cccc(O)c3)nc3cnc4[nH]ccc4c32)C1] |
| trans-4-(2-(3- Hydroxyphenyl)imidazo[4,5- d]pyrrolo[2,3-b]pyridin-1(6H)- yl)cyclohexanecarbonitrile | 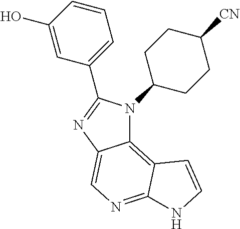 [SMILES: N#C[C@H]1CC[C@@H](n2c(-c3cccc(O)c3)nc3cnc4[nH]ccc4c32)CC1] |
| trans-4-(2-(2,4- Dihydroxyphenyl)imidazo[4,5- d]pyrrolo[2,3-b]pyridin-1(6H)- yl)cyclohexanecarbonitrile | 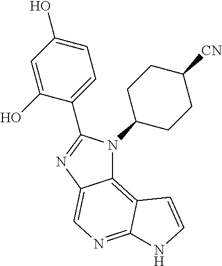 [SMILES: N#C[C@H]1CC[C@@H](n2c(-c3ccc(O)cc3O)nc3cnc4[nH]ccc4c32)CC1] |
| trans-4-(2-(4- (Trifluoromethyl)phenyl)imidazo[4,5- d]pyrrolo[2,3-b]pyridin-1(6H)- yl)cyclohexanecarbonitrile | 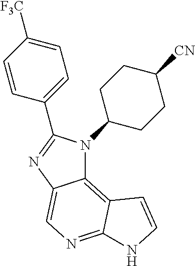 [SMILES: N#C[C@H]1CC[C@@H](n2c(-c3ccc(C(F)(F)F)cc3)nc3cnc4[nH]ccc4c32)CC1] |
| trans-4-(2-(2- Hydroxyphenyl)imidazo[4,5- d]pyrrolo[2,3-b]pyridin-1(6H)- yl)cyclohexanecarbonitrile | 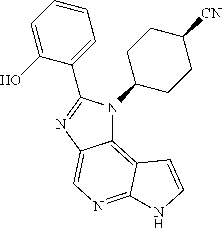 [SMILES: N#C[C@H]1CC[C@@H](n2c(-c3ccccc3O)nc3cnc4[nH]ccc4c32)CC1] |
| trans-4-(2-(2-Fluoro-4- hydroxyphenyl)imidazo[4,5- d]pyrrolo[2,3-b]pyridin-1(6H)- yl)cyclohexanecarbonitrile | 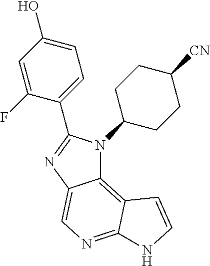 [SMILES: N#C[C@H]1CC[C@@H](n2c(-c3ccc(O)cc3F)nc3cnc4[nH]ccc4c32)CC1] |
| trans-4-(2-(2- Fluorophenyl)imidazo[4,5- d]pyrrolo[2,3-b]pyridin-1(6H)- yl)cyclohexanecarbonitrile | 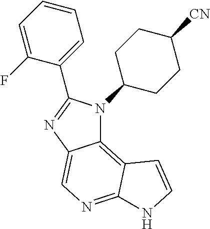 [SMILES: N#C[C@H]1CC[C@@H](n2c(-c3ccccc3F)nc3cnc4[nH]ccc4c32)CC1] |
| trans-4-(2-(2-Hydroxy-4- methoxyphenyl)imidazo[4,5- d]pyrrolo[2,3-b]pyridin-1(6H)- yl)cyclohexanecarbonitrile | 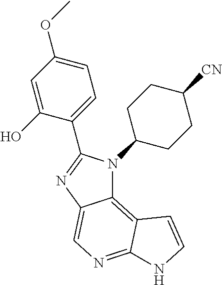 [SMILES: COc1ccc(-c2nc3cnc4[nH]ccc4c3n2[C@H]2CC[C@@H](C#N)CC2)c(O)c1] |
| trans-4-(2-(4- (Methylthio)phenyl)imidazo[4,5- d]pyrrolo[2,3-b]pyridin-1(6H)- yl)cyclohexanecarbonitrile | 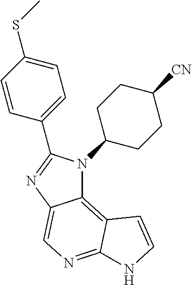 [SMILES: CSc1ccc(-c2nc3cnc4[nH]ccc4c3n2[C@H]2CC[C@@H](C#N)CC2)cc1] |
| trans-4-(2-(4- Fluorophenyl)imidazo[4,5- d]pyrrolo[2,3-b]pyridin-1(6H)- yl)cyclohexanecarbonitrile | 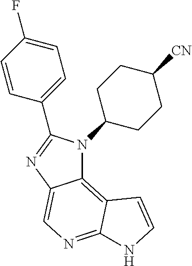 [SMILES: N#C[C@H]1CC[C@@H](n2c(-c3ccc(F)cc3)nc3cnc4[nH]ccc4c32)CC1] |
| trans-4-(2-(4-(Methylsulfonyl)phenyl)- 2,3-dihydroimidazo[4,5-d]pyrrolo[2,3- b]pyridin-1(6H)- yl)cyclohexanecarbonitrile | 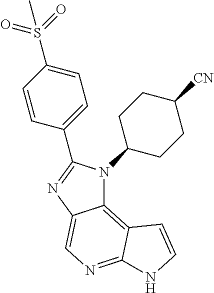 [SMILES: CS(=O)(=O)c1ccc(-c2nc3cnc4[nH]ccc4c3n2[C@H]2CC[C@@H](C#N)CC2)cc1] |
| (5-(1-(1- (cyclopropylsulfonyl)pyrrolidin-3-yl)- 1,6-dihydroimidazo[4,5-d]pyrrolo[2,3- b]pyridin-2-yl)furan-2-yl)methanol | 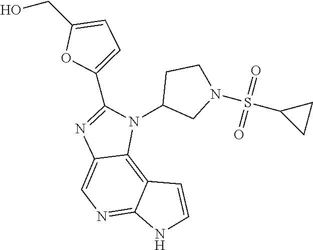 [SMILES: O=S(=O)(C1CC1)N1CCC(n2c(-c3ccc(CO)o3)nc3cnc4[nH]ccc4c32)C1] |
| 3-(4-(2-(5-(Hydroxymethyl)furan-2- yl)imidazo[4,5-d]pyrrolo[2,3-b]pyridin- 1(6H)-yl)-1H-pyrazol-1- yl)propanenitrile | 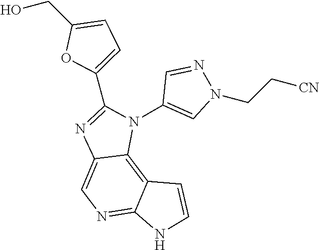 [SMILES: N#CCCn1cc(-n2c(-c3ccc(CO)o3)nc3cnc4[nH]ccc4c32)cn1] |
| 3-(1-(1-(propylsulfonyl)pyrrolidin-3- yl)-1,6-dihydroimidazo[4,5- d]pyrrolo[2,3-b]pyridin-2-yl)pyridin-2- amine | 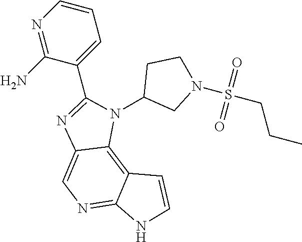 [SMILES: CCCS(=O)(=O)N1CCC(n2c(-c3cccnc3N)nc3cnc4[nH]ccc4c32)C1] |
| 2-(3-(2-((R)-1- Hydroxyethyl)imidazo[4,5- d]pyrrolo[2,3-b]pyridin-1(6H)- yl)pyrrolidin-1-yl)acetonitrile | 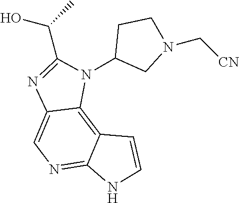 [SMILES: C[C@@H](O)c1nc2cnc3[nH]ccc3c2n1C1CCN(CC#N)C1] |
| 3-(3-(2-((R)-1- Hydroxyethyl)imidazo[4,5- d]pyrrolo[2,3-b]pyridin-1(6H)- yl)pyrrolidin-1-yl)propanenitrile | 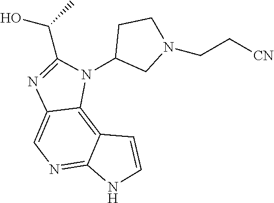 [SMILES: C[C@@H](O)c1nc2cnc3[nH]ccc3c2n1C1CCN(CCC#N)C1] |
| 3-(4-(2-(5-methylfuran-2- yl)imidazo[4,5-d]pyrrolo[2,3-b]pyridin- 1(6H)-yl)-1H-pyrazol-1- yl)propanenitrile | 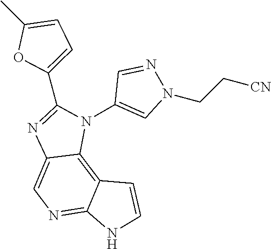 [SMILES: Cc1ccc(-c2nc3cnc4[nH]ccc4c3n2-c2cnn(CCC#N)c2)o1] |
| 3-(3-(2-(5-(Hydroxymethyl)furan-2- yl)imidazo[4,5-d]pyrrolo[2,3-b]pyridin- 1(6H)-yl)pyrrolidin-1-yl)propanenitrile | 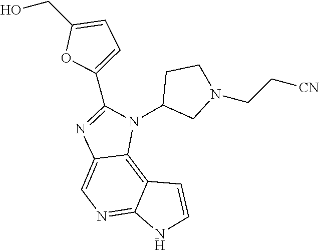 [SMILES: N#CCCN1CCC(n2c(-c3ccc(CO)o3)nc3cnc4[nH]ccc4c32)C1] |
| 2-(3-(2-(5-(hydroxymethyl)furan-2- yl)imidazo[4,5-d]pyrrolo[2,3-b]pyridin- 1(6H)-yl)pyrrolidin-1-yl)acetonitrile | 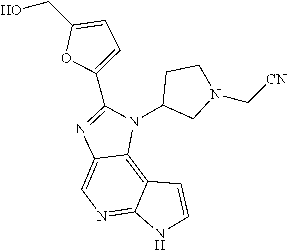 [SMILES: N#CCN1CCC(n2c(-c3ccc(CO)o3)nc3cnc4[nH]ccc4c32)C1] |
| 2-(3-(2-(3-Hydroxyphenyl)imidazo[4,5- d]pyrrolo[2,3-b]pyridin-1(6H)- yl)pyrrolidin-1-yl)acetonitrile | 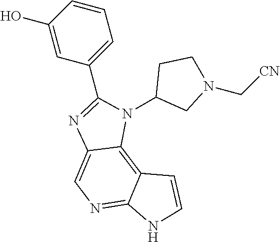 [SMILES: N#CCN1CCC(n2c(-c3cccc(O)c3)nc3cnc4[nH]ccc4c32)C1] |
| (R)-4-(2-(1-Hydroxyethyl)imidazo[4,5- d]pyrrolo[2,3-b]pyridin-1(6H)- yl)benzonitrile | 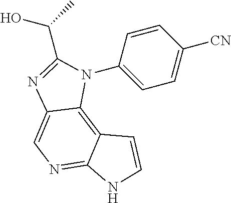 [SMILES: C[C@@H](O)c1nc2cnc3[nH]ccc3c2n1-c1ccc(C#N)cc1] |
| 4-(2-(S-(Hydroxymethyl)furan-2- yl)imidazo[4,5-d]pyrrolo[2,3-b]pyridin- 1(6H)-yl)benzonitrile | 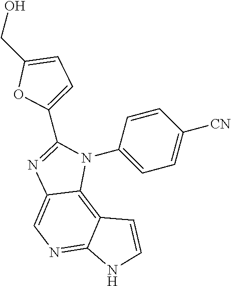 [SMILES: N#Cc1ccc(-n2c(-c3ccc(CO)o3)nc3cnc4[nH]ccc4c32)cc1] |
| 4-(2-(3-Hydroxyphenyl)imidazo[4,5- d]pyrrolo[2,3-b]pyridin-1(6H)- yl)benzonitrile | 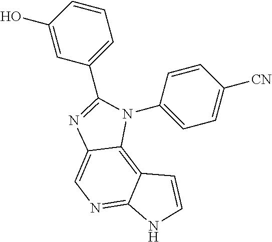 [SMILES: N#Cc1ccc(-n2c(-c3cccc(O)c3)nc3cnc4[nH]ccc4c32)cc1] |
| 4-(2-(4-(Methylsulfonyl)phenyl)-2,3- dihydroimidazo[4,5-d]pyrrolo[2,3- b]pyridin-1(6H)-yl)benzonitrile | 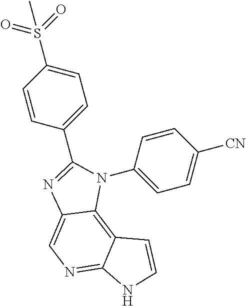 [SMILES: CS(=O)(=O)c1ccc(-c2nc3cnc4[nH]ccc4c3n2-c2ccc(C#N)cc2)cc1] |
| 3-((R)-3-(2-((R)-1- Hydroxyethyl)imidazo[4,5- d]pyrrolo[2,3-b]pyridin-1(6H)- yl)pyrrolidin-1-yl)propanenitrile | 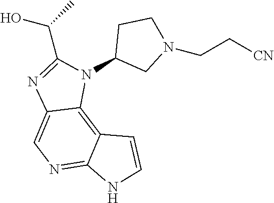 [SMILES: C[C@@H](O)c1nc2cnc3[nH]ccc3c2n1[C@H]1CCN(CCC#N)C1] |
| 3-((S)-3-(2-((R)-1- hydroxyethyl)imidazo[4,5- d]pyrrolo[2,3-b]pyridin-1(6H)- yl)pyrrolidin-1-yl)propanenitrile | 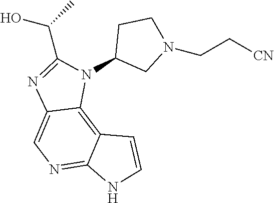 [SMILES: C[C@@H](O)c1nc2cnc3[nH]ccc3c2n1[C@H]1CCN(CCC#N)C1] |
| 3-(3-(2-(4-(Methylsulfonyl)phenyl)-2,3- dihydroimidazo[4,5-d]pyrrolo[2,3- b]pyridin-1(6H)-yl)pyrrolidin-1- yl)propanenitrile | 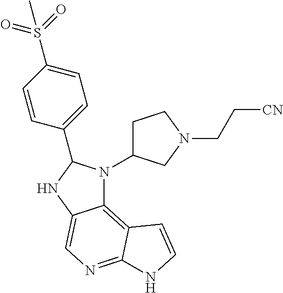 [SMILES: CS(=O)(=O)c1ccc(C2Nc3cnc4[nH]ccc4c3N2C2CCN(CCC#N)C2)cc1] |
| 1-(3-(2-((R)-1- Hydroxyethyl)imidazo[4,5- d]pyrrolo[2,3-b]pyridin-1(6H)- yl)pyrrolidin-1- carbonyl)cyclopropanecarbonitrile | 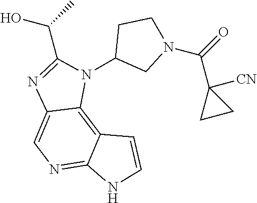 [SMILES: C[C@@H](O)c1nc2cnc3[nH]ccc3c2n1C1CCN(C(=O)C2(C#N)CC2)C1] |
| 2-(1-(Ethylsulfonyl)-3-(4-(2-(5- methylthien-2-yl)imidazo[4,5- d]pyrrolo[2,3-b]pyridin-1(6H)-yl)-1H- pyrazol-1-yl)azetidin-3-yl)acetonitrile | 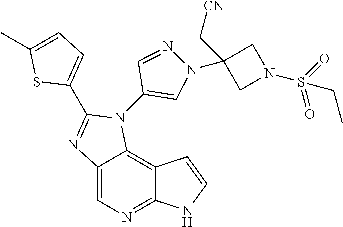 [SMILES: CCS(=O)(=O)N1CC(CC#N)(n2cc(-n3c(-c4ccc(C)s4)nc4cnc5[nH]ccc5c43)cn2)C1] |
| 2-(1-(Ethylsulfonyl)-3-(4-(2-(thiophen- 2-yl)imidazo[4,5-d]pyrrolo[2,3- b]pyridin-1(6H)-yl)-1H-pyrazol-1- yl)azetidin-3-yl)acetonitrile | 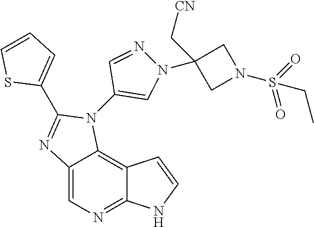 [SMILES: CCS(=O)(=O)N1CC(CC#N)(n2cc(-n3c(-c4cccs4)nc4cnc5[nH]ccc5c43)cn2)C1] |
| 3-(4-(2-(Trifluoromethyl)imidazo[4,5- d]pyrrolo[2,3-b]pyridin-1(6H)-yl)-1H- pyrazol-1-yl)propanenitrile | 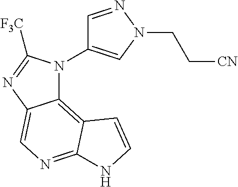 [SMILES: N#CCCn1cc(-n2c(C(F)(F)F)nc3cnc4[nH]ccc4c32)cn1] |
| 4-((S)-3-(2-((R)-1- Hydroxyethyl)imidazo[4,5- d]pyrrolo[2,3-b]pyridin-1(6H)- yl)pyrrolidin-1-yl)butanenitrile | 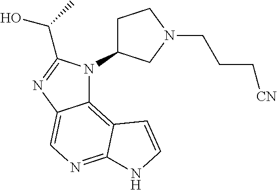 [SMILES: C[C@@H](O)c1nc2cnc3[nH]ccc3c2n1[C@H]1CCN(CCCC#N)C1] |
| 3-Cyclopentyl-3-(4-(2- (trifluoromethyl)imidazo[4,5- d]pyrrolo[2,3-b]pyridin-1(6H)-yl)-1H- pyrazol-1-yl)propanenitrile | 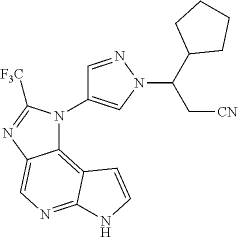 [SMILES: N#CCC(C1CCCC1)n1cc(-n2c(C(F)(F)F)nc3cnc4[nH]ccc4c32)cn1] |
| 3-(4-(2-(4-(Methylsulfonyl)phenyl)-2,3- dihydroimidazo[4,5-d]pyrrolo[2,3- b]pyridin-1(6H)-yl)-1H-pyrazol-1- yl)propanenitrile | 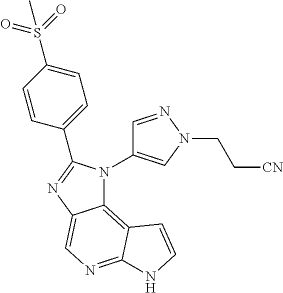 [SMILES: CS(=O)(=O)c1ccc(-c2nc3cnc4[nH]ccc4c3n2-c2cnn(CCC#N)c2)cc1] |
| 5-((S)-3-(2-((R)-1- Hydroxyethyl)imidazo[4,5- d]pyrrolo[2,3-b]pyridin-1(6H)- yl)pyrrolidin-1-yl)pentanonitrile | 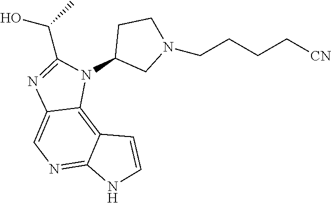 [SMILES: C[C@@H](O)c1nc2cnc3[nH]ccc3c2n1[C@H]1CCN(CCCCC#N)C1] |
| 4-((S)-7-(2-((R)-1- Hydroxyethyl)imidazo[4,5- d]pyrrolo[2,3-b]pyridin-1(6H)-yl)-5- azaspiro[2.4]hept-5-yl)butanenitrile | 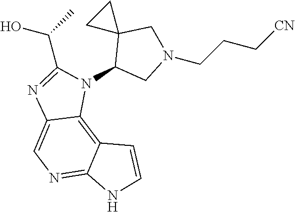 [SMILES: C[C@@H](O)c1nc2cnc3[nH]ccc3c2n1[C@@H]1CN(CCCC#N)CC12CC2] |
| (R)-1-(1-((S)-5-(4,4,4-Trifluorobutyl)- 5-azaspiro[2.4]hept-7-yl)-1,6- dihydroimidazo[4,5-d]pyrrolo[2,3- b]pyridin-2-yl)ethanol | 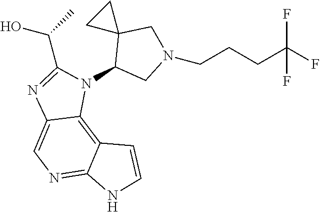 [SMILES: C[C@@H](O)c1nc2cnc3[nH]ccc3c2n1[C@@H]1CN(CCCC(F)(F)F)CC12CC2] |
| (R)-1-(1-((S)-1-(4,4,4- Trifluorobutyl)pyrrolidin-3-yl)-1,6- dihydroimidazo[4,5-d]pyrrolo[2,3- b]pyridin-2-yl)ethanol | 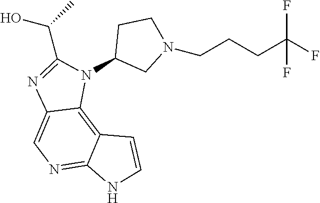 [SMILES: C[C@@H](O)c1nc2cnc3[nH]ccc3c2n1[C@H]1CCN(CCCC(F)(F)F)C1] |
| 5-((S)-7-(2-((R)-1- Hydroxyethyl)imidazo[4,5- d]pyrrolo[2,3-b]pyridin-1(6H)-yl)-5- azaspiro[2.4]hept-5-yl)pentanonitrile | 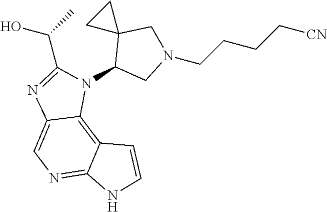 [SMILES: C[C@@H](O)c1nc2cnc3[nH]ccc3c2n1[C@@H]1CN(CCCCC#N)CC12CC2] |
| (R)-1-(1-((S)-1-(5- Fluoropentyl)pyrrolidin-3-yl)-1,6- dihydroimidazo[4,5-d]pyrrolo[2,3- b]pyridin-2-yl)ethanol | 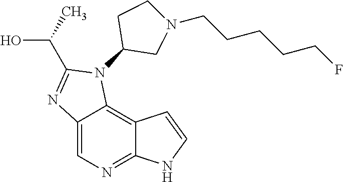 [SMILES: C[C@@H](O)c1nc2cnc3[nH]ccc3c2n1[C@H]1CCN(CCCCCF)C1] |
| 2-((S)-3-(2-((R)-1- Hydroxyethyl)imidazo[4,5- d]pyrrolo[2,3-b]pyridin-1(6H)- yl)pyrrolidin-1-yl)-N-(2,2,2- trifluoroethyl)acetamide | 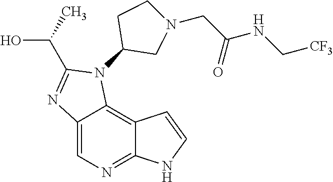 [SMILES: C[C@@H](O)c1nc2cnc3[nH]ccc3c2n1[C@H]1CCN(CC(=O)NCC(F)(F)F)C1] |
| 2-((S)-3-(2-((R)-1- Hydroxyethyl)imidazo[4,5- d]pyrrolo[2,3-b]pyridin-1(6H)- yl)pyrrolidin-1-yl)-N-(2,2,2- trifluoroethyl)propanamide | 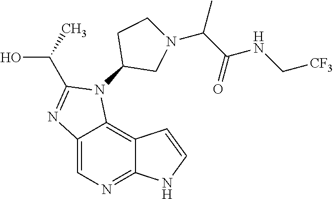 [SMILES: CC(C(=O)NCC(F)(F)F)N1CC[C@H](n2c([C@@H](C)O)nc3cnc4[nH]ccc4c32)C1] |
| 2-((S)-3-(2-((R)-1- Hydroxyethyl)imidazo[4,5- d]pyrrolo[2,3-b]pyridin-1(6H)- yl)pyrrolidin-1-yl)-N-(2,2,2- trifluoroethyl)butanamide | 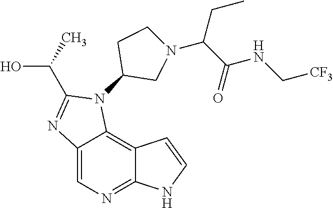 [SMILES: CCC(C(=O)NCC(F)(F)F)N1CC[C@H](n2c([C@@H](C)O)nc3cnc4[nH]ccc4c32)C1] |
| 2-(1-(ethylsulfonyl)-3-(4-(2-(5- methylfuran-2-yl)imidazo[4,5- d]pyrrolo[2,3-b]pyridin-1(6H)-yl)-1H- pyrazol-1-yl)azetidin-3-yl)acetonitrile | 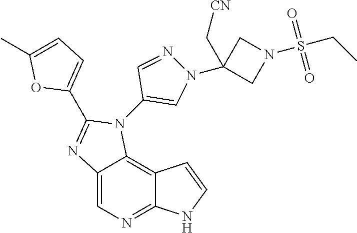 [SMILES: CCS(=O)(=O)N1CC(CC#N)(n2cc(-n3c(-c4ccc(C)o4)nc4cnc5[nH]ccc5c43)cn2)C1] |
| 2-(4-(2-(Trifluoromethyl)imidazo[4,5- d]pyrrolo[2,3-b]pyridin-1(6H)-yl)-1H- pyrazol-1-yl)acetonitrile | 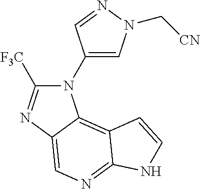 [SMILES: N#CCn1cc(-n2c(C(F)(F)F)nc3cnc4[nH]ccc4c32)cn1] |
| 3-(4-(Imidazo[4,5-d]pyrrolo[2,3- b]pyridin-1(6H)-yl)-1H-pyrazol-1- yl)propanenitrile | 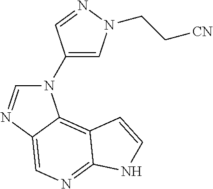 [SMILES: N#CCCn1cc(-n2cnc3cnc4[nH]ccc4c32)cn1] |
| 3-(4-(2-(2-Chlorophenyl)imidazo[4,5- d]pyrrolo[2,3-b]pyridin-1(6H)-yl)-1H- pyrazol-1-yl)propanenitrile | 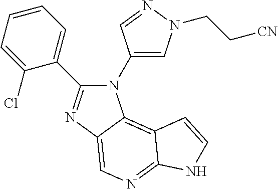 [SMILES: N#CCCn1cc(-n2c(-c3ccccc3Cl)nc3cnc4[nH]ccc4c32)cn1] |
| 2-(4-(2-(5-(Hydroxymethyl)furan-2- yl)imidazo[4,5-d]pyrrolo[2,3-b]pyridin- 1(6H)-yl)-1H-pyrazol-1-yl)acetonitrile | 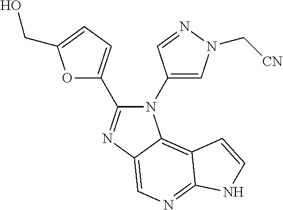 [SMILES: N#CCn1cc(-n2c(-c3ccc(CO)o3)nc3cnc4[nH]ccc4c32)cn1] |
| 3-(4-(2-(3-Hydroxyphenyl)imidazo[4,5- d]pyrrolo[2,3-b]pyridin-1(6H)-yl)-1H- pyrazol-1-yl)propanenitrile | 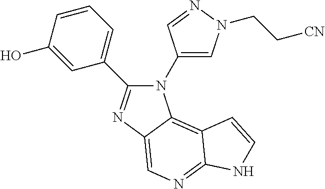 [SMILES: N#CCCn1cc(-n2c(-c3cccc(O)c3)nc3cnc4[nH]ccc4c32)cn1] |
| 3-(4-(2-(2-Fluorophenyl)imidazo[4,5- d]pyrrolo[2,3-b]pyridin-1(6H)-yl)-1H- pyrazol-1-yl)propanenitrile | 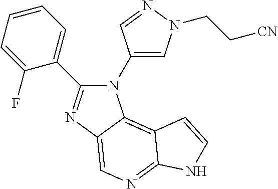 [SMILES: N#CCCn1cc(-n2c(-c3ccccc3F)nc3cnc4[nH]ccc4c32)cn1] |
| 3-(4-(2-(5-methylthiophen-2- yl)imidazo[4,5-d]pyrrolo[2,3-b]pyridin- 1(6H)-yl)-1H-pyrazol-1- yl)propanenitrile | 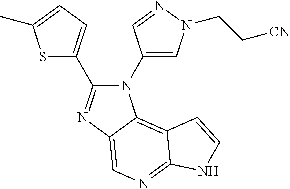 [SMILES: Cc1ccc(-c2nc3cnc4[nH]ccc4c3n2-c2cnn(CCC#N)c2)s1] |
| 4-((S)-3-(2-((R)-1- Hydroxyethyl)imidazo[4,5- d]pyrazolo[3,4-b]pyridin-1(6H)- yl)pyrrolidin-1-yl)butanenitrile | 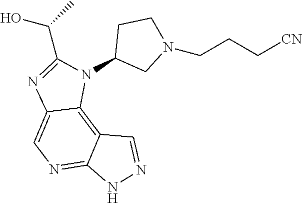 [SMILES: C[C@@H](O)c1nc2cnc3[nH]ncc3c2n1[C@H]1CCN(CCCC#N)C1] |
| (1R)-1-(1-((3R)-4-fluoro-1-(4,4,4- trifluorobutyl)pyrrolidin-3-yl)-1,6- dihydroimidazo[4,5-d]pyrrolo[2,3- b]pyridin-2-yl)ethan-1-ol | 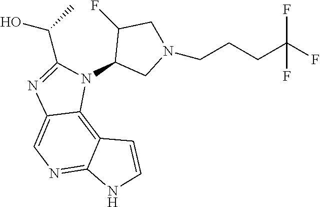 [SMILES: C[C@@H](O)c1nc2cnc3[nH]ccc3c2n1[C@@H]1CN(CCCC(F)(F)F)CC1F] |
| (R)-1-(1-((3R,4R)-4-fluoro-1-(4,4,4- trifluorobutyl)pyrrolidin-3-yl)-1,6- dihydroimidazo[4,5-d]pyrrolo[2,3- b]pyridin-2-yl)ethan-1-ol | 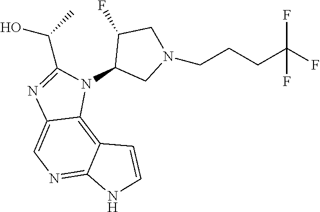 [SMILES: C[C@@H](O)c1nc2cnc3[nH]ccc3c2n1[C@@H]1CN(CCCC(F)(F)F)C[C@H]1F] |
| (R)-1-(1-((3R,4S)-4-fluoro-1-(4,4,4- trifluorobutyl)pyrrolidin-3-yl)-1,6- dihydroimidazo[4,5-d]pyrrolo[2,3- b]pyridin-2-yl)ethan-1-ol | 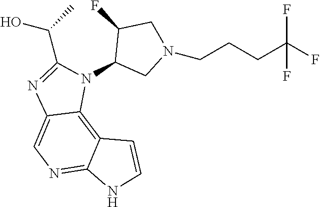 [SMILES: C[C@@H](O)c1nc2cnc3[nH]ccc3c2n1[C@@H]1CN(CCCC(F)(F)F)C[C@@H]1F] |
| (R)-1-(1-((3S,5S)-5-(hydroxymethyl)-1- (4,4,4-trifluorobutyl)pyrrolidin-3-yl)- 1,6-dihydroimidazo[4,5-d]pyrrolo[2,3- b]pyridin-2-yl)ethan-1-ol | 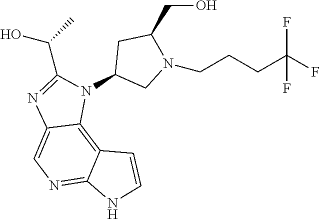 [SMILES: C[C@@H](O)c1nc2cnc3[nH]ccc3c2n1[C@H]1C[C@@H](CO)N(CCCC(F)(F)F)C1] |
| (R)-1-(1-((S)-1-(4,4,4- Trifluorobutyl)pyrrolidin-3-yl)-1,6- dihydroimidazo[4,5-d]pyrazolo[3,4- b]pyridin-2-yl)ethanol | 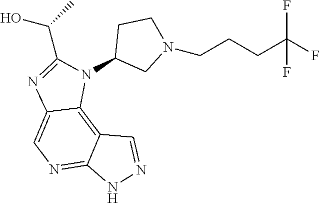 [SMILES: C[C@@H](O)c1nc2cnc3[nH]ncc3c2n1[C@H]1CCN(CCCC(F)(F)F)C1] |
| (R)-1-(1-((3S,5R)-5-(hydroxymethyl)-1- (4,4,4-trifluorobutyl)pyrrolidin-3-yl)- 1,6-dihydroimidazo[4,5-d]pyrrolo[2,3- b]pyridin-2-yl)ethan-1-ol | 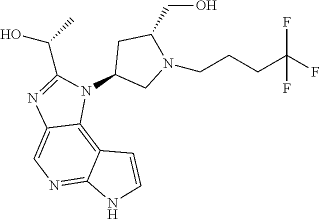 [SMILES: C[C@@H](O)c1nc2cnc3[nH]ccc3c2n1[C@H]1C[C@H](CO)N(CCCC(F)(F)F)C1] |
| (R)-1-(1-((3S,5R)-5-(hydroxymethyl)-1- (4,4,4-trifluorobutyl)pyrrolidin-3-yl)- 1,6-dihydroimidazo[4,5-d]pyrrolo[2,3- b]pyridin-2-yl)ethan-1-ol | 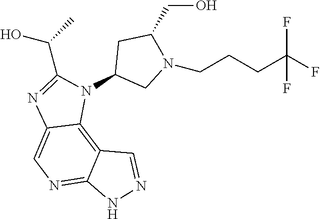 [SMILES: C[C@@H](O)c1nc2cnc3[nH]ncc3c2n1[C@H]1C[C@H](CO)N(CCCC(F)(F)F)C1] |
| 4-((4R)-3-Fluoro-4-(2-((R)-1- hydroxyethyl)imidazo[4,5- d]pyrazolo[3,4-b]pyridin-1(6H)-yl) pyrrolidin-1-yl)butanenitrile | 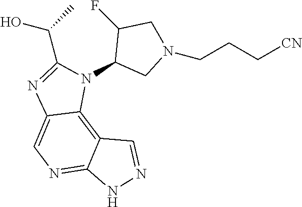 [SMILES: C[C@@H](O)c1nc2cnc3[nH]ncc3c2n1[C@@H]1CN(CCCC#N)CC1F] |
| 4-((28,4S)-4-(2-((R)-1- Hydroxyethyl)imidazo[4,5- d]pyrazolo[3,4-b]pyridin-1(6H)-yl)-2- (hydroxymethyl)pyrrolidin-1- yl)butanenitrile | 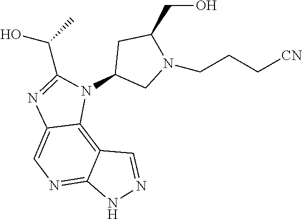 [SMILES: C[C@@H](O)c1nc2cnc3[nH]ncc3c2n1[C@H]1C[C@@H](CO)N(CCCC#N)C1] |
| trans-4-(2-(3,4- Difluorophenyl)imidazo[4,5- d]pyrrolo[2,3-b]pyridin-1(6H)- yl)cyclohexanecarbonitrile | 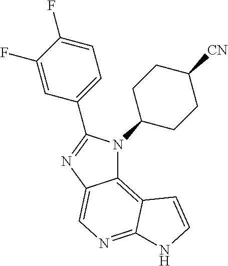 [SMILES: N#C[C@H]1CC[C@@H](n2c(-c3ccc(F)c(F)c3)nc3cnc4[nH]ccc4c32)CC1] |
| (R)-1-(1-((S)-1-(3,3,3- Trifluoropropyl)pyrrolidin-3-yl)-1,6- dihydroimidazo[4,5-d]pyrrolo[2,3- b]pyridin-2-yl)ethanol | 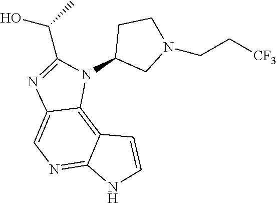 [SMILES: C[C@@H](O)c1nc2cnc3[nH]ccc3c2n1[C@H]1CCN(CCC(F)(F)F)C1] |
| (R)-1-(1-((S)-1-(3,3,4,4,4- Pentafluorobutyl)pyrrolidin-3-yl)-1,6- dihydroimidazo[4,5-d]pyrrolo[2,3- b]pyridin-2-yl)ethanol | 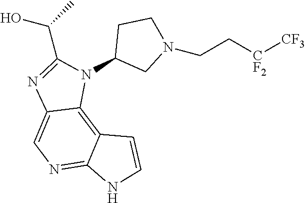 [SMILES: C[C@@H](O)c1nc2cnc3[nH]ccc3c2n1[C@H]1CCN(CCC(F)(F)C(F)(F)F)C1] |
| (S)-(5-(1-(1-(4,4,4- trifluorobutyl)pyrrolidin-3-yl)-1,6- dihydroimidazo[4,5-d]pyrrolo[2,3- b]pyridin-2-yl)furan-2-yl)methanol | 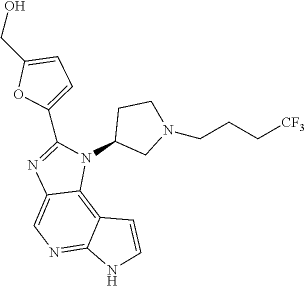 [SMILES: OCc1ccc(-c2nc3cnc4[nH]ccc4c3n2[C@H]2CCN(CCCC(F)(F)F)C2)o1] |
| (S)-3-(1-(1-(4,4,4- trifluorobutyl)pyrrolidin-3-yl)-1,6- dihydroimidazo[4,5-d]pyrrolo[2,3- b]pyridin-2-yl)phenol | 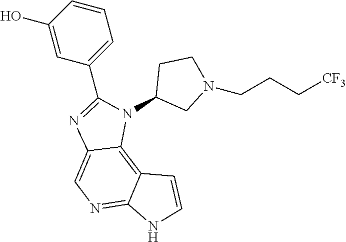 [SMILES: Oc1cccc(-c2nc3cnc4[nH]ccc4c3n2[C@H]2CCN(CCCC(F)(F)F)C2)c1] |
| (R)-1-(1-((S)-1-(2,2,3,3,4,4,4- heptafluorobutyl)pyrrolidin-3-yl)-1,6- dihydroimidazo[4,5-d]pyrrolo[2,3- b]pyridin-2-yl)ethanol | 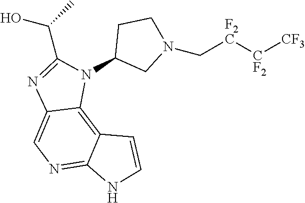 [SMILES: C[C@@H](O)c1nc2cnc3[nH]ccc3c2n1[C@H]1CCN(CC(F)(F)C(F)(F)C(F)(F)F)C1] |
| (R)-1-(1-((S)-1-(Perfluoro- butyl)pyrrolidin-3-yl)-1,6- dihydroimidazo[4,5-d]pyrrolo[2,3- b]pyridin-2-yl)ethanol | 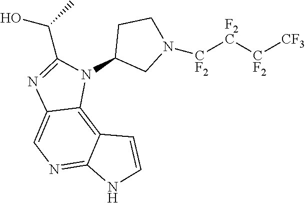 [SMILES: C[C@@H](O)c1nc2cnc3[nH]ccc3c2n1[C@H]1CCN(C(F)(F)C(F)(F)C(F)(F)C(F)(F)F)C1] |
| (R)-1-(1-((S)-1-((3,3,3- Trifluoropropyl)sulfonyl)pyrrolidin-3- yl)-1,6-dihydroimidazo[4,5- d]pyrrolo[2,3-b]pyridin-2-yl)ethanol | 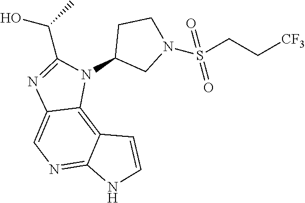 [SMILES: C[C@@H](O)c1nc2cnc3[nH]ccc3c2n1[C@H]1CCN(S(=O)(=O)CCC(F)(F)F)C1] |
| 4-(4-(Imidazo[4,5-d]pyrrolo[2,3- b]pyridin-1(6H)-yl)-1H-pyrazol-1- yl)butanenitrile | 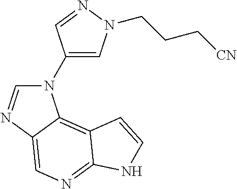 [SMILES: N#CCCCn1cc(-n2cnc3cnc4[nH]ccc4c32)cn1] |
| 4-(4-(2-(5-methylfuran-2- yl)imidazo[4,5-d]pyrrolo[2,3-b]pyridin- 1(6H)-yl)-1H-pyrazol-1-yl)butanenitrile | 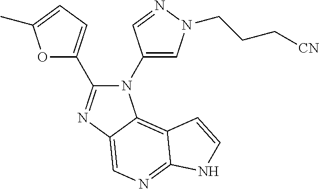 [SMILES: Cc1ccc(-c2nc3cnc4[nH]ccc4c3n2-c2cnn(CCCC#N)c2)o1] |
| 4-(4-(2-(5-Methylthiophen-2- yl)imidazo[4,5-d]pyrrolo[2,3-b]pyridin- 1(6H)-yl)-1H-pyrazol-1-yl butanenitrile | 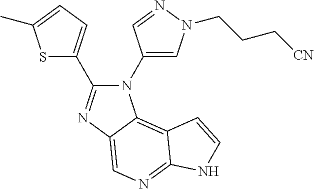 [SMILES: Cc1ccc(-c2nc3cnc4[nH]ccc4c3n2-c2cnn(CCCC#N)c2)s1] |
| 3-(4-(2-(Thiophen-2-yl)imidazo[4,5- d]pyrrolo[2,3-b]pyridin-1(6H)-yl)-1H- pyrazol-1-yl)propanenitrile | 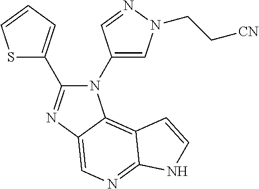 [SMILES: N#CCCn1cc(-n2c(-c3cccs3)nc3cnc4[nH]ccc4c32)cn1] |
| 4-(4-(2-Cyclobutylimidazo[4,5- d]pyrrolo[2,3-b]pyridin-1(6H)-yl)-1H- pyrazol-1-yl)butanenitrile | 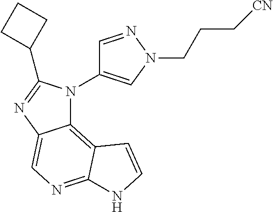 [SMILES: N#CCCCn1cc(-n2c(C3CCC3)nc3cnc4[nH]ccc4c32)cn1] |
| 4-(4-(2-Cyclopropylimidazo[4,5- d]pyrrolo[2,3-b]pyridin-1(6H)-yl)-1H- pyrazol-1-yl)butanenitrile | 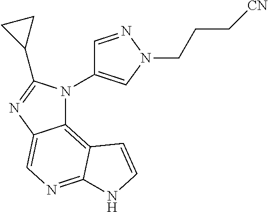 [SMILES: N#CCCCn1cc(-n2c(C3CC3)nc3cnc4[nH]ccc4c32)cn1] |

10. A method of preparing a compound as shown in Formula I, which includes the following step: reacting a compound as shown in Formula if with a base in an organic solvent to obtain said compound as shown in Formula I; 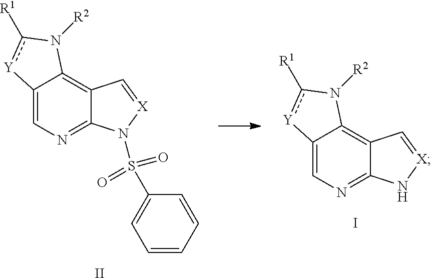
wherein X, Y, [symbol] , R¹ and R² are defined as described in claim 1.

11. A compound as shown in Formula 1I: 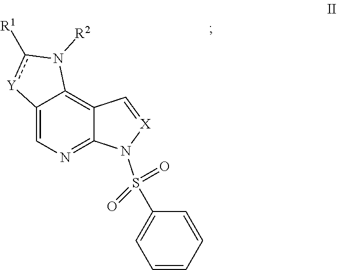
wherein X, Y, [symbol] , R¹ and R² are defined as described in claim 1.

12. A pharmaceutical composition comprising
(i) the compound as claimed in claim 1, or a tautomer, stereoisomer, racemate or isotopic derivative thereof, or a pharmaceutically acceptable salt of any of the foregoing, or a crystalline or solvated form of any of the foregoing; and
(ii) a pharmaceutically acceptable vehicle.

13. A method for inhibiting Janus kinase in vivo, or in vitro, comprising: contacting the compound as claimed in at claim 1, or a tautomer, stereoisomer, racemate or isotopic derivative thereof, or a pharmaceutically acceptable salt of any of the foregoing, or a crystalline or solvated form of any of the foregoing, with Janus kinase.

14. A method for treating diseases associated with Janus kinase in a subject in need thereof, comprising, administering a therapeutically effective amount of the compound as claimed in claim 1, or a tautomer, stereoisomer, racemate or isotopic derivative thereof, or a pharmaceutically acceptable salt of any of the foregoing, or a crystalline or solvated form of any of the foregoing to the subject.

15. The method as claimed in claim 14, wherein the mentioned diseases associated with Janus kinase is autoimmune diseases or cancer.

16. A method for inhibiting Janus kinase in vivo or in vitro, comprising: contacting the pharmaceutical composition as claimed in claim 12 with Janus kinase.

17. A method for treating diseases associated with Janus kinase in a subject in need thereof, comprising: administering a therapeutically effective amount of the pharmaceutical composition as claimed in claim 12.

18. The method as claimed in claim 17, wherein the mentioned diseases associated with Janus kinase is autoimmune diseases or cancer.
